(19) United States
(12) Patent Application Publication
(10) Pub. No.: US 2024/0228489 A1
(43) Pub. Date: Jul. 11, 2024

(54) POSITIVE ALLOSTERIC MODULATORS OF THE MUSCARINIC ACETYLCHOLINE RECEPTOR M1

(71) Applicant: Vanderbilt University, Nashville, TN (US)

(72) Inventors: Craig W. Lindsley, Brentwood, TN (US); Darren W. Engers, Brentwood, TN (US); Julie L. Engers, Brentwood, TN (US); Changho Han, Nashville, TN (US); Alison R. Gregro, Mount Juliet, TN (US); Madeline F. Long, Nashville, TN (US); Jinming Li, Nashville, TN (US); Joseph D. Bungard, Nashville, TN (US); Cayden J. Dodd, Nashville, TN (US)

(21) Appl. No.: 18/554,867

(22) PCT Filed: Apr. 14, 2022
(86) PCT No.: PCT/US2022/024854

§ 371 Date: Oct. 11, 2023

Related U.S. Application Data
(60) Provisional application No. 63/174,931, filed on Apr. 14, 2021.

(51) Int. Cl. C07D 471/04; A61K 31/437; A61K 31/444; C07D 519/00

(52) U.S. Cl. CPC C07D 471/04; A61K 31/437; A61K 31/444; C07D 519/00

(57) ABSTRACT

Described are deuterium-labeled 6,7-dihydro-5H-pyrrolo[3,4-b]pyridin-5-one and isoindolin-1-one positive allosteric modulators of muscarinic acetylcholine receptor M₁(mAChR M₁), pharmaceutical compositions including the compounds, and methods of using the compounds and compositions for treating neurological disorders, psychiatric disorders, or a combination thereof.

### RELATED APPLICATIONS

This application claims priority to U.S. Provisional Application No. 63/174,931, filed Apr. 14, 2021, which is hereby incorporated by reference in its entirety.

### TECHNICAL FIELD

The present disclosure relates to compounds, compositions, and methods for treating muscarinic acetylcholine receptor M₁ related diseases and/or disorders, such as neurological and psychiatric disorders.

### BACKGROUND

Positive allosteric modulators are compounds that bind to a site distinct from that of the orthosteric agonist binding site of a target protein. These modulators enhance the affinity or efficacy of an orthosteric agonist. For example, a selective muscarinic M₁ positive allosteric modulator would result in an increased affinity at the orthosteric binding site for acetylcholine (ACh), the endogenous agonist for the muscarinic M₁ receptor, or an increase in the efficacy induced by ACh. In some systems, the compound may also have an intrinsic activity to activate the receptor in the absence of orthosteric ligand. Positive allosteric modulation (potentiation), therefore, can be an attractive mechanism for enhancing appropriate physiological receptor activation.

Cholinergic neurotransmission involves the activation of nicotinic acetylcholine receptors (nAChRs) or the muscarinic acetylcholine receptors (mAChRs) by the binding of the endogenous orthosteric agonist ACh. Acetylcholinesterase (AChE) inhibitors, which inhibit the hydrolysis of ACh, have been approved in the United States for use in the palliative, but not disease-modifying, treatment of the cognitive deficits in Alzheimer's disease (AD) patients.

mAChRs are members of the family A GPCRs, and include five subtypes, designated M₁, M₂, M₃, M₄, and M₅. M₁, M₃ and M₅ mainly couple to Gq and activate phospholipase C, whereas M₂ and M₄ mainly couple to G_{i/o} and associated effector systems. These five distinct mAChR subtypes have been identified in the mammalian central nervous system where they are prevalent and differentially expressed. M₁-M₅ mAChRs have varying roles in cognitive, sensory, motor and autonomic functions. Activation of various muscarinic receptors, particularly the M₁ subtype, has been proposed as a mechanism to enhance cognition in disorders such as AD and schizophrenia (as well as negative symptoms). Thus, selective positive allosteric modulators of mAChR subtypes that regulate processes involved in cognitive function could prove superior to AChE inhibitors for treatment of AD and related disorders as these compounds may exhibit improved selectivity for specific mAChRs.

Efforts to create selective M₁ agonists have been largely unsuccessful, in part due to the high conservation of the orthosteric ACh binding site. As a result, mAChR agonists in clinical studies induce the same adverse effects of AChE inhibitors by activation of peripheral mAChRs. To fully understand the physiological roles of individual mAChR subtypes and to further explore the therapeutic utility of mACh receptors in AD, schizophrenia and other disorders, there exists a need to develop compounds that are highly selective modulators of M₁ and other individual mAChR subtypes. Accordingly, allosteric modulation may be an advantageous pathway because allosteric sites on mAChRs are less highly conserved.

Despite advances in muscarinic receptor (mAChR) research, there remains a scarcity of compounds that are potent, efficacious and selective positive allosteric modulators of the M₁ mAChR that are also effective in the treatment of neurological and psychiatric disorders associated with cholinergic activity, or other neurologic diseases in which the muscarinic M₁ receptor may be involved.

### SUMMARY

In one aspect, disclosed are compounds of formula (I), or pharmaceutically acceptable salts thereof,

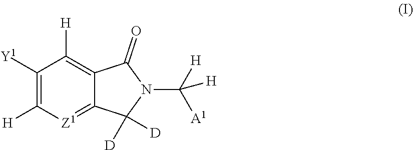

wherein
A¹ is

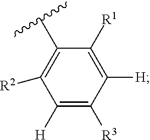

R¹ and R² are independently selected from the group consisting of H, D, C₁₋₄alkyl, CD₃, C₁₋₂fluoroalkyl, —OR^{2a}, OCD₃, halogen, C₃₋₅cycloalkyl, and —N(R^{2a})₂;
R^{2a}, at each occurrence, is independently selected from the group consisting of H, C₁₋₄alkyl, and C₁₋₄fluoroalkyl, wherein alternatively, two R^{2a}, together with a nitrogen to which the R^{2a} attach, form an azetidinyl, pyrrolidinyl, or piperidinyl;
R³ is a ring system selected from an aromatic or partially unsaturated 9-membered fused bicyclic heterocyclic ring system containing 1-3 nitrogen atoms, the heterocyclic ring system being unsubstituted or substituted with 1-4 substituents independently selected from the group consisting of halogen, C₁₋₆alkyl, C₁₋₆deuterioalkyl, C₁₋₆haloalkyl, oxo, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl;
R^{3a} is C₁₋₆alkyl or C₁₋₆deuterioalkyl, each independently of each other unsubstituted or substituted with a substituent selected from the group consisting of —OR^{3b}, —N(R^{3b})₂, —SR^{3b}, —SO₂R^{3c}, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl, wherein each heterocyclyl is a 4- to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl are unsubstituted or substituted with 1-4 substituents independently selected from the group consisting of halogen, cyano, C₁₋₄alkyl, C₁₋₄deuterioalkyl, C₁₋₄haloalkyl, C₃₋₆cycloalkyl, oxo, —OR^{3b}, —N(R^{3b})₂, —NR^{3b}C(O)R^{3b}, —C(O)N(R^{3b})₂, —C(O)OR^{3b}, —SO₂R^{3c}, and C(O)R^{3b};
R^{3b}, at each occurrence, is independently selected from the group consisting of hydrogen, C₁₋₄alkyl, C₁₋₄deuterioalkyl, C₁₋₄haloalkyl, and C₃₋₄cycloalkyl;
R^{3c}, at each occurrence, is independently selected from the group consisting of C₁₋₄alkyl, C₁₋₄deuterioalkyl, C₁₋₄haloalkyl, and C₃₋₄cycloalkyl;
Y¹ is selected from the group consisting of H, CH₃, CD₃, CF₃, OH, OCH₃, OCD₃, and halogen; and
Z¹ is N or C—H.

In another aspect, the invention provides compounds of formula (II), or pharmaceutically acceptable salts thereof,

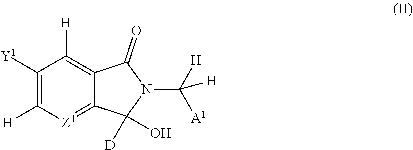

wherein
A¹ is

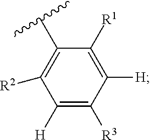

R¹ and R² are independently selected from the group consisting of H, D, C₁₋₄alkyl, CD₃, C₁₋₂fluoroalkyl, —OR^{2a}, OCD₃, halogen, C₃₋₅cycloalkyl, and —N(R^{2a})₂,
R^{2a}, at each occurrence, is independently selected from the group consisting of H, C₁₋₄alkyl, and C₁₋₄fluoroalkyl, wherein alternatively, two R^{2a}, together with a nitrogen to which the R^{2a} attach, form an azetidinyl, pyrrolidinyl, or piperidinyl;
R³ is a ring system selected from an aromatic or partially unsaturated 9-membered fused bicyclic heterocyclic ring system containing 1-3 nitrogen atoms, the heterocyclic ring system being unsubstituted or substituted with 1-4 substituents independently selected from the group consisting of halogen, C₁₋₆alkyl, C₁₋₆deuterioalkyl, C₁₋₆haloalkyl, oxo, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl;
R^{3a} is C₁₋₆alkyl or C₁₋₆deuterioalkyl, each independently of each other unsubstituted or substituted with a substituent selected from the group consisting of —OR^{3b}, —N(R^{3b})₂—SR^{3b}, —SO₂R^{3c}, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl, wherein each heterocyclyl is a 4 to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl are unsubstituted or substituted with 1-4 substituents independently selected from the group consisting of halogen, cyano, C₁₋₄alkyl, C₁₋₄deuterioalkyl, C₁₋₄haloalkyl, C₃₋₆cycloalkyl, oxo, —OR^{3b}, —N(R^{3b})₂, —NR^{3b}C(O)R^{3b}, —C(O)N(R^{3b})₂, —C(O)OR^{3b}, —SO₂R^{3c}, and C(O)R^{3b};
R^{3b}, at each occurrence, is independently selected from the group consisting of hydrogen, C₁₋₄alkyl, C₁₋₄deuterioalkyl, C₁₋₄haloalkyl, and C₃₋₄cycloalkyl;
R^{3c}, at each occurrence, is independently selected from the group consisting of C₁₋₄alkyl, C₁₋₄deuterioalkyl, C₁₋₄haloalkyl, and C₃₋₄cycloalkyl;
Y¹ is selected from the group consisting of H, CH₃, CD₃, CF₃, OH, OCH₃, OCD₃, and halogen; and
Z¹ is N or C—H.

In another aspect, the invention provides a pharmaceutical composition comprising a compound of formula (I) or (II), or a pharmaceutically acceptable salt thereof, and a pharmaceutically acceptable carrier.

In another aspect, the invention provides a method for the treatment of a disorder associated with muscarinic acetylcholine receptor activity in a mammal, comprising administering to the mammal an effective amount of a compound of formula (I) or (II), or a pharmaceutically acceptable salt or composition thereof.

In another aspect, the invention provides a compound of formula (I) or (II), or a pharmaceutically acceptable salt or composition thereof, for use in the treatment of a disorder associated with muscarinic acetylcholine receptor activity.

In another aspect, the invention provides the use of a compound of formula (I) or (II), or a pharmaceutically acceptable salt or composition thereof, in the manufacture of a medicament for the treatment of a disorder associated with muscarinic acetylcholine receptor activity.

In another aspect, the invention provides a kit comprising a compound of formula (I) or (II), or a pharmaceutically acceptable salt or composition thereof, and instructions for use.

In another aspect, the invention provides a compound of formula (I) or (II), or a pharmaceutically acceptable salt or composition thereof, with minimal to substantially no M₁ agonist activity compared to acetylcholine. The relative absence of M₁ agonist activity is expected to avoid or reduce cholinergic adverse effect liability at therapeutic concentrations and/or doses.

### DETAILED DESCRIPTION

Disclosed herein are positive allosteric modulators of the M₁ muscarinic acetylcholine receptor M₁ (mAChR M₁). The modulators can have the structure of formula (I) or (II). Compounds of formula (I) exhibit high affinity for mAChR M₁, and can also exhibit selectivity over other muscarinic acetylcholine receptors. Compounds of formula (I) or (II) can be used to treat or prevent diseases and disorders associated with mAChR M₁ by modulating mAChR M₁ activity. mAChR M₁ has been implicated in a number of different diseases and disorders including, but not limited to, neurological and psychiatric disorders.

Since the orthosteric binding sites of the mAChR isoforms are highly conserved, selective modulators of the mAChRs that bind at the orthosteric site remain elusive. One strategy to selectively bind and modulate the mAChRs includes identifying allosteric sites which may be amenable to modulation by a small molecule. In particular, positive allosteric modulation of mAChR M₁ can result in potentiation of the mAChR M₁ receptor and provide therapeutic benefits for disorders associated with mAChR M₁ dysfunction.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5′ Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “alkenylene,” as used herein, refers to a divalent group derived from a straight or branched chain hydrocarbon having at least one carbon-carbon double bond.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), a bridged cycloalkyl in which two non-adjacent atoms of a ring are linked by an alkylene bridge of 1, 2, 3, or 4 carbon atoms (e.g., bicyclo[2.2.1]heptanyl), or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkene,” as used herein, means a non-aromatic monocyclic or multicyclic all-carbon ring system containing at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl.

The term “deuterioalkyl,” as used herein, means an alkyl group, as defined herein, in which one or more hydrogen atoms in the alkyl are the isotope deuterium, i.e., ²H. Representative examples of deuterioalkyl include CD₃, CH₂CD₃, and CD₂CD₃.

The term “fluoroalkyl,” as used herein, means an alkyl group, as defined herein, in which one, two, three, four, five, six, seven or eight hydrogen atoms are replaced by a fluoro group. Representative examples of fluoroalkyl include CH₂F, CHF₂, CF₃, and CH₂CF₃.

The term “fluorodeuterioalkyl,” as used herein, means a fluoroalkyl group, as defined herein, in which one or more hydrogen atoms in the fluoroalkyl are the isotope deuterium, i.e., ²H. Representative examples of fluorodeuterioalkyl include CD₂F, CDF₂, and CD₂CF₃.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “halodeuterioalkyl,” as used herein, means a haloalkyl, as defined herein, in which one or more hydrogen atoms in the haloalkyl are the isotope deuterium, i.e., ²H. Representative examples of halodeuterioalkyl include CD₂F, CDF₂, CD₂CF₃, CD₂Cl, CDCl₂, and CD₂CCl₃.

The term “heteroaryl,” as used herein, refers to an aromatic monocyclic heteroatom-containing ring (monocyclic heteroaryl) or a bicyclic ring system containing at least one monocyclic heteroaryl (bicyclic heteroaryl). The term “heteroaryl” is used herein to refer to a heteroarene when present as a substituent, the term “heteroarene” being used in cases of ring fusion. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl is an 8- to 12-membered ring system and includes a fused bicyclic heteroaromatic ring system (i.e., “fully aromatic” 10π electron system) such as a monocyclic heteroaryl ring fused to a 6-membered arene (e.g., quinolin-4-yl, indol-1-yl), a monocyclic heteroaryl ring fused to a monocyclic 5- to 6-membered heteroarene (e.g., naphthyridinyl), and a phenyl fused to a monocyclic 5- to 6-membered heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, and 3-oxaspiro[5.5]undecanyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. As used herein, “Cm to Cn,” “Cm-Cn” or “Cm-n” in which “m” and “n” are integers refers to the number of carbon atoms in the relevant group. That is, the group can contain from “m” to “n” inclusive, carbon atoms. Thus, for example, a “C₁ to C₆ alkyl” group refers to all alkyl groups having from 1 to 6 carbons, that is, CH₃—, CH₃CH₂—, CH₃CH₂CH₂—, (CH₃)₂CH—, CH₃CH₂CH₂CH₂—, CH₃CH₂CH(CH₃)—, CH₃CH(CH)₃CH₂—, CH₃CH(CH)₃CH₂— and (CH₃)₃C—. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched). If no “m” and “n” are designated with regard to a group, the broadest range described in these definitions is to be assumed.

A dashed bond, [symbol] , represents an optional unsaturation between the atoms forming the bond. This bond may be unsaturated (e.g. C═C, C═N, C═O) or saturated (e.g. C—C, C—N, C—O). When a dashed bond is present in a ring system it may form part of an aromatic ring system.

The term “substituted” refers to a group that may be further substituted with one or more non-hydrogen substituent groups. Substituent groups may include, for example, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

As used herein, an “optional substituent” is a substituent that may or may not be present on another molecular group, such as a ring (e.g., phenyl) or chain (e.g., alkyl). A group that is “optionally substituted” with a substituent means the group is either unsubstituted or substituted with the substituent.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Pharmaceutically acceptable solvates and hydrates are complexes of a compound with one or more solvent or water molecules, or 1 to about 100, or 1 to about 10, or one to about 2, 3, or 4, solvent or water molecules.

As used herein, a “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392).

The terms, “polymorphs” and “polymorphic forms” refer to crystalline forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation. Techniques for characterizing polymorphs include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds

Throughout the embodiments and description of the compounds of the invention, all instances of haloalkyl may be fluoroalkyl (e.g., any C₁₋₄haloalkyl may be C₁₋₄fluoroalkyl).

Unsubstituted or substituted rings such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In the following, numbered embodiments of the invention are disclosed (e.g., E1, E1.1, E1.2, E1.3, E1.4, E2, etc.). In the numbered embodiments, the reference to a range of preceding embodiments in multiple dependent format (e.g., “the compound of any of E1-E1.2”) is a reference, in the alternative, to each embodiment sequentially listed herein in the recited range. For example, the range “any of E1-E1.2” means “any of E1, E1.1, or E1.2.” Likewise, the range of “any of E1-E2” means “any of E1, E1.1, E1.2, E1.3, E1.4, or E2.”

E1. A compound of formula (I), or a pharmaceutically acceptable salt thereof,

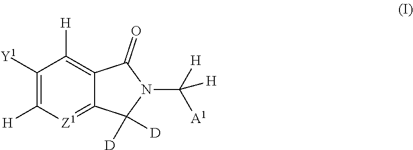

wherein
A¹ is

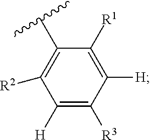

R¹ and R² are independently selected from the group consisting of H, D, C₁₋₄alkyl, CD₃, C₁₋₂fluoroalkyl, —OR^{2a}, OCD₃, halogen, C₃₋₅cycloalkyl, and —N(R^{2a})₂;
R^{2a}, at each occurrence, is independently selected from the group consisting of H, C₁₋₄alkyl, and C₁₋₄fluoroalkyl, wherein alternatively, two R^{2a}, together with a nitrogen to which the R^{2a} attach, form an azetidinyl, pyrrolidinyl, or piperidinyl;
R³ is a ring system selected from an aromatic or partially unsaturated 9-membered fused bicyclic heterocyclic ring system containing 1-3 nitrogen atoms, the heterocyclic ring system being unsubstituted or substituted with 1-4 substituents independently selected from the group consisting of halogen, C₁₋₆alkyl, C₁₋₆deuterioalkyl, C₁₋₆haloalkyl, oxo, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl;
R^{3a} is C₁₋₆alkyl or C₁₋₆deuterioalkyl, each independently of each other unsubstituted or substituted with a substituent selected from the group consisting of —OR^{3b}, —N(R^{3b})₂—SR^{3b}, —SO₂R^{3c}, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl, wherein each heterocyclyl is a 4 to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl are unsubstituted or substituted with 1-4 substituents independently selected from the group consisting of halogen, cyano, C₁₋₄alkyl, C₁₋₄deuterioalkyl, C₁₋₄haloalkyl, C₃₋₆cycloalkyl, oxo, —OR^{3b}, —N(R^{3b})₂, —NR^{3b}C(O)R^{3b}, —C(O)N(R^{3b})₂, —C(O)OR^{3b}, —SO₂R^{3c}, and C(O)R^{3b};
R^{3b}, at each occurrence, is independently selected from the group consisting of hydrogen, C₁₋₄alkyl, C₁₋₄deuterioalkyl, C₁₋₄haloalkyl, and C₃₋₄cycloalkyl;
R^{3c}, at each occurrence, is independently selected from the group consisting of C₁₋₄alkyl, C₁₋₄deuterioalkyl, C₁₋₄haloalkyl, and C₃₋₄cycloalkyl;
Y¹ is selected from the group consisting of H, CH₃, CD₃, CF₃, OH, OCH₃, OCD₃, and halogen; and
Z¹ is N or C—H.

E1.1. A compound of formula (II), or a pharmaceutically acceptable salt thereof,

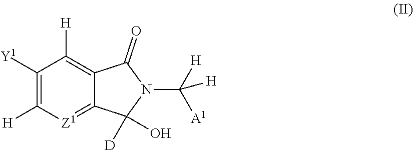

wherein
A¹ is

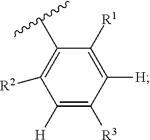

R¹ and R² are independently selected from the group consisting of H, D, C₁₋₄alkyl, CD₃, C₁₋₂fluoroalkyl, —OR^{2a}, OCD₃, halogen, C₃₋₅cycloalkyl, and —N(R^{2a})₂. R^{2a}, at each occurrence, is independently selected from the group consisting of H, C₁₋₄alkyl, and C₁₋₄fluoroalkyl, wherein alternatively, two R^{2a}, together with a nitrogen to which the R^{2a} attach, form an azetidinyl, pyrrolidinyl, or piperidinyl;
R³ is a ring system selected from an aromatic or partially unsaturated 9-membered fused bicyclic heterocyclic ring system containing 1-3 nitrogen atoms, the heterocyclic ring system being unsubstituted or substituted with 1-4 substituents independently selected from the group consisting of halogen, C₁₋₆alkyl, C₁₋₆deuterioalkyl, C₁₋₆haloalkyl, oxo, —OH, —OR³a, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl;
R^{3a} is C₁₋₆alkyl or C₁₋₆deuterioalkyl, each independently of each other unsubstituted or substituted with a substituent selected from the group consisting of —OR^{3b}, —N(R^{3b})₂, —SR^{3b}, —SO₂R^{3c}, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl, wherein each heterocyclyl is a 4 to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl are unsubstituted or substituted with 1-4 substituents independently selected from the group consisting of halogen, cyano, C₁₋₄alkyl, C₁₋₄deuterioalkyl, C₁₋₄haloalkyl, C₃₋₆cycloalkyl, oxo, —OR^{3b}, —N(R^{3b})₂, —NR^{3b}C(O)R^{3b}, —C(O)N(R^{3b})₂, —C(O)OR^{3b}, —SO₂R³, and C(O)R^{3b};
R^{3b}, at each occurrence, is independently selected from the group consisting of hydrogen, C₁₋₄alkyl, C₁₋₄deuterioalkyl, C₁₋₄haloalkyl, and C₃₋₄cycloalkyl;
R^{3c}, at each occurrence, is independently selected from the group consisting of C₁₋₄alkyl, C₁₋₄deuterioalkyl, C₁₋₄haloalkyl, and C₃₋₄cycloalkyl;
Y¹ is selected from the group consisting of H, CH₃, CD₃, CF₃, OH, OCH₃, OCD₃, and halogen; and Z¹ is N or C—H.

E1.2. The compound of E1 or E1.1, or a pharmaceutically acceptable salt thereof, wherein:
R¹ is selected from the group consisting of H, D, CH₃, CD₃, CF₃, OCH₃, OCD₃, and halogen;
R² is selected from the group consisting of H, D, CH₃, CD₃, CF₃, OCH₃, OCD₃, and halogen;
R³ is a ring system selected from a 9-membered fused bicyclic heteroaryl containing 1-3 nitrogen atoms, the heteroaryl being unsubstituted or substituted with 1-4 substituents independently selected from the group consisting of halogen, C₁₋₆alkyl, C₁₋₆deuterioalkyl, C₁₋₆haloalkyl, —OR³a, —OC₁₋₆haloalkyl, —OC₁₋₆halodeuterioalkyl, C₃₋₅cycloalkyl, —C₁₋₃alkylene-C₃₋₅cycloalkyl, and oxetan-3-yl; and
R^{3a} is C₁₋₆alkyl or C₁₋₆deuterioalkyl, each independently of each other unsubstituted or substituted with a substituent selected from the group consisting of —OR^{3b}, —N(R^{3b})₂, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, -heterocyclyl, —C₁₋₄alkylene-heterocyclyl, wherein each heterocyclyl is a 4 to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, -heterocyclyl, —C₁₋₄alkylene-heterocyclyl are unsubstituted or substituted with 1-4 substituents independently selected from the group consisting of halogen, C₁₋₄alkyl, C₁₋₄deuterioalkyl, C₁₋₄haloalkyl, C₃₋₄cycloalkyl, oxo, —OR^{3b}, —N(R^{3b})₂, —NR^{3b}C(O)R^{3b}, —C(O)N(R^{3b})₂, and C(O)R^{3b}.

E1.3. The compound of any of E1-E1.2, or a pharmaceutically acceptable salt thereof, wherein R³ is unsubstituted or substituted with 1-4 substituents independently selected from the group consisting of halogen, C₁₋₄alkyl, C₁₋₄deuterioalkyl, C₁₋₄haloalkyl, —OC₁₋₄alkyl, —OC₁₋₄deuterioalkyl, —OC₁₋₄haloalkyl, C₃₋₄cycloalkyl, —C₁₋₃alkylene-C₃₋₄cycloalkyl, and oxetan-3-yl.

E1.4. The compound of E1 or E1.1, or a pharmaceutically acceptable salt thereof, wherein
R¹ is selected from the group consisting of H, D, C₁₋₄alkyl, CD₃, C₁₋₂fluoroalkyl, —OR^{2a}, OCD₃, fluoro, chloro, C₃₋₅cycloalkyl, and —N(R^{2a})₂; and
R² is selected from the group consisting of H, D, fluoro, and chloro.

E2. The compound of any of E1-E1.4, or a pharmaceutically acceptable salt thereof, wherein A¹ is selected from the group consisting of

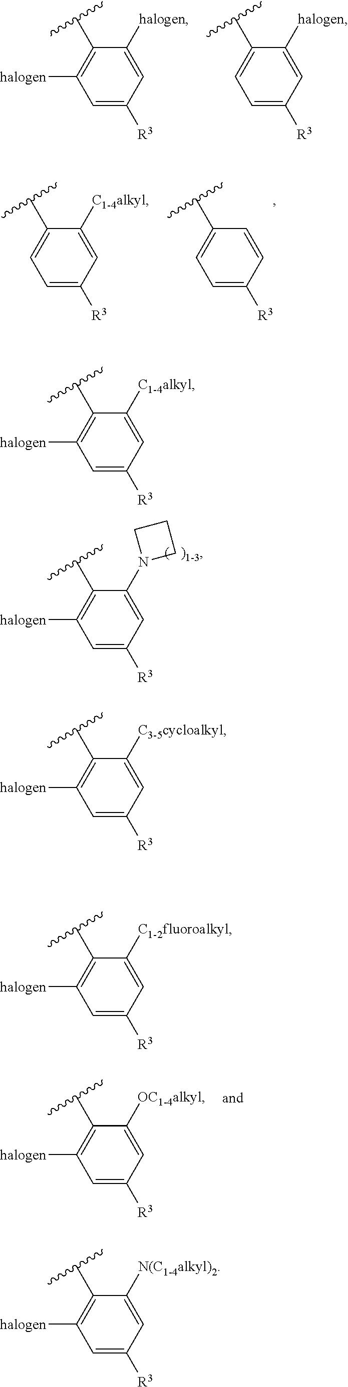

E2.1. The compound of E2, or a pharmaceutically acceptable salt thereof, wherein A¹ is selected from the group consisting of

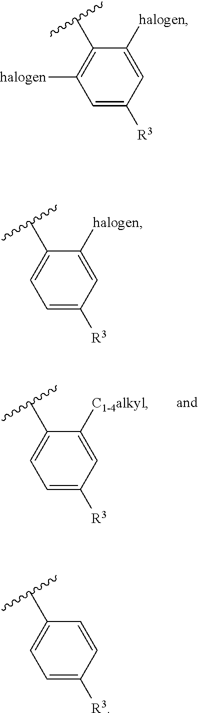

E2.2. The compound of E2, or a pharmaceutically acceptable salt thereof, wherein A¹ is selected from the group consisting of

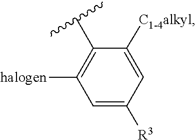

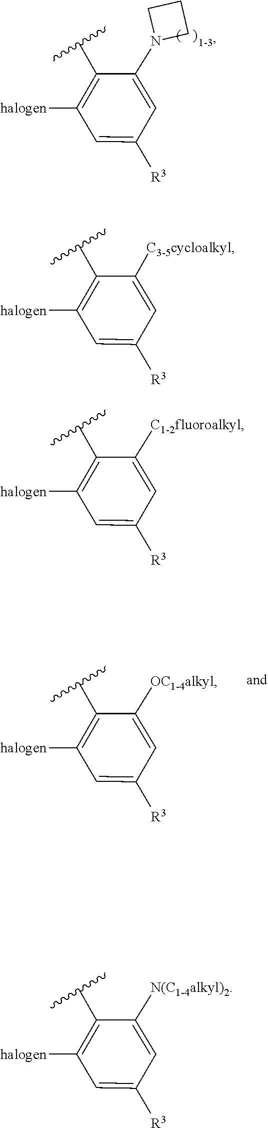

E3. The compound of any of E1-E2, or a pharmaceutically acceptable salt thereof, wherein A¹ is selected from the group consisting of

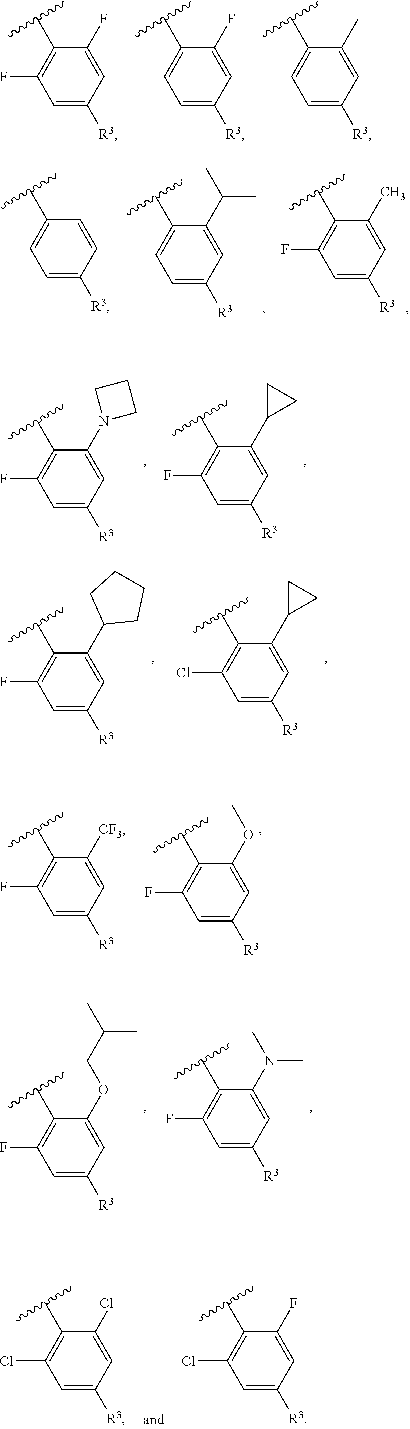

E3.1. The compound of E2.1 or E3, or a pharmaceutically acceptable salt thereof, wherein A¹ is selected from the group consisting of

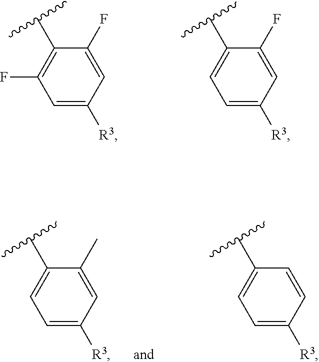

E3.2. The compound of E2.2 or E3, or a pharmaceutically acceptable salt thereof, wherein A¹ is selected from the group consisting of

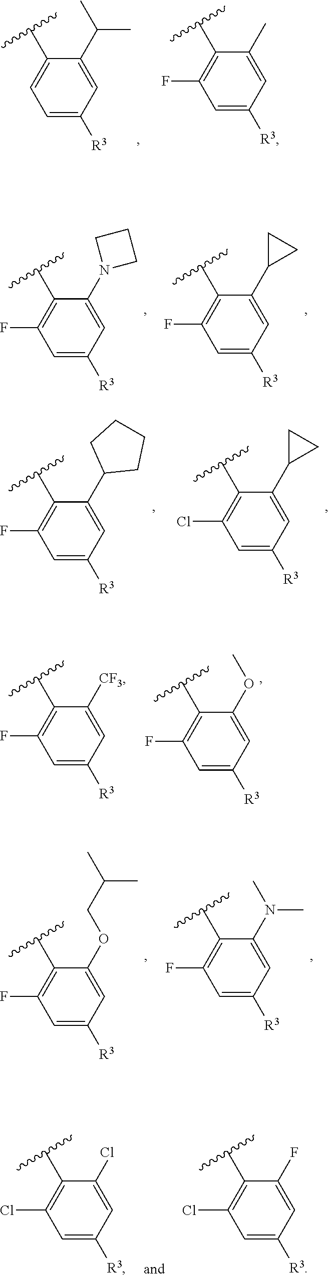

E3.3. The compound of E3.1, or a pharmaceutically acceptable salt thereof, wherein A¹ is

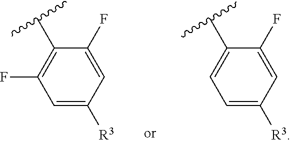

E4.1. The compound of any of E3, E3.1, or E3.3, or a pharmaceutically acceptable salt thereof, wherein A¹ is

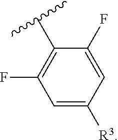

E4.2. The compound of E3 or E3.2, or a pharmaceutically acceptable salt thereof, wherein A¹ is

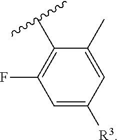

E4.3. The compound of E3 or E3.2, or a pharmaceutically acceptable salt thereof, wherein A¹ is

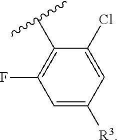

E4.4. The compound of E3 or E3.2, or a pharmaceutically acceptable salt thereof, wherein A¹ is

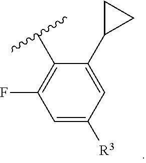

E5. The compound of any of E1-E4.4, or a pharmaceutically acceptable salt thereof, wherein the ring system of R³ is a 9-membered fused bicyclic heteroaryl containing 1-3 nitrogen atoms, the heteroaryl being unsubstituted or substituted as defined for R³ in any of E1-E1.3.

E6. The compound of E5, or a pharmaceutically acceptable salt thereof, wherein the ring system of the unsubstituted or substituted heteroaryl at R³ is selected from the group consisting of indazolyl, pyrazolo[3,4-b]pyridinyl, imidazo[1,2-a]pyridinyl, imidazo[1,5-a]pyridinyl, pyrazolo[3,4-c]pyridinyl, and pyrazolo[1,5-a]pyridinyl.

E6.1. The compound of E6, or a pharmaceutically acceptable salt thereof, wherein the ring system of the unsubstituted or substituted heteroaryl at R³ is selected from the group consisting of indazolyl, pyrazolo[3,4-b]pyridinyl, imidazo[1,2-a]pyridinyl, and imidazo[1,5-a]pyridinyl.

E6.2. The compound of E6, or a pharmaceutically acceptable salt thereof, wherein the ring system of the unsubstituted or substituted heteroaryl at R³ is pyrazolo[3,4-c]pyridinyl or pyrazolo[1,5-a]pyridinyl.

E7. The compound of E6, or a pharmaceutically acceptable salt thereof, wherein the ring system of the unsubstituted or substituted heteroaryl at R³ is selected from the group consisting of indazol-4-yl, indazol-5-yl, indazol-6-yl, pyrazolo[3,4-b]pyridin-4-yl, pyrazolo[3,4-b]pyridin-5-yl, imidazo[1,2-a]pyridin-7-yl, imidazo[1,5-a]pyridin-6-yl, 2H-pyrazolo[3,4-c]pyridin-4-yl, and pyrazolo[1,5-a]pyridin-4-yl.

E7.1. The compound of E7, or a pharmaceutically acceptable salt thereof, wherein the ring system of the unsubstituted or substituted heteroaryl at R³ is selected from the group consisting of indazol-4-yl, indazol-5-yl, indazol-6-yl, pyrazolo[3,4-b]pyridin-4-yl, pyrazolo[3,4-b]pyridin-5-yl, imidazo[1,2-a]pyridin-7-yl, and imidazo[1,5-a]pyridin-6-yl.

E7.2. The compound of E7.1, or a pharmaceutically acceptable salt thereof, wherein the ring system of the unsubstituted or substituted heteroaryl at R³ is selected from the group consisting of 1H-indazol-4-yl, 1H-indazol-5-yl, 2H-indazol-4-yl, 2H-indazol-5-yl, 2H-indazol-6-yl, 2H-pyrazolo[3,4-b]pyridin-4-yl, and 2H-pyrazolo[3,4-b]pyridin-5-yl.

E7.3. The compound of E7, or a pharmaceutically acceptable salt thereof, wherein the ring system of the unsubstituted or substituted heteroaryl at R³ is 2H-pyrazolo[3,4-c]pyridin-4-yl or pyrazolo[1,5-a]pyridin-4-yl.

E8. The compound of E5, or a pharmaceutically acceptable salt thereof, wherein R³ is selected from the group consisting of

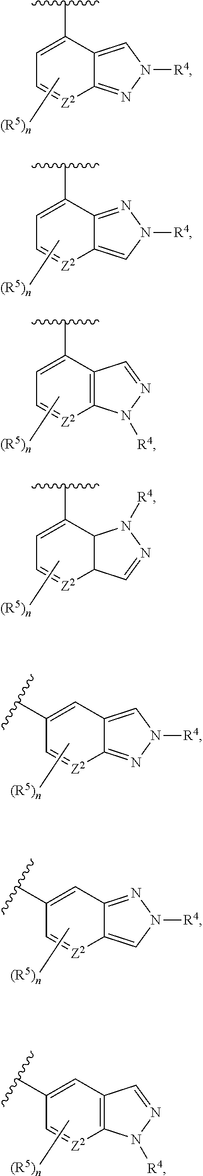

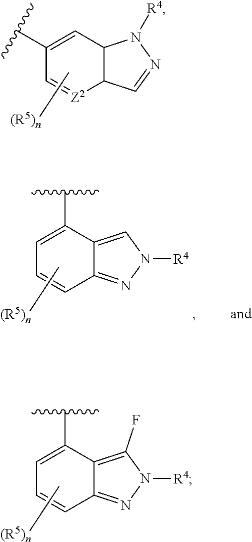

Z² is N or CR^{5a};
R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R⁵, at each occurrence, is independently selected from the group consisting of halogen, C₁₋₄alkyl, C₁₋₄haloalkyl, —OC₁₋₄alkyl, —OC₁₋₄deuterioalkyl, and —OC₁₋₄haloalkyl;
R^{5a} is H or R⁵; and
n is 0, 1, or 2.

E8.1. The compound of E8, or a pharmaceutically acceptable salt thereof, wherein R³ is selected from the group consisting of

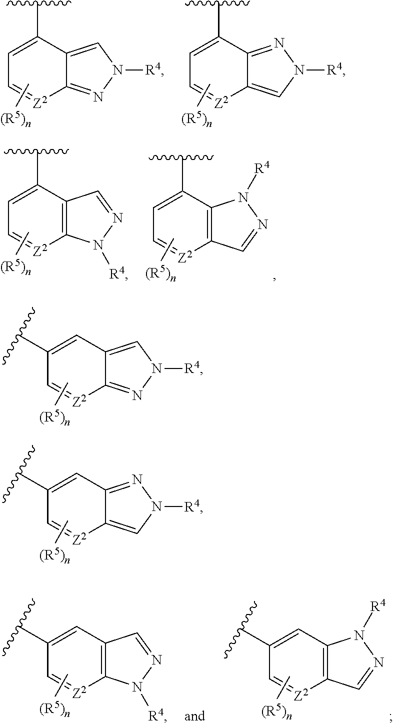

Z² is N or CR^{5a};
R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R⁵, at each occurrence, is independently selected from the group consisting of halogen, C₁₋₄alkyl, C₁₋₄haloalkyl, —OC₁₋₄alkyl, —OC₁₋₄deuterioalkyl, and —OC₁₋₄haloalkyl;
R^{5a} is H or R⁵; and
n is 0, 1, or 2.

E8.2. The compound of E8, or a pharmaceutically acceptable salt thereof, wherein R³ is

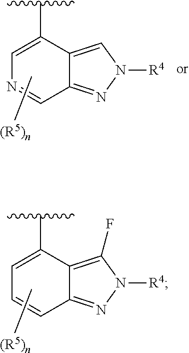

R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R⁵, at each occurrence, is independently selected from the group consisting of halogen, C₁₋₄alkyl, C₁₋₄haloalkyl, —OC₁₋₄alkyl, —OC₁₋₄deuterioalkyl, and —OC₁₋₄haloalkyl;
R^{5a} is H or R⁵; and
n is 0, 1, or 2.

In the depiction of “(R⁵)n-” in E8-E8.2 as a substituent on R³, any R⁵ group, when present, is substituted on the 6-membered ring of the fused bicyclic ring system of R³. The R⁵ group present when Z² is CR^{5a} and R^{5a} is R⁵ is independent of the 0, 1, or 2 R⁵ groups of “(R⁵)n-”, such that R³ may be substituted on its 6-membered ring with up to three R⁵ groups.

E8.3. The compound of E8.1, or a pharmaceutically acceptable salt thereof, wherein when Z² is CR^{5a}; R^{5a} is R⁵; and n is 0, then R³ has one R⁵ substituent

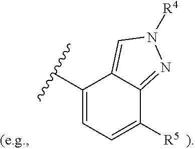

E8.4. The compound of E8.1, or a pharmaceutically acceptable salt thereof, wherein when Z² is CR^{5a}; R^{5a} is R⁵; and n is 1, then R³ has two R⁵ substituents

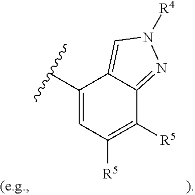

E8.5. The compound of E8.1, or a pharmaceutically acceptable salt thereof, wherein when Z² is CR^{5a}; R^{5a} is R⁵; and n is 2, then R³ has three R⁵ substituents

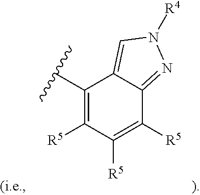

E9. The compound of any of E8-E8.4, or a pharmaceutically acceptable salt thereof, wherein n is 0 or 1.

E10. The compound of any of E1-E9, or a pharmaceutically acceptable salt thereof, wherein R³ is selected from the group consisting of

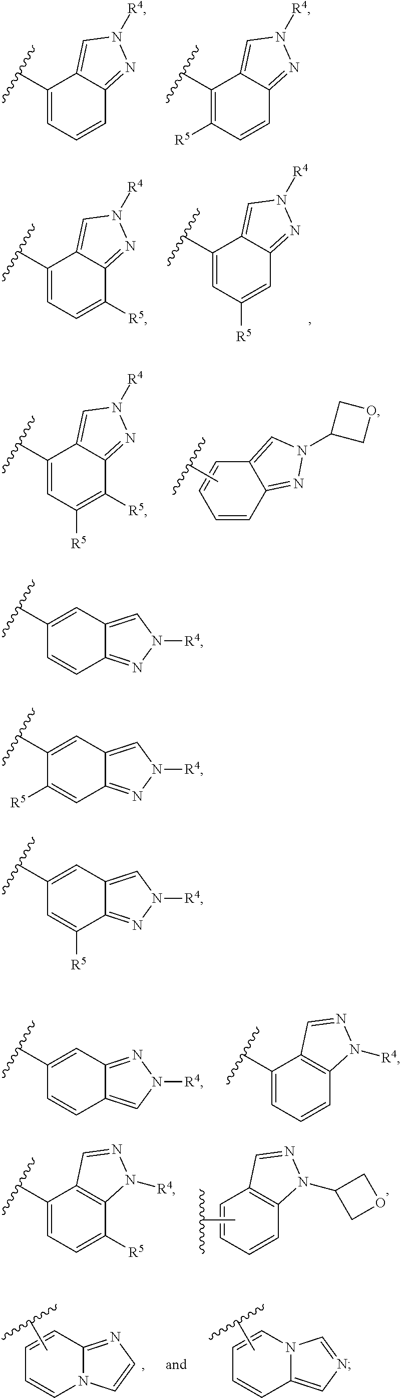

or
R³ is

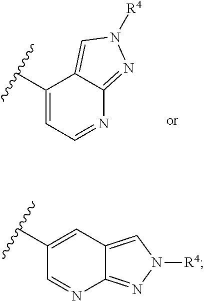

or
R³ is selected from the group consisting of

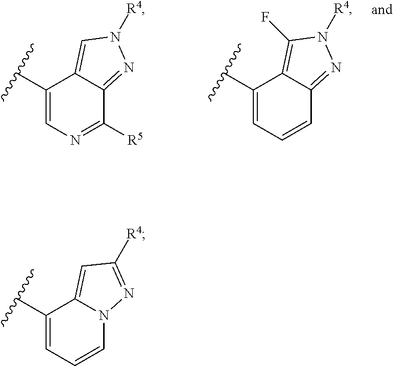

R⁴ is selected from the group consisting of H, methyl, ethyl, isopropyl, CH₂F, CHF₂, CD₃, and CD₂CD₃; and
R⁵, at each occurrence, is independently selected from the group consisting of fluoro, chloro, methyl, CHF₂, CF₃, OCH₃, OCD₃, OCHF₂, and OCF₃.

E10.1. The compound of E10, or a pharmaceutically acceptable salt thereof, wherein R³ is selected from the group consisting of

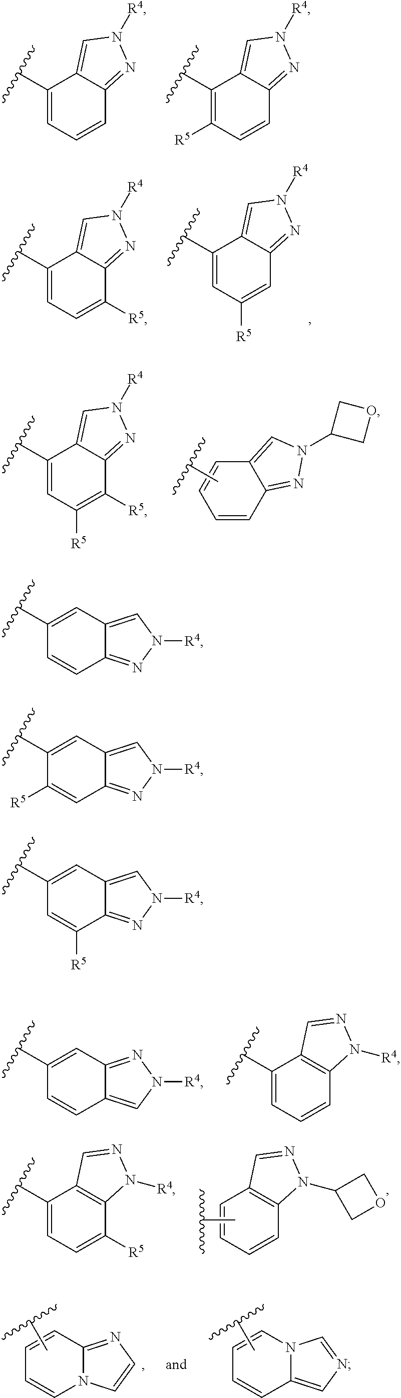

wherein
R⁴ is selected from the group consisting of H, methyl, ethyl, isopropyl, CH₂F, CHF₂, CD₃, and CD₂CD₃; and
R⁵, at each occurrence, is independently selected from the group consisting of fluoro, chloro, methyl, CF₃, and OCH₃.

E10.2. The compound of E10, or a pharmaceutically acceptable salt thereof,

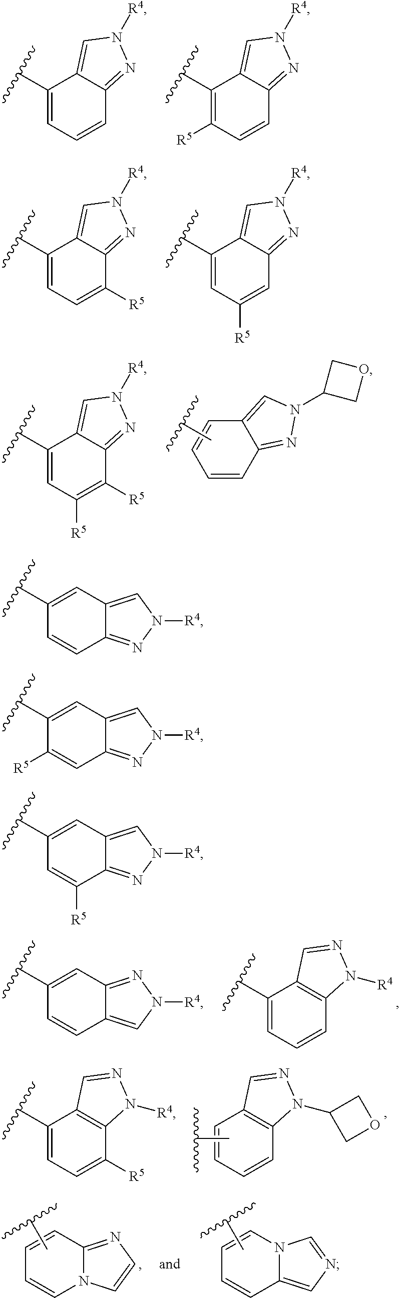

or
R³ is

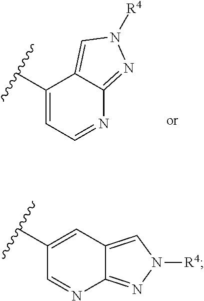

wherein
R⁴ is selected from the group consisting of H, methyl, ethyl, isopropyl, CH₂F, CHF₂, CD₃, and CD₂CD₃; and
R⁵, at each occurrence, is independently selected from the group consisting of fluoro, chloro, methyl, CF₃, and OCH₃.

E10.3. The compound of E10.2, or a pharmaceutically acceptable salt thereof, wherein R³ is selected from the group consisting of

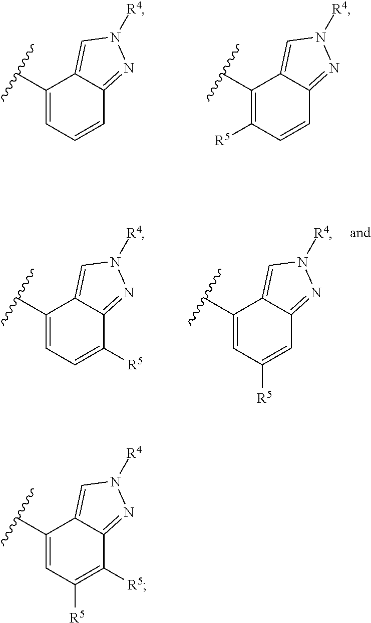

or
R³ is

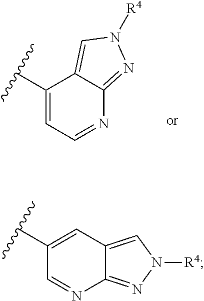

wherein
R⁴ is selected from the group consisting of methyl, ethyl, CD₃, and CD₂CD₃; and
R⁵, at each occurrence, is independently selected from the group consisting of fluoro, chloro, methyl, CF₃, and OCH₃.

E11. The compound of any of E1-E6.1 or E7-E7.2, or a pharmaceutically acceptable salt thereof, wherein:
R³ is

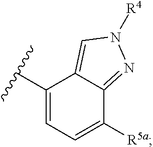

R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R^{5a} is H or R⁵; and
R⁵ is selected from the group consisting of halogen, C₁₋₆alkyl, C₁₋₆deuterioalkyl, C₁₋₆haloalkyl, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl.

E11.1. The compound of E11, or a pharmaceutically acceptable salt thereof, wherein R^{5a} is R⁵.

E11.2. The compound of E11 or E11.1, or a pharmaceutically acceptable salt thereof, wherein R⁵ is selected from the group consisting of halogen, C₁₋₄alkyl, C₁₋₂fluoroalkyl, —OH, —OR^{3a}, —OC₁₋₂fluoroalkyl, —SC₁₋₄alkyl, —S(O)(NH)C₁₋₄alkyl, and C₃₋₅cycloalkyl.

E11.3. The compound of any of E11-E11.2, or a pharmaceutically acceptable salt thereof, wherein:
R^{3a} is C₁₋₄alkyl or C₁₋₄deuterioalkyl, each independently of each other unsubstituted or substituted with a substituent selected from the group consisting of —OR^{3b}, —N(R^{3b})₂, —SR^{3b} and —SO₂R^{3c}; or
R^{3a} is selected from the group consisting of —C₃₋₆cycloalkyl, —CH₂—C₃₋₆cycloalkyl, —CH₂-phenyl, -heterocyclyl, —CH₂-heterocyclyl, wherein each heterocyclyl is a 4 to 7-membered heterocyclyl containing 1-2 heteroatoms independently selected from the group consisting of O, N, and S, wherein each —C₃₋₆cycloalkyl and -heterocyclyl is unsubstituted or substituted with a first substituent selected from the group consisting of halogen, cyano, C₁₋₄alkyl, C₁₋₄deuterioalkyl, C₁₋₂fluoroalkyl, C₃₋₆cycloalkyl, oxo, —OR^{3b}, —N(R^{3b})₂, —C(O)OR^{3b}, —SO₂R³, and C(O)R^{3b} and optionally further substituted with 1-3 substituents independently selected from the group consisting of halogen and C₁₋₄alkyl;
R^{3b}, at each occurrence, is independently selected from the group consisting of C₁₋₄alkyl and C₃₋₄cycloalkyl; and
R³, is C₁₋₄alkyl or C₃₋₄cycloalkyl.

E11.4. The compound of E11.3, or a pharmaceutically acceptable salt thereof, C₁₋₄deuterioalkyl wherein R³ is selected from the group consisting of

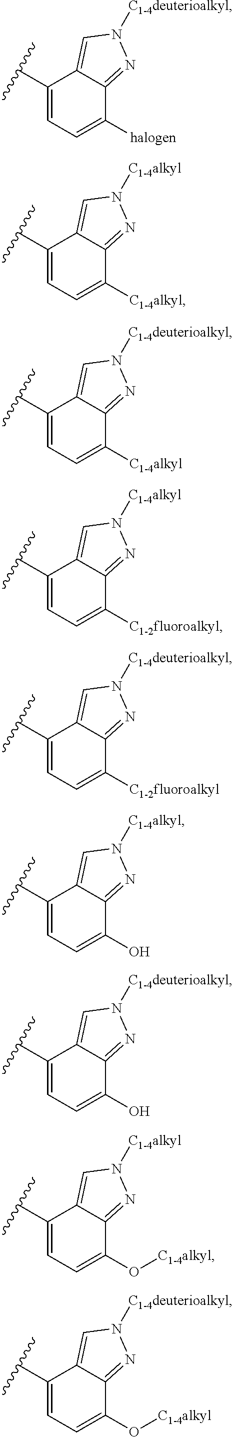

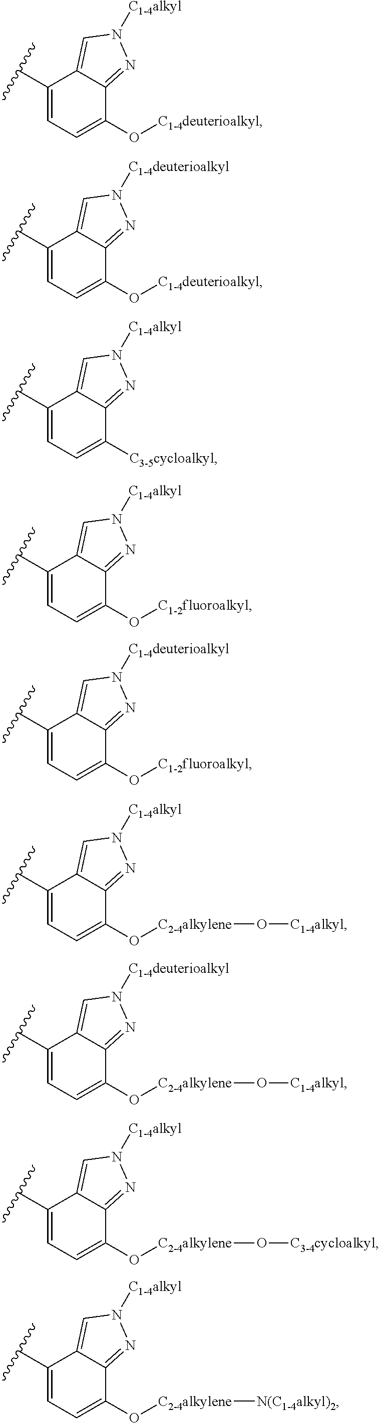

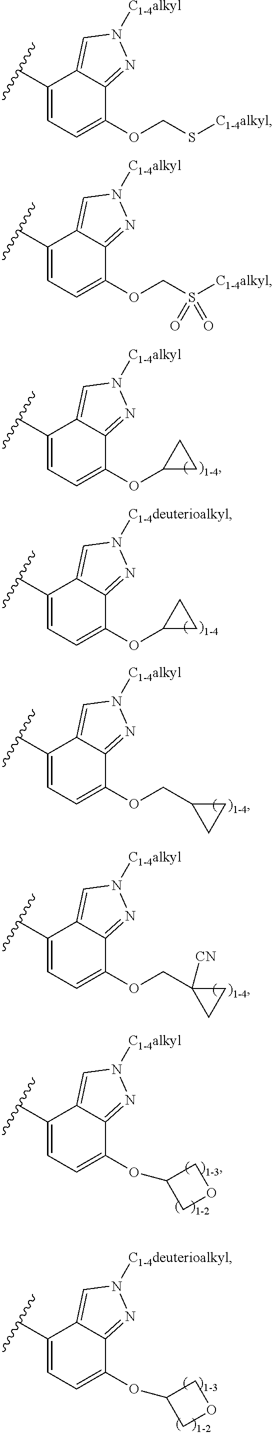

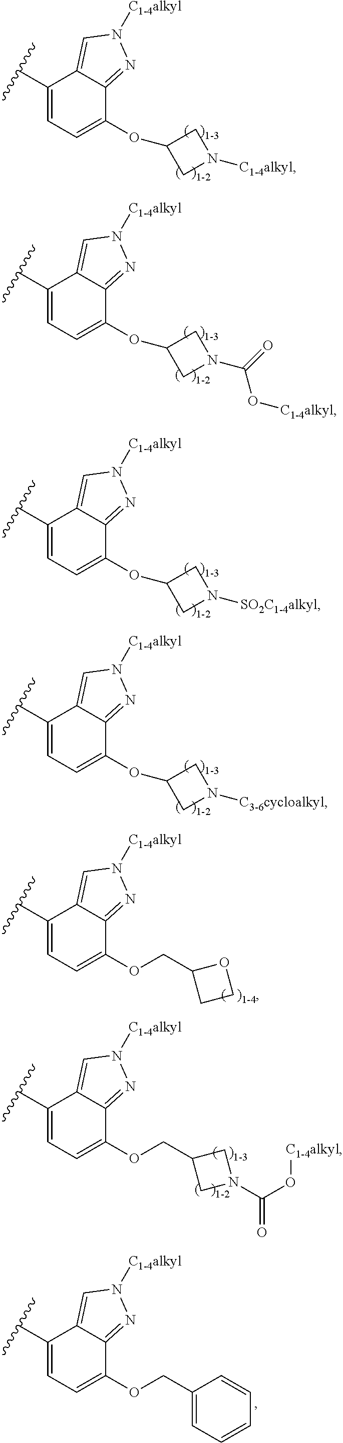

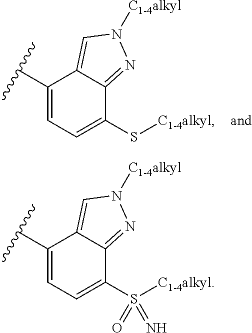

E11.5. The compound of E11.4, or a pharmaceutically acceptable salt thereof, wherein R³ is selected from the group consisting of

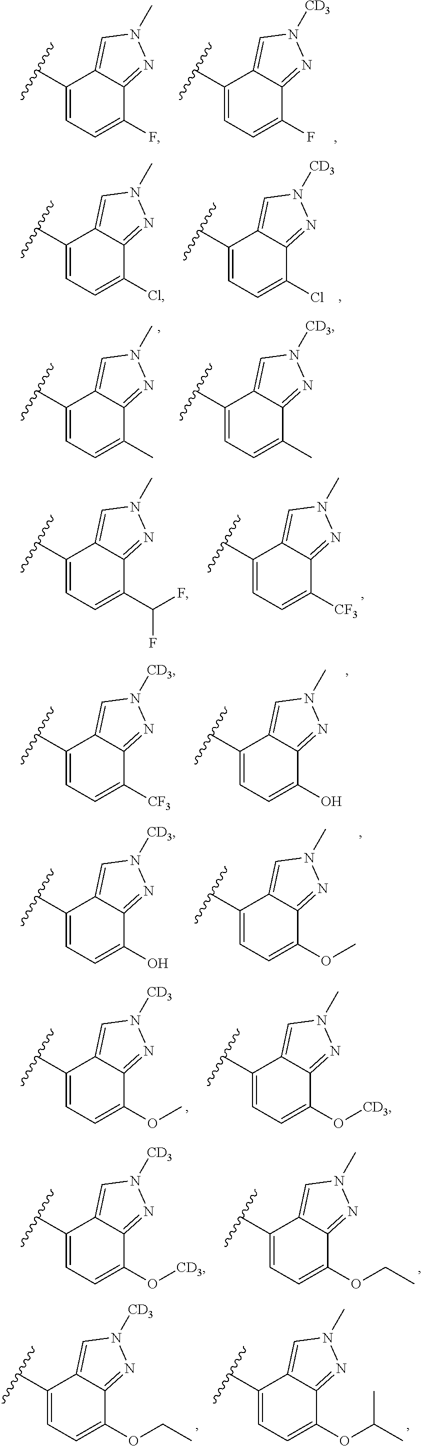

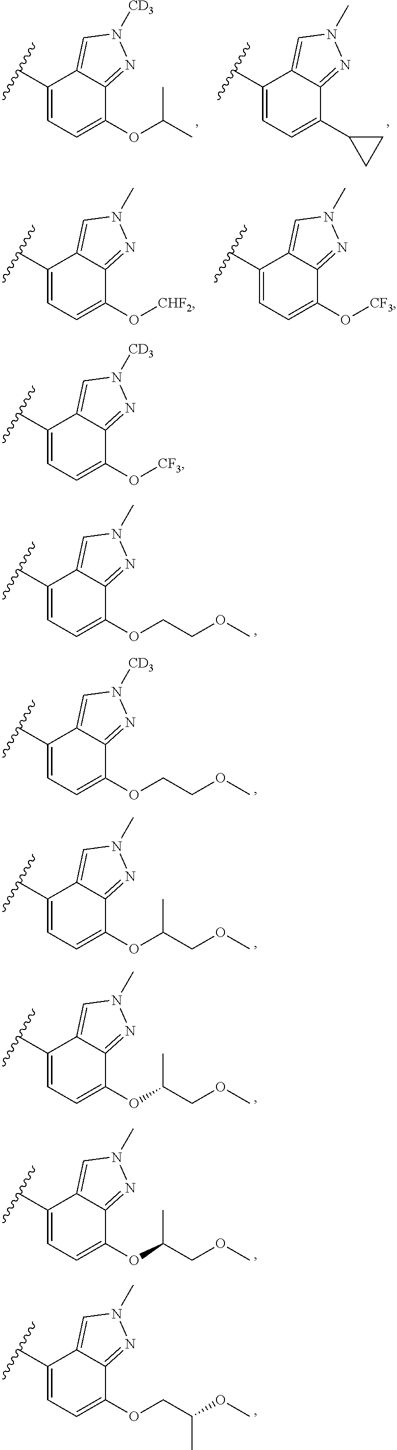

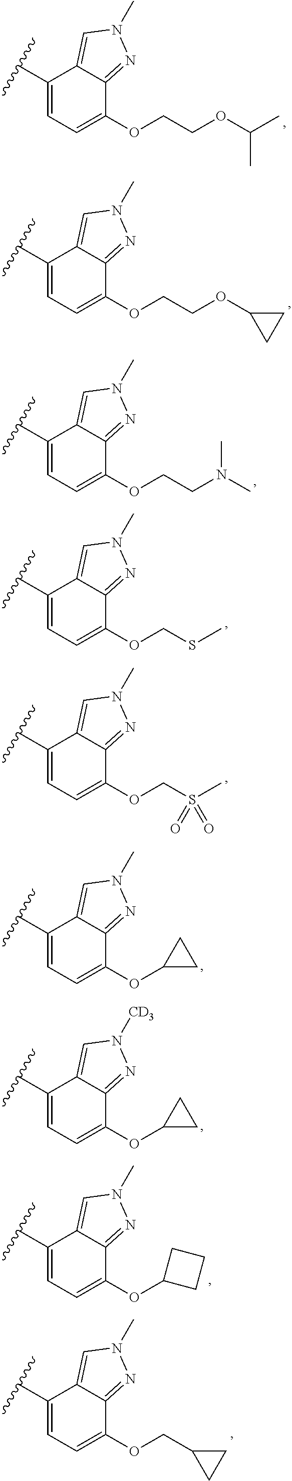

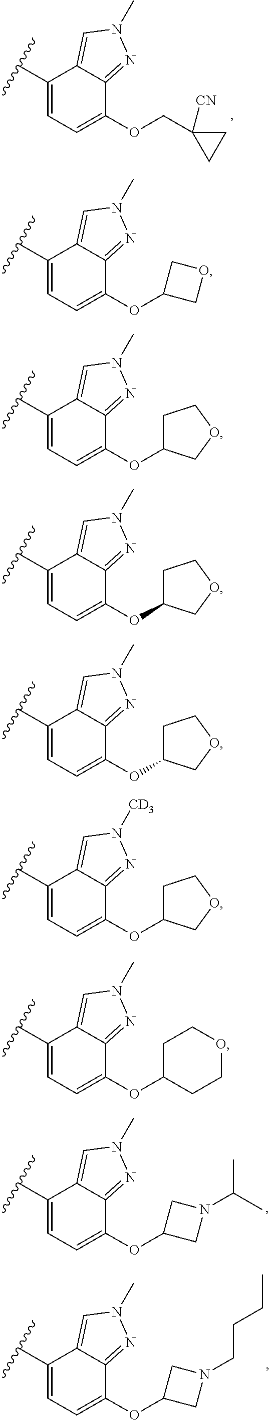

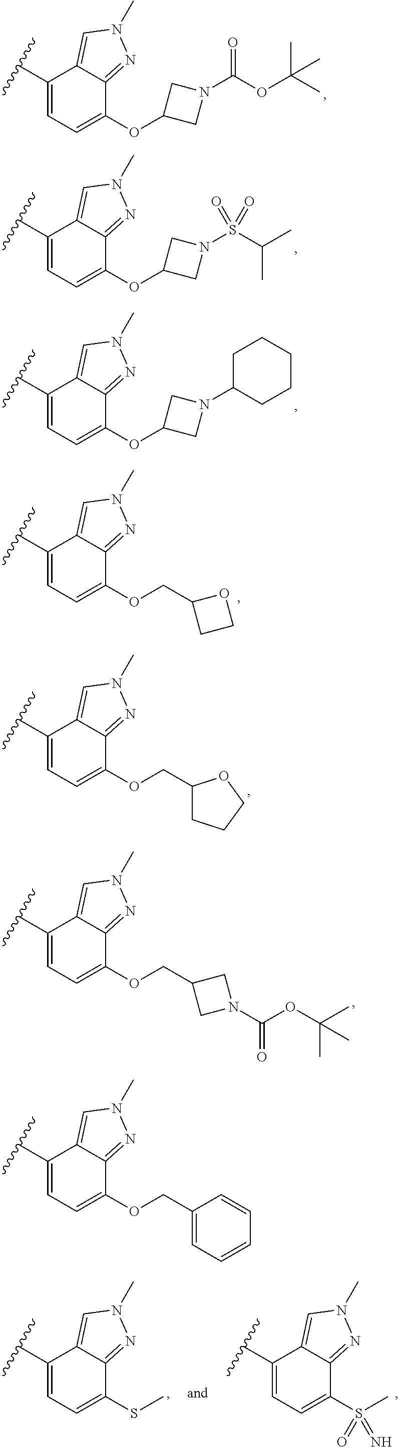

E11.6. The compound of E11, or a pharmaceutically acceptable salt thereof, wherein R^{5a} is H.

E11.7. The compound of E11.6, or a pharmaceutically acceptable salt thereof, wherein R³ is

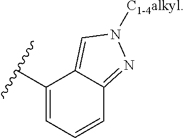

E11.8. The compound of E11.7, or a pharmaceutically acceptable salt thereof, wherein R³ is

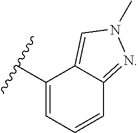 [SMILES: *c1cccc2nn(C)cc12]

E11.9. The compound of E11.6 or E11.7, or a pharmaceutically acceptable salt C₁₋₄deuterioalkyl thereof, wherein R³ is

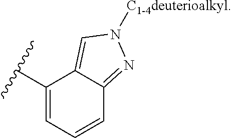

E11.10. The compound of E11.8 or E11.9, or a pharmaceutically acceptable salt thereof, wherein R³ is

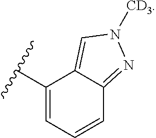 [SMILES: *c1cccc2nn(C([2H])([2H])[2H])cc12]

E12. The compound of any of E8-E9, E11-E11.3, or E11.6, or a pharmaceutically acceptable salt thereof, wherein R⁴ is selected from the group consisting of C₁₋₄alkyl, C₁₋₄deuterioalkyl, C₁₋₂fluoroalkyl, C₃₋₄cycloalkyl, —C₁₋₃alkylene-C₃₋₄cycloalkyl, and oxetan-3-yl.

E13. The compound of any of E8-E10.2, E11-E11.3, E11.6, or E12, or a pharmaceutically acceptable salt thereof, wherein R⁴ is selected from the group consisting of methyl, ethyl, isopropyl, CH₂F, CHF₂, and CD₃.

E13.1. The compound of any of E7-E10.3, E10.6, or E11-E12, or a pharmaceutically acceptable salt thereof, wherein R⁴ is methyl.

E13.2. The compound of any of E8-E11.3, E11.6, or E12-E13.1, or a pharmaceutically acceptable salt thereof, wherein R⁴ is CD₃.

E14. The compound of any of E1-E10, or a pharmaceutically acceptable salt thereof, wherein R³ is selected from the group consisting of

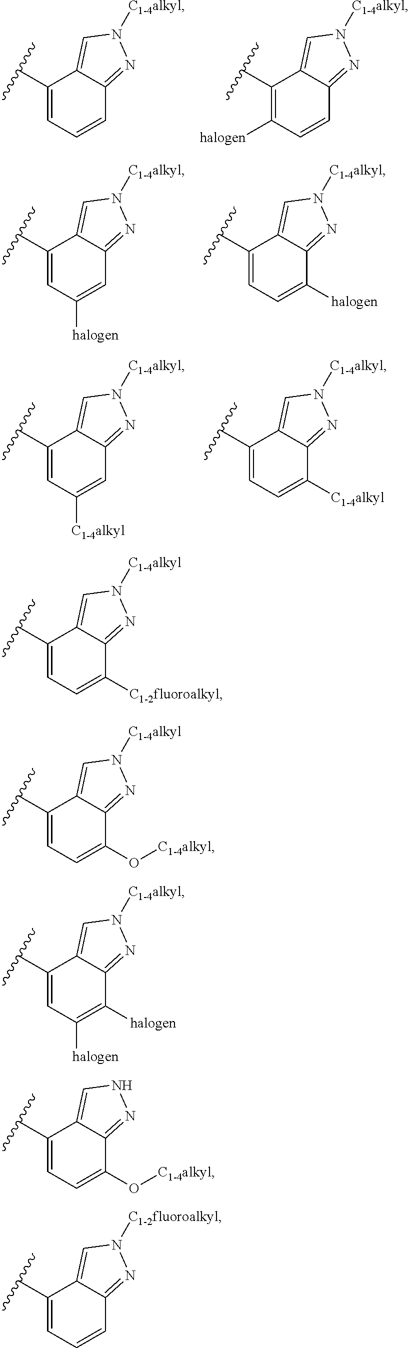

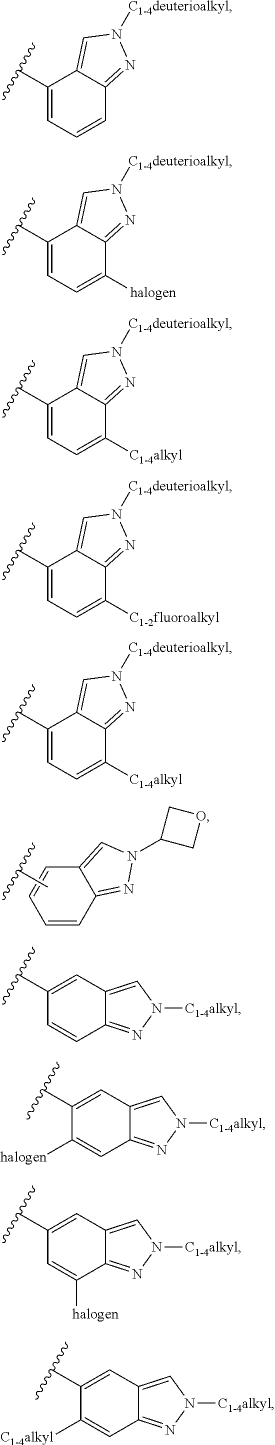

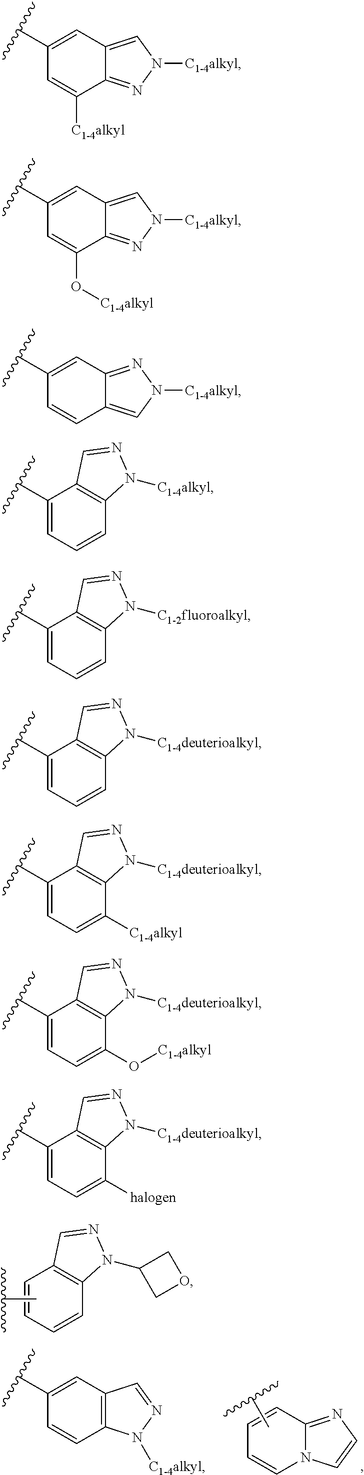

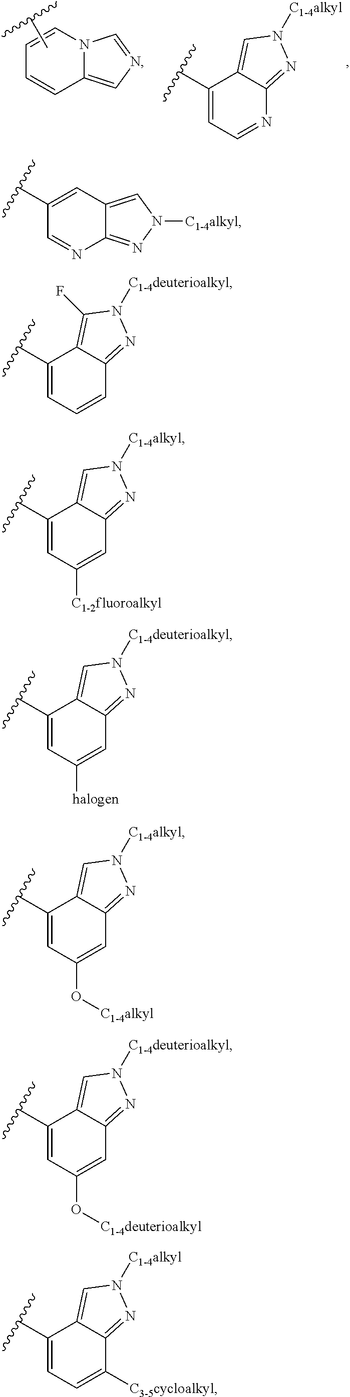

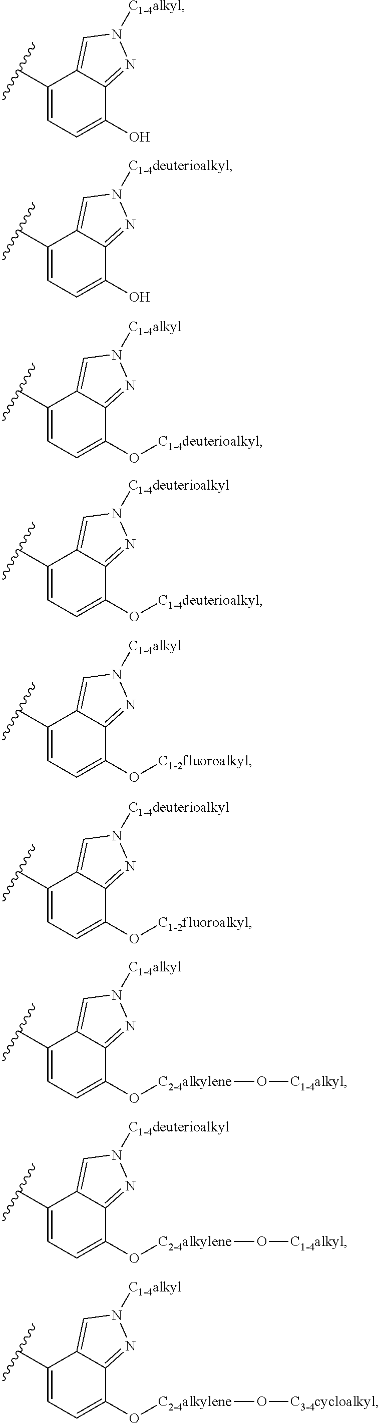

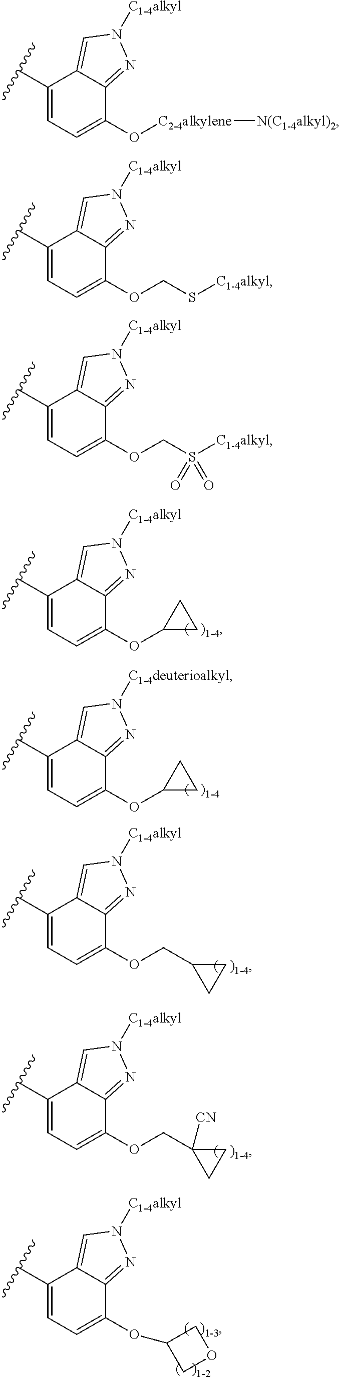

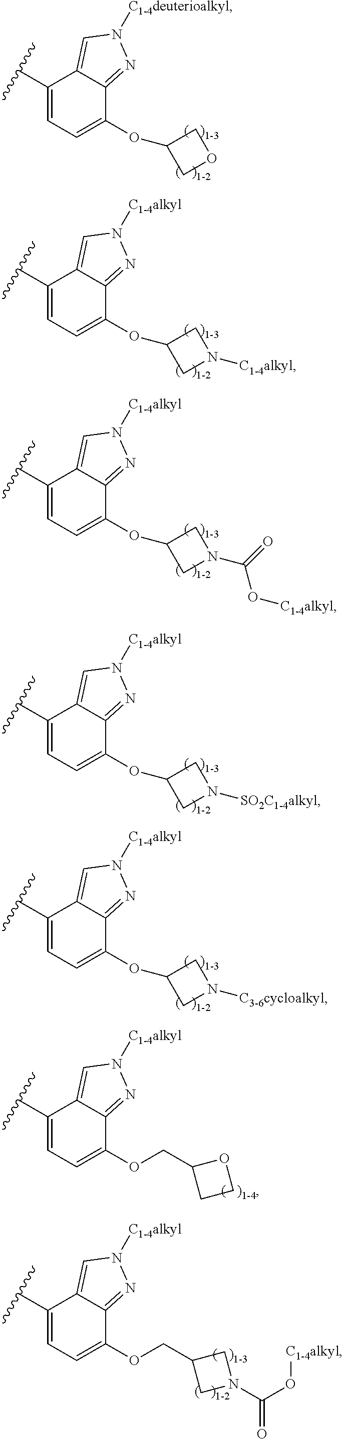

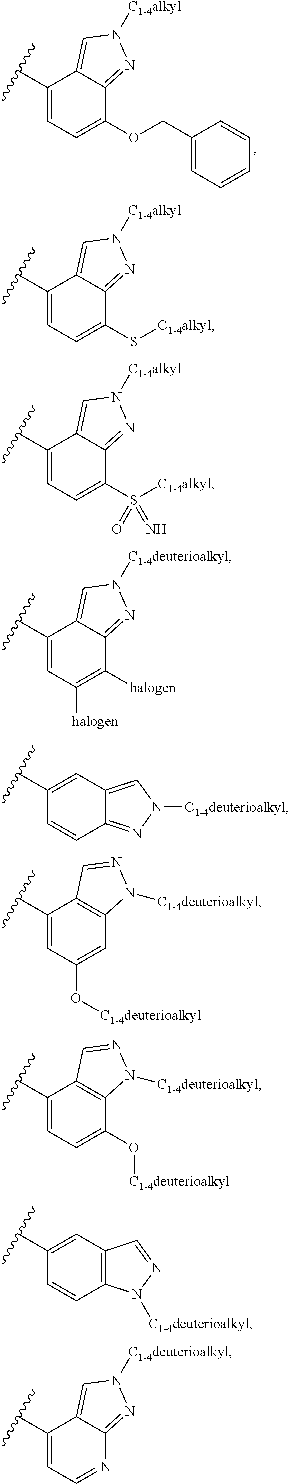

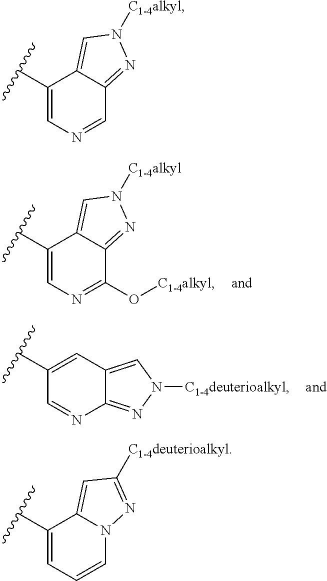

E14.1. The compound of E14, or a pharmaceutically acceptable salt thereof, wherein R³ is selected from the group consisting of

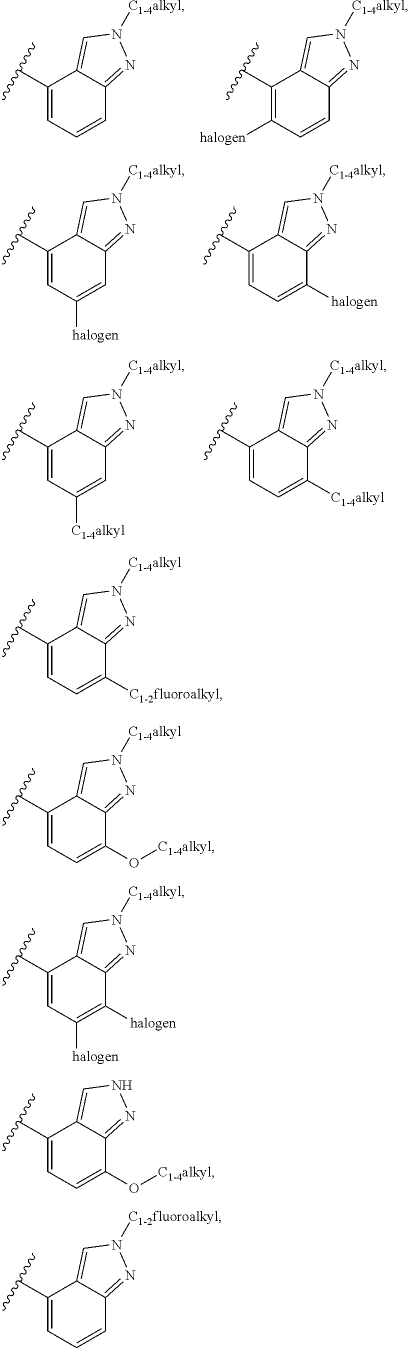

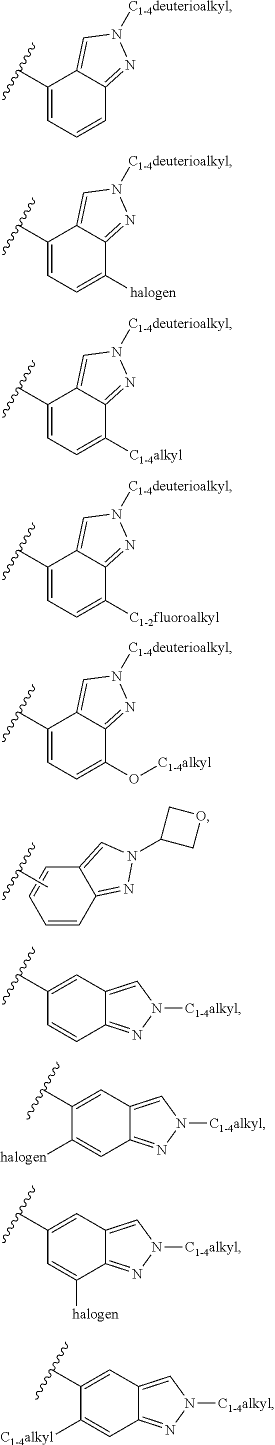

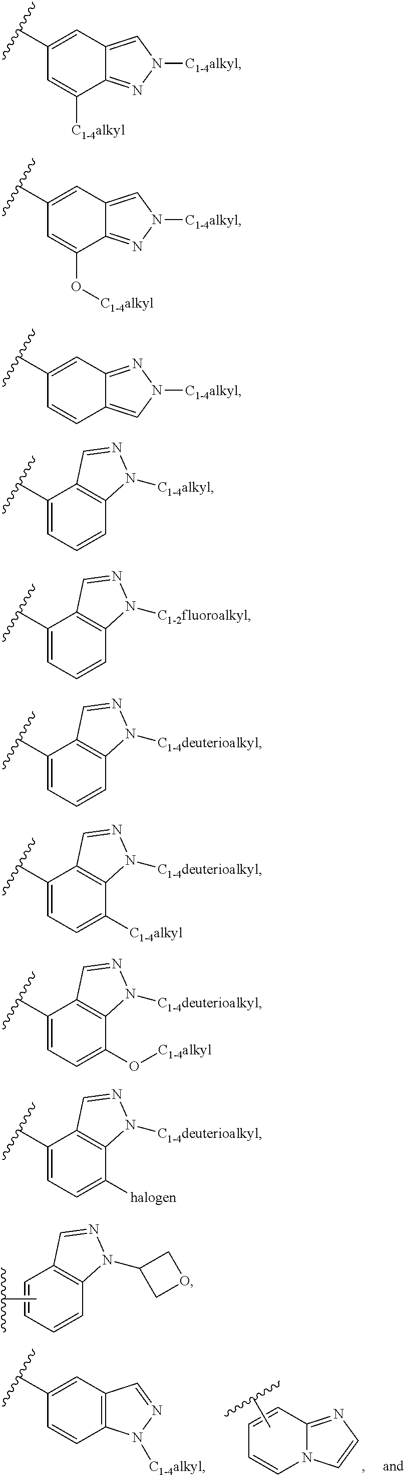

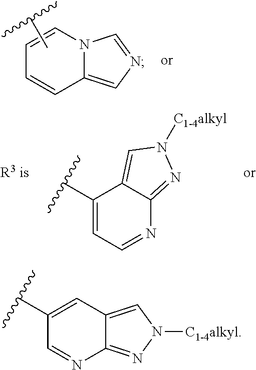

E14.2. The compound of E14, or a pharmaceutically acceptable salt thereof, wherein R³ is selected from the group consisting of

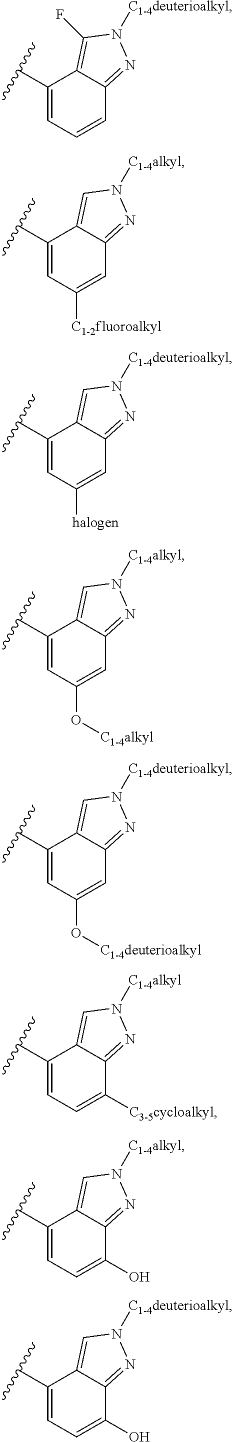

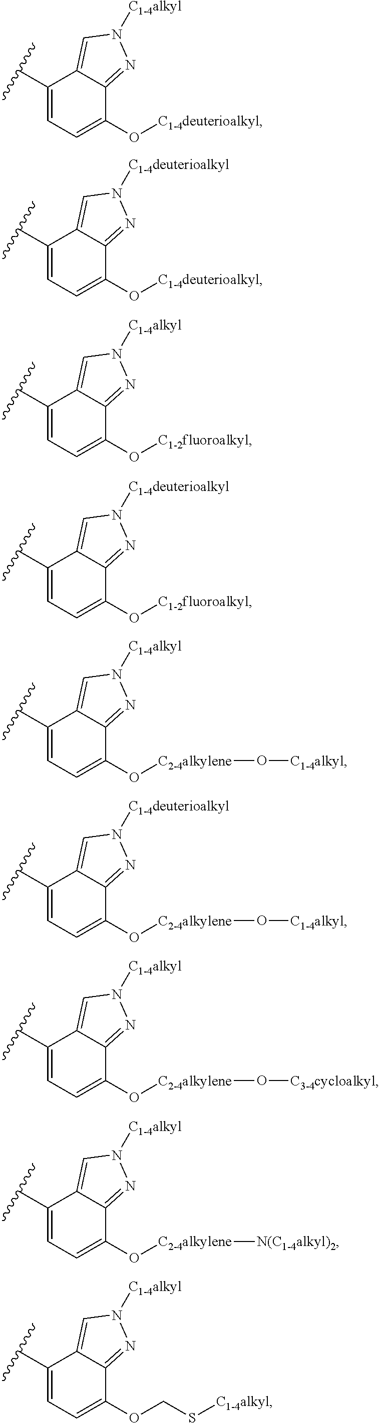

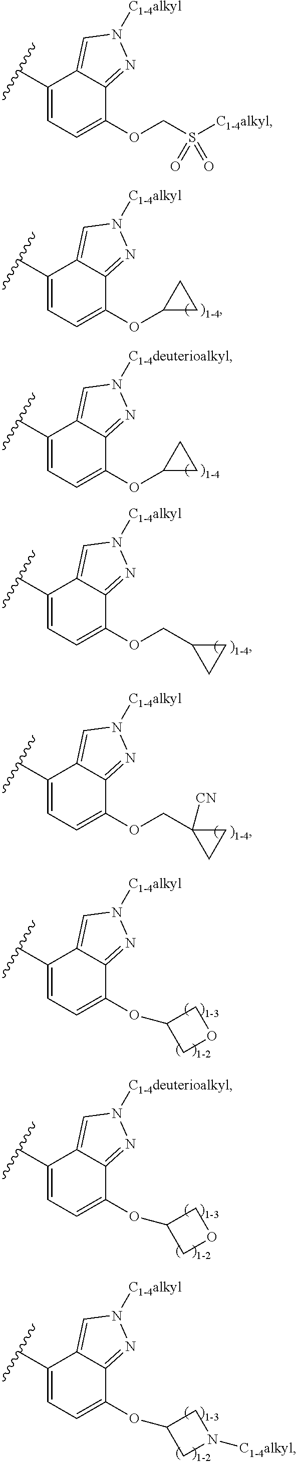

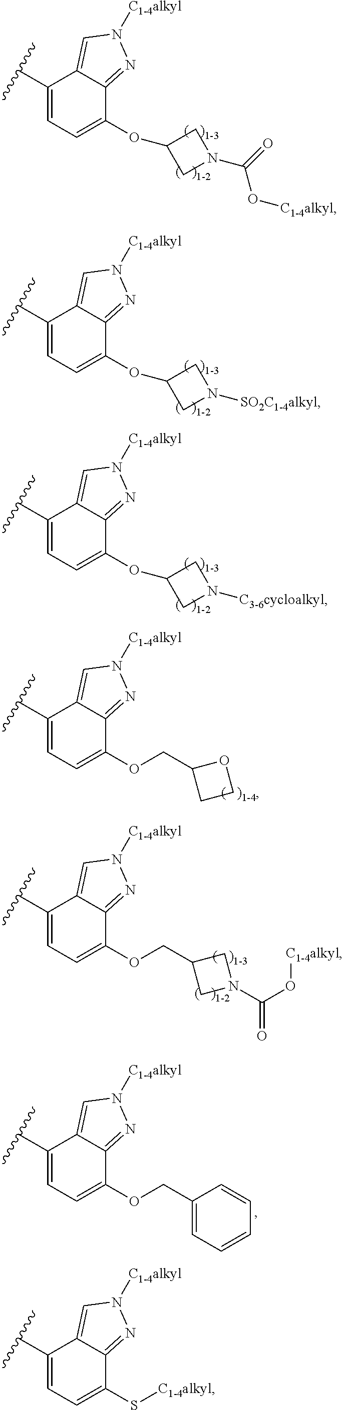

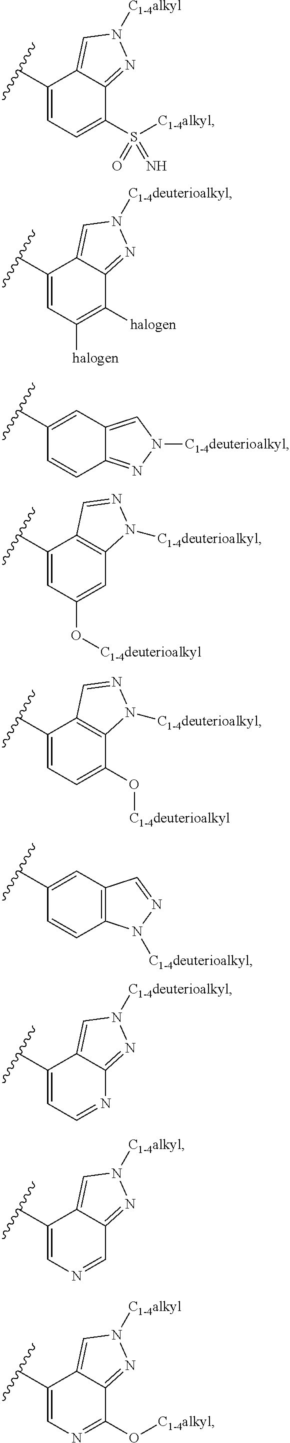

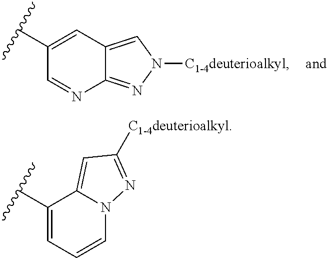

E15. The compound of any of E1-E10 or E14, or a pharmaceutically acceptable salt thereof, wherein R³ is selected from the group consisting of

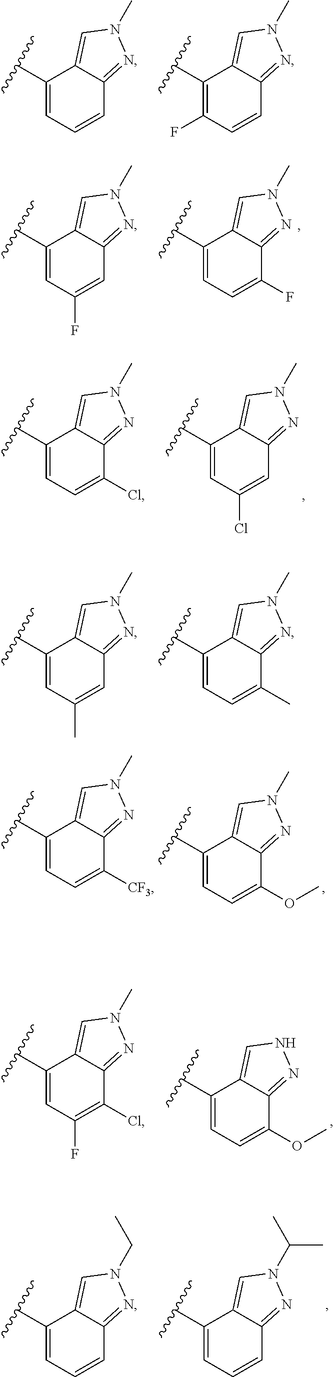

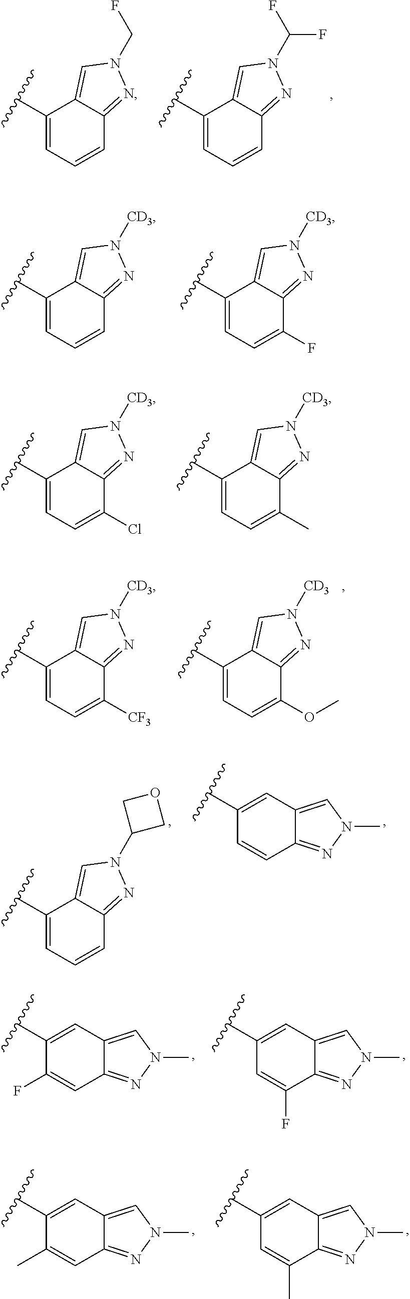

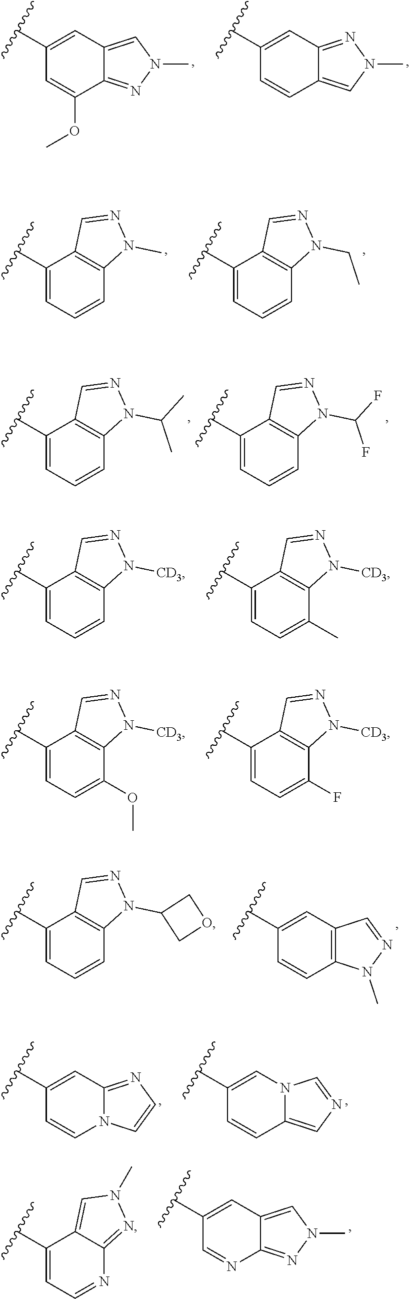

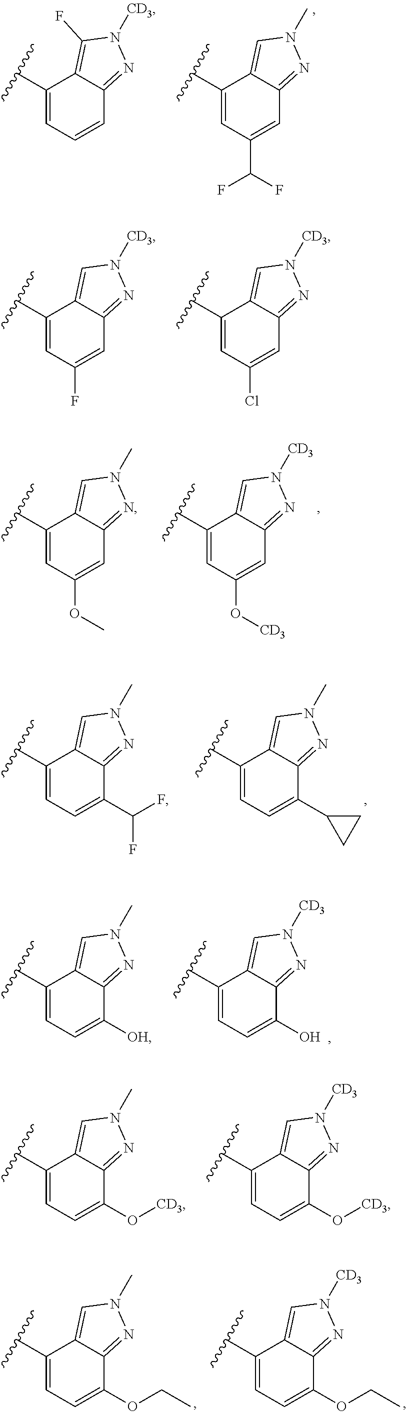

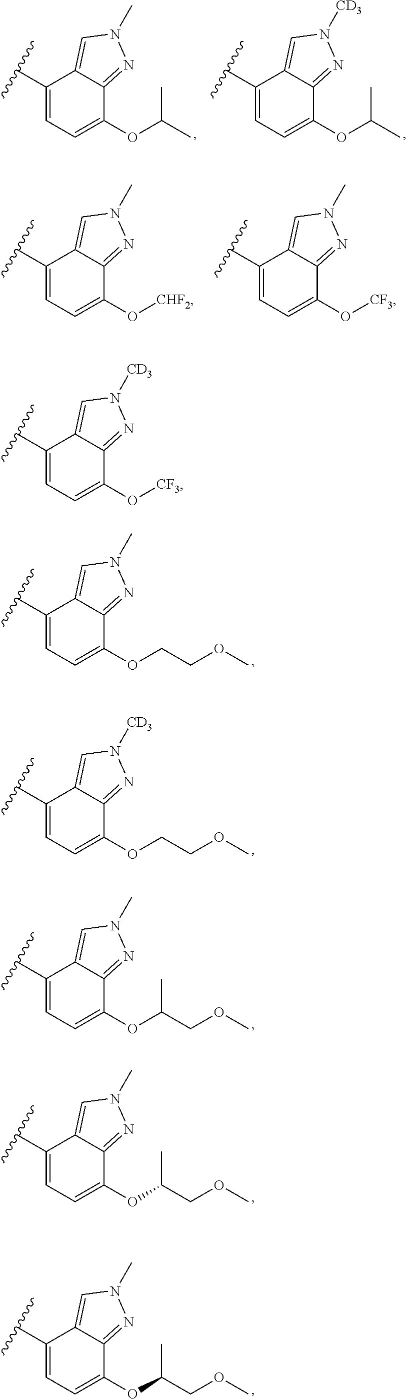

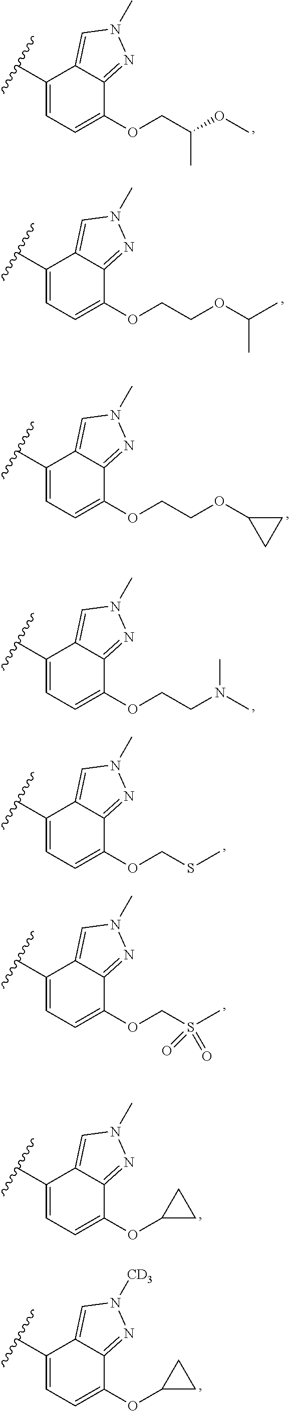

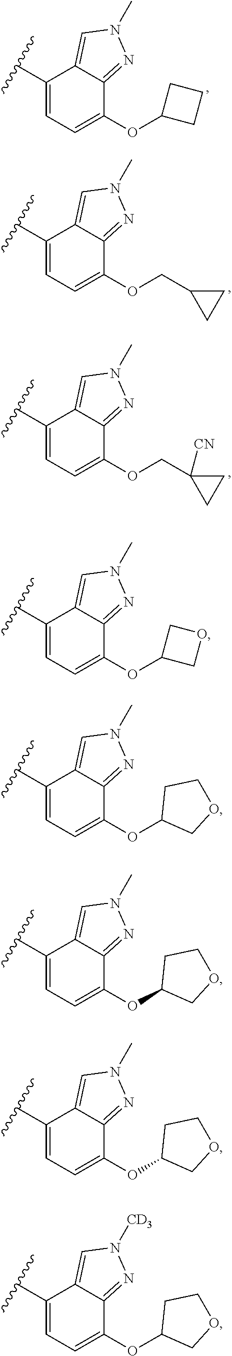

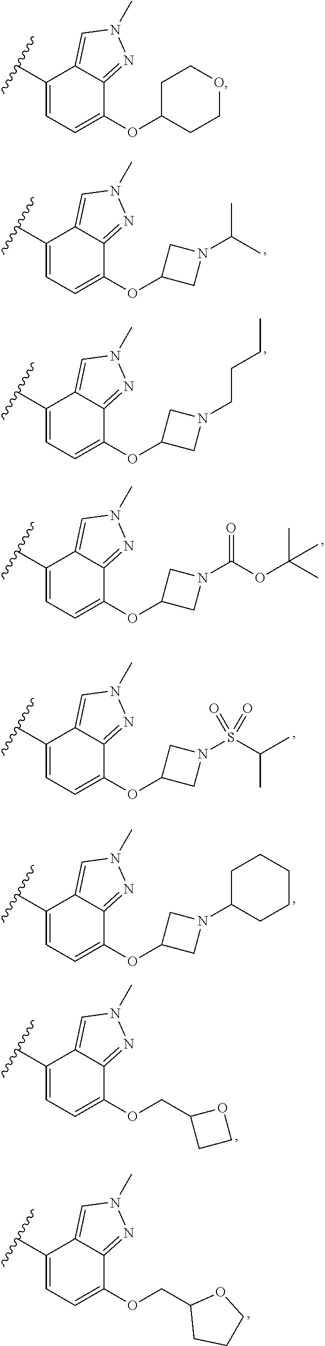

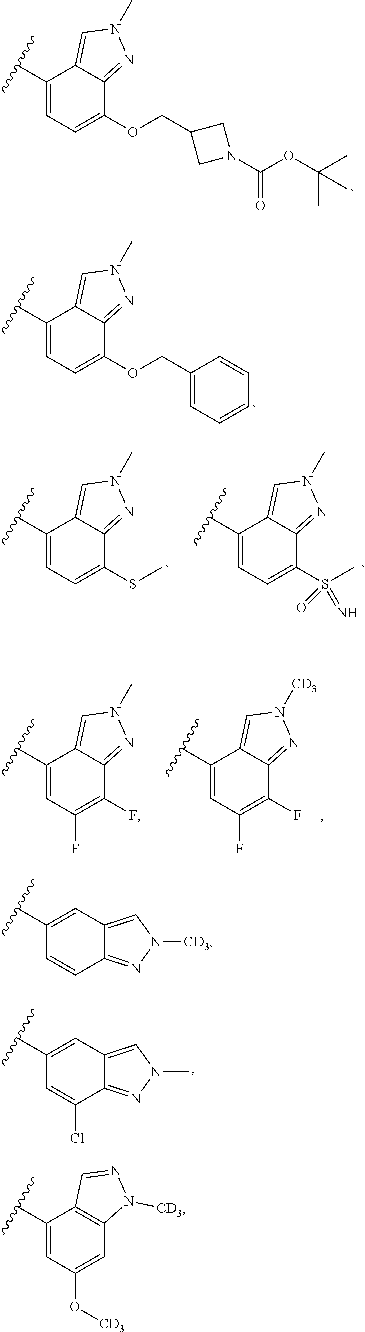

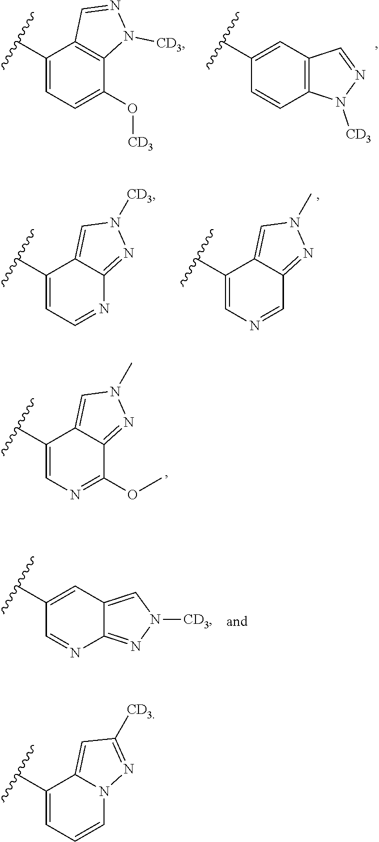

E15.1. The compound of E15, or a pharmaceutically acceptable salt thereof, wherein R³ is selected from the group consisting of

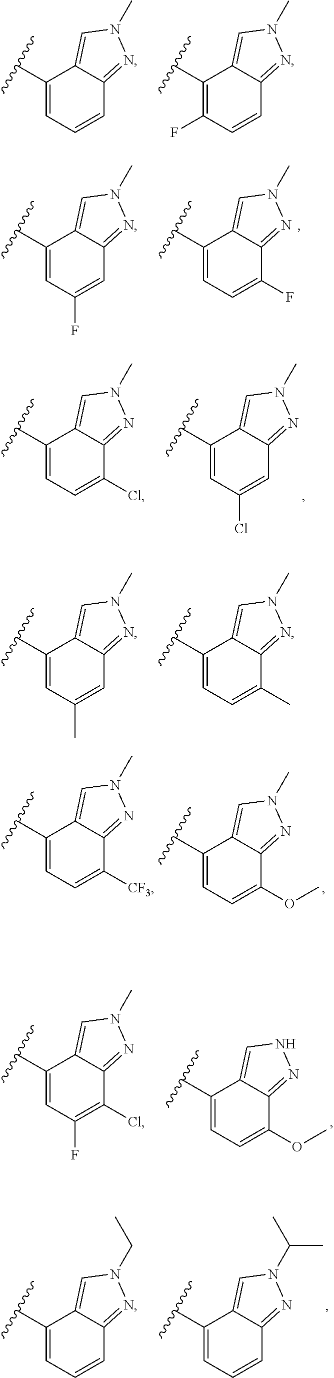

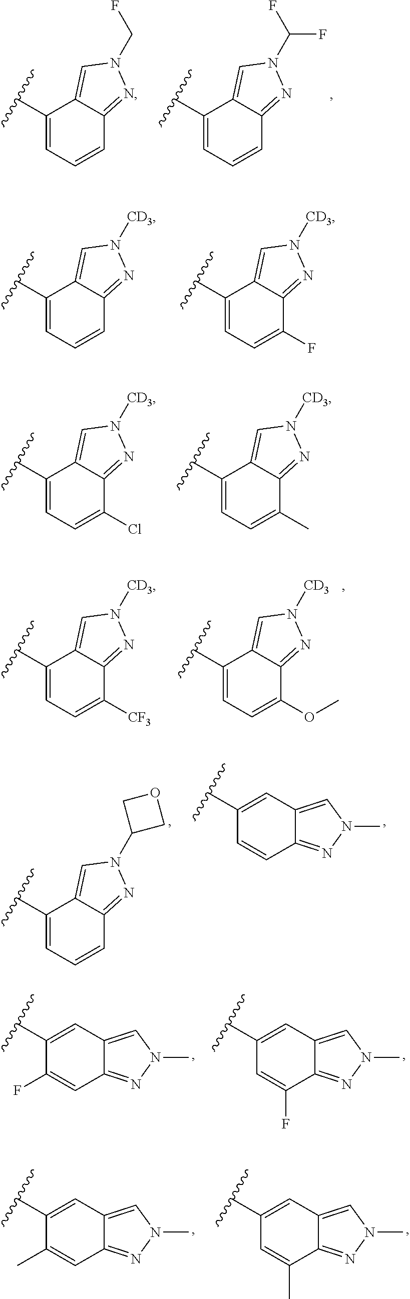

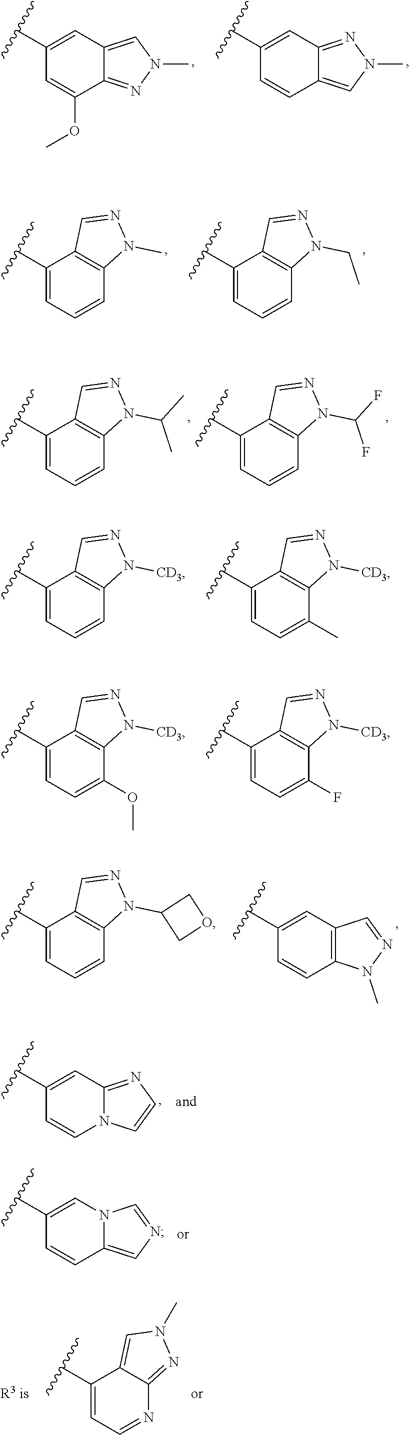

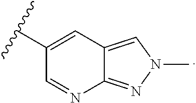 [SMILES: *c1cnc2nn(C)cc2c1]

E15.2. The compound of E15, or a pharmaceutically acceptable salt thereof, wherein R³ is selected from the group consisting of

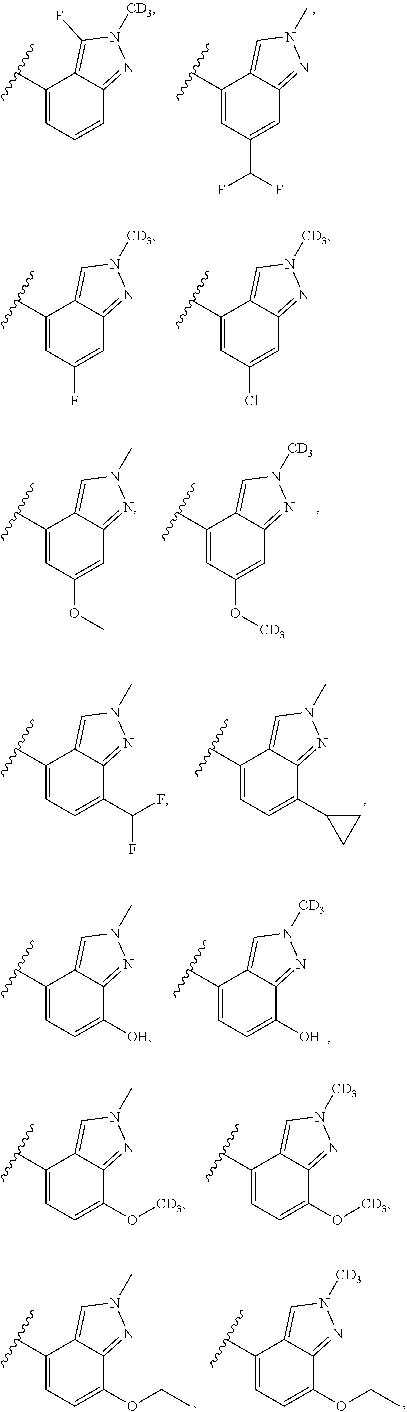

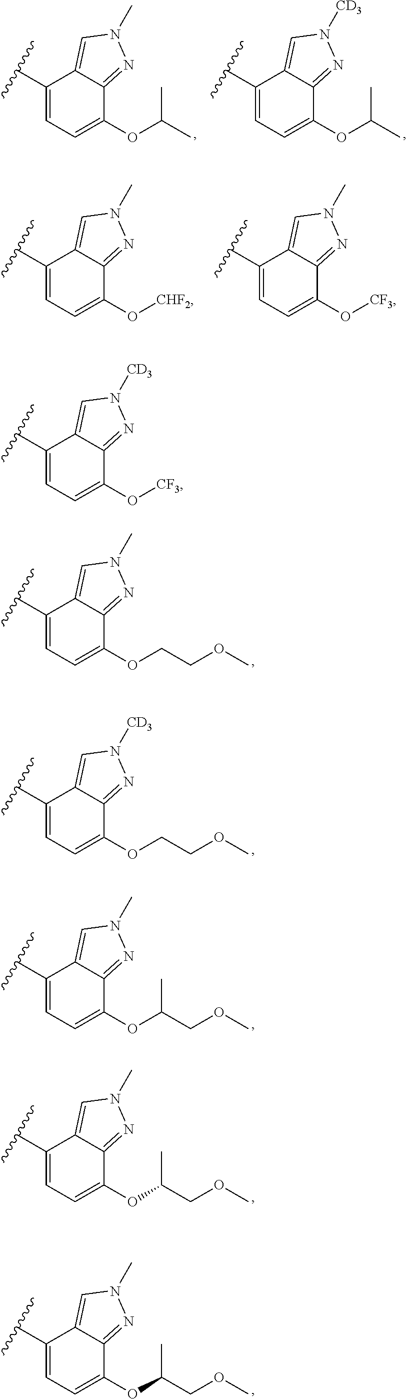

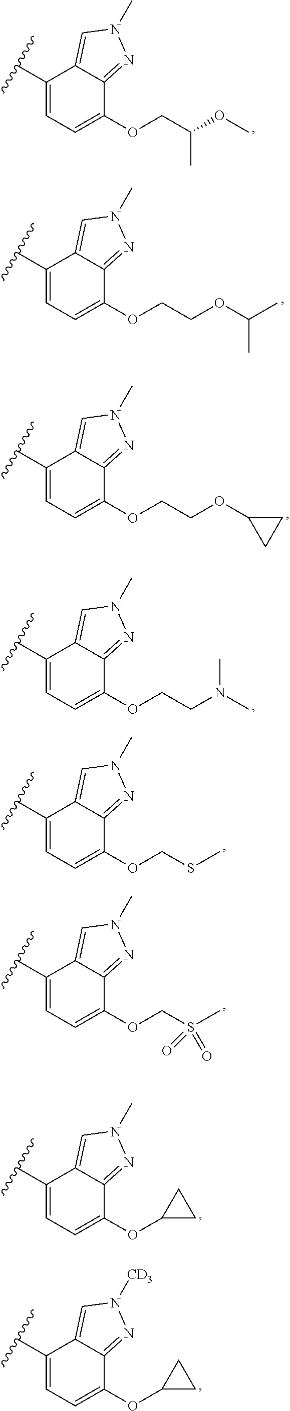

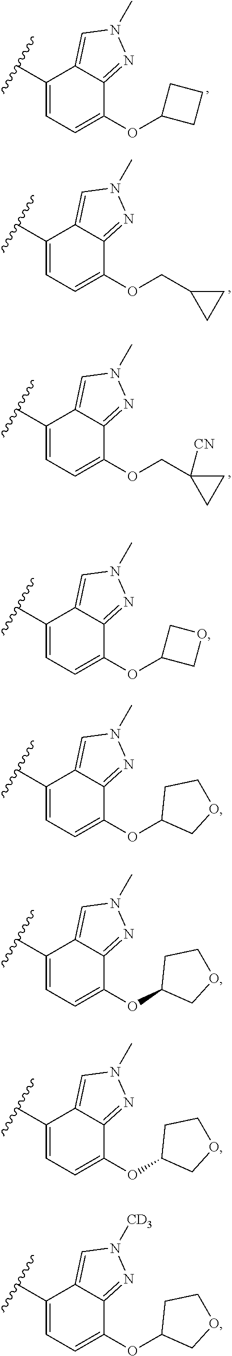

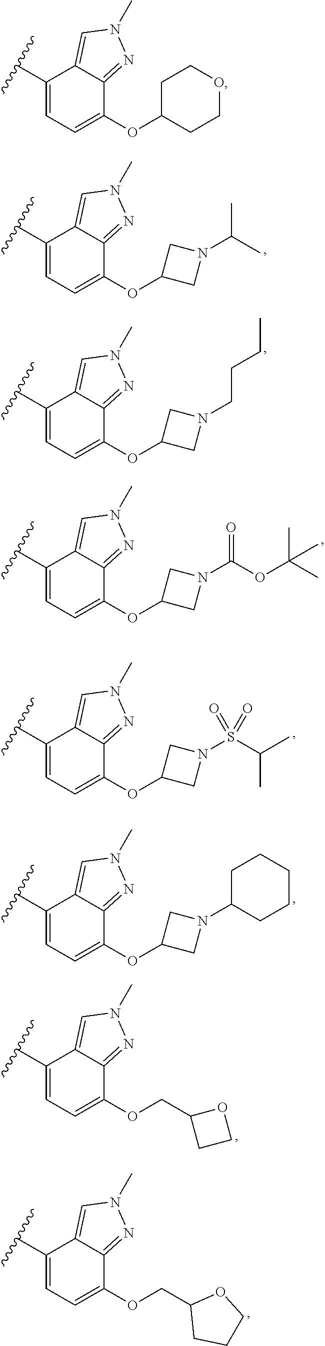

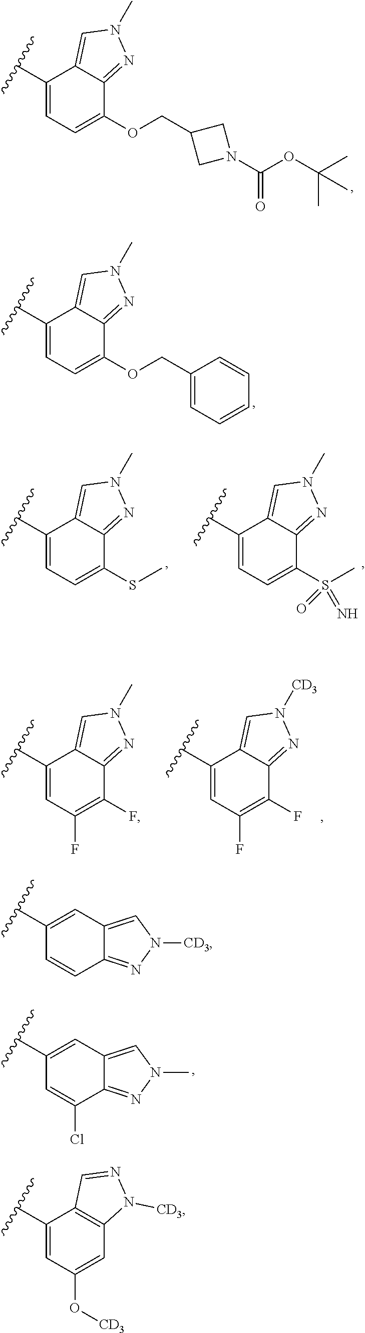

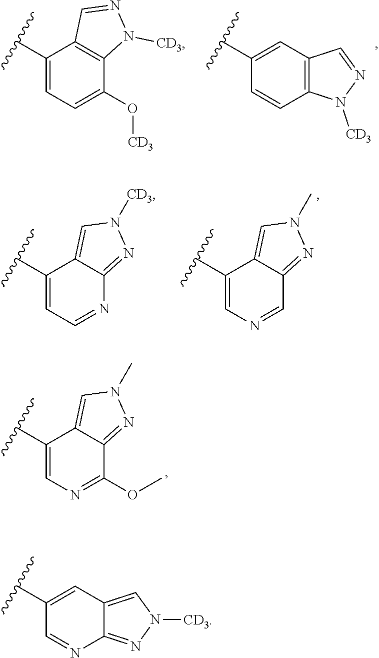 [SMILES: *c1ccc(OC([2H])([2H])[2H])c2c1cnn2C([2H])([2H])[2H] ; *c1ccc2c(cnn2C([2H])([2H])[2H])c1 ; *c1ccnc2nn(C([2H])([2H])[2H])cc12 ; *c1cncc2nn(C)cc12 ; *c1cnc(OC)c2nn(C)cc12 ; *c1cnc2nn(C([2H])([2H])[2H])cc2c1]

E16. The compound of any one of E1-E15.1, or a pharmaceutically acceptable salt thereof, wherein A¹ is selected from the group consisting of

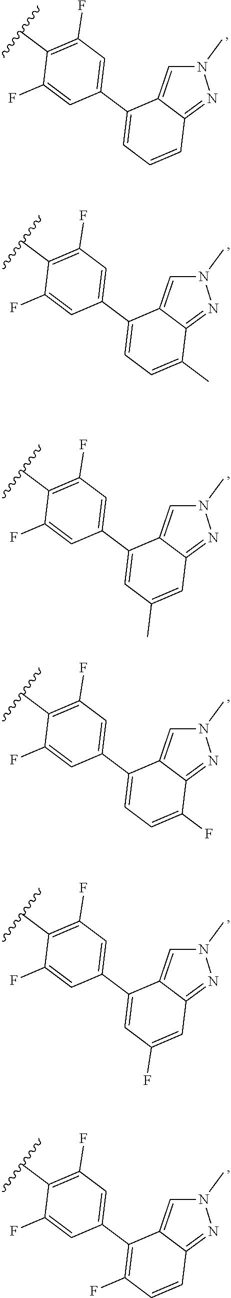 [SMILES: *c1c(F)cc(-c2cccc3nn(C)cc23)cc1F ; *c1c(F)cc(-c2ccc(C)c3nn(C)cc23)cc1F ; *c1c(F)cc(-c2cc(C)cc3nn(C)cc23)cc1F ; *c1c(F)cc(-c2ccc(F)c3nn(C)cc23)cc1F ; *c1c(F)cc(-c2cc(F)cc3nn(C)cc23)cc1F ; *c1c(F)cc(-c2c(F)ccc3nn(C)cc23)cc1F]

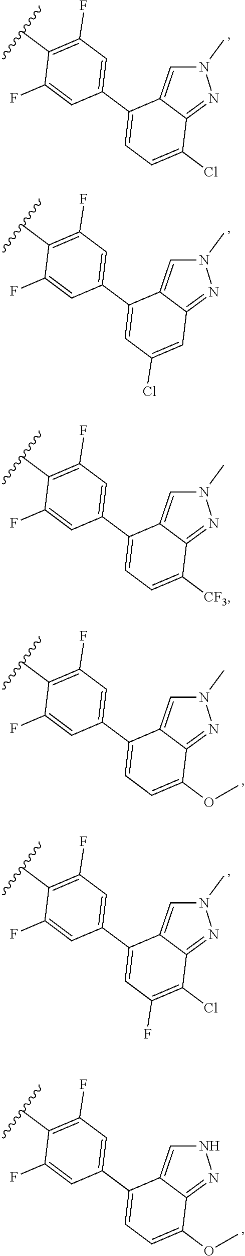 [SMILES: *c1c(F)cc(-c2ccc(Cl)c3nn(C)cc23)cc1F ; *c1c(F)cc(-c2cc(Cl)cc3nn(C)cc23)cc1F ; *c1c(F)cc(-c2ccc(C(F)(F)F)c3nn(C)cc23)cc1F ; *c1c(F)cc(-c2ccc(OC)c3nn(C)cc23)cc1F ; *c1c(F)cc(-c2cc(F)c(Cl)c3nn(C)cc23)cc1F ; *c1c(F)cc(-c2ccc(OC)c3n[nH]cc23)cc1F]

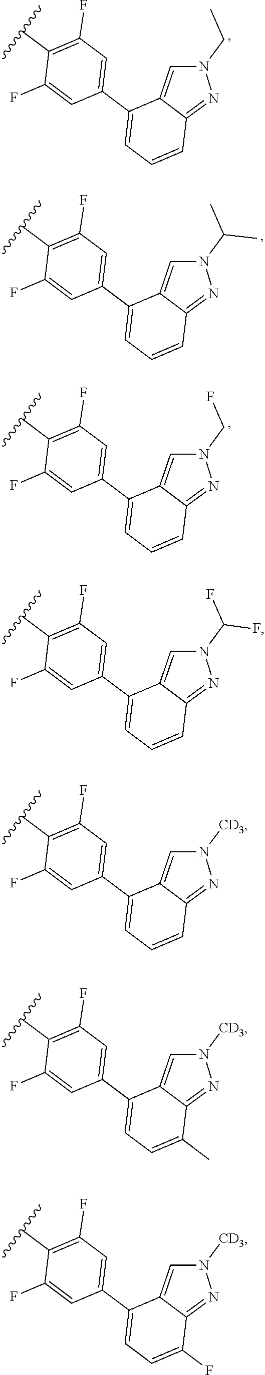

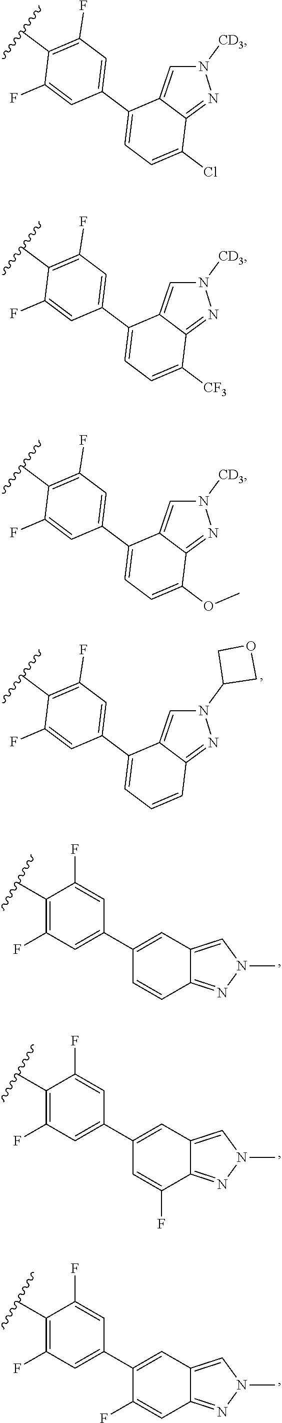

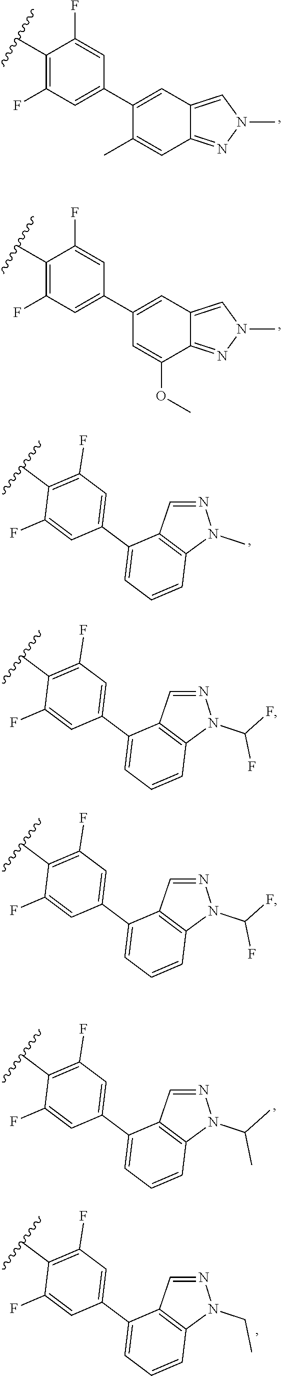

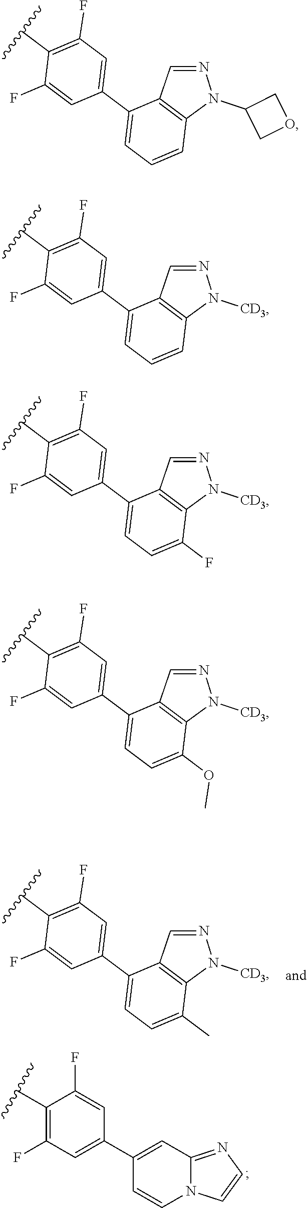 [SMILES: *c1c(F)cc(-c2cccc3c2cnn3C2COC2)cc1F ; *c1c(F)cc(-c2cccc3c2cnn3C([2H])([2H])[2H])cc1F ; *c1c(F)cc(-c2ccc(F)c3c2cnn3C([2H])([2H])[2H])cc1F ; *c1c(F)cc(-c2ccc(OC)c3c2cnn3C([2H])([2H])[2H])cc1F ; *c1c(F)cc(-c2ccc(C)c3c2cnn3C([2H])([2H])[2H])cc1F ; *c1c(F)cc(-c2ccn3ccnc3c2)cc1F]

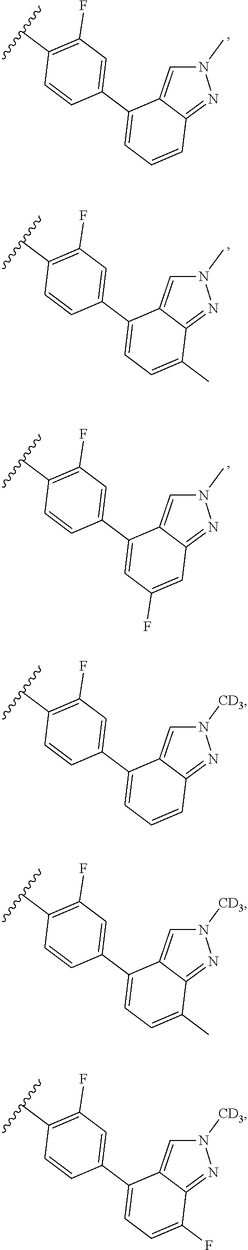 [SMILES: *c1ccc(-c2cccc3nn(C)cc23)cc1F ; *c1ccc(-c2ccc(C)c3nn(C)cc23)cc1F ; *c1ccc(-c2cc(F)cc3nn(C)cc23)cc1F ; *c1ccc(-c2cccc3nn(C([2H])([2H])[2H])cc23)cc1F ; *c1ccc(-c2ccc(C)c3nn(C([2H])([2H])[2H])cc23)cc1F ; *c1ccc(-c2ccc(F)c3nn(C([2H])([2H])[2H])cc23)cc1F]

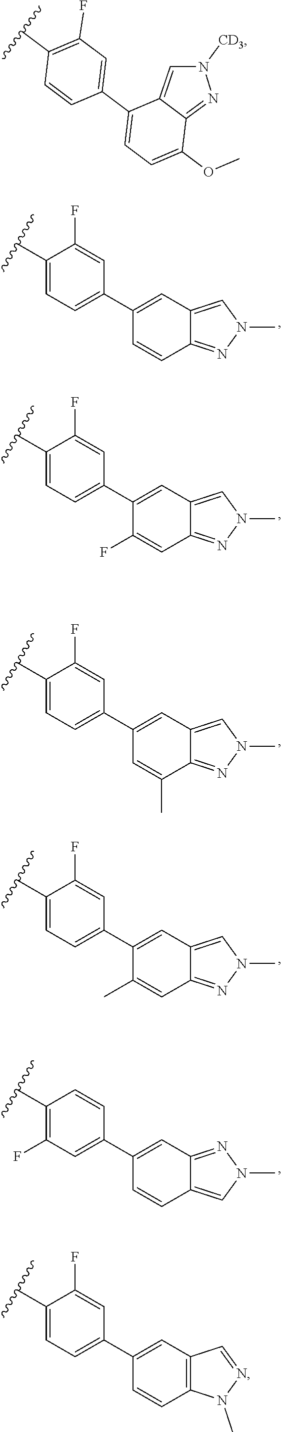

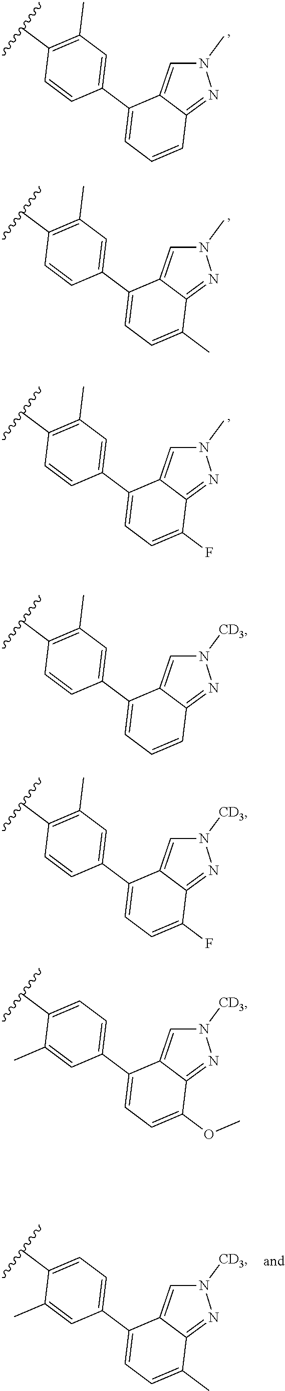

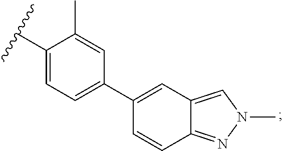 [SMILES: *c1ccc(-c2ccc3nn(C)cc3c2)cc1C]

or
A¹ is selected from the group consisting of

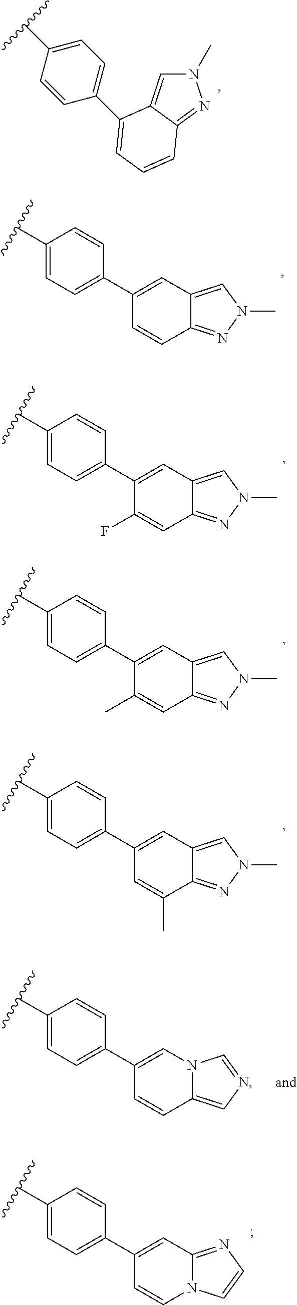

or
A¹ is

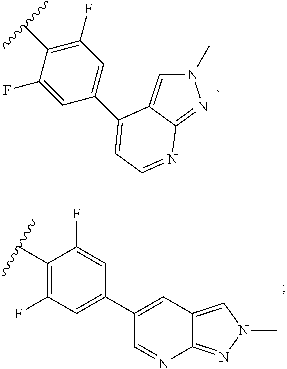 [SMILES: *c1c(F)cc(-c2ccnc3nn(C)cc23)cc1F ; *c1c(F)cc(-c2cnc3nn(C)cc3c2)cc1F]

or
A¹ is selected from the group consisting of

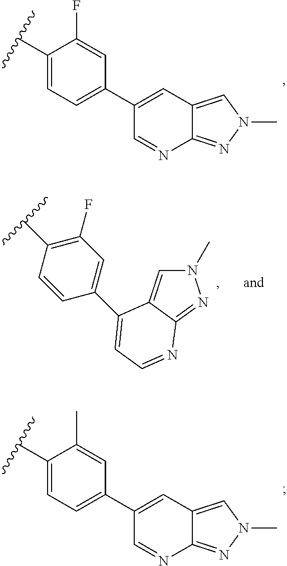 [SMILES: *c1ccc(-c2cnc3nn(C)cc3c2)cc1F ; *c1ccc(-c2ccnc3nn(C)cc23)cc1F ; *c1ccc(-c2cnc3nn(C)cc3c2)cc1C]

or
A¹ is

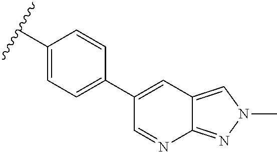 [SMILES: *c1ccc(-c2cnc3nn(C)cc3c2)cc1]

E17. The compound of any one of E1-E16, or a pharmaceutically acceptable salt thereof, wherein A¹ is selected from the group consisting of

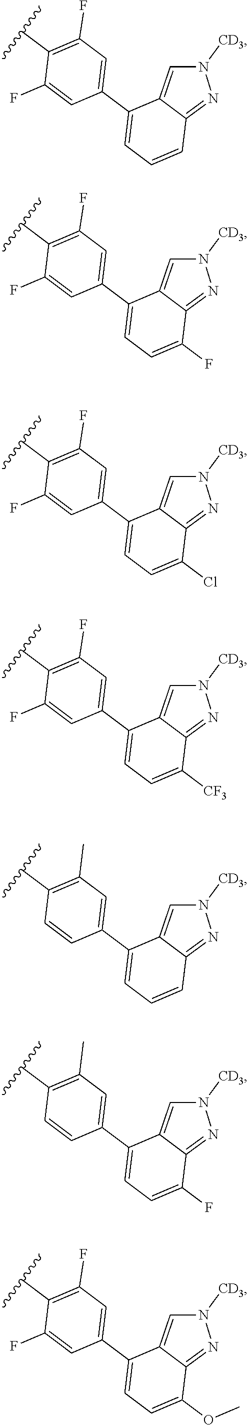

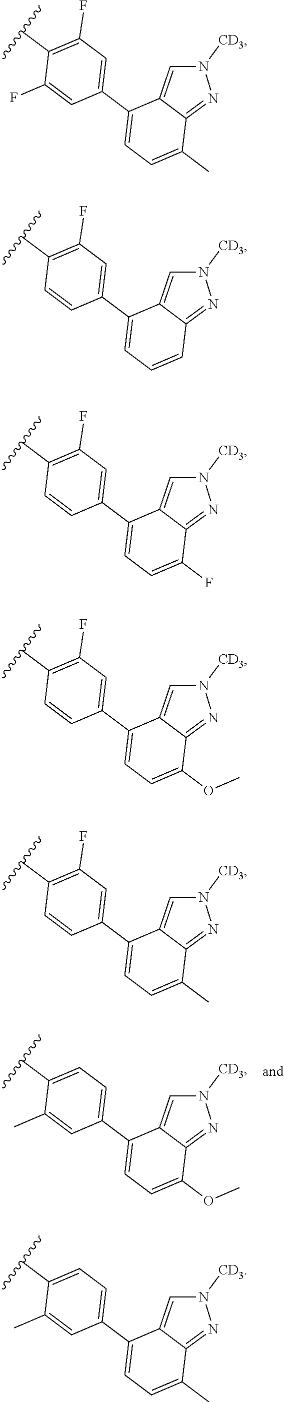

E18. The compound of any one of E1-E17, or a pharmaceutically acceptable salt thereof, wherein A¹ is

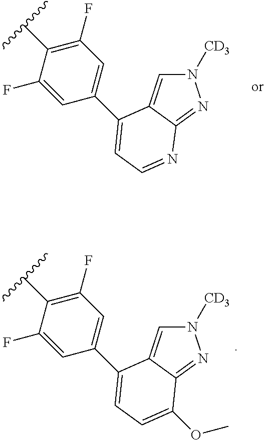 [SMILES: *c1c(F)cc(-c2ccnc3nn(C([2H])([2H])[2H])cc23)cc1F ; *c1c(F)cc(-c2ccc(OC)c3nn(C([2H])([2H])[2H])cc23)cc1F]

E19. The compound of any one of E1-E16, or a pharmaceutically acceptable salt thereof, wherein A¹ is selected from the group consisting of

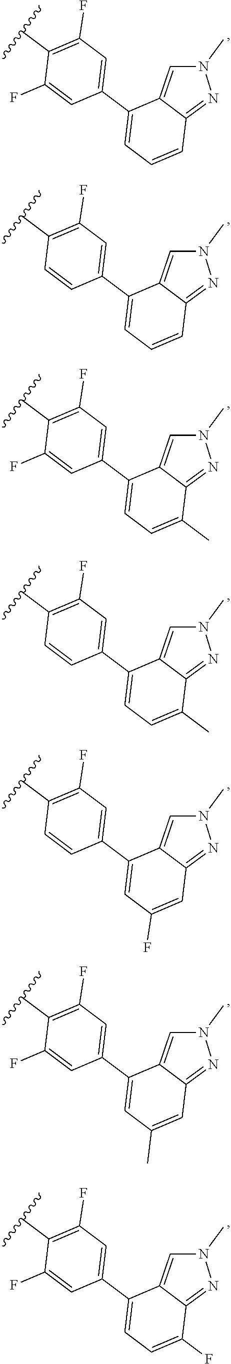

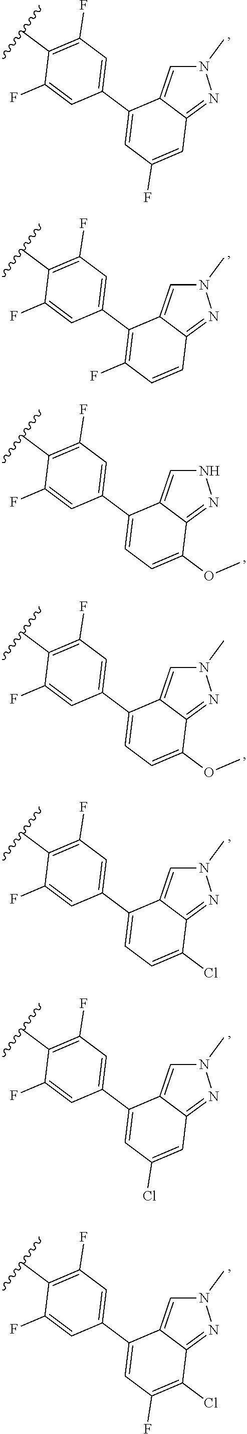

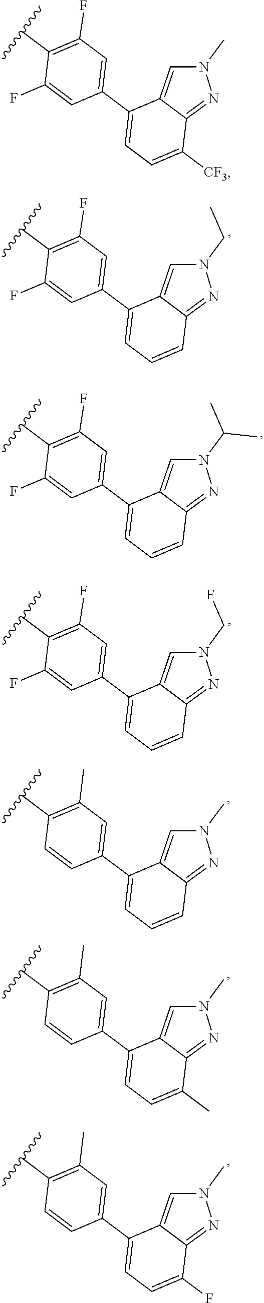

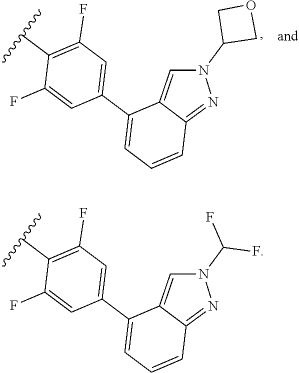 [SMILES: *c1c(F)cc(-c2cccc3nn(C4COC4)cc23)cc1F ; *c1c(F)cc(-c2cccc3nn(C(F)F)cc23)cc1F]

E20. The compound of any of E1-E19, or a pharmaceutically acceptable salt thereof, wherein A¹ is selected from the group consisting of

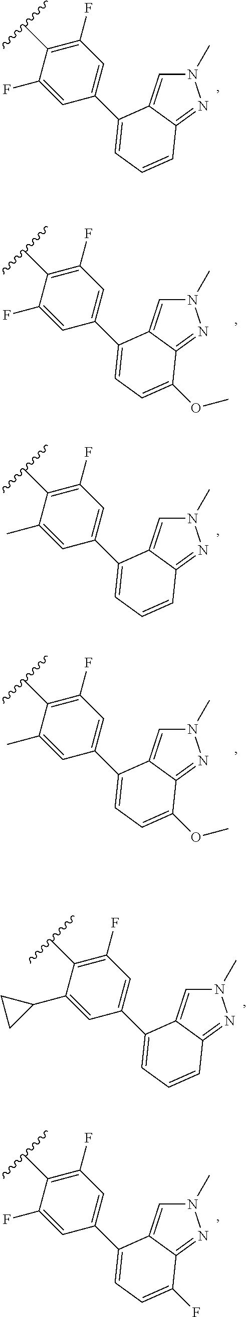

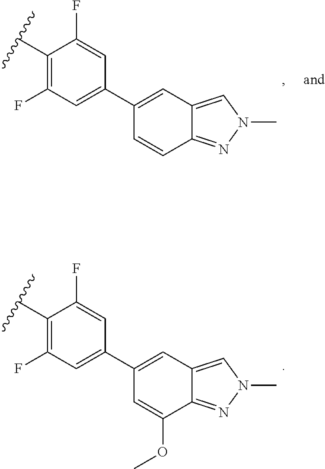 [SMILES: *c1c(F)cc(-c2ccc3nn(C)cc3c2)cc1F ; *c1c(F)cc(-c2cc(OC)c3nn(C)cc3c2)cc1F]

E20.1. The compound of E20, or a pharmaceutically acceptable salt thereof, wherein A¹ is

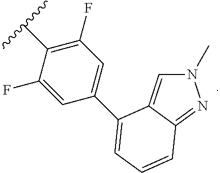 [SMILES: *c1c(F)cc(-c2cccc3nn(C)cc23)cc1F]

E20.2. The compound of E20.1, or a pharmaceutically acceptable salt thereof, wherein A¹ is

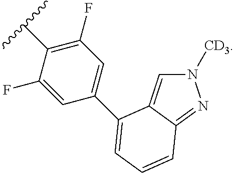 [SMILES: *c1c(F)cc(-c2cccc3nn(C([2H])([2H])[2H])cc23)cc1F]

E20.3. The compound of E20, or a pharmaceutically acceptable salt thereof, wherein A¹ is

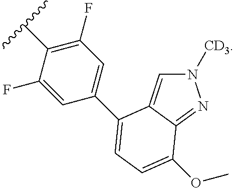 [SMILES: *c1c(F)cc(-c2ccc(OC)c3nn(C([2H])([2H])[2H])cc23)cc1F]

E20.4. The compound of E20.3, or a pharmaceutically acceptable salt thereof, wherein A¹ is

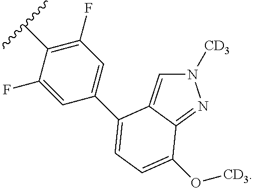 [SMILES: *c1c(F)cc(-c2ccc(OC([2H])([2H])[2H])c3nn(C([2H])([2H])[2H])cc23)cc1F]

E20.5. The compound of E20, or a pharmaceutically acceptable salt thereof, wherein A¹ is

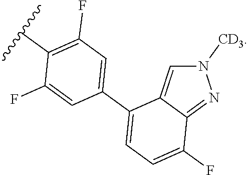 [SMILES: *c1c(F)cc(-c2ccc(F)c3nn(C([2H])([2H])[2H])cc23)cc1F]

E20.6. The compound of E20, or a pharmaceutically acceptable salt thereof, wherein A¹ is

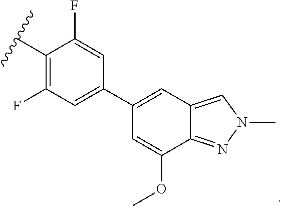 [SMILES: *c1c(F)cc(-c2cc(OC)c3nn(C)cc3c2)cc1F]

E20.7. The compound of E20, or a pharmaceutically acceptable salt thereof, wherein A¹ is

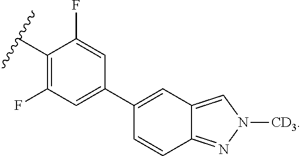 [SMILES: *c1c(F)cc(-c2ccc3nn(C([2H])([2H])[2H])cc3c2)cc1F]

E20.8. The compound of E20, or a pharmaceutically acceptable salt thereof, wherein A¹ is

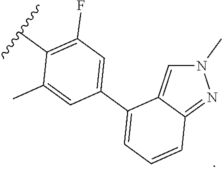

E20.9. The compound of E20.8, or a pharmaceutically acceptable salt thereof, wherein A¹ is

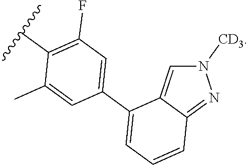 [SMILES: *c1c(C)cc(-c2cccc3nn(C([2H])([2H])[2H])cc23)cc1F]

E20.10. The compound of E20, or a pharmaceutically acceptable salt thereof, wherein A¹ is

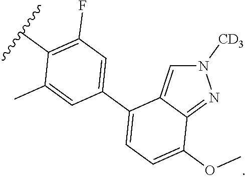

E20.11. The compound of E20, or a pharmaceutically acceptable salt thereof, wherein A¹ is

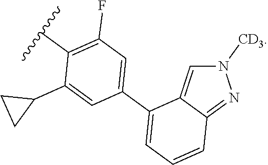 [SMILES: *c1c(F)cc(-c2cccc3nn(C([2H])([2H])[2H])cc23)cc1C1CC1]

E21. The compound of E20, or a pharmaceutically acceptable salt thereof, wherein A¹ is

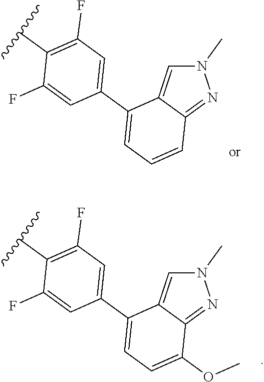 [SMILES: *c1c(F)cc(-c2cccc3nn(C)cc23)cc1F ; *c1c(F)cc(-c2ccc(OC)c3nn(C)cc23)cc1F]

E22. The compound of any of E1-E4.4, or a pharmaceutically acceptable salt thereof, wherein the ring system of R³ is a partially unsaturated 9-membered fused bicyclic heterocyclyl containing 1-3 nitrogen atoms, the heterocyclyl being unsubstituted or substituted as defined for R³ in any of E1-E1.3.

E22.1. The compound of E22, or a pharmaceutically acceptable salt thereof, wherein R³ is 7-oxo-6,7-dihydro-2H-pyrazolo[3,4-c]pyridin-4-yl optionally substituted with 1-2 substituents independently selected from the group consisting of C₁₋₄alkyl and C₁₋₄deuterioalkyl.

E22.2. The compound of E22.1, or a pharmaceutically acceptable salt thereof, wherein R³ is selected from the group consisting of

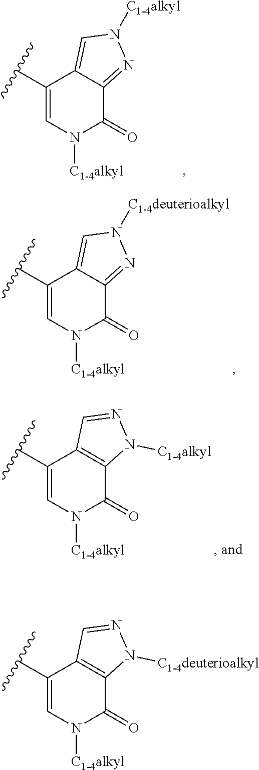

E22.3. The compound of E22.2, or a pharmaceutically acceptable salt thereof, wherein R³ is selected from the group consisting of

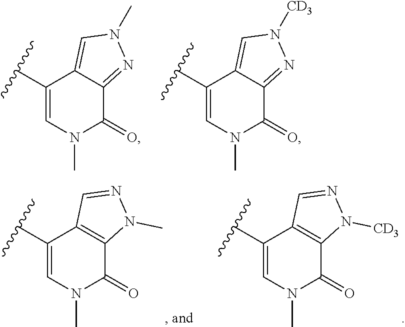

E23. The compound of any of E1-E22.3, or a pharmaceutically acceptable salt thereof, wherein Y¹ is H.

E24. The compound of any of E1-E22.3, or a pharmaceutically acceptable salt thereof, wherein Y¹ is halogen.

E24.1. The compound of E24, or a pharmaceutically acceptable salt thereof, wherein Y¹ is bromo.

E25. The compound of any of E1-E22.2, or a pharmaceutically acceptable salt thereof, wherein Y¹ is OH.

E26. The compound of any of E1-E22.2, or a pharmaceutically acceptable salt thereof, wherein Y¹ is OCD₃.

E27. The compound of any of E1-E26, or a pharmaceutically acceptable salt thereof, wherein Z¹ is N.

E28. The compound of any of E1-E26, or a pharmaceutically acceptable salt thereof, wherein Z¹ is CH.

E29. The compound of any of E1-E28, or a pharmaceutically acceptable salt thereof, wherein at least one of R³ and Y¹ comprises a deuterium label.

E29.1. The compound of E29, or a pharmaceutically acceptable salt thereof, wherein at least one of R³ and Y¹ comprises a CD₃ group.

E30. The compound of E1 or E1.2, selected from a compound of Table 19, or a pharmaceutically acceptable salt thereof.

E30.1. The compound of E30 selected from the group consisting of 6-(2,6-difluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.2. The compound of E30 selected from the group consisting of 6-(2,6-difluoro-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.3. The compound of E30 selected from the group consisting of 6-(2,6-difluoro-4-(2-methyl-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.4. The compound of E30 selected from the group consisting of 6-(2,6-difluoro-4-(6-fluoro-2-methyl-2H-indazol-5-yl)benzyl)-6,7-dihydro-5H-pyrrolo[3,4-b]pyridin-5-one-7,7-d₂, or a pharmaceutically acceptable salt thereof.

E30.5. The compound of E30 selected from the group consisting of 6-(2,6-difluoro-4-(imidazo[1,2-a]pyridin-7-yl)benzyl)-6,7-dihydro-5H-pyrrolo[3,4-b]pyridin-5-one-7,7-d₂, or a pharmaceutically acceptable salt thereof.

E30.6. The compound of E30 selected from the group consisting of 6-(4-(2,6-dimethyl-2H-indazol-5-yl)-2,6-difluorobenzyl)-6,7-dihydro-5H-pyrrolo[3,4-b]pyridin-5-one-7,7-d₂, or a pharmaceutically acceptable salt thereof.

E30.7. The compound of E30 selected from the group consisting of 6-(2,6-difluoro-4-(7-fluoro-2-methyl-2H-indazol-5-yl)benzyl)-6,7-dihydro-5H-pyrrolo[3,4-b]pyridin-5-one-7,7-d₂, or a pharmaceutically acceptable salt thereof.

E30.8. The compound of E30 selected from the group consisting of 6-(2,6-difluoro-4-(2-methyl-2H-indazol-5-yl)benzyl)-6,7-dihydro-5H-pyrrolo[3,4-b]pyridin-5-one-7,7-d², or a pharmaceutically acceptable salt thereof.

E30.9. The compound of E30 selected from the group consisting of 6-(2-fluoro-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.10. The compound of E30 selected from the group consisting of 6-(2-fluoro-4-(1-methyl-1H-indazol-5-yl)benzyl)-6,7-dihydro-5H-pyrrolo[3,4-b]pyridin-5-one-7,7-d₂, or a pharmaceutically acceptable salt thereof.

E30.11. The compound of E30 selected from the group consisting of 6-(4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.12. The compound of E30 selected from the group consisting of 6-(4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.13. The compound of E30 selected from the group consisting of 6-(4-(2-methyl-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.14. The compound of E30 selected from the group consisting of 6-(4-(2-methyl-2H-indazol-6-yl)benzyl)-6,7-dihydro-5H-pyrrolo[3,4-b]pyridin-5-one-7,7-d₂, or a pharmaceutically acceptable salt thereof.

E30.15. The compound of E30 selected from the group consisting of 6-(4-(6-fluoro-2-methyl-2H-indazol-5-yl)benzyl)-6,7-dihydro-5H-pyrrolo[3,4-b]pyridin-5-one-7,7-d₂, or a pharmaceutically acceptable salt thereof.

E30.16. The compound of E30 selected from the group consisting of 6-(4-(2,6-dimethyl-2H-indazol-5-yl)benzyl)-6,7-dihydro-5H-pyrrolo[3,4-b]pyridin-5-one-7,7-d₂, or a pharmaceutically acceptable salt thereof.

E30.17. The compound of E30 selected from the group consisting of 6-(4-(2,7-dimethyl-2H-indazol-5-yl)benzyl)-6,7-dihydro-5H-pyrrolo[3,4-b]pyridin-5-one-7,7-d₂, or a pharmaceutically acceptable salt thereof.

E30.18. The compound of E30 selected from the group consisting of 6-(4-(imidazo[1,5-a]pyridin-6-yl)benzyl)-6,7-dihydro-5H-pyrrolo[3,4-b]pyridin-5-one-7,7-d₂, or a pharmaceutically acceptable salt thereof.

E30.19. The compound of E30 selected from the group consisting of 6-(4-(imidazo[1,2-a]pyridin-7-yl)benzyl)-6,7-dihydro-5H-pyrrolo[3,4-b]pyridin-5-one-7,7-d₂, or a pharmaceutically acceptable salt thereof.

E30.20. The compound of E30 selected from the group consisting of 6-(2-fluoro-4-(2-methyl-2H-indazol-6-yl)benzyl)-6,7-dihydro-5H-pyrrolo[3,4-b]pyridin-5-one-7,7-d₂, or a pharmaceutically acceptable salt thereof.

E30.21. The compound of E30 selected from the group consisting of 6-(2-fluoro-4-(2-methyl-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.22. The compound of E30 selected from the group consisting of 6-(2-fluoro-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.23. The compound of E30 selected from the group consisting of 6-(2-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.24. The compound of E30 selected from the group consisting of 6-(2-fluoro-4-(6-fluoro-2-methyl-2H-indazol-5-yl)benzyl)-6,7-dihydro-5H-pyrrolo[3,4-b]pyridin-5-one-7,7-d₂, or a pharmaceutically acceptable salt thereof.

E30.25. The compound of E30 selected from the group consisting of 6-(4-(2,7-dimethyl-2H-indazol-5-yl)-2-fluorobenzyl)-6,7-dihydro-5H-pyrrolo[3,4-b]pyridin-5-one-7,7-d₂, or a pharmaceutically acceptable salt thereof.

E30.26. The compound of E30 selected from the group consisting of 6-(4-(2,6-dimethyl-2H-indazol-5-yl)-2-fluorobenzyl)-6,7-dihydro-5H-pyrrolo[3,4-b]pyridin-5-one-7,7-d₂, or a pharmaceutically acceptable salt thereof.

E30.27. The compound of E30 selected from the group consisting of 6-(4-(2,7-dimethyl-2H-indazol-4-yl)-2,6-difluorobenzyl)-6,7-dihydro-5H-pyrrolo[3,4-b]pyridin-5-one-7,7-d², or a pharmaceutically acceptable salt thereof.

E30.28. The compound of E30 selected from the group consisting of 6-(4-(2,7-dimethyl-2H-indazol-4-yl)-2-fluorobenzyl)-6,7-dihydro-5H-pyrrolo[3,4-b]pyridin-5-one-7,7-d₂, or a pharmaceutically acceptable salt thereof.

E30.29. The compound of E30 selected from the group consisting of 6-(2-methyl-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.30. The compound of E30 selected from the group consisting of 6-(4-(2,7-dimethyl-2H-indazol-4-yl)-2-methylbenzyl)-6,7-dihydro-5H-pyrrolo[3,4-b]pyridin-5-one-7,7-d₂, or a pharmaceutically acceptable salt thereof.

E30.31. The compound of E30 selected from the group consisting of 6-(2,6-difluoro-4-(2-(methyl-d³)-2H-indazol-4-yl)benzyl)-6,7-dihydro-5H-pyrrolo[3,4-b]pyridin-5-one-7,7-d₂, or a pharmaceutically acceptable salt thereof.

E30.32. The compound of E30 selected from the group consisting of 6-(4-(2,6-dimethyl-2H-indazol-4-yl)-2,6-difluorobenzyl)-6,7-dihydro-5H-pyrrolo[3,4-b]pyridin-5-one-7,7-d², or a pharmaceutically acceptable salt thereof.

E30.33. The compound of E30 selected from the group consisting of 6-(2-methyl-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.34. The compound of E30 selected from the group consisting of 6-(2-methyl-4-(2-methyl-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.35. The compound of E30 selected from the group consisting of 6-(2,6-difluoro-4-(1-methyl-1H-indazol-4-yl)benzyl)-6,7-dihydro-5H-pyrrolo[3,4-b]pyridin-5-one-7,7-d², or a pharmaceutically acceptable salt thereof.

E30.36. The compound of E30 selected from the group consisting of 6-(2,6-difluoro-4-(7-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.37. The compound of E30 selected from the group consisting of 6-(2,6-difluoro-4-(5-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.38. The compound of E30 selected from the group consisting of 6-(2,6-difluoro-4-(7-methoxy-2H-indazol-4-yl)benzyl)-6,7-dihydro-5H-pyrrolo[3,4-b]pyridin-5-one-7,7-d₂, or a pharmaceutically acceptable salt thereof.

E30.39. The compound of E30 selected from the group consisting of 6-(2,6-difluoro-4-(7-methoxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.40. The compound of E30 selected from the group consisting of 6-(4-(7-chloro-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.41. The compound of E30 selected from the group consisting of 6-(2,6-difluoro-4-(2-methyl-7-(trifluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.42. The compound of E30 selected from the group consisting of 6-(4-(2-ethyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.43. The compound of E30 selected from the group consisting of 6-(2,6-difluoro-4-(2-isopropyl-2H-indazol-4-yl)benzyl)-6,7-dihydro-5H-pyrrolo[3,4-b]pyridin-5-one-7,7-d₂, or a pharmaceutically acceptable salt thereof.

E30.44. The compound of E30 selected from the group consisting of 6-(2,6-difluoro-4-(2-(fluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.45. The compound of E30 selected from the group consisting of 6-(2,6-difluoro-4-(7-fluoro-2-(methyl-d³)-2H-indazol-4-yl)benzyl)-6,7-dihydro-5H-pyrrolo[3,4-b]pyridin-5-one-7,7-d₂, or a pharmaceutically acceptable salt thereof.

E30.46. The compound of E30 selected from the group consisting of 6-(2-methyl-4-(2-(methyl-d³)-2H-indazol-4-yl)benzyl)-6,7-dihydro-5H-pyrrolo[3,4-b]pyridin-5-one-7,7-d₂, or a pharmaceutically acceptable salt thereof.

E30.47. The compound of E30 selected from the group consisting of 6-(4-(7-fluoro-2-(methyl-d₃)-2H-indazol-4-yl)-2-methylbenzyl)-6,7-dihydro-5H-pyrrolo[3,4-b]pyridin-5-one-7,7-d₂, or a pharmaceutically acceptable salt thereof.

E30.48. The compound of E30 selected from the group consisting of 6-(4-(7-fluoro-2-methyl-2H-indazol-4-yl)-2-methylbenzyl)-6,7-dihydro-5H-pyrrolo[3,4-b]pyridin-5-one-7,7-d₂, or a pharmaceutically acceptable salt thereof.

E30.49. The compound of E30 selected from the group consisting of 6-(2,6-difluoro-4-(2-(oxetan-3-yl)-2H-indazol-4-yl)benzyl)-6,7-dihydro-5H-pyrrolo[3,4-b]pyridin-5-one-7,7-d₂, or a pharmaceutically acceptable salt thereof.

E30.50. The compound of E30 selected from the group consisting of 6-(4-(2-(difluoromethyl)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.51. The compound of E30 selected from the group consisting of 6-(2,6-difluoro-4-(7-methoxy-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.52. The compound of E30 selected from the group consisting of 6-(2,6-difluoro-4-(7-methyl-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.53. The compound of E30 selected from the group consisting of 6-(2-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.54. The compound of E30 selected from the group consisting of 6-(2-fluoro-4-(7-fluoro-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.55. The compound of E30 selected from the group consisting of 6-(2-fluoro-4-(7-methoxy-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.56. The compound of E30 selected from the group consisting of 6-(2-fluoro-4-(7-methyl-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.57. The compound of E30 selected from the group consisting of 6-(4-(7-methoxy-2-(methyl-d₃)-2H-indazol-4-yl)-2-methylbenzyl)-6,7-dihydro-5H-pyrrolo[3,4-b]pyridin-5-one-7,7-d₂, or a pharmaceutically acceptable salt thereof.

E30.58. The compound of E30 selected from the group consisting of 6-(2-methyl-4-(7-methyl-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.59. The compound of E30 selected from the group consisting of 6-(2,6-difluoro-4-(6-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.60. The compound of E30 selected from the group consisting of 6-(4-(1-(difluoromethyl)-1H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.61. The compound of E30 selected from the group consisting of 3-bromo-6-(2,6-difluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.62. The compound of E30 selected from the group consisting of 6-(2,6-difluoro-4-(2-methyl-2H-indazol-4-yl)benzyl)-3-hydroxy-6,7-dihydro-5H-pyrrolo[3,4-b]pyridin-5-one-7,7-d₂, or a pharmaceutically acceptable salt thereof.

E30.63. The compound of E30 selected from the group consisting of 6-(2,6-difluoro-4-(2-methyl-2H-indazol-4-yl)benzyl)-3-(methoxy-d₃)-6,7-dihydro-5H-pyrrolo[3,4-b]pyridin-5-one-7,7-d₂, or a pharmaceutically acceptable salt thereof.

E30.64. The compound of E30 selected from the group consisting of 6-(2,6-difluoro-4-(1-isopropyl-1H-indazol-4-yl)benzyl)-6,7-dihydro-5H-pyrrolo[3,4-b]pyridin-5-one-7,7-d₂, or a pharmaceutically acceptable salt thereof.

E30.65. The compound of E30 selected from the group consisting of 6-(4-(1-ethyl-1H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.66. The compound of E30 selected from the group consisting of 6-(2,6-difluoro-4-(1-(oxetan-3-yl)-1H-indazol-4-yl)benzyl)-6,7-dihydro-5H-pyrrolo[3,4-b]pyridin-5-one-7,7-d₂, or a pharmaceutically acceptable salt thereof.

E30.67. The compound of E30 selected from the group consisting of 6-(2,6-difluoro-4-(1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.68. The compound of E30 selected from the group consisting of 6-(2,6-difluoro-4-(7-fluoro-1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.69. The compound of E30 selected from the group consisting of 6-(2,6-difluoro-4-(7-methoxy-1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.70. The compound of E30 selected from the group consisting of 6-(2,6-difluoro-4-(7-methyl-1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.71. The compound of E30 selected from the group consisting of 6-(2-fluoro-4-(6-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.72. The compound of E30 selected from the group consisting of 6-(4-(6-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.73. The compound of E30 selected from the group consisting of 6-(4-(7-chloro-2-(methyl-d₃)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.74. The compound of E30 selected from the group consisting of 6-(2,6-difluoro-4-(2-(methyl-d₃)-7-(trifluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.75. The compound of E30 selected from the group consisting of 6-(4-(6-chloro-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.76. The compound of E30 selected from the group consisting of 6-(2,6-difluoro-4-(7-methoxy-2-methyl-2H-indazol-5-yl)benzyl)-6,7-dihydro-5H-pyrrolo[3,4-b]pyridin-5-one-7,7-d₂, or a pharmaceutically acceptable salt thereof.

E30.77. The compound of E30 selected from the group consisting of 6-(4-(7-chloro-6-fluoro-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.78. The compound of E30 selected from the group consisting of 6-(2,6-difluoro-4-(2-(methyl-d₃)-2H-indazol-4-yl)benzyl)-3-(methoxy-d₃)-6,7-dihydro-5H-pyrrolo[3,4-b]pyridin-5-one-7,7-d₂, or a pharmaceutically acceptable salt thereof.

E30.79. The compound of E30 selected from the group consisting of 4-(3,5-difluoro-4-((5-oxo-5,7-dihydro-6H-pyrrolo[3,4-b]pyridin-6-yl-7,7-d₂)methyl)phenyl)-2,6-dimethyl-2,6-dihydro-7H-pyrazolo[3,4-c]pyridin-7-one, or a pharmaceutically acceptable salt thereof.

E30.80. The compound of E30 selected from the group consisting of 4-(3,5-difluoro-4-((5-oxo-5,7-dihydro-6H-pyrrolo[3,4-b]pyridin-6-yl-7,7-d₂)methyl)phenyl)-1,6-dimethyl-1,6-dihydro-7H-pyrazolo[3,4-c]pyridin-7-one, or a pharmaceutically acceptable salt thereof.

E30.81. The compound of E30 selected from the group consisting of 6-(4-(6-(difluoromethyl)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.82. The compound of E30 selected from the group consisting of 6-(2-fluoro-4-(2-methyl-2H-indazol-4-yl)benzyl)-3-(methoxy-d₃)-6,7-dihydro-5H-pyrrolo[3,4-b]pyridin-5-one-7,7-d₂, or a pharmaceutically acceptable salt thereof.

E30.83. The compound of E30 selected from the group consisting of 3-(methoxy-d₃)-6-(4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.84. The compound of E30 selected from the group consisting of 6-(4-(6-fluoro-2-methyl-2H-indazol-4-yl)-2-methylbenzyl)-6,7-dihydro-5H-pyrrolo[3,4-b]pyridin-5-one-7,7-d₂, or a pharmaceutically acceptable salt thereof.

E30.85. The compound of E30 selected from the group consisting of 6-(4-(6-chloro-2-(methyl-d₃)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.86. The compound of E30 selected from the group consisting of 6-(2,6-difluoro-4-(6-fluoro-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.87. The compound of E30 selected from the group consisting of 6-(2,6-difluoro-4-(6-(methoxy-d₃)-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.88. The compound of E30 selected from the group consisting of 6-(2,6-difluoro-4-(6-(methoxy-d₃)-1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.89. The compound of E30 selected from the group consisting of 6-(4-(7-chloro-2-methyl-2H-indazol-5-yl)-2,6-difluorobenzyl)-6,7-dihydro-5H-pyrrolo[3,4-b]pyridin-5-one-7,7-d₂, or a pharmaceutically acceptable salt thereof.

E30.90. The compound of E30 selected from the group consisting of 6-(2,6-difluoro-4-(6-methoxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.91. The compound of E30 selected from the group consisting of 4-(3,5-difluoro-4-((5-oxo-5,7-dihydro-6H-pyrrolo[3,4-b]pyridin-6-yl-7,7-d₂)methyl)phenyl)-6-methyl-2-(methyl-d₃)-2,6-dihydro-7H-pyrazolo[3,4-c]pyridin-7-one, or a pharmaceutically acceptable salt thereof.

E30.92. The compound of E30 selected from the group consisting of 4-(3,5-difluoro-4-((5-oxo-5,7-dihydro-6H-pyrrolo[3,4-b]pyridin-6-yl-7,7-d₂)methyl)phenyl)-6-methyl-1-(methyl-d₃)-1,6-dihydro-7H-pyrazolo[3,4-c]pyridin-7-one, or a pharmaceutically acceptable salt thereof.

E30.93. The compound of E30 selected from the group consisting of 6-(2,6-difluoro-4-(7-(methoxy-d₃)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.94. The compound of E30 selected from the group consisting of 6-(2,6-difluoro-4-(7-(methoxy-d₃)-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.95. The compound of E30 selected from the group consisting of 6-(2,6-difluoro-4-(7-(methoxy-d₃)-1-(methyl-d₃)-1H-indazol-4-yl)benzyl)-6,7-dihydro-5H-pyrrolo[3,4-b]pyridin-5-one-7,7-d₂, or a pharmaceutically acceptable salt thereof.

E30.96. The compound of E30 selected from the group consisting of 6-(4-(7-ethoxy-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.97. The compound of E30 selected from the group consisting of 6-(4-(7-ethoxy-2-(methyl-d₃)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.98. The compound of E30 selected from the group consisting of 6-(2,6-difluoro-4-(7-isopropoxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.99. The compound of E30 selected from the group consisting of 6-(2,6-difluoro-4-(7-isopropoxy-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.100. The compound of E30 selected from the group consisting of 6-(2,6-difluoro-4-(2-(methyl-d₃)-2H-indazol-5-yl)benzyl)-6,7-dihydro-5H-pyrrolo[3,4-b]pyridin-5-one-7,7-d₂, or a pharmaceutically acceptable salt thereof.

E30.101. The compound of E30 selected from the group consisting of 6-(2,6-difluoro-4-(1-(methyl-d₃)-1H-indazol-5-yl)benzyl)-6,7-dihydro-5H-pyrrolo[3,4-b]pyridin-5-one-7,7-d₂, or a pharmaceutically acceptable salt thereof.

E30.102. The compound of E30 selected from the group consisting of 6-(4-(7-(difluoromethoxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.103. The compound of E30 selected from the group consisting of 6-(2,6-difluoro-4-(2-methyl-7-(trifluoromethoxy)-2H-indazol-4-yl)benzyl)-6,7-dihydro-5H-pyrrolo[3,4-b]pyridin-5-one-7,7-d₂, or a pharmaceutically acceptable salt thereof.

E30.104. The compound of E30 selected from the group consisting of 6-(2,6-difluoro-4-(2-(methyl-d₃)-7-(trifluoromethoxy)-2H-indazol-4-yl)benzyl)-6,7-dihydro-5H-pyrrolo[3,4-b]pyridin-5-one-7,7-d₂, or a pharmaceutically acceptable salt thereof.

E30.105. The compound of E30 selected from the group consisting of 6-(4-(6,7-difluoro-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.106. The compound of E30 selected from the group consisting of 6-(4-(6,7-difluoro-2-(methyl-d₃)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.107. The compound of E30 selected from the group consisting of 6-(2-fluoro-4-(7-(methoxy-d₃)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.108. The compound of E30 selected from the group consisting of 6-(4-(7-ethoxy-2-methyl-2H-indazol-4-yl)-2-fluorobenzyl)-6,7-dihydro-5H-pyrrolo[3,4-b]pyridin-5-one-7,7-d₂, or a pharmaceutically acceptable salt thereof.

E30.109. The compound of E30 selected from the group consisting of 6-(2-fluoro-4-(7-isopropoxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.110. The compound of E30 selected from the group consisting of 6-(2-fluoro-4-(2-methyl-7-(trifluoromethoxy)-2H-indazol-4-yl)benzyl)-6,7-dihydro-5H-pyrrolo[3,4-b]pyridin-5-one-7,7-d₂, or a pharmaceutically acceptable salt thereof.

E30.111. The compound of E30 selected from the group consisting of 6-(2-fluoro-4-(2-methyl-2H-indazol-5-yl)benzyl)-3-(methoxy-d₃)-6,7-dihydro-5H-pyrrolo[3,4-b]pyridin-5-one-7,7-d₂, or a pharmaceutically acceptable salt thereof.

E30.112. The compound of E30 selected from the group consisting of 6-(2,6-difluoro-4-(2-methyl-2H-indazol-5-yl)benzyl)-3-(methoxy-d₃)-6,7-dihydro-5H-pyrrolo[3,4-b]pyridin-5-one-7,7-d₂, or a pharmaceutically acceptable salt thereof.

E30.113. The compound of E30 selected from the group consisting of 6-(2,6-difluoro-4-(7-(2-methoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.114. The compound of E30 selected from the group consisting of 6-(2,6-difluoro-4-(7-(2-methoxyethoxy)-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.115. The compound of E30 selected from the group consisting of 6-(2,6-difluoro-4-(2-(methyl-d₃)-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.116. The compound of E30 selected from the group consisting of 6-(2-fluoro-4-(7-(2-methoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.117. The compound of E30 selected from the group consisting of 6-(2-fluoro-4-(7-(2-methoxyethoxy)-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.118. The compound of E30 selected from the group consisting of 6-(2-fluoro-4-(2-(methyl-d₃)-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.119. The compound of E30 selected from the group consisting of 6-(2,6-difluoro-4-(2-methyl-2H-pyrazolo[3,4-c]pyridin-4-yl)benzyl)-6,7-dihydro-5H-pyrrolo[3,4-b]pyridin-5-one-7,7-d₂, or a pharmaceutically acceptable salt thereof.

E30.120. The compound of E30 selected from the group consisting of 6-(2-fluoro-4-(2-methyl-2H-pyrazolo[3,4-c]pyridin-4-yl)benzyl)-6,7-dihydro-5H-pyrrolo[3,4-b]pyridin-5-one-7,7-d₂, or a pharmaceutically acceptable salt thereof.

E30.121. The compound of E30 selected from the group consisting of 6-(2,6-difluoro-4-(7-methoxy-2-methyl-2H-pyrazolo[3,4-c]pyridin-4-yl)benzyl)-6,7-dihydro-5H-pyrrolo[3,4-b]pyridin-5-one-7,7-d₂, or a pharmaceutically acceptable salt thereof.

E30.122. The compound of E30 selected from the group consisting of 6-(4-(7-cyclopropyl-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.123. The compound of E30 selected from the group consisting of 6-(4-(7-(difluoromethyl)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.124. The compound of E30 selected from the group consisting of 6-(4-(7-cyclopropoxy-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.125. The compound of E30 selected from the group consisting of 6-(4-(7-cyclobutoxy-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.126. The compound of E30 selected from the group consisting of 6-(2,6-difluoro-4-(2-(methyl-d₃)-2H-indazol-4-yl)benzyl)-3-(2-(pyridin-2-yl)ethoxy)-6,7-dihydro-5H-pyrrolo[3,4-b]pyridin-5-one-7,7-d₂, or a pharmaceutically acceptable salt thereof.

E30.127. The compound of E30 selected from the group consisting of 6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.128. The compound of E30 selected from the group consisting of 6-(2,6-difluoro-4-(2-(methyl-d₃)-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.129. The compound of E30 selected from the group consisting of 6-(4-(7-(benzyloxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.130. The compound of E30 selected from the group consisting of 6-(2,6-difluoro-4-(7-hydroxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.131. The compound of E30 selected from the group consisting of 6-(4-(7-(2-(dimethylamino)ethoxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.132. The compound of E30 selected from the group consisting of 6-(2,6-difluoro-4-(7-hydroxy-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.133. The compound of E30 selected from the group consisting of 6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.134. The compound of E30 selected from the group consisting of 6-(2,6-difluoro-4-(2-methyl-7-((tetrahydrofuran-2-yl)methoxy)-2H-indazol-4-yl)benzyl)-6,7-dihydro-5H-pyrrolo[3,4-b]pyridin-5-one-7,7-d₂, or a pharmaceutically acceptable salt thereof.

E30.135. The compound of E30 selected from the group consisting of 6-(2,6-difluoro-4-(2-methyl-7-(oxetan-2-ylmethoxy)-2H-indazol-4-yl)benzyl)-6,7-dihydro-5H-pyrrolo[3,4-b]pyridin-5-one-7,7-d₂, or a pharmaceutically acceptable salt thereof.

E30.136. The compound of E30 selected from the group consisting of (S)-6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.137. The compound of E30 selected from the group consisting of (R)-6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.138. The compound of E30 selected from the group consisting of 6-(4-(7-(cyclopropylmethoxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.139. The compound of E30 selected from the group consisting of 6-(2,6-difluoro-4-(2-methyl-7-(oxetan-3-yloxy)-2H-indazol-4-yl)benzyl)-6,7-dihydro-5H-pyrrolo[3,4-b]pyridin-5-one-7,7-d₂, or a pharmaceutically acceptable salt thereof.

E30.140. The compound of E30 selected from the group consisting of 6-(2,6-difluoro-4-(2-methyl-7-((tetrahydro-2H-pyran-4-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.141. The compound of E30 selected from the group consisting of 6-(2,6-difluoro-4-(2-(methyl-d₃)-2H-indazol-4-yl)benzyl)-3-hydroxy-6,7-dihydro-5H-pyrrolo[3,4-b]pyridin-5-one-7,7-d₂, or a pharmaceutically acceptable salt thereof.

E30.142. The compound of E30 selected from the group consisting of 6-(2-fluoro-4-(7-methoxy-2-methyl-2H-indazol-4-yl)benzyl)-3-(methoxy-d₃)-6,7-dihydro-5H-pyrrolo[3,4-b]pyridin-5-one-7,7-d₂, or a pharmaceutically acceptable salt thereof.

E30.143. The compound of E30 selected from the group consisting of 6-(2-fluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.144. The compound of E30 selected from the group consisting of (S)-6-(2-fluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.145. The compound of E30 selected from the group consisting of 6-(2-fluoro-4-(2-methyl-7-(oxetan-2-ylmethoxy)-2H-indazol-4-yl)benzyl)-6,7-dihydro-5H-pyrrolo[3,4-b]pyridin-5-one-7,7-d₂, or a pharmaceutically acceptable salt thereof.

E30.146. The compound of E30 selected from the group consisting of 6-(2,6-difluoro-4-(7-methoxy-2-methyl-2H-indazol-4-yl)benzyl)-3-(methoxy-d₃)-6,7-dihydro-5H-pyrrolo[3,4-b]pyridin-5-one-7,7-d₂, or a pharmaceutically acceptable salt thereof.

E30.147. The compound of E30 selected from the group consisting of 6-(2-fluoro-4-(2-methyl-7-((tetrahydrofuran-2-yl)methoxy)-2H-indazol-4-yl)benzyl)-6,7-dihydro-5H-pyrrolo[3,4-b]pyridin-5-one-7,7-d₂, or a pharmaceutically acceptable salt thereof.

E30.148. The compound of E30 selected from the group consisting of (R)-6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.149. The compound of E30 selected from the group consisting of 6-(2,6-difluoro-4-(7-(2-isopropoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.150. The compound of E30 selected from the group consisting of 6-(4-(7-(2-cyclopropoxyethoxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.151. The compound of E30 selected from the group consisting of (R)-6-(2-fluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂, or a pharmaceutically acceptable salt thereof.

E30.152. The compound of E30 selected from the group consisting of (S)-6-(2-fluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.153. The compound of E30 selected from the group consisting of 6-(2-fluoro-4-(7-(2-isopropoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.154. The compound of E30 selected from the group consisting of 6-(4-(7-(2-cyclopropoxyethoxy)-2-methyl-2H-indazol-4-yl)-2-fluorobenzyl)-6,7-dihydro-5H-pyrrolo[3,4-b]pyridin-5-one-7,7-d₂, or a pharmaceutically acceptable salt thereof.

E30.155. The compound of E30 selected from the group consisting of 6-(2-fluoro-6-methyl-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.156. The compound of E30 selected from the group consisting of 6-(2-fluoro-6-methyl-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.157. The compound of E30 selected from the group consisting of 6-(2-fluoro-6-methyl-4-(7-methyl-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.158. The compound of E30 selected from the group consisting of 6-(2-fluoro-4-(7-fluoro-2-(methyl-d₃)-2H-indazol-4-yl)-6-methylbenzyl)-6,7-dihydro-5H-pyrrolo[3,4-b]pyridin-5-one-7,7-d₂, or a pharmaceutically acceptable salt thereof.

E30.159. The compound of E30 selected from the group consisting of 6-(2-fluoro-4-(7-methoxy-2-(methyl-d₃)-2H-indazol-4-yl)-6-methylbenzyl)-6,7-dihydro-5H-pyrrolo[3,4-b]pyridin-5-one-7,7-d₂, or a pharmaceutically acceptable salt thereof.

E30.160. The compound of E30 selected from the group consisting of 6-(2-fluoro-6-methyl-4-(2-(methyl-d₃)-2H-indazol-5-yl)benzyl)-6,7-dihydro-5H-pyrrolo[3,4-b]pyridin-5-one-7,7-d₂, or a pharmaceutically acceptable salt thereof.

E30.161. The compound of E30 selected from the group consisting of 6-(2-chloro-6-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.162. The compound of E30 selected from the group consisting of 6-(2-chloro-4-(2,7-dimethyl-2H-indazol-4-yl)-6-fluorobenzyl)-6,7-dihydro-5H-pyrrolo[3,4-b]pyridin-5-one-7,7-d₂, or a pharmaceutically acceptable salt thereof.

E30.163. The compound of E30 selected from the group consisting of 6-(2-chloro-6-fluoro-4-(7-methoxy-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.164. The compound of E30 selected from the group consisting of 6-(2-chloro-6-fluoro-4-(7-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.165. The compound of E30 selected from the group consisting of 6-(2-chloro-6-fluoro-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.166. The compound of E30 selected from the group consisting of 6-(2-chloro-6-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.167. The compound of E30 selected from the group consisting of 6-(2-chloro-6-fluoro-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.168. The compound of E30 selected from the group consisting of 6-(2-chloro-6-fluoro-4-(2-methyl-2H-pyrazolo[3,4-b]pyridin-5-yl)benzyl)-6,7-dihydro-5H-pyrrolo[3,4-b]pyridin-5-one-7,7-d₂, or a pharmaceutically acceptable salt thereof.

E30.169. The compound of E30 selected from the group consisting of 6-(2-chloro-4-(7-cyclopropoxy-2-methyl-2H-indazol-4-yl)-6-fluorobenzyl)-6,7-dihydro-5H-pyrrolo[3,4-b]pyridin-5-one-7,7-d₂, or a pharmaceutically acceptable salt thereof.

E30.170. The compound of E30 selected from the group consisting of 6-(2-chloro-6-fluoro-4-(7-(2-methoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.171. The compound of E30 selected from the group consisting of (S)-6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.172. The compound of E30 selected from the group consisting of (R)-6-(2,6-difluoro-4-(7-(2-methoxypropoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.173. The compound of E30 selected from the group consisting of tert-butyl 3-((4-(3,5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)azetidine-1-carboxylate, or a pharmaceutically acceptable salt thereof.

E30.174. The compound of E30 selected from the group consisting of 1-(((4-(3,5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)cyclopropane-1-carbonitrile, or a pharmaceutically acceptable salt thereof.

E30.175. The compound of E30 selected from the group consisting of (R)-6-(2-fluoro-4-(7-(2-methoxypropoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.176. The compound of E30 selected from the group consisting of tert-butyl 3-((4-(3-fluoro-4-((5-oxo-5,7-dihydro-6H-pyrrolo[3,4-b]pyridin-6-yl-7,7-d₂)methyl)phenyl)-2-methyl-2H-indazol-7-yl)oxy)azetidine-1-carboxylate, or a pharmaceutically acceptable salt thereof.

E30.177. The compound of E30 selected from the group consisting of 6-(2-fluoro-4-(7-(2-methoxyethoxy)-2-methyl-2H-indazol-4-yl)-6-methylbenzyl)-6,7-dihydro-5H-pyrrolo[3,4-b]pyridin-5-one-7,7-d₂, or a pharmaceutically acceptable salt thereof.

E30.178. The compound of E30 selected from the group consisting of 1-(((4-(3-fluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)cyclopropane-1-carbonitrile, or a pharmaceutically acceptable salt thereof.

E30.179. The compound of E30 selected from the group consisting of tert-butyl 3-(((4-(3, 5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)azetidine-1-carboxylate, or a pharmaceutically acceptable salt thereof.

E30.180. The compound of E30 selected from the group consisting of 6-(2,6-difluoro-4-(7-((1-isopropylazetidin-3-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.181. The compound of E30 selected from the group consisting of 6-(2,6-difluoro-4-(2-methyl-7-(methylthio)-2H-indazol-4-yl)benzyl)-6,7-dihydro-5H-pyrrolo[3,4-b]pyridin-5-one-7,7-d₂, or a pharmaceutically acceptable salt thereof.

E30.182. The compound of E30 selected from the group consisting of 6-(2,6-difluoro-4-(2-methyl-7-(S-methylsulfonimidoyl)-2H-indazol-4-yl)benzyl)-6,7-dihydro-5H-pyrrolo[3,4-b]pyridin-5-one-7,7-d₂, or a pharmaceutically acceptable salt thereof.

E30.183. The compound of E30 selected from the group consisting of 6-(4-(7-cyclopropoxy-2-(methyl-d₃)-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.184. The compound of E30 selected from the group consisting of 6-(4-(7-((1-butylazetidin-3-yl)oxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.185. The compound of E30 selected from the group consisting of 6-(2,6-difluoro-4-(2-methyl-7-((methylthio)methoxy)-2H-indazol-4-yl)benzyl)-6,7-dihydro-5H-pyrrolo[3,4-b]pyridin-5-one-7,7-d₂, or a pharmaceutically acceptable salt thereof.

E30.186. The compound of E30 selected from the group consisting of 6-(2,6-difluoro-4-(2-methyl-7-((methylsulfonyl)methoxy)-2H-indazol-4-yl)benzyl)-6,7-dihydro-5H-pyrrolo[3,4-b]pyridin-5-one-7,7-d₂, or a pharmaceutically acceptable salt thereof.

E30.187. The compound of E30 selected from the group consisting of 6-(2,6-difluoro-4-(7-((1-(isopropylsulfonyl)azetidin-3-yl)oxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.188. The compound of E30 selected from the group consisting of 6-(2,6-difluoro-4-(2-(methyl-d₃)-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.189. The compound of E30 selected from the group consisting of 6-(4-(7-((1-cyclohexylazetidin-3-yl)oxy)-2-methyl-2H-indazol-4-yl)-2,6-difluorobenzyl)-6,7-dihydro-5H-pyrrolo[3,4-b]pyridin-5-one-7,7-d₂, or a pharmaceutically acceptable salt thereof.

E30.190. The compound of E30 selected from the group consisting of 6-(2-cyclopropyl-6-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.191. The compound of E30 selected from the group consisting of 6-(2-cyclopropyl-6-fluoro-4-(3-fluoro-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.192. The compound of E30 selected from the group consisting of 6-(2-cyclopropyl-6-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.193. The compound of E30 selected from the group consisting of 6-(2-cyclopropyl-6-fluoro-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.194. The compound of E30 selected from the group consisting of 6-(2-fluoro-6-methyl-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.195. The compound of E30 selected from the group consisting of 6-(2-fluoro-4-(7-methoxy-2-methyl-2H-indazol-4-yl)-6-methylbenzyl)-6,7-dihydro-5H-pyrrolo[3,4-b]pyridin-5-one-7,7-d₂, or a pharmaceutically acceptable salt thereof.

E30.196. The compound of E30 selected from the group consisting of 6-(4-(7-cyclopropoxy-2-methyl-2H-indazol-4-yl)-2-fluoro-6-methylbenzyl)-6,7-dihydro-5H-pyrrolo[3,4-b]pyridin-5-one-7,7-d₂, or a pharmaceutically acceptable salt thereof.

E30.197. The compound of E30 selected from the group consisting of 6-(4-(2,7-dimethyl-2H-indazol-4-yl)-2-fluoro-6-methylbenzyl)-6,7-dihydro-5H-pyrrolo[3,4-b]pyridin-5-one-7,7-d₂, or a pharmaceutically acceptable salt thereof.

E30.198. The compound of E30 selected from the group consisting of 6-(2-fluoro-4-(7-fluoro-2-methyl-2H-indazol-4-yl)-6-methylbenzyl)-6,7-dihydro-5H-pyrrolo[3,4-b]pyridin-5-one-7,7-d₂, or a pharmaceutically acceptable salt thereof.

E30.199. The compound of E30 selected from the group consisting of 6-(4-(7-chloro-2-methyl-2H-indazol-4-yl)-2-fluoro-6-methylbenzyl)-6,7-dihydro-5H-pyrrolo[3,4-b]pyridin-5-one-7,7-d₂, or a pharmaceutically acceptable salt thereof.

E30.200. The compound of E30 selected from the group consisting of 6-(2-fluoro-6-methyl-4-(2-methyl-7-(trifluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.201. The compound of E30 selected from the group consisting of 6-(2-fluoro-6-methyl-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.202. The compound of E30 selected from the group consisting of 6-(4-(7-cyclopropoxy-2-methyl-2H-indazol-4-yl)-2-cyclopropyl-6-fluorobenzyl)-6,7-dihydro-5H-pyrrolo[3,4-b]pyridin-5-one-7,7-d₂, or a pharmaceutically acceptable salt thereof.

E30.203. The compound of E30 selected from the group consisting of 6-(2-cyclopropyl-6-fluoro-4-(7-(2-methoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.204. The compound of E30 selected from the group consisting of 6-(2-cyclopropyl-4-(2,7-dimethyl-2H-indazol-4-yl)-6-fluorobenzyl)-6,7-dihydro-5H-pyrrolo[3,4-b]pyridin-5-one-7,7-d₂, or a pharmaceutically acceptable salt thereof.

E30.205. The compound of E30 selected from the group consisting of 6-(2-cyclopropyl-6-fluoro-4-(7-fluoro-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.206. The compound of E30 selected from the group consisting of 6-(4-(7-chloro-2-methyl-2H-indazol-4-yl)-2-cyclopropyl-6-fluorobenzyl)-6,7-dihydro-5H-pyrrolo[3,4-b]pyridin-5-one-7,7-d₂, or a pharmaceutically acceptable salt thereof.

E30.207. The compound of E30 selected from the group consisting of 6-(2-cyclopropyl-6-fluoro-4-(2-methyl-7-(trifluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.208. The compound of E30 selected from the group consisting of 6-(2-cyclopropyl-6-fluoro-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.208. The compound of E30 selected from the group consisting of 6-(2-cyclopropyl-6-fluoro-4-(7-methoxy-2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.210. The compound of E30 selected from the group consisting of 6-(2-fluoro-6-isobutoxy-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.211. The compound of E30 selected from the group consisting of 6-(2-fluoro-6-isobutoxy-4-(2-methyl-2H-indazol-5-yl)benzyl)-6,7-dihydro-5H-pyrrolo[3,4-b]pyridin-5-one-7,7-d₂, or a pharmaceutically acceptable salt thereof.

E30.212. The compound of E30 selected from the group consisting of 6-(4-(7-cyclopropoxy-2-methyl-2H-indazol-4-yl)-2-fluoro-6-isobutoxybenzyl)-6,7-dihydro-5H-pyrrolo[3,4-b]pyridin-5-one-7,7-d₂, or a pharmaceutically acceptable salt thereof.

E30.213. The compound of E30 selected from the group consisting of 6-(4-(2,7-dimethyl-2H-indazol-4-yl)-2-fluoro-6-isobutoxybenzyl)-6,7-dihydro-5H-pyrrolo[3,4-b]pyridin-5-one-7,7-d₂, or a pharmaceutically acceptable salt thereof.

E30.214. The compound of E30 selected from the group consisting of 6-(2-fluoro-4-(7-fluoro-2-methyl-2H-indazol-4-yl)-6-isobutoxybenzyl)-6,7-dihydro-5H-pyrrolo[3,4-b]pyridin-5-one-7,7-d₂, or a pharmaceutically acceptable salt thereof.

E30.215. The compound of E30 selected from the group consisting of 6-(4-(7-chloro-2-methyl-2H-indazol-4-yl)-2-fluoro-6-isobutoxybenzyl)-6,7-dihydro-5H-pyrrolo[3,4-b]pyridin-5-one-7,7-d₂, or a pharmaceutically acceptable salt thereof.

E30.216. The compound of E30 selected from the group consisting of 6-(2-fluoro-6-isobutoxy-4-(2-methyl-7-(trifluoromethyl)-2H-indazol-4-yl)benzyl)-6,7-dihydro-5H-pyrrolo[3,4-b]pyridin-5-one-7,7-d₂, or a pharmaceutically acceptable salt thereof.

E30.217. The compound of E30 selected from the group consisting of 6-(2-fluoro-6-isobutoxy-4-(2-methyl-2H-pyrazolo[3,4-b]pyridin-4-yl)benzyl)-6,7-dihydro-5H-pyrrolo[3,4-b]pyridin-5-one-7,7-d₂, or a pharmaceutically acceptable salt thereof.

E30.218. The compound of E30 selected from the group consisting of 6-(2-fluoro-6-isobutoxy-4-(7-(2-methoxyethoxy)-2-methyl-2H-indazol-4-yl)benzyl)-6,7-dihydro-5H-pyrrolo[3,4-b]pyridin-5-one-7,7-d₂, or a pharmaceutically acceptable salt thereof.

E30.219. The compound of E30 selected from the group consisting of 6-(2-chloro-6-cyclopropyl-4-(2-methyl-2H-indazol-4-yl)benzyl)-6,7-dihydro-5H-pyrrolo[3,4-]pyridin-5-one-7,7-d₂, or a pharmaceutically acceptable salt thereof.

E30.220. The compound of E30 selected from the group consisting of 6-(2-chloro-6-cyclopropyl-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.221. The compound of E30 selected from the group consisting of 6-(2-(azetidin-1-yl)-6-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.222. The compound of E30 selected from the group consisting of 6-(2-(azetidin-1-yl)-6-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.223. The compound of E30 selected from the group consisting of 6-(2-cyclopentyl-6-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.224. The compound of E30 selected from the group consisting of 6-(2-cyclopentyl-6-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.225. The compound of E30 selected from the group consisting of 6-(2-fluoro-4-(2-methyl-2H-indazol-4-yl)-6-(trifluoromethyl)benzyl)-6,7-dihydro-5H-pyrrolo[3,4-b]pyridin-5-one-7,7-d₂, or a pharmaceutically acceptable salt thereof.

E30.226. The compound of E30 selected from the group consisting of 6-(2-fluoro-4-(2-(methyl-d₃)-2H-indazol-4-yl)-6-(trifluoromethyl)benzyl)-6,7-dihydro-5H-pyrrolo[3,4-b]pyridin-5-one-7,7-d₂, or a pharmaceutically acceptable salt thereof.

E30.227. The compound of E30 selected from the group consisting of 6-(2,6-dichloro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.228. The compound of E30 selected from the group consisting of 6-(2,6-dichloro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.229. The compound of E30 selected from the group consisting of 6-(2-(dimethylamino)-6-fluoro-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.230. The compound of E30 selected from the group consisting of 6-(2-(dimethylamino)-6-fluoro-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.231. The compound of E30 selected from the group consisting of 6-(2-fluoro-6-methoxy-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.232. The compound of E30 selected from the group consisting of 6-(2-fluoro-6-methoxy-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.233. The compound of E30 selected from the group consisting of 6-(2-isopropyl-4-(2-methyl-2H-indazol-4-yl)benzyl)-6,7-dihydro-5H-pyrrolo[3,4-b]pyridin-5-one-7,7-d₂, or a pharmaceutically acceptable salt thereof.

E30.234. The compound of E30 selected from the group consisting of 6-(2-isopropyl-4-(2-(methyl-d₃)-2H-indazol-4-yl)benzyl)-6,7-dihydro-5H-pyrrolo[3,4-b]pyridin-5-one-7,7-d₂, or a pharmaceutically acceptable salt thereof.

E30.235. The compound of E30 selected from the group consisting of 6-(2,6-difluoro-4-(2-methylpyrazolo[1,5-a]pyridin-4-yl)benzyl)-6,7-dihydro-5H-pyrrolo[3,4-b]pyridin-5-one-7,7-d₂, or a pharmaceutically acceptable salt thereof.

E30.236. The compound of E30 selected from the group consisting of 6-(2,6-difluoro-4-(2-(methyl-d₃)pyrazolo[1,5-a]pyridin-4-yl)benzyl)-6,7-dihydro-5H-pyrrolo[3,4-b]pyridin-5-one-7,7-d₂, or a pharmaceutically acceptable salt thereof.

E30B. The compound of E1.1, E1.2, or E1.3 selected from the group consisting of 3-bromo-6-(2,6-difluoro-4-(2-methyl-2H-indazol-4-yl)benzyl)-7-hydroxy-6,7-dihydro-5H-pyrrolo[3,4-b]pyridin-5-one-7-d, or a pharmaceutically acceptable salt thereof.

E31. The compound of any of E1-E30.236, or a pharmaceutically acceptable salt thereof, having a positive allosteric modulation of the mAChR M₁ response to acetylcholine with EC₅₀ of less than or equal to 0.5 μM.

E31.1. The compound of E31 selected from the group consisting of
6-(2,6-difluoro-4-(2-methyl-2H-indazol-4-yl)benzyl)-6,7-dihydro-5H-pyrrolo[3,4-b]pyridin-5-one-7,7-d₂;
6-(4-(2,7-dimethyl-2H-indazol-4-yl)-2,6-difluorobenzyl)-6,7-dihydro-5H-pyrrolo[3,4-b]pyridin-5-one-7,7-d₂;
6-(4-(2,7-dimethyl-2H-indazol-4-yl)-2-fluorobenzyl)-6,7-dihydro-5H-pyrrolo[3,4-b]pyridin-5-one-7,7-d₂;
6-(2,6-difluoro-4-(2-(methyl-d₃)-2H-indazol-4-yl)benzyl)-6,7-dihydro-5H-pyrrolo[3,4-b]pyridin-5-one-7,7-d₂;
6-(2-methyl-4-(2-methyl-2H-indazol-5-yl)benzyl)-6,7-dihydro-5H-pyrrolo[3,4-b]pyridin-5-one-7,7-d₂;
6-(2,6-difluoro-4-(7-fluoro-2-methyl-2H-indazol-4-yl)benzyl)-6,7-dihydro-5H-pyrrolo[3,4-b]pyridin-5-one-7,7-d₂;
6-(2,6-difluoro-4-(7-methoxy-2H-indazol-4-yl)benzyl)-6,7-dihydro-5H-pyrrolo[3,4-b]pyridin-5-one-7,7-d₂;
6-(2,6-difluoro-4-(7-methoxy-2-methyl-2H-indazol-4-yl)benzyl)-6,7-dihydro-5H-pyrrolo[3,4-b]pyridin-5-one-7,7-d₂
6-(4-(7-chloro-2-methyl-2H-indazol-4-yl)-2,6-difluorobenzyl)-6,7-dihydro-5H-pyrrolo[3,4-b]pyridin-5-one-7,7-d₂;
6-(4-(2-ethyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-(fluoromethyl)-2H-indazol-4-yl)benzyl)-6,7-dihydro-5H-pyrrolo[3,4-b]pyridin-5-one-7,7-d₂;
6-(2,6-difluoro-4-(7-fluoro-2-(methyl-d₃)-2H-indazol-4-yl)benzyl)-6,7-dihydro-5H-pyrrolo[3,4-b]pyridin-5-one-7,7-d₂;
6-(4-(7-fluoro-2-methyl-2H-indazol-4-yl)-2-methylbenzyl)-6,7-dihydro-5H-pyrrolo[3,4-b]pyridin-5-one-7,7-d₂;
6-(4-(2-(difluoromethyl)-2H-indazol-4-yl)-2,6-difluorobenzyl)-6,7-dihydro-5H-pyrrolo[3,4-b]pyridin-5-one-7,7-d₂;
6-(2,6-difluoro-4-(7-methoxy-2-(methyl-d₃)-2H-indazol-4-yl)benzyl)-6,7-dihydro-5H-pyrrolo[3,4-b]pyridin-5-one-7,7-d₂;
6-(2,6-difluoro-4-(7-methyl-2-(methyl-d₃)-2H-indazol-4-yl)benzyl)-6,7-dihydro-5H-pyrrolo[3,4-b]pyridin-5-one-7,7-d₂;
6-(2-fluoro-4-(2-(methyl-d₃)-2H-indazol-4-yl)benzyl)-6,7-dihydro-5H-pyrrolo[3,4-b]pyridin-5-one-7,7-d₂;
6-(2-fluoro-4-(7-fluoro-2-(methyl-d₃)-2H-indazol-4-yl)benzyl)-6,7-dihydro-5H-pyrrolo[3,4-b]pyridin-5-one-7,7-d₂;
6-(2-fluoro-4-(7-methoxy-2-(methyl-d₃)-2H-indazol-4-yl)benzyl)-6,7-dihydro-5H-pyrrolo[3,4-b]pyridin-5-one-7,7-d₂;
6-(2-fluoro-4-(7-methyl-2-(methyl-d₃)-2H-indazol-4-yl)benzyl)-6,7-dihydro-5H-pyrrolo[3,4-b]pyridin-5-one-7,7-d₂;
6-(4-(7-methoxy-2-(methyl-d₃)-2H-indazol-4-yl)-2-methylbenzyl)-6,7-dihydro-5H-pyrrolo[3,4-b]pyridin-5-one-7,7-d₂;
6-(2,6-difluoro-4-(6-fluoro-2-methyl-2H-indazol-4-yl)benzyl)-6,7-dihydro-5H-pyrrolo[3,4-b]pyridin-5-one-7,7-d₂;
6-(2,6-difluoro-4-(2-methyl-2H-indazol-4-yl)benzyl)-3-hydroxy-6,7-dihydro-5H-pyrrolo[3,4-b]pyridin-5-one-7,7-d₂;
6-(2,6-difluoro-4-(2-methyl-2H-indazol-4-yl)benzyl)-3-(methoxy-d₃)-6,7-dihydro-5H-pyrrolo[3,4-b]pyridin-5-one-7,7-d₂;
6-(4-(6-fluoro-2-methyl-2H-indazol-4-yl)benzyl)-6,7-dihydro-5H-pyrrolo[3,4-b]pyridin-5-one-7,7-d₂;
6-(4-(7-chloro-2-(methyl-d₃)-2H-indazol-4-yl)-2,6-difluorobenzyl)-6,7-dihydro-5H-pyrrolo[3,4-b]pyridin-5-one-7,7-d₂;
6-(2,6-difluoro-4-(7-methoxy-2-methyl-2H-indazol-5-yl)benzyl)-6,7-dihydro-5H-pyrrolo[3,4-b]pyridin-5-one-7,7-d₂; and
6-(2,6-difluoro-4-(2-(methyl-d₃)-2H-indazol-4-yl)benzyl)-3-(methoxy-d₃)-6,7-dihydro-5H-pyrrolo[3,4-b]pyridin-5-one-7,7-d₂;
   or a pharmaceutically acceptable salt thereof.

E32. A compound of any of E1-E31.1, or a pharmaceutically acceptable salt thereof, that has at least 50% deuterium incorporation at each deuterium label.

E32.1. The compound of E32, or pharmaceutically acceptable salt thereof, that has at least 75% deuterium incorporation at each deuterium-label.

E32.2. The compound of E32, or pharmaceutically acceptable salt thereof, that has at least 90% deuterium incorporation at each deuterium-label.

E32.3. The compound of E32, or pharmaceutically acceptable salt thereof, that has at least 99% deuterium incorporation at each deuterium-label.

E32.4. The compound of E32, or pharmaceutically acceptable salt thereof, that has at least 99.5% deuterium incorporation at each deuterium-label.

E33. A hydrate, solvate, polymorph, or prodrug of the compound of any of E1-E28.4, or a pharmaceutically acceptable salt thereof.

E34. A pharmaceutical composition comprising the compound, hydrate, solvate, polymorph, or prodrug of any of E1-E33, or pharmaceutically acceptable salt thereof, and a pharmaceutically acceptable carrier.

E35 A composition comprising:
(a) a compound of any of E1, E1.2-E1.4, E2-E30.236, or E31-E32.4 according to formula (I), or a pharmaceutically acceptable salt, hydrate, solvate, polymorph, or prodrug thereof, and
(b) a compound of any of E1.1-E29.1,E30B, or E32-E32.4 according to formula (II), or a pharmaceutically acceptable salt, hydrate, solvate, polymorph, or prodrug thereof.

E35.1. The composition of E35, further comprising a pharmaceutically acceptable carrier.

E36. A method for the treatment of a disorder associated with muscarinic acetylcholine receptor activity in a mammal, comprising administering to the mammal an effective amount of the compound, hydrate, solvate, polymorph, or prodrug of any of E1-E33, or a pharmaceutically acceptable salt thereof, or the composition of any of E34-E35.1.

E37. The method of E36, wherein the mammal is human.

E38. The method of E36 or E37, wherein the muscarinic acetylcholine receptor is mAChR M₁.

E39. The method of any of E36-E38, wherein the mammal has been diagnosed with a need for treatment of the disorder prior to the administering step.

E40. The method of any of E36-E39, further comprising the step of identifying a mammal in need of treatment of the disorder.

E41. The method of any of E36-E40, wherein the disorder is a neurological disorder or psychiatric disorder, or a combination thereof.

E42. The method of any of E36-E41, wherein the disorder is psychosis, schizophrenia, conduct disorder, disruptive behavior disorder, bipolar disorder, psychotic episodes of anxiety, anxiety associated with psychosis, psychotic mood disorders, severe major depressive disorder, mood disorders associated with psychotic disorders, acute mania, depression associated with bipolar disorder, mood disorders associated with schizophrenia, behavioral manifestations of mental retardation, conduct disorder, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, or memory disorders, or a combination thereof.

E43. The method of any of E36-E41, wherein the disorder is Alzheimer's disease, schizophrenia, a sleep disorder, a pain disorder, or a cognitive disorder, or a combination thereof.

E44. The method of E43, wherein the pain disorder is neuropathic pain, central pain syndrome, postsurgical pain syndrome, bone and joint pain, repetitive motion pain, dental pain, cancer pain, myofascial pain, perioperative pain, chronic pain, dysmennorhea, inflammatory pain, headache, migraine headache, cluster headache, headache, primary hyperalgesia, secondary hyperalgesis, primary allodynia, secondary allodynia, or a combination thereof.

E45. The compound, hydrate, solvate, polymorph, or prodrug of any of E1-E33, or a pharmaceutically acceptable salt thereof, or the composition of any of E34-E35.1, for use in the treatment of a disorder associated with muscarinic acetylcholine receptor activity in a mammal.

E46. Use of the compound, hydrate, solvate, polymorph, or prodrug of any of E1-E33, or a pharmaceutically acceptable salt thereof, or the composition of any of E34-E35.1, for the preparation of a medicament for the treatment of a disorder associated with muscarinic acetylcholine receptor activity in a mammal.

Compound names and/or structures can be assigned/determined by using the Struct=Name naming algorithm as part of CHEMDRAW®, versions 19.1 or 20.0.

Compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. In the compounds of formula (I) or (II) when no specific configuration is indicated at a stereogenic center (e.g., carbon), the compounds include all possible stereoisomers.

Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

In the compounds of formula (I) or (II), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I) or (II), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. Pharmaceutical Compositions

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I) or (II)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I) or (II), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I) or (II)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I) or (II)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I) or (II)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

a. Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the disclosure may provide a spray-dried dispersion formulation comprising a compound of formula (I) or (II).

### 4. Therapeutic Uses and Methods

The disclosed compounds are positive allosteric modulators of mAChR M₁. Thus, by positive allosteric modulation, the compounds indirectly activate the muscarinic receptor subtype M₁. In one aspect, the disclosed compounds potentiate the agonist response (e.g., acetylcholine) of mAChR M₁. In a further aspect, the disclosed compounds increase mAChR M₁ response to non-maximal concentrations of agonist in the presence of compound compared to the response to agonist in the absence of compound. The potentiation of mAChR M₁ activity can be demonstrated by methodology known in the art. For example, activation of mAChR M₁ activity can be determined by measurement of calcium flux in response to agonist, e.g. acetylcholine, in cells loaded with a Ca²⁺-sensitive fluorescent dye (e.g., Fluo-4). In a further aspect, the calcium flux was measured as an increase in fluorescent static ratio. In a yet further aspect, positive allosteric modulator activity was analyzed as a concentration-dependent increase in the EC₂₀ acetylcholine response (i.e. the response of mAChR M₁ at a concentration of acetylcholine that yields 20% of the maximal response).

In an embodiment, the disclosed compounds may activate mAChR M₁ response as an increase in calcium fluorescence in mAChR M₁-transfected CHO-K1 cells in the presence of the compound, compared to the response of equivalent CHO-K1 cells in the absence of the compound. For example, a disclosed compound may have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM. In an embodiment, the mAChR M₁-transfected CHO-K1 cells are transfected with human mAChR M₁. In another embodiment, the mAChR M₁-transfected CHO-K1 cells are transfected with rat mAChR M₁.

In an embodiment, the disclosed compounds exhibit weak or substantially no agonist activation of mAChR M₁ response (i.e., lack of activation in the absence of a known agonist such as acetylcholine). Lack of agonist activity may be measured as weak or no increase in calcium fluorescence in mAChR M₁-transfected CHO-K1 cells in the presence of the compound, compared to the response of equivalent CHO-K1 cells in the absence of the compound. Lack of mAChR M₁ agonist activity may be determined as a percent response relative to acetylcholine. For example, a disclosed compound may have less than or equal to 30%, 25%, 20%, 15%, 10%, 5%, or 1% mAChR M₁ agonist activity relative to acetylcholine. A disclosed compound may have substantially no mAChR M₁ agonist activity. In further embodiments, the disclosed compounds exhibit positive allosteric modulation of mAChR M₁ response to acetylcholine, as described herein, at concentrations that have weak or substantially no agonist activity, as described herein. The absence of mAChR M₁ agonist activity may contribute to the avoidance of cholinergic adverse effect liability.

In an embodiment, the disclosed compounds exhibit potentiation of mAChR M₁ response to acetylcholine as an increase in response to non-maximal concentrations of acetylcholine in CHO-K1 cells transfected with a mammalian mAChR M₁ in the presence of the compound, compared to the response to acetylcholine in the absence of the compound. For example, CHO-K1 cells can be transfected with human mAChR M₁. For example, CHO-K1 cells can be transfected with rat mAChR M₁. For example, a compound can exhibit positive allosteric modulation of mAChR M₁ with an EC₅₀ of less than or equal to 10 μM, less than or equal to 5 μM, less than or equal to 2.5 μM, less than or equal to 1 μM, less than or equal to 500 nM, less than or equal to 250 nM, or less than or equal to 100 nM. Alternatively, the disclosed compounds exhibit potentiation of mAChR M₁ response to acetylcholine as an increase in response to non-maximal concentrations of acetylcholine in CHO-K1 cells transfected with human mAChR M₁ in the presence of the compound, compared to the response to acetylcholine in the absence of the compound. For example, a compound can exhibit positive allosteric modulation of mAChR M₁ with an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM.

In an embodiment, the disclosed compounds exhibit positive allosteric modulation of mAChR M₁ response to acetylcholine as an increase in response to non-maximal concentrations of acetylcholine in CHO-K1 cells transfected with a mAChR M₁ in the presence of the compound, compared to the response to acetylcholine in the absence of the compound. For example, the disclosed compounds may exhibit positive allosteric modulation of the mAChR M₁ response to acetylcholine with an EC₅₀ of less than or equal to 10 μM, less than or equal to 5 μM, less than or equal to 2.5 μM, less than or equal to 1 μM, less than or equal to 500 nM, less than or equal to 250 nM, or less than or equal to 100 nM. In an embodiment, the EC₅₀ for positive allosteric modulation is determined in CHO-K1 cells are transfected with a mAChR M₁. In another embodiment, the CHO-K1 cells are transfected with a human mAChR M₁. In another embodiment, the CHO-K1 cells are transfected with a rat mAChR M₁.

Positive allosteric modulation of the mAChR M₁ response to acetylcholine with EC₅₀ of less than or equal to 0.5 μM, less than or equal to 0.25 μM, or less than or equal to 0.1 μM are all considered potent positive allosteric modulating activity.

In an embodiment, the compounds activate mAChR M₁ response in mAChR M₁-transfected CHO-K1 cells with an EC₅₀ less than the EC₅₀ for one or more of mAChR M₂, mAChR M₃, mAChR M₄, or mAChR M₅ response in mAChR M₂, M₃, M₄ or M₅-transfected CHO-K1 cells. That is, the disclosed compounds can have selectivity for the mAChR M₁ receptor vis-à-vis one or more of the mAChR M₂, M₃, M₄ or M₅ receptors. For example, the disclosed compounds can activate mAChR M₁ response with an EC₅₀ of at least 5-fold less than that for mAChR M₂, at least 10-fold less than that for mAChR M₂, at least 20-fold less than that for mAChR M₂, at least 30-fold less than that for mAChR M₂, at least 50-fold less than that for mAChR M₂, or at least 100-fold less than that for mAChR M₂. In another embodiment, the disclosed compounds can activate mAChR M₁ response with an EC₅₀ of at least 5-fold less than that for mAChR M₃, at least 10-fold less than that for mAChR M₃, at least 20-fold less than that for M₃, at least 30-fold less than that for mAChR M₃, at least 50-fold less than that for mAChR M₃, or at least 100-fold less than that for mAChR M₃. In another embodiment, the disclosed compounds can activate mAChR M₁ response with an EC₅₀ of at least 5-fold less than that for mAChR M₄, at least 10-fold less than that for mAChR M₄, at least 20-fold less than that for M₄, at least 30-fold less than that for mAChR M₄, at least 50-fold less than that for mAChR M₄, or at least 100-fold less than that for mAChR M₄. In another embodiment, the disclosed compounds can activate mAChR M₁ response with an EC₅₀ of at least 5-fold less than that for mAChR M₅, at least 10-fold less than that for mAChR M₅, at least 20-fold less than that for mAChR M₅, at least 30-fold less than that for mAChR M₅, at least 50-fold less than that for mAChR M₅, or at least 100-fold less than that for mAChR M₅. In another embodiment, the disclosed compounds can activate mAChR M₁ response with an EC₅₀ of at least 5-fold less than that for the mAChR M₂, M₃, M₄ or M₅ receptors, at least 10-fold less than that for the mAChR M₂, M₃, M₄ or M₅ receptors, at least 20-fold less than that for the mAChR M₂, M₃, M₄ or M₅ receptors, at least 30-fold less than that for the mAChR M₂, M₃, M₄ or M₅ receptors, at least 50-fold less than that for the mAChR M₂, M₃, M₄ or M₅ receptors, or at least 100-fold less than that for the mAChR M₂, M₃, M₄ or M₅ receptors. In another embodiment, the compound activates mAChR M₁ response in mAChR M₁-transfected CHO-K1 cells and is inactive for one or more of mAChR M₁, mAChR M₃, mAChR M₄, or mAChR M₅ response in mAChR M₂, M₃, M₄ or M₅-transfected CHO-K1 cells.

In an embodiment, the compounds activate mAChR M₁ response in M₁-transfected CHO-K1 cells with an EC₅₀ of less than or equal to 10 μM and exhibits a selectivity for the M₁ receptor vis-à-vis one or more of the mAChR M₂, M₃, M₄ or M₅ receptors. For example, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₂, at least 10-fold less than that for mAChR M₂, at least 20-fold less than that for mAChR M₂, at least 30-fold less than that for mAChR M₂, or at least 50-fold less than that for mAChR M₂. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₃, at least 10-fold less than that for mAChR M₃, at least 20-fold less than that for mAChR M₃, at least 30-fold less than that for mAChR M₃, or at least 50-fold less than that for mAChR M₃. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₄, of at least 10-fold less than that for mAChR M₄, of at least 20-fold less than that for mAChR M₄, of at least 30-fold less than that for mAChR M₄, or at least 50-fold less than that for mAChR M₄. In another embodiment, the compound can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for mAChR M₅, of at least 10-fold less than that for mAChR M₅, of at least 20-fold less than that for mAChR M₅, of at least 30-fold less than that for mAChR M₅, or at least 50-fold less than that for mAChR M₅. In another embodiment, the compounds can have an EC₅₀ of less than or equal to 10 μM, less than or equal to 5 μM, less than or equal to 2.5 μM, less than or equal to 1 μM, less than or equal to 500 nM, less than or equal to 250 nM, less than or equal to 100 nM, or less than or equal to 50 nM; and the compounds can also activate mAChR M₁ response with an EC₅₀ of at least 5-fold less than that for the mAChR M₂, M₃, M₄ or M₅ receptors, at least 10-fold less than that for the mAChR M₂, M₃, M₄ or M₅ receptors, at least 20-fold less than that for the mAChR M₂, M₃, M₄ or M₅ receptors, at least 30-fold less than that for the mAChR M₂, M₃, M₄ or M₅ receptors, or at least 50-fold less than that for the mAChR M₂, M₃, M₄ or M₅ receptors.

The disclosed compounds may be used in methods for treatment of mAChR M₁ related medical disorders and/or diseases. The methods of treatment may comprise administering to a subject in need of such treatment a composition comprising a therapeutically effective amount of the compound of formula (I) or (II).

The compounds can be administered to a subject in need thereof to modulate mAChR M₁, for a variety of diverse biological processes. The present disclosure is directed to methods for administering the composition to potentiate mAChR M₁, a GPCR whose dysfunction is associated with neurological and psychiatric disorders, for example.

The compounds may be useful for treating and preventing certain diseases and disorders in humans and animals related to mAChR M₁ dysfunction. Treatment or prevention of such diseases and disorders can be effected by modulating mAChR M₁ in a subject, by administering a compound or composition of the disclosure, either alone or in combination with another active agent as part of a therapeutic regimen to a subject in need thereof.

In combination therapy, the other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

In an embodiment, the compounds can be coadministered with anti-Alzheimer's agents, beta-secretase inhibitors, gamma-secretase inhibitors, orthosteric muscarinic agonists, muscarinic potentiators, cholinesterase inhibitors, HMG-CoA reductase inhibitors, NSAIDs and anti-amyloid antibodies. In a further aspect, the compounds can be administered in combination with sedatives, hypnotics, anxiolytics, antipsychotics (typical and atypical), selective serotonin reuptake inhibitors (SSRIs), monoamine oxidase inhibitors (MAOIs), 5-HT2 antagonists, GlyT1 inhibitors and the like such as, but not limited to: risperidone, clozapine, haloperidol, fluoxetine, prazepam, xanomeline, lithium, phenobarbitol, and salts thereof and combinations thereof.

The compounds may be useful for treating a disease or disorder associated with dysfunction of mAChR M₁, wherein the disease or disorder is selected from at least one of Alzheimer's disease, a sleep disorder, a pain disorder, a cognitive disorder, psychosis, schizophrenia, conduct disorder, disruptive behavior disorder, bipolar disorder, psychotic episodes of anxiety, anxiety associated with psychosis, psychotic mood disorders, severe major depressive disorder, mood disorders associated with psychotic disorders, acute mania, depression associated with bipolar disorder, mood disorders associated with schizophrenia, behavioral manifestations of mental retardation, conduct disorder, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

The compounds may be useful for treating a pain disorder, wherein the pain disorder is neuropathic pain, central pain syndrome, postsurgical pain syndrome, bone and joint pain, repetitive motion pain, dental pain, cancer pain, myofascial pain, perioperative pain, chronic pain, dysmennorhea, inflammatory pain, headache, migraine headache, cluster headache, headache, primary hyperalgesia, secondary hyperalgesis, primary allodynia, secondary allodynia, or a combination thereof.

The compounds disclosed herein are useful for treating, preventing, ameliorating, controlling or reducing the risk of a variety of disorders wherein the patient or subject would benefit from selective positive allosteric modulation of the M₁ receptor. In one aspect, a treatment can include selective M₁ receptor modulation to an extent effective to affect cholinergic activity. Thus, a disorder can be associated with cholinergic activity, for example cholinergic hypofunction. In one aspect, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which muscarinic receptor activation is predicted to be beneficial, in a subject comprising the step of administering to the subject at least one disclosed compound; at least one disclosed pharmaceutical composition; and/or at least one disclosed product in a dosage and amount effective to treat the disorder in the subject.

The disclosure is directed to the use of described chemical compositions to treat diseases or disorders in patients (preferably human) wherein muscarinic receptor activation would be predicted to have a therapeutic effect, such as Alzheimer's disease (both palliative cognitive and disease-modifying), cognitive impairment, schizophrenia, pain disorders (including acute pain, neuropathic pain and inflammatory pain), and sleep disorders, by administering one or more disclosed compounds or products.

Also provided is a method for the treatment of a disorder in a mammal comprising the step of administering to the mammal at least one disclosed compound, composition, or medicament.

a. Neurological and Psychiatric Disorders

The disclosed compounds have utility in treating a variety of neurological and psychiatric disorders, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosis psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age-related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, post-encephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de La Tourette's syndrome, epilepsy, muscular spasms and disorders associated with muscular spasticity or weakness including tremors; dyskinesias including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics),and dystonia (including generalized dystonia such as idiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

### 1. Cognitive Disorders

The present disclosure provides a method for treating cognitive disorders, comprising: administering to a patient in need thereof an effective amount of a compound of the present disclosure. Particular cognitive disorders are dementia, delirium, amnestic disorders and age-related cognitive decline. The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

### 2. Anxiety Disorders

The present disclosure provides a method for treating anxiety disorders, comprising: administering to a patient in need thereof an effective amount of a compound of the present disclosure. Particular anxiety disorders are generalized anxiety disorder, obsessive-compulsive disorder and panic attack. The text revision of the fourth edition of the Diagnostic and Statistical Manual of Mental Disorders (DSM-IV-TR) (2000, American Psychiatric Association, Washington DC) provides a diagnostic tool that includes anxiety disorders are generalized anxiety disorder, panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

### 3. Alzheimer's Disease

Alzheimer's disease (AD) is a neurodegenerative disease affecting the elderly, which results in progressive impairment of memory, language skills and severe behavioral deficits. Hallmarks of the disease include degeneration of cholinergic neurons in the cerebral cortex, hippocampus, basal forebrain and other regions of the brain important for memory and cognition. Other hallmarks of AD include neurofibrillary tangles composed of hyperphosphorylated tau and accumulation of amyloid β peptide (Aβ). Aβ is a 39-43 amino acid peptide produced in the brain by proteolytic processing of β-amyloid precursor protein (APP) by the β-amyloid cleaving enzyme (BACE) and gamma secretase which leads to accumulation of Aβ in the brain, where Aβ 1-40 and 1-42 are the principal aggregate-forming species of AD.

Activation of various muscarinic receptors, particularly the M₁ subtype, has been proposed as a mechanism to enhance cognition in disorders such as AD. Thus, without wishing to be bound by theory, it is believed that selective positive allosteric modulators of mAChR subtypes that regulate processes involved in cognitive function could prove superior to AChE inhibitors for treatment of AD and related disorders as it is postulated that these compounds would exhibit improved selectivity for specific mAChRs.

Phase III clinical trials have shown that orthosteric mAChR activators can have efficacy in improving cognitive performance in AD patients. Moreover, data indicate that administration of M₁ activators decreases behavioral disturbances, including delusions, hallucinations, outbursts, and other symptoms in patients suffering from neurodegenerative diseases such as Alzheimer's disease. However, dose limiting adverse effects that may be due to lack of mAChR M₁ selectivity led to failed launches of previous M₁ agonists. In some cases, evidence suggests that mAChR activation also has the potential to be disease-modifying in that these agents may lower Aβ in AD patients. The M₁-selective allosteric agonist TBPB was found to display effects on the processing of APP toward the non-amyloidogenic pathway and decrease Aβ 1-40 and 1-42 production in vitro. These data suggest that selective activation of M₁ may provide a novel approach for both symptomatic and disease modifying the treatment of Alzheimer's disease.

### 4. Schizophrenia

Schizophrenia is a debilitating psychiatric disorder characterized by a combination of negative (blunted affect, withdrawal, anhedonia) and positive (paranoia, hallucinations, delusions) symptoms as well as marked cognitive deficits. While schizophrenia remains an idiopathic disorder, it appears to be produced by a complex interaction of biological, environmental, and genetic factors. Over 40 years ago it was found that phencyclidine (PCP) induces a psychotic state in humans that is very similar to that observed in schizophrenic patients. The finding that the main mode of action of PCP is that of a non-competitive antagonist of the N-methyl-D-aspartate (NMDA) subtype of ionotropic glutamate receptor stimulated a series of studies that have led to the development of the NMDA receptor hypofunction model of schizophrenia.

The present disclosure provides a method for treating schizophrenia or psychosis comprising: administering to a patient in need thereof an effective amount of a compound of the present disclosure. Particular schizophrenia or psychosis pathologies are paranoid, disorganized, catatonic or undifferentiated schizophrenia and substance-induced psychotic disorder. The text revision of the fourth edition of the Diagnostic and Statistical Manual of Mental Disorders (DSM-IV-TR) (2000, American Psychiatric Association, Washington DC) provides a diagnostic tool that includes paranoid, disorganized, catatonic or undifferentiated schizophrenia and substance-induced psychotic disorder.

NMDA receptor function can be modulated by activation of G Protein-Coupled Receptors (GPCRs) that are known to physically and/or functionally interact with the NMDA receptor. The NMDA receptor hypofunction hypothesis is a proposal to explain the underlying cause of schizophrenia. According to this hypothesis, any agent that can potentiate NMDA receptor currents, either directly by action on modulatory sites on the NMDA receptor (e.g., the glycine co-agonist binding site) or indirectly by activation of GPCRs known to potentiate NMDA receptor function (e.g. the mAChR M₁), has the potential to ameliorate the symptoms of schizophrenia. In both preclinical and in clinical studies, xanomeline, an M₁/M₄ preferring orthosteric agonist has proved efficacious with regard to positive, negative and cognitive symptoms, indicating that M₁ activation is a reasonable approach to the treatment of schizophrenia. More recently, the selective M₁ allosteric agonist TBPB demonstrated efficacy in multiple preclinical models of schizophrenia.

As used herein, the term “schizophrenia or psychosis” includes treatment of those mental disorders as described in DSM-W-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

### 5. Substance-Related Disorders and Addictive Behaviors

The present disclosure provides a method for treating substance-related disorders and addictive behaviors, comprising: administering to a patient in need thereof an effective amount of a compound of the present disclosure. Particular substance-related disorders and addictive behaviors are persisting dementia, persisting amnestic disorder, psychotic disorder or anxiety disorder induced by substance abuse; and tolerance of, dependence on or withdrawal from substances of abuse. The text revision of the fourth edition of the Diagnostic and Statistical Manual of Mental Disorders (DSM-IV-TR) (2000, American Psychiatric Association, Washington DC) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

### 6. Pain

In another aspect, the present disclosure provides a method for treating pain, comprising: administering to a patient in need thereof an effective amount of a compound of the present disclosure. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

### 7. Obesity and Eating Disorders

The present disclosure provides a method for treating obesity or eating disorders associated with excessive food intake and complications associated therewith, comprising: administering to a patient in need thereof an effective amount of a compound of the present disclosure. Obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington DC) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-W-TR. The skilled artisan will recognize that there are alternative nomenclatures, nosologies and classification systems for general medical conditions, and that these systems evolve with medical and scientific progress. Thus, the term “obesity or eating disorders associated with excessive food intake” is intended to include like conditions and disorders that are described in other diagnostic sources.

The compounds are further useful in a method for the prevention, treatment, control, amelioration, or reduction of risk of the diseases, disorders and conditions noted herein. The compounds are further useful in a method for the prevention, treatment, control, amelioration, or reduction of risk of the aforementioned diseases, disorders and conditions in combination with other agents.

The present disclosure is further directed to administration of a selective M₁ receptor modulator for improving treatment outcomes in the context of cognitive or behavioral therapy. That is, in one aspect, the disclosure relates to a cotherapeutic method comprising the step of administering to a mammal an effective amount and dosage of at least one compound of the disclosure in connection with cognitive or behavioral therapy.

In another aspect, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As another example, cognitive or behavioral therapy can be provided before or after administration within a period of time of 1, 2, 3, 4, 5, 6, 7, 8, 9, or 10 half-lives of the administered compound.

b. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

c. Combination Therapies

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present disclosure. When a compound of the present disclosure is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present disclosure.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present disclosure. When a compound of the present disclosure is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present disclosure.

The weight ratio of a disclosed compound to the second active ingredient can be varied and will depend upon the effective dose of each ingredient. Generally, an effective dose of each will be used. Thus, for example, when a compound of the present disclosure is combined with another agent, the weight ratio of a disclosed compound to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

In such combinations disclosed compounds and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In an embodiment, the disclosed compounds can be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies. In another embodiment, the subject compound can be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, buspirone, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, Zolpidem, and salts thereof, and combinations thereof, and the like, or the subject compound can be administered in conjunction with the use of physical methods such as with light therapy or electrical stimulation.

In an embodiment, the disclosed compounds can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In an embodiment, the disclosed compounds can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In an embodiment, the disclosed compounds can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts. thereof.

### 5. Kits

In one aspect, the disclosure provides kits comprising at least one disclosed compound or a pharmaceutically acceptable salt thereof, and one or more of:
(a) at least one agent known to increase mAChR M₁ activity;
(b) at least one agent known to decrease mAChR M₁ activity;
(c) at least one agent known to treat a disorder associated with cholinergic activity;
(d) instructions for treating a disorder associated with cholinergic activity;
(e) instructions for treating a disorder associated with M₁ receptor activity; or
(f) instructions for administering the compound in connection with cognitive or behavioral therapy.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 6. Chemical Synthesis

Compounds of formula (I) or (II) may be prepared by synthetic processes or by metabolic processes. Preparation of the compounds by metabolic processes includes those occurring in the human or animal body (in vivo) or processes occurring in vitro.

Compounds of formula (I) or (II) may be synthesized as shown in the Schemes and Examples set forth below.

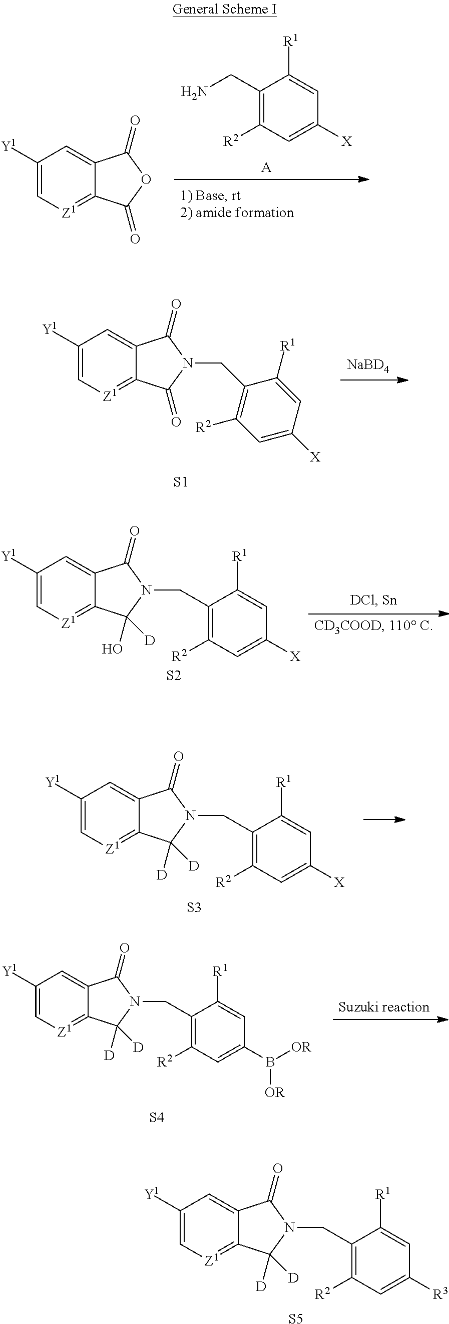

General Scheme I shows a method for preparing compounds of the invention. Reaction of furo[3,4-b]pyridine-5,7-dione and benzyl amine A (e.g., X is halogen, triflate) in the presence of base, followed by treatment with an amide coupling reagent (e.g., HATU) provides intermediate S1. Reaction of intermediates S1 with a reducing agent, such as NaBD₄ may provide intermediate S2. Further reduction of S2 under acidic conditions (e.g., DCl) in the presence of tin powder and deuterated acidic acid-d₄ provide intermediate S3. S3 can be transformed into a boronic acid or ester S4, followed by standard Suzuki conditions to provide analogues S5.

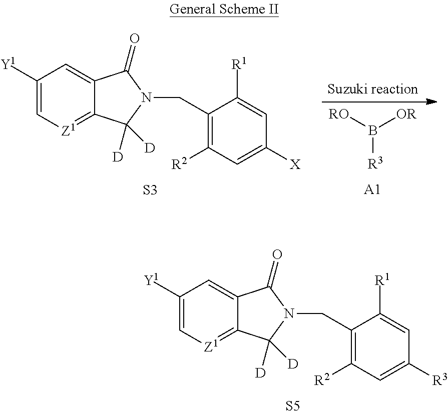

General Scheme II shows an alternative method for preparing compounds of the invention. Reaction of S3 with boronic acids or boronic esters (A1) under standard Suzuki reaction conditions may provide compounds of S5.

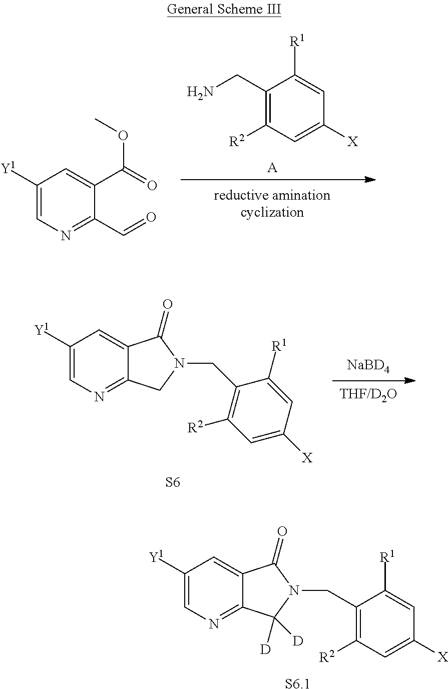

As shown in General Scheme III, intermediates like compound S6 can be prepared via reductive amination and cyclization between 2-formylnicotinate and benzyl amine A (e.g., X is halogen, triflate, boronate ester, R³) with appropriate reducing agents (e.g., Na(OAc)₃BH) to provide intermediates S6. Treatment of S6 with NaOD in THF/D₂O may provide S6.1.

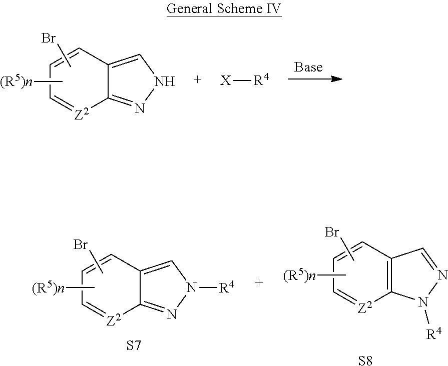

As shown in General Scheme IV, intermediates like compound S7 and S8 can be prepared with base and X—R⁴ (e.g., X is halogen, mesylate, etc.).

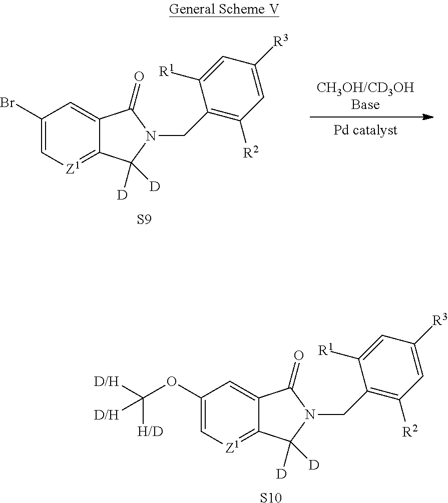

As shown in General Scheme V, Compound S10 may be prepared from intermediate S9 in the presence of CH₃OH or CD₃OH, base (e.g., Cs₂CO₃), and a palladium catalyst (e.g., Rockphos Palladacycle Gen-3 (CAS #2009020-38-4).

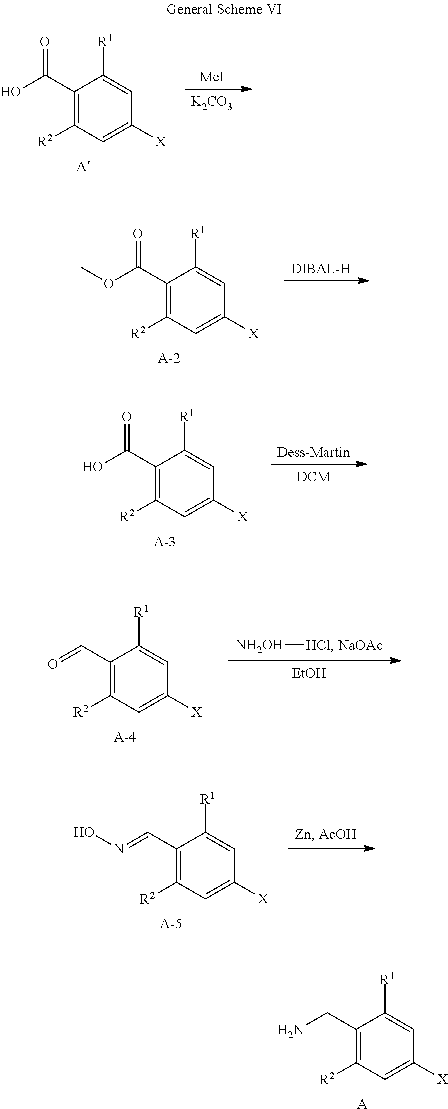

As shown in General Scheme VI, intermediates like benzyl amines of formula A (e.g., X=halogen, R³) may be prepared from benzoic acids of formula A′. Benzoic acids of formula A′ may be subjected to methylation reaction conditions, wherein A′ is reacted with a methylating agent (e.g., Mel) in the presence of a base to form intermediate methyl benzoate of formula A-2. Subsequently, methyl benzoates of formula A-2 may be subjected to reduction conditions, wherein A-2 is reacted with an appropriate reducing agent (e.g., DIBAL-H) to form intermediate benzyl alcohol of formula A-3. Next, benzyl alcohols of formula A-3 may be subjected to oxidation conditions, wherein A-3 is reacted with an appropriate oxidizing agent (e.g., Dess-Martin Periodinane) to form intermediate benzaldehyde of formula A-4. Subsequently, benzaldehydes of formula A-4 may be reacted with a suitable hydroxylamine (e.g., NH₂OH—HCl) to form intermediate oxime of formula A-5. Finally, oximes of formula A-5 may be subjection to reduction conditions, wherein A-5 is reacted with an appropriate reducing agent (e.g., Zn dust/acetic acid) to form benzyl amine intermediates of formula A.

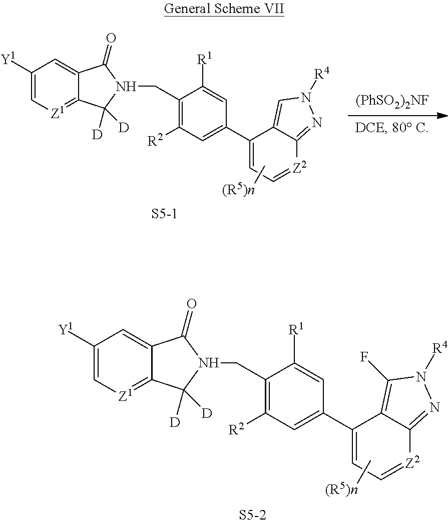

As shown in General Scheme VII, fluorinated compounds of formula S5-2 may be prepared from intermediates of formula S5-1. Intermediates of formula S5-1 may be subjected to fluorination reaction conditions, wherein S5-1 is reacted with a fluorinating agent (e.g., N-fluorobenzenesulfonimide (PhSO₂)₂NF) to form a fluorinated compound of formula S5-2.

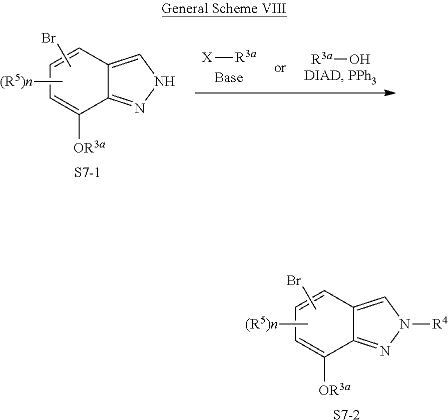

As shown in General Scheme VIII, 7-OR^{3a}-2H-indazoles of formula S7-2 may be prepared from 2H-indazol-7-ols of formula S7-1. 2H-Indazol-7-ols of formula S7-1 may be subjected to alkylation conditions, wherein S7-1 is reacted with an alkyl halide of formula X—R^{3a} in the presence of a base, or, alternatively, S7-1 is reacted with an alcohol of formula R^{3a}—OH under Mitsunobu reaction conditions, to form the 7-OR^{3a}-2H-indazole of formula S7-2.

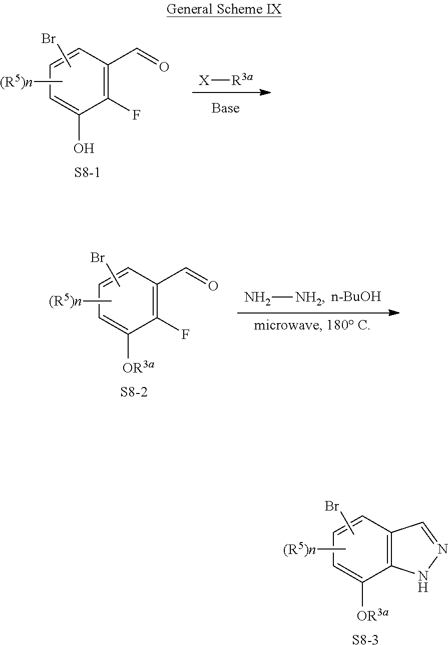

As shown in General Scheme IX, 7-OR^{3a}-1H-indazoles of formula S8-3 may be prepared from 2-fluoro-3-hydroxybenzaldehydes of formula S8-1. 2-Fluoro-3-hydroxybenzaldehydes of formula S8-1 may be subjected to alkylation conditions, wherein S8-1 is reacted with an alkyl halide of formula X—R^{3a} in the presence of a base to form intermediate 2-fluoro-3-OR^{3a}-benzaldehyde of formula S8-2. Subsequently, 2-fluoro-3-OR^{3a}-benzaldehydes of formula S8-2 may be reacted with hydrazine (NH₂—NH₂) under microwave irradiation conditions to form the 7-OR^{3a}-1H-indazole of formula S8-3.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C₁₈ column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C₁₈ column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

The following abbreviations may be used herein:
AcOH acetic acid
Ac₂O acetic anhydride
aq aqueous
atm atmosphere(s)
CDCl₃ chloroform-d
CD₃OD methanol-d₄
CD₃COOD acetic acid-d₄
Celite® diatomaceous earth
conc. concentrated
Cpd compound
CypMgBr cyclopropylmagnesium bromide
DCE dichloroethane
DCl hydrochloric acid-d
DCM dichloromethane
d doublet
dd doublet of doublets
Deoxo-Fluor bis(2-methoxyethyl)amino]sulfur trifluoride
DIPEA/DIEA diisopropylethylamine
DIAD diisopropyl azodicarboxylate
DIBAL-H diisobutylaluminum hydride
DMF N,N-dimethylformamide
DMF•DMA N,N-dimethylformamide dimethyl acetal
DMSO dimethyl sulfoxide
DMSO-d₆ dimethylsulfoxide-d₆ (deuterated dimethylsulfoxide)
D₂O deuterium oxide (deuterated or heavy water)
dppf 1,1′-bis(diphenylphosphino)ferrocene
dppp 1,3-bis(diphenylphosphino)propane
ES-MS electrospray mass spectrometry
EtOAc ethyl acetate
EtOH ethanol
Et₃N triethylamine
eq./equiv equivalents
h or hr hour(s)
HATU 1-[Bis(dimethylamino)methylene]-1H-1,2,3-triazolo[4,5-b]pyridinium 3-oxide hexafluorophosphate
HPLC high performance liquid chromatography
HRMS high resolution mass spectrometry
Hz hertz
iPA isopropyl alcohol
KOAc potassium acetate
LCMS liquid chromatography-mass spectrometry
M molarity (for concentration)
mCPBA 3-chloroperoxybenzoic acid
Me methyl
MeCN acetonitrile
Mel methyl iodide
MeOH methanol
MHz megahertz
min minute(s)
mw microwave
NaHMDS sodium bis(trimethylsilyl)amide
NaOAc sodium acetate
NaOD sodium deuteroxide
NaSMe sodium methanthiolate
Pd(OAc)₂ palladium(II)acetate
Pd₂(dba)₃ Tris(dibenzylideneacetone)dipalladium(O)
Pd(dppf)Cl₂ (1,1′-Bis(diphenylphosphino)ferrocene)palladium(II)
dichloride
Ph phenyl
(PhSO₂)₂NF N-fluorobenzenesulfonimide
ppm parts per million
RockPhos Palladacycle Gen-3 [(2-Di-tert-butylphosphino-3-methoxy-6-methyl-2′,4′,6′-triisopropyl-1,1′-biphenyl)-2-(2-aminobiphenyl)]palladium(II) methanesulfonate
RP reverse phase
r.t./rt/RT room temperature
s singlet
sat. saturated
soln. solution
STAB sodium triacetoxy borohydride
TBTU 2-(1H-Benzotriazole-1-yl)-1,1,3,3-tetramethylaminium tetrafluoroborate
TFA trifluoroacetic acid
TFAA trifluoroacetic anhydride
THE tetrahydrofuran

### Preparation of Intermediates

### Intermediate Example 1. Preparation of benzyl 5H-pyrrolo[3,4-b]pyridine-5,7(6H)-diones

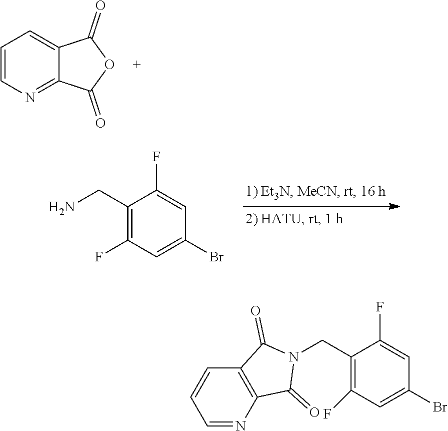 [SMILES: O=C1OC(=O)c2ncccc21 ; NCc1c(F)cc(Br)cc1F ; O=C1c2cccnc2C(=O)N1Cc1c(F)cc(Br)cc1F]

6-(4-Bromo-2,6-difluorobenzyl)-5H-pyrrolo[3,4-b]pyridine-5,7(6H)-dione (Intermediate 1). To a suspension of (4-bromo-2,6-difluorophenyl)methanamine hydrochloride (12.92 g, 50 mmol, 1.0 eq.) in MeCN (500 mL, 0.1 M) was added triethylamine (27.88 mL, 200 mmol, 4.0 eq.). The reaction mixture was stirred at rt for 10 min or until a homogeneous solution was obtained. Furo[3,4-b]pyridine-5,7-dione (7.46 g, 50.0 mmol, 1.0 eq) was added. After stirring for 16 h at rt, HATU (38.02 g, 100 mmol, 2.0 eq.) was added. After 1 h, the mixture was diluted with EtOAc and water and the layers were separated. The organic layer was extracted with EtOAc (3×). The combined extracts were washed with brine, dried over Na₂SO₄, filtered and concentrated. The crude material was purified using normal phase chromatography on silica gel (0-100% EtOAc/hexanes) to provide the title compound (12.83 g, 73%). ¹H NMR (400 MHz, CDCl₃) δ 8.98 (dd, J=4.9, 1.5 Hz, 1H), 8.16 (dd, J=7.6, 1.4 Hz, 1H), 7.62 (dd, J=7.7, 4.9 Hz, 1H), 7.10 (ddd, J=13.3, 6.2, 2.6 Hz, 2H), 4.97 (s, 2H); ES-MS [M+H]⁺=353.2/355.2.

The compounds shown in Table 1 may be prepared similarly to the compound described above, with appropriate starting materials.

TABLE 1

| Structure | Name | ¹H-NMR and/or ES-MS [M + H]⁺ |
|---|---|---|
| 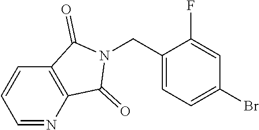 [SMILES: O=C1c2cccnc2C(=O)N1Cc1ccc(Br)cc1F] | 6-(4-Bromo-2-fluoro- benzyl)-5H-pyrrolo- [3,4-b]pyridine- 5,7(6H)-dione | ¹H NMR (400 MHz, CDCl₃) δ 8.99 (dd, J = 5.0, 1.5 Hz, 1H), 8.18 (dd, J = 7.7, 1.5 Hz, 1H), 7.63 (dd, J = 7.7, 5.0 Hz, 1H), 7.23-7.30 (m, 3H), 4.94 (s, 2H); ES-MS [M + H]⁺ = 335.2/337.2. |
| 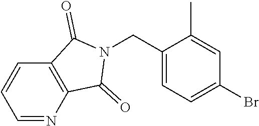 [SMILES: Cc1cc(Br)ccc1CN1C(=O)c2cccnc2C1=O] | 6-(4-Bromo-2-meth- ylbenzyl)-5H- pyrrolo[3,4-b]- pyridine-5,7(6H)- dione | ¹H NMR (400 MHz, CDCl₃) δ 8.99 (dd, J = 5.0, 1.5 Hz, 1H), 8.17 (dd, J = 7.7, 1.5 Hz, 1H), 7.63 (dd, J = 7.6, 5.0 Hz, 1H), 7.36-7.31 (m, 1H), 7.30-7.23 (m, 1H), 7.19 (d, J = 8.2 Hz, 1H), 4.87 (s, 2H), 2.48 (s, 3H); ES-MS [M + H]⁺ = 331.2/333.2. |
| 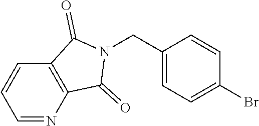 [SMILES: O=C1c2cccnc2C(=O)N1Cc1ccc(Br)cc1] | 6-(4-Bromobenzyl)- 5H-pyrrolo[3,4-b]- pyridine-5,7(6H)- dione | ¹H NMR (400 MHz, CDCl₃) δ 8.97 (dd, J = 4.9, 1.5 Hz, 1H), 8.16 (dd, J = 7.7, 1.5 Hz, 1H), 7.61 (dd, J = 7.6, 4.9 Hz, 1H), 7.49-7.41 (m, 2H), 7.38-7.29 (m, 2H), 4.86 (s, 2H); ES-MS [M + H]⁺ = 317.2/319.2 |
| 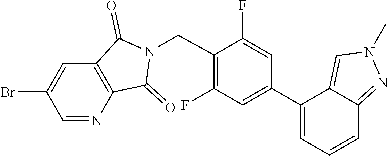 [SMILES: Cn1cc2c(-c3cc(F)c(CN4C(=O)c5cc(Br)cnc5C4=O)c(F)c3)cccc2n1] | 3-Bromo-6-(2,6-difluoro-4-(2-meth- yl-2H-indazol-4- yl)benzyl)-5H- pyrrolo[3,4-b]- pyridine-5,7(6H)- dione | ¹H NMR (400 MHz, DMSO-d₆) δ 9.14 (d, J = 2.1 Hz, 1H), 8.66 (d, J = 2.0 Hz, 1H), 8.61 (d, J = 1.0 Hz, 1H), 7.66 (d, J = 8.5 Hz, 1H), 7.48-7.43 (m, 2H), 7.33 (dd, J = 8.5, 7.0 Hz, 1H), 7.26 (dd, J = 7.0, 0.9 Hz, 1H), 4.93 (s, 2H), 4.19 (s, 3H); ES-MS [M + H]⁺ = 483.0/485.0. |
| 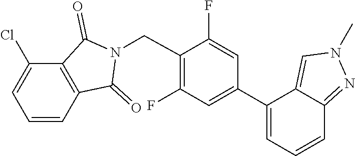 [SMILES: Cn1cc2c(-c3cc(F)c(CN4C(=O)c5cccc(Cl)c5C4=O)c(F)c3)cccc2n1] | 4-chloro-2-(2,6- difluoro-4-(2-meth- yl-2H-indazol-4-yl)- benzyl)isoindoline- 1,3-dione | ES-MS [M + H]⁺ = 438.2 |
| 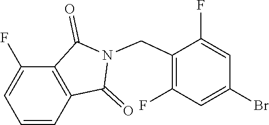 [SMILES: O=C1c2cccc(F)c2C(=O)N1Cc1c(F)cc(Br)cc1F] | 2-(4-bromo-2,6- difluorobenzyl)-4- fluoroisoindoline- 1,3-dione | ES-MS [M + H]⁺ = 370.0/372.0 |
| 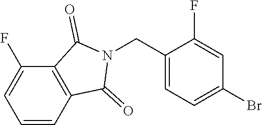 [SMILES: O=C1c2cccc(F)c2C(=O)N1Cc1ccc(Br)cc1F] | 2-(4-bromo-2-fluoro- benzyl)-4-fluoroiso- indoline-1,3-dione | ES-MS [M + H]⁺ = 355.0/357.0. |

### Intermediate Example 2. Preparation of 7-hydroxy-6,7-dihydro-5H-pyrrolo[3,4-b]pyridin-5-ones

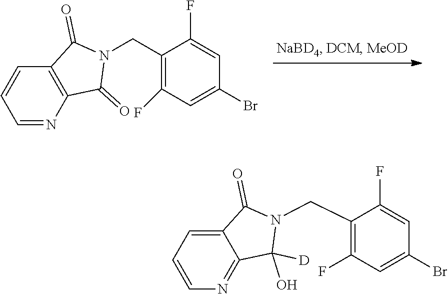 [SMILES: O=C1c2cccnc2C(=O)N1Cc1c(F)cc(Br)cc1F ; [2H]C1(O)c2ncccc2C(=O)N1Cc1c(F)cc(Br)cc1F]

6-(4-Bromo-2,6-difluorobenzyl)-7-hydroxy-6,7-dihydro-5H-pyrrolo[3,4-b]pyridin-5-one-7-d (Intermediate 2) To a solution of 6-(4-bromo-2,6-difluorobenzyl)-5H-pyrrolo[3,4-b]pyridine-5,7(6H)-dione (12.83 g, 36.3 mmol, 1.0 eq.) in DCM (165.15 mL, 0.2 M) was added sodium borodeuteride (1.37 g, 36.3 mmol, 1.0 eq). After 3-5 min at rt, methanol-d₄ (16.15 mL) was added dropwise. After the addition was completed, the reaction mixture was allowed to stir for 30 min and concentrated under reduced pressure with Celite®. Purification using normal phase chromatography on silica gel (0-100% EtOAc/DCM) afforded the title compound as a powder (4.25 g, 33%). ¹H NMR (400 MHz, DMSO-d₆) δ 8.76 (dd, J=5.0, 1.5 Hz, 1H), 8.07 (dd, J=7.6, 1.6 Hz, 1H), 7.55 (dd, J=7.6, 4.9 Hz, 1H), 7.51-7.42 (m, 2H), 6.71 (s, 1H), 4.87 (d, J=14.9 Hz, 1H), 4.54 (d, J=14.9 Hz, 1H); ES-MS [M+H]⁺=356.2/358.2.

The compounds shown in Table 2 may be prepared similarly to the compound described above, with appropriate starting materials.

TABLE 2

| Structure | Name | ¹H-NMR and/or ES-MS [M + H]⁺ |
|---|---|---|
| 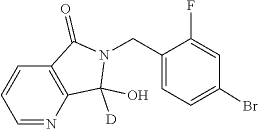 [SMILES: [2H]C1(O)c2ncccc2C(=O)N1Cc1ccc(Br)cc1F] | 6-(4-Bromo-2-fluoro- benzyl)-7-hydroxy- 6,7-dihydro-5H- pyrrolo[3,4-b]pyri- din-5-one-7-d | ¹H NMR (400 MHz, CDCl₃) δ 8.70 (dd, J = 5.1, 1.5 Hz, 1H), 8.16 (dd, J = 7.7, 1.5 Hz, 1H), 7.52 (dd, J = 7.7, 5.1 Hz, 1H), 7.37 (t, J = 8.1 Hz, 1H), 7.28-7.20 (m, 2H), 7.11 (s, 1H), 5.02 (dd, J = 15.1, 1.7 Hz, 1H), 4.70 (dd, J = 15.1, 0.9 Hz, 1H); ES-MS [M + H]⁺ = 338.2/340.2. |
| 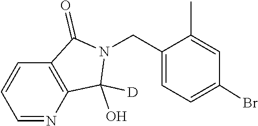 [SMILES: [2H]C1(O)c2ncccc2C(=O)N1Cc1ccc(Br)cc1C] | 6-(4-Bromo-2-methyl- benzyl)-7-hydroxy- 6,7-dihydro-5H- pyrrolo[3,4-b]pyridin- 5-one-7-d | ¹H NMR (400 MHz, CDCl₃) δ 8.62 (dd, J = 5.0, 1.5 Hz, 1H), 8.14 (dd, J = 7.6, 1.5 Hz, 1H), 7.48 (dd, J = 7.7, 5.0 Hz, 1H), 7.34 (d, J = 2.1 Hz, 1H), 7.28 (dd, J = 8.1, 2.1 Hz, 1H), 7.20 (d, J = 8.1 Hz, 1H), 5.60 (s, 1H), 5.16 (d, J = 15.0 Hz, 1H), 4.45 (d, J = 15.0 Hz, 1H), 2.37 (s, 3H); ES-MS [M + H]⁺ = 334.2/336.2. |
| 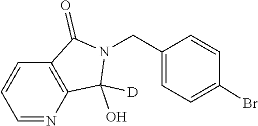 [SMILES: [2H]C1(O)c2ncccc2C(=O)N1Cc1ccc(Br)cc1] | 6-(4-Bromobenzyl)-7- hydroxy-6,7-dihydro- 5H-pyrrolo[3,4-b]pyri- din-5-one-7-d | ¹H NMR (400 MHz, CDCl₃) δ 8.63 (dd, J = 5.1, 1.4 Hz, 1H), 8.13 (dd, J = 7.7, 1.6 Hz, 1H), 7.51-7.39 (m, 3H), 7.30- 7.22 (m, 2H), 5.27 (s, 1H), 5.13 (d, J = 15.0 Hz, 1H), 4.43 (d, J = 14.9 Hz, 1H); ES-MS [M + H]⁺ = 320.3/322.2. |
| 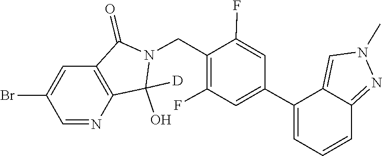 [SMILES: [2H]C1(O)c2ncc(Br)cc2C(=O)N1Cc1c(F)cc(-c2cccc3nn(C)cc23)cc1F] | 3-Bromo-6-(2,6- difluoro-4-(2-methyl- 2H-indazol-4-yl)benz- yl)-7-hydroxy-6,7- dihydro-5H-pyrrolo- [3,4-b]pyridin-5-one- 7-d | ¹H NMR (400 MHz, DMSO-d₆) δ 8.93 (d, J = 2.1 Hz, 1H), 8.62 (s, 1H), 8.37 (d, J = 2.1 Hz, 1H), 7.66 (d, J = 8.5 Hz, 1H), 7.49-7.44 (m, 2H), 7.34 (dd, J = 8.5, 7.0 Hz, 1H), 7.27 (dd, J = 7.0, 0.9 Hz, 1H), 6.87 (s, 1H), 5.02 (d, J = 14.8 Hz, 1H), 4.62 (d, J = 14.9 Hz, 1H), 4.19 (s, 3H); ES-MS [M + H]⁺ = 486.2.3/488.2. |
| 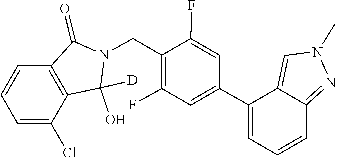 [SMILES: [2H]C1(O)c2c(Cl)cccc2C(=O)N1Cc1c(F)cc(-c2cccc3nn(C)cc23)cc1F] | 4-chloro-2-(2,6- difluoro-4-(2-methyl- 2H-indazol-4-yl)- benzyl)-3-hydroxyiso- indolin-1-one-3-d | ES-MS [M + H]⁺ = 441 |
| 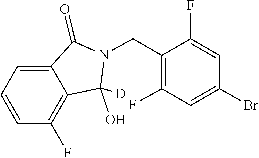 [SMILES: [2H]C1(O)c2c(F)cccc2C(=O)N1Cc1c(F)cc(Br)cc1F] | 2-(4-bromo-2,6- difluorobenzyl)-4- fluoro-3-hydroxyiso- indolin-1-one-3-d | ES-MS [M + H]⁺ = 373.0/375.0 |
| 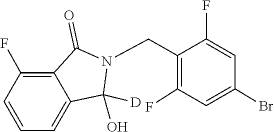 [SMILES: [2H]C1(O)c2cccc(F)c2C(=O)N1Cc1c(F)cc(Br)cc1F] | 2-(4-bromo-2,6- difluorobenzyl)-7- fluoro-3-hydroxyiso- indolin-1-one-3-d | ES-MS [M + H]⁺ = 373.0/375.0 |
| 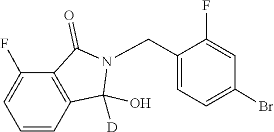 [SMILES: [2H]C1(O)c2cccc(F)c2C(=O)N1Cc1ccc(Br)cc1F] | 2-(4-bromo-2-fluoro- benzyl)-7-fluoro-3- hydroxyisoindolin-1- one-3-d | ES-MS [M + H]⁺ = 355.0/357.0 |
| 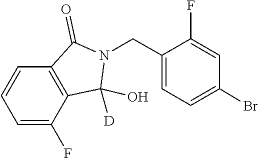 [SMILES: [2H]C1(O)c2c(F)cccc2C(=O)N1Cc1ccc(Br)cc1F] | 2-(4-bromo-2-fluoro- benzyl)-4-fluoro-3- hydroxyisoindolin-1- one-3-d | ES-MS [M + H]⁺ = 355.0/357.0 |

### Intermediate Example 3. Preparation of benzyl 6,7-dihydro-5H-pyrrolo[3,4-b]pyridin-5-ones

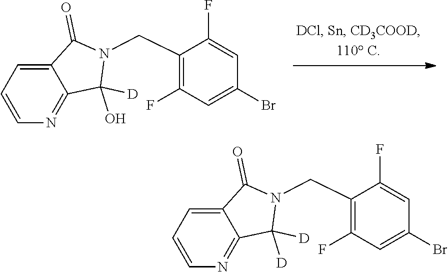 [SMILES: [2H]C1(O)c2ncccc2C(=O)N1Cc1c(F)cc(Br)cc1F ; [2H]C1([2H])c2ncccc2C(=O)N1Cc1c(F)cc(Br)cc1F]

6-(4-Bromo-2,6-difluorobenzyl)-6,7-dihydro-5H-pyrrolo[3,4-b]pyridin-5-one-7,7-d₂ (Intermediate 3). To a suspension of 6-(4-bromo-2,6-difluorobenzyl)-7-hydroxy-6,7-dihydro-5H-pyrrolo[3,4-b]pyridin-5-one-7-d (4.25 g, 11.9 mmol, 1.0 eq.) in acetic acid-d₄ (59.7 mL, 0.2 M) was added tin powder (2.12 g, 17.9 mmol, 1.5 eq) followed by deuterium chloride (DCl) solution (35 wt % in D₂O, 6.39 mL, 59.7 mmol, 5.0 eq). After stirring for 45 min at 110° C., the reaction mixture was concentrated under reduced pressure. The resulting precipitate was re-dissolved in DCM and iPA/CHCl₃ (1:3), adjusted to pH˜7 using a diluted NaHCO₃solution, and the layers were separated. The aqueous layer was extracted with iPA/CHCl₃ (1:3) (5×), and the combined organic layers were washed with brine, dried over Na₂SO₄, filtered, and concentrated. Purification using normal phase chromatography on silica gel (0-100% EtOAc/DCM) provided the title compound (2.34 g, 57% yield). ¹H NMR (400 MHz, CDCl₃) δ 8.73 (dd, J=5.0, 1.6 Hz, 1H), 8.14 (dd, J=7.7, 1.6 Hz, 1H), 7.42 (dd, J=7.7, 4.9 Hz, 1H), 7.22-7.12 (m, 2H), 4.92 (d, J=1.5 Hz, 2H); ES-MS [M+H]⁺=341.1/343.2.

The compounds shown in Table 3 may be prepared similarly to the compound described above, with appropriate starting materials.

TABLE 3

| Structure | Name | ¹H-NMR and/or ES-MS [M + H]⁺ |
|---|---|---|
| 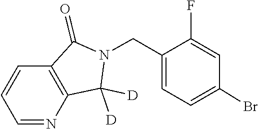 [SMILES: [2H]C1([2H])c2ncccc2C(=O)N1Cc1ccc(Br)cc1F] | 6-(4-Bromo-2-fluoro- benzyl)-6,7-dihydro- 5H-pyrrolo[3,4-b]- pyridin-5-one-7,7-d₂ | ¹H NMR (400 MHz, CDCl₃) δ 8.74 (dd, J = 4.9, 1.6 Hz, 1H), 8.15 (dd, J = 7.7, 1.6 Hz, 1H), 7.43 (dd, J = 7.7, 5.0 Hz, 1H), 7.35-7.24 (m, 3H), 4.86 (d, J = 1.4 Hz, 2H); ES-MS [M + H]⁺ = 323.2/325.2. |
| 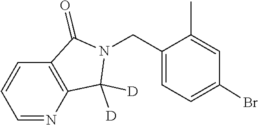 [SMILES: [2H]C1([2H])c2ncccc2C(=O)N1Cc1ccc(Br)cc1C] | 6-(4-Bromo-2-meth- ylbenzyl)-6,7-dihydro- 5H-pyrrolo[3,4-b]- pyridin-5-one-7,7-d₂ | ¹H NMR (400 MHz, CDCl₃) δ 8.74 (dd, J =5.0, 1.6 Hz, 1H), 8.17 (dd, J = 7.7, 1.6 Hz, 1H), 7.44 (dd, J = 7.7, 4.9 Hz, 1H), 7.38 (d, J = 2.1 Hz, 1H), 7.37-7.31 (m, 1H), 7.10 (d, J = 8.1 Hz, 1H), 4.82 (s, 2H), 2.36 (s, 3H); ES-MS [M + H]⁺ = 319.2/321.2. |
| 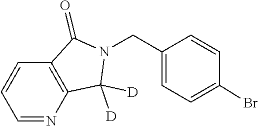 [SMILES: [2H]C1([2H])c2ncccc2C(=O)N1Cc1ccc(Br)cc1] | 6-(4-Bromobenzyl)- 6,7-dihydro-5H- pyrrolo[3,4-b]pyridin- 5-one-7,7-d₂ | ¹H NMR (400 MHz, CDCl₃) δ 8.74 (dd, J =5.0, 1.6 Hz, 1H), 8.17 (dd, J = 7.7, 1.6 Hz, 1H), 7.54-7.47 (m, 2H), 7.43 (dd, J = 7.7, 5.0 Hz, 1H), 7.26-7.18 (m, 2H), 4.80 (s, 2H); ES-MS [M + H]⁺ = 305.2/307.2. |
| 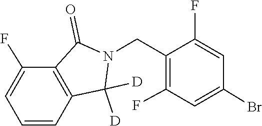 [SMILES: [2H]C1([2H])c2cccc(F)c2C(=O)N1Cc1c(F)cc(Br)cc1F] | 2-(4-bromo-2,6- difluorobenzyl)-7- fluoroisoindolin-1- one-3,3-d₂ | ES-MS [M + H]⁺ = 358.0/360.0 |
| 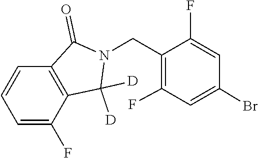 [SMILES: [2H]C1([2H])c2c(F)cccc2C(=O)N1Cc1c(F)cc(Br)cc1F] | 2-(4-bromo-2,6- difluorobenzyl)-4- fluoroisoindolin-1- one-3,3-d₂ | ES-MS [M + H]⁺ = 358.0/360.0 |
| 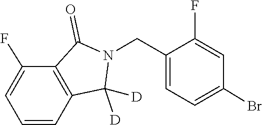 [SMILES: [2H]C1([2H])c2cccc(F)c2C(=O)N1Cc1ccc(Br)cc1F] | 2-(4-bromo-2-fluoro- benzyl)-7-fluoroiso- indolin-1-one-3,3-d₂ | ES-MS [M + H]⁺ = 340.0/342.0 |
| 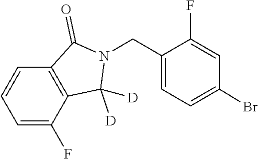 [SMILES: [2H]C1([2H])c2c(F)cccc2C(=O)N1Cc1ccc(Br)cc1F] | 2-(4-bromo-2-fluoro- benzyl)-4-fluoroiso- indolin-1-one-3,3-d₂ | ES-MS [M + H]⁺ = 340.0/342.0 |

### Intermediate Example 4. Preparation of 6,7-dihydro-5H-pyrrolo[3,4-b]pyridin-5-ones Using Reductive Amination

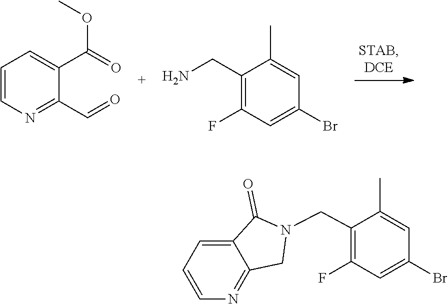 [SMILES: COC(=O)c1cccnc1C=O ; Cc1cc(Br)cc(F)c1CN ; Cc1cc(Br)cc(F)c1CN1Cc2ncccc2C1=O]

Step 1: 6-(4-Bromo-2-fluoro-6-methylbenzyl)-6,7-dihydro-5H-pyrrolo[3,4-b]pyridin-5-one. A solution of methyl 2-formylnicotinate (230 mg, 1.40 mmol) and (4-bromo-2-fluoro-6-methylphenyl)methanamine (319 mg, 1.46 mmol) in DCE (7 mL) was stirred for 10 min. Sodium triacetoxyborohydride (443 mg, 2.10 mmol) was added. After 16 h at rt, the reaction mixture was diluted with DCM and washed with a sat. soln. of NaHCO₃ and brine. The organic layer was dried over Na₂SO₄, filtered, and concentrated. The crude material was purified using normal phase chromatography on silica gel (0-100% EtOAc/DCM) to provide the title compound (450 mg, 96% yield). ¹H NMR (400 MHz, DMSO) δ 8.73 (dd, J=5.0, 1.6 Hz, 1H), 8.10 (dd, J=7.7, 1.6 Hz, 1H), 7.51 (dd, J=7.7, 4.9 Hz, 1H), 7.42 (dd, J=9.4, 2.0 Hz, 1H), 7.35 (s, 1H), 4.78 (d, J=1.9 Hz, 2H), 4.32 (s, 2H), 2.39 (s, 3H). ES-MS [M+H]⁺=335.0/337.0.

The compounds shown in Table 4 may be prepared similarly to the compound described above, with appropriate starting materials.

TABLE 4

| Structure | Name | ¹H-NMR and/or ES-MS [M + H]⁺ |
|---|---|---|
| 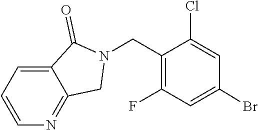 [SMILES: O=C1c2cccnc2CN1Cc1c(F)cc(Br)cc1Cl] | 6-(4-bromo-2-chloro- 6-fluorobenzyl)-6,7- dihydro-5H-pyrrolo- [3,4-b]pyridin-5-one | ¹H NMR (400 MHz, DMSO) δ 8.74 (dd, J = 4.9, 1.6 Hz, 1H), 8.10 (dd, J = 7.7, 1.6 Hz, 1H), 7.72-7.65 (m, 2H), 7.54- 7.49 (m, 1H), 4.88 (d, J = 1.9 Hz, 2H), 4.37 (s, 2H); ES-MS [M + H]⁺ = 355.0/336.9. |
| 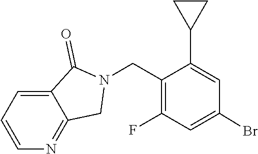 [SMILES: O=C1c2cccnc2CN1Cc1c(F)cc(Br)cc1C1CC1] | 6-(4-bromo-2-cyclo- propyl-6-fluoro- benzyl)-6,7-dihydro- 5H-pyrrolo[3,4-b]- pyridin-5-one | ¹H NMR (400 MHz, CDCl₃) δ 8.70 (dd, J = 5.0, 1.6 Hz, 1H), 8.10 (dd, J = 7.7, 1.6 Hz, 1H), 7.39 (dd, J = 7.7, 4.9 Hz, 1H), 7.14 (dd, J = 9.1, 1.9 Hz, 1H), 6.89 (t, J = 1.4 Hz, 1H), 5.05 (d, J = 2.0 Hz, 2H), 4.29 (s, 2H), 2.17 (tt, J = 8.4, 5.3 Hz, 1H), 1.08-0.96 (m, 2H), 0.69-0.60 (m, 2H); ES-MS [M + H]⁺ = 362.9. |
| 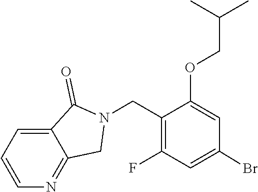 [SMILES: CC(C)COc1cc(Br)cc(F)c1CN1Cc2ncccc2C1=O] | 6-(4-bromo-2-fluoro- 6-isobutoxybenzyl)- 6,7-dihydro-5H- pyrrolo[3,4-b]- pyridin-5-one | ¹H NMR (400 MHz, DMSO) δ 8.72 (dd, J =5.0, 1.6 Hz, 1H), 8.08 (dd, J = 7.7, 1.6 Hz, 1H), 7.50 (dd, J = 7.8, 5.0 Hz, 1H), 7.16 (dd, J = 9.0, 1.7 Hz, 1H), 7.11 (t, J = 1.6 Hz, 1H), 4.76 (d, J = 1.7 Hz, 2H), 4.31 (s, 2H), 3.84 (d, J = 6.3 Hz, 2H), 2.07-1.93 (m, 1H), 0.95 (d, J = 6.7 Hz, 6H).; ES-MS [M + H]⁺ = 393.0/395.0. |
| 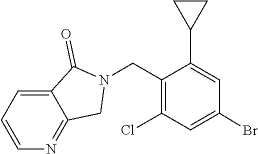 [SMILES: O=C1c2cccnc2CN1Cc1c(Cl)cc(Br)cc1C1CC1] | 6-(4-bromo-2-chloro- 6-cyclopropylbenz- yl)-6,7-dihydro-5H- pyrrolo[3,4-b]pyridin- 5-one | ¹H NMR (400 MHz, CDCl₃) δ 8.70 (dd, J = 5.0, 1.6 Hz, 1H), 8.11 (dd, J = 7.7, 1.6 Hz, 1H), 7.46 (d, J = 2.0 Hz, 1H), 7.39 (dd, J = 7.7, 4.9 Hz, 1H), 7.03 (d, J = 2.0 Hz, 1H), 5.22 (s, 2H), 4.28 (s, 2H), 2.10 (tt, J = 8.4, 5.3 Hz, 1H), 1.08- 0.94 (m, 2H), 0.68-0.60 (m, 2H); ES-MS [M + H]⁺ = 378.9. |
| 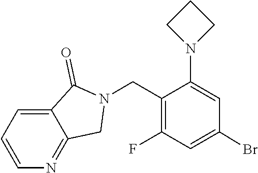 [SMILES: O=C1c2cccnc2CN1Cc1c(F)cc(Br)cc1N1CCC1] | 6-(2-(azetidin-1-yl)- 4-bromo-6-fluoro- benzyl)-6,7-dihydro- 5H-pyrrolo[3,4-b]- pyridin-5-one | ¹H NMR (400 MHz, CDCl₃) δ 8.69 (dd, J = 5.0, 1.6 Hz, 1H), 8.10 (dd, J = 7.7, 1.6 Hz, 1H), 7.37 (dd, J = 7.7, 4.9 Hz, 1H), 6.72 (dd, J = 9.3, 1.9 Hz, 1H), 6.37 (t, J = 1.6 Hz, 1H), 4.80 (d, J = 2.3 Hz, 2H), 4.24 (s, 2H), 4.04 (t, J = 7.3 Hz, 4H), 2.27 (p, J = 7.3 Hz, 2H); ES-MS [M + H]⁺ = 378.0. |
| 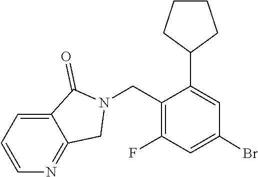 [SMILES: O=C1c2cccnc2CN1Cc1c(F)cc(Br)cc1C1CCCC1] | 6-(4-bromo-2-cyclo- pentyl-6-fluorobenz- yl)-6,7-dihydro-5H- pyrrolo[3,4-b]pyridin- 5-one | ¹H NMR (400 MHz, CDCl₃) δ 8.70 (dd, J = 5.0, 1.6 Hz, 1H), 8.13 (dd, J = 7.7, 1.6 Hz, 1H), 7.40 (dd, J = 7.7, 4.9 Hz, 1H), 7.27-7.25 (m, 1H), 7.15 (dd, J = 9.2, 1.9 Hz, 1H), 4.93 (d, J = 2.3 Hz, 2H), 4.28 (s, 2H), 3.52-3.39 (m, 1H), 2.01-1.89 (m, 2H), 1.84-1.63 (m, 4H), 1.54-1.40 (m, 2H); ES-MS [M + H]⁺ = 391.0. |
| 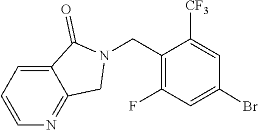 [SMILES: O=C1c2cccnc2CN1Cc1c(F)cc(Br)cc1C(F)(F)F] | 6-(4-bromo-2-fluoro- 6-(trifluoromethyl)- benzyl)-6,7-dihydro- 5H-pyrrolo[3,4-b]- pyridin-5-one | ¹H NMR (400 MHz, DMSO) δ 8.74 (dd, J = 4.9, 1.6 Hz, 1H), 8.12 (dd, J = 7.7, 1.6 Hz, 1H), 8.01 (dd, J = 9.6, 1.9 Hz, 1H), 7.86 (t, J = 1.6 Hz, 1H), 7.52 (dd, J = 7.7, 4.9 Hz, 1H), 4.92 (s, 2H), 4.30 (s, 2H); ES-MS [M + H]⁺ = 388.9/391.0. |
| 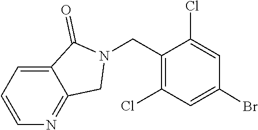 [SMILES: O=C1c2cccnc2CN1Cc1c(Cl)cc(Br)cc1Cl] | 6-(4-bromo-2,6- dichlorobenzyl)-6,7- dihydro-5H-pyrrolo- [3,4-b]pyridin-5-one | ¹H NMR (400 MHz, DMSO) δ 8.74 (dd, J = 4.9, 1.6 Hz, 1H), 8.11 (dd, J = 7.7, 1.6 Hz, 1H), 7.85 (s, 2H), 7.58-7.48 (m, 1H), 4.98 (s, 2H), 4.31 (s, 2H); ES-MS [M + H]⁺ = 372.9/374.9; |
| 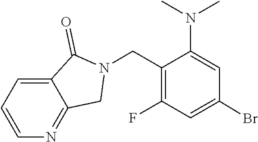 [SMILES: CN(C)c1cc(Br)cc(F)c1CN1Cc2ncccc2C1=O] | 6-(4-bromo-2- (dimethylamino)-6- fluorobenzyl)-6,7- dihydro-5H-pyrrolo- [3,4-b]pyridin-5-one | ¹H NMR (400 MHz, CDCl₃) δ 8.69 (dd, J = 5.0, 1.6 Hz, 1H), 8.14 (dd, J = 7.7, 1.6 Hz, 1H), 7.45-7.37 (m, 1H), 7.07 (t, J =1.6 Hz, 1H), 7.00 (dd, J = 9.1, 1.9 Hz, 1H), 4.99 (d, J = 1.9 Hz, 2H), 4.30 (s, 2H), 275 (s, 6H); ES-MS [M + H]⁺ = 364.0/366.0. |
| 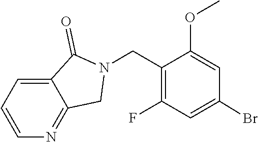 [SMILES: COc1cc(Br)cc(F)c1CN1Cc2ncccc2C1=O] | 6-(4-bromo-2-fluoro- 6-methoxybenzyl)- 6,7-dihydro-5H- pyrrolo[3,4-b]pyridin- 5-one | ¹H NMR (400 MHz, DMSO) δ 8.72 (dd, J = 4.9, 1.6 Hz, 1H), 8.07 (dd, J = 7.7, 1.6 Hz, 1H), 7.50 (dd, J = 7.7, 4.9 Hz, 1H), 7.20-7.11 (m, 2H), 4.72 (d, J = 1.7 Hz, 2H), 4.35 (s, 2H), 3.88 (s, 3H); ES-MS [M + H]⁺ = 351.0/353.0. |
| 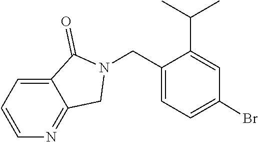 [SMILES: CC(C)c1cc(Br)ccc1CN1Cc2ncccc2C1=O] | 6-(4-bromo-2-iso- propylbenzyl)-6,7- dihydro-5H-pyrrolo- [3,4-b]pyridin-5-one | ¹H NMR (400 MHz, CDCl₃) δ 8.71 (dd, J = 5.0, 1.6 Hz, 1H), 8.16 (dd, J = 7.7, 1.6 Hz, 1H), 7.47-7.39 (m, 2H), 7.31 (dd, J = 8.2, 2.1 Hz, 1H), 7.10 (d, J = 8.2 Hz, 1H), 4.85 (s, 2H), 4.26 (s, 2H), 3.26 (hept, J = 6.8 Hz, 1H), 1.18 (d, J = 6.8 Hz, 6H); ES-MS [M + H]⁺ = 346.9. |

### Intermediate Example 5: (4-Bromo-2-chloro-6-fluorophenyl)methanamine

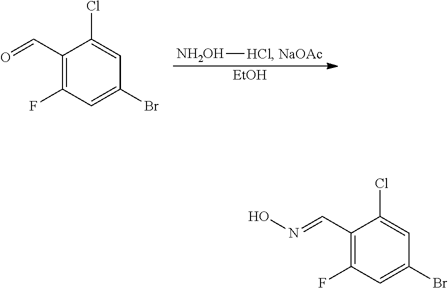

Step 1: (E/Z)-4-Bromo-2-chloro-6-fluorobenzaldehyde oxime. To a suspension of 4-bromo-2-chloro-6-fluorobenzaldehyde (3.56 g, 15 mmol) in ethanol (75 mL) was added sodium acetate (2.46 g, 30 mmol) and hydroxylamine hydrochloride (2.08 g, 30 mmol). After 1 h at rt, the reaction mixture was diluted with EtOAc, then washed with H₂O and brine. The organic layer was dried over Na₂SO₄, filtered, and concentrated to provide the title compound, which was carried forward without further purification (3.75 g). ¹H NMR (400 MHz, DMSO-d₆) δ 8.17 (s, 1H), 7.75-7.69 (m, 2H), OH proton exchangeable; ES-MS [M+H]⁺=251.9/253.9.

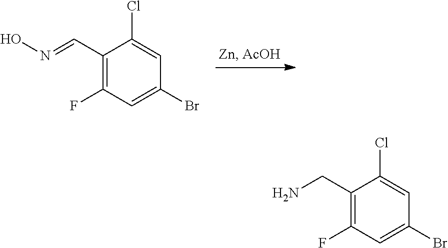 [SMILES: O/N=C/c1c(F)cc(Br)cc1Cl ; NCc1c(F)cc(Br)cc1Cl]

Step 2: (4-Bromo-2-chloro-6-fluorophenyl)methanamine. To (E/Z)-4-bromo-2-chloro-6-fluorobenzaldehyde oxime (3.75 g, 15 mmol) in acetic acid (74 mL) was added zinc powder (4.85 g, 74.3 mmol). The resulting suspension was sonicated at rt for 3 h then filtered through a pad of Celite®, which was washed thoroughly with 10% MeOH/DCM. The filtrate was concentrated under reduced pressure, azeotroped with toluene (3×), and then dried under vacuum to provide the title compound as an acetate salt. Quantative yield was assumed, and the product was carried forward without further purification. ¹H NMR (400 MHz, DMSO-d₆) δ 7.65-7.63 (m, 1H), 7.62 (dd, J=9.0, 1.9 Hz, 1H), 3.87 (d, J=1.8 Hz, 2H), NH protons exchangeable; ES-MS [M+H]⁺=237.9/240.2.

The compounds shown in Table 5 may be prepared similarly to the compound described above, with appropriate starting materials.

TABLE 5

| | | ¹H-NMR and/or ES-MS |
|---|---|---|
| Structure | Name | [M + H]⁺ |
| 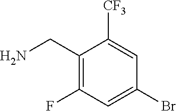 [SMILES: NCc1c(F)cc(Br)cc1C(F)(F)F] | (4-bromo-2-fluoro-6- (trifluoromethyl)phenyl)methanamine | ¹H NMR (400 MHz, DMSO) δ 7.99 (dd, J = 9.4, 1.9 Hz, 1H), 7.77 (t, J = 1.5 Hz, 1H), 3.86 (s, 2H), NH protons exchangeable; ES-MS [M + H]⁺ = 271.9/273.8. |
| 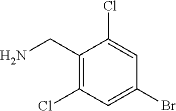 [SMILES: NCc1c(Cl)cc(Br)cc1Cl] | (4-bromo-2,6- dichlorophenyl)methanamine | ¹H NMR (400 MHz, DMSO) δ 7.78 (s, 2H), 3.95 (s, 2H), NH protons exchangeable; ES-MS [M + H]⁺ = 255.8/257.8. |
| 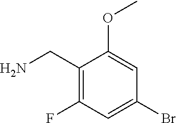 | (4-bromo-2-fluoro-6- methoxyphenyl)methanamine | ¹H NMR (400 MHz, CDCl₃) δ 6.90 (dd, J = 8.6, 1.7 Hz, 1H), 6.83 (d, J = 1.5 Hz, 1H), 4.00 (s, 2H), 3.87 (s, 3H), 3.70 (s, 2H); ES-MS [M + H]⁺ = 239.9/241.9. |
| 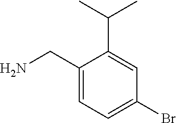 | (4-bromo-2- isopropylphenyl)methanamine | ES-MS [M + H]⁺ = 228.0/230.0. |

### Intermediate Example 6: (4-bromo-2-cyclopropyl-6-fluorophenyl)methanamine

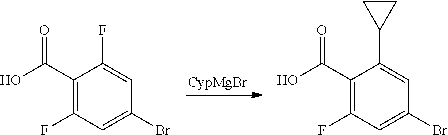 [SMILES: O=C(O)c1c(F)cc(Br)cc1F ; O=C(O)c1c(F)cc(Br)cc1C1CC1]

Step 1: 4-Bromo-2-cyclopropyl-6-fluorobenzoic acid. To an oven-dried 250 mL flask was added 4-bromo-2,6-difluoro-benzoic acid (5.0 g, 21.1 mmol) and THE (100 mL) under N2. The reaction was cooled to 0° C. Cyclopropylmagnesium bromide (75.35 mL, 52.74 mmol,) was added slowly and the reaction was slowly warm to rt over 1 h. After 16 h, the reaction was cooled to 0° C., recharged with cyclopropylmagnesium bromide (15.07 mL, 10.55 mmol, ˜ 0.7 mol/L, 0.5 eq), and slowly warmed to rt. This step was repeated after another 16 h. Upon completion, the reaction was carefully quenched with water at 0° C. and concentrated under reduced pressure. The residue was acidified by 1M HCl (aq.). The aqueous phase was extracted with EtOAc (3×). The combined extracts were washed with water, brine, dried (Na₂SO₄), filtered and concentrated to give the crude product (5.46 g, 99% yield) as a white solid. The crude product was used in the next step directly without further purification. ES-MS [M+H]⁺=260.9.

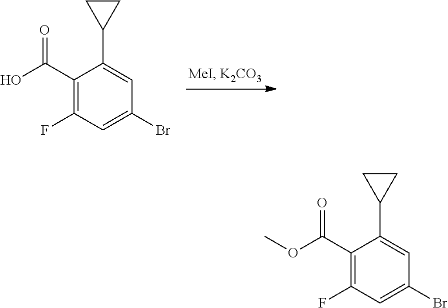

Step 2: Methyl 4-bromo-2-cyclopropyl-6-fluorobenzoate: To a solution of 4-bromo-2-cyclopropyl-6-fluoro-benzoic acid (5.46 g, 21.08 mmol) in DMF (42 mL) was added potassium carbonate (8.87 g, 63.23 mmol) at rt. Mel (2.62 mL, 42.15 mmol) was added slowly. After 16 h, the reaction mixture was quenched with water and extracted with EtOAc (3×). The combined extracts were washed with brine, dried (Na₂SO₄), filtered, and concentrated. The crude was purified using normal phase chromatography on silica gel (0-10% EtOAc/hexanes) to give the title compound (4.51 g, 65% yield). ES-MS [M+H]⁺=274.8.

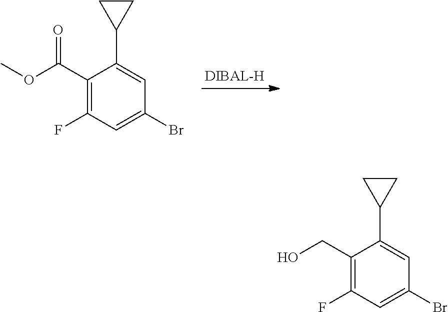

Step 3: (4-Bromo-2-cyclopropyl-6-fluorophenyl)methanol. To a solution of methyl 4-bromo-2-cyclopropyl-6-fluoro-benzoate (4.51 g, 16.51 mmol) in DCM (80 mL) was added diisobutylaluminum hydride solution (1.0 M in THF, 49.54 mL, 49.54 mmol) slowly at 0° C. The reaction was slowly warmed to rt over 2 h. After 16 h, the reaction was cooled to 0° C. and diisobutylaluminum hydride solution (16.51 mL, 16.51 mmol, 1.0 M in THF, 1.0 eq) was added, After 5 h at 0° C., the reaction was carefully quenched with sat. aq. NH₄Cl dropwise. The residue was filtered through Celite and washed with EtOAc. The filtrate was concentrated in vacuo. The crude residue was dissolved in DCM (20 mL), filtered through a phase separator, and then concentrated to give the title compound (3.96 g, 98% yield). ¹H NMR (400 MHz, CDCl₃) δ 7.09 (dd, J=9.1, 1.9 Hz, 1H), 6.93 (dt, J=1.8, 0.8 Hz, 1H), 4.88 (d, J=1.9 Hz, 2H), 2.15 (tt, J=8.4, 5.3 Hz, 1H), 1.10-0.97 (m, 2H), 0.76-0.68 (m, 2H); ES-MS [M−H₂O+H]⁺=228.9.

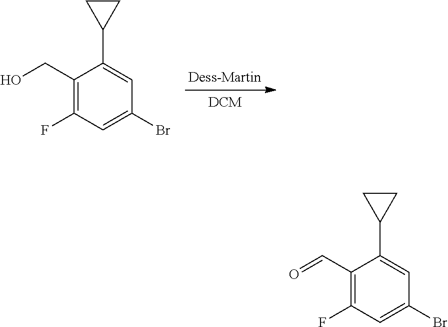 [SMILES: OCc1c(F)cc(Br)cc1C1CC1 ; O=Cc1c(F)cc(Br)cc1C1CC1]

Step 4: 4-Bromo-2-cyclopropyl-6-fluorobenzaldehyde. To a solution of (4-bromo-2-cyclopropyl-6-fluoro-phenyl)methanol (1.74 g, 7.10 mmol) in DCM (40 mL) was added Dess-martin periodinane (6.02 g, 14.20 mmol) at rt. After 2 h, the reaction was quenched by sat. aq. NaHCO₃. The residue was extracted with DCM (3×). The combined organic layers were dried (Na₂SO₄), filtered, and concentrated in vacuo to give the crude material. The crude material was purified using silica gel chromatography (0-20% EtOAc/hexanes) to give the title compound (1.66 g, 96% yield). ¹H NMR (400 MHz, CDCl₃) δ 10.55 (s, 1H), 7.17 (dd, J=10.2, 1.8 Hz, 1H), 6.97 (dd, J=1.8, 1.0 Hz, 1H), 2.86 (tt, J=8.5, 5.4 Hz, 1H), 1.17-1.05 (m, 2H), 0.78-0.70 (m, 2H); MS [M+H]⁺=245.0.

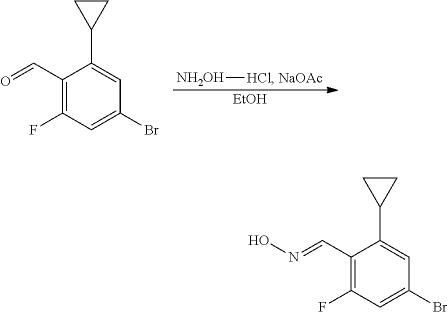

Step 5: (E/Z)-4-Bromo-2-cyclopropyl-6-fluorobenzaldehyde oxime was prepared in a similar manner as (E/Z)-4-bromo-2-chloro-6-fluorobenzaldehyde oxime. ES-MS [M+H]⁺=259.9.

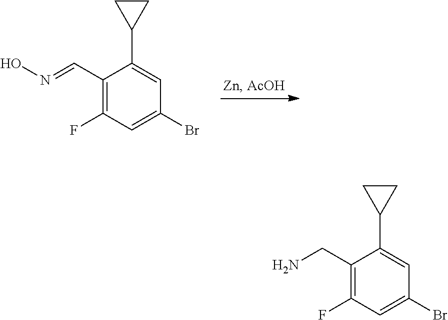 [SMILES: O/N=C/c1c(F)cc(Br)cc1C1CC1 ; NCc1c(F)cc(Br)cc1C1CC1]

Step 6: (4-Bromo-2-cyclopropyl-6-fluorophenyl)methanamine was prepared in a similar manner as (4-bromo-2-chloro-6-fluorophenyl)methanamine. ES-MS [M+H]⁺=246.0.

The compounds shown in Table 6 may be prepared similarly to the compound described above, with appropriate starting materials.

TABLE 6

| | | ¹H-NMR and/or ES-MS |
|---|---|---|
| Structure | Name | [M + H]⁺ |
| 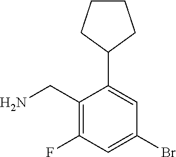 [SMILES: NCc1c(F)cc(Br)cc1C1CCCC1] | (4-bromo-2-cyclopentyl-6- fluorophenyl)methanamine | ES-MS [M + H]⁺ = 271.9/273.9. |
| 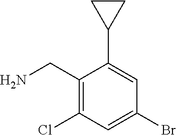 [SMILES: NCc1c(Cl)cc(Br)cc1C1CC1] | (4-bromo-2-chloro-6- cyclopropylphenyl)methanamine | ES-MS [M + H]⁺ = 260.0. |

### Intermediate Example 7: (4-Bromo-2-fluoro-6-isobutoxyphenyl)methanamine

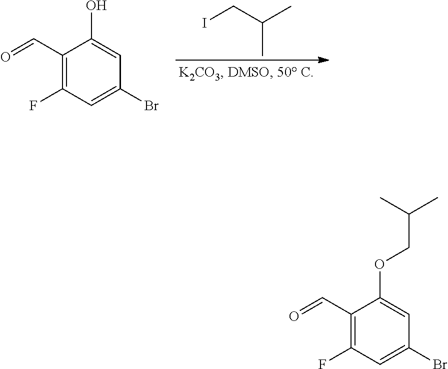

Step 1: 4-Bromo-2-fluoro-6-isobutoxybenzaldehyde. To a suspension of 6-fluoro-4-bromosalicylaldehyde (500 mg, 2.28 mmol) in MeCN (22.8 ml) was added K₂CO₃ (640 mg, 4.57 mmol) followed by 1-iodo-2-methylpropane (0.53 ml, 4.57 mmol). The mixture was stirred at 50° C. for 72 h and recharged with 1-iodo-2-methylpropane (0.53 ml, 4.57 mmol) every 24 h. Upon completion, water was added, and the resulting solution was extracted with EtOAc (3×). The combined extracts were washed with water, brine, dried over Na₂SO₄, filtered, and concentrated. The crude material was purified using normal phase chromatography on silica gel (0-30% EtOAc/hexanes) to provide the title compound as a yellow oil (500 mg, 80% yield). ES-MS [M+H]⁺=277.9/276.9.

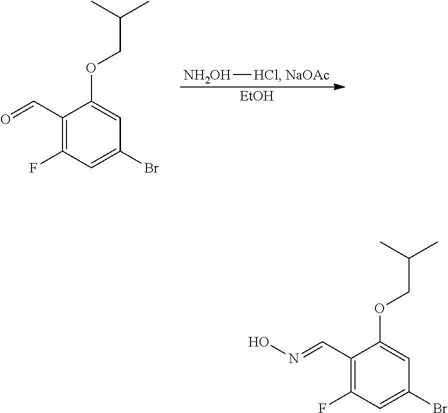

Step 2: (E/Z)-4-Bromo-2-fluoro-6-isobutoxybenzaldehyde oxime was prepared in a similar manner as (E/Z)-4-bromo-2-chloro-6-fluorobenzaldehyde oxime. ES-MS [M+H]⁺=291.

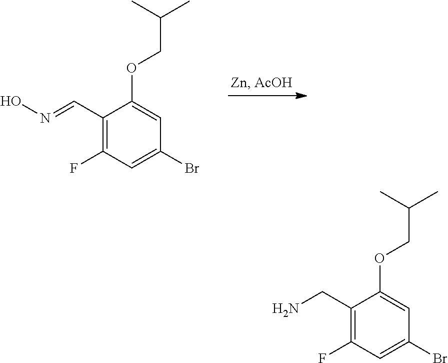 [SMILES: CC(C)COc1cc(Br)cc(F)c1/C=N/O ; CC(C)COc1cc(Br)cc(F)c1CN]

Step 3: (4-Bromo-2-fluoro-6-isobutoxyphenyl)methanamine was prepared in a similar manner as (4-bromo-2-chloro-6-fluorophenyl)methanamine. ¹H NMR (400 MHz, DMSO) δ 7.10 (dd, J=8.9, 1.8 Hz, 1H), 7.06 (t, J=1.5 Hz, 1H), 3.83 (d, J=6.4 Hz, 2H), 3.73 (s, 2H), 2.14-2.00 (m, 1H), 0.99 (d, J=6.7 Hz, 6H); ES-MS [M+H]⁺=277.9.

### Intermediate Example 8: 2-(Aminomethyl)-5-bromo-3-fluoro-N,N-dimethylaniline

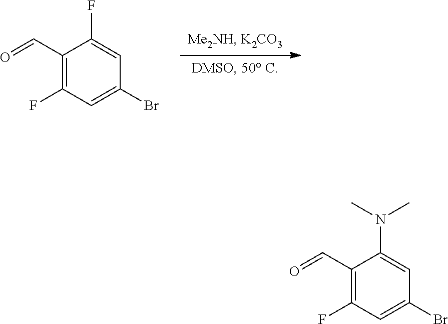

Step 1: 4-Bromo-2-(dimethylamino)-6-fluorobenzaldehyde. To a suspension of 4-bromo-2,6-difluorobenzaldehyde (1.33 g, 6.0 mmol) in DMSO (8 mL) was added K₂CO₃ (2.02 g, 14.4 mmol) followed by dimethylamine hydrochloride (587 mg, 7.2 mmol). The mixture was stirred at 50° C. for 1 h. After cooling to rt, the reaction mixture was poured into ice cold water and extracted with EtOAc (3×). The combined extracts were washed with brine, dried over Na₂SO₄, filtered, and concentrated. The crude material was purified using normal phase chromatography on silica gel (0-30% EtOAc/hexanes) to provide the title compound (1.41 g, 95% yield). ¹H NMR (400 MHz, CDCl₃) δ 10.19 (d, J=0.6 Hz, 1H), 6.92 (t, J=1.5 Hz, 1H), 6.76 (dd, J=10.4, 1.7 Hz, 1H), 2.93 (s, 6H); ES-MS [M+H]⁺=246.0/247.9

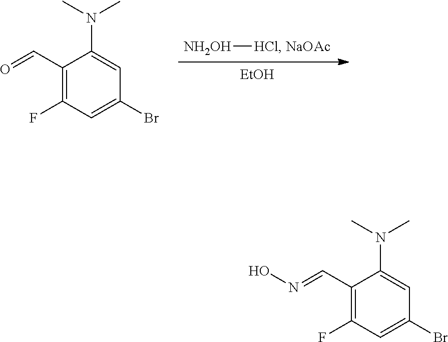

Step 2: (E/Z)-4-Bromo-2-(dimethylamino)-6-fluorobenzaldehyde oxime was prepared in a similar manner as (E/Z)-4-bromo-2-chloro-6-fluorobenzaldehyde oxime. ¹H NMR (400 MHz, CDCl₃) δ 8.20 (s, 1H), 6.97 (d, J=8.5 Hz, 2H), 2.81 (s, 6H), OH proton exchangeable; ES-MS [M+H]⁺=261.0/262.9.

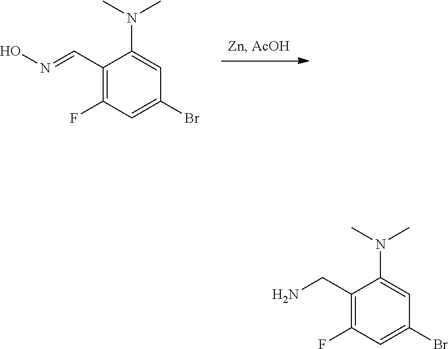

Step 3: 2-(Aminomethyl)-5-bromo-3-fluoro-N,N-dimethylaniline was prepared in a similar manner as (4-bromo-2-chloro-6-fluorophenyl)methanamine. ¹H NMR (400 MHz, CDCl₃) δ 7.06 (t, J=1.6 Hz, 1H), 7.00 (dd, J=9.0, 1.8 Hz, 1H), 6.83 (s, 2H), 4.14 (d, J=1.1 Hz, 2H), 2.70 (s, 6H); ES-MS [M+H]⁺=245.0/247.0.

The compound shown in Table 7 may be prepared similarly to the compound described above, with appropriate starting materials.

TABLE 7

| Structure | Name | ¹H-NMR and/or ES-MS [M + H]⁺ |
|---|---|---|
| 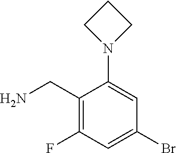 [SMILES: NCc1c(F)cc(Br)cc1N1CCC1] | (2-(azetidin-1-yl)-4- bromo-6- fluorophenyl)methanamine | ES-MS [M − NH₃]⁺ = 241.9/243.9. |

### Intermediate Example 9: Preparation of 6-(4-bromo-2-fluoro-6-methylbenzyl)-6,7-dihydro-5H-pyrrolo[3,4-b]pyridin-5-one-7,7-d₂

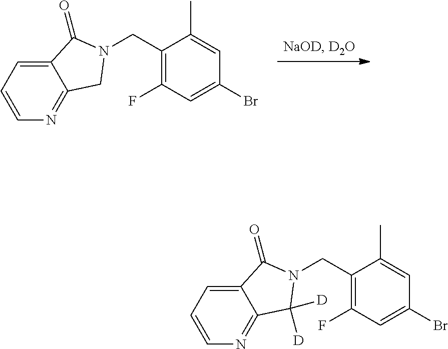

6-(4-Bromo-2-fluoro-6-methylbenzyl)-6,7-dihydro-5H-pyrrolo[3,4-b]pyridin-5-one-7,7-d₂. To a solution of 6-(4-bromo-2-fluoro-6-methylbenzyl)-6,7-dihydro-5H-pyrrolo[3,4-b]pyridin-5-one (335 mg, 0.7 mmol) in THE (5.0 mL) and deuterium oxide (2.28 mL) was added sodium deuteroxide (40% w/w solution in D₂O, 47 μL, 0.7 mmol). The resulting mixture was stirred at 35° C. After 16 h, the mixture was diluted with EtOAc. The layers were separated. The aqueous layer was extracted with EtOAc (3×). The combined extracts were washed with brine, dried (Na₂SO₄), filtered, and concentrated. The crude material was purified using normal phase chromatography on silica gel (0-100% EtOAc/hexanes) to provide the title compound (184 mg, 70% yield). ¹H NMR (400 MHz, DMSO) δ 8.73 (dd, J=4.9, 1.6 Hz, 1H), 8.10 (dd, J=7.7, 1.6 Hz, 1H), 7.51 (dd, J=7.7, 4.9 Hz, 1H), 7.42 (dd, J=9.5, 2.0 Hz, 1H), 7.35-7.33 (m, 1H), 4.77 (d, J=1.9 Hz, 2H), 2.39 (s, 3H); ES-MS [M+H]⁺=337/339.

The compounds shown in Table 5 may be prepared similarly to the compound described above, with appropriate starting materials.

TABLE 8

| Structure | Name | ¹H-NMR and/or ES-MS [M + H]⁺ |
|---|---|---|
| 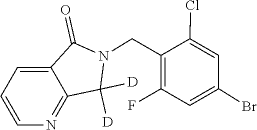 [SMILES: [2H]C1([2H])c2ncccc2C(=O)N1Cc1c(F)cc(Br)cc1Cl] | 6-(4-bromo-2-chloro-6- fluorobenzyl)-6,7- dihydro-5H-pyrrolo[3,4- b]pyridin-5-one-7,7-d₂ | ¹H NMR (400 MHz, DMSO) δ 8.74 (dd, J = 4.9, 1.6 Hz, H), 8.10 (dd, J = 7.7, 1.6 Hz, 1H), 7.71-7.65 (m, 2H), 7.52 (dd, J = 7.7, 5.0 Hz, 1H), 4.87 (d, J = 1.9 Hz, 2H). ES-MS [M + H]⁺ = 357/359 |
| 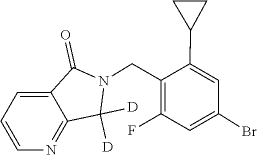 [SMILES: [2H]C1([2H])c2ncccc2C(=O)N1Cc1c(F)cc(Br)cc1C1CC1] | 6-(4-bromo-2- cyclopropyl-6- fluorobenzyl)-6,7- dihydro-5H-pyrrolo[3,4- b]pyridin-5-one-7,7-d₂ | ¹H NMR (400 MHz, CDCl₃) δ 8.67 (d, J = 5.0 Hz, 1H), 8.07 (dd, J = 7.7, 1.5 Hz, 1H), 7.36 (dd, J = 7.7, 4.9 Hz, 1H), 7.11 (dd, J = 9.1, 1.9 Hz, 1H), 6.89-6.82 (m, 1H), 5.02 (d, J = 2.1 Hz, 2H), 2.15 (tt, J = 8.5, 5.3 Hz, 1H), 1.06-0.93 (m, 2H), 0.68-0.58 (m, 2H); ES-MS [M + H]⁺ = 364.9. |
| 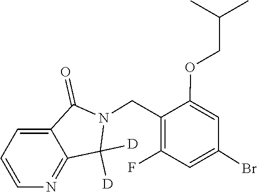 [SMILES: [2H]C1([2H])c2ncccc2C(=O)N1Cc1c(F)cc(Br)cc1OCC(C)C] | 6-(4-bromo-2-fluoro-6- isobutoxybenzyl)-6,7- dihydro-5H-pyrrolo[3,4- b]pyridin-5-one-7,7-d₂ | ¹H NMR (400 MHz, DMSO) δ 8.71 (dd, J = 5.9, 1.6 Hz, 1H), 8.08 (dd, J = 7.7, 1.6 Hz, 1H), 7.50 (dd, J = 7.7, 4.9 Hz, 1H), 7.16 (dd, J = 9.0, 1.7 Hz, 1H), 7.10 (t, J = 1.5 Hz, 1H), 4.76 (d, J = 1.6 Hz, 2H), 3.84 (d, J = 6.3 Hz, 2H), 2.07-1.93 (m, 1H), 0.95 (d, J = 6.7 Hz, 6H).; ES-MS [M + H]⁺ = 395.1/397.0. |
| 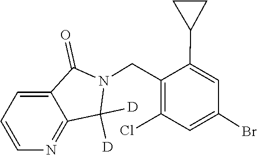 [SMILES: [2H]C1([2H])c2ncccc2C(=O)N1Cc1c(Cl)cc(Br)cc1C1CC1] | 6-(4-bromo-2-chloro-6- cyclopropylbenzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.11 (dd, J = 7.7, 1.6 Hz, 1H), 7.46 (d, J = 2.0 Hz, 1H), 7.39 (dd, J = 7.7, 4.9 Hz, 1H), 7.03 (d, J = 2.0 Hz, 1H), 5.21 (s, 2H), 2.10 (tt, J = 8.4, 5.3 Hz, 1H), 1.06-0.97 (m, 2H), 0.68-0.58 (m, 2H); ES-MS [M + H]⁺ = 381.1. |
| 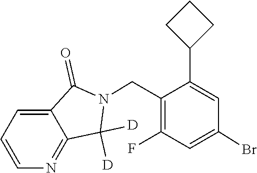 [SMILES: [2H]C1([2H])c2ncccc2C(=O)N1Cc1c(F)cc(Br)cc1C1CCC1] | 6-(2-azetidin-1-yl)-4- bromo-6-fluorobenzyl)- 6,7-dihydro-5H- pyrrolo[3,4-b]pyridin-5- one-7,7-d₂ | ¹H NMR (400 MHz, CDCl₃) δ 8.70 (dd, J = 5.0, 1.6 Hz, 1H), 8.10 (dd, J = 7.7, 1.6 Hz, 1H), 7.38 (dd, J = 7.7, 4.9 Hz, 1H), 6.73 (dd, J = 9.4, 1.9 Hz, 1H), 6.38 (t, J = 1.6 Hz, 1H), 4.81 (d, J = 2.3 Hz, 2H), 4.05 (t, J = 7.3 Hz, 4H), 2.28 (p, J = 7.3 Hz, 2H); ES-MS [M + H]⁺ = 380.0. |
| 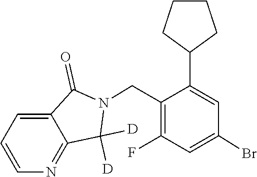 [SMILES: [2H]C1([2H])c2ncccc2C(=O)N1Cc1c(F)cc(Br)cc1C1CCCC1] | 6-(4-bromo-2- cyclopentyl-6- fluorobenzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.12 (dd, J = 7.7, 1.6 Hz, 1H), 7.39 (dd, J = 7.7, 4.9 Hz, 1H), 7.27-7.25 (m, 1H), 7.15 (dd, J = 9.2, 1.9 Hz, 1H), 4.93 (d, J = 2.3 Hz, 2H), 3.53- 3.40 (m, 1H), 2.00-1.88 (m, 2H), 1.85- 1.64 (m, 4H), 1.55-1.41 (m, 2H); ES-MS [M + H]⁺ = 393.0. |
| 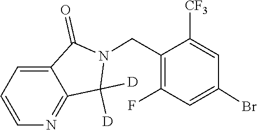 [SMILES: [2H]C1([2H])c2ncccc2C(=O)N1Cc1c(F)cc(Br)cc1C(F)(F)F] | 6-(4-bromo-2-fluoro-6- (trifluoromethyl)benzyl)- 6,7-dihydro-5H- pyrrolo[3,4-b]pyridin-5- one-7,7-d₂ | ¹H NMR (400 MHz, DMSO) δ 8.74 (dd, J = 5.0, 1.6 Hz, 1H), 8.12 (dd, J = 7.7, 1.6 Hz, 1H), 8.01 (dd, J = 9.6, 1.9 Hz, 1H), 7.86 (d, J = 1.7 Hz, 1H), 7.52 (dd, J = 7.7, 4.9 Hz, 1H), 4.92 (s, 2H); ES-MS [M + H]⁺ = 391.0/393.0. |
| 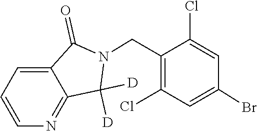 [SMILES: [2H]C1([2H])c2ncccc2C(=O)N1Cc1c(Cl)cc(Br)cc1Cl] | 6-(4-bromo-2,6- dichlorobenzyl)-6,7- dihydro-5H-pyrrolo[3,4- b]pyridin-5-one-7,7-d₂ | ¹H NMR (400 MHz, DMSO) δ 8.74 (dd, J = 5.0, 1.7 Hz, 1H), 8.11 (dd, J = 7.7, 1.6 Hz, 1H), 7.85 (s, 2H), 7.54-7.50 (m, 1H), 4.98 (s, 2H); ES-MS [M + H]⁺ = 374.9/376.9. |
| 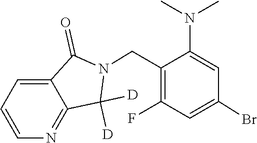 [SMILES: [2H]C1([2H])c2ncccc2C(=O)N1Cc1c(F)cc(Br)cc1N(C)C] | 6-(4-bromo-2- (dimethylamino)-6- fluorobenzyl)-6,7- dihydro-5H-pyrrolo[3,4- b]pyridin-5-one-7,7-d₂ | ¹H NMR (400 MHz, DMSO) δ 8.73 (dd, J = 4.9, 1.6 Hz, 1H), 8.09 (dd, J = 7.7, 1.6 Hz, 1H), 7.51 (dd, J = 7.7, 4.9 Hz, 1H), 7.21 (dd, J = 9.5, 1.9 Hz, 1H), 7.16 (t, J = 1.5 Hz, 1H), 4.84 (d, J = 1.7 Hz, 2H); ES- MS [M + H]⁺ = 366.0/368.0. |
| 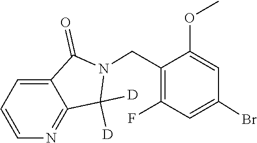 [SMILES: [2H]C1([2H])c2ncccc2C(=O)N1Cc1c(F)cc(Br)cc1OC] | 6-(4-bromo-2-fluoro-6- methoxybenzyl)-6,7- dihydro-5H-pyrrolo[3,4- b]pyridin-5-one-7,7-d₂ | ¹H NMR (400 MHz, DMSO) δ 8.72 (dd, J = 4.9, 1.6 Hz, 1H), 8.07 (dd, J = 7.7, 1.6 Hz, 1H), 7.50 (dd, J = 7.7, 4.9 Hz, 1H), 7.20-7.11 (m, 2H), 4.72 (d, J = 1.6 Hz, 2H), 3.88 (s, 3H); ES-MS [M + H]⁺ = 353.0/355.0. |
| 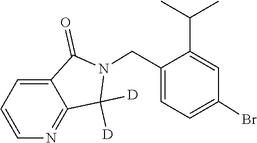 [SMILES: [2H]C1([2H])c2ncccc2C(=O)N1Cc1ccc(Br)cc1C(C)C] | 6-(4-bromo-2- isopropylbenzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 7.48-7.40 (m, 2H), 7.31 (dd, J = 8.2, 2.1 Hz, 1H), 7.10 (d, J = 8.2 Hz, 1H), 4.85 (s, 2H), 3.26 (hept, J = 7.0 Hz, 1H), 1.19 (d, J = 6.8 Hz, 6H); ES-MS [M + H]⁺ = 349.0. |

### Intermediate Example 10. Preparation of 4-(4,4,5,5-tetramethyl-1,3,2-dioxaborolan-2-yl)benzyl)-6,7-dihydro-5H-pyrrolo[3,4-b]pyridin-5-ones

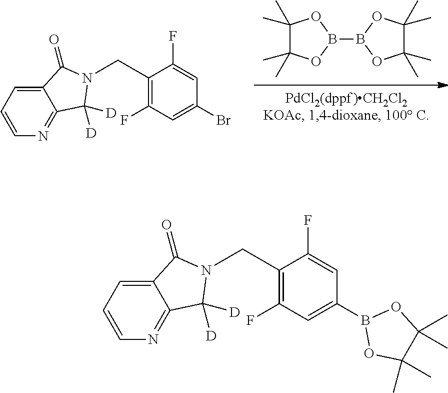

6-(2,6-Difluoro-4-(4,4,5,5-tetramethyl-1,3,2-dioxaborolan-2-yl)benzyl)-6,7-dihydro-5H-pyrrolo[3,4-b]pyridin-5-one-7,7-d₂ (Intermediate 4). 6-(4-Bromo-2,6-difluorobenzyl)-6,7-dihydro-5H-pyrrolo[3,4-b]pyridin-5-one-7,7-d₂ (341 mg, 1.0 mmol, 1.0 eq.), [1,1′-bis(diphenylphosphino)ferrocene]dichloropalladium(II) complex with dichloromethane (81.6 mg, 0.1 mmol, 0.1 eq.), bis(pinocolato)diboron (381 mg, 1.5 mmol, 1.5 eq.) and potassium acetate (294 mg, 3.0 mmol, 3.0 eq.) were combined. Degassed 1,4-dioxane (6.7 mL, 0.15 M) was added. The reaction mixture was evacuated and purged with N2 (3×). After 1 h at 100° C., the reaction mixture was filtered through a pad of Celite®, which was rinsed thoroughly with EtOAc/DCM. The filtrate was concentrated under pressure. Purification using normal phase chromatography on silica gel (0-70% EtOAc/DCM) provided the title compound (295 mg, 76% yield). ¹H NMR (400 MHz, CDCl₃) δ 8.72 (dd, J=5.0, 1.6 Hz, 1H), 8.15 (dd, J=7.7, 1.6 Hz, 1H), 7.45-7.35 (m, 3H), 4.99 (s, 2H), 1.35 (s, 12H); ES-MS [boronic acid]=307.4.

The compounds shown in Table 9 may be prepared similarly to the compound described above, with appropriate starting materials.

TABLE 9

| Structure | Name | ¹H-NMR and/or ES-MS [M + H]⁺ |
|---|---|---|
| 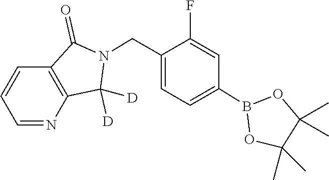 [SMILES: [2H]C1([2H])c2ncccc2C(=O)N1Cc1ccc(B2OC(C)(C)C(C)(C)O2)cc1F] | 6-(2-Fluoro-4-(4,4,5,5- tetramethyl-1,3,2- dioxaborolan-2- yl)benzyl)-6,7-dihydro- 5H-pyrrolo[3,4-b]pyridin- 5-one-7,7-d₂ | ES-MS [M + H]⁺ = 371.4 (boronic ester) and 289.2 (boronic acid) |
| 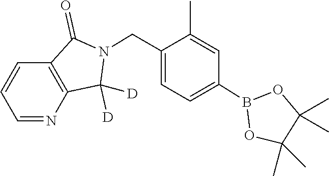 [SMILES: [2H]C1([2H])c2ncccc2C(=O)N1Cc1ccc(B2OC(C)(C)C(C)(C)O2)cc1C] | 6-(2-Methyl-4-(4,4,5,5- tetramethyl-1,3,2- dioxaborolan-2- yl)benzyl)-6,7-dihydro- 5H-pyrrolo[3,4-b]pyridin- 5-one-7,7-d₂ | ES-MS [M + H]⁺ = 367.4 (boronic ester) and 285.4 (boronic acid) |
| 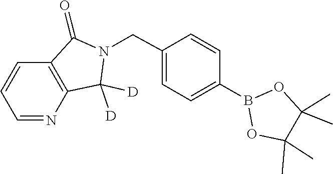 | 6-(4-(4,4,5,5-tetramethyl- 1,3,2-dioxaborolan-2- yl)benzyl)-6,7-dihydro- 5H-pyrrolo[3,4-b]pyridin- 5-one-7,7-d₂ | ES-MS [M + H]⁺ = 270 (boronic acid) |
| 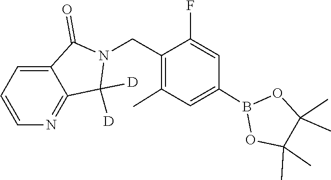 [SMILES: [2H]C1([2H])c2ncccc2C(=O)N1Cc1c(C)cc(B2OC(C)(C)C(C)(C)O2)cc1F] | 6-(2-fluoro-6-methyl-4- (4,4,5,5-tetramethyl- 1,3,2-dioxaborolan-2- yl)benzyl)-6,7-dihydro- 5H-pyrrolo[3,4-b]pyridin- 5-one-7,7-d₂ | ES-MS [M + H]⁺ = 385 (boronic ester) and 303 (boronic acid) |
| 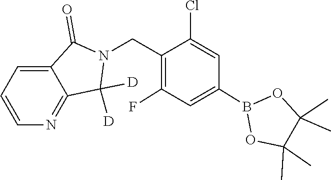 | 6-(2-chloro-6-fluoro-4- (4,4,5,5-tetramethyl- 1,3,2-dioxaborolan-2- yl)benzyl)-6,7-dihydro- 5H-pyrrolo[3,4-b]pyridin- 5-one-7,7-d₂ | ES-MS [M + H]⁺ = 323 (boronic acid) |
| 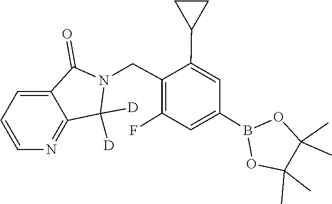 [SMILES: [2H]C1([2H])c2ncccc2C(=O)N1Cc1c(F)cc(B2OC(C)(C)C(C)(C)O2)cc1C1CC1] | 6-(2-cyclopropyl-6- fluoro-4-(4,4,5,5- tetramethyl-1,3,2- dioxaborolan-2- yl)benzyl)-6,7-dihydro- 5H-pyrrolo[3,4-b]pyridin- 5-one-7,7-d₂ | ES-MS [M + H]⁺ = 411.2 (boronic ester) and 329.0 (boronic acid) |
| 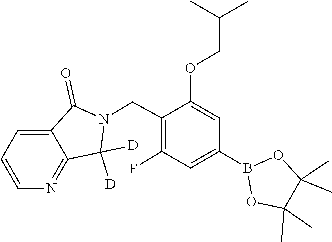 [SMILES: [2H]C1([2H])c2ncccc2C(=O)N1Cc1c(F)cc(B2OC(C)(C)C(C)(C)O2)cc1OCC(C)C] | 6-(2-fluoro-6-isobutoxy- 4-(4,4,5,5-tetramethyl- 1,3,2-dioxaborolan-2- yl)benzyl)-6,7-dihydro- 5H-pyrrolo[3,4-b]pyridin- 5-one-7,7-d₂ | ES-MS [M + H]⁺ = 361.2 (bornic ester) and 343.3 (bornic acid) |
| 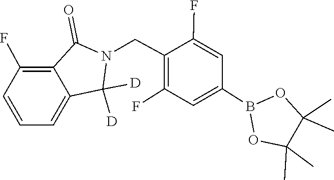 | 2-(2,6-difluoro-4- (4,4,5,5-tetramethyl- 1,3,2-dioxaborolan-2- yl)benzyl)-7- fluoroisoindolin-1-one- 3,3-d₂ | ES-MS [M + H]⁺ = 324.0 (bornic acid) |
| 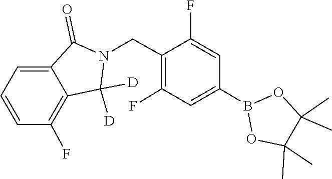 | 2-(2,6-difluoro-4- (4,4,5,5-tetramethyl- 1,3,2-dioxaborolan-2- yl)benzyl)-4- fluoroisoindolin-1-one- 3,3-d₂ | ES-MS [M + H]⁺ = 324.0 (bornic acid) |
| 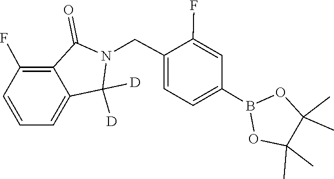 | 7-fluoro-2-(2-fluoro-4- (4,4,5,5-tetramethyl- 1,3,2-dioxaborolan-2- yl)benzyl)isoindolin-1- one-3,3-d₂ | ES-MS [M + H]⁺ = 306.0 (bornic acid) |
| 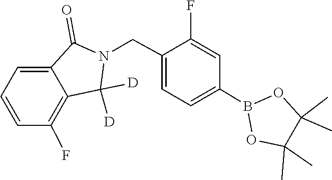 | 4-fluoro-2-(2-fluoro-4- (4,4,5,5-tetramethyl- 1,3,2-dioxaborolan-2- yl)benzyl)isoindolin-1- one-3,3-d | ES-MS [M + H]⁺ = 306.0 (bornic acid) |

### Intermediate Example 11: Preparation of 4-bromo-7-(difluoromethoxy)-1H-indazole

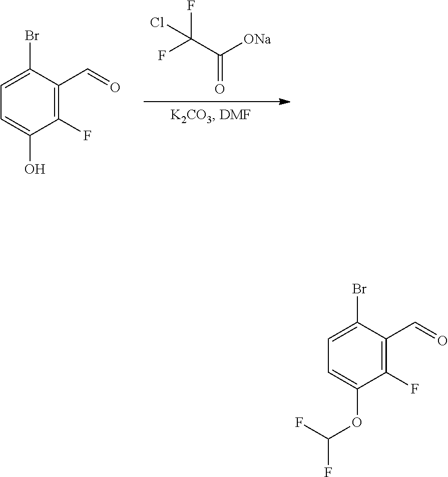

Step 1: 6-Bromo-3-(difluoromethoxy)-2-fluorobenzaldehyde. A mixture of 6-bromo-2-fluoro-3-hydroxybenzaldehyde (219.0 mg, 1.0 mmol), sodium chlorodifluoroacetate (305 mg, 2.0 mmol) and potassium carbonate (140.2 mg, 1.0 mml) in DMF (3.2 mL) and water (0.4 mL) was stirred at 100° C. for 2 h. After cooling to rt, the reaction mixture was diluted with EtOAc and water. The layers were separated and the aqueous layer was extracted with EtOAc (3×). The combined extracts were washed with brine, dried (Na₂SO₄), filtered, and concentrated. Purification using normal phase chromatography on silica gel (0-50% EtOAc) provided the title compound (142 mg, 53% yield). ¹H NMR (400 MHz, CDCl₃) δ 10.32 (d, J=1.1 Hz, 1H), 7.48 (dd, J=8.8, 1.8 Hz, 1H), 7.36 (ddt, J=8.7, 7.7, 0.9 Hz, 1H), 6.58 (t, J=72.3 Hz, 1H); ES-MS [M+H]⁺=not ionizable.

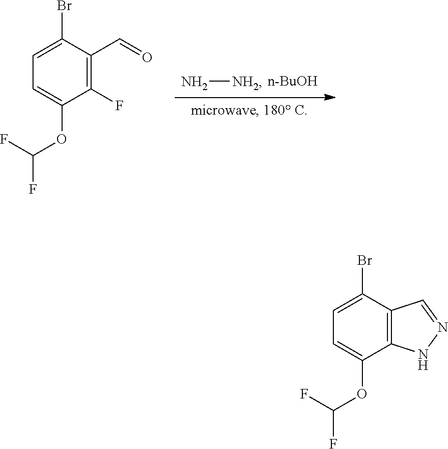

Step 2: 4-Bromo-7-(difluoromethoxy)-1H-indazole. A mixture of 6-bromo-3-(difluoromethoxy)-2-fluorobenzaldehyde (36 mg, 0.13 mmol) and hydrazine (21 μL, 0.66 mmol) in n-butanol (1.0 mL) was subjected to a microwave reactor at 180° C. for 30 min. The resulting mixture was concentrated under reduced pressure. Purification using normal phase chromatography on silica gel (0-100% EtOAc) provided the title compound (23 mg, 65% yield). ¹H NMR (400 MHz, DMSO) δ 8.12 (s, 1H), 7.41 (t, J=72.3 Hz, 1H), 7.33 (d, J=8.0 Hz, 1H), 7.10 (d, J=8.0 Hz, 1H); ES-MS [M+H]⁺=262.9/264.9.

The compounds shown in Table 10 may be prepared similarly to the compound described above, with appropriate starting materials.

TABLE 10

| Structure | Name | ¹H-NMR and/or ES-MS [M + H]⁺ |
|---|---|---|
| 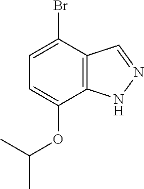 | 4-bromo-7-isopropoxy- 1H-indazole | ES-MS [M + H]⁺ = 255.0/257.0. |
| 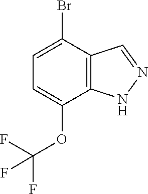 [SMILES: FC(F)(F)Oc1ccc(Br)c2cn[nH]c12] | 4-bromo-7- (trifluoromethoxy)-1H- indazole | ¹H NMR (400 MHz, DMSO) δ 8.21 (s, 1H), 7.41 (d, J = 8.0 Hz, 1H), 7.35 (dq, J = 8.0, 1.4 Hz, 1H), NH proton is exchangable; ES-MS [M + H]⁺ = 281.0/283.0. |
| 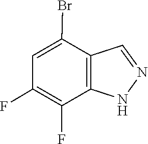 [SMILES: Fc1cc(Br)c2cn[nH]c2c1F] | 4-bromo-6,7-difluoro-1H- indazole | ¹H NMR (400 MHz, DMSO) δ 8.18 (d, J = 3.1 Hz, 1H), 7.57 (dd, J = 10.3, 5.9 Hz, 1H), NH proton is exchangable; ES-MS [M + H]⁺ = 233.0/235.0. |

### Intermediate Example 12. Preparation of 2-methyl-2H-indazoles

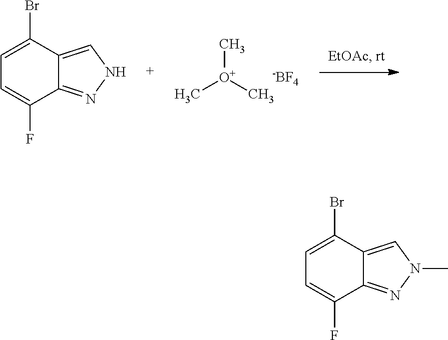

4-Bromo-7-fluoro-2-methyl-2H-indazole. To a solution of 4-bromo-7-fluoro-2H-indazole (215.02 mg, 1.0 mmol, 1.0 eq.) in EtOAc (5.0 mL, 0.2 M) was added trimethyloxonium tetrafluoroborate (295.8 mg, 2.0 mmol, 2.0 eq.). After 16 h at rt, the mixture was adjusted to neutral pH with a sat. solution of NaHCO₃ and extracted with EtOAc (3×). The combined extracts were washed with brine, dried over Na₂SO₄, filtered, and concentrated under reduced pressure. Purification using normal phase chromatography on silica gel (0-50% EtOAc/hexanes) provided the title compound (150 mg, 65% yield). ¹H NMR (400 MHz, CDCl₃) δ 7.98 (d, J=2.5 Hz, 1H), 7.14 (dd, J=7.9, 3.6 Hz, 1H), 6.85 (dd, J=10.8, 7.9 Hz, 1H), 4.28 (s, 3H); 2D NOESY (400 MHz, CDCl₃): a cross-peak occurred between N-methyl (CH₃, s at 4.28 ppm) and indazole CH (d at 7.98 ppm); ES-MS [M+H]⁺=229.2/231.2.

The compounds shown in Table 11 may be prepared similarly to the compound described above, with appropriate starting materials.

TABLE 11

| Structure | Name | ¹H-NMR and/or ES-MS [M + H]⁺ |
|---|---|---|
| 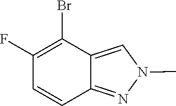 | 4-bromo-5-fluoro-2-methyl- 2H-indazole | ¹H NMR (400 MHz, CDCl₃) δ 7.95 (s, 1H), 7.63 (ddd, J = 9.2, 4.1, 0.9 Hz, 1H), 7.15 (t, J = 9.1 Hz, 1H), 4.25 (s, 3H); 2D NOESY (400 MHz, CDCl₃): a cross-peak occurred between N-methyl (CH₃, s at 4.25 ppm) and indazole CH (s at 7.95 ppm); ES-MS [M + H]⁺ = 229.2.23.1.2 |
| 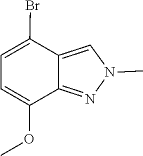 | 4-bromo-7-methoxy-2-methyl- 2H-indazole | ¹H NMR (400 MHz, MHz, CDCl₃) δ 7.86 (s, 1H), 7.11 (d, J = 7.9 Hz, 1H), 6.43 (d, J = 7.9 Hz, 1H), 4.22 (s, 3H), 3.99 (s, 3H); 2D NOESY (400 MHz, CDCl₃): a cross- peak occurred between N-methyl (CH₃, s at 4.22 ppm) and indazole CH (s at 7.86 ppm); ES-MS [M + H]⁺ = 241.2/243.2. |
| 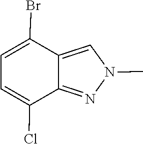 | 4-bromo-7-chloro-2-methyl- 2H-indazole | ¹H NMR (400 MHz, CDCl₃) δ 8.00 (s, 1H), 7.24-7.15 (m, 2H), 4.30 (s, 3H); 2D NOESY (400 MHz, CDCl₃): a cross-peak occurred between N-methyl (s at 4.30 ppm) and indazole CH (s at 8.00 ppm); ES-MS [M + H]⁺ = 245.2/24.2. |
| 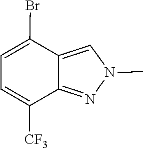 | 4-bromo-2-methyl-7- (trifluoromethyl)-2H-indazole | ¹H NMR (400 MHz, CDCl₃) δ 8.07 (s, 1H), 7.49 (dd, J = 7.5, 1.1 Hz, 1H), 7.32 (dd, J = 7.5, 0.8 Hz, 1H), 4.32 (s, 3H); 2D NOESY (400 MHz, CDCl₃): a cross-peak occurred between N-methyl (CH₃, s at 4.32 ppm) and indazole CH (s at 8.07 ppm); ES-MS [M + H]⁺ = 279.2/281.2. |
| 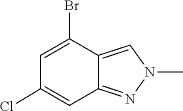 | 4-bromo-6-chloro-2-methyl- 2H-indazole | ¹H NMR (400 MHz, CDCl₃) δ 7.90 (s, 1H), 7.62 (dd, J = 1.4, 0.9 Hz, 1H), 7.23 (d, J = 1.5 Hz, 1H), 4.21 (s, 3H); 2D NOESY (400 MHz, CDCl₃): a cross-peak occurred between N-methyl (CH₃, s, at 4.21 ppm) and indazole CH (s at 7.90 ppm); ES-MS [M + H]⁺ = 245.2/247.2. |
| 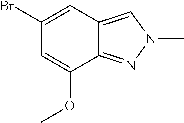 | 5-bromo-7-methoxy-2-methyl- 2H-indazole | ¹H NMR (400 MHz, CDCl₃) δ 7.79 (s, 1H), 7.37 (d, J = 1.4 Hz, 1H), 6.63 (d, J = 1.4 Hz, 1H), 4.19 (s, 3H), 4.00 (s, 3H); 2D NOESY (400 MHz, CDCl₃): a cross-peak occurred between N-methyl (CH₃, s, at 4.19 ppm) and indazole CH (s at 7.79 ppm); ES-MS [M + H]⁺ = 241.0/243.0. |
| 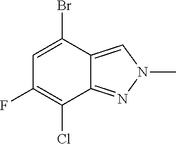 | 4-bromo-7-chloro-6-fluoro-2- methyl-2H-indazole | ¹H NMR (400 MHz, CDCl₃) δ 7.96 (s, 1H), 7.16 (d, J = 8.9 Hz, 1H), 4.25 (s, 3H); 2D NOESY (400 MHz, CDCl₃): a cross-peak occurred between N-methyl (CH₃, s, at 4.25 ppm) and indazole CH (s at 7.96 ppm); ES-MS [M + H]⁺ = 262.8/264.8. |
| 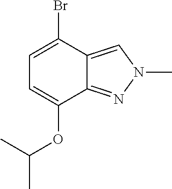 | 4-bromo-7-isopropoxy-2- methyl-2H-indazole | ¹H NMR (400 MHz, CDCl₃) δ 7.85 (s, 1H), 7.09 (d, J = 7.9 Hz, 1H), 6.45 (dd, J = 7.9, 0.6 Hz, 1H), 4.84-4.74 (m, 1H), 4.23 (s, 3H), 1.47 (d, J = 6.0 Hz, 6H); ES-MS [M + H]⁺ = 269.0/271.0 |
| 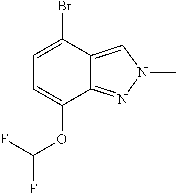 | 4-bromo-7-(difluoromethoxy)- 2-methyl-2H-indazole | ¹H NMR (400 MHz, DMSO) δ ¹H NMR (400 MHz, DMSO) δ 8.53 (s, 1H), 7.47 (t, J = 74.2 Hz, 1H), 7.25 (d, J = 7.9 Hz, 1H), 6.97-6.91 (m, 1H), 4.21 (s, 3H); ES- MS [M + H]⁺ = 277.0/279.0. |
| 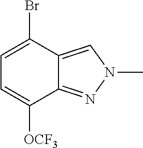 [SMILES: Cn1cc2c(Br)ccc(OC(F)(F)F)c2n1] | 4-bromo-2-methyl-7- (trifluoromethoxy)-2H- indazole | ¹H NMR (400 MHz, DMSO) δ 8.60 (d, J = 0.6 Hz, 1H), 7.32 (d, J = 7.9 Hz, 1H), 7.24-7.17 (m, 1H), 4.23 (s, 3H); ES-MS [M + H]⁺ = 294.8/296.9 |
| 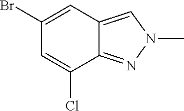 | 5-bromo-7-chloro-2-methyl- 2H-indazole | ¹H NMR (400 MHz, CDCl₃) δ 7.91 (s, 1H), 7.72 (d, J = 1.6 Hz, 1H), 7.42 (d, J = 1.6 Hz, 1H), 4.25 (s, 3H); ES- MS [M + H]⁺ = 245.2/247.2. |
| 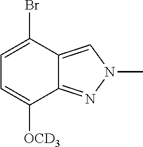 | 4-bromo-7-(methoxy-d₃)-2- methyl-2H-indazole | ¹H NMR (400 MHz, CDCl₃) δ 7.86 (s, 1H), 7.10 (d, J = 7.8 Hz, 1H), 6.43 (d, J = 7.9 Hz, 1H), 4.22 (s, 3H); ES- MS [M + H]⁺ = 244.0/246.0. |
| 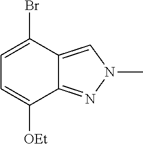 | 4-bromo-7-ethoxy-2-methyl- 2H-indazole | ¹H NMR (400 MHz, CDCl₃) δ 7.86 (s, 1H), 7.09 (d, J = 7.9 Hz, 1H), 6.43 (d, J = 7.9 Hz, 1H), 4.22 (d, J = 6.1 Hz, 5H), 1.56 (d, J = 7.2 Hz, 3H); ES-MS [M + H]⁺ = 255/257 |
| 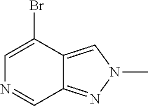 | 4-bromo-2-methyl-2H- pyrazolo[3,4-c]pyridine | ¹H NMR (400 MHz, DMSO) δ 9.13 (t, J = 0.7 Hz, 1H), 8.61 (s, 1H), 8.22 (d, J = 0.6 Hz, 1H), 4.28 (s, 3H); ES- MS [M + H]⁺ = 212/214. |
| 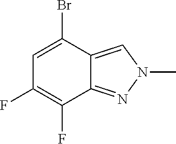 | 4-bromo-6,7-difluoro-2- methyl-2H-indazole | ¹H NMR (400 MHz, DMSO) δ 8.60 (d, J = 2.5 Hz, 1H), 7.51 (dd, J = 10.5, 5.9 Hz, 1H), 4.20 (s, 3H); ES-MS [M + H]⁺ = 247/249. |
| 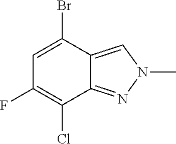 | 4-bromo-7-chloro-6-fluoro-2- methyl-2H-indazole | ¹H NMR (400 MHz, CDCl₃) δ 7.96 (s, 1H), 7.16 (d, J = 8.9 Hz, 1H), 4.25 (s, 3H); ES-MS [M + H]⁺ = 263.0/265.0 |
| 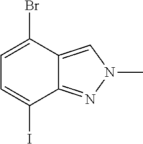 | 4-bromo-7-iodo-2-methyl-2H- indazole | ¹H NMR (400 MHz, DMSO) δ 8.62 (d, J = 0.5 Hz, 1H), 7.63 (d, J = 7.5 Hz, 1H), 7.07 (d, J = 7.5 Hz, 1H); ES-MS [M + H]⁺ = 336.8/338.8 |

### Intermediate Example 13. Preparation of 2-(methyl-d₃)-2H-indazoles

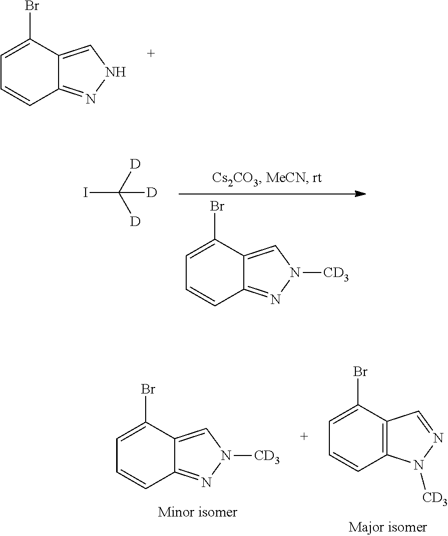 [SMILES: Brc1cccc2n[nH]cc12 ; [2H]C([2H])([2H])I ; [2H]C([2H])([2H])n1cc2c(Br)cccc2n1 ; "Cs2CO3, MeCN, rt" ; [2H]C([2H])([2H])n1cc2c(Br)cccc2n1 ; [2H]C([2H])([2H])n1ncc2c(Br)cccc21]

4-Bromo-2-(methyl-d₃)-2H-indazole (Minor Isomer) To a suspension of 4-bromo-2H-indazole (3.94 g, 20.0 mmol, 1.0 eq.) and cesium carbonate (13.1 g, 40 mmol, 2.0 eq.) in MeCN (100 mL, 0.2 M) was added iodomethane-d₃ (2.49 mL, 40 mmol, 2.0 eq.). After 16 h at rt, the reaction mixture was diluted with deionized H₂O and extracted with EtOAc (3×). The combined organic extracts were washed with H₂O, brine, dried over Na₂SO₄, filtered, and concentrated. The crude material was purified by normal phase chromatography on silica gel (0-30% EtOAc/hexanes) to provide the title isomer (1.50 g, 35% yield). ¹H NMR (400 MHz, CDCl₃) δ 7.92 (s, 1H), 7.63 (d, J=8.6 Hz, 1H), 7.21 (d, J=7.2 Hz, 1H), 7.13 (dd, J=8.5, 7.3 Hz, 1H); ¹H NMR was also confirmed with the proteo compound, CAS #590417-93-9; ES-MS [M+H]⁺=214.2/216.3.

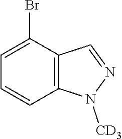 [SMILES: [2H]C([2H])([2H])n1ncc2c(Br)cccc21]

4-Bromo-1-(methyl-d₃)-1H-indazole (Major isomer): ¹H NMR (400 MHz, CDCl₃) δ 7.99 (d, J=0.6 Hz, 1H), 7.34 (d, J=8.2 Hz, 1H), 7.30 (dd, J=7.3, 0.6 Hz, 1H), 7.23 (dd, J=8.1, 7.4 Hz, 1H); ES-MS [M+H]⁺=214.2/216.3.

The compounds shown in Table 12 may be prepared similarly to the compound described above, with appropriate starting materials.

TABLE 12

| Structure | Name | ¹H-NMR and/or ES-MS [M + H]⁺ |
|---|---|---|
| 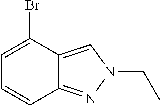 | 4-bromo-2-ethyl-2H- indazole | ¹H NMR (400 MHz, CDCl₃) δ 7.88 (d, J = 0.9 Hz, 1H), 7.58 (dt, J = 8.6, 0.8 Hz, 1H), 7.16 (dd, J = 7.2, 0.7 Hz, 1H), 7.07 (dd, J = 8.6, 7.2 Hz, 1H), 4.42 (q, J = 7.3 Hz, 2H), 1.58 (t, J = 7.4 Hz, 3H); 2D NOESY (400 MHz, CDCl₃): a cross- peak occurred between N-ethyl (CH₂, q at 4.42 ppm) and indazole CH (d at 7.88 ppm); ES-MS |
| | | [M + H]⁺ = 225.4/227.3. |
| 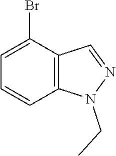 | 4-bromo-1-ethyl-1H- indazole | ¹H NMR (400 MHz, CDCl₃) δ 7.93 (d, J = 0.9 Hz, 1H), 7.29 (dt, J = 8.3 0.9 Hz, 1H), 7.22 (dd, J = 7.4, 0.8 Hz, 1H), 7.15 (dd, J = 8.3, 7.3 Hz, 1H), 4.35 (q, J = 7.3 Hz, 2H), 1.44 (t, J = 7.3 Hz, 3H); ES-MS [M + H]⁺ = 225.4/227.3. |
| 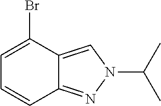 | 4-bromo-2-isopropyl-2H- indazole | ¹H NMR (400 MHz, CDCl₃) δ 7.90 (d, J = 0.9 Hz, 1H), 7.59 (dt, J = 8.6, 0.8 Hz, 1H), 7.16 (dd, J = 7.2, 0.7 Hz, 1H), 7.06 (dd, J = 8.6, 7.2 Hz, 1H), 4.73 (h, J = 6.7 Hz, 1H), 1.60 (d, J = 6.7 Hz, 6H); 2D NOESY (400 MHz, CDCl₃): a cross- peak occurred between isopropyl CH (h at 4.73 ppm) and indazole CH (d at 7.90 ppm); ES-MS |
| | | [M + H]⁺ = 239.3/241.2. |
| 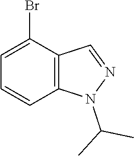 | 4-bromo-1-isopropyl-1H- indazole | ¹H NMR (400 MHz, CDCl₃) δ 7.95 (d, J = 0.9 Hz, 1H), 7.32 (dd, J = 8.3, 0.9 Hz, 1H), 7.22 (dd, J = 7.4, 0.7 Hz, 1H), 7.14 (dd, J = 8.3, 7.4 Hz, 1H), 4.75 (h, J = 6.7 Hz, 1H), 1.53 (d, J = 6.7 Hz, 6H); ES-MS [M + H]⁺ = 239.3/241.2. |
| 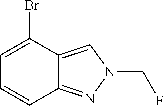 [SMILES: FCn1cc2c(Br)cccc2n1] | 4-bromo-2-(fluoromethyl)- 2H-indazole | ¹H NMR (400 MHz, CDCl₃) δ 8.22 (s, 1H), 7.68 (d, J = 8.7 Hz, 1H), 7.30 (d, J = 7.0 Hz, 1H), 7.20 (dd, J = 8.7, 7.2 Hz, 1H), 6.31 (d, J = 51.2, 2H); 2D NOESY (400 MHz, CDCl₃): a cross-peak occurred between CH₂ (d at 6.31 ppm) and indazole CH (s at 8.22 ppm); ES-MS [M + H]⁺ = 229.2/231.2. |
| 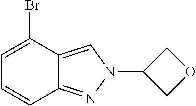 [SMILES: Brc1cccc2nn(C3COC3)cc12] | 4-bromo-2-(oxetan-3-yl)- 2H-indazole | ¹H NMR (400 MHz, CDCl₃) δ 8.14 (d, J = 1.0 Hz, 1H), 7.69 (d, J = 8.6 Hz, 1H), 7.27 (d, J = 7.9 Hz, 1H), 7.18 (dd, J = 8.6, 7.2 Hz, 1H), 5.73 (tt, J = 7.5, .0 Hz, 1H), 5.25-5.13 (m, 4H); 2D NOESY (400 MHz, CDCl₃): a cross-peak occurred between oxetane CH (m at 5.73 ppm) and indazole CH (s at 8.14 ppm); ES-MS |
| | | [M + H]⁺ = 253.2/255.2. |
| 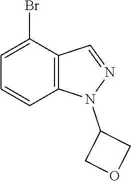 [SMILES: Brc1cccc2c1cnn2C1COC1] | 4-bromo-1-(oxetan-3-yl)- 1H-indazole | ¹H NMR (400 MHz, CDCl₃) δ 8.11 (s, 1H), 7.48 (d, J = 8.4 Hz, 1H), 7.34 (d, J = 7.4 Hz, 1H), 7.26 (dd, J = 8.3, 7.5 Hz, 1H), 5.77 (tt, J = 7.8, 6.2 Hz, 1H), 5.29 (dd, J = 6.5, 6.5 Hz, 2H), 5.14 (dd, J = 6.5, 6.5 Hz, 2H); ES-MS [M + H]⁺ = 253.2/255.2. |
| 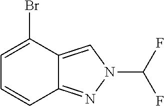 [SMILES: FC(F)n1cc2c(Br)cccc2n1] | 4-bromo-2-(difluoromethyl)- 2H-indazole | ES-MS [M + H]⁺ = 247.2/249.2. |
| 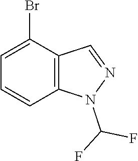 [SMILES: FC(F)n1ncc2c(Br)cccc21] | 4-bromo-1-(difluoromethyl)- 1H-indazole | ES-MS [M + H]⁺ = 247.2/249.2. |
| 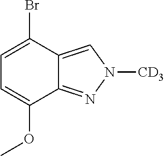 [SMILES: [2H]C([2H])([2H])n1cc2c(Br)ccc(OC)c2n1] | 4-bromo-7-methoxy-2- (methyl-d₃)-2H-indazole | ¹H NMR (400 MHz, CDCl₃) δ 7.86 (s, 1H), 7.11 (d, J = 7.9 Hz, 1H), 6.43 (d, J = 7.9 Hz, 1H), 3.99 (s, 3H); N-methyl (s, 4.22 ppm) not observed when comparing with ¹H NMR of 4-bromo-7- methoxy-2-methyl-2H-indazole; ES-MS [M + H]⁺ = 244.3/246.3. |
| 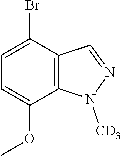 [SMILES: [2H]C([2H])([2H])n1ncc2c(Br)ccc(OC)c21] | 4-bromo-7-methoxy-1- (methyl-d₃)-1H-indazole | ¹H NMR (400 MHz, CDCl₃) δ 7.89 (s, 1H), 7.12 (d, J = 8.0 Hz, 1H), 6.54 (d, J = 8.0 Hz, 1H), 3.94 (s, 3H); ES-MS [M + H]⁺ = 244.3/246.3. |
| 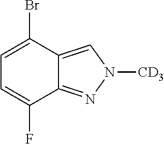 [SMILES: [2H]C([2H])([2H])n1cc2c(Br)ccc(F)c2n1] | 4-bromo-7-fluoro-2-(methyl- d₃)-2H-indazole | ¹H NMR (400 MHz, CDCl₃) δ 7.96 (d, J = 2.6 Hz, 1H), 7.12 (dd, J = 7.9, 3.6 Hz, 1H), 6.83 (dd, J = 10.8, 7.9 Hz, 1H); N-methyl (s, 4.28 ppm) not observed when comparing with ¹H NMR of 4-bromo-7-fluoro-2-methyl-2H- indazole; ES-MS [M + H]⁺ = 232.2/234.2. |
| 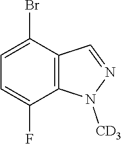 [SMILES: [2H]C([2H])([2H])n1ncc2c(Br)ccc(F)c21] | 4-bromo-7-fluoro-1-(methyl- d₃)-1H-indazole | ¹H NMR (400 MHz, CDCl₃) δ 8.01 (d, J = 2.2 Hz, 1H), 7.20 (dd, J = 8.1, 3.5 Hz, 1H), 6.96 (dd, J = 11.4, 8.1 Hz, 1H); ES-MS [M + H]⁺ = 232.2/234.2. |
| 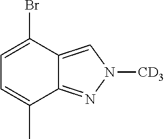 | 4-bromo-7-methyl-2- (methyl-d₃)-2H-indazole | ¹H NMR (400 MHz, CDCl₃) δ 7.90 (s, 1H), 7.13 (d, J = 7.2 Hz, 1H), 6.94-6.87 (m, 1H), 2.57 (d, J = 1.1 Hz, 3H); N-methyl (s, 4.24 ppm) not observed when comparing with ¹H NMR of 4- bromo-2,7-dimethyl-2H-indazole, CAS# 1159511-88-2; ES-MS [M + H]⁺ = 228.3/230.3. |
| 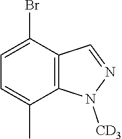 | 4-bromo-7-methyl-1- (methyl-d₃)-1H-indazole | ¹H NMR (400 MHz, CDCl₃) δ 7.93 (s, 1H), 7.14 (d, J = 7.4 Hz, 1H), 6.93 (dd, J = 7.5, 1.0 Hz, 1H), 2.71 (d, J = 1.0 Hz, 3H); ES-MS [M + H]⁺ = 228.3/230.3. |
| 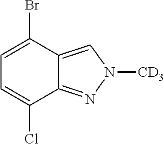 [SMILES: [2H]C([2H])([2H])n1cc2c(Br)ccc(Cl)c2n1] | 4-bromo-7-chloro-2- (methyl-d₃)-2H-indazole | ¹H NMR (400 MHz, CDCl₃) δ 7.98 (s, 1H), 7.17 (dd, J = 10.8, 7.7 Hz, 2H); ES-MS [M + H]⁺ = 248.0/250.0. |
| 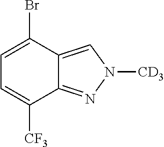 [SMILES: [2H]C([2H])([2H])n1cc2c(Br)ccc(C(F)(F)F)c2n1] | 4-bromo-2-(methyl-d₃)-7- (trifluoromethyl)-2H- indazole | ¹H NMR (400 MHz, CDCl₃) δ 8.05 (s, 1H), 7.46 (dd, J = 7.6, 1.1 Hz, 1H), 7.29 (d, J = 7.5, 1H); ES-MS [M + H]⁺ = 282.0/284.0. |
| 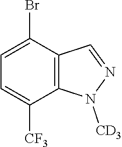 [SMILES: [2H]C([2H])([2H])n1ncc2c(Br)ccc(C(F)(F)F)c21] | 4-bromo-1-(methyl-d₃)-7- (trifluoromethyl)-1H- indazole | ¹H NMR (400 MHz, CDCl₃) δ 8.11 (s, 1H), 7.58 (dd, J = 7.8, 0.9 Hz, 1H), 7.36 (dd, J = 7.9, 0.8 Hz, 1H); ES-MS [M + H]⁺ = 282.0/284.0. |
| 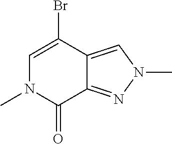 | 4-bromo-2,6-dimethyl-2,6- dihydro-7H-pyrazolo[3,4- c]pyridin-7-one | ¹H NMR (400 MHz, CDCl₃) δ 7.64 (s, 1H), 6.97 (s, 1H), 4.15 (s, 3H), 3.57 (s, 3H); ES-MS [M + H]⁺ = 243.9. |
| 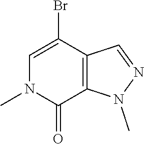 | 4-bromo-1,6-dimethyl-1,6- dihydro-7H-pyrazolo[3,4- c]pyridin-7-one | ¹H NMR (400 MHz, CDCl₃ ) δ 7.71 (s, 1H), 7.01 (s, 1H), 4.38 (s, 3H), 3.57 (s, 3H); ES-MS [M + H]⁺ = 241.9/243.9. |
| 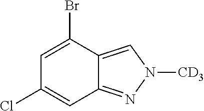 [SMILES: [2H]C([2H])([2H])n1cc2c(Br)cc(Cl)cc2n1] | 4-bromo-6-chloro-2- (methyl-d₃)-2H-indazole | ¹H NMR (400 MHz, CDCl₃) δ 7.90 (d, J = 0.9 Hz, 1H), 7.63 (dd, J = 1.5, 0.9 Hz, 1H), 7.23 (d, J = 1.5 Hz, 1H); ES-MS [M + H]⁺ = 247.9/249.9. |
| 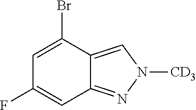 [SMILES: [2H]C([2H])([2H])n1cc2c(Br)cc(F)cc2n1] | 4-bromo-6-fluoro-2-(methyl- d₃)-2H-indazole | ¹H NMR (400 MHz, CDCl₃) δ 7.91 (d, J = 0.9 Hz, 1H), 7.26-7.24 (m, 1H), 7.10 (dd, J = 8.8, 1.9 Hz, 1H); ES-MS [M + H]⁺ = 231.9/233.9. |
| 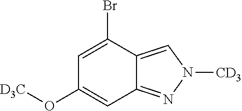 [SMILES: [2H]C([2H])([2H])Oc1cc(Br)c2cn(C([2H])([2H])[2H])nc2c1] | 4-bromo-6-(methoxy-d₃)-2- (methyl-d₃)-2H-indazole | ¹H NMR (400 MHz, CDCl₃) δ 7.81 (d, J = 0.9 Hz, 1H), 6.99 (d, J = 1.9 Hz, 1H), 6.89 (dd, J = 1.9, 0.9 Hz, 1H); ES-MS [M + H]⁺ = 249.0/249.9. |
| 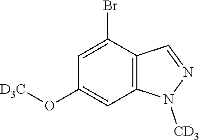 [SMILES: [2H]C([2H])([2H])Oc1cc(Br)c2cnn(C([2H])([2H])[2H])c2c1] | 4-bromo-6-(methoxy-d₃)-1- (methyl-d₃)-1H-indazole | ¹H NMR (400 MHz, CDCl₃) δ 7.86 (d, J = 1.0 Hz, 1H), 6.99 (d, J = 1.8 Hz, 1H), 6.64 (dd, J = 1.9, 1.0 Hz, 1H); ES-MS [M + H]⁺ = 249.0/249.9. |
| 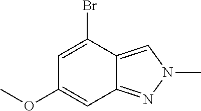 | 4-bromo-6-methoxy-2- methyl-2H-indazole | ¹H NMR (400 MHz, CDCl₃) δ 7.80 (s, 1H), 6.98 (d, J = 1.9 Hz, 1H), 6.89 (dd, J = 1.9, 0.9 Hz, 1H), 4.16 (s, 3H), 3.84 (s, 3H); ES-MS [M + H]⁺ = 242.9. |
| 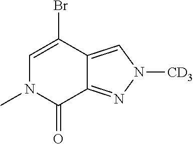 [SMILES: [2H]C([2H])([2H])n1cc2c(Br)cn(C)c(=O)c2n1] | 4-bromo-6-methyl-2- (methyl-d₃)-2,6-dihydro-7H- pyrazolo[3,4-c]pyridin-7-one | ¹H NMR (400 MHz, CDCl₃) δ 7.64 (s, 1H), 6.96 (s, 1H), 3.57 (s, 3H); ES-MS [M + H]⁺ = 246.9. |
| 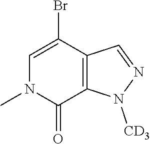 [SMILES: [2H]C([2H])([2H])n1ncc2c(Br)cn(C)c(=O)c21] | 4-bromo-6-methyl-1- (methyl-d₃)-1,6-dihydro-7H- pyrazolo[3,4-c]pyridin-7-one | ¹H NMR (400 MHz, CDCl₃) δ 7.71 (s, 1H), 7.01 (s, 1H), 3.57 (s, 3H); ES-MS [M + H]⁺ = 246.9. |
| 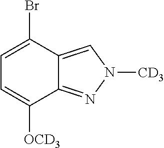 | 4-bromo-7-(methoxy-d₃)-2- (methyl-d₃)-2H-indaozle | ¹H NMR (400 MHz, CDCl₃) δ 7.86 (s, 1H), 7.10 (d, J = 7.9 Hz, 1H), 6.42 (d, J = 7.9 Hz, 1H); ES-MS [M + H]⁺ = 248.9. |
| 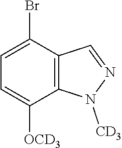 | 4-bromo-7-(methoxy-d₃)-1- (methyl-d₃)-1H-indazole | ¹H NMR (400 MHz, CDCl₃) δ 7.89 (s, 1H), 7.11 (d, J = 8.0 Hz, 1H), 6.53 (d, J = 8.0 Hz, 1H); ES-MS [M + H]⁺ = 248.9. |
| 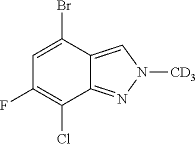 [SMILES: [2H]C([2H])([2H])n1cc2c(Br)cc(F)c(Cl)c2n1] | 4-bromo-7-chloro-6-fluoro- 2-(methyl-d₃)-2H-indazole | ¹H NMR (400 MHz, CDCl₃) δ 7.96 (s, 1H), 7.16 (d, J = 8.9 Hz, 1H); ES-MS [M + H]⁺ = 266.0/268.0 |
| 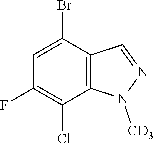 [SMILES: [2H]C([2H])([2H])n1ncc2c(Br)cc(F)c(Cl)c21] | 4-bromo-7-chloro-6-fluoro- 1-(methyl-d₃)-1H-indazole | ¹H NMR (400 MHz, CDCl₃) δ 7.94 (s, 1H), 7.18 (d, J = 8.7 Hz, 1H); ES-MS [M + H]⁺ = 266.0/268.0 |

### Intermediate Example 14. Preparation of 4-bromo-7-alkoxy-2-methyl-2H-indazoles via Alkylation Method

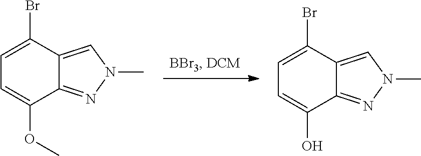

4-Bromo-2-methyl-2H-indazol-7-ol. To a solution of 4-bromo-7-methoxy-2-methyl-2H-indazole (482 mg, 2.0 mmol) in DCM (10 mL) at 0° C. was added a solution of boron tribromide (1M in DCM, 5.0 mL, 5.0 mmol). The reaction mixture was stirred at rt. After 2 h, the reaction mixture was cooled back down to 0° C. and ice water was slowly added and stirred for 30 min. The layers were separated. The aqueous layer was extracted with DCM (3×). The combined organic layers were dried over Na₂SO₄, filtered, and concentrated. The crude material was carried forward without further purification. ¹H NMR (400 MHz, DMSO) δ 10.04 (s, 1H), 8.26 (s, 1H), 7.00 (d, J=7.8 Hz, 1H), 6.42 (d, J=7.8 Hz, 1H), 4.16 (s, 3H); ES-MS [M+H]⁺=227.0/229.0.

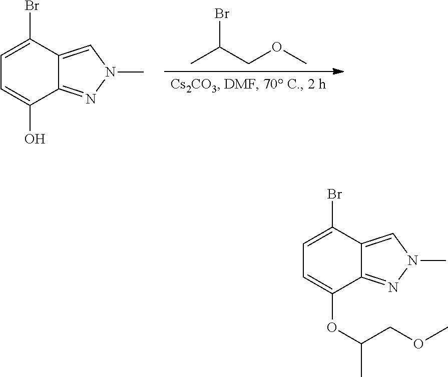

4-Bromo-7-((1-methoxypropan-2-yl)oxy)-2-methyl-2H-indazole. To a solution of 4-bromo-2-methyl-2H-indazol-7-ol (150 mg, 0.66 mmol) and cesium carbonate (694 mg, 2.12 mmol) in DMF (3.5 mL) was added 2-bromo-1-methoxypropane (150 uL, 1.36 mmol). Resulting mixture was stirred at 70° C. After 2 h, mixture was cooled to room temperature, diluted with water and extracted with EtOAc (3×). The combined organics were washed with brine, dried over Na₂SO₄, filtered, and concentrated under reduced pressure. The crude residue was purified via silica gel chromatography to provide the title compound (89 mg, 45% yield). ¹H NMR (400 MHz, CDCl₃) δ 7.82 (s, 1H), 7.05 (d, J=7.9 Hz, 1H), 6.50 (d, J=7.9 Hz, 1H), 4.80 (sextet, J=6.0 Hz, 1H), 4.18 (s, 3H), 3.73 (dd, J=10.1, 5.8 Hz, 1H), 3.53 (dd, J=10.1, 5.1 Hz, 1H), 3.39 (s, 3H), 1.40 (d, J=6.3 Hz, 3H); ES-MS [M+H]⁺=299.0/301.0.

The compounds shown in Table 13 may be prepared similarly to the compound described above, with appropriate starting materials.

TABLE 13

| Structure | Name | ¹H-NMR and/or ES-MS [M + H]⁺ |
|---|---|---|
| 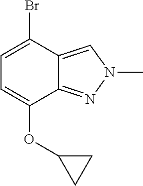 | 4-bromo-7-cyclopropoxy-2- methyl-2H-indazole | ¹H NMR (400 MHz, DMSO) δ 8.32 (s, 1H), 7.16 (d, J = 7.9 Hz, 1H), 6.83 (d, J = 7.9 Hz, 1H), 3.98 (dq, J = 6.1, 3.0 Hz, 1H), 0.89- 0.68 (m, 4H); ES-MS [M + H]⁺ = 267.0/269.0. |
| 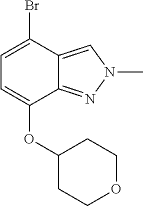 [SMILES: Cn1cc2c(Br)ccc(OC3CCOCC3)c2n1] | 4-bromo-2-methyl-7- ((tetrahydro-2H-pyran-4- yl)oxy)-2H-indazole | ¹H NMR (400 MHz, CDCl₃) δ 7.88 (s, 1H), 7.08 (d, J = 7.9 Hz, 1H), 6.50 (d, J = 7.9 Hz, 1H), 4.75 (tt, J = 8.5, 4.1 Hz, 1H), 4.24 (s, 3H), 4.07 (dt, J = 11.9, 4.4 Hz, 2H), 3.58 (ddd, J = 12.0, 9.5, 2.9 Hz, 2H), 2.17-2.10 (m, 2H), 2.01-1.92 (m, 2H); ES-MS [M + H]⁺ = 311.0/313.0 |
| 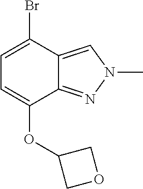 [SMILES: Cn1cc2c(Br)ccc(OC3COC3)c2n1] | 4-bromo-2-methyl-7-(oxetan- 3-yloxy)-2H-indazole | ¹H NMR (400 MHz, CDCl₃) δ 7.90 (s, 1H), 7.05 (d, J = 7.8 Hz, 1H), 6.05 (d, J = 7.8 Hz, 1H), 5.46-5.36 (m, 1H), 5.06-4.91 (m, 4H), 4.25 (s, 3H); ES-MS [M + H]⁺ = 283.0/285.0 |
| 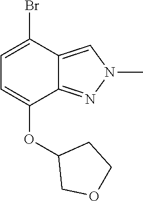 [SMILES: Cn1cc2c(Br)ccc(OC3CCOC3)c2n1] | 4-bromo-2-methyl-7- ((tetrahydrofuran-3-yl)oxy)- 2H-indazole | ¹H NMR (400 MHz, DMSO) δ 8.33 (s, 1H), 7.11 (d, J = 7.8 Hz, 1H), 6.54 (d, J = 7.9 Hz, 1H), 5.23 (ddt, J = 6.3, 4.0, 1.8 Hz, 1H), 4.16 (s, 3H), 3.93 (dd, J = 10.2, 4.5 Hz, 1H), 3.91- 3.82 (m, 2H), 3.78 (td, J = 8.3, 4.6 Hz, 1H), 2.31-2.19 (m, 1H), 2.10-1.99 (m, 1H); ES- MS [M + H]⁺ = 297.0/299.0. |
| 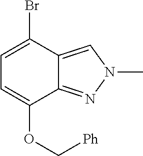 [SMILES: Cn1cc2c(Br)ccc(OCc3ccccc3)c2n1] | 7-(benzyloxy)-4-bromo-2- methyl-2H-indazole | ¹H NMR (400 MHz, DMSO) δ 8.33 (s, 1H), 7.52-7.46 (m, 2H), 7.41 (ddd, J = 8.0, 7.0, 1.0 Hz, 2H), 7.36 (d, J = 7.1 Hz, 1H), 7.12 (d, J = 7.9 Hz, 1H), 6.66 (d, J = 7.9 Hz, 1H), 5.24 (s, 2H), 4.16 (s, 3H); ES-MS [M + H]⁺ = 317.0/319.0. |
| 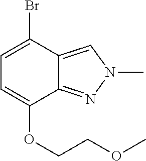 [SMILES: COCCOc1ccc(Br)c2cn(C)nc12] | 4-bromo-7-(2- methoxyethoxy)-2-methyl-2H- indazole | ¹H NMR (400 MHz, DMSO) δ 8.32 (s, 1H), 7.11 (d, J = 7.9 Hz, 1H), 6.56 (d, J = 7.9 Hz, 1H), 4.27-4.20 (m, 2H), 4.16 (s, 3H), 3.76- 3.69 (m, 2H), 3.33 (s, 3H); ES-MS [M + H]⁺ = 285.0/287.0 |
| 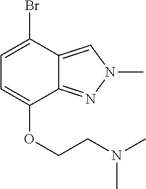 [SMILES: CN(C)CCOc1ccc(Br)c2cn(C)nc12] | 2-((4-bromo-2-methyl-2H- indazol-7-yl)oxy)-N,N- dimethylethan-1-amine | ¹H NMR (400 MHz, DMSO) δ 8.31 (s, 1H), 7.10 (d, J = 7.9 Hz, 1H), 6.57 (d, J = 7.9 Hz, 1H), 4.18 (t, J = 5.8 Hz, 3H), 4.16 (s, 2H), 2.23 (s, 6H); ES-MS [M + H]⁺ = 298.0/300.0 |
| 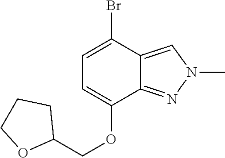 [SMILES: Cn1cc2c(Br)ccc(OCC3CCCO3)c2n1] | 4-bromo-2-methyl-7- ((tetrahydrofuran-2- yl)methoxy)-2H-indazole | ¹H NMR (400 MHz, CDCl₃) δ 7.81 (s, 1H), 7.04 (d, J = 7.9 Hz, 1H), 6.45 (d, J = 7.9 Hz, 1H), 4.42-4.35 (m, 1H), 4.20-4.14 (m, 4H), 4.07 (dd, J = 9.9, 5.5 Hz, 1H), 3.93-3.87 (m, 1H), 3.81-3.74 (m, 1H), 2.17-2.06 (m, 1H), 2.01-1.87 (m, 2H), 1.87-1.76 (m, 1H); ES- MS [M + H]⁺ = 311.0/313.0 |
| 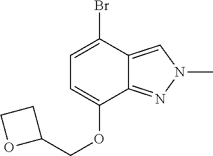 [SMILES: Cn1cc2c(Br)ccc(OCC3CCO3)c2n1] | 4-bromo-2-methyl-7-(oxetan- 2-ylmethoxy)-2H-indazole | ¹H NMR (400 MHz, CDCl₃) δ 7.82 (s, 1H), 7.05 (d, J = 7.9 Hz, 1H), 6.51 (d, J = 7.9 Hz, 1H), 5.25-5.18 (m, 1H), 4.71-4.59 (m, 2H), 4.37-4.29 (m, 2H), 4.16 (s, 3H), 2.85-2.75 (m, 1H), 2.72-2.62 (m, 1H); ES-MS [M + H]⁺ = 297.0/299.0 |
| 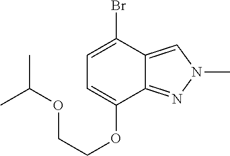 [SMILES: CC(C)OCCOc1ccc(Br)c2cn(C)nc12] | 4-bromo-7-(2- isopropoxyethoxy)-2-methyl- 2H-indazole | ¹H NMR (400 MHz, CDCl₃) δ 7.78 (s, 1H), 7.02 (d, J = 7.9 Hz, 1H), 6.43 (d, J = 7.9 Hz, 1H), 4.25 (t, J = 5.8 Hz, 2H), 4.14 (s, 3H), 3.85 (t, J = 5.8 Hz, 2H), 3.64 (septet, J = 6.1 Hz, 1H), 1.14 (d, J = 6.1 Hz, 6H); ES-MS [M + H]⁺ = 313.0/315.0 |
| 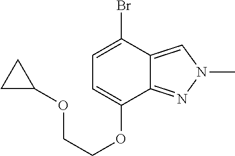 [SMILES: Cn1cc2c(Br)ccc(OCCOC3CC3)c2n1] | 4-bromo-7-(2- cyclopropoxyethoxy)-2- methyl-2H-indazole | ¹H NMR (400 MHz, CDCl₃) δ 7.79 (s, 1H), 7.03 (d, J = 7.9 Hz, 1H), 6.41 (d, J = 7.9 Hz, 1H), 4.26 (t, J = 5.3 Hz, 2H), 4.16 (s, 3H), 3.94 (t, J = 5.3 Hz, 2H), 3.37 (tt, J = 6.0, 3.0 Hz, 1H), 0.62-0.52 (m, 2H), 0.51-0.40 (m, 2H); ES-MS [M + H]⁺ = 311.0/313.0 |
| 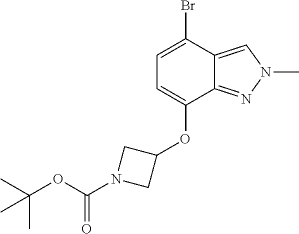 [SMILES: Cn1cc2c(Br)ccc(OC3CN(C(=O)OC(C)(C)C)C3)c2n1] | tert-butyl 3-((4-bromo-2- methyl-2H-indazol-7- yl)oxy)azetidine-1-carboxylate | ¹H NMR (400 MHz, CDCl₃) δ 7.85 (s, 1H), 7.01 (d, J = 7.8 Hz, 1H), 6.09 (d, J = 7.9 Hz, 1H), 5.03 (tt, J = 6.4, 4.2 Hz, 1H), 4.30 (ddd, J = 9.8, 6.4, 1.1 Hz, 2H), 4.18 (s, 3H), 4.14 (dd, J = 10.0, 4.2 Hz, 2H), 1.40 (s, 9H); ES-MS [M + H]⁺ = 382.0/384.0 |
| 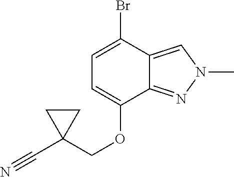 [SMILES: Cn1cc2c(Br)ccc(OCC3(C#N)CC3)c2n1] | 1-(((4-bromo-2-methyl-2H- indazol-7- yl)oxy)methyl)cyclopropane-1- carbonitrile | ¹H NMR (400 MHz, DMSO-d₆) δ 8.36 (s, 1H), 7.12 (d, J = 7.9 Hz, 1H), 6.54 (d, J = 7.9 Hz, 1H), 4.19 (s, 3H), 4.18 (s, 2H), 1.44-1.38 (m, 2H), 1.23-1.16 (m, 2H); ES-MS [M + H]⁺ = 306.0/308.0 |
| 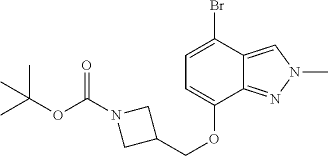 [SMILES: Cn1cc2c(Br)ccc(OCC3CN(C(=O)OC(C)(C)C)C3)c2n1] | tert-butyl 3-(((4-bromo-2- methyl-2H-indazol-7- yl)oxy)methyl)azetidine-1- carboxylate | ¹H NMR (400 MHz, CDCl₃) δ 7.83 (s, 1H), 7.05 (d, J = 7.9 Hz, 1H), 6.42 (d, J = 7.9 Hz, 1H), 4.27 (d, J = 7.4 Hz, 2H), 4.18 (s, 3H), 4.11 (t, J = 8.5 Hz, 2H), 3.76 (dd, J = 8.8, 5.0 Hz, 2H), 3.18-3.03 (m, 1H), 1.41 (s, 9H); ES-MS [M]⁺ = 396.0/398.0 |
| 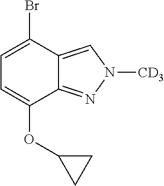 [SMILES: [2H]C([2H])([2H])n1cc2c(Br)ccc(OC3CC3)c2n1] | 4-bromo-7-cyclopropoxy-2- (methyl-d₃)-2H-indazole | ¹H NMR (400 MHz, CDCl₃) δ 7.86 (s, 1H), 7.13 (d, J = 7.9 Hz, 1H), 6.82 (d, J = 7.9 Hz, 1H), 3.89 (tt, J = 6.1, 3.0 Hz, 1H), 0.98-0.93 (m, 2H), 0.86-0.80 (m, 2H); ES-MS [M + H]⁺ = 270.0/272.0 |
| 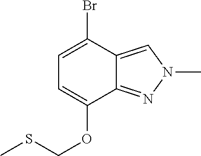 | 4-bromo-2-methyl-7- ((methylthio)methoxy)-2H- indazole | ¹H NMR (400 MHz, CDCl₃) δ 7.88 (s, 1H), 7.11 (d, J = 7.9 Hz, 1H), 6.57 (d, J = 7.9 Hz, 1H), 5.43 (s, 2H), 4.22 (s, 3H), 2.30 (s, 3H); ES-MS [M + H]⁺ = 287.0/289.0 |

### Intermediate Example 15. Preparation of 4-bromo-7-alkoxy-2-methyl-2H-indazoles via Mitsunobu Method

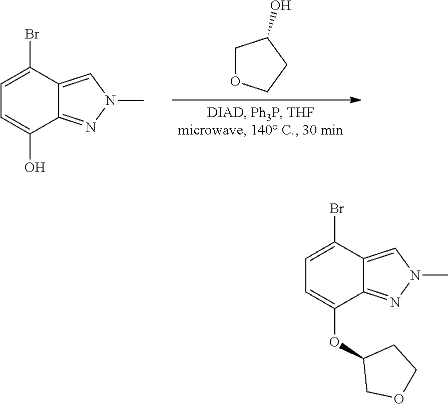

(S)-4-Bromo-2-methyl-7-((tetrahydrofuran-3-yl)oxy)-2H-indazole. A microwave vial was charged with 4-bromo-2-methyl-2H-indazol-7-ol (212 mg, 0.93 mmol) and THF (2.5 mL). To this solution was added (R)-tetrahydrofuran-3-ol (115 μL, 1.43 mmol), triphenylphosphine (540 mg, 2.06 mmol) and diisopropyl azodicarboxylate (410 mg, 2.03 mmol). The resulting mixture was subjected to microwave irradiation at 140° C. After 30 min, the solvent was removed and the crude residue was purified via silica gel chromatography on silica gel (0-100% EtOAc/hexanes) to provide the title compound (278 mg, 99% yield). ¹H NMR (400 MHz, CDCl₃) δ 7.88 (s, 1H), 7.09 (d, J=7.9 Hz, 1H), 6.38 (d, J=7.9 Hz, 1H), 5.20-5.14 (m, 1H), 4.24 (s, 3H), 4.17-4.01 (m, 3H), 3.94 (td, J=8.2, 4.3 Hz, 1H), 2.37-2.21 (m, 2H); ES-MS [M+H]⁺=297/299.

The compounds shown in Table 14 may be prepared similarly to the compound described above, with appropriate starting materials.

TABLE 14

| Structure | Name | ¹H-NMR and/or ES-MS [M + H]⁺ |
|---|---|---|
| 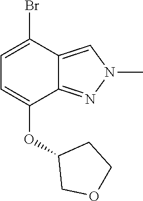 [SMILES: Cn1cc2c(Br)ccc(O[C@@H]3CCOC3)c2n1] | (R)-4-bromo-2-methyl-7- ((tetrahydrofuran-3-yl)oxy)- 2H-indazole | ¹H NMR (400 MHz, CDCl₃) δ 7.88 (s, 1H), 7.09 (d, J = 7.9 Hz, 1H), 6.38 (d, J = 7.9 Hz, 1H), 5.20-5.14 (m, 1H), 4.24 (s, 3H), 4.17-4.01 (m, 3H), 3.94 (td, J = 8.2, 4.3 Hz, 1H), 2.37- 2.21 (m, 2H); ES-MS [M + H]⁺ = 297/299 |
| 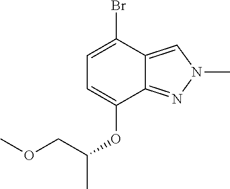 | (R)-4-bromo-7-((1- methoxypropan-2-yl)oxy)-2- methyl-2H-indazole | ¹H NMR (400 MHz, CDCl₃) δ 7.80 (s, 1H), 7.04 (d, J = 7.9 Hz, 1H), 6.48 (d, J = 8.0 Hz, 1H), 4.79 (sextet, J = 6.1 Hz, 1H), 4.16 (s, 3H), 3.72 (dd, J = 10.1, 5.8 Hz, 1H), 3.52 (dd, J = 10.1, 5.0 Hz, 1H), 3.37 (s, 3H), 1.39 (d, J = 6.3 Hz, 3H); ES-MS [M + H]⁺ = 299/301; |
| 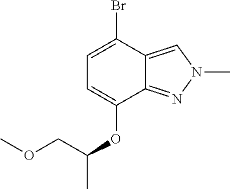 | (S)-4-bromo-7-((1- methoxypropan-2-yl)oxy)-2- methyl-2H-indazole | ¹H NMR (400 MHz, CDCl₃) δ 7.80 (s, 1H), 7.04 (d, J = 7.9 Hz, 1H), 6.48 (d, J = 7.9 Hz, 1H), 4.79 (sextet, J = 6.1 Hz, 1H), 4.16 (s, 3H), 3.71 (dd, J = 10.1, 5.8 Hz, 1H), 3.52 (dd, J = 10.1, 5.0 Hz, 1H), 3.37 (s, 3H), 1.39 (d, J = 6.3 Hz, 3H); ES-MS [M + H]⁺ = 399/301 |
| 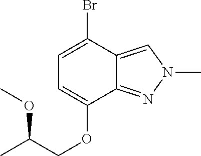 [SMILES: CO[C@H](C)COc1ccc(Br)c2cn(C)nc12] | (R)-4-bromo-7-(2- methoxypropoxy)-2-methyl- 2H-indazole | ¹H NMR (400 MHz, CDCl₃) δ 7.83 (s, 1H), 7.06 (d, J = 7.9 Hz, 1H), 6.44 (d, J = 7.9 Hz, 1H), 4.24-4.15 (m, 4H), 4.01 (dd, J = 9.9, 5.0 Hz, 1H), 3.88 (sextet, J = 6.2 Hz, 1H), 3.45 (s, 3H), 1.30 (d, J = 6.3 Hz, 3H); ES-MS [M]⁺ = 299/301; ES-MS [M + H]⁺ = 302 |

### Intermediate Example 16. Preparation of 4-bromo-2-methyl-7-((methylsulfonyl)methoxy)-2H-indazole

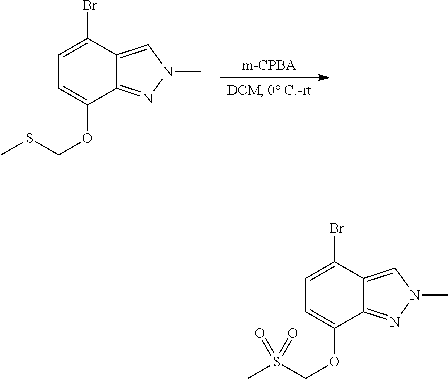

4-Bromo-2-methyl-7-((methylsulfonyl)methoxy)-2H-indazole: To a solution of 4-bromo-2-methyl-7-((methylthio)methoxy)-2H-indazole (104 mg, 0.362 mmol) in DCM (2 mL) at 0° C. was added 3-chloroperoxybenzoic acid (137.49 mg, 0.797 mmol). After 20 min, the mixture was warmed to rt. After 3 h, the reaction was quenched with sat. NaHCO₃ and extracted with DCM. The organic layers were combined, washed with brine, passed through a phase separator, and concentrated under reduced pressure to provide the title compound (105.4 mg, 91% yield), which was used without further purification. ES-MS [M]=319/321.

### Intermediate Example 17. Preparation of 4-Bromo-7-((1-butylazetidin-3-yl)oxy)-2-methyl-2H-indazole

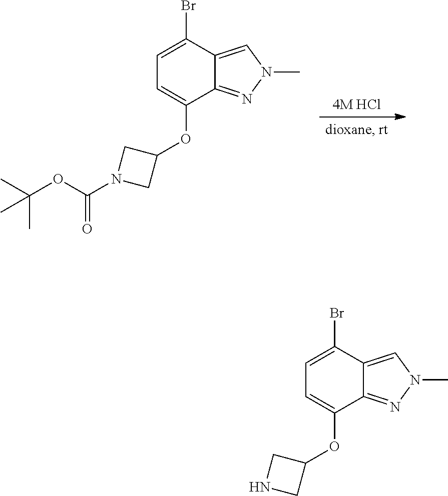 [SMILES: Cn1cc2c(Br)ccc(OC3CN(C(=O)OC(C)(C)C)C3)c2n1 ; "4M HCl" ; "dioxane, rt" ; Cn1cc2c(Br)ccc(OC3CNC3)c2n1]

7-(Azetidin-3-yloxy)-4-bromo-2-methyl-2H-indazole hydrochloride: To a solution of tert-butyl 3-((4-bromo-2-methyl-2H-indazol-7-yl)oxy)azetidine-1-carboxylate (352 mg, 0.921 mmol) in 1,4-dioxane (4 mL) at 0° C. was added hydrochloric acid (4M solution in 1,4-dioxane, 1 mL). After stirring at 0° C. for 10 min, the solution was warmed to rt. The solvent was removed under reduced pressure to afford the title compound which was used without further purification. ES-MS [M+H]⁺=282/284.

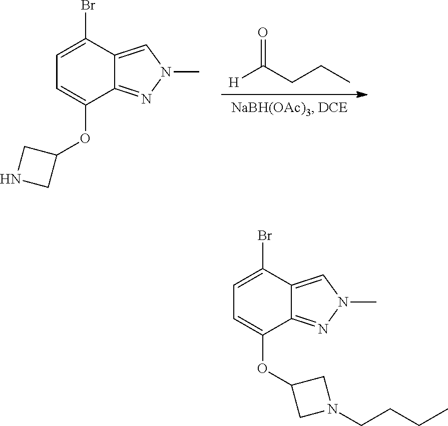

4-Bromo-7-((1-butylazetidin-3-yl)oxy)-2-methyl-2H-indazole: To a solution of 7-(azetidin-3-yloxy)-4-bromo-2-methyl-2H-indazole hydrochloride (53.4 mg, 0.168 mmol) in 1,2-dichloroethane (1 mL) was added butyraldehyde (30 μL, 0.332 mmol) and sodium triacetoxyborohydride (191.6 mg, 0.904 mmol). The resulting solution was stirred overnight. The reaction was quenched with sat. NaHCO₃ and extracted with CHCl₃:IPA (3:1). The organic layers were combined, washed with brine, passed through a phase separator, and concentrated under reduced pressure to provide the title compound which was used without further purification. ES-MS [M+H]⁺=338/340.

The compounds shown in Table 15 may be prepared similarly to the compound described above, with appropriate starting materials.

TABLE 15

| Structure | Name | ¹H-NMR and/or ES-MS [M + H]⁺ |
|---|---|---|
| 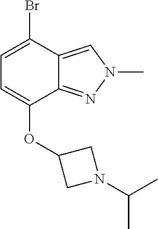 [SMILES: CC(C)N1CC(Oc2ccc(Br)c3cn(C)nc23)C1] | 4-bromo-7-((1- isopropylazetidin-3-yl)oxy)-2- methyl-2H-indazole | ES-MS [M + H]⁺ = 324/326 |
| 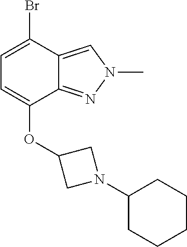 [SMILES: Cn1cc2c(Br)ccc(OC3CN(C4CCCCC4)C3)c2n1] | 4-bromo-7-((1- cyclohexylazetidin-3-yl)oxy)- 2-methyl-2H-indazole | ES-MS [M + H]⁺ = 364/366; |

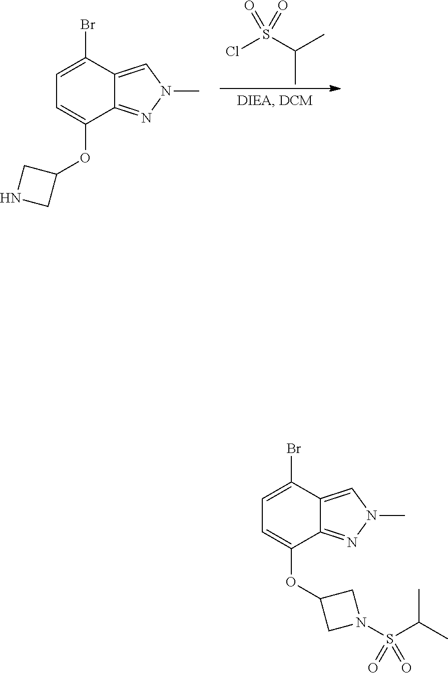

4-Bromo-7-((1-(isopropylsulfonyl)azetidin-3-yl)oxy)-2-methyl-2H-indazole: To a solution of 7-(azetidin-3-yloxy)-4-bromo-2-methyl-2H-indazole hydrochloride (50 mg, 0.157 mmol) in DCM (1 mL) was added N,N-diisopropylethylamine (275 μL, 1.569 mmol) and isopropylsulfonyl chloride (22 μL, 0.196 mmol). The resulting solution was stirred overnight. The reaction mixture was quenched with sat. NaHCO₃ and extracted with DCM (3×). The organic layers were combined, washed with brine, passed through a phase separator, and concentrated under reduced pressure to provide the title compound which was used without further purification. ES-MS [M+H]⁺=388/390.

### Intermediate Example 18. Preparation of 4-bromo-2-methyl-7-(methylthio)-2H-indazole and (4-bromo-2-methyl-2H-indazol-7-yl)(imino)(methyl)-λ⁶-sulfanone

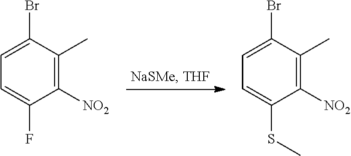

Step 1: (4-Bromo-3-methyl-2-nitrophenyl)(methyl)sulfane. To a solution of 1-bromo-4-fluoro-2-methyl-3-nitrobenzene (540 mg, 2.31 mmol) in THE (11.5 mL) was added a solution of sodium methanthiolate (21% in H₂O, 3.01 mL, 9.23 mmol). The resulting mixture was stirred at 50° C. After 16 h, the mixture was diluted with water and extracted with DCM (3×). The combined extracts were passed through a phase separator and concentrated. Purification using normal phase chromatography on silica gel (0-30% EtOAc/hexanes) provided the title compound (496 mg, 82% yield). ¹H NMR (400 MHz, CDCl₃) δ 7.62 (d, J=8.5 Hz, 1H), 7.16 (d, J=8.7 Hz, 1H), 2.47 (s, 3H), 2.36 (s, 3H); ES-MS [M+H]⁺=261.9/263.9.

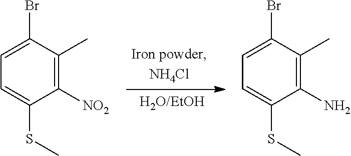

Step 2: 3-Bromo-2-methyl-6-(methylthio)aniline. Iron powder (1.03 g, 18.3 mmol) and ammonium chloride (1.47 g, 27.5 mmol) were combined in water (8.08 mL) and ethanol (22.4 mL). The resulting mixture was stirred at 70° C. and (4-bromo-3-methyl-2-nitrophenyl)(methyl)sulfane (1.2 g, 4.58 mmol) was added portionwise. The reaction mixture was stirred at 100° C. for 2 h. After cooling to below refluxing temperature, the mixture was filtered through a pad of Celite and washed with warm MeOH. The filtrate was concentrated under reduced pressure. The resulting residue was re-dissolved in water and extracted with DCM (3×). The combined extracts were dried (Na₂SO₄), filtered, and concentrated to provide the title compound (1.03 g, 96% yield), which was used without further purification. ES-MS [M+H]⁺=231.9/233.9.

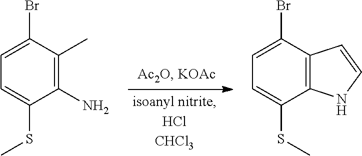

Step 3: 4-Bromo-7-(methylthio)-1H-indazole. To a solution of 3-bromo-2-methyl-6-(methylthio)aniline (1.02 g, 4.39 mmol) in chloroform (22 mL) at 0° C. was added acetic anhydride (0.95 mL, 10.1 mmol). After 5 min, the reaction mixture was warmed to rt. After 1 h, potassium acetate (521 mg, 5.23 mmol) was added followed by isoamyl nitrite (1.48 mL, 11.0 mmol). The resulting mixture was heated at reflux for 20 h. After cooling to rt, the solvent was evaporated to yield a brown solid. The solid was re-dissolved in water which was removed by evaporation and concentrated HCl (12 M aq. solution, 1.8 mL, 22 mmol) was added. The reaction mixture was stirred at 50° C. for 90 min, cooled to 0° C. and adjusted to pH˜14 using 50% KOH aq. solution. The solution mixture was extracted with iPA/CHCl₃ (1:3 v/v, 3×). The combined extracts were passed through a phase separator and concentrated. Purification using normal phase chromatography on silica gel (0-50% EtOAc/hexanes) provided the title compound (636 mg, 60% yield). ¹H NMR (400 MHz, DMSO) δ 8.07 (s, 1H), 7.32 (d, J=7.7 Hz, 1H), 7.17 (d, J=7.8 Hz, 1H), 2.58 (s, 3H), NH proton not observable; ES-MS [M+H]⁺=242.9/244.9.

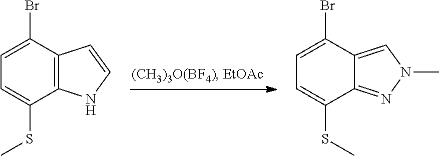

Step 4: 4-Bromo-2-methyl-7-(methylthio)-2H-indazole was prepared similarly to 4-bromo-7-fluoro-2-methyl-2H-indazole. ¹H NMR (400 MHz, DMSO) δ 8.39 (s, 1H), 7.22 (d, J=7.6 Hz, 1H), 6.90 (d, J=7.6 Hz, 1H), 4.18 (s, 3H), 2.53 (s, 3H); ES-MS [M+H]*=256.9/258.9.

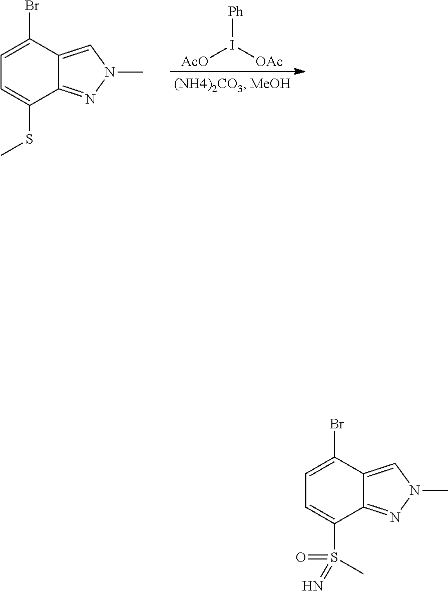

Step 5: (4-Bromo-2-methyl-2H-indazol-7-yl)(imino)(methyl)-X⁶-sulfanone. To a solution of 4-bromo-2-methyl-7-(methylthio)-2H-indazole (25.7 mg, 0.10 mmol) in MeOH (1 mL), was added ammonium carbonate (44 mg, 0.46 mmol) and iodobenzene diacetate (96.6 mg, 0.30 mmol). The resulting solution was stirred rt. After 45 min, the reaction mixture was quenched with sat. NaHCO₃ and extracted with DCM (3×). The organic layers were combined, washed with brine, passed through a phase separator, and concentrated. Purification using normal phase chromatography on silica gel (0-10% MeOH/DCM) provided the title compound. ¹H NMR (400 MHz, DMSO) δ 8.68 (s, 1H), 7.72 (d, J=7.6 Hz, 1H), 7.47 (d, J=7.6 Hz, 1H), 4.43-4.38 (m, 1H), 4.26 (s, 3H); 2.50 (s, 3H); ES-MS [M+H]⁺=288.0/290.0.

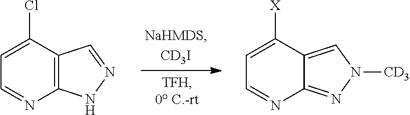

4-Chloro-2-(methyl-d₃)-2H-pyrazolo[3,4-b]pyridine. To a solution of 4-chloro-1H-pyrazolo[3,4-b]pyridine (117 mg, 0.76 mmol) in THE (3 mL) at 0° C., was added sodium bis(trimethylsilyl)amide (1M solution in THF, 900 μL, 0.90 mmol). After 15 min, iodomethane-d₃ (95 μL, 1.5 mmol) was added dropwise to the solution and continued to stir at 0° C. for additional 15 min before warming to room temperature. After 2.5 h, the reaction was quenched with sat. NH₄Cl and extracted with EtOAc (3×). The organic layers were combined, washed with brine, dried over Na₂SO₄, filtered, and concentrated under reduced pressure. The crude residue was purified via silica gel chromatography to provide the title compound (93.5 mg, 72%). ¹H NMR (400 MHz, CDCl₃) δ 8.58 (d, J=4.7 Hz, 1H), 8.01 (s, 1H), 7.08 (d, J=4.7 Hz, 1H); ES-MS [M+H]⁺=171

The compounds shown in Table 16 may be prepared similarly to the compound described above, with appropriate starting materials.

TABLE 16

| Structure | Name | ¹H-NMR and/or ES-MS [M + H]⁺ |
|---|---|---|
| 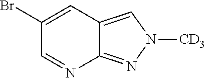 [SMILES: [2H]C([2H])([2H])n1cc2cc(Br)cnc2n1] | 5-bromo-2-(methyl-d₃)-2H- pyrazolo[3,4-b]pyridine | ¹H NMR (400 MHz, DMSO) δ 8.59 (d, J = 2.4 Hz, 1H), 8.51 (d, J = 2.4 Hz, 1H), 8.42 (s, 1H); ES-MS [M + H]⁺ = 215/217. |
| 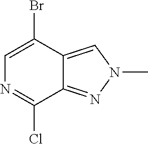 | 4-bromo-7-chloro-2-methyl- 2H-pyrazolo[3,4-c]pyridine | Using Iodomethane as alkylation reagent ¹H NMR (400 MHz, DMSO) δ 8.76 (s, 1H), 8.06 (s, 1H), 4.30 (s, 3H); ES-MS [M + H]⁺ = 247.9. |

### Intermediate Example 19. Preparation of 4-bromo-6-(difluoromethyl)-2-methyl-indazole

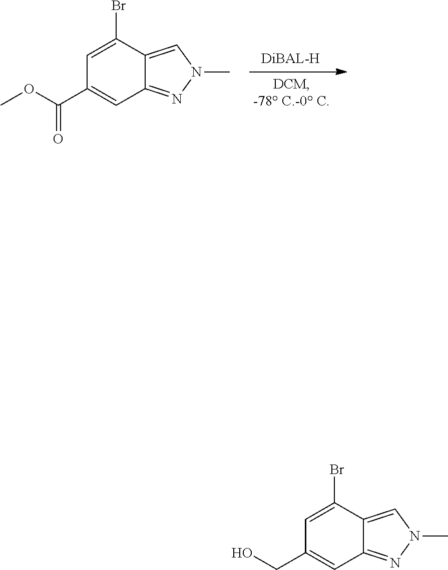

(4-Bromo-2-methyl-indazol-6-yl)methanol: To a solution of methyl 4-bromo-2-methylindazole-6-carboxylate (500 mg, 1.86 mmol) in DCM (8 mL) was slowly added diisobutylaluminum hydride solution (4.65 mL, 4.65 mmol, 1.0 M in THF, 2.5 eq) at −78° C. After 30 min, the reaction was slowly warmed to rt. After 16 h, the reaction was cooled to 0° C. and quenched by water dropwise. When gas evolution had ceased, the ice bath was removed. The residue was extracted with DCM (3×), washed with brine. The combined organic phase was dried (Na₂SO₄) and filtered through phase separator. The filtrate was concentrated to give title compound (422.0 mg, 94% yield) which was used in the next step without further purification. ¹H NMR (400 MHz, CDCl₃) δ 7.91 (d, J=2.2 Hz, 1H), 7.61-7.56 (m, 1H), 7.30 (d, J=2.5 Hz, 1H), 4.75 (d, J=1.2 Hz, 2H), 4.23 (d, J=2.7 Hz, 3H); ES-MS [M+H]⁺=243.0.

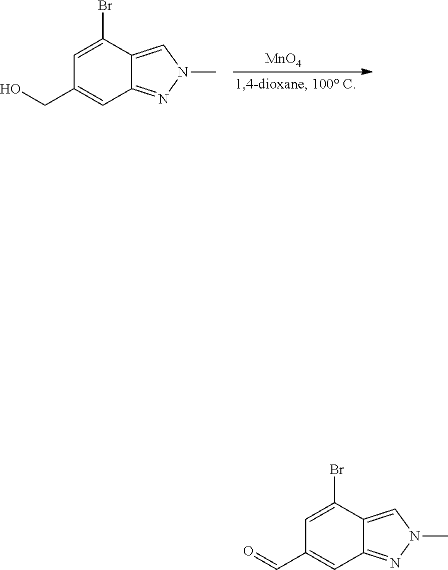

4-Bromo-2-methyl-indazole-6-carbaldehyde. To a solution of (4-bromo-2-methyl-indazol-6-yl)methanol (422.0 mg, 1.75 mmol) in 1,4-dioxane (15 mL) was added activated manganese (IV) oxide (4.56 g, 52.5 mmol) at rt. The mixture was heated to 100° C. After 16 h, the reaction was cooled to rt, filtered through Celite®, and washed with EtOAc. The filtrate was concentrated to give the title compound (199.3 mg, 48% yield), which was used in the next step without further purification; ES-MS [M+H]⁺=241.0.

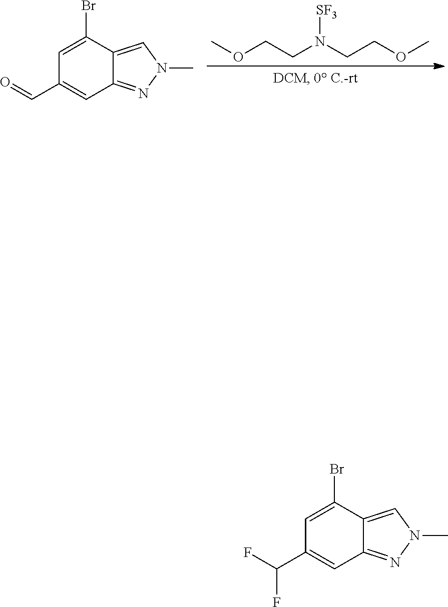

4-Bromo-6-(difluoromethyl)-2-methyl-indazole: To a solution of 4-bromo-2-methyl-indazole-6-carbaldehyde (199.3 mg, 0.83 mmol) in DCM (4 mL) at 0° C., was added Deoxo-Fluor (0.61 mL, 3.33 mmol). Methanol (10.0 μL, 0.25 mmol) was added. After 1 h, the reaction was slowly warmed up to rt. After 24 h, the reaction was quenched by sat. NaHCO₃(5 mL) and extracted with DCM (3×). The combined organic extracts were dried (Na₂SO₄), filtered, and concentrated to dryness. The crude product was then purified using normal phase chromatography on silica gel (0-100% EtOAc/hexanes) to give the title compound (83 mg, 38% yield). ¹H NMR (400 MHz, CDCl₃) δ 7.98 (s, 1H), 7.80-7.74 (m, 1H), 7.40 (d, J=1.1 Hz, 1H), 6.68 (t, J=56.1 Hz, 1H), 4.26 (s, 3H); ES-MS [M+H]⁺=261.0

The compound(s) shown in Table 17 may be prepared similarly to the compound described above, with appropriate starting materials.

TABLE 17

| Structure | Name | ¹H-NMR and/or ES-MS [M + H]⁺ |
|---|---|---|
| 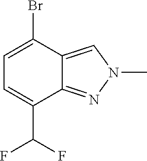 | 4-bromo-7-(difluoromethyl)-2- methyl-2H-indazole | ES-MS [M + H]⁺ = 261.0/263.0 |

### Intermediate Example 20. 4-Bromo-7-methoxy-2-methyl-2H-pyrazolo[3,4-c]pyridine

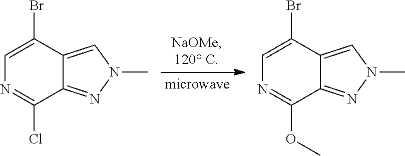

A mixture of 4-bromo-7-chloro-2-methyl-2H-pyrazolo[3,4-c]pyridine (35 mg, 0.14 mmol) and a solution of sodium methoxide (2.0 mL) was subjected to a microwave reactor at 120° C. After 30 min, the reaction mixture was concentrated under reduced pressure. The crude material was purified using normal phase chromatography on silica gel (0-100% EtOAc/hexanes) to give the title compound (28 mg, 81% yield). ¹H NMR (400 MHz, DMSO) δ 8.47 (s, 1H), 7.70 (s, 1H), 4.20 (s, 3H), 4.00 (s, 3H); ES-MS [M+H]⁺=242.0/244.0.

### Intermediate Example 21. 4-Bromo-7-cyclopropyl-2-methyl-2H-indazole

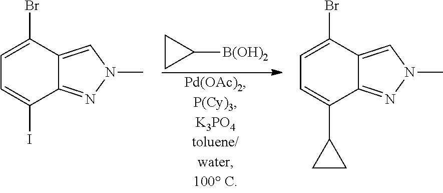

4-Bromo-7-iodo-2-methyl-2H-indazole (168 mg, 0.50 mmol), cyclopropylboronic acid (55.8 mg, 0.65 mmol), palladium acetate (22.6 mg, 0.1 mmol), tricyclohexylphosphine (56.1 mg, 0.2 mmol) and potassium phosphate tribasic (403.7 mg, 1.8 mmol) were combined in toluene (4.5 mL) and water (1.0 mL). The resulting mixture was evacuated and purged with nitrogen (3×) and stirred at 100° C. After 16 h, the reaction mixture was diluted with EtOAc and filtered through a pad of Celite®, which was washed thoroughly with EtOAc. The filtrate was concentrated and the crude material was purified using normal phase chromatography on silica gel (0-100% EtOAc/hexanes) to give the title compound (64 mg, 42% yield). ¹H NMR (400 MHz, DMSO) δ 8.34 (s, 1H), 7.12 (d, J=7.4 Hz, 1H), 6.72 (dd, J=7.5, 0.6 Hz, 1H), 4.19 (s, 3H), 2.33 (tt, J=8.3, 5.4 Hz, 1H), 0.99-0.93 (m, 4H); ES-MS [M+H]⁺=251.0/253.0.

### Intermediate Example 22. Preparation of (4,4,5,5-tetramethyl-1,3,2-dioxaborolan-2-yl)-indazoles

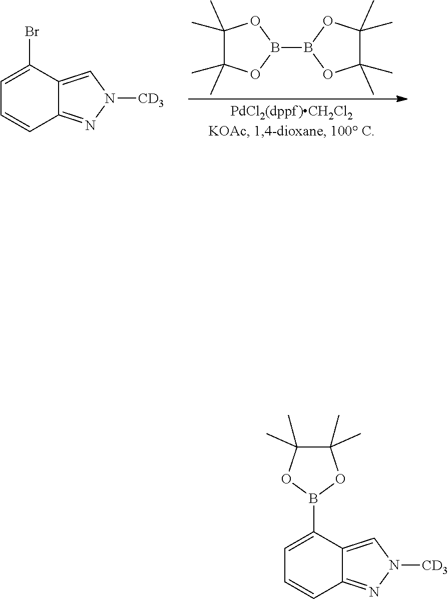

2-(Methyl-d₃)-4-(4,4,5,5-tetramethyl-1,3,2-dioxaborolan-2-yl)-2H-indazole. An oven-dried round bottom flask was charged with potassium acetate (3.31 g, 33.77 mmol, 2.5 eq.), [1,1′-bis(diphenylphosphino)ferrocene]dichloropalladium(II) complex with dichloromethane (1.66 g, 2.0 mmol, 0.15 eq.) and bis(pinocolato)diboron (5.15 g, 20.3 mmol, 1.5 eq.). After evacuating and purging with N2 (3×), a solution of 4-bromo-2-(methyl-d₃)-2H-indazole (2.89 g, 13.51 mmol, 1.0 eq.) was added. The reaction mixture was stirred at 90° C. After 16 h, the reaction mixture was diluted with EtOAc and washed with water and brine. The organic layer was dried over Na₂SO₄, filtered, and concentrated under reduced pressure. Purification using normal phase chromatography on silica gel (0-100% EtOAc/hexanes) provided the title compound (3.23 g, 91% yield). ¹H NMR (400 MHz, CDCl₃) δ 8.24 (s, 1H), 7.80 (d, J=8.7 Hz, 1H), 7.62 (d, J=6.5 Hz, 1H), 7.29 (dd, J=8.7, 6.6 Hz, 1H), 1.38 (s, 12H); ES-MS [M+H]⁺=262.2 and [boronic acid]=184.0.

### Intermediate Example 23. Preparation of 4-benzyl-6,7-dihydro-5H-pyrrolo[3,4-b]pyridin-5-ones (Reductive Amination Route)

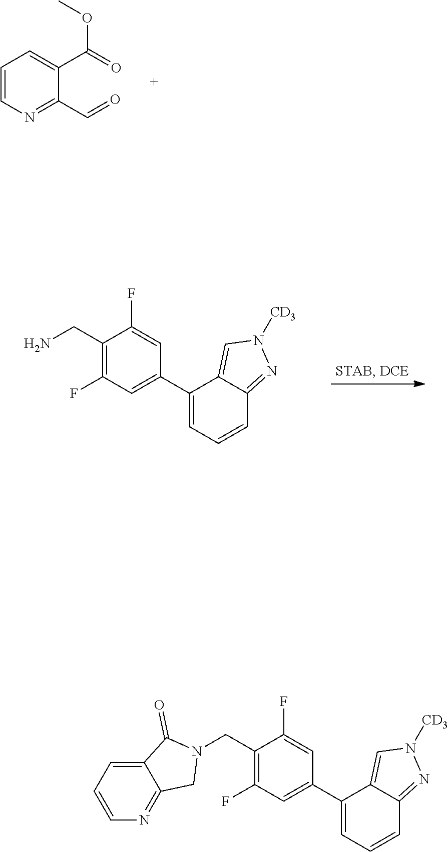 [SMILES: COC(=O)c1cccnc1C=O ; [2H]C([2H])([2H])n1cc2c(-c3cc(F)c(CN)c(F)c3)cccc2n1 ; [2H]C([2H])([2H])n1cc2c(-c3cc(F)c(CN4Cc5ncccc5C4=O)c(F)c3)cccc2n1]

6-(2,6-Difluoro-4-(2-(methyl-d₃)-2H-indazol-4-yl)benzyl)-6,7-dihydro-5H-pyrrolo[3,4-b]pyridin-5-one. A suspension of methyl 2-formylnicotinate (82.6 mg, 0.5 mmol, 1.0 eq.) and (2,6-difluoro-4-(2-(methyl-d₃)-2H-indazol-4-yl)phenyl)methanamine dihydrochloride salt (208 mg, 0.525 mmol, 1.05 eq.) in DCE (5 mL, 0.1 M) was stirred for 10 min. Sodium triacetoxyborohydride (159 mg, 0.75 mmol, 1.5 eq) was added. After 16 h at rt, the reaction mixture was diluted with DCM and washed with a sat. solution of NaHCO₃ and brine. The organic layer was dried over Na₂SO₄, filtered and concentrated. The crude material was purified using normal phase chromatography on silica gel (0-10% MeOH/DCM) to provide the title compound (84 mg, 43% yield). ¹H NMR (400 MHz, DMSO-d₆) δ 8.76 (dd, J=4.9, 1.6 Hz, 1H), 8.63 (d, J=1.0 Hz, 1H), 8.13 (dd, J=7.7, 1.6 Hz, 1H), 7.66 (dt, J=8.5, 1.0 Hz, 1H), 7.57-7.43 (m, 3H), 7.33 (dd, J=8.5, 7.0 Hz, 1H), 7.27 (dd, J=7.0, 0.9 Hz, 1H), 4.92 (s, 2H), 4.50 (s, 2H); ES-MS [M+H]⁺=394.0.

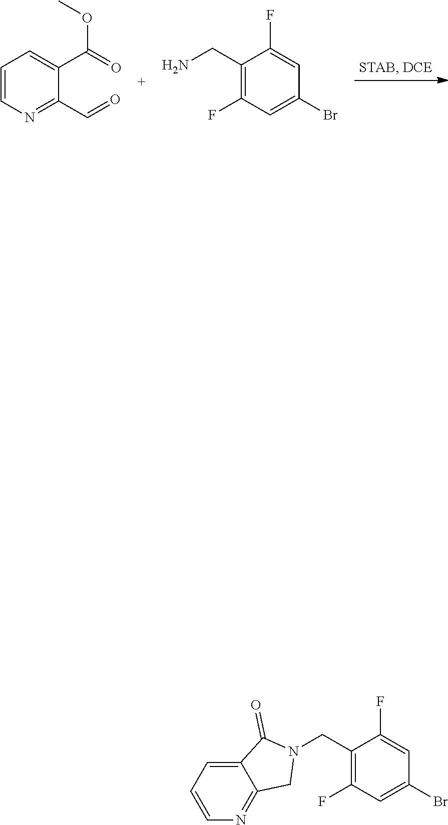 [SMILES: COC(=O)c1cccnc1C=O ; NCc1c(F)cc(Br)cc1F ; O=C1c2cccnc2CN1Cc1c(F)cc(Br)cc1F]

6-(4-Bromo-2,6-difluorobenzyl)-6,7-dihydro-5H-pyrrolo[3,4-b]pyridin-5-one was prepared in a similar manner as 6-(2,6-difluoro-4-(2-(methyl-d₃)-2H-indazol-4-yl)benzyl)-6,7-dihydro-5H-pyrrolo[3,4-b]pyridin-5-one above. ¹H NMR (400 MHz, DMSO-d₆) δ 8.74 (dd, J=4.9, 1.6 Hz, 1H), 8.10 (dd, J=7.7, 1.6 Hz, 1H), 7.57-7.47 (m, 3H), 4.81 (2H), 4.44 (s, 2H); ES-MS [M+H]⁺=339.2/341.2.

### Intermediate Example 24. Preparation of 4-benzyl-6,7-dihydro-5H-pyrrolo[3,4-b]pyridin-5-ones (Suzuki Route)

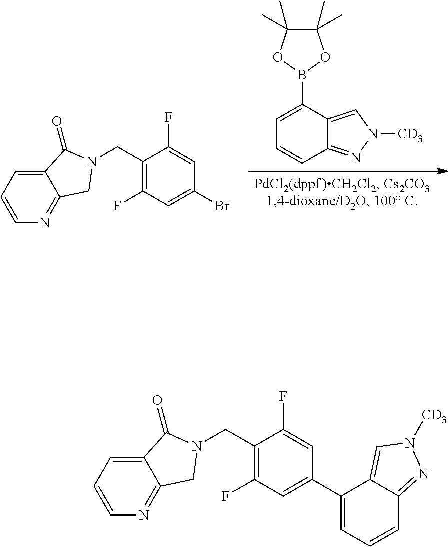

6-(2,6-Difluoro-4-(2-(methyl-d₃)-2H-indazol-4-yl)benzyl)-6,7-dihydro-5H-pyrrolo[3,4-b]pyridin-5-one can also be prepared in a similar manner as Compound 31. ¹H NMR (400 MHz, DMSO-d₆) δ 8.76 (dd, J=4.9, 1.6 Hz, 1H), 8.63 (d, J=1.0 Hz, 1H), 8.13 (dd, J=7.7, 1.6 Hz, 1H), 7.66 (dt, J=8.5, 1.0 Hz, 1H), 7.57-7.43 (m, 3H), 7.33 (dd, J=8.5, 7.0 Hz, 1H), 7.27 (dd, J=7.0, 0.9 Hz, 1H), 4.92 (s, 2H), 4.50 (s, 2H); ES-MS [M+H]⁺=394.0.

### Intermediate Example 25. Preparation of (2-methyl-2H-indazol-4-yl)phenyl)methanamines

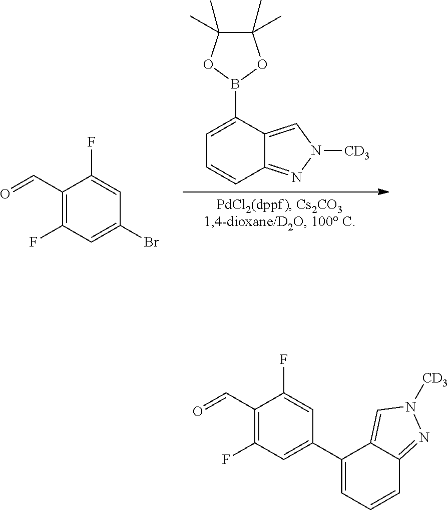

2,6-Difluoro-4-(2-(methyl-d₃)-2H-indazol-4-yl)benzaldehyde. 4-Bromo-2,6-difluorobenzaldehyde (884 mg, 4 mmol, 1.0 eq.), 2-(methyl-d₃)-4-(4,4,5,5-tetramethyl-1,3,2-dioxaborolan-2-yl)-2H-indazole (1.253 g, 4.8 mmol, 1.2 eq.), [1,1′-bis(diphenylphosphino)ferrocene]dichloropalladium(II) (220 mg, 0.3 mmol, 0.075 eq.), and cesium carbonate (2.61 g, 8.0 mmol, 2.0 eq.) were combined. Degassed 1,4-dioxane (16 mL) and H₂O (4 μL) were added. The reaction mixture was evacuated and purged with N2 (3×) and allowed to stir at 100° C. After 1 h, the reaction mixture was diluted with 10% MeOH/DCM solution. The layers were separated, and the aqueous layer was extracted with 10% MeOH/DCM (3×). The combined extracts were dried over Na₂SO₄, filtered and concentrated. The crude material was suspended in EtOAc. The precipitate was collected using vacuum filtration, washed with EtOAc and dried under vacuum to provide the tittle compound, which was used in the subsequence step without further purification (1.53 g). ¹H NMR (400 MHz, DMSO-d₆) δ 10.26 (s, 1H), 8.70 (s, 1H), 7.74 (d, J=8.1 Hz, 1H), 7.63 (d, J=10.3 Hz, 2H), 7.44-7.37 (m, 1H), 7.38 (d, J=7.9 Hz, 1H); ES-MS [M+H]⁺=276.0.

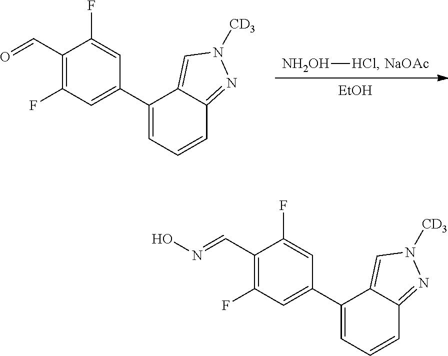

(E/Z)-2,6-Difluoro-4-(2-(methyl-d₃)-2H-indazol-4-yl)benzaldehyde oxime. To a suspension of 2,6-difluoro-4-(2-(methyl-d₃)-2H-indazol-4-yl)benzaldehyde (1.53 g, 4.0 mmol, 1.0 eq.) in ethanol (20 mL, 0.2 M) was added sodium acetate (655 mg, 8.0 mmol, 2.0 eq.) and hydroxylamine hydrochloride (555 mg, 8.0 mmol, 2.0 eq.). After 1 h at rt, the reaction mixture was diluted with EtOAc, washed with H₂O and brine. The organic layer was dried over Na₂SO₄, filtered and concentrated to provide the title compound, which was carried forward without further purification (1.18 g). ¹H NMR (400 MHz, DMSO-d₆) δ 8.66 (d, J=1.0 Hz, 1H), 8.21 (s, 1H), 7.68 (dt, J=7.8, 1.3 Hz, 1H), 7.59-7.46 (m, 2H), 7.41-7.26 (m, 2H), OH proton not observable; ES-MS [M+H]⁺=291.0.

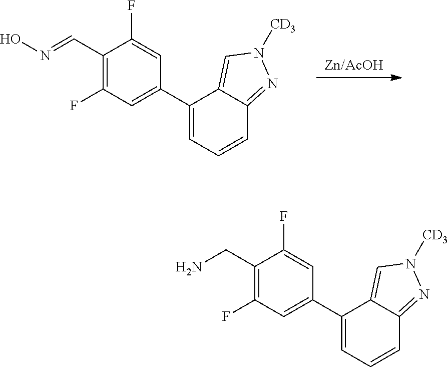 [SMILES: [2H]C([2H])([2H])n1cc2c(-c3cc(F)c(/C=N/O)c(F)c3)cccc2n1 ; "Zn/AcOH" ; [2H]C([2H])([2H])n1cc2c(-c3cc(F)c(CN)c(F)c3)cccc2n1]

(2,6-Difluoro-4-(2-(methyl-d₃)-2H-indazol-4-yl)phenyl)methanamine. To (E/Z)-2,6-difluoro-4-(2-(methyl-d₃)-2H-indazol-4-yl)benzaldehyde oxime (1.18 g, 3.98 mmol, 1.0 eq.) in acetic acid (20 mL) was added zinc powder (1.3 g, 19.92 mmol, 5.0 eq.). The mixture was sonicated at rt for 2 h then filtered through a pad of Celite® which was washed thoroughly with 10% MeOH/DCM. The filtrate was concentrated under reduced pressure. The crude green oil was re-dissolved in 10% MeOH/DCM and filtered through a phase separator to remove any insoluble salt and concentrated. The resulting residue was dissolved in 1,4-dioxane (5.0 mL). A solution of 4 M HCl in 1,4-dioxane (4.98 mL, 19.92 mmol, 5.0 eq.) was added and sonicated for 10-15 min. The precipitate was collected using vacuum filtration, washed with 1,4-dioxane (5.0 mL) and dried under vacuum to provide the title compound as a HCl salt (1.6 g, 88% purity). ¹H NMR (400 MHz, DMSO-d₆) δ 8.61 (d, J=1.0 Hz, 1H), 8.41 (bs, 2H), 7.69 (d, J=8.3 Hz, 1H), 7.63-7.54 (m, 2H), 7.35 (dd, J=8.3, 7.0 Hz, 1H), 7.31 (dd, J=7.0, 1.1 Hz, 1H), 4.14 (dd, J=5.8 Hz, 2H); ES-MS [M+H]⁺=277.0.

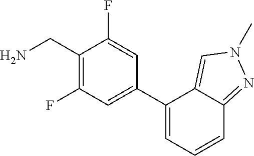

(2,6-Difluoro-4-(2-methyl-2H-indazol-4-yl)phenyl)methanamine was prepared in a similar manner as (2,6-difluoro-4-(2-(methyl-d₃)-2H-indazol-4-yl)phenyl)methanamine using 2-methyl-4-(4,4,5,5-tetramethyl-1,3,2-dioxaborolan-2-yl)-2H-indazole. ¹H NMR (400 MHz, DMSO) δ 8.62 (s, 1H), 7.68 (d, J=8.3 Hz, 1H), 7.62-7.52 (m, 2H), 7.35 (dd, J=8.3, 7.0 Hz, 1H), 7.30 (dd, J=7.0, 1.1 Hz, 1H), 4.66 (bs, 2H), 4.21 (s, 3H), 4.11 (dd, J=5.8 Hz, 2H); ES-MS [M+H]⁺=274.0.

### Intermediate Example 26. Preparation of (4-Bromopyrazolo[1,5-a]pyridin-2-yl)methanol

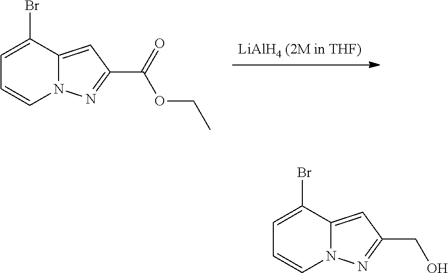

To a solution of ethyl 4-bromopyrazolo[1,5-a]pyridine-2-carboxylate (80 mg, 0.3 mmol) in THF at 0° C., LiAlH₄ (2M in THF, 0.15 mL, 0.3 mmol) was added. The reaction mixture was stirred at 0° C. After 20 min at 0° C., to the reaction mixture was added H₂O (1 mL) dropwise. The ice bath was removed and the reaction mixture was extracted with DCM (3×10 mL). The combined organics were passed through a phase separator and concentrated in vacuo. The residue (83 mg) was used for the next step without further purification. ¹H-NMR (400 MHz, CDCl₃) δ 8.27 (d, J=7.0 Hz, 1H), 7.23 (d, J=7.2 Hz, 1H), 6.56-6.51 (m, 2H), 4.84 (s, 2H), 3.90 (br s, 1H). ES-MS [M+H]⁺=227 and 229.

### Intermediate Example 27. Preparation of (4-Bromopyrazolo[1,5-a]pyridin-2-yl)methan-d₂-ol

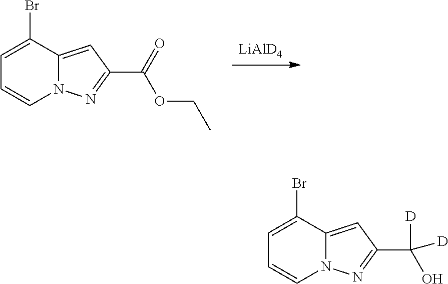

The title compound was synthesized analogously to Intermediate Example 26, using ethyl 4-bromopyrazolo[1,5-a]pyridine-2-carboxylate (50 mg, 0.19 mmol) and LiAlD₄ (14.1 mg, 0.37 mmol). 34.7 mg (81% yield) was obtained. ES-MS [M+H]⁺=229 and 231.

TABLE 18
Commercial Starting Materials

| Name | CAS# | Supplier |
|---|---|---|
| furo[3,4-b]pyridine-5,7-dione | 699-98-9 | Ambeed |
| (4-bromo-2,6-difluorophenyl)meth- | 1461655- | Enamine |
| anamine | 72-0 (HCl) | |
| (4-bromo-2-fluorophenyl)methanamine | 112734-22-2 | Combi-Blocks |
| (4-bromo-2-methylphenyl)methanamine | 376646-62-7 | Ambeed |
| (4-bromophenyl)methanamine | 3959-07-7 | Combi-Blocks |
| 4,4,4′,4′,5,5,5′,5′-octamethyl-2,2′- | 73183-34-3 | Combi-Blocks |
| bi(1,3,2-dioxaborolane) | | |
| 2-methyl-4-(4,4,5,5-tetramethyl-1,3,2- | 885698-95-3 | Combi-Blocks |
| dioxaborolan-2-yl)-2H-indazole | | |
| 4-chloro-2-methyl-2H-pyrazolo[3,4- | 944501-84-2 | Aurum |
| b]pyridine | | Pharmatech |
| 5-bromo-2-methyl-2H-pyrazolo[3,4- | 1316852-65-9 | Synthonix |
| b]pyridine | | |
| 5-bromo-6-fluoro-2-methyl-2H-indazole | 2091272-22-7 | Aurum |
| | | Pharmatech |
| 7-bromoimidazo[1,2-a]pyridine | 808744-34-5 | Combi-Blocks |
| 5-bromo-2,6-dimethyl-2H-indazole | 1159511-90-6 | Bellen |
| | | Chemistry |
| 5-bromo-7-fluoro-2-methyl-2H-indazole | 2004705-97-7 | Enamine |
| 1-methyl-5-(4,4,5,5-tetramethyl-1,3,2- | 1235469-00-7 | Combi-Blocks, |
| dioxaborolan-2-y1)-1H-indazole | | Inc. |
| 2-methyl-6-(4,4,5,5-tetramethyl-1,3,2- | 1204580-79-9 | Combi-Blocks |
| dioxaborolan-2-y1)-2H-indazole | | |
| 5-bromo-2,7-dimethyl-2H-indazole | 1146637-10-6 | Combi-Blocks |
| 6-bromoimidazo[1,5-a]pyridine | 1239880-00-2 | WuXi AppTec |
| 4-bromo-2,7-dimethyl-2H-indazole | 1159511-88-2 | BLD |
| | | Pharmatech |
| 4-bromo-2,6-dimethyl-2H-indazole | 1159511-87-1 | AOBChem |
| 4-bromo-6-fluoro-2-methyl-2H-indazole | 1425932-77-9 | ChemSpace |
| 4-bromo-1H-indazole | 186407-74-9 | Ambeed |
| 4-bromo-7-methyl-1H-indazole | 1159511-74-6 | Aurum |
| | | Pharmatech |
| 4-bromo-7-fluoro-1H-indazole | 1186334-63-3 | BLD |
| | | Pharmatech |
| 4-bromo-5-fluoro-1H-indazole | 1056264-22-2 | BLD |
| | | Pharmatech |
| 4-bromo-7-methoxy-1H-indazole | 938062-01-2 | AstaTech |
| 4-bromo-7-chloro-1H-indazole | 1186334-61-1 | Ambeed |
| 4-bromo-7-(trifluoromethyl)-1H- | 1186334-79-1 | BLD |
| indazole | | Pharmatech |
| methyl 2-formylnicotinate | 25230-59-5 | Enamine |
| 4-bromo-2,6-difluorobenzaldehyde | 537013-51-7 | BLD |
| | | Pharmatech |
| 3-bromofuro[3,4-b]pyridine-5,7-dione | 98278-78-5 | Combi-Blocks, |
| | | Inc. |
| ethyl 5-bromo-2-methylnicotinate | 129477-21-0 | AmBeed |
| 1,3,5-trichloro-1,3,5-triazinane-2,4,6- | 87-90-1 | Sigma-Aldrich |
| trione | | |
| 4-bromo-6-chloro-1H-indazole | 885519-03-9 | Combi-Blocks |
| 5-bromo-7-methoxy-1H-indazole | 1374652-62-6 | AstaTech |
| 4-bromo-7-chloro-6-fluoro-1H-indazole | 1935558-98-7 | Ambeed |
| 4-bromo-2-fluoro-6- | 3261644-43-9 | Enamine |
| methylphenyl)methanamine | | |
| hydrochloride | | |
| 4-bromo-6-methyl-1H-pyrazolo[3,4- | 1446236-51-6 | AstaTech |
| c]pyridine-7-one | | |
| methyl 4-bromo-2-methylindazole-6- | 1638772-21-0 | AstaTech |
| carboxylate | | |
| 4-bromo-2-chloro-6-fluorobenzaldehyde | 929621-33-0 | |
| 6-bromo-2-fluoro-3-hydroxy- | 935534-46-6 | ABOChem |
| benzaldehyde | | |
| 4-bromo-2-fluoro-6- | 2090805-34-6 | Enamine |
| (trifluoromethyl)benzaldehyde | | |
| 4-bromo-2,6-dichlorobenzaldehyde | 111829-72-2 | Ambeed |
| 5-bromo-7-chloroindazole | 635712-44-6 | J &W |
| | | PharmLab |
| 6-bromo-2-fluoro-3- | 524674-70-2 | Asta Tech |
| (trifluoromethoxy)benzaldehyde | | |
| 4-bromo-2-fluoro-6-methoxy- | 856767-09-4 | Ambeed |
| benzaldehyde | | |
| 4-bromo-2-isopropylbenzaldehyde | 1114808-82-0 | Aurum |
| | | Pharmatech |
| 6-bromo-2,3,4-trifluorobenzaldehyde | 372519-10-3 | Enamine |
| 4-bromo-2,6-difluorobenzoic acid | 183065-68-1 | Sigma-Aldrich |
| 4-bromo-2-chloro-6-fluorobenzoic acid | 1321613-01-7 | Ambeed |
| 4-bromo-1H-pyrazolo[3,4-c]pyridine | 1032943-43-3 | Ambeed |
| 4-bromo-7-chloro-1H-pyrazolo[3,4- | 1446222-51-0 | Ambeed |
| c]pyridine | | |
| 4-bromo-7-iodo-1H-indazole | 1449008-23-4 | Frontier |
| | | Scientific |
| 4-chloro-1H-pyrazolo[3,4-b]pyridine | 29274-28-0 | Ambeed |
| 4-bromo-7-methoxy-1 H -indazole | 938062-01-2 | BLDPharm |
| 1-bromo-4-fluoro-2-methyl-3-nitro- | 1227210-35-6 | Enamine |
| benzene | | |
| ethyl 4-bromopyrazolo[1,5-a]pyridine- | 1363381-99-0 | AA Block |
| 2-carboxylate | | |

### Preparation of Representative Compounds

### Example 1. Synthetic Route A

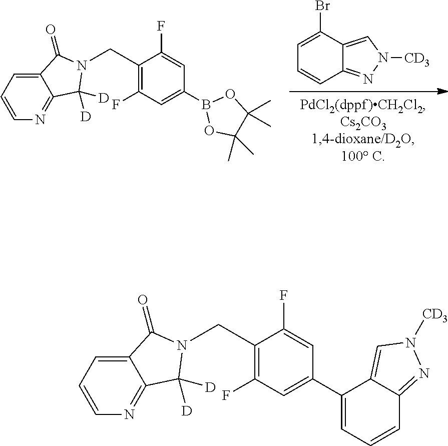 [SMILES: [2H]C1([2H])c2ncccc2C(=O)N1Cc1c(F)cc(B2OC(C)(C)C(C)(C)O2)cc1F ; [2H]C([2H])([2H])n1cc2c(Br)cccc2n1 ; "PdCl2(dppf)·CH2Cl2,Cs2CO31,4-dioxane/D2O,100° C." ; [2H]C1([2H])c2ncccc2C(=O)N1Cc1c(F)cc(-c2cccc3nn(C([2H])([2H])[2H])cc23)cc1F]

6-(2,6-Difluoro-4-(2-(methyl-d₃)-2H-indazol-4-yl)benzyl)-6,7-dihydro-5H-pyrrolo[3,4-b]pyridin-5-one-7,7-d₂ (Compound 31). To a solution of 6-(2,6-difluoro-4-(4,4, 5, 5-tetramethyl-1,3,2-dioxaborolan-2-yl)benzyl)-6,7-dihydro-5H-pyrrolo[3,4-b]pyridin-5-one-7,7-d₂ (1.55 g, 4.0 mmol, 1.0 eq) in degassed 1,4-dioxane (18 mL, 0.2 M) were added [1,1′-bis(diphenylphosphino)ferrocene]dichloropalladium(II) (367 mg, 0.5 mmol, 0.125 eq), cesium carbonate (3.93 g, 12 mmol, 3.0 eq), 4-bromo-2-(methyl-d₃)-2H-indazole (1.55 g, 4.0 mmol, 1.0 eq) and D₂O (1.8 mL). The reaction mixture was evacuated and purged with N2 (3×) and allowed to stir at 100° C. After 1 h, the mixture was diluted with EtOAc and DCM and filtered through a pad of Celite®. The filtrate was concentrated under reduced pressure. The crude material was purified using normal phase chromatography on silica gel (0-50% of 10% MeOH/DCM solution) then reverse phase HPLC (20-60% MeCN in 0.05% NH₄₀H aqueous solution) to provide the title compound (855 mg, 55% yield, 92% deuterium incorporated by HRMS and ¹H NMR). ¹H NMR (400 MHz, DMSO-d₆) δ 8.75 (dd, J=5.0, 2.2 Hz, 1H), 8.62 (d, J=0.8 Hz, 1H), 8.12 (dd, J=7.7, 3.1 Hz, 1H), 7.66 (d, J=8.5 Hz, 1H), 7.46-7.55 (m, 3H), 7.33 (dd, J=8.5, 5.2, 1H), 7.27 (d, J=3.4, 1H), 4.91 (s, 2H); ES-MS [M+H]⁺=396.0.

### Example 2. Synthetic Route B

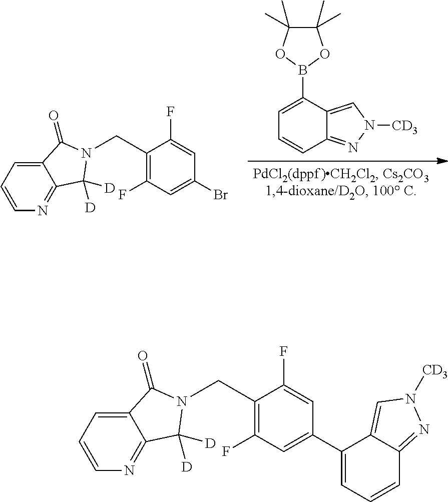

6-(2,6-Difluoro-4-(2-(methyl-d₃)-2H-indazol-4-yl)benzyl)-6,7-dihydro-5H-pyrrolo[3,4-b]pyridin-5-one-7,7-d₂ (Compound 31). 6-(4-Bromo-2,6-difluorobenzyl)-6,7-dihydro-5H-pyrrolo[3,4-b]pyridin-5-one-7,7-d₂ (34.1 mg, 0.1 mmol, 1.0 eq.), 2-(methyl-d₃)-4-(4,4,5,5-tetramethyl-1,3,2-dioxaborolan-2-yl)-2H-indazole (39.2 mg, 0.15 mmol, 1.5 eq.), [1,1′-bis(diphenylphosphino)ferrocene]dichloropalladium(II) (11 mg, 0.015 mmol, 0.15 eq.), cesium carbonate (65.6 mg, 0.2 mmol, 3.0 eq.) were combined. Degassed 1,4-dioxane (0.5 mL) and D₂O (50 μL) were added. The reaction mixture was evacuated and purged with N2 (3×) and allowed to stir at 100° C. After 1 h, the mixture was diluted with EtOAc and DCM and filtered through a pad of Celite®. The filtrate was concentrated under reduced pressure. The crude material was purified by reverse phase HPLC (20-60% MeCN in 0.1% TFA aqueous solution) to provide the title compound (20 mg, 51% yield, 94% deuterium incorporated by HRMS and ¹H NMR). ¹H NMR (400 MHz, DMSO-d₆) δ 8.75 (dd, J=5.0, 2.2 Hz, 1H), 8.62 (d, J=0.8 Hz, 1H), 8.12 (dd, J=7.7, 3.1 Hz, 1H), 7.66 (d, J=8.5 Hz, 1H), 7.46-7.55 (m, 3H), 7.33 (dd, J=8.5, 5.2, 1H), 7.27 (d, J=3.4, 1H), 4.91 (s, 2H); ES-MS [M+H]⁺=396.0.

### Example 3. Synthetic Route C

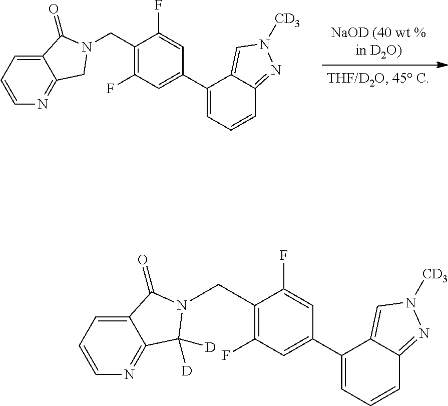 [SMILES: [2H]C([2H])([2H])n1cc2c(-c3cc(F)c(CN4Cc5ncccc5C4=O)c(F)c3)cccc2n1 ; "NaOD (40 wt %in D2O)" ; "THF/D2O, 45° C." ; [2H]C1([2H])c2ncccc2C(=O)N1Cc1c(F)cc(-c2cccc3nn(C([2H])([2H])[2H])cc23)cc1F]

6-(2,6-Difluoro-4-(2-(methyl-d₃)-2H-indazol-4-yl)benzyl)-6,7-dihydro-5H-pyrrolo[3,4-b]pyridin-5-one-7,7-d₂ (Compound 31). To a suspension of 6-(2,6-difluoro-4-(2-(methyl-d₃)-2H-indazol-4-yl)benzyl)-6,7-dihydro-5H-pyrrolo[3,4-b]pyridin-5-one (11.0 mg, 0.03 mmol, 1.0 eq.) in THE (0.2 mL) was added D₂O (0.2 mL) followed by a solution of sodium deuteroxide (40 wt % in D₂O, 10 μL, 0.14 mmol, 5.0 eq.). The reaction was stirred at 45° C. After 48 h, the reaction mixture was diluted with DCM, filtered through a phase separator and concentrated. Purification using reverse phase HPLC (20-60% MeCN in 0.1% TFA aqueous solution) provided the title compound (6.0 mg, 54% yield, >98% deuterium incorporation by HRMS and ¹H NMR). ¹H NMR (400 MHz, DMSO-d₆) δ 8.75 (dd, J=5.0, 2.2 Hz, 1H), 8.62 (d, J=0.8 Hz, 1H), 8.12 (dd, J=7.7, 3.1 Hz, 1H), 7.66 (d, J=8.5 Hz, 1H), 7.55-7.46 (m, 3H), 7.33 (dd, J=8.5, 5.2, 1H), 7.27 (d, J=3.4, 1H), 4.91 (s, 2H); ES-MS [M+H]⁺=396.0.

### Example 4. 3-Bromo-6-(2,6-difluoro-4-(2-methyl-2H-indazol-4-yl)benzyl)-6,7-dihydro-5H-pyrrolo[3,4-b]pyridin-5-one-7,7-d₂ (Compound 61)

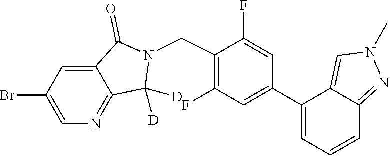

The title compound was prepared in a similar manner as Intermediate 3 above from 3-bromo-6-(2,6-difluoro-4-(2-methyl-2H-indazol-4-yl)benzyl)-7-hydroxy-6,7-dihydro-5H-pyrrolo[3,4-b]pyridin-5-one-7-d. ¹H NMR (400 MHz, DMSO-d₆) δ 8.91 (d, J=2.2 Hz, 1H), 8.63 (d, J=0.9 Hz, 1H), 8.37 (d, J=2.2 Hz, 1H), 7.67 (d, J=8.5 Hz, 1H), 7.53-7.47 (m, 2H), 7.34 (dd, J=8.5, 7.0 Hz, 1H), 7.28 (dd, J=6.9, 0.9 Hz, 1H), 4.91 (s, 2H), 4.19 (s, 3H); ES-MS [M+H]⁺=473.2.

### Example 5. 6-(2,6-Difluoro-4-(2-methyl-2H-indazol-4-yl)benzyl)-3-hydroxy-6,7-dihydro-5H-pyrrolo[3,4-b]pyridin-5-one-7,7-d₂ (Compound 62)

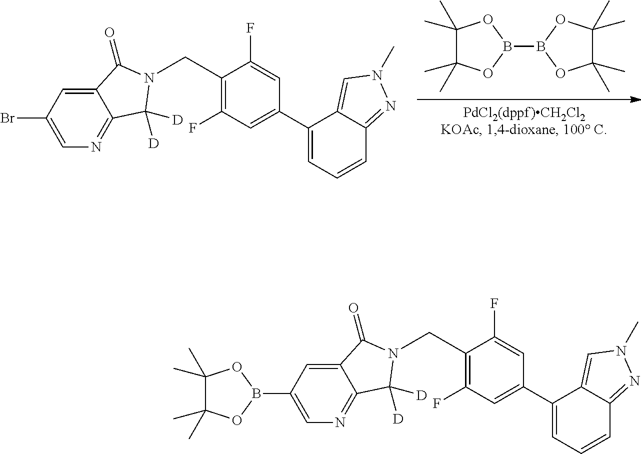

6-(2,6-Difluoro-4-(2-methyl-2H-indazol-4-yl)benzyl)-3-(4,4,5,5-tetramethyl-1,3,2-dioxaborolan-2-yl)-6,7-dihydro-5H-pyrrolo[3,4-b]pyridin-5-one-7,7-d₂ was prepared in a similar manner as 2-(methyl-d₃)-4-(4,4,5,5-tetramethyl-1,3,2-dioxaborolan-2-yl)-2H-indazole (Intermediate 4). ES-MS [boronic acid]=437.0.

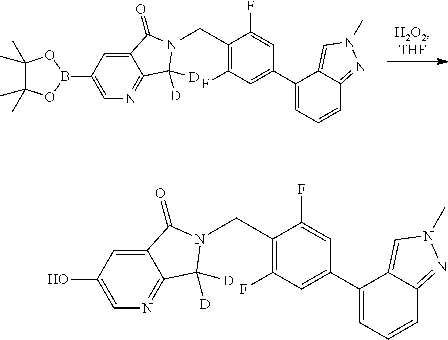

6-(2,6-Difluoro-4-(2-methyl-2H-indazol-4-yl)benzyl)-3-hydroxy-6,7-dihydro-5H-pyrrolo[3,4-b]pyridin-5-one-7,7-d₂ (Compound 62). To a solution of 6-(2,6-difluoro-4-(2-methyl-2H-indazol-4-yl)benzyl)-3-(4,4,5,5-tetramethyl-1,3,2-dioxaborolan-2-yl)-6,7-dihydro-5H-pyrrolo[3,4-b]pyridin-5-one-7,7-d₂ (13.3 mg, 0.03 mmol, 1.0 eq.) in THE (0.5 mL) was added hydrogen peroxide solution (30% in water, 5 μL, 0.15 mmol, 5.0 eq.). After 2 h at rt, water was added and the mixture was concentrated under reduced pressure. Purification using RP-HPLC provided the title compound. ES-MS [M+H]⁺=409.3.

### Example 6. 6-(2,6-Difluoro-4-(2-methyl-2H-indazol-4-yl)benzyl)-3-(methoxy-d₃)-6,7-dihydro-5H-pyrrolo[3,4-b]pyridin-5-one-7,7-d₂ (Compound 63)

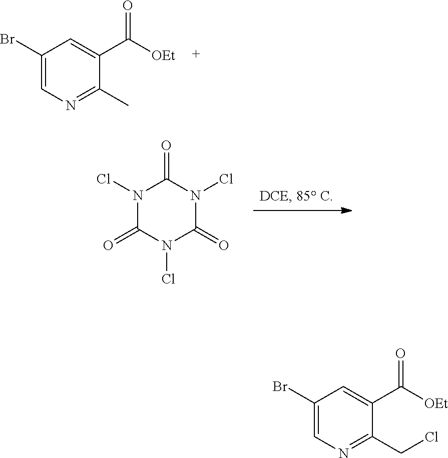

Ethyl 5-bromo-2-(chloromethyl)nicotinate. A suspension of ethyl 5-bromo-2-methylnicotinate (19.53 g, 80 mmol, 1.0 eq.) and 1,3,5-trichloro-1,3,5-triazinane-2,4,6-trione (33.47 g, 144 mmol, 1.8 eq.) in DCE (400 mL) was stirred at 85° C. After 16 h, the precipitate was filtered off. The filtrate was diluted with DCM, washed sequentially with a sat. soln. of NaHCO₃, water, and brine, and concentrated under reduced pressure. Purification using normal phase chromatography on silica gel (0-10% EtOAc/hexanes) provided the title compound (13.6 g, 61% yield). ¹H NMR (400 MHz, CDCl₃) δ 8.76 (d, J=2.3 Hz, 1H), 8.40 (d, J=2.3 Hz, 1H), 5.06 (s, 2H), 4.44 (q, J=7.1 Hz, 2H), 1.44 (t, J=7.1 Hz, 3H); ES-MS [M+H]⁺=278.2/280.2.

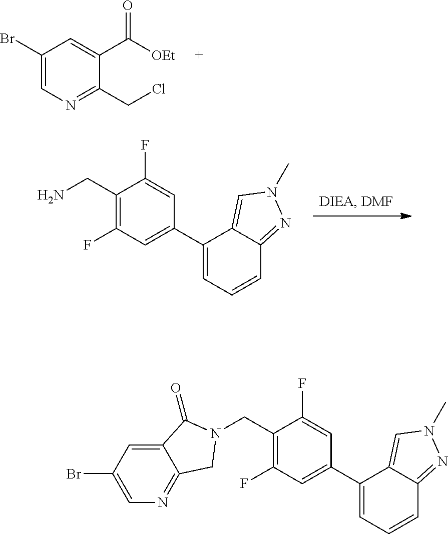

3-Bromo-6-(2,6-difluoro-4-(2-methyl-2H-indazol-4-yl)benzyl)-6,7-dihydro-5H-pyrrolo[3,4-b]pyridin-5-one. A mixture of ethyl 5-bromo-2-(chloromethyl)nicotinate (557 mg, 2.0 mmol, 1.0 eq.), (2,6-difluoro-4-(2-methyl-2H-indazol-4-yl)phenyl)methanamine (1.09 g, 4.0 mmol, 2.0 eq) and N,N-diisopropylethyl amine (0.697 mL, 4.0 mmol, 2.0 eq.) in DMF (2.0 mL) was stirred at rt. After 16 h, the precipitate was collected using vacuum filtration, washed with EtOAc and dried to provide the title compound (379 mg, 62% yield). ¹H NMR (400 MHz, CDCl₃) δ 8.77 (d, J=2.2 Hz, 1H), 8.26 (d, J=2.2 Hz, 1H), 8.07 (s, 1H), 7.78 (d, J=8.7 Hz, 1H), 7.39 (dd, J=8.7, 7.0 Hz, 1H), 7.26-7.24 (m, 2H), 7.17 (d, J=7.0 Hz, 1H), 5.00 (s, 2H), 4.42 (s, 2H), 4.28 (s, 3H); ES-MS [M+H]⁺=469.0/471.0.

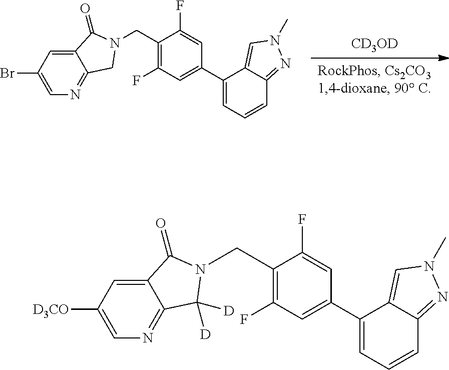

6-(2,6-Difluoro-4-(2-methyl-2H-indazol-4-yl)benzyl)-3-(methoxy-d₃)-6,7-dihydro-5H-pyrrolo[3,4-b]pyridin-5-one-7,7-d₂ (Compound 63). 3-Bromo-6-(2,6-difluoro-4-(2-methyl-2H-indazol-4-yl)benzyl)-6,7-dihydro-5H-pyrrolo[3,4-b]pyridin-5-one (141 mg, 0.3 mmol, 1.0 eq.), RockPhos Palladacycle Gen-3 (50.4 mg, 0.06 mmol, 0.2 eq.) and cesium carbonate (256 mg, 0.78 mmol, 2.6 eq.) were combined. Degassed 1,4-dioxane (3.0 mL) was added followed by methanol-d₄ (0.3 mL, 9.0 mmol, 30 eq.). After evacuation and purging with nitrogen (3×), the reaction mixture was stirred at 90° C. for 16 h and filtered through a pad of Celite®, which was rinsed with EtOAc/DCM. The filtrate was concentrated under reduced pressure. Purification using RP-HPLC provided the title compound with 74% deuterium incorporation by HRMS. The material was then dissolved in THE (0.5 mL) and deuterated water (0.5 mL). A solution of sodium deuteroxide (40% in deuterated water, 32 μL) was added. The resulting mixture was stirred at 80° C. After 3 h, DCM was added, and the layers were separated. The aqueous layer was extracted with DCM (3×) and the combined extracts were washed with brine, dried over Na₂SO₄, filtered and concentrated. Purification using normal phase chromatography on silica gel (0-10% MeOH/DCM) provided the title compound with >98% deuterium incorporation (51 mg, 41% yield). ¹H NMR (400 MHz, DMSO-d₆) δ 8.64 (d, J=1.0 Hz, 1H), 8.46 (d, J=2.8 Hz, 1H), 7.70-7.63 (m, 2H), 7.53-7.47 (m, 2H), 7.34 (dd, J=8.5, 6.9 Hz, 1H), 7.28 (dd, J=6.9, 0.9 Hz, 1H), 4.92 (s, 2H), 4.19 (s, 3H); ES-MS [M+H]⁺=426.1.

### Example 7. 6-(2,6-Difluoro-4-(7-hydroxy-2-methyl-2H-indazol-4-yl)benzyl)-6,7-dihydro-5H-pyrrolo[3,4-b]pyridin-5-one-7,7-d₂ (Compound 130)

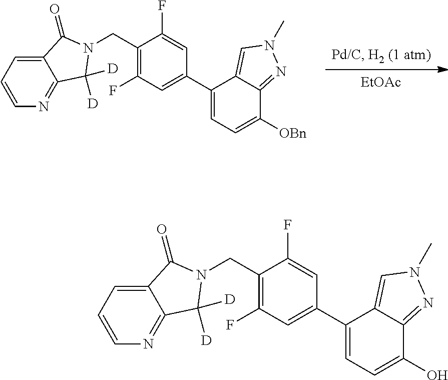 [SMILES: "EtOAc" ; [2H]C1([2H])c2ncccc2C(=O)N1Cc1c(F)cc(-c2ccc(OCc3ccccc3)c3nn(C)cc23)cc1F ; "Pd/C, H2 (1 atm)" ; [2H]C1([2H])c2ncccc2C(=O)N1Cc1c(F)cc(-c2ccc(O)c3nn(C)cc23)cc1F]

A suspension of 6-(4-(7-(benzyloxy)-2-methyl-2H-indazol-4-yl)-2,6-difluorobenzyl)-6,7-dihydro-5H-pyrrolo[3,4-b]pyridin-5-one-7,7-d₂ (23 mg, 0.046 mmol) and palladium on carbon (10 wt. % loading, matrix activated carbon support, 50 mg) in EtOAc (2.5 mL) was evacuated and purged with hydrogen (1 atm). After 16 h at rt, the mixture was filtered through a pad of Celite®, which was washed thoroughly with EtOAc. The filtrate was concentrated under reduced pressure. Purification using RP-HLPC provided the title compound. ¹H NMR (400 MHz, CDCl₃) δ 10.14 (s, 1H), 8.75 (dd, J=4.9, 1.6 Hz, 1H), 8.57 (s, 1H), 8.12 (dd, J=7.7, 1.6 Hz, 1H), 7.53 (dd, J=7.7, 4.9 Hz, 1H), 7.41-7.35 (m, 2H), 7.12 (d, J=7.6 Hz, 1H), 6.56 (d, J=7.6 Hz, 1H), 4.88 (s, 2H), 4.17 (s, 3H); ES-MS [M+H]⁺=409.0.

### Example 8. 6-(2-Cyclopropyl-6-fluoro-4-(3-fluoro-2-(methyl-d₃)-2H-indazol-4-yl)benzyl)-6,7-dihydro-5H-pyrrolo[3,4-b]pyridin-5-one-7,7-d₂ (Compound 191)

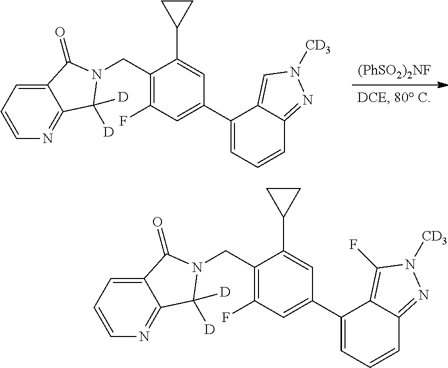

To a solution of 6-(2-cyclopropyl-6-fluoro-4-(2-(methyl-d₃)-2H-indazol-4-yl)benzyl)-6,7-dihydro-5H-pyrrolo[3,4-b]pyridin-5-one-7,7-d₂ (12 mg, 0.03 mmol) in DCE (1.0 mL) was added N-fluorobenzenesulfonimide (13.5 mg, 0.043 mmol). The resulting mixture was stirred at 80° C. After 16 h, water was added, and the reaction mixture was extracted with DCM (3×). The combined extracts were dried over Na₂SO₄, filtered, and concentrated. Purification using RP-HLPC provided the title compound. ¹H NMR (400 MHz, CDCl₃) δ 8.71 (dd, J=5.0, 1.6 Hz, 1H), 8.14 (dd, J=7.7, 1.6 Hz, 1H), 7.52 (ddd, J=8.8, 2.1, 0.7 Hz, 1H), 7.41 (dd, J=7.7, 5.0 Hz, 1H), 7.30 (dd, J=8.8, 6.9 Hz, 1H), 7.18 (dt, J=10.7, 2.0 Hz, 1H), 7.03 (d, J=6.9 Hz, 1H), 6.97 (t, J=2.4 Hz, 1H), 5.18 (d, J=1.9 Hz, 2H), 2.25 (ddd, J=13.5, 8.4, 5.2 Hz, 1H), 1.08-0.98 (m, 2H), 0.76-0.67 (m, 2H); ES-MS [M+H]⁺=436.2.

### Example 9. 6-(2,6-Difluoro-4-(2-methylpyrazolo[1,5-a]pyridin-4-yl)benzyl)-6,7-dihydro-5H-pyrrolo[3,4-b]pyridin-5-one-7,7-d₂ (Compound 235)

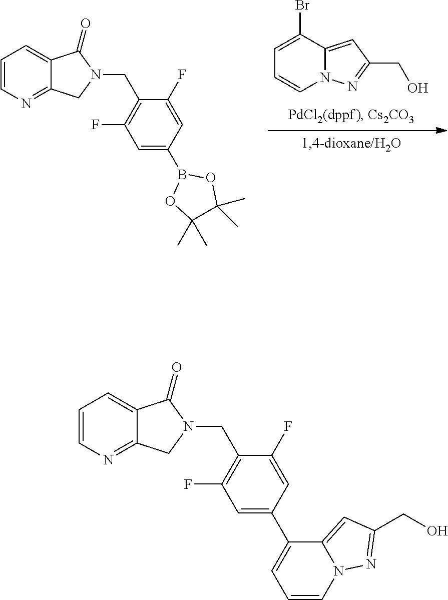

6-(2,6-Difluoro-4-(2-(hydroxymethyl)pyrazolo[1,5-a]pyridin-4-yl)benzyl)-6,7-dihydro-5H-pyrrolo[3,4-b]pyridin-5-one. (4-Bromopyrazolo[1,5-a]pyridin-2-yl)methanol (67 mg, 0.30 mmol), 6-(2,6-difluoro-4-(4,4,5,5-tetramethyl-1,3,2-dioxaborolan-2-yl)benzyl)-6,7-dihydro-5H-pyrrolo[3,4-b]pyridin-5-one (148 mg, 0.38 mmol), Cs₂CO₃ (290 mg, 0.89 mmol), and Pd(dppf)Cl₂ (32 mg, 0.04 mmol) were added to a vial. The reaction mixture was purged with nitrogen. To the reaction mixture was added a 1,4-dioxane/H₂O solution (4:1) (2.5 mL, degassed) via syringe. The resulting mixture was heated at 100° C. for 2 h. At room temperature, the reaction mixture was filtered through Celite® and the Celite® was washed with EtOAc (3×10 mL). The combined organic layers were concentrated under reduced pressure. The crude residue was purified by normal-phase column chromatography (0-10% MeOH in DCM) to afford the title compound (103 mg). ¹H-NMR (400 MHz, MeOD) δ 8.72 (d, J=5.0 Hz, 1H), 8.50 (d, J=7.0 Hz, 1H), 8.17 (d, J=7.8 Hz, 1H), 7.55 (dd, J=7.8, 5.0 Hz, 1H), 7.41 (d, J=8.3 Hz, 2H), 7.33 (d, J=7.1 Hz, 1H), 6.95 (t, J=7.0 Hz, 1H), 6.73 (s, 1H), 5.04 (s, 2H), 4.78 (s, 2H), 4.53 (s, 2H). ES-MS [M+H]⁺=407.

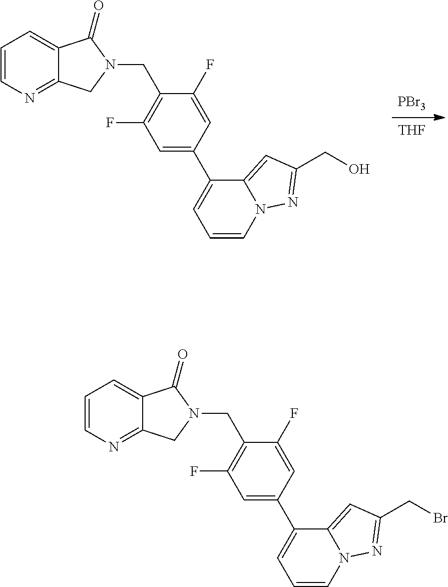 [SMILES: O=C1c2cccnc2CN1Cc1c(F)cc(-c2cccn3nc(CO)cc23)cc1F ; "PBr3" ; O=C1c2cccnc2CN1Cc1c(F)cc(-c2cccn3nc(CBr)cc23)cc1F ; "THF"]

6-(4-(2-(Bromomethyl)pyrazolo[1,5-a]pyridin-4-yl)-2,6-difluorobenzyl)-6,7-dihydro-5H-pyrrolo[3,4-b]pyridin-5-one. To a solution of 6-(2,6-difluoro-4-(2-(hydroxymethyl)pyrazolo[1,5-a]pyridin-4-yl)benzyl)-6,7-dihydro-5H-pyrrolo[3,4-b]pyridin-5-one (103 mg, 0.25 mmol) in THE (6 mL), PBr₃ (0.71 mL, 7.57 mmol) was added and stirred at rt. After 9 h, the reaction mixture was poured on ice/DCM mixture and extracted with DCM (3×50 mL). The aqueous layer was then neutralized with K₂CO₃ and extracted again with DCM (3×50 mL). The combined organic layers were passed through a phase separator and concentrated in vacuo. The crude residue was used for the next step without further purification. ES-MS [M+H]⁺=469 and 471.

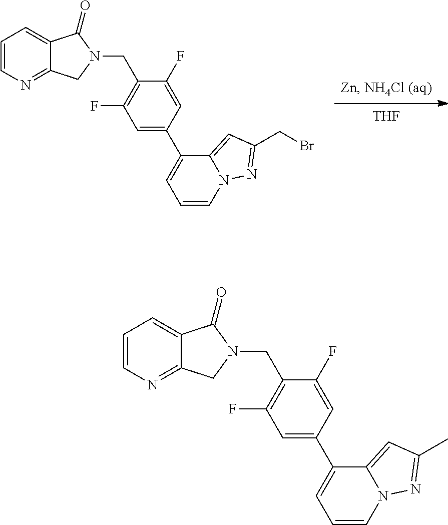 [SMILES: O=C1c2cccnc2CN1Cc1c(F)cc(-c2cccn3nc(CBr)cc23)cc1F ; "Zn, NH4Cl (aq)" ; Cc1cc2c(-c3cc(F)c(CN4Cc5ncccc5C4=O)c(F)c3)cccn2n1 ; "THF"]

6-(2,6-Difluoro-4-(2-methylpyrazolo[1,5-a]pyridin-4-yl)benzyl)-6,7-dihydro-5H-pyrrolo[3,4-b]pyridin-5-one. To a solution of 6-(4-(2-(bromomethyl)pyrazolo[1,5-a]pyridin-4-yl)-2,6-difluorobenzyl)-6,7-dihydro-5H-pyrrolo[3,4-b]pyridin-5-one (100 mg, 0.21 mmol) in THF (3 mL) was added activated Zn (21 mg, 0.32 mmol) at rt followed by saturated aqueous NH₄Cl solution (3 mL). After 1 h at rt, the reaction mixture was diluted with H₂O (1 mL) and extracted with DCM (3×10 mL). The combined organics were passed through a phase separator and concentrated in vacuo. The crude residue was then purified by reverse phase HPLC to give the title compound (32 mg). ¹H-NMR (400 MHz, MeOD) δ 8.73 (dd, J=5.0, 1.6 Hz, 1H), 8.44 (d, J=7.0 Hz, 1H), 8.18 (dd, J=7.8, 1.6 Hz, 1H), 7.55 (dd, J=7.8, 5.0 Hz, 1H), 7.45-7.33 (m, 2H), 7.30 (dd, J=7.1, 1.0 Hz, 1H), 6.90 (t, J=7.0 Hz, 1H), 6.54 (s, 1H), 5.03 (s, 2H), 4.53 (s, 2H), 2.45 (s, 3H). ES-MS [M+H]⁺=391.

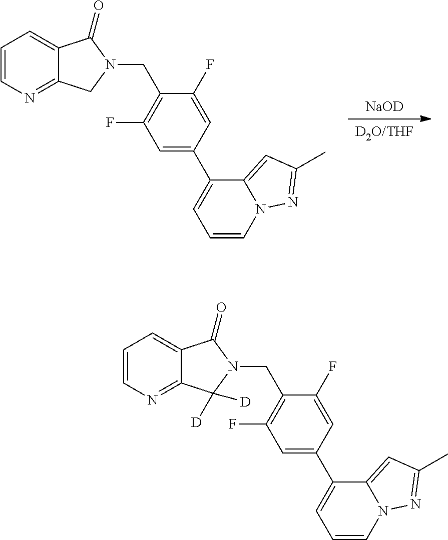 [SMILES: Cc1cc2c(-c3cc(F)c(CN4Cc5ncccc5C4=O)c(F)c3)cccn2n1 ; "NaOD" ; "D2O/THF" ; [2H]C1([2H])c2ncccc2C(=O)N1Cc1c(F)cc(-c2cccn3nc(C)cc23)cc1F]

6-(2,6-Difluoro-4-(2-methylpyrazolo[1,5-a]pyridin-4-yl)benzyl)-6,7-dihydro-5H-pyrrolo[3,4-b]pyridin-5-one-7,7-d₂ (Compound 235). 6-(2,6-Difluoro-4-(2-methylpyrazolo[1,5-a]pyridin-4-yl)benzyl)-6,7-dihydro-5H-pyrrolo[3,4-b]pyridin-5-one (32 mg, 0.08 mmol) was dissolved in THF (1 mL). To this reaction mixture, D₂O (1.06 mL) and sodium deuteroxide (3 μL, 0.04 mmol) were added. The resulting reaction mixture was stirred at 35° C. After 4 h, the reaction mixture was extracted with DCM (3×10 mL). The combined organic extracts were dried over Na₂SO₄, filtered, and concentrated to give the title compound (28 mg, 86%, >98% D by ¹H-NMR). ¹H-NMR (400 MHz, CDCl₃) δ 8.70 (dd, J=4.9, 1.7 Hz, 1H), 8.37 (d, J=6.9 Hz, 1H), 8.12 (dd, J=7.7, 1.6 Hz, 1H), 7.38 (dd, J=7.7, 4.9 Hz, 1H), 7.27-7.21 (m, 2H), 7.07 (dd, J=7.0, 1.1 Hz, 1H), 6.74 (t, J=7.0 Hz, 1H), 6.42 (s, 1H), 5.00 (s, 2H), 2.48 (s, 3H). ES-MS [M+H]⁺=393.

### Example 10. 6-(2,6-Difluoro-4-(2-(methyl-d₃)pyrazolo[1,5-a]pyridin-4-yl)benzyl)-6,7-dihydro-5H-pyrrolo[3,4-b]pyridin-5-one-7,7-d₂ (Compound 236)

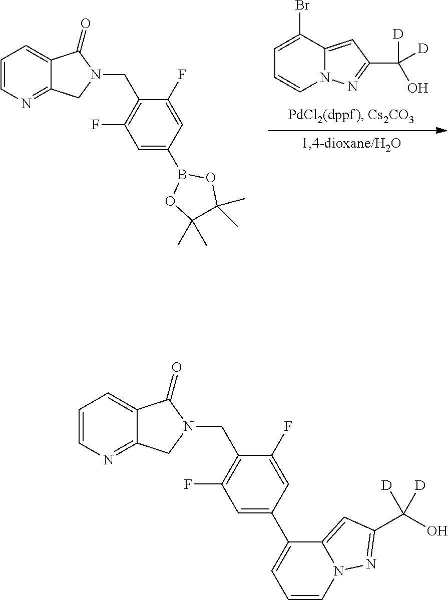

6-(2,6-Difluoro-4-(2-(hydroxymethyl-d₂)pyrazolo[1,5-a]pyridin-4-yl)benzyl)-6,7-dihydro-5H-pyrrolo[3,4-b]pyridin-5-one. The title compound was prepared in an analogous manner as in Example 9, using 6-(2,6-difluoro-4-(4,4,5,5-tetramethyl-1,3,2-dioxaborolan-2-yl)benzyl)-6,7-dihydro-5H-pyrrolo[3,4-b]pyridin-5-one (76 mg, 0.20 mmol) and (4-bromopyrazolo[1,5-a]pyridin-2-yl)methan-d₂-ol (34.7 mg, 0.15 mmol). The title compound was obtained (50 mg). ¹H NMR (400 MHz, MeOD) δ 8.73 (d, J=3.4 Hz, 1H), 8.50 (d, J=7.0 Hz, 1H), 8.18 (d, J=7.8 Hz, 1H), 7.55 (dd, J=7.8, 5.0 Hz, 1H), 7.42 (d, J=8.4 Hz, 2H), 7.33 (d, J=6.5 Hz, 1H), 6.95 (t, J=7.0 Hz, 1H), 6.73 (s, 1H), 5.04 (s, 2H), 4.53 (s, 2H). ES-MS [M+H]⁺=409.

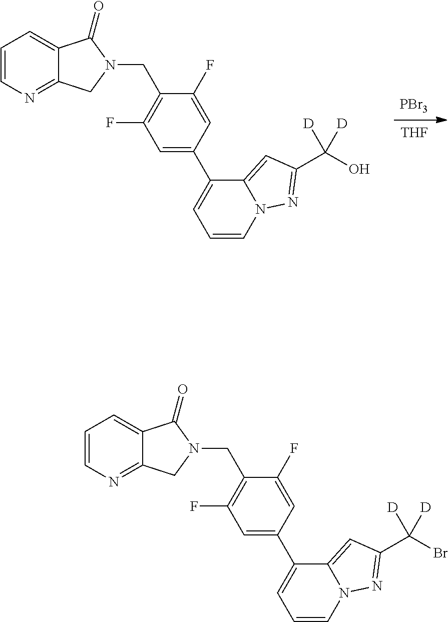 [SMILES: [2H]C([2H])(O)c1cc2c(-c3cc(F)c(CN4Cc5ncccc5C4=O)c(F)c3)cccn2n1 ; "PBr3" ; [2H]C([2H])(Br)c1cc2c(-c3cc(F)c(CN4Cc5ncccc5C4=O)c(F)c3)cccn2n1 ; "THF"]

6-(4-(2-(Bromomethyl-d₂)pyrazolo[1,5-a]pyridin-4-yl)-2,6-difluorobenzyl)-6,7-dihydro-5H-pyrrolo[3,4-b]pyridin-5-one. The title compound was prepared in an analogous manner as in Example 9, using 6-(2,6-difluoro-4-(2-(hydroxymethyl-d₂)pyrazolo[1,5-a]pyridin-4-yl)benzyl)-6,7-dihydro-5H-pyrrolo[3,4-b]pyridin-5-one (50 mg, 0.12 mmol) and PBr₃ (0.69 mL, 7.36 mmol). The crude residue was used for the next step without further purification. ES-MS [M+H]f=471 and 473.

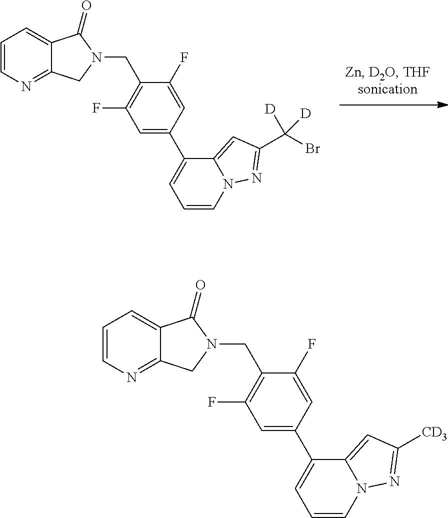 [SMILES: [2H]C([2H])(Br)c1cc2c(-c3cc(F)c(CN4Cc5ncccc5C4=O)c(F)c3)cccn2n1 ; [2H]C([2H])([2H])c1cc2c(-c3cc(F)c(CN4Cc5ncccc5C4=O)c(F)c3)cccn2n1]

6-(2,6-Difluoro-4-(2-(methyl-d₃)pyrazolo[1,5-a]pyridin-4-yl)benzyl)-6,7-dihydro-5H-pyrrolo[3,4-b]pyridin-5-one. To a solution of 6-(4-(2-(bromomethyl-d₂)pyrazolo[1,5-a]pyridin-4-yl)-2,6-difluorobenzyl)-6,7-dihydro-5H-pyrrolo[3,4-b]pyridin-5-one (50 mg, 0.11 mmol) in THE (0.5 mL), D₂O (0.5 mL) and Zn (28 mg, 0.42 mmol) were added and sonicated. After 5 min, the reaction mixture was diluted with H₂O (0.5 mL) and extracted with DCM (3×10 mL). The combined organics were passed through a phase separator and concentrated in vacuo. The crude residue was then purified by reverse phase HPLC to give the title compound (4.9 mg, 11%, >98% D by ¹H-NMR). ¹H-NMR (400 MHz, MeOD) δ 8.72 (dd, J=5.0, 1.5 Hz, 1H), 8.44 (d, J=7.0 Hz, 1H), 8.18 (dd, J=7.8, 1.6 Hz, 1H), 7.55 (dd, J=7.8, 5.0 Hz, 1H), 7.45-7.33 (m, 2H), 7.29 (dd, J=7.1, 1.0 Hz, 1H), 6.90 (t, J=7.0 Hz, 1H), 6.54 (d, J=1.0 Hz, 1H), 5.03 (s, 2H), 4.52 (s, 2H). ES-MS [M+H]⁺=394.

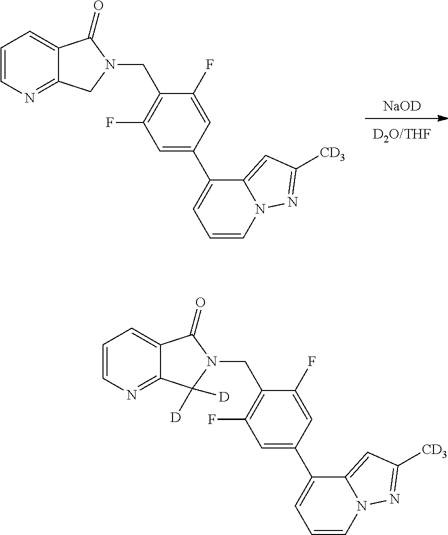 [SMILES: [2H]C([2H])([2H])c1cc2c(-c3cc(F)c(CN4Cc5ncccc5C4=O)c(F)c3)cccn2n1 ; "NaOD" ; [2H]C([2H])([2H])c1cc2c(-c3cc(F)c(CN4C(=O)c5cccnc5C4([2H])[2H])c(F)c3)cccn2n1]

6-(2,6-Difluoro-4-(2-(methyl-d₃)pyrazolo[1,5-a]pyridin-4-yl)benzyl)-6,7-dihydro-5H-pyrrolo[3,4-b]pyridin-5-one-7,7-d₂ (Compound 236). The title compound was prepared in an analogous manner as in Example 9, using 6-(2,6-difluoro-4-(2-(methyl-d₃)pyrazolo[1,5-a]pyridin-4-yl)benzyl)-6,7-dihydro-5H-pyrrolo[3,4-b]pyridin-5-one (6.2 mg, 0.02 mmol). The title compound was obtained (4.6 mg, >98% D by ¹H-NMR). ¹H NMR (400 MHz, CDCl₃) δ 8.72 (dd, J=4.9, 1.6 Hz, 1H), 8.40 (d, J=7.0 Hz, 1H), 8.14 (dd, J=7.7, 1.6 Hz, 1H), 7.40 (dd, J=7.7, 5.0 Hz, 1H), 7.30-7.21 (m, 2H), 7.09 (dd, J=7.1, 1.0 Hz, 1H), 6.76 (t, J=7.0 Hz, 1H), 6.44 (d, J=1.0 Hz, 1H), 5.01 (s, 2H). ES-MS [M+H]⁺=396.

### Example 11. 6-(2-Fluoro-6-methyl-4-(2-methyl-2H-indazol-4-yl)benzyl)-6,7-dihydro-5H-pyrrolo[3,4-b]pyridin-5-one-7,7-d₂ (Compound 155)

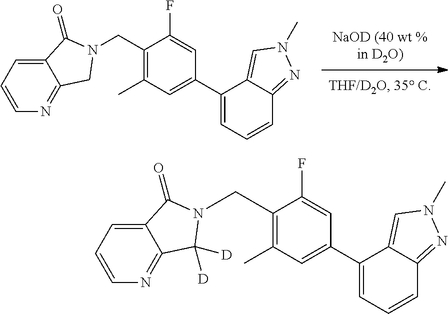 [SMILES: Cc1cc(-c2cccc3nn(C)cc23)cc(F)c1CN1Cc2ncccc2C1=O ; "NaOD (40 wt %in D2O)" ; [2H]C1([2H])c2ncccc2C(=O)N1Cc1c(C)cc(-c2cccc3nn(C)cc23)cc1F]

To a suspension of 6-(2-fluoro-6-methyl-4-(2-methyl-2H-indazol-4-yl)benzyl)-6,7-dihydro-5H-pyrrolo[3,4-b]pyridin-5-one (1.56 g, 4.04 mmol, 1.0 eq.) in THE (27 mL) was added D₂O (14.1 mL) followed by a solution of sodium deuteroxide (40 wt % in D₂O, 0.13 mL, 2.02 mmol, 5.0 eq.). The reaction was stirred at 35° C. After 48 h, the reaction mixture was extracted with DCM (3×100 mL). The combined organic layers were dried (Na₂SO₄), filtered and concentrated. Purification using normal phase chromatography (0-10% MeOH/DCM) provided the title compound (1.08 g, 69% yield, >98% deuterium incorporation by HRMS and ¹H NMR). H NMR (400 MHz, DMSO-d₆) δ 8.74 (dd, J=4.9, 1.6 Hz, 1H), 8.57 (d, J=1.0 Hz, 1H), 8.13 (dd, J=7.7, 1.6 Hz, 1H), 7.61 (dt, J=8.6, 0.9 Hz, 1H), 7.53 (dd, J=7.7, 4.9 Hz, 1H), 7.47-7.38 (m, 2H), 7.31 (dd, J=8.6, 6.9 Hz, 1H), 7.21 (dd, J=7.0, 0.8 Hz, 1H), 4.89 (d, J=1.8 Hz, 2H), 4.18 (s, 3H), 2.50 (s, 3H); ES-MS [M+H]⁺=389.1.

### Example 12. 6-(2-Fluoro-6-methyl-4-(2-(methyl-d₃)-2H-indazol-4-yl)benzyl)-6,7-dihydro-5H-pyrrolo[3,4-b]pyridin-5-one-7,7-d₂ (Compound 156)

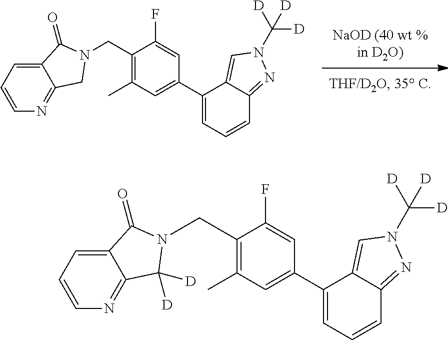 [SMILES: [2H]C([2H])([2H])n1cc2c(-c3cc(C)c(CN4Cc5ncccc5C4=O)c(F)c3)cccc2n1 ; "NaOD (40 wt %in D2O)" ; [2H]C1([2H])c2ncccc2C(=O)N1Cc1c(C)cc(-c2cccc3nn(C([2H])([2H])[2H])cc23)cc1F]

To a suspension of 6-(2-fluoro-6-methyl-4-(2-(methyl-d₃)-2H-indazol-4-yl)benzyl)-6,7-dihydro-5H-pyrrolo[3,4-b]pyridin-5-one (0.33 g, 0.85 mmol, 1.0 eq.) in THE (8.5 mL) was added D₂O (2.1 mL) followed by a solution of sodium deuteroxide (40 wt % in D₂O, 57 μL, 0.85 mmol, 1.0 eq.). The reaction was stirred at 35° C. After 24 h, the reaction mixture was extracted with EtOAc (3×20 mL). The combined organic layers were dried (Na₂SO₄), filtered and concentrated. Purification using normal phase chromatography (0-10% MeOH/DCM) provided the title compound (0.30 g, 89% yield, >94% deuterium incorporation by HRMS and ¹H NMR). ¹H NMR (400 MHz, DMSO-d₆) δ 8.74 (dd, J=4.9, 1.6 Hz, 1H), 8.57 (d, J=1.0 Hz, 1H), 8.13 (dd, J=7.7, 1.6 Hz, 1H), 7.61 (dt, J=8.6, 0.9 Hz, 1H), 7.53 (dd, J=7.7, 4.9 Hz, 1H), 7.45 (d, J=1.8 Hz, 1H), 7.42 (dd, J=10.9, 1.8 Hz, 1H), 7.32 (dd, J=8.6, 6.9 Hz, 1H), 7.21 (dd, J=7.0, 0.8 Hz, 1H), 4.89 (d, J=1.7 Hz, 2H), 2.50 (s, 3H); ES-MS [M+H]⁺=392.1.

### Example 13. 6-(2-Cyclopropyl-6-fluoro-4-(2-(methyl-d₃)-2H-indazol-4-yl)benzyl)-6,7-dihydro-5H-pyrrolo[3,4-b]pyridin-5-one-7,7-d₂ (Compound 190)

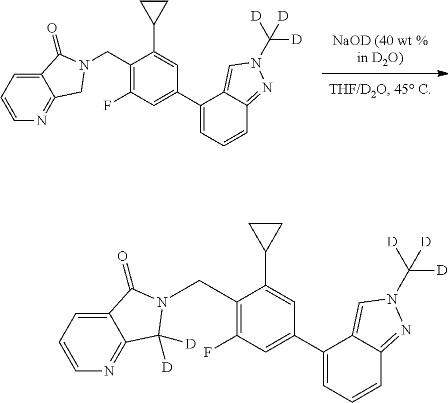 [SMILES: [2H]C([2H])([2H])n1cc2c(-c3cc(F)c(CN4Cc5ncccc5C4=O)c(C4CC4)c3)cccc2n1 ; "NaOD (40 wt %in D2O)" ; [2H]C1([2H])c2ncccc2C(=O)N1Cc1c(F)cc(-c2cccc3nn(C([2H])([2H])[2H])cc23)cc1C1CC1]

To a suspension of 6-(2-cyclopropyl-6-fluoro-4-(2-(methyl-d₃)-2H-indazol-4-yl)benzyl)-6,7-dihydro-5H-pyrrolo[3,4-b]pyridin-5-one (0.22 g, 0.53 mmol, 1.0 eq.) in THE (3 mL) was added D₂O (5 mL) followed by a solution of sodium deuteroxide (40 wt % in D₂O, 72 μL, 1.06 mmol, 2.0 eq.). The reaction was stirred at 45° C. After 24 h, the reaction mixture was extracted with EtOAc (3×20 mL). The combined organic layers were dried (Na₂SO₄), filtered and concentrated. Purification using normal phase chromatography (0-5% MeOH/DCM) provided the title compound (0.21 g, 93% yield, >95% deuterium incorporation by ¹H NMR). ¹H NMR (400 MHz, CDCl₃) δ 8.71 (dd, J=5.0, 1.5 Hz, 1H), 8.13 (dd, J=7.7, 1.4 Hz, 1H), 7.98 (s, 1H), 7.71 (d, J=8.6 Hz, 1H), 7.44-7.30 (m, 2H), 7.27-7.24 (m, 1H), 7.15-7.09 (m, 1H), 7.07-7.02 (i, 1H), 5.18 (d, J=1.9 Hz, 2H), 2.32-2.21 (m, 1H), 1.10-0.99 (m, 2H), 0.75-0.66 (n, 2H); ES-MS [M+H]⁺=418.2.

The compounds presented in Table 19 may be prepared in an analogous manner to those described in the preceding schemes and examples using appropriate starting materials.

TABLE 19

| No. | STRUCTURE | NAME | ¹H -NMR and/or ES-MS [M + H]⁺ |
|---|---|---|---|
| 1 | 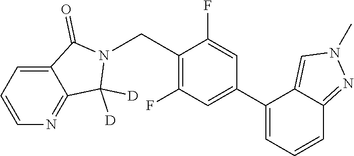 [SMILES: [2H]C1([2H])c2ncccc2C(=O)N1Cc1c(F)cc(-c2cccc3nn(C)cc23)cc1F] | 6-(2,6-difluoro-4-(2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 8.04 (s, 1H), 7.74 (d, J = 8.7 Hz, 1H), 7.44-7.31 (m, 2H), 7.30-7.21 (m, 2H), 7.15 (dd, J = 6.9, 0.7 Hz, 1H), 5.02 (s, 2H)., 42.5 (s, 3H); ES-MS [M + H]⁺ = 393.3. |
| 2 | 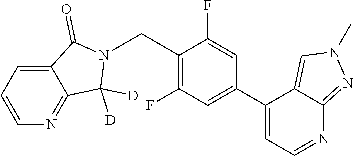 [SMILES: [2H]C1([2H])c2ncccc2C(=O)N1Cc1c(F)cc(-c2ccnc3nn(C)cc23)cc1F] | 6-(2,6-difluoro-4-(2- methyl-2H-pyrazolo[3,4- b]pyridin-4-yl)benzyl)- 6,7-dihydro-5H- pyrrolo[3,4-b]pyridin-5- one-7,7-d₂ | ¹H NMR (400 MHz, CDCl₃) δ 8.76 (d, J = 4.5 Hz, 1H), 8.72 (dd, J = 4.9, 1.6 Hz, 1H), 8.15 (dd, J = 7.7, 1.6 Hz, 1H), 8.07 (s, 1H), 7.41 (dd, J = 7.7, 4.9 Hz, 1H), 7.36-7.27 (m, 2H), 7.12 (d, J = 4.5 Hz, 1H), 5.03 (s, 2H), 4.30 (s, 3H); ES-MS [M + H]⁺ = 394.4. |
| 3 | 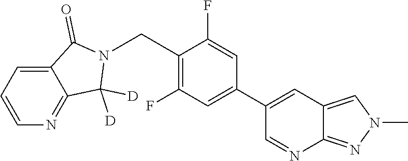 [SMILES: [2H]C1([2H])c2ncccc2C(=O)N1Cc1c(F)cc(-c2cnc3nn(C)cc3c2)cc1F] | 6-(2,6-difluoro-4-(2- methyl-2H-pyrazolo[3,4- b]pyridin-5-yl)benzyl)- 6,7-dihydro-5H- pyrrolo[3,4-b]pyridin-5- one-7,7-d₂ | ¹H NMR (400 MHz, CDCl₃) δ 8.89 (d, J = 2.4 Hz, 1H), 8.71 (dd, J = 5.0, 1.6 Hz, 1H), 8.18-8.10 (m, 2H), 7.99 (s, 1H), 7.40 (dd, J = 7.7, 4.9 Hz, 1H), 7.24-7.19 (m, 2H), 5.00 (s, 2H), 4.30 (s, 3H); ES-MS [M + H]⁺ = 394.4. |
| 4 | 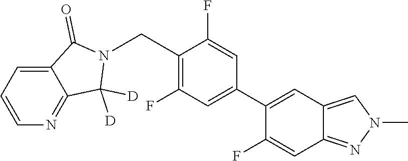 [SMILES: [2H]C1([2H])c2ncccc2C(=O)N1Cc1c(F)cc(-c2cc3cn(C)nc3cc2F)cc1F] | 6-(2,6-difluoro-4-(6- fluoro-2-methyl-2H- indazol-5-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 7.96 (s, 1H), 7.67 (d, J = 7.5 Hz, 1H), 7.44-7.37 (m, 2H), 7.21-7.13 (m, 2H), 5.00 (s, 2H), 4.23 (s, 3H); ES-MS [M + H]⁺ = 411.3. |
| 5 | 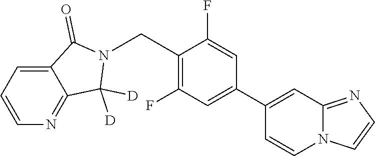 [SMILES: [2H]C1([2H])c2ncccc2C(=O)N1Cc1c(F)cc(-c2ccn3ccnc3c2)cc1F] | 6-(2,6-difluoro-4- (imidazo[1,2-a]pyridin- 7-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.21 (dd, J = 7.1, 0.9 Hz, 1H), 8.14 (dd, J = 7.7, 1.6 Hz, 1H), 7.81 (dt, J = 1.8, 0.9 Hz, 1H), 7.71 (d, J = 1.2 Hz, 1H), 7.63 (s, 1H), 7.40 (dd, J = 7.7, 4.9 Hz, 1H), 7.26-7.21 (m, 2H), 7.00 (dd, J = 7.1, 1.9 Hz, 1H), 5.00 (s, 2H); ES-MS [M + H]⁺ = 407.3. |
| 6 | 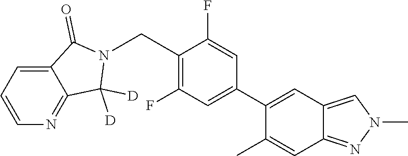 [SMILES: [2H]C1([2H])c2ncccc2C(=O)N1Cc1c(F)cc(-c2cc3cn(C)nc3cc2C)cc1F] | 6-(4-(2,6-dimethyl-2H- indazol-5-yl)-2,6- difluorobenzyl)-6,7- dihydro-5H-pyrrolo[3,4- b]pyridin-5-one-7,7-d₂ | ¹H NMR (400 MHz, CDCl₃) δ 8.71 (dd, J = 4.9, 1.6 Hz, 1H), 8.14 (dd, J = 7.7, 1.6 Hz, 1H), 7.87 (s, 1H), 7.54 (s, 1H), 7.44 (s, 1H), 7.40 (dd, J = 7.7, 4.9 Hz, 1H), 6.99-6.90 (m, 2H), 5.01 (s, 2H), 4.21 (s, 3H), 2.31 (s, 3H); ES-MS [M + H]⁺ = 407.3. |
| 7 | 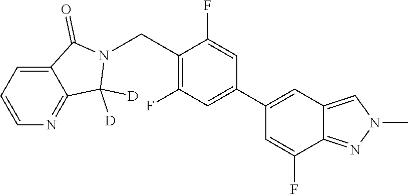 [SMILES: [2H]C1([2H])c2ncccc2C(=O)N1Cc1c(F)cc(-c2cc(F)c3nn(C)cc3c2)cc1F] | 6-(2,6-difluoro-4-(7- fluoro-2-methyl-2H- indazol-5-yl)benzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.5 Hz, 1H), 8.14 (dd, J = 7.7, 1.7 Hz, 1H), 8.02 (d, J = 2.6 Hz, 1H), 7.60 (s, 1H), 7.39 (dd, J = 7.7, 4.9 Hz, 1H), 7.26-7.10 (m, 3H), 4.99 (s, 2H), 4.27 (s, 3H); ES-MS [M + H]⁺ = 411.3. |
| 8 | 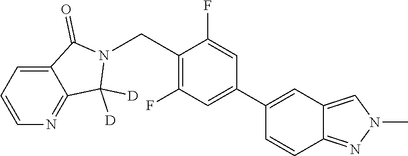 [SMILES: [2H]C1([2H])c2ncccc2C(=O)N1Cc1c(F)cc(-c2ccc3nn(C)cc3c2)cc1F] | 6-(2,6-difluoro-4-(2- methyl-2H-indazol-5- yl)benzyl)-6,7-dihydro- 5H-pyrrolo[3,4- b]pyridin-5-one-7,7-d₂ | ¹H NMR (400 MHz, CDCl₃) δ 8.70 (dd, J = 5.0, 1.6 Hz, 1H), 8.14 (dd, J = 7.7, 1.6 Hz, 1H), 7.96 (d, J = 13.3 Hz, 1H), 7.87-7.73 (m, 3H), 7.47 (dd, J = 9.0, 1.8 Hz, 1H), 7.39 (dd, J = 7.7, 5.0 Hz, 1H), 7.22 (d, J = 8.5 Hz, 1H), 4.99 (s, 2H), 4.25 (d, J = 1.4 Hz, 3H); ES-MS [M + H]⁺ = 393.4. |
| 9 | 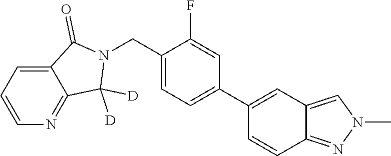 [SMILES: [2H]C1([2H])c2ncccc2C(=O)N1Cc1ccc(-c2ccc3nn(C)cc3c2)cc1F] | 6-(2-fluoro-4-(2-methyl- 2H-indazol-5-yl)benzyl)- 6,7-dihydro-5H- pyrrolo[3,4-b]pyridin-5- one-7,7-d₂ | 1H NMR (400 MHz, CDCl₃) δ 8.72 (dd, J = 4.9, 1.6 Hz, 1H), 8.15 (dd, J = 7.7, 1.6 Hz, 1H), 7.91 (s, 1H), 7.85 (dd, J = 1.6, 0.8 Hz, 1H), 7.71 (dd, J = 8.7, 0.9 Hz, 1H), 7.50- 7.36 (m, 4H), 7.30 (dd, J = 8.7, 1.5 Hz, 1H), 4.94 (d, J = 1.2 Hz, 2H), 4.24 (s, 3H); ES- MS [M + H]⁺ = 375.4. |
| 10 | 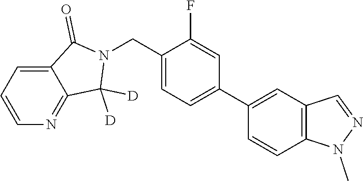 [SMILES: [2H]C1([2H])c2ncccc2C(=O)N1Cc1ccc(-c2ccc3c(cnn3C)c2)cc1F] | 6-(2-fluoro-4-(1-methyl- 1H-indazol-5-yl)benzyl)- 6,7-dihydro-5H- pyrrolo[3,4-b]pyridin-5- one-7,7-d₂ | ES-MS [M + H]⁺ = 375.4 |
| 11 | 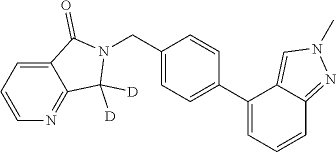 [SMILES: [2H]C1([2H])c2ncccc2C(=O)N1Cc1ccc(-c2cccc3nn(C)cc23)cc1] | 6-(4-(2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.18 (dd, J = 7.7, 1.6 Hz, 1H), 8.01 (s, 1H), 7.68-7.61 (m, 3H), 7.49- 7.39 (m, 3H), 7.35 (dd, J = 8.7, 6.9 Hz, 1H), 7.13 (dd, J = 6.9, 0.8 Hz, 1H), 4.90 (s, 2H), 4.23 (s, 3H); ES-MS [M + H]⁺ = 357.4. |
| 12 | 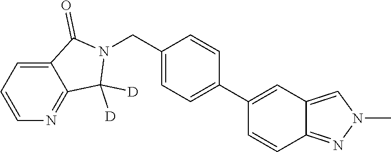 [SMILES: [2H]C1([2H])c2ncccc2C(=O)N1Cc1ccc(-c2ccc3nn(C)cc3c2)cc1] | 6-(4-(2-methyl-2H- indazol-5-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 7.94 (s, 1H), 7.80 (dd, J = 1.7, 0.9 Hz, 1H), 7.75 (d, J = 9.0 Hz, 1H), 7.65-7.58 (m, 2H), 7.53 (dd, J = 9.0, 1.7 Hz, 1H), 7.45- 7.37 (m, 3H), 4.88 (s, 2H), 4.24 (s, 3H); ES-MS [M + H]⁺ = 357.4. |
| 13 | 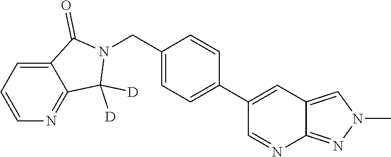 [SMILES: [2H]C1([2H])c2ncccc2C(=O)N1Cc1ccc(-c2cnc3nn(C)cc3c2)cc1] | 6-(4-(2-methyl-2H- pyrazolo[3,4-b]pyridin- 5-yl)benzyl)-6,7- dihydro-5H-pyrrolo[3,4- b]pyridin-5-one-7,7-d₂ | ¹H NMR (400 MHz, CDCl₃) δ 8.93 (d, J = 2.3 Hz, 1H), 8.72 (dd, J = 5.0, 1.6 Hz, 1H), 8.17 (dd, J = 7.7, 1.6 Hz, 1H), 8.13 (d, J = 2.3 Hz, 1H), 7.95 (s, 1H), 7.64-7.57 (m, 2H), 7.48-7.38 (m, 3H), 4.90 (s, 2H), 4.28 (s, 3H); ES-MS [M + H]⁺ = 358.3. |
| 14 | 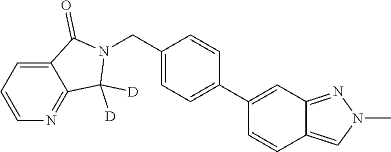 [SMILES: [2H]C1([2H])c2ncccc2C(=O)N1Cc1ccc(-c2ccc3cn(C)nc3c2)cc1] | 6-(4-(2-methyl-2H- indazol-6-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 7.90 (d, J = 0.9 Hz, 1H), 7.85 (dt, J = 1.7, 1.0 Hz, 1H), 7.70 (dd, J = 8.7, 0.9 Hz, 1H), 7.69-7.61 (m, 2H), 7.45-7.37 (m, 3H), 7.33 (dd, J = 8.7, 1.5 Hz, 1H), 4.89 (s, 2H), 4.24 (s, 3H); ES-MS [M + H]⁺ = 357.4. |
| 15 | 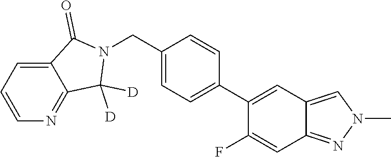 [SMILES: [2H]C1([2H])c2ncccc2C(=O)N1Cc1ccc(-c2cc3cn(C)nc3cc2F)cc1] | 6-(4-(6-fluoro-2-methyl- 2H-indazol-5-yl)benzyl)- 6,7-dihydro-5H- pyrrolo[3,4-b]pyridin-5- one-7,7-d₂ | ¹H NMR (400 MHz, CDCl₃) δ 8.72 (dd, J = 4.9, 1.6 Hz, 1H), 8.17 (dd, J = 7.7, 1.6 Hz, 1H), 7.92 (s, 1H), 7.64 (d, J = 7.5 Hz, 1H), 7.54 (dd, J = 8.3, 1.9 Hz, 2H), 7.45-7.34 (m, 4H), 4.89 (s, 2H), 4.21 (s, 3H); ES-MS [M + H]⁺ = 375.4. |
| 16 | 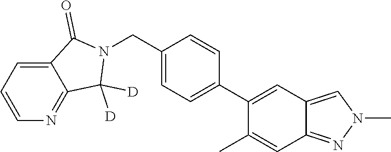 [SMILES: [2H]C1([2H])c2ncccc2C(=O)N1Cc1ccc(-c2cc3cn(C)nc3cc2C)cc1] | 6-(4-(2,6-dimethyl-2H- indazol-5-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.17 (dd, J = 7.7, 1.6 Hz, 1H), 7.84 (s, 1H), 7.54 (d, J = 1.0 Hz, 1H), 7.46-7.29 (m, 6H), 4.89 (s, 2H), 4.21 (s, 3H), 2.28 (s, 3H); ES-MS [M + H]⁺ = 371.4. |
| 17 | 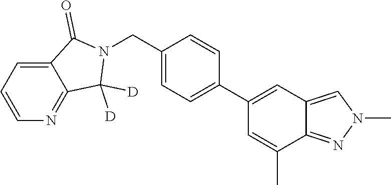 [SMILES: [2H]C1([2H])c2ncccc2C(=O)N1Cc1ccc(-c2cc(C)c3nn(C)cc3c2)cc1] | 6-(4-(2,7-dimethyl-2H- indazol-5-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 7.92 (s, 1H), 7.66-7.57 (m, 3H), 7.46- 7.35 (m, 3H), 7.29 (dq, J = 2.1, 1.1 Hz, 1H), 4.88 (s, 2H), 4.25 (s, 3H), 2.67 (s, 3H); ES-MS [M + H]⁺ = 371.3. |
| 18 | 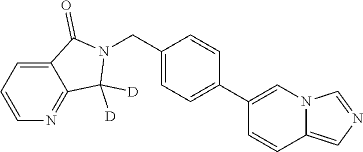 [SMILES: [2H]C1([2H])c2ncccc2C(=O)N1Cc1ccc(-c2ccc3cncn3c2)cc1] | 6-(4-(imidazo[1,5- a]pyridin-6-yl)benzyl)- 6,7-dihydro-5H- pyrrolo[3,4-b]pyridin-5- one-7,7-d₂ | ¹H NMR (400 MHz, CDCl₃) δ 8.72 (dd, J = 5.0, 1.6 Hz, 1H), 8.21-8.15 (m, 1H), 8.16 (s, 1H), 8.08 (d, J = 1.3 Hz, 1H), 7.58- 7.48 (m, 3H), 7.44-7.42 (m, 4H), 6.96 (dd, J = 9.5, 1.5 Hz, 1H), 4.89 (s, 2H); ES-MS [M + H]⁺ = 343.4. |
| 19 | 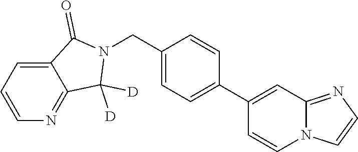 [SMILES: [2H]C1([2H])c2ncccc2C(=O)N1Cc1ccc(-c2ccn3ccnc3c2)cc1] | 6-(4-(imidazo[1,2- a]pyridin-7-yl)benzyl)- 6,7-dihydro-5H- pyrrolo[3,4-b]pyridin-5- one-7,7-d₂ | ¹H NMR (400 MHz, CDCl₃) δ 8.72 (dd, J = 5.0, 1.6 Hz, 1H), 8.21-8.14 (m, 2H), 7.81 (s, 1H), 7.69-7.61 (m, 3H), 7.59 (s, 1H), 7.47-7.38 (m, 3H), 7.06 (dd, J = 7.1, 1.8 Hz, 1H), 4.89 (s, 2H); ES-MS [M + H]⁺ = 343.4. |
| 20 | 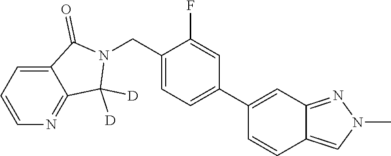 [SMILES: [2H]C1([2H])c2ncccc2C(=O)N1Cc1ccc(-c2ccc3cn(C)nc3c2)cc1F] | 6-(2-fluoro-4-(2-methyl- 2H-indazol-6-yl)benzyl)- 6,7-dihydro-5H- pyrrolo[3,4-b]pyridin-5- one-7,7-d₂ | ES-MS [M + H]⁺ = 375.4 |
| 21 | 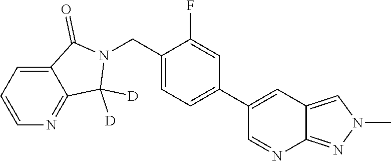 [SMILES: [2H]C1([2H])c2ncccc2C(=O)N1Cc1ccc(-c2cnc3nn(C)cc3c2)cc1F] | 6-(2-fluoro-4-(2-methyl- 2H-pyrazolo[3,4- b]pyridin-5-yl)benzyl)- 6,7-dihydro-5H- pyrrolo[3,4-b]pyridin-5- one-7,7-d₂ | ¹H NMR (400 MHz, CDCl3) δ 8.91 (d, J = 2.3 Hz, 1H), 8.72 (dd, J = 5.0, 1.6 Hz, 1H), 8.19-8.11 (m, 2H), 7.97 (s, 1H), 7.49 (t, J = 7.8 Hz, 1H), 7.44-7.33 (m, 3H), 4.98-4.93 (m, 2H), 4.29 (s, 3H); ES-MS [M + H]⁺ = 376.4. |
| 22 | 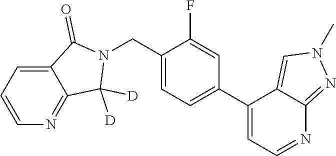 [SMILES: [2H]C1([2H])c2ncccc2C(=O)N1Cc1ccc(-c2ccnc3nn(C)cc23)cc1F] | 6-(2-fluoro-4-(2-methyl- 2H-pyrazolo[3,4- b]pyridin-4-yl)benzyl)- 6,7-dihydro-5H- pyrrolo[3,4-b]pyridin-5- one-7,7-d₂ | ES-MS [M + H]⁺ = 376.2. |
| 23 | 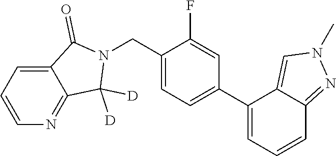 [SMILES: [2H]C1([2H])c2ncccc2C(=O)N1Cc1ccc(-c2cccc3nn(C)cc23)cc1F] | 6-(2-fluoro-4-(2-methyl- 2H-indazol-4-yl)benzyl)- 6,7-dihydro-5H- pyrrolo[3,4-b]pyridin-5- one-7,7-d₂ | ES-MS [M + H]⁺ = 375.3. |
| 24 | 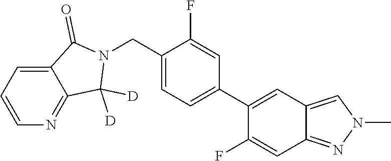 [SMILES: [2H]C1([2H])c2ncccc2C(=O)N1Cc1ccc(-c2cc3cn(C)nc3cc2F)cc1F] | 6-(2-fluoro-4-(6-fluoro- 2-methyl-2H-indazol-5- yl)benzyl)-6,7-dihydro- 5H-pyrrolo[3,4- b]pyridin-5-one-7,7-d₂ | ¹H NMR (400 MHz, CDCl₃) δ 8.72 (dd, J = 5.0, 1.6 Hz, 1H), 8.14 (dd, J = 7.7, 1.6 Hz, 1H), 7.94 (s, 1H), 7.64 (d, J = 7.5 Hz, 1H), 7.48-7.35 (m, 3H), 7.31 (dd, J = 9.5, 1.8 Hz, 2H), 4.94 (d, J = 1.2 Hz, 2H), 4.21 (s, 3H); ES-MS [M + H]⁺ = 393.3. |
| 25 | 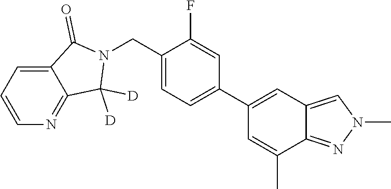 [SMILES: [2H]C1([2H])c2ncccc2C(=O)N1Cc1ccc(-c2cc(C)c3nn(C)cc3c2)cc1F] | 6-(4-(2,7-dimethyl-2H- indazol-5-yl)-2- fluorobenzyl)-6,7- dihydro-5H-pyrrolo[3,4- b]pyridin-5-one-7,7-d₂ | ES-MS [M + H]⁺ = 389.3. |
| 26 | 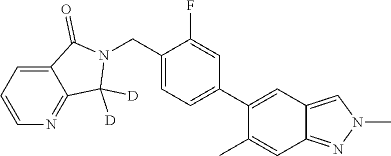 [SMILES: [2H]C1([2H])c2ncccc2C(=O)N1Cc1ccc(-c2cc3cn(C)nc3cc2C)cc1F] | 6-(4-(2,6-dimethyl-2H- indazol-5-yl)-2- fluorobenzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.15 (dd, J = 7.7, 1.6 Hz, 1H), 7.85 (d, J = 1.0 Hz, 1H), 7.54 (q, J = 1.0 Hz, 1H), 7.45-7.35 (m, 3H), 7.14-7.06 (m, 2H), 4.95 (d, J = 1.3 Hz, 2H), 4.21 (s, 3H), 2.29 (d, J = 1.0 Hz, 3H); ES-MS [M + H]⁺ = 389.4. |
| 27 | 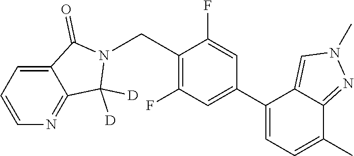 [SMILES: [2H]C1([2H])c2ncccc2C(=O)N1Cc1c(F)cc(-c2ccc(C)c3nn(C)cc23)cc1F] | 6-(4-(2,7-dimethyl-2H- indazol-4-yl)-2,6- difluorobenzyl)-6,7- dihydro-5H-pyrrolo[3,4- b]pyridin-5-one-7,7-d₃ | ¹H NMR (400 MHz, CDCl₃) δ 8.71 (dd, J = 5.0, 1.6 Hz, 1H), 8.14 (dd, J = 7.7, 1.6 Hz, 1H), 8.03 (s, 1H), 7.40 (dd, J = 7.7, 4.9 Hz, 1H), 7.26-7.21 (m, 2H), 7.12 (dd, J = 7.0, 0.8 Hz, 1H), 7.07 (d, J = 7.7 Hz, 1H), 5.00 (s, 2H), 4.27 (s, 3H), 2.66 (s, 3H); ES-MS [M + H]⁺ = 407.3. |
| 28 | 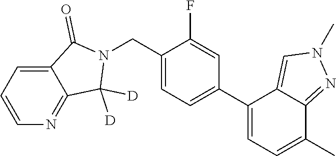 [SMILES: [2H]C1([2H])c2ncccc2C(=O)N1Cc1ccc(-c2ccc(C)c3nn(C)cc23)cc1F] | 6-(4-(2,7-dimethyl-2H- indazol-4-yl)-2- fluorobenzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.15 (dd, J = 7.7, 1.6 Hz, 1H), 8.01 (s, 1H), 7.51-7.34 (m, 4H), 7.11 (dd, J = 7.0, 1.1 Hz, 1H), 7.06 (d, J = 7.0 Hz, 1H), 4.95 (s, 2H), 4.25 (s, 3H), 2.66 (d, J = 1.0 Hz, 3H); ES-MS [M + H]⁺ = 389.4 |
| 29 | 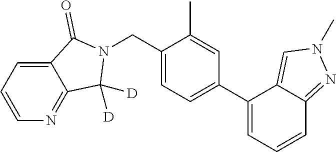 [SMILES: [2H]C1([2H])c2ncccc2C(=O)N1Cc1ccc(-c2cccc3nn(C)cc23)cc1C] | 6-(2-methyl-4-(2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.72 (dd, J = 5.0, 1.6 Hz, 1H), 8.18 (dd, J = 7.7, 1.6 Hz, 1H), 8.01 (s, 1H), 7.68 (d, J = 8.7 Hz, 1H), 7.51-7.47 (m, 2H), 7.42 (dd, J = 7.7, 5.0 Hz, 1H), 7.39-7.29 (m, 2H), 7.14 (dd, J = 6.9, 0.8 Hz, 1H), 4.92 (s, 2H), 4.23 (s, 3H), 2.45 (s, 3H); ES-MS [M + H]⁺ = 371.4. |
| 30 | 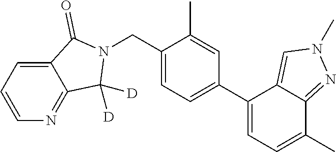 [SMILES: [2H]C1([2H])c2ncccc2C(=O)N1Cc1ccc(-c2ccc(C)c3nn(C)cc23)cc1C] | 6-(4-(2,7-dimethyl-2H- indazol-4-yl)-2- methylbenzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.17 (dd, J = 7.7, 1.6 Hz, 1H), 8.01 (s, 1H), 7.50-7.40 (m, 2H), 7.31- 7.26 (m, 2H), 7.11 (dd, J = 7.0, 1.1 Hz, 1H), 7.06 (d, J = 7.0 Hz, 1H), 4.94 (s, 2H), 4.25 (s, 3H), 2.68 (s, 3H), 2.45 (s, 3H); ES-MS [M + H]⁺ = 385.4. |
| 31 | 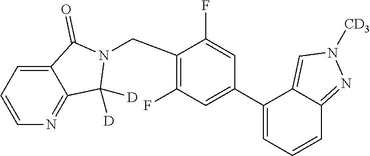 [SMILES: [2H]C1([2H])c2ncccc2C(=O)N1Cc1c(F)cc(-c2cccc3nn(C([2H])([2H])[2H])cc23)cc1F] | 6-(2,6-difluoro-4-(2- (methyl-d₃)-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 5.0, 2.2 Hz, 1H), 8.62 (d, J = 0.8 Hz, 1H), 8.12 (dd, J = 7.7, 3.1 Hz, 1H), 7.66 (d, J = 8.5 Hz, 1H), 7.46-7.55 (m, 3H), 7.33 (dd, J = 8.5, 5.2, 1H), 7.27 (d, J = 3.4, 1H), 4.91 (s, 2H); ES-MS [M + H]⁺ = 396.0. |
| 32 | 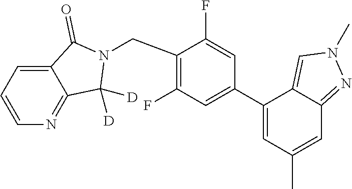 [SMILES: [2H]C1([2H])c2ncccc2C(=O)N1Cc1c(F)cc(-c2cc(C)cc3nn(C)cc23)cc1F] | 6-(4-(2,6-dimethyl-2H- indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 7.96 (s, 1H), 7.48 (d, J = 1.1 Hz, 1H), 7.40 (dd, J = 7.7, 5.0 Hz, 1H), 7.26-7.22 (m, 2H), 7.00 (d, J = 1.3 Hz, 1H), 5.01 (s, 2H), 4.21 (s, 3H), 2.49 (s, 3H); ES-MS [M + H]⁺ = 407.3. |
| 33 | 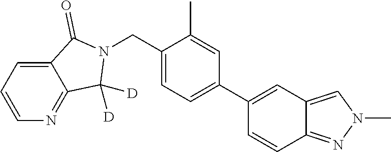 [SMILES: [2H]C1([2H])c2ncccc2C(=O)N1Cc1ccc(-c2ccc3nn(C)cc3c2)cc1C] | 6-(2-methyl-4-(2- methyl-2H-indazol-5- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.16 (dd, J = 7.7, 1.6 Hz, 1H), 7.94 (s, 1H), 7.80 (dd, J = 1.7, 0.9 Hz, 1H), 7.75 (dt, J = 9.0, 0.9 Hz, 1H), 7.54 (dd, J = 9.0, 1.7 Hz, 1H), 7.49-7.38 (m, 3H), 7.29 (d, J = 7.8 Hz, 1H), 4.90 (s, 2H), 4.24 (s, 3H), 2.44 (s, 3H); ES-MS [M + H]⁺ = 371.5. |
| 34 | 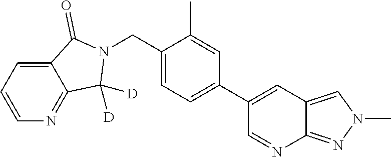 [SMILES: [2H]C1([2H])c2ncccc2C(=O)N1Cc1ccc(-c2cnc3nn(C)cc3c2)cc1C] | 6-(2-methyl-4-(2- methyl-2H-pyrazolo[3,4- b]pyridin-5-yl)benzyl)- 6,7-dihydro-5H- pyrrolo[3,4-b]pyridin-5- one-7,7-d₂ | 1H NMR (400 MHz, CDCl₃) δ 8.94 (d, J = 2.3 Hz, 1H), 8.72 (dd, J = 5.0, 1.6 Hz, 1H), 8.16 (dd, J = 7.7, 1.6 Hz, 1H), 8.13 (d, J = 2.3 Hz, 1H), 7.95 (s, 1H), 7.52-7.38 (m, 3H), 7.33 (d, J = 7.7 Hz, 1H), 4.91 (s, 2H), 4.28 (s, 3H), 2.50 (s, 3H); ES-MS [M + H]⁺ = 372.4. |
| 35 | 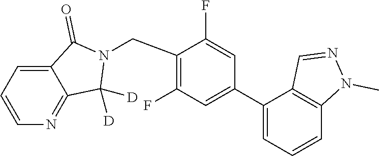 [SMILES: [2H]C1([2H])c2ncccc2C(=O)N1Cc1c(F)cc(-c2cccc3c2cnn3C)cc1F] | 6-(2,6-difluoro-4-(1- methyl-1H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.09 (d, J = 0.8 Hz, 1H), 7.49-7.43 (m, 2H), 7.40 (dd, J = 7.7, 5.0 Hz, 1H), 7.29 (d, J = 8.0 Hz, 2H), 7.20 (dd, J = 6.0, 1.9 Hz, 1H), 5.03 (t, J = 1.3 Hz, 2H), 4.13 (s, 3H); ES-MS [M + H]⁺ = 393.3. |
| 36 | 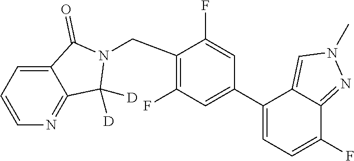 [SMILES: [2H]C1([2H])c2ncccc2C(=O)N1Cc1c(F)cc(-c2ccc(F)c3nn(C)cc23)cc1F] | 6-(2,6-difluoro-4-(7- fluoro-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.14 (dd, J = 7.7, 1.6 Hz, 1H), 8.07 (d, J = 2.7 Hz, 1H), 7.40 (dd, J = 7.7, 4.9 Hz, 1H), 7.25-7.09 (m, 2H), 7.06 (dd, J = 7.7, 4.2 Hz, 1H), 7.00 (dd, J = 10.6, 7.8 Hz, 1H), 5.01 (s, 2H), 4.28 (s, 3H); ES- MS [M + H]⁺ = 411.4. |
| 37 | 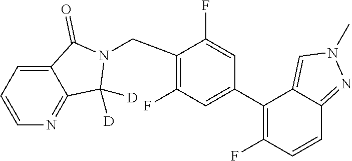 [SMILES: [2H]C1([2H])c2ncccc2C(=O)N1Cc1c(F)cc(-c2c(F)ccc3nn(C)cc23)cc1F] | 6-(2,6-difluoro-4-(5- fluoro-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.15 (dd, J = 7.7, 1.6 Hz, 1H), 7.91 (s, 1H), 7.70 (ddd, J = 9.3, 4.2, 1.0 Hz, 1H), 7.40 (dd, J = 7.7, 4.9 Hz, 1H), 7.26- 7.24 (m, 2H), 7.17 (dd, J = 10.7, 19.3 Hz, 1H), 5.02 (s, 2H), 4.22 (s, 3H); ES-MS [M + H]⁺ = 411.4. |
| 38 | 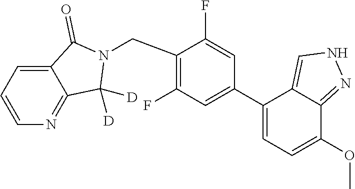 [SMILES: [2H]C1([2H])c2ncccc2C(=O)N1Cc1c(F)cc(-c2ccc(OC)c3n[nH]cc23)cc1F] | 6-(2,6-difluoro-4-(7- methoxy-2H-indazol-4- yl)benzyl)-6,7-dihydro- 5H-pyrrolo[3,4- b]pyridin-5-one-7,7-d₂ | ¹H NMR (400 MHz, CDCl₃) δ 8.72 (dd, J = 5.0, 1.6 Hz, 1H), 8.20 (d, J = 7.5 Hz, 1H), 8.18 (s, 1H), 7.45 (dd, J = 7.7, 5.0 Hz, 1H), 7.26-7.24 (m, 2H), 7.16 (d, J = 7.8 Hz, 1H), 6.81 (d, J = 7.9 Hz, 1H), 5.02 (s, 2H), 4.04 (s, 3H), N—H proton not observable; ES-MS [M + H]⁺ = 409.3. |
| 39 | 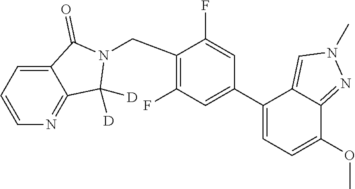 [SMILES: [2H]C1([2H])c2ncccc2C(=O)N1Cc1c(F)cc(-c2ccc(OC)c3nn(C)cc23)cc1F] | 6-(2,6-difluoro-4-(7- methoxy-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 8.02 (s, 1H), 7.40 (dd, J = 7.7, 4.9 Hz, 1H), 7.26-7.16 (m, 2H), 7.09 (d, J = 7.7 Hz, 1H), 6.63 (d, J = 7.8 Hz, 1H), 5.00 (s, 2H), 4.25 (s, 3H), 4.05 (s, 3H); ES-MS [M + H]⁺ = 423.2. |
| 40 | 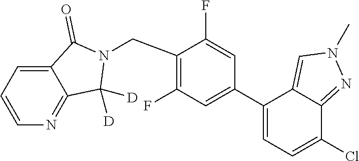 [SMILES: [2H]C1([2H])c2ncccc2C(=O)N1Cc1c(F)cc(-c2ccc(Cl)c3nn(C)cc23)cc1F] | 6-(4-(7-chloro-2-methyl- 2H-indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.14 (dd, J = 7.7, 1.6 Hz, 1H), 8.09 (s, 1H), 7.44-7.35 (m, 2H), 7.25- 7.15 (m, 2H), 7.07 (d, J = 7.5 Hz, 1H), 5.01 (s, 2H), 4.30 (s, 3H); ES-MS [M + H]⁺ = 427.2. |
| 41 | 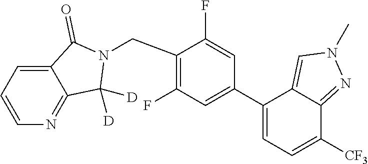 [SMILES: [2H]C1([2H])c2ncccc2C(=O)N1Cc1c(F)cc(-c2ccc(C(F)(F)F)c3nn(C)cc23)cc1F] | 6-(2,6-difluoro-4-(2- methyl-7- (trifluoromethyl)-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.15 (dd, J = 7.7, 1.6 Hz, 1H), 8.12 (s, 1H), 7.68 (dt, J = 7.3, 1.0 Hz, 1H), 7.41 (dd, J = 7.7, 4.9 Hz, 1H), 7.26- 7.22 (m, 2H), 7.17 (dd, J = 7.4, 0.8 Hz, 1H), 5.02 (s, 2H), 4.32 (s, 3H); ES-MS [M + H]⁺ = 461.3. |
| 42 | 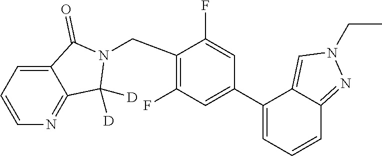 [SMILES: [2H]C1([2H])c2ncccc2C(=O)N1Cc1c(F)cc(-c2cccc3nn(CC)cc23)cc1F] | 6-(4-(2-ethyl-2H- indazol-4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.76 (dd, J = 5.0, 1.6 Hz, 1H), 8.65 (d, J = 1.0 Hz, 1H), 8.13 (dd, J = 7.7, 1.6 Hz, 1H), 7.68 (d, J = 8.5 Hz, 1H), 7.60-7.47 (m, 3H), 7.34 (dd, J = 8.5, 7.0 Hz, 1H), 7.27 (dd, J = 7.0, 0.9 Hz, 1H), 4.92 (s, 2H), 4.48 (q, J = 7.3 Hz, 2H), 1.52 (t, J = 7.3 Hz, 3H); ES-MS [M + H]⁺ = 407.0. |
| 43 | 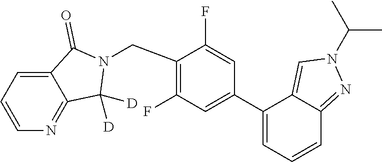 [SMILES: [2H]C1([2H])c2ncccc2C(=O)N1Cc1c(F)cc(-c2cccc3nn(C(C)C)cc23)cc1F] | 6-(2,6-difluoro-4-(1- isopropyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.7 Hz, 1H), 8.14 (dd, J = 7.7, 1.7 Hz, 1H), 8.06 (s, 1H), 7.76 (d, J = 8.1 Hz, 1H), 7.40 (dd, J = 7.7, 5.0 Hz, 1H), 7.35 (dd, 8.7, 7.0 Hz, 1H), 7.29-7.22 (m, 2H), 7.13 (d, J = 6.9 Hz, 1H), 5.01 (s, 2H), 4.82 (hept, J = 6.7 Hz, 1H), 1.67 (d, J = 6.7 Hz, 6H); ES-MS [M + H]⁺ = 421.3. |
| 44 | 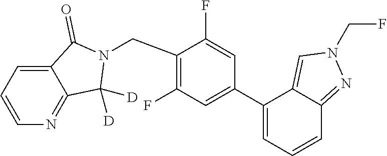 [SMILES: [2H]C1([2H])c2ncccc2C(=O)N1Cc1c(F)cc(-c2cccc3nn(CF)cc23)cc1F] | 6-(2,6-difluoro-4-(2- (fluoromethyl)-2H- indazol-4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.32 (s, 1H), 8.14 (dd, J = 7.7, 1.6 Hz, 1H), 7.78 (d, J = 8.8 Hz, 1H), 7.44-7.36 (m, 2H), 7.29-7.21 (m, 2H), 7.19 (d, J = 6.9 Hz, 1H), 6.33 (d, J = 61.4 Hz, 2H), 5.01 (s, 2H); ES-MS [M + H]⁺ = 411.2. |
| 45 | 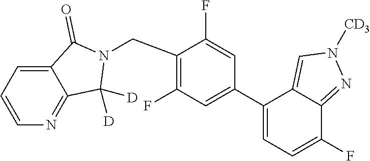 [SMILES: [2H]C1([2H])c2ncccc2C(=O)N1Cc1c(F)cc(-c2ccc(F)c3nn(C([2H])([2H])[2H])cc23)cc1F] | 6-(2,6-difluoro-4-(7- fluoro-2-(methyl-d₃)-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.15 (dd, J = 7.7, 1.6 Hz, 1H), 8.08 (d, J = 2.7 Hz, 1H), 7.41 (dd, J = 7.7, 4.9 Hz, 1H), 7.25-7.14 (m, 2H), 7.09- 6.96 (m, 2H), 5.01 (d, J = 1.5 Hz, 2H); ES- MS [M + H]⁺ = 414.2. |
| 46 | 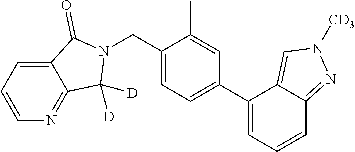 [SMILES: [2H]C1([2H])c2ncccc2C(=O)N1Cc1ccc(-c2cccc3nn(C([2H])([2H])[2H])cc23)cc1C] | 6-(2-methyl-4-(2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.18 (dd, J = 7.7, 1.6 Hz, 1H), 8.02 (s, 1H), 7.69 (dt, J = 8.7, 0.9 Hz, 1H), 751-7.47 (m, 2H), 7.43 (dd, J = 7.7, 5.0 Hz, 1H), 7.36 (dd, J = 8.7, 6.9 Hz, 1H), 7.32 (d, J = 7.5 Hz, 1H), 7.15 (dd, J = 6.9, 0.8 Hz, 1H), 4.93 (s, 2H), 2.46 (s, 3H); ES- MS [M + H]⁺ = 374.2. |
| 47 | 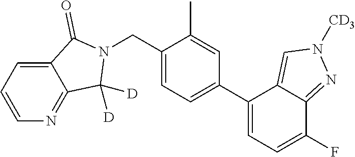 [SMILES: [2H]C1([2H])c2ncccc2C(=O)N1Cc1ccc(-c2ccc(F)c3nn(C([2H])([2H])[2H])cc23)cc1C] | 6-(4-(7-fluoro-2- (methyl-d₃)-2H-indazol- 4-yl)-2-methylbenzyl)- 6,7-dihydro-5H- pyrrolo[3,4-b]pyridin-5- one-7,7-d₂ | ¹H NMR (400 MHz, CDCl₃) δ 8.73 (dd, J = 5.0, 1.5 Hz, 1H), 8.20 (dd, J = 7.7, 1.5 Hz, 1H), 8.04 (d, J = 2.7 Hz, 1H), 7.49-7.36 (m, 3H), 7.31 (d, J = 7.6 Hz, 1H), 7.06-6.92 (m, 2H), 4.92 (s, 2H), 2.44 (s, 3H); ES-MS [M + H]⁺ = 392.2. |
| 48 | 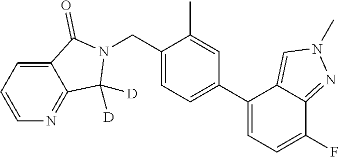 [SMILES: [2H]C1([2H])c2ncccc2C(=O)N1Cc1ccc(-c2ccc(F)c3nn(C)cc23)cc1C] | 6-(4-(7-fluoro-2-methyl- 2H-indazol-4-yl)-2- methylbenzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.20 (dd, J = 7.7, 1.5 Hz, 1H), 8.04 (d, J = 2.7 Hz, 1H), 7.49-7.36 (m, 3H), 7.31 (d, J = 7.7 Hz, 1H), 7.02-6.99 (m, 2H), 4.92 (s, 2H), 4.26 (s, 3H), 2.43 (s, 3H); ES-MS [M + H]⁺ = 389.2. |
| 49 | 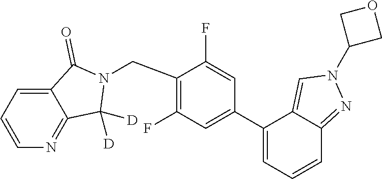 [SMILES: [2H]C1([2H])c2ncccc2C(=O)N1Cc1c(F)cc(-c2cccc3nn(C4COC4)cc23)cc1F] | 6-(2,6-difluoro-4-(2- (oxetan-3-yl)-2H- indazol-4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.21 (d, J = 1.0 Hz, 1H), 81.6 (dd, J = 7.7, 1.6 Hz, 1H), 7.80 (dt, J = 8.7, 0.9 Hz, 1H), 7.46-7.34 (m, 2H), 7.28- 7.23 (m, 2H), 7.18 (dd, J = 6.9, 0.8 Hz, 1H), 5.74 (tt, J = 7.5, 6.0 Hz, 1H), 5.27-5.07 (m, 4H), 5.02 (s, 2H); ES-MS [M + H]⁺ = 435.2. |
| 50 | 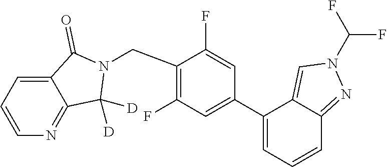 [SMILES: [2H]C1([2H])c2ncccc2C(=O)N1Cc1c(F)cc(-c2cccc3nn(C(F)F)cc23)cc1F] | 6-(4-(2-(difluoromethyl)- 2H-indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.46 (d, J = 1.1 Hz, 1H), 8.15 (dd, J = 7.7, 1.6 Hz, 1H), 7.78 (dt, J = 8.8, 0.9 Hz, 1H), 7.63-7.32 (m, 4H), 7.27- 7.20 (m, 2H), 5.02 (s, 2H); ES-MS [M + H]⁺ = 429.0. |
| 51 | 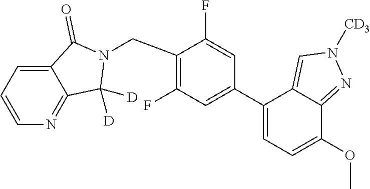 [SMILES: [2H]C1([2H])c2ncccc2C(=O)N1Cc1c(F)cc(-c2ccc(OC)c3nn(C([2H])([2H])[2H])cc23)cc1F] | 6-(2,6-difluoro-4-(7- methoxy-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.71 (dd, J = 5.0, 1.6 Hz, 1H), 8.16 (dd, J = 7.7, 1.6 Hz, 1H), 8.02 (s, 1H), 7.41 (dd, J = 7.7, 5.0 Hz, 1H), 7.25-7.16 (m, 2H), 7.09 (d, J = 7.7 Hz, 1H), 6.63 (d, J = 7.7 Hz, 1H), 5.00 (s, 2H), 4.05 (s, 3H); ES-MS [M + H]⁺ = 426.2. |
| 52 | 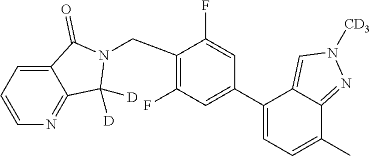 [SMILES: [2H]C1([2H])c2ncccc2C(=O)N1Cc1c(F)cc(-c2ccc(C)c3nn(C([2H])([2H])[2H])cc23)cc1F] | 6-(2,6-difluoro-4-(7- methyl-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.71 (dd, J = 4.9, 1.6 Hz, 1H), 8.15 (dd, J = 7.7, 1.6 Hz, 1H), 8.04 (s, 1H), 7.41 (dd, J = 7.7, 4.9 Hz, 1H), 7.26-7.23 (m, 2H), 7.16-7.02 (m, 2H), 5.01 (s, 2H), 2.67 (s, 3H); ES-MS [M + H]⁺ = 410.2. |
| 53 | 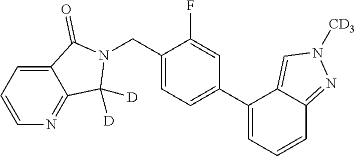 [SMILES: [2H]C1([2H])c2ncccc2C(=O)N1Cc1ccc(-c2cccc3nn(C([2H])([2H])[2H])cc23)cc1F] | 6-(2-fluoro-4-(2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.17 (dd, J = 7.7, 1.6 Hz, 1H), 8.02 (d, J = 1.0 Hz, 1H), 7.71 (dt, J = 8.7, 0.9 Hz, 1H), 7.53-7.30 (m, 5H), 7.15 (dd, J = 6.9, 0.8 Hz, 1H), 4.96 (s, 2H); ES- MS [M + H]⁺ = 378.2. |
| 54 | 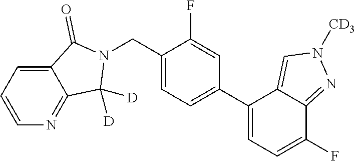 [SMILES: [2H]C1([2H])c2ncccc2C(=O)N1Cc1ccc(-c2ccc(F)c3nn(C([2H])([2H])[2H])cc23)cc1F] | 6-(2-fluoro-4-(7-fluoro- 2-(methyl-d₃)-2H- indazol-4-yl)benzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.18 (dd, J = 7.7, 1.6 Hz, 1H), 8.05 (d, J = 2.7 Hz, 1H), 7.52-7.40 (m, 2H), 7.39-7.33 (m, 2H), 7.04-6.99 (m, 2H), 4.95 (s, 2H); ES-MS [M + H]⁺ = 396.4. |
| 55 | 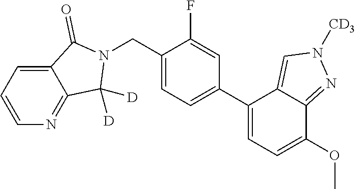 [SMILES: [2H]C1([2H])c2ncccc2C(=O)N1Cc1ccc(-c2ccc(OC)c3nn(C([2H])([2H])[2H])cc23)cc1F] | 6-(2-fluoro-4-(7- methoxy-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.73 (dd, J = 5.0, 1.6 Hz, 1H), 8.18 (dd, J 32 7.7, 1.6 Hz, 1H), 8.00 (s, 1H), 7.51-7.30 (m, 4H), 7.07 (d, J = 7.7 Hz, 1H), 6.63 (d, J = 7.7 Hz, 1H), 4.95 (s, 2H), 4.05 (s, 3H); ES-MS [M + H]⁺ = 408.3. |
| 56 | 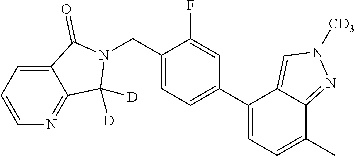 [SMILES: [2H]C1([2H])c2ncccc2C(=O)N1Cc1ccc(-c2ccc(C)c3nn(C([2H])([2H])[2H])cc23)cc1F] | 6-(2-fluoro-4-(7-methyl- 2-(methyl-d₃)-2H- indazol-4-yl)benzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.16 (dd, J = 7.7, 1.6 Hz, 1H), 8.02 (s, 1H), 7.51-7.34 (m, 4H), 7.12 (dd, J = 7.0, 1.2 Hz, 1H), 7.07 (d, J = 7.0 Hz, 1H), 4.95 (s, 2H), 2.66 (s, 3H); ES-MS [M + H]⁺ = 392.4. |
| 57 | 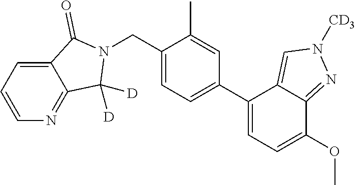 [SMILES: [2H]C1([2H])c2ncccc2C(=O)N1Cc1ccc(-c2ccc(OC)c3nn(C([2H])([2H])[2H])cc23)cc1C] | 6-(4-(7-methoxy-2- (methyl-d₃)-2H-indazol- 4-yl)-2-methylbenzyl)- 6,7-dihydro-5H- pyrrolo[3,4-b]pyridin-5- one-7,7-d₂ | ¹H NMR (400 MHz, CDCl₃) δ 8.73 (dd, J = 5.0, 1.7 Hz, 1H), 8.23 (dd, J = 7.7, 1.6 Hz, 1H), 8.00 (s, 1H), 7.52-7.39 (m, 3H), 7.30 (d, J = 7.6 Hz, 1H), 7.11-7.03 (m, 1H), 6.64 (d, J = 7.7 Hz, 1H), 4.91 (s, 2H), 4.05 (s, 3H), 2.44 (s, 3H); ES-MS [M + H]⁺ = 404.4 |
| 58 | 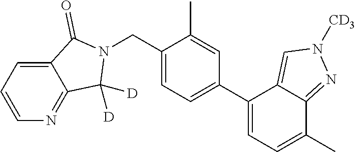 [SMILES: [2H]C1([2H])c2ncccc2C(=O)N1Cc1ccc(-c2ccc(C)c3nn(C([2H])([2H])[2H])cc23)cc1C] | 6-(2-methyl-4-(7- methyl-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.72 (dd, J = 5.0, 1.6 Hz, 1H), 8.19 (dd, J = 7.7, 1.6 Hz, 1H), 8.03 (s, 1H), 7.49-7.36 (m, 3H), 7.31 (d, J = 7.7 Hz, 1H), 7.17 (d, J = 7.0 Hz, 1H), 7.09 (d, J = 7.0 Hz, 1H), 4.91 (s, 2H), 2.68 (s, 3H), 2.43 (s, 3H); ES-MS [M + H]⁺ = 388.4. |
| 59 | 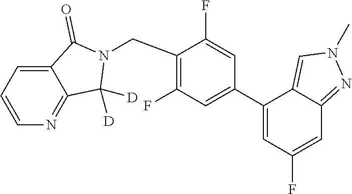 [SMILES: [2H]C1([2H])c2ncccc2C(=O)N1Cc1c(F)cc(-c2cc(F)cc3nn(C)cc23)cc1F] | 6-(2,6-difluoro-4-(6- fluoro-2-methyl-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.76 (dd, J = 4.9, 1.6 Hz, 1H), 8.68 (s, 1H), 8.13 (dd, J = 7.7, 1.6 Hz, 1H), 7.61-7.50 (m, 3H), 7.41 (ddd, J = 9.9, 2.2, 1.0 Hz, 1H), 7.23 (dd, J = 10.4, 2.1 Hz, 1H), 4.92 (s, 2H), 4.16 (s, 3H); ES-MS [M + H]⁺ = 411.0. |
| 60 | 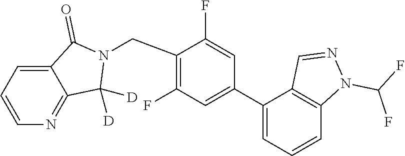 [SMILES: [2H]C1([2H])c2ncccc2C(=O)N1Cc1c(F)cc(-c2cccc3c2cnn3C(F)F)cc1F] | 6-(4-(1-(difluoromethyl) 1H-indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.75-8.69 (m, 1H), 8.20 (d, J = 1.0 Hz, 1H), 8.15 (dd, J = 7.7, 1.3 Hz, 1H), 7.83 (dt, J = 8.4, 0.9 Hz, 1H), 7.59 (dd, J = 8.5, 7.3 Hz, 1H), 7.50 (t, J = 59.3 Hz, 1H), 7.41 (dd, J = 7.7, 4.9 Hz, 1H), 7.34 (d, J = 7.3 Hz, 1H), 7.26-7.22 (m, 2H), 5.03 (s, 2H); ES-MS [M + H]⁺ = 429.0. |
| 61 | 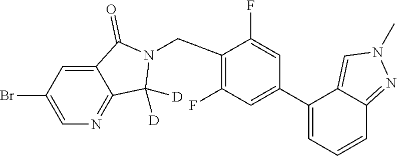 [SMILES: [2H]C1([2H])c2ncc(Br)cc2C(=O)N1Cc1c(F)cc(-c2cccc3nn(C)cc23)cc1F] | 3-bromo-6-(2,6-difluoro- 4-(2-methyl-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.91 (d, J = 2.2 Hz, 1H), 8.63 (d, J = 0.9 Hz, 1H), 8.37 (d, J = 2.2 Hz, 1H), 7.67 (d, J = 8.5 Hz, 1H), 7.53-7.47 (m, 2H), 7.34 (dd, J = 8.5, 7.0 Hz, 1H), 7.28 (dd, J = 6.9, 0.9 Hz, 1H), 4.91 (s, 2H), 4.19 (s, 3H); ES-MS [M + H]⁺ = 473.2. |
| 62 | 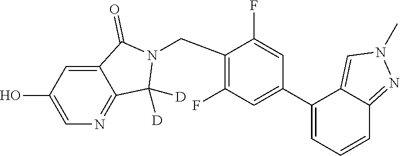 [SMILES: [2H]C1([2H])c2ncc(O)cc2C(=O)N1Cc1c(F)cc(-c2cccc3nn(C)cc23)cc1F] | 6-(2,6-difluoro-4-(2- methyl-2H-indazol-4- yl)benzyl)-3-hydroxy- 6,7-dihydro-5H- pyrrolo[3,4-b]pyridin-5- one-7,7-d₂ | ES-MS [M + H]⁺ = 409.3. |
| 63 | 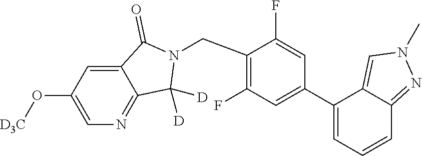 [SMILES: [2H]C([2H])([2H])Oc1cnc2c(c1)C(=O)N(Cc1c(F)cc(-c3cccc4nn(C)cc34)cc1F)C2([2H])[2H]] | 6-(2,6-difluoro-4-(2- methyl-2H-indazol-4- yl)benzyl)-3-(methoxy- d₃)-6,7-dihydro-5H- pyrrolo[3,4-b]pyridin-5- one-7,7-d₂ | ¹H NMR (400 MHz, DMSO-d₆) δ 8.64 (d, J = 1.0 Hz, 1H), 8.46 (d, J = 2.8 Hz, 1H), 7.70-7.63 (m, 2H), 7.53-7.47 (m, 2H), 7.34 (dd, J = 8.5, 6.9 Hz, 1H), 7.28 (dd, J = 6.9, 0.9 Hz, 1H), 4.92 (s, 2H), 4.19 (s, 3H); ES-MS [M + H]⁺ = 426.1. |
| 64 | 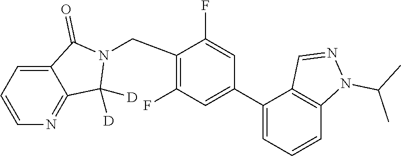 [SMILES: [2H]C1([2H])c2ncccc2C(=O)N1Cc1c(F)cc(-c2cccc3c2cnn3C(C)C)cc1F] | 6-(2,6-difluoro-4-(1- isopropyl-1H-indazol-4- yl)benzyl)-6,7-dihydro- 5H-pyrrolo[3,4- b]pyridin-5-one-7,7-d₂ | ¹H NMR (400 MHz, DMSO-d₆) δ 8.76 (dd, J = 4.9, 1.6 Hz, 1H), 8.23 (s, 1H), 8.12 (dd, J = 7.7, 1.6 Hz, 1H), 7.78 (d, J = 8.5 Hz, 1H), 7.59-7.48 (m, 2H), 7.51-7.43 (m, 2H), 7.31 (dd, J = 7.2, 0.7 Hz, 1H), 5.06 (p, J = 6.6 Hz, 1H), 4.92 (s, 2H), 1.50 (d, J = 6.6 Hz, 6H); ES-MS [M + H]⁺ = 421.0. |
| 65 | 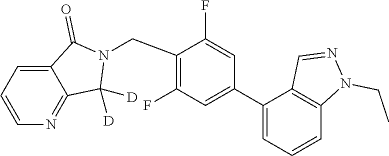 [SMILES: [2H]C1([2H])c2ncccc2C(=O)N1Cc1c(F)cc(-c2cccc3c2cnn3CC)cc1F] | 6-(4-(1-ethyl-1H- indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.71 (dd, J = 4.9, 1.5 Hz, 1H), 8.15 (dd, J = 7.7, 1.4 Hz, 1H), 8.10 (s, 1H), 7.48-7.43 (m, 2H), 7.40 (dd, J = 7.8, 5.1 Hz, 1H), 7.32-7.26 (m, 2H), 7.18 (dd, J = 5.9, 2.0 Hz, 1H), 5.03 (s, 2H), 4.48 (q, J = 7.3 Hz, 2H), 1.54 (t, J = 7.3 Hz, 3H); ES-MS [M + H]⁺ = 407.3. |
| 66 | 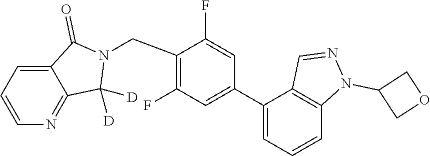 [SMILES: [2H]C1([2H])c2ncccc2C(=O)N1Cc1c(F)cc(-c2cccc3c2cnn3C2COC2)cc1F] | 6-(2,6-difluoro-4-(1- (oxetan-3-yl)-1H- indazol-4-yl)benzyl)-6,7- dihydro-5H-pyrrolo[3,4- b]pyridin-5-one-7,7-d₂ | ¹H NMR (400 MHz, DMSO) δ 8.77 (dd, J = 4.9, 1.6 Hz, 1H), 8.42 (s, 1H), 8.13 (dd, J = 7.7, 1.6 Hz, 1H), 7.79 (d, J = 8.4 Hz, 1H), 7.66-7.48 (m, 4H), 7.41-7.34 (m, 1H), 6.13 (p, J = 7.0 Hz, 1H), 5.12-5.00 (m, 4H), 4.93 (s, 2H); ES-MS [M + H]⁺ = 435.0. |
| 67 | 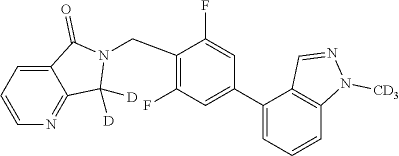 [SMILES: [2H]C1([2H])c2ncccc2C(=O)N1Cc1c(F)cc(-c2cccc3c2cnn3C([2H])([2H])[2H])cc1F] | 6-(2,6-difluoro-4-(1- (methyl-d₃)-1H-indazol- 4-yl)benzyl)-6,7- dihydro-5H-pyrrolo[3,4- b]pyridin-5-one-7,7-d₂ | ¹H NMR (400 MHz, DMSO) δ 8.76 (dd, J = 4.9, 1.6 Hz, 1H), 8.23 (d, J = 1.0 Hz, 1H), 8.13 (dd, J = 7.7, 1.6 Hz, 1H), 7.73 (dt, J = 8.5, 0.9 Hz, 1H), 7.58-7.45 (m, 4H), 7.34 (dd, J = 7.1, 0.8 Hz, 1H), 4.92 (s, 2H); ES- MS [M + H]⁺ = 396.0. |
| 68 | 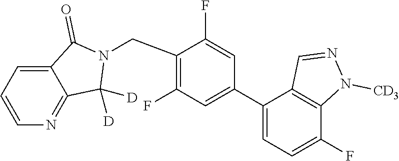 [SMILES: [2H]C1([2H])c2ncccc2C(=O)N1Cc1c(F)cc(-c2ccc(F)c3c2cnn3C([2H])([2H])[2H])cc1F] | 6-(2,6-difluoro-4-(7- fluoro-1-(methyl-d₃)-1H- indazol-4-yl)benzyl)-6,7- dihydro-5H-pyrrolo[3,4- b]pyridin-5-one-7,7-d₂ | ES-MS [M + H]⁺ = 414.0. |
| 69 | 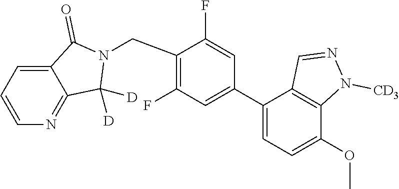 [SMILES: [2H]C1([2H])c2ncccc2C(=O)N1Cc1c(F)cc(-c2ccc(OC)c3c2cnn3C([2H])([2H])[2H])cc1F] | 6-(2,6-difluoro-4-(7- methoxy-1-(methyl-d₃)- 1H-indazol-4-yl)benzyl)- 6,7-dihydro-5H- pyrrolo[3,4-b]pyridin-5- one-7,7-d₂ | ES-MS [M + H]⁺ = 426.0. |
| 70 | 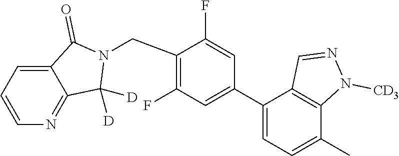 [SMILES: [2H]C1([2H])c2ncccc2C(=O)N1Cc1c(F)cc(-c2ccc(C)c3c2cnn3C([2H])([2H])[2H])cc1F] | 6-(2,6-difluoro-4-(7- methyl-1-(methyl-d₃)- 1H-indazol-4-yl)benzyl)- 6,7-dihydro-5H- pyrrolo[3,4-b]pyridin-5- one-7,7-d₂ | ES-MS [M + H]⁺ = 410.0. |
| 71 | 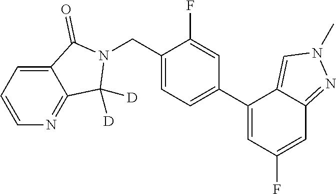 [SMILES: [2H]C1([2H])c2ncccc2C(=O)N1Cc1ccc(-c2cc(F)cc3nn(C)cc23)cc1F] | 6-(2-fluoro-4-(6-fluoro- 2-methyl-2H-indazol-4- yl)benzyl)-6,7-dihydro- 5H-pyrrolo[3,4- b]pyridin-5-one-7,7-d₂ | ES-MS [M + H]⁺ = 393.0. |
| 72 | 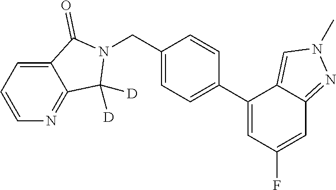 [SMILES: [2H]C1([2H])c2ncccc2C(=O)N1Cc1ccc(-c2cc(F)cc3nn(C)cc23)cc1] | 6-(4-(6-fluoro-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.77 (dd, J = 4.9, 1.6 Hz, 1H), 8.56 (s, 1H), 8.17 (dd, J = 7.7, 1.6 Hz, 1H), 7.78-7.71 (m, 2H), 7.56 (dd, J = 7.7, 4.9 Hz, 1H), 7.51-7.44 (m, 2H), 7.32 (ddd, J = 10.1, 2.1, 0.9 Hz, 1H), 7.08 (dd, J = 10.5, 2.1 Hz, 1H), 4.86 (s, 2H), 4.14 (s, 3H); ES-MS [M + H]⁺ = 375.0. |
| 73 | 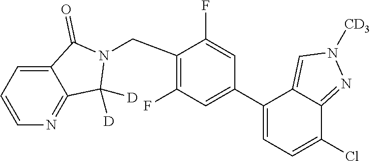 [SMILES: [2H]C1([2H])c2ncccc2C(=O)N1Cc1c(F)cc(-c2ccc(Cl)c3nn(C([2H])([2H])[2H])cc23)cc1F] | 6-(4-(7-chloro-2- (methyl-d₃)-2H-indazol- 4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.79-8.73 (m, 2H), 8.12 (dd, J = 7.7, 1.6 Hz, 1H), 7.58- 7.43 (m, 4H), 7.25 (d, J = 7.6 Hz, 1H), 4.92 (s, 2H); ES-MS [M + H]⁺ = 430.1. |
| 74 | 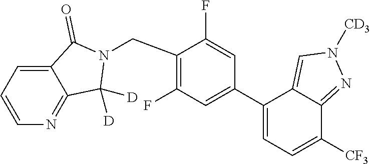 [SMILES: [2H]C1([2H])c2ncccc2C(=O)N1Cc1c(F)cc(-c2ccc(C(F)(F)F)c3nn(C([2H])([2H])[2H])cc23)cc1F] | 6-(2,6-difluoro-4-(2- (methyl-d₃)-7- (trifluoromethyl)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.84 (s, 1H), 8.76 (dd, J = 5.0, 1.6 Hz, 1H), 8.13 (dd, J = 7.7, 1.6 Hz, 1H), 7.75 (dd, J = 7.4, 1.1 Hz, 1H), 7.58-7.51 (m, 3H), 7.37 (d, J = 7.3 Hz, 1H) 1H), 4.94 (s, 2H); ES-MS [M + H]⁺ = 464.0. |
| 75 | 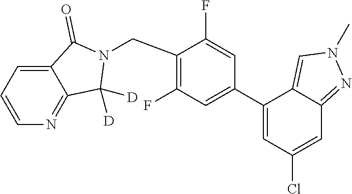 [SMILES: [2H]C1([2H])c2ncccc2C(=O)N1Cc1c(F)cc(-c2cc(Cl)cc3nn(C)cc23)cc1F] | 6-(4-(6-chloro-2-methyl- 2H-indazol-4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.76 (dd, J = 5.0, 1.6 Hz, 1H), 8.68 (s, 1H), 8.12 (dd, J = 7.7, 1.6 Hz, 1H), 7.76 (dd, J = 1.6, 0.9 Hz, 1H), 7.60-7.50 (m, 3H), 7.28 (d, J = 1.7 Hz, 1H), 4.92 (s, 2H), 4.17 (s, 3H); ES-MS [M + H]⁺ = 427.0 |
| 76 | 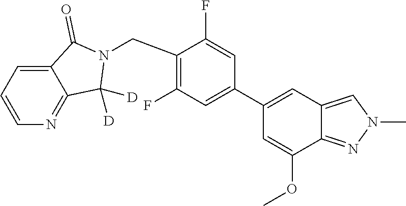 [SMILES: [2H]C1([2H])c2ncccc2C(=O)N1Cc1c(F)cc(-c2cc(OC)c3nn(C)cc3c2)cc1F] | 6-(2,6-difluoro-4-(7- methoxy-2-methyl-2H- indazol-5-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.37 (s, 1H), 8.12 (dd, J = 7.7, 1.6 Hz, 1H), 7.64 (d, J = 1.4 Hz, 1H), 7.61-7.49 (m, 3H), 6.92 (d, J = 1.4 Hz, 1H), 4.88 (s, 2H), 4.15 (s, 3H), 4.00 (s, 3H); ES-MS [M + H]⁺ = 423.0. |
| 77 | 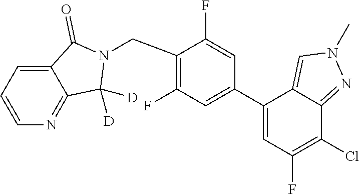 [SMILES: [2H]C1([2H])c2ncccc2C(=O)N1Cc1c(F)cc(-c2cc(F)c(Cl)c3nn(C)cc23)cc1F] | 6-(4-(7-chloro-6-fluoro- 2-methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.82 (d, J = 0.6 Hz, 1H), 8.76 (dd, J = 4.9, 1.6 Hz, 1H), 8.12 (dd, J = 7.7, 1.6 Hz, 1H), 7.61- 7.50 (m, 3H), 7.41 (d, J = 10.7 Hz, 1H), 4.92 (s, 2H), 4.20 (s, 3H); ES-MS [M + H]⁺ = 445.0. |
| 78 | 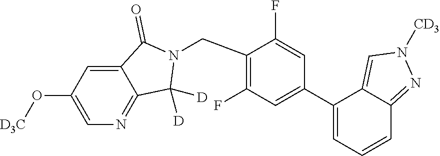 [SMILES: [2H]C([2H])([2H])Oc1cnc2c(c1)C(=O)N(Cc1c(F)cc(-c3cccc4nn(C([2H])([2H])[2H])cc34)cc1F)C2([2H])[2H]] | 6-(2,6-difluoro-4-(2- (methyl-d₃)-2H-indazol- 4-yl)benzyl)-3- (methoxy-d₃)-6,7- dihydro-5H-pyrrolo[3,4- b]pyridin-5-one-7,7-d₂ | ¹H NMR (400 MHz, DMSO-d₆) δ 8.62 (d, J = 1.0 Hz, 1H), 8.45 (d, J = 2.8 Hz, 1H), 7.69-7.62 (m, 2H), 7.54-7.44 (m, 2H), 7.33 (dd, J = 8.5, 6.9 Hz, 1H), 7.27 (dd, J = 7.0, 0.9 Hz, 1H), 4.91 (s, 2H); ES-MS [M + H]⁺ = 429.2. |
| 79 | 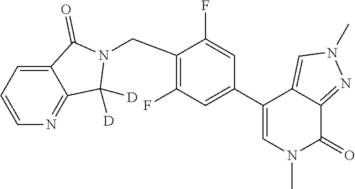 [SMILES: [2H]C1([2H])c2ncccc2C(=O)N1Cc1c(F)cc(-c2cn(C)c(=O)c3nn(C)cc23)cc1F] | 4-(3,5-difluoro-4-((5- oxo-5,7-dihydro-6H- pyrrolo[3,4-b]pyridin-6- yl-7,7- d₂)methyl)phenyl)-2,6- dimethyl-2,6-dihydro- 7H-pyrazolo[3,4- c]pyridin-7-one | ¹H NMR (400 MHz, DMSO) δ 8.75 (dd, J = 4.9, 1.6 Hz, 1H), 8.51 (s, 1H), 8.11 (dd, J = 7.7, 1.6 Hz, 1H), 7.61 (s, 1H), 7.53 (dd, J = 7.7, 4.9 Hz, 1H), 7.42-7.37 (m, 2H), 4.88 (s, 2H), 4.10 (s, 3H), 3.52 (s, 3H); ES-MS [M + H]⁺ = 424.1 |
| 80 | 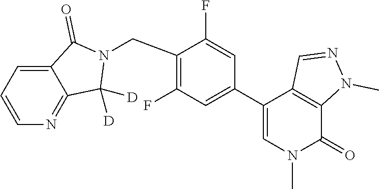 [SMILES: [2H]C1([2H])c2ncccc2C(=O)N1Cc1c(F)cc(-c2cn(C)c(=O)c3c2cnn3C)cc1F] | 4-(3,5-difluoro-4-((5- oxo-5,7-dihydro-6H- pyrrolo[3,4-b]pyridin-6- yl-7,7- d₂)methyl)phenyl)-1,6- dimethyl-1,6-dihydro- 7H-pyrazolo[3,4- c]pyridin-7-one | ¹H NMR (400 MHz, DMSO) δ 8.75 (dd, J = 4.9, 1.6 Hz, 1H), 8.15-8.08 (m, 2H), 7.67 (s, 1H), 7.53 (dd, J = 7.7, 4.9 Hz, 1H), 7.43- 7.38 (m, 2H), 5.75 (s, 1H), 4.88 (s, 2H), 4.30 (s, 3H), 3.57 (s, 3H); ES-MS [M + H]⁺ = 424.1 |
| 81 | 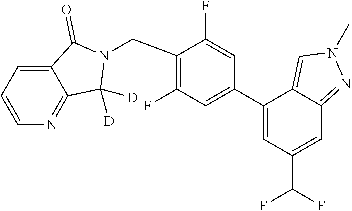 [SMILES: [2H]C1([2H])c2ncccc2C(=O)N1Cc1c(F)cc(-c2cc(C(F)F)cc3nn(C)cc23)cc1F] | 6-(4-(6-(difluoromethyl)- 2-methyl-2H-indazol-4- yl)-2,6-difluorobenzyl)- 6,7-dihydro-5H- pyrrolo[3,4-b]pyridin-5- one-7,7-d₂ | ¹H NMR (400 MHz, CDCl₃) δ 0.71 (dd, J = 5.0, 1.6 Hz, 1H), 8.14 (dd, J = 7.7, 1.6 Hz, 1H), 8.07 (s, 1H), 7.86 (t, J = 1.5 Hz, 1H), 7.41 (dd, J = 7.7, 5.0 Hz, 1H), 7.27 (d, J = 7.9 Hz, 3H), 6.76 (t, J = 56.0 Hz, 1H), 5.02 (s, 2H), 4.27 (s, 3H)); ES-MS [M + H]⁺ = 443.1. |
| 82 | 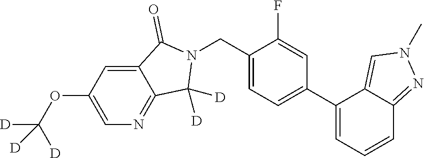 [SMILES: [2H]C([2H])([2H])Oc1cnc2c(c1)C(=O)N(Cc1ccc(-c3cccc4nn(C)cc34)cc1F)C2([2H])[2H]] | 6-(2-fluoro-4-(2-methyl- 2H-indazol-4-yl)benzyl)- 3-(methoxy-d₃)-6,7- dihydro-5H-pyrrolo[3,4- b]pyridin-5-one-7,7-d₂ | ¹H NMR (400 MHz, DMSO) δ 8.56 (s, 1H), 8.47 (d, J = 2.8 Hz, 1H), 7.69 (d, J = 2.8 Hz, 1H), 7.62 (dt, J = 8.6, 0.9 Hz, 1H), 7.60- 7.52 (m, 2H), 7.45 (t, J = 8.1 Hz, 1H), 7.32 (dd, J = 8.6, 6.9 Hz, 1H), 7.20 (dd, J = 7.0, 0.8 Hz, 1H), 4.88 (s, 2H), 4.17 (s, 3H); ES- MS [M + H]⁺ = 408.1 |
| 83 | 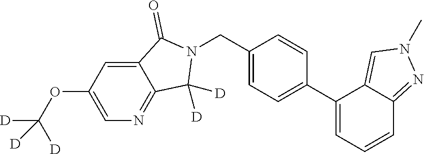 [SMILES: [2H]C([2H])([2H])Oc1cnc2c(c1)C(=O)N(Cc1ccc(-c3cccc4nn(C)cc34)cc1)C2([2H])[2H]] | 3-(methoxy-d₃)-6-(4-(2- methyl-2H-indazol-4- yl)benzyl)-6,7-dihydro- 5H-pyrrolo[3,4- b]pyridin-5-one-7,7-d₂ | ¹H NMR (400 MHz, DMSO) δ 8.49 (s, 1H), 8.47 (d, J = 4.0, 1H), 7.72-7.70 (m, 3H), 7.58 (dt, J = 8.6, 0.9 Hz, 1H), 7.47-7.39 (m, 2H), 7.31 (dd, J = 8.6, 6.9 Hz, 1H), 7.15 (dd, J = 7.0, 0.8 Hz, 1H), 4.84 (s, 2H), 4.16 (s, 3H); ES-MS [M + H]⁺ = 390.1 |
| 84 | 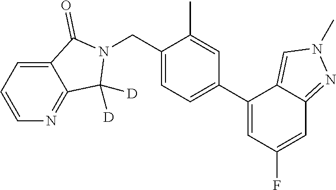 [SMILES: [2H]C1([2H])c2ncccc2C(=O)N1Cc1ccc(-c2cc(F)cc3nn(C)cc23)cc1C] | 6-(4-(6-fluoro-2-methyl- 2H-indazol-4-yl)-2- methylbenzyl)-6,7- dihydro-5H-pyrrolo[3,4- b]pyridin-5-one-7,7-d₂ | ¹H NMR (400 MHz, DMSO) δ 8.77 (dd, J = 5.0, 1.6 Hz, 1H), 8.56 (s, 1H), 8.16 (dd, J = 7.7, 1.6 Hz, 1H), 7.61 (d, J = 1.9 Hz, 1H), 7.59-7.52 (m, 2H), 7.35-7.26 (m, 2H), 7.06 (dd, J = 10.5, 2.2 Hz, 1H), 4.85 (s, 2H), 4.15 (s, 3H), 2.43 (s, 3H); ES-MS [M + H]⁺ = 389.1 |
| 85 | 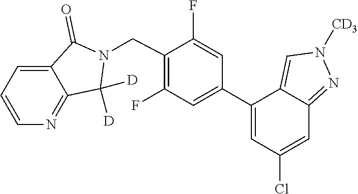 [SMILES: [2H]C1([2H])c2ncccc2C(=O)N1Cc1c(F)cc(-c2cc(Cl)cc3nn(C([2H])([2H])[2H])cc23)cc1F] | 6-(4-(6-chloro-2- (methyl-d₃)-2H-indazol- 4-yl)-2,6- difluorobenzyl)-6,7- dihydro-5H-pyrrolo[3,4- b]pyridin-5-one-7,7-d₂ | ¹H NMR (400 MHz, DMSO) δ 8.76 (dd, J = 4.9, 1.6 Hz, 1H), 8.68 (d, J = 1.0 Hz, 1H), 8.12 (dd, J = 7.7, 1.6 Hz, 1H), 7.76 (dd, J = 1.7, 1.0 Hz, 1H), 7.58-7.50 (m, 3H), 7.28 (d, J = 1.7 Hz, 1H), 4.92 (s, 2H); ES-MS [M + H]⁺ = 430.1 |
| 86 | 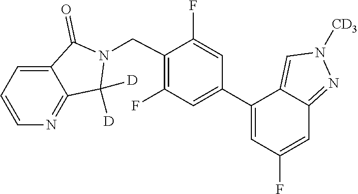 [SMILES: [2H]C1([2H])c2ncccc2C(=O)N1Cc1c(F)cc(-c2cc(F)cc3nn(C([2H])([2H])[2H])cc23)cc1F] | 6-(2,6-difluoro-4-(6- fluoro-2-(methyl-d₃)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.76 (dd, J = 4.9, 1.6 Hz, 1H), 8.67 (s, 1H), 8.12 (dd, J = 7.7, 1.6 Hz, 1H), 7.57-7.52 (m, 3H), 7.41 (dd, J = 10.0, 2.1 Hz, 1H), 7.23 (dd, J = 10.4, 2.1 Hz, 1H), 4.92 (s, 2H); ES-MS [M + H]⁺ = 414.1 |
| 87 | 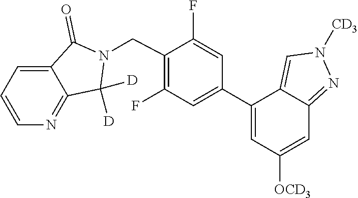 | 6-(2,6-difluoro-4-(6- (methoxy-d₃)-2-(methyl- d₃)-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.48 (d, J = 1.0 Hz, 1H), 8.12 (dd, J = 7.7, 1.6 Hz, 1H), 7.57- 7.46 (m, 3H), 7.00 (dd, J = 2.0, 0.9 Hz, 1H), 6.93 (d, J = 2.1 Hz, 1H), 4.91 (s, 2H); ES- MS [M + H]⁺ = 429.1 |
| 88 | 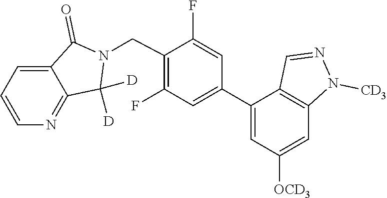 | 6-(2,6-difluoro-4-(6- (methoxy-d₃)-1-(methyl- d₃)-1H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.12 (dd, J = 7.7, 1.6 Hz, 1H), 8.08 (d, J = 1.0 Hz, 1H), 7.57- 7.44 (m, 3H), 7.18 (dd, J = 2.0, 1.0 Hz, 1H), 6.96 (d, J = 2.0 Hz, 1H), 4.91 (s, 2H); ES- MS [M + H]⁺ = 429.1 |
| 89 | 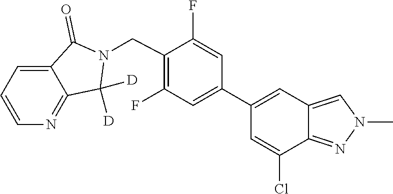 [SMILES: [2H]C1([2H])c2ncccc2C(=O)N1Cc1c(F)cc(-c2cc(Cl)c3nn(C)cc3c2)cc1F] | 6-(4-(7-chloro-2-methyl- 2H-indazol-5-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.57 (s, 1H), 8.15- 8.08 (m, 2H), 7.78 (d, J = 1.5 Hz, 1H), 7.62- 7.56 (m, 2H), 7.53 (dd, J = 7.7, 4.9 Hz, 1H), 4.88 (s, 2H), 4.23 (s, 3H); ES-MS [M + H]⁺ = 427.0 |
| 90 | 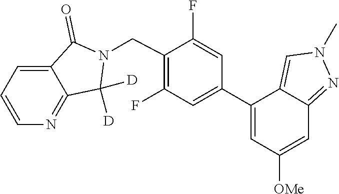 [SMILES: [2H]C1([2H])c2ncccc2C(=O)N1Cc1c(F)cc(-c2cc(OC)cc3nn(C)cc23)cc1F] | 6-(2,6-difluoro-4-(6- methoxy-2-methyl-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.48 (s, 1H), 8.12 (dd, J = 7.7, 1.6 Hz, 1H), 7.57-7.44 (m, 3H), 7.00 (dd, J = 2.0, 0.9 Hz, 1H), 6.93 (d, J = 2.0 Hz, 1H), 4.91 (s, 2H), 4.10 (s, 3H), 3.83 (s, 3H); ES-MS [M + H]⁺ = 423.1 |
| 91 | 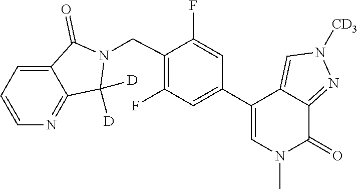 [SMILES: [2H]C1([2H])c2ncccc2C(=O)N1Cc1c(F)cc(-c2cn(C)c(=O)c3nn(C([2H])([2H])[2H])cc23)cc1F] | 4-(3,5-difluoro-4-((5- oxo-5,7-dihydro-6H- pyrrolo[3,4-b]pyridin-6- yl-7,7- d₂)methyl)phenyl)-6- methyl-2-(methyl-d₃)- 2,6-dihydro-7H- pyrazolo[3,4-c]pyridin- 7-one | ¹H NMR (400 MHz, DMSO-d₆) δ 8.75 (dd, J = 4.9, 1.6 Hz, 1H), 8.51 (s, 1H), 8.11 (dd, J = 7.7, 1.6 Hz, 1H), 7.61 (s, 1H), 7.53 (dd, J = 7.7, 4.9 Hz, 1H), 7.46-7.35 (m, 2H), 4.88 (s, 2H), 3.52 (s, 3H); ES-MS [M + H]⁺ = 427.1 |
| 92 | 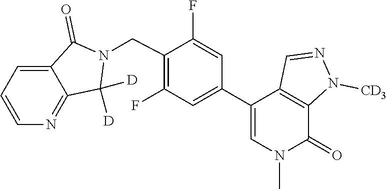 [SMILES: [2H]C1([2H])c2ncccc2C(=O)N1Cc1c(F)cc(-c2cn(C)c(=O)c3c2cnn3C([2H])([2H])[2H])cc1F] | 4-(3,5-difluoro-4-((5- oxo-5,7-dihydro-6H- pyrrolo[3,4-b]pyridin-6- yl-7,7- d₂)methyl)phenyl)-6- methyl-1-(methyl-d₃)- 1,6-dihydro-7H- pyrazolo[3,4-c]pyridin- 7-one | ¹H NMR (400 MHz, DMSO-d₆) δ 8.75 (dd, J = 4.9, 1.6 Hz, 1H), 8.15-8.08 (m, 2H), 7.68 (s, 1H), 7.53 (dd, J = 7.7, 4.9 Hz, 1H), 7.44-7.38 (m, 2H), 4.88 (s, 2H), 3.57 (s, 3H); ES-MS [M + H]⁺ = 427.1 |
| 93 | 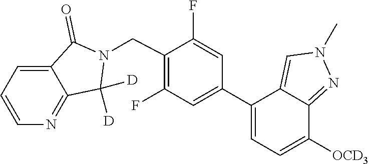 | 6-(2,6-difluoro-4-(7- (methoxy-d₃)-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 5.0 Hz, 1H), 7.44-7.39 (m, 2H), 7.21 (d, J = 7.7 Hz, 1H), 6.70 (d, J = 7.8 Hz, 1H), 4.89 (s, 2H), 4.16 (s, 3H); ES-MS [M + H]⁺ = 426.1 |
| 94 | 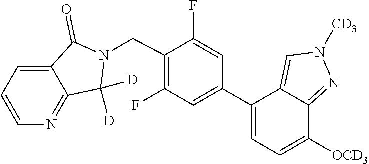 | 6-(2,6-difluoro-4-(7- (methoxy-d₃)-2-(methyl- d₃)-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.47-7.37 (m, 2H), 7.21 (d, J = 7.7 Hz, 1H), 6.70 (d, J = 7.8 Hz, 1H), 4.89 (s, 2H); ES-MS [M + H]⁺ = 419.1 |
| 95 | 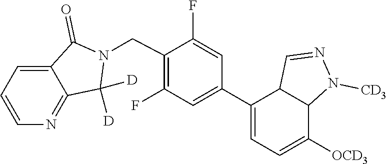 | 6-(2,6-difluoro-4-(7- (methoxy-d₃)-1-(methyl- d₃)-1H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.16 (s, 1H), 8.12 (dd, J = 7.7, 1.6 Hz, 1H), 7.53 (dd, J = 7.7, 4.9 Hz, 1H), 7.46-7.36 (m, 2H), 7.24 (d, J = 7.9 Hz, 1H), 6.93 (d, J = 7.9 Hz, 1H), 4.90 (s, 2H); ES-MS [M + H]⁺ = 429.1 |
| 96 | 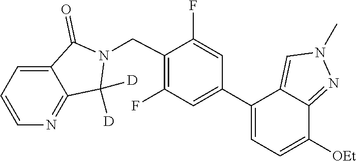 [SMILES: [2H]C1([2H])c2ncccc2C(=O)N1Cc1c(F)cc(-c2ccc(OCC)c3nn(C)cc23)cc1F] | 6-(4-(7-ethoxy-2-methyl- 2H-indazol-4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.44-7.38 (m, 2H), 7.19 (d, J = 7.8 Hz, 1H), 6.69 (d, J = 7.8 Hz, 1H), 4.89 (s, 2H), 4.22 (q, J = 7.0 Hz, 2H), 4.16 (s, 3H), 1.42 (t, J = 7.0 Hz, 3H); ES-MS [M + H]⁺ = 437.1. |
| 97 | 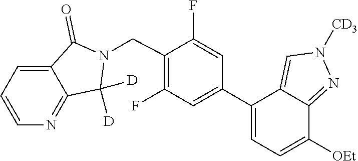 [SMILES: [2H]C1([2H])c2ncccc2C(=O)N1Cc1c(F)cc(-c2ccc(OCC)c3nn(C([2H])([2H])[2H])cc23)cc1F] | 6-(4-(7-ethoxy-2- (methyl-d₃)-2H-indazol- 4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.46-7.36 (m, 2H), 7.19 (d, J = 7.8 Hz, 1H), 6.69 (d, J = 7.8 Hz, 1H), 4.89 (s, 2H), 4.22 (q, J = 7.0 Hz, 2H), 1.42 (t, J = 7.0 Hz, 3H); ES-MS [M + H]⁺ = 440.1. |
| 98 | 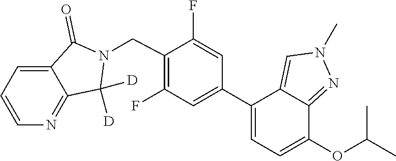 [SMILES: [2H]C1([2H])c2ncccc2C(=O)N1Cc1c(F)cc(-c2ccc(OC(C)C)c3nn(C)cc23)cc1F] | 6-(2,6-difluoro-4-(7- isopropoxy-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.58 (s, 1H), 8.12 (dd, J = 7.7, 1.6 Hz, 1H), 7.53 (dd, J = 7.7, 4.9 Hz, 1H), 7.42-7.38 (m, 2H), 7.18 (d, J = 7.8 Hz, 1H), 6.71 (d, J = 7.9 Hz, 1H), 5.00-4.90 (m, 1H), 4.89 (s, 2H), 4.16 (s, 3H), 1.35 (d, J = 6.0 Hz, 6H); ES-MS [M + H]⁺ = 451.1. |
| 99 | 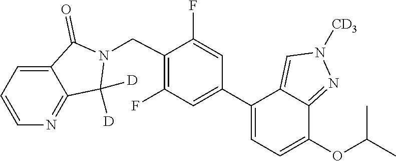 [SMILES: [2H]C1([2H])c2ncccc2C(=O)N1Cc1c(F)cc(-c2ccc(OC(C)C)c3nn(C([2H])([2H])[2H])cc23)cc1F] | 6-(2,6-difluoro-4-(7- isopropoxy-2-(methyl- d₃)-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 5.0, 1.6 Hz, 1H), 8.58 (s, 1H), 8.12 (dd, J = 7.7, 1.6 Hz, 1H), 7.53 (dd, J = 7.7, 4.9 Hz, 1H), 7.46-7.36 (m, 2H), 7.18 (d, J = 7.7 Hz, 1H), 6.71 (d, J = 7.9 Hz, 1H), 5.00- 4.91 (m, 1H), 4.89 (s, 2H), 1.35 (d, J = 6.0 Hz, 6H); ES-MS [M + H]⁺ = 454.1 |
| 100 | 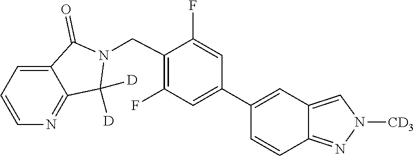 [SMILES: [2H]C1([2H])c2ncccc2C(=O)N1Cc1c(F)cc(-c2ccc3nn(C([2H])([2H])[2H])cc3c2)cc1F] | 6-(2,6-difluoro-4-(2- (methyl-d₃)-2H-indazol- 5-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.43 (d, J = 0.9 Hz, 1H), 8.15-8.08 (m, 2H), 7.67 (dt, J = 9.1, 1.0 Hz, 1H), 7.62 (dd, J = 9.1, 1.8 Hz, 1H), 7.56-7.50 (m, 3H), 4.88 (s, 2H); ES-MS [M + H]⁺ = 395.1 |
| 101 | 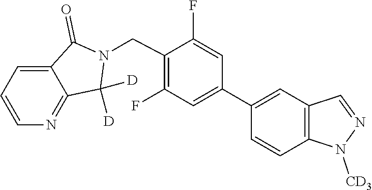 [SMILES: [2H]C1([2H])c2ncccc2C(=O)N1Cc1c(F)cc(-c2ccc3c(cnn3C([2H])([2H])[2H])c2)cc1F] | 6-(2,6-difluoro-4-(1- (methyl-d₃)-1H-indazol- 5-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.43 (d, J = 0.9 Hz, 1H), 8.13-8.10 (m, 2H), 7.80 (dd, J = 9.0, 1.6 Hz, 1H), 7.62 (dt, J = 9.1, 1.8 Hz, 1H), 7.59-7.51 (m, 3H), 4.88 (s, 2H); ES-MS [M + H]⁺ = 395.1 |
| 102 | 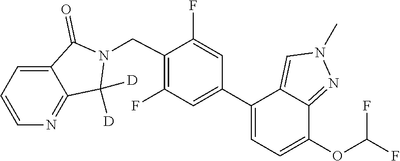 [SMILES: [2H]C1([2H])c2ncccc2C(=O)N1Cc1c(F)cc(-c2ccc(OC(F)F)c3nn(C)cc23)cc1F] | 6-(4-(7- (difluoromethoxy)-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.79-8.74 (m, 2H), 8.12 (dd, J = 7.7, 1.6 Hz, 1H), 7.55 (t, J = 74.4 Hz, 1H), 7.53 (dd, J = 7.7, 5.0 Hz, 1H), 7.50-7.44 (m, 2H), 7.26 (d, J = 7.7 Hz, 1H), 7.08 (d, J = 7.7 Hz, 1H), 4.91 (s, 2H), 4.21 (s, 3H); ES-MS [M + H]⁺ = 459.1 |
| 103 | 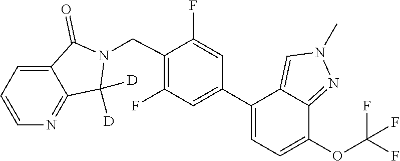 [SMILES: [2H]C1([2H])c2ncccc2C(=O)N1Cc1c(F)cc(-c2ccc(OC(F)(F)F)c3nn(C)cc23)cc1F] | 6-(2,6-difluoro-4-(2- methyl-7- (trifluoromethoxy)-2H- indazol-4-yl)benzyl)-6,7- dihydro-5H-pyrrolo[3,4- b]pyridin-5-one-7,7-d₂ | ES-MS [M + H]⁺ = 477.1 |
| 104 | 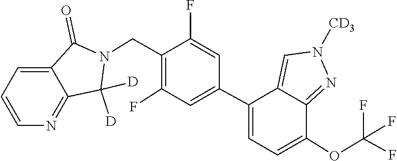 [SMILES: [2H]C1([2H])c2ncccc2C(=O)N1Cc1c(F)cc(-c2ccc(OC(F)(F)F)c3nn(C([2H])([2H])[2H])cc23)cc1F] | 6-(2,6-difluoro-4-(2- (methyl-d₃)-7- (trifluoromethoxy)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.79 (s, 1H), 8.76 (dd, J = 4.9, 1.6 Hz, 1H), 7.57- 7.46 (m, 3H), 7.34 (dd, J = 7.8, 1.5 Hz, 1H), 7.29 (d, J = 7.8 Hz, 2H), 4.92 (s, 2H); ES- MS [M + H]⁺ = 480.1 |
| 105 | 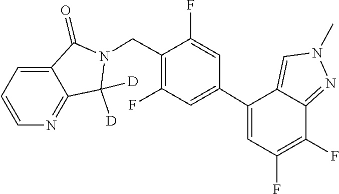 [SMILES: [2H]C1([2H])c2ncccc2C(=O)N1Cc1c(F)cc(-c2cc(F)c(F)c3nn(C)cc23)cc1F] | 6-(4-(6,7-difluoro-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.82 (d, J = 2.6 Hz, 1H), 8.76 (dd, J = 4.9, 1.6 Hz, 1H), 8.12 (dd, J = 7.7, 1.6 Hz, 1H), 7.58-7.48 (m, 3H), 7.41 (dd, J = 12.0, 6.4 Hz, 1H), 4.91 (s, 2H), 4.20 (s, 3H); ES-MS [M + H]⁺ = 429.1 |
| 106 | 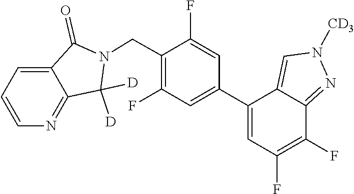 [SMILES: [2H]C1([2H])c2ncccc2C(=O)N1Cc1c(F)cc(-c2cc(F)c(F)c3nn(C([2H])([2H])[2H])cc23)cc1F] | 6-(4-(6,7-difluoro-2- (methyl-d₃)-2H-indazol- 4-yl)-2,6- difluorobenzyl)-6,7- dihydro-5H-pyrrolo[3,4- b]pyridin-5-one-7,7-d₂ | ¹H NMR (400 MHz, DMSO-d₆) δ 8.81 (d, J = 2.7 Hz, 1H), 8.76 (dd, J = 4.9, 1.6 Hz, 1H), 8.12 (dd, J = 7.7, 1.6 Hz, 1H), 7.55- 7.50 (m, 3H), 7.41 (dd, J = 12.0, 6.4 Hz, 1H), 4.91 (s, 2H); ES-MS [M + H]⁺ = 432.2 |
| 107 | 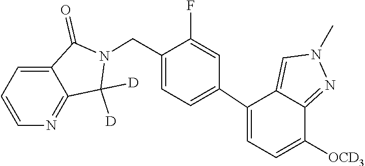 | 6-(2-fluoro-4-(7- (methoxy-d₃)-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.77 (dd, J = 5.0, 1.6 Hz, 1H), 8.52 (s, 1H), 8.15 (dd, J = 7.7, 1.6 Hz 1H), 7.55 (dd, J = 7.7, 4.9 Hz, 1H), 7.52-7.46 (m, 2H), 7.42 (t, J = 7.9 Hz, 1H), 7.13 (d, J = 7.7 Hz, 1H), 6.69 (d, J = 7.7 Hz, 1H), 4.87 (s, 2H), 4.15 (s, 3H); ES-MS [M + H]⁺ = 408.2 |
| 108 | 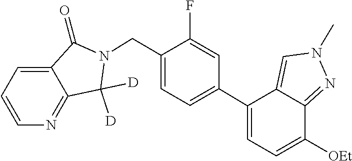 [SMILES: [2H]C1([2H])c2ncccc2C(=O)N1Cc1ccc(-c2ccc(OCC)c3nn(C)cc23)cc1F] | 6-(4-(7-ethoxy-2-methyl- 2H-indazol-4-yl)-2- fluorobenzyl)-6,7- dihydro-5H-pyrrolo[3,4- b]pyridin-5-one-7,7-d₂ | ¹H NM (400 MHz, DMSO-d₆) δ 8.77 (dd, J = 4.9, 1.6 Hz, 1H), 8.15 (dd, J = 7.7, 1.6 Hz, 1H), 7.55 (dd, J = 7.7, 5.0 Hz, 1H), 7.53- 7.45 (m, 2H), 7.42 (t, J = 7.9 Hz, 1H), 7.11 (d, J = 7.7 Hz, 1H), 6.68 (d, J = 7.8 Hz, 1H), 4.87 (s, 2H), 4.21 (q, J = 7.0 Hz, 2H), 4.15 (s, 3H), 1.42 (t, J = 7.0 Hz, 3H); ES-MS [M + H]⁺ = 419.2 |
| 109 | 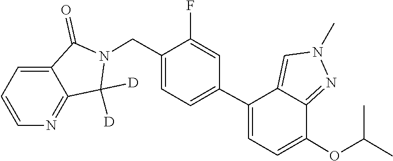 [SMILES: [2H]C1([2H])c2ncccc2C(=O)N1Cc1ccc(-c2ccc(OC(C)C)c3nn(C)cc23)cc1F] | 6-(2-fluoro-4-(7- isopropoxy-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.77 (dd, J = 4.9, 1.6 Hz, 1H), 8.51 (s, 1H), 8.15 (dd, J = 7.7, 1.6 Hz, 1H), 7.55 (dd, J = 7.7, 5.0 Hz, 1H), 7.52-7.45 (m, 2H), 7.42 (t, J = 7.9 Hz, 1H), 7.11 (d, J = 7.7 Hz, 1H), 6.70 (d, J = 7.9 Hz, 1H), 4.92 (p, J = 6.0 Hz, 1H), 4.87 (s, 2H), 4.15 (s, 3H), 1.35 (d, J = 6.0 Hz, 6H); ES-MS [M + H]⁺ = 433.2 |
| 110 | 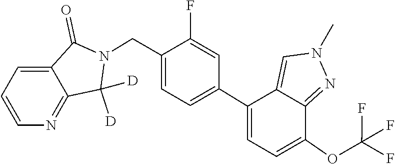 [SMILES: [2H]C1([2H])c2ncccc2C(=O)N1Cc1ccc(-c2ccc(OC(F)(F)F)c3nn(C)cc23)cc1F] | 6-(2-fluoro-4-(2-methyl- 7-(trifluoromethoxy)- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.78 (dd, J = 5.0, 1.6 Hz, 1H), 8.73 (s, 1H), 8.15 (dd, J = 7.7, 1.6 Hz, 1H), 7.62-7.52 (m, 3H), 7.48 (t, J = 7.9 Hz, 1H), 7.33 (dq, J = 7.7, 1.4 Hz, 1H), 7.23 (d, J = 7.7 Hz, 1H), 4.90 (s, 2H), 4.22 (s, 3H); ES-MS [M + H]⁺ = 459.1 |
| 111 | 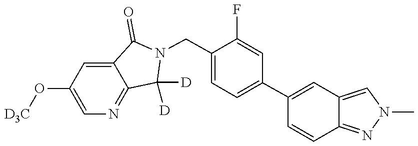 [SMILES: [2H]C([2H])([2H])Oc1cnc2c(c1)C(=O)N(Cc1ccc(-c3ccc4nn(C)cc4c3)cc1F)C2([2H])[2H]] | 6-(2-fluoro-4-(2-methyl- 2H-indazol-5-yl)benzyl)- 3-(methoxy-d₃)-6,7- dihydro-5H-pyrrolo[3,4- b]pyridin-5-one-7,7-d₂ | ¹H NMR (400 MHz, DMSO-d₆) δ 8.46 (d, J = 2.8 Hz, 1H), 8.40 (s, 1H), 8.02 (dd, J = 1.8, 0.9 Hz, 1H), 7.68 (d, J = 2.8 Hz, 1H), 7.68-7.65 (m, 1H), 7.60-7.55 (m, 2H), 7.54 (dd, J = 7.9, 1.8 Hz, 1H), 7.39 (t, J = 8.0 Hz, 1H), 4.85 (s, 2H), 4.18 (s, 3H); ES-MS [M + H]⁺ = 408.2 |
| 112 | 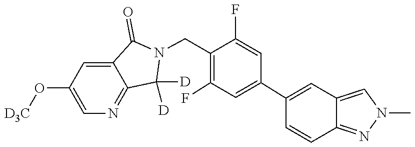 [SMILES: [2H]C([2H])([2H])Oc1cnc2c(c1)C(=O)N(Cc1c(F)cc(-c3ccc4nn(C)cc4c3)cc1F)C2([2H])[2H]] | 6-(2,6-difluoro-4-(2- methyl-2H-indazol-5- yl)benzyl)-3-(methoxy- d₃)-6,7-dihydro-5H- pyrrolo[3,4-b]pyridin-5- one-7,7-d₂ | 1H NMR (400 MHz, DMSO-d₆) δ 8.45 (d, J = 2.8 Hz, 1H), 8.43 (s, 1H), 8.12 (dd, J = 1.8, 0.9 Hz, 1H), 7.67 (dt, J = 9.0, 0.9 Hz, 1H), 7.65 (d, J = 2.8 Hz, 1H), 7.62 (dd, J = 9.1, 1.8 Hz, 1H), 7.52 (d, J = 9.1 Hz, 2H), 4.87 (s, 2H), 4.19 (s, 3H); ES-MS [M + H]⁺ = 426.1 |
| 113 | 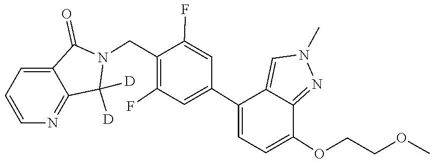 [SMILES: [2H]C1([2H])c2ncccc2C(=O)N1Cc1c(F)cc(-c2ccc(OCCOC)c3nn(C)cc23)cc1F] | 6-(2,6-difluoro-4-(7-(2- methoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 4.9 Hz, 1H), 7.44-4.39 (m, 2H), 7.19 (d, J = 7.8 Hz, 1H), 6.72 (d, J = 7.9 Hz, 1H), 4.89 (s, 2H), 4.33-4.26 (m, 2H), 4.17 (s, 3H), 3.79-3.72 (m, 2H), 3.35 (s, 3H); ES-MS [M + H]⁺ = 467.2 |
| 114 | 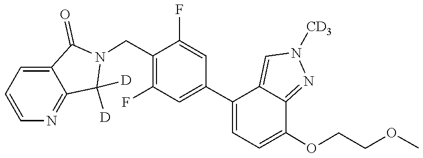 [SMILES: [2H]C1([2H])c2ncccc2C(=O)N1Cc1c(F)cc(-c2ccc(OCCOC)c3nn(C([2H])([2H])[2H])cc23)cc1F] | 6-(2,6-difluoro-4-(7-(2- methoxyethoxy)-2- (methyl-d₃)-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 4.9 Hz, 1H), 7.50-7.37 (m, 2H), 7.19 (d, J = 7.8 Hz, 1H), 6.72 (d, J = 7.9 Hz, 1H), 4.89 (s, 2H), 4.33-4.25 (m, 2H), 3.79-3.72 (m, 2H), 3.35 (s, 3H); ES-MS [M + H]⁺ = 470.1. |
| 115 | 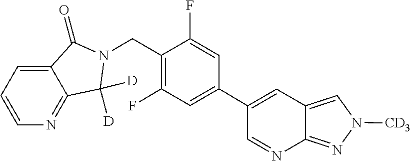 [SMILES: [2H]C1([2H])c2ncccc2C(=O)N1Cc1c(F)cc(-c2cnc3nn(C([2H])([2H])[2H])cc3c2)cc1F] | 6-(2,6-difluoro-4-(2- (methyl-d₃)-2H- pyrazolo[3,4-b]pyridin- 5-yl)benzyl)-6,7- dihydro-5H-pyrrolo[3,4- b]pyridin-5-one-7,7-d₂ | ¹H NMR (400 MHz, DMSO-d₆) δ 8.97 (d, J = 2.4 Hz, 1H), 8.75 (dd, J = 4.9, 1.6 Hz, 1H), 8.58 (d, J = 2.4 Hz, 1H), 8.52 (s, 1H), 8.11 (dd, J = 7.7, 1.6 Hz, 1H), 7.64 (d, J = 8.8 Hz, 2H), 7.53 (dd, J = 7.7, 4.9 Hz, 1H), 4.89 (s, 2H); ES-MS [M + H]⁺ = 397.1 |
| 116 | 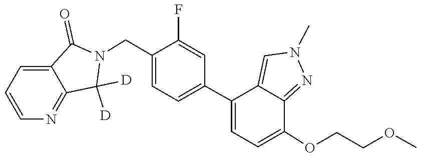 [SMILES: [2H]C1([2H])c2ncccc2C(=O)N1Cc1ccc(-c2ccc(OCCOC)c3nn(C)cc23)cc1F] | 6-(2-fluoro-4-(7-(2- methoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.77 (dd, J = 4.9, 1.6 Hz, 1H), 8.53 (s, 1H), 8.15 (dd, J = 7.7, 1.6 Hz, 1H), 7.55 (dd, J = 7.7, 4.9 Hz, 1H), 7.51-7.45 (m, 2H), 7.42 (t, J = 7.9 Hz, 1H), 7.11 (d, J = 7.7 Hz, 1H), 6.70 (d, J = 7.8 Hz, 1H), 4.87 (s, 2H), 4.32-4.24 (m, 2H), 4.16 (s, 3H), 3.76 (dd, J = 3.9, 2.2 Hz, 2H), 3.35 (s, 3H); ES-MS [M + H]⁺ = 449.2 |
| 117 | 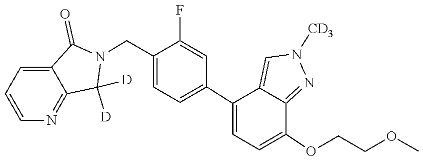 [SMILES: [2H]C1([2H])c2ncccc2C(=O)N1Cc1ccc(-c2ccc(OCCOC)c3nn(C([2H])([2H])[2H])cc23)cc1F] | 6-(2-fluoro-4-(7-(2- methoxyethoxy)-2- (methyl-d₃)-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.77 (dd, J = 4.9, 1.6 Hz, 1H), 8.52 (s, 1H), 8.15 (dd, J = 7.7, 1.6 Hz, 1H), 7.55 (dd, J = 7.7, 4.9 Hz, 1H), 7.51-7.46 (m, 2H), 7.42 (t, J = 7.9 Hz, 1H), 7.11 (d, J = 7.7 Hz, 1H), 6.70 (d, J = 7.8 Hz, 1H), 4.87 (s, 2H), 4.32- 4.25 (m, 2H), 3.79-3.72 (m, 2H), 3.35 (s, 3H); ES-MS [M + H]⁺ = 452.2 |
| 118 | 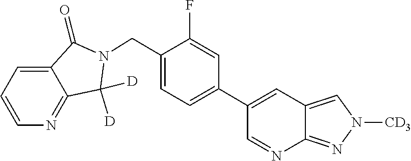 [SMILES: [2H]C1([2H])c2ncccc2C(=O)N1Cc1ccc(-c2cnc3nn(C([2H])([2H])[2H])cc3c2)cc1F] | 6-(2-fluoro-4-(2- (methyl-d₃)-2H- pyrazolo[3,4-b]pyridin- 5-yl)benzyl)-6,7- dihydro-5H-pyrrolo[3,4- b]pyridin-5-one-7,7-d₂ | ¹H NMR (400 MHz, DMSO-d₆) δ 8.93 (d, J = 2.4 Hz, 1H), 8.77 (dd, J = 4.9, 1.6 Hz, 1H), 8.52-8.47 (m, 2H), 8.14 (dd, J = 7.7, 1.6 Hz, 1H), 7.69 (dd, J = 11.6, 1.8 Hz, 1H), 7.60 (dd, J = 8.0, 1.8 Hz, 1H), 7.55 (dd, J = 7.7, 4.9 Hz, 1H), 7.46 (t, J = 8.0 Hz, 1H), 4.87 (s, 2H); ES-MS [M + H]⁺ = 379.2 |
| 119 | 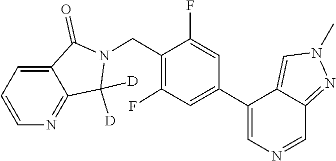 [SMILES: [2H]C1([2H])c2ncccc2C(=O)N1Cc1c(F)cc(-c2cncc3nn(C)cc23)cc1F] | 6-(2,6-difluoro-4-(2- methyl-2H-pyrazolo[3,4- c]pyridin-4-yl)benzyl)- 6,7-dihydro-5H- pyrrolo[3,4-b]pyridin-5- one-7,7-d₂ | ¹H NMR (400 MHz, DMSO-d₆) δ 9.18 (d, J = 0.9 Hz, 1H), 8.84 (d, J = 1.0 Hz, 1H), 8.76 (dd, J = 4.9, 1.6 Hz, 1H), 8.35 (s, 1H), 8.12 (dd, J = 7.7, 1.6 Hz, 1H), 7.62-7.56 (m, 2H), 7.53 (dd, J = 7.7, 4.9 Hz, 1H), 4.92 (s, 2H), 4.28 (s, 3H); ES-MS [M + H]⁺ = 394.1 |
| 120 | 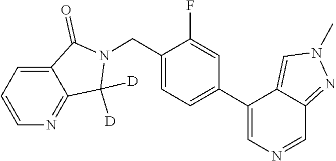 [SMILES: [2H]C1([2H])c2ncccc2C(=O)N1Cc1ccc(-c2cncc3nn(C)cc23)cc1F] | 6-(2-fluoro-4-(2-methyl- 2H-pyrazolo[3,4- c]pyridin-4-yl)benzyl)- 6,7-dihydro-5H- pyrrolo[3,4-b]pyridin-5- one-7,7-d₂ | ¹H NMR (400 MHz, DMSO-d₆) δ 9.15 (d, J = 0.9 Hz, 1H), 8.78-8.77 (m, 2H), 8.29 (s, 1H), 8.15 (dd, J = 7.7, 1.6 Hz, 1H), 7.70- 7.59 (m, 2H), 7.55 (dd, J = 7.7, 5.0 Hz, 1H), 7.50 (t, J = 7.9 Hz, 1H), 4.90 (s, 2H), 4.27 (s, 3H); ES-MS [M + H]⁺ = 376.2 |
| 121 | 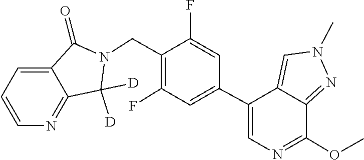 [SMILES: [2H]C1([2H])c2ncccc2C(=O)N1Cc1c(F)cc(-c2cnc(OC)c3nn(C)cc23)cc1F] | 6-(2,6-difluoro-4-(7- methoxy-2-methyl-2H- pyrazolo[3,4-c]pyridin- 4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.72 (s, 1H), 8.12 (dd, J = 7.7, 1.6 Hz, 1H), 7.87 (s, 1H), 7.53 (dd, J = 7.7, 4.9 Hz, 1H), 7.54-7.45 (m, 2H), 4.90 (s, 2H), 4.21 (s, 3H), 4.05 (s, 3H); ES- MS [M + H]⁺ = 424.1 |
| 122 | 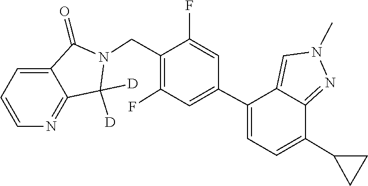 [SMILES: [2H]C1([2H])c2ncccc2C(=O)N1Cc1c(F)cc(-c2ccc(C3CC3)c3nn(C)cc23)cc1F] | 6-(4-(7-cyclopropyl-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 4.9 Hz, 1H), 7.47-7.41 (m, 2H), 7.17 (d, J = 7.3 Hz, 1H), 6.87 (d, J = 7.3 Hz, 1H), 4.90 (s, 2H), 4.19 (s, 3H), 1.06-0.97 (m, 4H); ES-MS [M + H]⁺ = 433.2 |
| 123 | 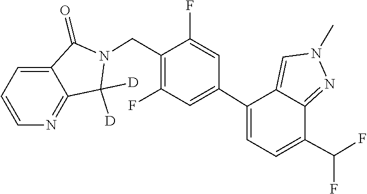 [SMILES: [2H]C1([2H])c2ncccc2C(=O)N1Cc1c(F)cc(-c2ccc(C(F)F)c3nn(C)cc23)cc1F] | 6-(4-(7-(difluoromethyl)- 2-methyl-2H-indazol-4- yl)-2,6-difluorobenzyl)- 6,7-dihydro-5H- pyrrolo[3,4-b]pyridin-5- one-7,7-d₂ | ES-MS [M + H]⁺ = 443.1 |
| 124 | 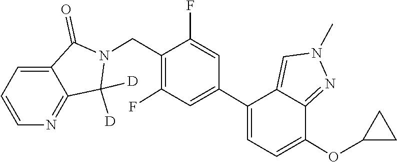 [SMILES: [2H]C1([2H])c2ncccc2C(=O)N1Cc1c(F)cc(-c2ccc(OC3CC3)c3nn(C)cc23)cc1F] | 6-(4-(7-cyclopropoxy-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.44-7.39 (m, 2H), 7.23 (d, J = 7.8 Hz, 1H), 6.98 (d, J = 7.8 Hz, 1H), 4.89 (s, 2H), 4.05 (tt, J = 6.1, 3.0 Hz, 1H), 0.90- 0.81 (m, 2H), 0.81-0.71 (m, 2H); ES-MS [M + H]⁺ = 449.1 |
| 125 | 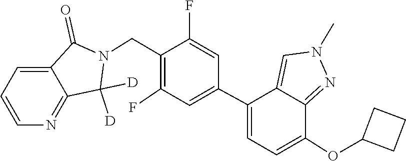 [SMILES: [2H]C1([2H])c2ncccc2C(=O)N1Cc1c(F)cc(-c2ccc(OC3CCC3)c3nn(C)cc23)cc1F] | 6-(4-(7-cyclobutoxy-2- methyl-2H-indazol-4- yl)-2,6-difluorobenzyl)- 6,7-dihydro-5H- pyrrolo[3,4-b]pyridin-5- one-7,7-d₂ | ES-MS [M + H]⁺ = 463.2 |
| 126 | 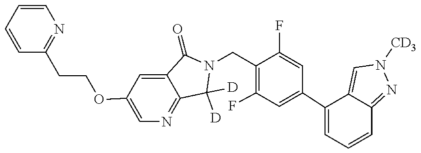 [SMILES: [2H]C1([2H])c2ncc(OCCc3ccccn3)cc2C(=O)N1Cc1c(F)cc(-c2cccc3nn(C([2H])([2H])[2H])cc23)cc1F] | 6-(2,6-difluoro-4-(2- (methyl-d₃)-2H-indazol- 4-yl)benzyl)-3-(2- (pyridin-2-yl)ethoxy)- 6,7-dihydro-5H- pyrrolo[3,4-b]pyridin-5- one-7,7-d₂ | ¹H NMR (400 MHz, DMSO-d₆) δ 8.62 (d, J = 1.0 Hz, 1H), 8.51 (ddd, J = 4.9, 1.9, 0.9 Hz, 1H), 8.40 (d, J = 2.8 Hz, 1H), 7.73 (td, J = 7.6, 1.9 Hz, 1H), 7.70-7.62 (m, 2H), 7.52-7.46 (m, 2H), 7.38 (dt, J = 7.8, 1.1 Hz, 1H), 7.33 (dd, J = 8.5, 7.0 Hz, 1H), 7.29- 7.20 (m, 2H), 4.90 (s, 2H), 4.53 (t, J = 6.6 Hz, 2H), 3.22 (d, J = 13.1 Hz, 2H); ES-MS [M + H]⁺ = 571.2 |
| 127 | 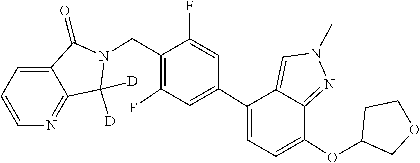 [SMILES: [2H]C1([2H])c2ncccc2C(=O)N1Cc1c(F)cc(-c2ccc(OC3CCOC3)c3nn(C)cc23)cc1F] | 6-(2,6-difluoro-4-(2- methyl-7- ((tetrahydrofuran-3- yl)oxy)-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 4.9 Hz, 1H), 7.45-7.39 (m, 2H), 7.19 (d, J = 7.7 Hz, 1H), 6.69 (d, J = 7.8 Hz, 1H), 5.30 (td, J = 4.6, 2.1 Hz, 1H), 4.89 (s, 2H), 4.16 (s, 3H), 3.97 (dd, J = 10.2, 4.6 Hz, 1H), 3.93- 3.84 (m, 2H), 3.79 (td, J = 8.3, 4.6 Hz, 1H), 2.33-2.22 (m, 1H), 2.08 (dt, J = 12.4, 5.5 Hz, 1H); ES-MS [M + H]⁺ = 479.1 |
| 128 | 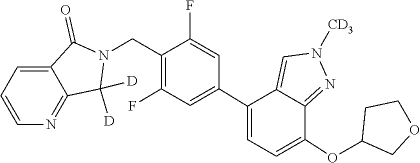 [SMILES: [2H]C1([2H])c2ncccc2C(=O)N1Cc1c(F)cc(-c2ccc(OC3CCOC3)c3nn(C([2H])([2H])[2H])cc23)cc1F] | 6-(2,6-difluoro-4-(2- (methyl-d₃)-7- ((tetrahydrofuran-3- yl)oxy)-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.60 (s, 1H), 8.12 (dd, J = 7.7, 1.6 Hz, 1H), 7.53 (dd, J = 7.7, 4.9 Hz, 1H), 7.45-7.39 (m, 2H), 7.19 (d, J = 7.7 Hz, 1H), 6.69 (d, J = 7.8 Hz, 1H), 5.30 (td, J = 4.6, 2.1 Hz, 1H), 4.89 (s, 2H), 3.97 (dd, J = 10.2, 4.5 Hz, 1H), 3.93-3.84 (m, 2H), 3.79 (td, J = 8.3, 4.6 Hz, 1H), 2.33-2.22 (m, 1H), 2.08 (dt, J = 12.9, 6.0 Hz, 1H); ES-MS [M + H]⁺ = 482.1 |
| 129 | 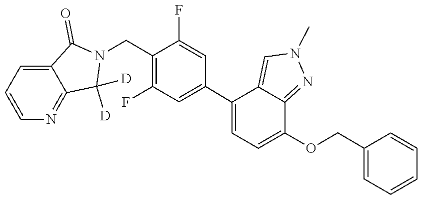 [SMILES: [2H]C1([2H])c2ncccc2C(=O)N1Cc1c(F)cc(-c2ccc(OCc3ccccc3)c3nn(C)cc23)cc1F] | 6-(4-(7-(benzyloxy)-2- methyl-2H-indazol-4- yl)-2,6-difluorobenzyl)- 6,7-dihydro-5H- pyrrolo[3,4-b]pyridin-5- one-7,7-d₂ | 1H NMR (400 MHz, DMSO-d₆) δ 8.75 (dd, J = 4.9, 1.6 Hz, 1H), 8.61 (s, 1H), 8.12 (dd, J = 7.7, 1.6 Hz, 1H), 7.57-7.48 (m, 3H), 7.45-7.33 (m, 5H), 7.20 (d, J = 7.7 Hz, 1H), 6.81 (d, J = 7.9 Hz, 1H), 5.31 (s, 2H), 4.89 (s, 2H), 4.16 (s, 3H); ES-MS [M + H]⁺ = 499.1 |
| 130 | 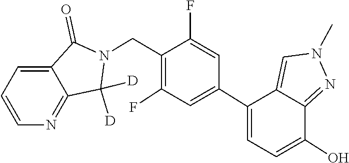 [SMILES: [2H]C1([2H])c2ncccc2C(=O)N1Cc1c(F)cc(-c2ccc(O)c3nn(C)cc23)cc1F] | 6-(2,6-difluoro-4-(7- hydroxy-2-methyl-2H- indazol-4-yl)benzyl)-6,7- dihydro-5H-pyrrolo[3,4- b]pyridin-5-one-7,7-d₂ | ¹H NMR (400 MHz, DMSO-d₆) δ 10.14 (s, 1H), 8.75 (dd, J = 4.9, 1.6 Hz, 1H), 8.57 (s, 1H), 8.12 (dd, J = 7.7, 1.6 Hz, 1H), 7.53 (dd, J = 7.7, 4.9 Hz, 1H), 7.41-7.35 (m, 2H), 7.12 (d, J = 7.6 Hz, 1H), 6.56 (d, J = 7.6 Hz, 1H), 4.88 (s, 2H), 4.17 (s, 3H); ES-MS [M + H]⁺ = 409.0 |
| 131 | 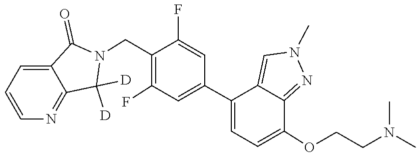 [SMILES: [2H]C1([2H])c2ncccc2C(=O)N1Cc1c(F)cc(-c2ccc(OCCN(C)C)c3nn(C)cc23)cc1F] | 6-(4-(7-(2- (dimethylamino)ethoxy)- 2-methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.44-7.39 (m, 2H), 7.19 (d, J = 7.7 Hz, 1H), 6.73 (d, J = 7.8 Hz, 1H), 4.89 (s, 2H), 4.25 (t, J = 5.8 Hz, 2H), 4.16 (s, 3H), 2.25 (s, 5H); ES-MS [M + H]⁺ = 480.2 |
| 132 | 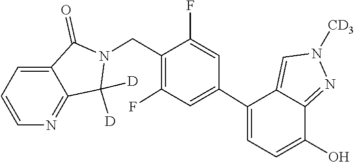 [SMILES: [2H]C1([2H])c2ncccc2C(=O)N1Cc1c(F)cc(-c2ccc(O)c3nn(C([2H])([2H])[2H])cc23)cc1F] | 6-(2,6-difluoro-4-(7- hydroxy-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 10.12 (s, 1H), 8.75 (dd, J = 4.9, 1.6 Hz, 1H), 8.57 (s, 1H), 8.12 (dd, J = 7.7, 1.6 Hz, 1H), 7.53 (dd, J = 7.7, 5.0 Hz, 1H), 7.40-7.35 (m, 2H), 7.12 (d, J = 7.6 Hz, 1H), 6.56 (d, J = 7.6 Hz, 1H), 4.88 (s, 2H); ES-MS [M + H]⁺ = 412.1 |
| 133 | 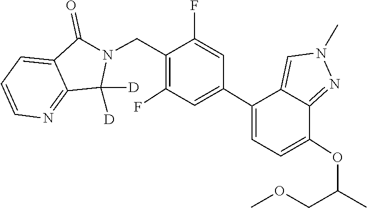 [SMILES: [2H]C1([2H])c2ncccc2C(=O)N1Cc1c(F)cc(-c2ccc(OC(C)COC)c3nn(C)cc23)cc1F] | 6-(2,6-difluoro-4-(7-((1- methoxypropan-2- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.18 (dd, J = 7.7, 1.5 Hz, 1H), 8.01 (s, 1H), 7.44 (dd, J = 7.7, 5.0 Hz, 1H), 7.25-7.15 (m, 2H), 7.06 (d, J = 7.8 Hz, 1H), 6.71 (d, J = 7.8 Hz, 1H), 5.00 (s, 2H), 4.90 (sextet, J = 6.0 Hz, 1H), 4.25 (s, 3H), 3.81 (dd, J = 10.1, 5.8 Hz, 1H), 3.59 (dd, J = 10.0, 5.2 Hz, 1H), 3.43 (s, 3H), 1.48 (d, J = 6.2 Hz, 3H); ES-MS [M + H]⁺ = 481 |
| 134 | 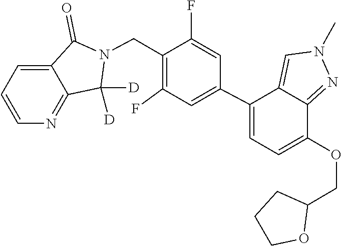 [SMILES: [2H]C1([2H])c2ncccc2C(=O)N1Cc1c(F)cc(-c2ccc(OCC3CCCO3)c3nn(C)cc23)cc1F] | 6-(2,6-difluoro-4-(2- methyl-7- ((tetrahydrofuran-2- yl)methoxy)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.72 (dd, J = 5.1, 1.6 Hz, 1H), 8.22 (dd, J = 7.7, 1.5 Hz, 1H), 8.02 (s, 1H), 7.47 (dd, J = 7.7, 5.0 Hz, 1H), 7.25-7.15 (m, 2H), 7.06 (d, J = 7.7 Hz, 1H), 6.69 (d, J = 7.8 Hz, 1H), 5.00 (s, 2H), 4.52-4.40 (m, 1H), 4.32-4.19 (m, 4H), 4.16 (dd, J = 9.9, 5.6 Hz, 1H), 4.00-3.91 (m, 1H), 3.88-3.78 (m, 1H), 2.24-2.11 (m, 1H), 2.06-1.82 (m, 3H); ES-MS [M + H]⁺ = 493 |
| 135 | 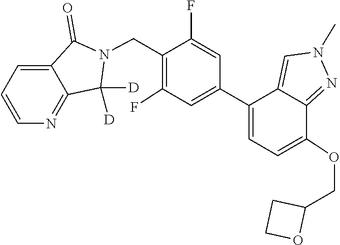 [SMILES: [2H]C1([2H])c2ncccc2C(=O)N1Cc1c(F)cc(-c2ccc(OCC3CCO3)c3nn(C)cc23)cc1F] | 6-(2,6-difluoro-4-(2- methyl-7-(oxetan-2- ylmethoxy)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.19 (dd, J = 7.7, 1.6 Hz, 1H), 8.03 (s, 1H), 7.44 (dd, J = 7.7, 5.0 Hz, 1H), 7.23-7.16 (m, 2H), 7.07 (d, J = 7.7 Hz, 1H), 6.75 (d, J = 7.8 Hz, 1H), 5.34-5.25 (m, 1H), 5.00 (s, 2H), 4.78-4.65 (m, 2H), 4.49- 4.41 (m, 2H), 4.25 (s, 3H), 2.92-2.81 (m, 1H), 2.81-2.70 (m, 1H); ES-MS [M + H]⁺ = 479 |
| 136 | 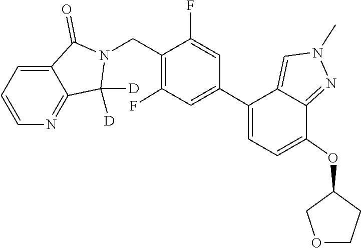 [SMILES: [2H]C1([2H])c2ncccc2C(=O)N1Cc1c(F)cc(-c2ccc(O[C@H]3CCOC3)c3nn(C)cc23)cc1F] | (S)-6-(2,6-difluoro-4-(2- methyl-7- ((tetrahydrofuran-3- yl)oxy)-2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.18 (dd, J = 7.7, 1.6 Hz, 1H), 8.03 (s, 1H), 7.44 (dd, J = 7.7, 5.0 Hz, 1H), 7.23-7.17 (m, 2H), 7.05 (d, J = 7.7 Hz, 1H), 6.56 (d, J = 7.8 Hz, 1H), 5.26-5.20 (m, 1H), 5.00 (s, 2H), 4.26 (s, 3H), 4.20-4.03 (m, 3H), 3.99-3.92 (m, 1H), 2.42-2.25 (m, 2H); ES-MS [M + H]⁺ = 479 |
| 137 | 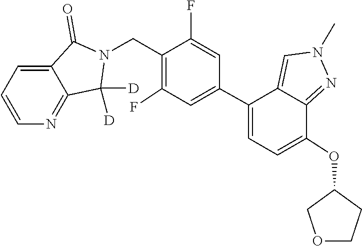 [SMILES: [2H]C1([2H])c2ncccc2C(=O)N1Cc1c(F)cc(-c2ccc(O[C@@H]3CCOC3)c3nn(C)cc23)cc1F] | (R)-6-(2,6-difluoro-4-(2- methyl-7- ((tetrahydrofuran-3- yl)oxy)-2H-indazol-4- yl)benzyl)-6,7-dihydro- 5H-pyrrolo[3,4- b]pyridin-5-one-7,7-d₂ | 1H NMR (400 MHz, CDCl₃) δ 8.71 (dd, J = 5.0, 1.6 Hz, 1H), 8.16 (dd, J = 7.7, 1.6 Hz, 1H), 8.03 (s, 1H), 7.42 (dd, J = 7.7, 5.0 Hz, 1H), 7.23-7.17 (m, 2H), 7.05 (d, J = 7.7 Hz, 1H), 6.56 (d, J = 7.7 Hz, 1H), 5.26-5.20 (m, 1H), 5.00 (s, 2H), 4.26 (s, 3H), 4.21-4.03 (m, 3H), 3.95 (td, J = 8.2, 4.3 Hz, 1H), 2.42- 2.26 (m, 2H); ES-MS [M + H]⁺ = 479 |
| 138 | 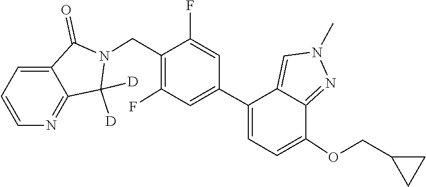 [SMILES: [2H]C1([2H])c2ncccc2C(=O)N1Cc1c(F)cc(-c2ccc(OCC3CC3)c3nn(C)cc23)cc1F] | 6-(4-(7- (cyclopropylmethoxy)-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, DMSO) δ 8.75 (dd, J = 4.9, 1.6 Hz, 1H), 8.59 (s, 1H), 8.12 (dd, J = 7.7, 1.6 Hz, 1H), 7.53 (dd, J = 7.7, 4.9 Hz, 1H), 7.44-7.38 (m, 2H), 7.18 (d, J = 7.8 Hz, 1H), 6.66 (d, J = 7.9 Hz, 1H), 4.89 (s, 2H), 4.17 (s, 3H), 4.01 (d, J = 7.0 Hz, 2H), 1.39-1.25 (m, 1H), 0.67-0.57 (m, 2H), 0.42-0.33 (m, 2H); ES-MS [M + H]⁺ = 463.1 |
| 139 | 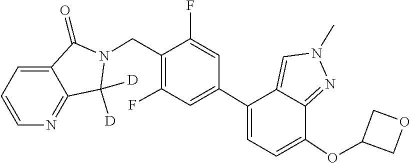 [SMILES: [2H]C1([2H])c2ncccc2C(=O)N1Cc1c(F)cc(-c2ccc(OC3COC3)c3nn(C)cc23)cc1F] | 6-(2,6-difluoro-4-(2- methyl-7-(oxetan-3- yloxy)-2H-indazol-4- yl)benzyl)-6,7-dihydro- 5H-pyrrolo[3,4- b]pyridin-5-one-7,7-d₂ | ¹H NMR (400 MHz, DMSO) δ 8.75 (dd, J = 4.9, 1.6 Hz, 1H), 8.62 (s, 1H), 8.12 (dd, J = 7.7, 1.6 Hz, 1H), 7.53 (dd, J = 7.7, 4.9 Hz, 1H), 7.44-7.38 (m, 2H), 7.14 (d, J = 7.7 Hz, 1H), 6.40 (d, J = 7.7 Hz, 1H), 5.56-5.46 (m, 1H), 5.04-4.96 (m, 2H), 4.89 (s, 2H), 4.68- 4.60 (m, 2H), 4.18 (s, 3H); ES-MS [M + H]⁺ = 465.1 |
| 140 | 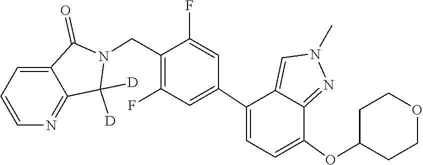 [SMILES: [2H]C1([2H])c2ncccc2C(=O)N1Cc1c(F)cc(-c2ccc(OC3CCOCC3)c3nn(C)cc23)cc1F] | 6-(2,6-difluoro-4-(2- methyl-7-((tetrahydro- 2H-pyran-4-yl)oxy)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 5.0, 1.6 Hz, 1H), 8.60 (s, 1H), 8.12 (dd, J = 7.7, 1.6 Hz, 1H), 7.53 (dd, J = 7.7, 4.9 Hz, 1H), 7.44-7.38 (m, 2H), 7.18 (d, J = 7.8 Hz, 1H), 6.81 (d, J = 7.9 Hz, 1H), 4.95-4.85 (m, 3H), 4.17 (s, 3H), 3.90 (dt, J = 11.7, 4.3 Hz, 2H), 3.52 (ddd, J = 11.8, 9.5, 2.8 Hz, 2H), 2.05 (dd, J = 13.0, 3.7 Hz, 2H), 1.68 (dtd, J = 13.1, 9.2, 4.0 Hz, 2H); ES-MS [M + H]⁺ = 493.1 |
| 141 | 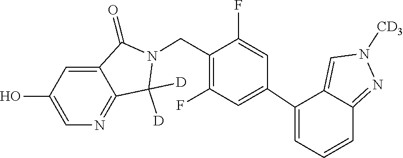 [SMILES: [2H]C1([2H])c2ncc(O)cc2C(=O)N1Cc1c(F)cc(-c2cccc3nn(C([2H])([2H])[2H])cc23)cc1F] | 6-(2,6-difluoro-4-(2- (methyl-d₃)-2H-indazol- 4-yl)benzyl)-3-hydroxy- 6,7-dihydro-5H- pyrrolo[3,4-b]pyridin-5- one-7,7-d₂ | 1H NMR (400 MHz, DMSO-d₆) δ 8.62 (d, J = 0.8 Hz, 1H), 8.27 (d, J = 2.3 Hz, 1H), 7.66 (d, J = 8.5 Hz, 1H), 7.52-7.46 (m, 2H), 7.35-7.26 (m, 3H), 4.88 (s, 2H); ES-MS [M + H]⁺ = 412.1. |
| 142 | 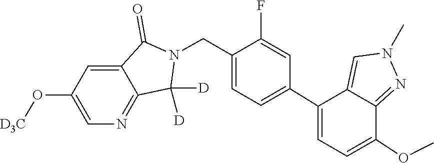 [SMILES: [2H]C([2H])([2H])Oc1cnc2c(c1)C(=O)N(Cc1ccc(-c3ccc(OC)c4nn(C)cc34)cc1F)C2([2H])[2H]] | 6-(2-fluoro-4-(7- methoxy-2-methyl-2H- indazol-4-yl)benzyl)-3- (methoxy-d₃)-6,7- dihydro-5H-pyrrolo[3,4- b]pyridin-5-one-7,7-d₂ | ¹H NMR (400 MHz, DMSO-d₆) δ 8.52 (s, 1H), 8.47 (d, J = 2.8 Hz, 1H), 7.68 (d, J = 2.8 Hz, 1H), 7.50 (s, 1H), 7.47 (dd, 1H), 7.41 (t, J = 7.9 Hz, 1H), 7.13 (d, J = 7.7 Hz, 1H), 6.70 (d, J = 7.8 Hz, 1H), 4.86 (s, 2H), 4.15 (s, 3H), 3.93 (s, 3H); ES-MS [M + H]⁺ = 438.1 |
| 143 | 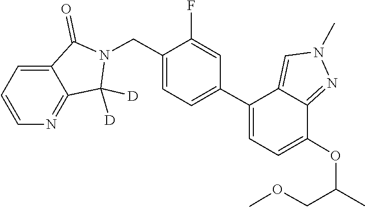 [SMILES: [2H]C1([2H])c2ncccc2C(=O)N1Cc1ccc(-c2ccc(OC(C)COC)c3nn(C)cc23)cc1F] | 6-(2-fluoro-4-(7-((1- methoxypropan-2- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.18 (dd, J = 7.7, 1.6 Hz, 1H), 7.99 (s, 1H), 7.47-7.41 (m, 2H), 7.40- 7.31 (m, 2H), 7.04 (d, J = 7.7 Hz, 1H), 6.71 (d, J = 7.8 Hz, 1H), 4.94 (s, 2H), 4.89 (sextet, J = 6.0 Hz, 1H), 4.24 (s, 3H), 3.81 (dd, J = 10.0, 5.8 Hz, 1H), 3.59 (dd, J = 10.0, 5.3 Hz, 1H), 3.43 (s, 3H), 1.48 (d, J = 6.3 Hz, 3H); ES-MS [M + H]⁺ = 463 |
| 144 | 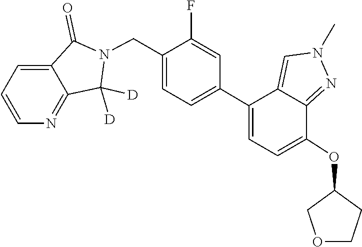 [SMILES: [2H]C1([2H])c2ncccc2C(=O)N1Cc1ccc(-c2ccc(O[C@H]3CCOC3)c3nn(C)cc23)cc1F] | (S)-6-(2-fluoro-4-(2- methyl-7- ((tetrahydrofuran-3- yl)oxy)-2H-indazol-4- yl)benzyl)-6,7-dihydro- 5H-pyrrolo[3,4- b]pyridin-5-one-7,7-d₂ | ¹H NMR (400 MHz, CDCl₃) δ 8.73 (dd, J = 5.0, 1.6 Hz, 1H), 8.18 (dd, J = 7.7, 1.6 Hz, 1H), 8.01 (s, 1H), 7.48-7.41 (m, 2H), 7.40- 7.32 (m, 2H), 7.04 (d, J = 7.7 Hz, 1H), 6.56 (d, J = 7.7 Hz, 1H), 5.26-5.20 (m, 1H), 4.95 (s, 2H), 4.24 (s, 3H), 4.21-4.03 (m, 3H), 3.99-3.91 (m, 1H), 2.42-2.25 (m, 2H); ES- MS [M + H]⁺ = 461 |
| 145 | 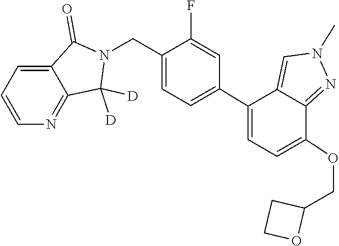 [SMILES: [2H]C1([2H])c2ncccc2C(=O)N1Cc1ccc(-c2ccc(OCC3CCO3)c3nn(C)cc23)cc1F] | 6-(2-fluoro-4-(2-methyl- 7-(oxetan-2-ylmethoxy)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.75-8.69 (m, 1H), 8.18 (dd, J = 7.7, 1.6 Hz, 1H), 8.00 (s, 1H), 7.48-7.41 (m, 2H), 7.40-7.31 (m, 2H), 7.05 (d, J = 7.7 Hz, 1H), 6.75 (d, J = 7.7 Hz, 1H), 5.33-5.24 (m, 1H), 4.94 (s, 2H), 4.77-4.65 (m, 2H), 4.44 (d, J = 4.8 Hz, 2H), 4.24 (s, 3H), 2.91-2.81 (m, 1H), 2.80-2.70 (m, 1H); ES-MS [M + H]⁺ = 461 |
| 146 | 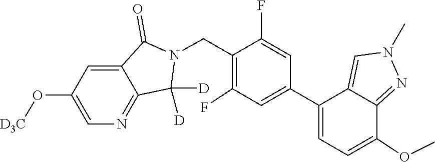 [SMILES: [2H]C([2H])([2H])Oc1cnc2c(c1)C(=O)N(Cc1c(F)cc(-c3ccc(OC)c4nn(C)cc34)cc1F)C2([2H])[2H]] | 6-(2,6-difluoro-4-(7- methoxy-2-methyl-2H- indazol-4-yl)benzyl)-3- (methoxy-d₃)-6,7- dihydro-5H-pyrrolo[3,4- b]pyridin-5-one-7,7-d₂ | ¹H NMR (400 MHz, DMSO-d₆) δ 8.61 (s, 1H), 8.46 (d, J = 2.8 Hz, 1H), 7.66 (d, J = 2.8 Hz, 1H), 7.47-7.37 (m, 2H), 7.22 (d, J = 7.8 Hz, 1H), 6.71 (d, J = 7.8 Hz, 1H), 4.90 (s, 2H), 4.16 (s, 3H), 3.95 (s, 3H); ES-MS [M + H]⁺ = 456.0 |
| 147 | 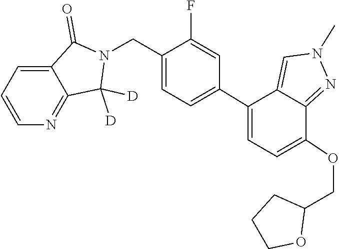 [SMILES: [2H]C1([2H])c2ncccc2C(=O)N1Cc1ccc(-c2ccc(OCC3CCCO3)c3nn(C)cc23)cc1F] | 6-(2-fluoro-4-(2-methyl- 7-((tetrahydrofuran-2- yl)methoxy)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.76-8.70 (m, 1H), 8.21 (d, J = 7.7 Hz, 1H), 8.00 (s, 1H), 7.49-7.41 (m, 2H), 7.40-7.31 (m, 2H), 7.04 (d, J = 7.7 Hz, 1H), 6.69 (d, J = 7.8 Hz, 1H), 4.94 (s, 2H), 4.52-4.41 (m, 1H), 4.32-4.22 (m, 4H), 4.20-4.13 (m, 1H), 4.00-3.92 (m, 1H), 3.88-3.79 (m, 1H), 2.23-2.12 (m, 1H), 2.06-1.84 (m, 3H); ES- MS [M + H]⁺ = 475 |
| 148 | 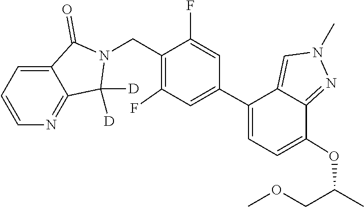 [SMILES: [2H]C1([2H])c2ncccc2C(=O)N1Cc1c(F)cc(-c2ccc(O[C@H](C)COC)c3nn(C)cc23)cc1F] | (R)-6-(2,6-difluoro-4-(7- ((1-methoxypropan-2- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.74-8.68 (m, 1H), 8.15 (dd, J = 7.7, 1.6 Hz, 1H), 8.01 (s, 1H), 7.40 (dd, J = 7.7, 4.9 Hz, 1H), 7.23- 7.16 (m, 2H), 7.06 (d, J = 7.7 Hz, 1H), 6.71 (d, J = 7.8 Hz, 1H), 5.00 (s, 2H), 4.90 (sextet, J = 6.1 Hz, 1H), 4.25 (s, 3H), 3.81 (dd, J = 10.0, 5.8 Hz, 1H), 3.58 (dd, J = 10.0, 5.2 Hz, 1H), 3.43 (s, 3H), 1.48 (d, J = 6.2 Hz, 3H); ES-MS [M + H]⁺ = 481 |
| 149 | 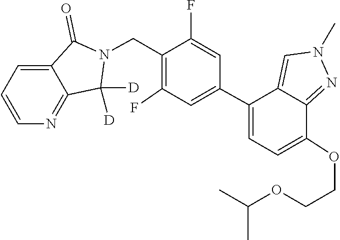 [SMILES: [2H]C1([2H])c2ncccc2C(=O)N1Cc1c(F)cc(-c2ccc(OCCOC(C)C)c3nn(C)cc23)cc1F] | 6-(2,6-difluoro-4-(7-(2- isopropoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.01 (s, 1H), 7.41 (dd, J = 7.7, 4.9 Hz, 1H), 7.23-7.16 (m, 2H), 7.07 (d, J = 7.7 Hz, 1H), 6.69 (d, J = 7.8 Hz, 1H), 5.00 (s, 2H), 4.37 (t, J = 6.0 Hz, 2H), 4.25 (s, 3H), 3.95 (t, J = 6.0 Hz, 2H), 3.72 (septet, J = 6.1 Hz, 1H), 1.20 (d, J = 6.1 Hz, 6H); ES-MS [M + H]⁺ = 495 |
| 150 | 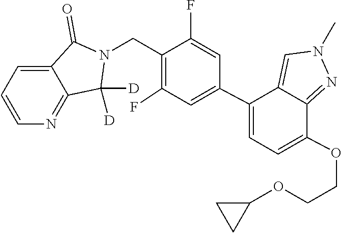 [SMILES: [2H]C1([2H])c2ncccc2C(=O)N1Cc1c(F)cc(-c2ccc(OCCOC3CC3)c3nn(C)cc23)cc1F] | 6-(4-(7-(2- cyclopropoxyethoxy)-2- methyl-2H-indazol-4- yl)-2,6-difluorobenzyl)- 6,7-dihydro-5H- pyrrolo[3,4-b]pyridin-5- one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.01 (s, 1H), 7.41 (dd, J = 7.7, 5.0 Hz, 1H), 7.23-7.16 (m, 2H), 7.06 (d, J = 7.7 Hz, 1H), 6.66 (d, J = 7.8 Hz, 1H), 5.00 (s, 2H), 4.38 (t, J = 5.4 Hz, 2H), 4.25 (s, 3H), 4.02 (t, J = 5.4 Hz, 2H), 3.43 (tt, J = 6.0, 3.0 Hz, 1H), 0.66-0.60 (m, 2H), 0.52-0.45 (m, 2H); ES-MS [M + H]⁺ = 493 |
| 151 | 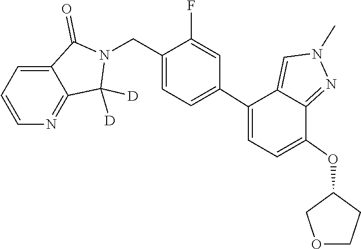 [SMILES: [2H]C1([2H])c2ncccc2C(=O)N1Cc1ccc(-c2ccc(O[C@@H]3CCOC3)c3nn(C)cc23)cc1F] | (R)-6-(2-fluoro-4-(2- methyl-7- ((tetrahydrofuran-3- yl)oxy)-2H-indazol-4- yl)benzyl)-6,7-dihydro- 5H-pyrrolo[3,4- b]pyridin-5-one-7,7-d₂ | ¹H NMR (400 MHz, CDCl₃) δ 8.75-8.69 (m, 1H), 8.16 (dd, J = 7.7, 1.6 Hz, 1H), 8.00 (s, 1H), 7.48-7.40 (m, 2H), 7.40-7.32 (m, 2H), 7.03 (d, J = 7.7 Hz, 1H), 6.56 (d, J = 7.7 Hz, 1H), 5.26-5.19 (m, 1H), 4.94 (s, 2H), 4.24 (s, 3H), 4.21-4.03 (m, 3H), 3.95 (td, J = 8.2, 4.2 Hz, 1H), 2.44-2.22 (m, 2H); ES-MS [M + H]⁺ = 461 |
| 152 | 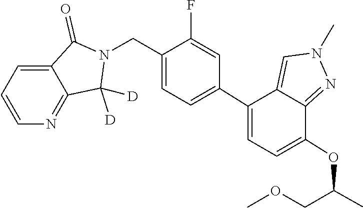 [SMILES: [2H]C1([2H])c2ncccc2C(=O)N1Cc1ccc(-c2ccc(O[C@@H](C)COC)c3nn(C)cc23)cc1F] | (S)-6-(2-fluoro-4-(7-((1- methoxypropan-2- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.75-8.69 (m, 1H), 8.16 (dd, J = 7.7, 1.6 Hz, 1H), 7.99 (s, 1H), 7.47-7.40 (m, 2H), 7.40-7.31 (m, 2H), 7.04 (d, J = 7.7 Hz, 1H), 6.71 (d, J = 7.8 Hz, 1H), 4.94 (s, 2H), 4.89 (sextet, J = 6.0 Hz, 1H), 4.23 (s, 3H), 3.81 (dd, J = 10.0, 5.7 Hz, 1H), 3.58 (dd, J = 10.0, 5.4 Hz, 1H), 3.43 (s, 3H), 1.48 (d, J = 6.3 Hz, 3H); ES-MS [M + H]⁺ = 463 |
| 153 | 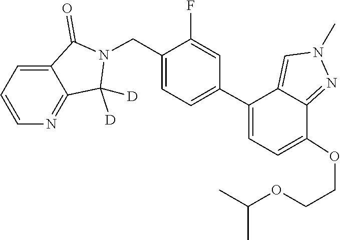 [SMILES: [2H]C1([2H])c2ncccc2C(=O)N1Cc1ccc(-c2ccc(OCCOC(C)C)c3nn(C)cc23)cc1F] | 6-(2-fluoro-4-(7-(2- isopropoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.74-8.68 (m, 1H), 8.15 (dd, J = 7.7, 1.6 Hz, 1H), 7.99 (s, 1H), 7.47-7.39 (m, 2H), 7.39-7.31 (m, 2H), 7.04 (d, J = 7.7 Hz, 1H), 6.68 (d, J = 7.7 Hz, 1H), 4.94 (s, 2H), 4.36 (t, J = 6.0 Hz, 2H), 4.23 (s, 3H), 3.94 (t, J = 6.0 Hz, 2H), 3.71 (septet, J = 6.1 Hz, 1H), 1.20 (d, J = 6.1 Hz, 6H); ES-MS [M + H]⁺ = 477 |
| 154 | 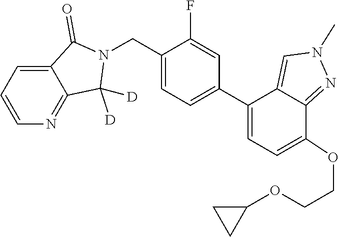 [SMILES: [2H]C1([2H])c2ncccc2C(=O)N1Cc1ccc(-c2ccc(OCCOC3CC3)c3nn(C)cc23)cc1F] | 6-(4-(7-(2- cyclopropoxyethoxy)-2- methyl-2H-indazol-4- yl)-2-fluorobenzyl)-6,7- dihydro-5H-pyrrolo[3,4- b]pyridin-5-one-7,7-d₂ | ¹H NMR (400 MHz, CDCl₃) δ 8.72 (dd, J = 5.0, 1.6 Hz, 1H), 8.16 (dd, J = 7.7, 1.6 Hz, 1H), 7.99 (s, 1H), 7.47-7.40 (m, 2H), 7.40- 7.32 (m, 2H), 7.04 (d, J = 7.7 Hz, 1H), 6.65 (d, J = 7.7 Hz, 1H), 4.94 (s, 2H), 4.37 (t, J = 5.5 Hz, 2H), 4.23 (s, 3H), 4.02 (t, J = 5.5 Hz, 2H), 3.43 (tt, J = 6.0, 3.0 Hz, 1H), 0.66-0.57 (m, 2H), 0.56-0.46 (m, 2H); ES-MS [M + H]⁺ = 475 |
| 155 | 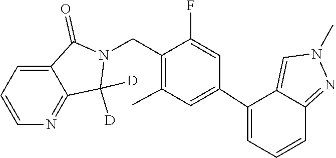 [SMILES: [2H]C1([2H])c2ncccc2C(=O)N1Cc1c(C)cc(-c2cccc3nn(C)cc23)cc1F] | 6-(2-fluoro-6-methyl-4- (2-methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.57 (d, J = 1.0 Hz, 1H), 8.13 (dd, J = 7.7, 1.6 Hz, 1H), 7.61 (dt, J = 8.6, 0.9 Hz, 1H), 7.53 (dd, J = 7.7, 4.9 Hz, 1H), 7.47-7.38 (m, 2H), 7.31 (dd, J = 8.6, 6.9 Hz, 1H), 7.21 (dd, J = 7.0, 0.8 Hz, 1H), 4.89 (d, J = 1.8 Hz, 2H), 4.18 (s, 3H), 2.50 (s, 3H); ES-MS [M + H]⁺ = 389.1 |
| 156 | 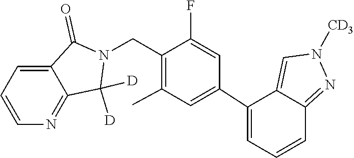 [SMILES: [2H]C1([2H])c2ncccc2C(=O)N1Cc1c(C)cc(-c2cccc3nn(C([2H])([2H])[2H])cc23)cc1F] | 6-(2-fluoro-6-methyl-4- (2-(methyl-d₃)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.57 (d, J = 1.0 Hz, 1H), 8.13 (dd, J = 7.7, 1.6 Hz, 1H), 7.61 (dt, J = 8.6, 0.9 Hz, 1H), 7.53 (dd, J = 7.7, 4.9 Hz, 1H), 7.45 (d, J = 1.8 Hz, 1H), 7.42 (dd, J = 10.9, 1.8 Hz, 1H), 7.32 (dd, J = 8.6, 6.9 Hz, 1H), 7.21 (dd, J = 7.0, 0.8 Hz, 1H), 4.89 (d, J = 1.7 Hz, 2H), 2.50 (s, 3H); ES-MS [M + H]⁺ = 392.1 |
| 157 | 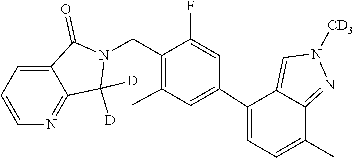 [SMILES: [2H]C1([2H])c2ncccc2C(=O)N1Cc1c(C)cc(-c2ccc(C)c3nn(C([2H])([2H])[2H])cc23)cc1F] | 6-(2-fluoro-6-methyl-4- (7-methyl-2-(methyl-d₃)- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.74 (dd, J = 4.9, 1.6 Hz, 1H), 8.56 (s, 1H), 8.12 (dd, J = 7.7, 1.6 Hz, 1H), 7.53 (dd, J = 7.7, 4.9 Hz, 1H), 7.42 (d, J = 1.8 Hz, 1H), 7.38 (dd, J = 11.1, 1.8 Hz, 1H), 7.16-7.05 (m, 2H), 4.88 (d, J = 1.7 Hz, 2H), 2.53 (s, 3H), 2.49 (s, 3H); ES-MS [M + H]⁺ = 406.2 |
| 158 | 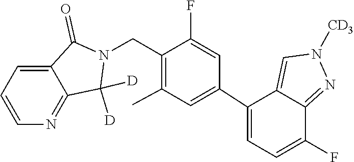 [SMILES: [2H]C1([2H])c2ncccc2C(=O)N1Cc1c(C)cc(-c2ccc(F)c3nn(C([2H])([2H])[2H])cc23)cc1F] | 6-(2-fluoro-4-(7-fluoro- 2-(methyl-d₃)-2H- indazol-4-yl)-6- methylbenzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.70 (d, J = 3.0 Hz, 1H), 8.13 (dd, J = 7.7, 1.6 Hz, 1H), 7.53 (dd, J = 7.7, 4.9 Hz, 1H), 7.44-7.37 (m, 2H), 7.19-7.06 (m, 2H); 2.49 (s, 3H); ES-MS [M + H]⁺ = 410.1 |
| 159 | 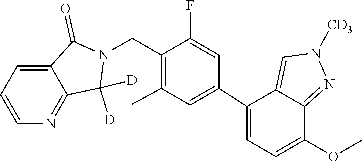 [SMILES: [2H]C1([2H])c2ncccc2C(=O)N1Cc1c(C)cc(-c2ccc(OC)c3nn(C([2H])([2H])[2H])cc23)cc1F] | 6-(2-fluoro-4-(7- methoxy-2-(methyl-d₃)- 2H-indazol-4-yl)-6- methylbenzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.54 (s, 1H), 8.12 (dd, J = 7.7, 1.6 Hz, 1H), 7.53 (dd, J = 7.7, 4.9 Hz, 1H), 7.39 (d, J = 1.8 Hz, 1H), 7.34 (dd, J = 11.2, 1.8 Hz, 1H), 7.14 (d, J = 7.7 Hz, 1H), 6.69 (d, J = 7.8 Hz, 1H), 4.87 (d, J = 1.6 Hz, 2H), 2.48 (s, 3H); ES-MS [M + H]⁺ = 422.1 |
| 160 | 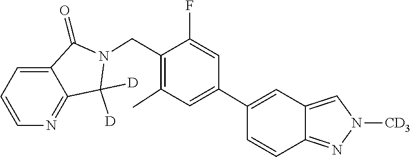 [SMILES: [2H]C1([2H])c2ncccc2C(=O)N1Cc1c(C)cc(-c2ccc3nn(C([2H])([2H])[2H])cc3c2)cc1F] | 6-(2-fluoro-6-methyl-4- (2-(methyl-d₃)-2H- indazol-5-yl)benzyl)-6,7- dihydro-5H-pyrrolo[3,4- b]pyridin-5-one-7,7-d₂ | ¹H NMR (400 MHz, DMSO-d₆) δ 8.73 (dd, J = 4.9, 1.6 Hz, 1H), 8.40 (d, J = 0.9 Hz, 1H), 8.12 (dd, J = 7.7, 1.6 Hz, 1H), 8.04 (dd, J = 1.8, 0.9 Hz, 1H), 7.66 (dt, J = 9.0, 1.0 Hz, 1H), 7.59 (dd, J = 9.1, 1.8 Hz, 1H), 7.52 (dd, J = 7.7, 4.9 Hz, 1H), 7.50-7.41 (m, 2H), 4.85 (d, J = 1.6 Hz, 2H), 2.47 (s, 3H); ES-MS [M + H]⁺ = 392.2 |
| 161 | 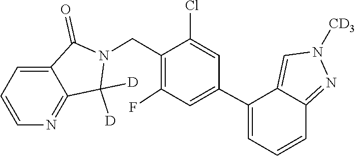 [SMILES: [2H]C1([2H])c2ncccc2C(=O)N1Cc1c(F)cc(-c2cccc3nn(C([2H])([2H])[2H])cc23)cc1Cl] | 6-(2-chloro-6-fluoro-4- (2-(methyl-d₃)-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.61 (d, J = 1.0 Hz, 1H), 8.13 (dd, J = 7.7, 1.6 Hz, 1H), 7.72-7.59 (m, 3H), 7.54 (dd, J = 7.7, 4.9 Hz, 1H), 7.34 (dd, J = 8.5, 6.9 Hz, 1H), 7.27 (dd, J = 7.0, 0.9 Hz, 1H), 4.99 (d, J = 1.8 Hz, 2H); ES-MS [M + H]⁺ = 412.1 |
| 162 | 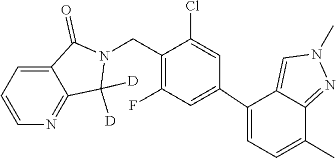 [SMILES: [2H]C1([2H])c2ncccc2C(=O)N1Cc1c(F)cc(-c2ccc(C)c3nn(C)cc23)cc1Cl] | 6-(2-chloro-4-(2,7- dimethyl-2H-indazol-4- yl)-6-fluorobenzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.60 (s, 1H), 8.13 (dd, J = 7.7, 1.6 Hz, 1H), 7.69-7.64 (m, 1H), 7.59 (dd, J = 10.7, 1.7 Hz, 1H), 7.53 (dd, J = 7.7, 4.9 Hz, 1H), 7.20 (d, J = 7.0 Hz, 1H), 7.11 (dd, J = 7.0, 1.2 Hz, 1H), 4.98 (d, J = 1.7 Hz, 2H), 4.20 (s, 3H), 2.54 (s, 3H); ES-MS [M + H]⁺ = 423.1 |
| 163 | 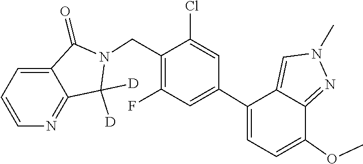 [SMILES: [2H]C1([2H])c2ncccc2C(=O)N1Cc1c(F)cc(-c2ccc(OC)c3nn(C)cc23)cc1Cl] | 6-(2-chloro-6-fluoro-4- (7-methoxy-2-methyl- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.58 (s, 1H), 8.12 (dd, J = 7.7, 1.6 Hz, 1H), 7.63 (t, J = 1.3 Hz, 1H), 7.59-7.50 (m, 2H), 7.22 (d, J = 7.7 Hz, 1H), 6.71 (d, J = 7.8 Hz, 1H), 4.97 (d, J = 1.7 Hz, 2H), 4.16 (s, 3H); 3.94 (s, 3H); ES-MS [M + H]⁺ = 439.0 |
| 164 | 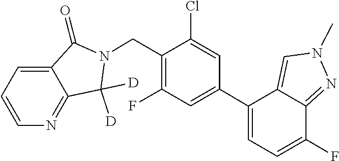 [SMILES: [2H]C1([2H])c2ncccc2C(=O)N1Cc1c(F)cc(-c2ccc(F)c3nn(C)cc23)cc1Cl] | 6-(2-chloro-6-fluoro-4- (7-fluoro-2-methyl-2H- indazol-4-yl)benzyl)-6,7- dihydro-5H-pyrrolo[3,4- b]pyridin-5-one-7,7-d₂ | ¹H NMR (400 MHz, DMSO-d₆) δ 8.78-8.71 (m, 2H), 8.13 (dd, J = 7.7, 1.6 Hz, 1H), 7.70- 7.65 (m, 1H), 7.60 (dd, J = 10.6, 1.7 Hz, 1H), 7.54 (dd, J = 7.7, 4.9 Hz, 1H), 7.24 (dd, J = 7.8, 4.2 Hz, 1H), 7.14 (dd, J = 11.5, 7.8 Hz, 1H), 4.99 (d, J = 1.8 Hz, 2H), 4.22 (s, 3H); ES-MS [M + H]⁺ = 427.0 |
| 165 | 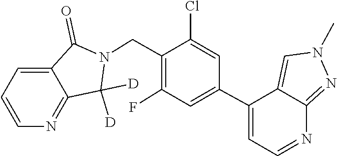 [SMILES: [2H]C1([2H])c2ncccc2C(=O)N1Cc1c(F)cc(-c2ccnc3nn(C)cc23)cc1Cl] | 6-(2-chloro-6-fluoro-4- (2-methyl-2H- pyrazolo[3,4-b]pyridin- 4-yl)benzyl)-6,7- dihydro-5H-pyrrolo[3,4- b]pyridin-5-one-7,7-d₂ | ¹H NMR (400 MHz, DMSO-d₆) δ 8.78-8.72 (m, 2H), 8.67 (d, J = 4.5 Hz, 1H), 8.13 (dd, J = 7.7, 1.6 Hz, 1H), 7.86-7.81 (m, 1H), 7.77 (dd, J = 10.4, 1.7 Hz, 1H), 7.54 (dd, J = 7.7, 4.9 Hz, 1H), 7.37 (d, J = 4.5 Hz, 1H), 5.01 (d, J = 1.8 Hz, 2H), 4.23 (s, 3H); ES-MS [M + H]⁺ = 410.1 |
| 166 | 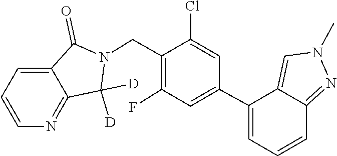 [SMILES: [2H]C1([2H])c2ncccc2C(=O)N1Cc1c(F)cc(-c2cccc3nn(C)cc23)cc1Cl] | 6-(2-chloro-6-fluoro-4- (2-methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.61 (d, J = 1.0 Hz, 1H), 8.13 (dd, J = 7.7, 1.6 Hz, 1H), 7.70 (dd, J = 1.7, 1.7 Hz, 1H), 7.66 (dt, J = 8.5, 0.9 Hz, 1H), 7.62 (dd, J = 10.6, 1.7 Hz, 1H), 7.54 (dd, J = 7.7, 4.9 Hz, 1H), 7.34 (dd, J = 8.5, 7.0 Hz, 1H), 7.27 (dd, J = 7.0, 0.9 Hz, 1H), 4.99 (d, J = 1.8 Hz, 2H), 4.19 (s, 3H); ES-MS [M + H]⁺ = 409.1 |
| 167 | 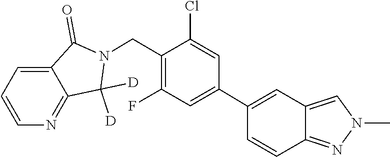 [SMILES: [2H]C1([2H])c2ncccc2C(=O)N1Cc1c(F)cc(-c2ccc3nn(C)cc3c2)cc1Cl] | 6-(2-chloro-6-fluoro-4- (2-methyl-2H-indazol-5- yl)benzyl)-6,7-dihydro- 5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.43 (s, 1H), 8.16- 8.09 (m, 2H), 7.77-7.72 (m, 1H), 7.72- 7.60 (m, 3H), 7.53 (dd, J = 7.7, 5.0 Hz, 1H), 4.96 (d, J = 1.7 Hz, 2H), 4.19 (s, 3H); ES-MS [M + H]⁺ = 409.1 |
| 168 | 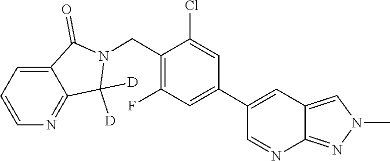 [SMILES: [2H]C1([2H])c2ncccc2C(=O)N1Cc1c(F)cc(-c2cnc3nn(C)cc3c2)cc1Cl] | 6-(2-chloro-6-fluoro-4- (2-methyl-2H- pyrazolo[3,4-b]pyridin- 5-yl)benzyl)-6,7- dihydro-5H-pyrrolo[3,4- b]pyridin-5-one-7,7-d₂ | ¹H NMR (400 MHz, DMSO-d₆) δ 8.97 (d, J = 2.4 Hz, 1H), 8.75 (dd, J = 4.9, 1.6 Hz, 1H), 8.60 (d, J = 2.4 Hz, 1H), 8.53 (s, 1H), 8.12 (dd, J = 7.7, 1.6 Hz, 1H), 7.87-7.82 (m, 1H), 7.77 (dd, J = 11.0, 1.8 Hz, 1H), 7.53 (dd, J = 7.7, 4.9 Hz, 1H), 4.97 (d, J = 1.7 Hz, 2H), 4.23 (s, 3H); ES-MS [M + H]⁺ = 410.1 |
| 169 | 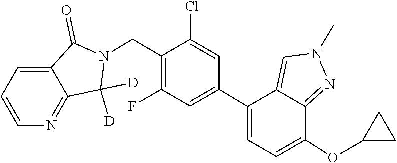 [SMILES: [2H]C1([2H])c2ncccc2C(=O)N1Cc1c(F)cc(-c2ccc(OC3CC3)c3nn(C)cc23)cc1Cl] | 6-(2-chloro-4-(7- cyclopropoxy-2-methyl- 2H-indazol-4-yl)-6- fluorobenzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.58 (s, 1H), 8.13 (dd, J = 7.7, 1.6 Hz, 1H), 7.67-7.60 (m, 1H), 7.58-7.51 (m, 2H), 7.24 (d, J = 7.7 Hz, 1H), 6.99 (d, J = 7.8 Hz, 1H), 4.98 (d, J = 1.7 Hz, 2H), 4.15 (s, 3H), 4.04 (m, 1H), 0.93-0.69 (m, 4H); ES-MS [M + H]⁺ = 465.0 |
| 170 | 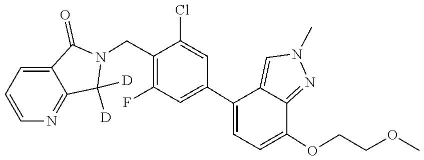 [SMILES: [2H]C1([2H])c2ncccc2C(=O)N1Cc1c(F)cc(-c2ccc(OCCOC)c3nn(C)cc23)cc1Cl] | 6-(2-chloro-6-fluoro-4- (7-(2-methoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.58 (s, 1H), 8.12 (dd, J = 7.7, 1.6 Hz, 1H), 7.63 (t, J = 1.3 Hz, 1H), 7.59-7.49 (m, 2H), 7.20 (d, J = 7.7 Hz, 1H), 6.72 (d, J = 7.9 Hz, 1H), 4.97 (d, J = 1.7 Hz, 2H), 4.33-4.23 (m, 2H), 4.17 (s, 3H), 3.79-3.72 (m, 2H), 3.35 (s, 3H); ES-MS [M + H]⁺ = 483.1 |
| 171 | 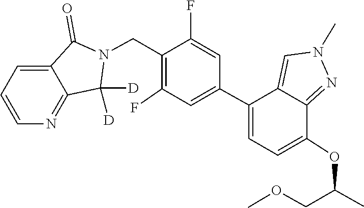 [SMILES: [2H]C1([2H])c2ncccc2C(=O)N1Cc1c(F)cc(-c2ccc(O[C@@H](C)COC)c3nn(C)cc23)cc1F] | (S)-6-(2,6-difluoro-4-(7- ((1-methoxypropan-2- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.01 (s, 1H), 7.40 (dd, J = 7.7, 4.9 Hz, 1H), 7.23-7.16 (m, 2H), 7.06 (d, J = 7.8 Hz, 1H), 6.71 (d, J = 7.8 Hz, 1H), 5.00 (s, 2H), 4.90 (sextet, J = 6.0 Hz, 1H), 4.25 (s, 3H), 3.81 (dd, J = 10.0, 5.8 Hz, 1H), 3.59 (dd, J = 10.0, 5.2 Hz, 1H), 3.43 (s, 3H), 1.48 (d, J = 6.3 Hz, 3H); ES-MS [M + H]⁺ = 481 |
| 172 | 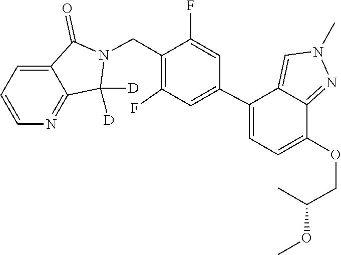 [SMILES: [2H]C1([2H])c2ncccc2C(=O)N1Cc1c(F)cc(-c2ccc(OC[C@@H](C)OC)c3nn(C)cc23)cc1F] | (R)-6-(2,6-difluoro-4-(7- (2-methoxypropoxy)-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.02 (s, 1H), 7.40 (dd, J = 7.7, 5.0 Hz, 1H), 7.25-7.15 (m, 2H), 7.06 (d, J = 7.8 Hz, 1H), 6.66 (d, J = 7.8 Hz, 1H), 5.00 (s, 2H), 4.30 (dd, J = 9.9, 5.9 Hz, 1H), 4.25 (s, 3H), 4.09 (dd, J = 9.9, 5.3 Hz, 1H), 3.95 (sextet, J = 6.1 Hz, 1H), 3.49 (s, 3H), 1.34 (d, J = 6.3 Hz, 3H); ES-MS [M + H]⁺ = 481 |
| 173 | 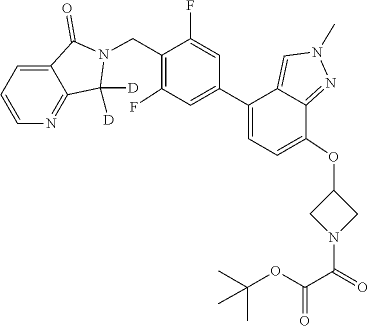 | tert-butyl 3-((4-(3,5- difluoro-4-((5-oxo-5,7- dihydro-6H-pyrrolo[3,4- b]pyridin-6-yl-7,7- d₂)methyl)phenyl)-2- methyl-2H-indaazol-7- yl)oxy)azetidine-1- carboxylate | ¹H NMR (400 MHz, CDCl₃) δ 8.71 (dd, J = 5.0, 1.6 Hz, 1H), 8.15 (dd, J = 7.7, 1.6 Hz, 1H), 8.04 (s, 1H), 7.40 (dd, J = 7.7, 4.9 Hz, 1H), 7.23-7.16 (m, 2H), 7.03 (d, J = 7.6 Hz, 1H), 6.33 (d, J = 7.7 Hz, 1H), 5.15 (tt, J = 6.5, 4.3 Hz, 1H), 5.00 (s, 2H), 4.42-4.35 (m, 2H), 4.29-4.18 (m, 5H), 1.45 (s, 9H); ES- MS [M + H]⁺ = 564 |
| 174 | 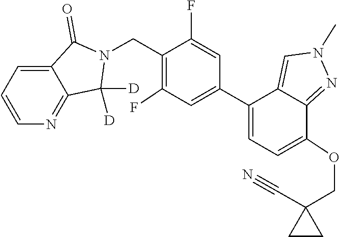 [SMILES: [2H]C1([2H])c2ncccc2C(=O)N1Cc1c(F)cc(-c2ccc(OCC3(C#N)CC3)c3nn(C)cc23)cc1F] | 1-(((4-(3,5-difluoro-4- ((5-oxo-5,7-dihydro-6H- pyrrolo[3,4-b]pyridin-6- yl-7,7- d₂)methyl)phenyl)-2- methyl-2H-indazol-7- yl)oxy)methyl)cyclopro- pane-1-carbonitrile | ¹H NMR (400 MHz, CDCl₃) δ 8.71 (dd, J = 5.0, 1.6 Hz, 1H), 8.15 (dd, J = 7.7, 1.6 Hz, 1H), 8.03 (s, 1H), 7.41 (dd, J = 7.7, 4.9 Hz, 1H), 7.23-7.16 (m, 2H), 7.05 (d, J = 7.7 Hz, 1H), 6.69 (d, J = 7.7 Hz, 1H), 5.00 (s, 2H), 4.34 (s, 2H), 4.26 (s, 3H), 1.47-1.41 (m, 2H), 1.26-1.21 (m, 2H); ES-MS [M + H]⁺ = 488 |
| 175 | 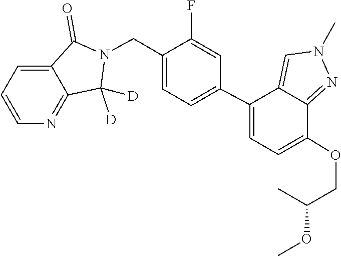 [SMILES: [2H]C1([2H])c2ncccc2C(=O)N1Cc1ccc(-c2ccc(OC[C@@H](C)OC)c3nn(C)cc23)cc1F] | (R)-6-(2-fluoro-4-(7-(2- methoxypropoxy)-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.72 (dd, J = 5.0, 1.6 Hz, 1H), 8.17 (dd, J = 7.7, 1.5 Hz, 1H), 7.99 (s, 1H), 7.47-7.40 (m, 2H), 7.40- 7.32 (m, 2H), 7.05 (d, J = 7.7 Hz, 1H), 6.66 (d, J = 7.8 Hz, 1H), 4.94 (s, 2H), 4.29 (dd, J = 9.9, 5.9 Hz, 1H), 4.24 (s, 3H), 4.09 (dd, J = 9.9, 5.3 Hz, 1H), 3.95 (sextet, J = 6.0 Hz, 1H), 3.49 (s, 3H), 1.34 (d, J = 6.3 Hz, 3H); ES-MS [M + H]⁺ = 463 |
| 176 | 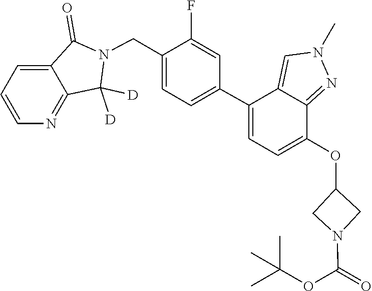 [SMILES: [2H]C1([2H])c2ncccc2C(=O)N1Cc1ccc(-c2ccc(OC3CN(C(=O)OC(C)(C)C)C3)c3nn(C)cc23)cc1F] | tert-butyl 3-((4-(3- fluoro-4-((5-oxo-5,7- dihydro-6H-pyrrolo[3,4- b]pyridin-6-yl-7,7- d₂)methyl)phenyl)-2- methyl-2H-indazol-7- yl)oxy)azetidine-1- carboxylate | ¹H NMR (400 MHz, CDCl₃) δ 8.76-8.69 (m, 1H), 8.17 (dd, J = 7.7, 1.5 Hz, 1H), 8.02 (s, 1H), 7.49-7.40 (m, 2H), 7.39-7.31 (m, 2H), 7.01 (d, J = 7.6 Hz, 1H), 6.32 (d, J = 7.7 Hz, 1H), 5.14 (tt, J = 6.4, 4.3 Hz, 1H), 4.95 (s, 2H), 4.41-4.35 (m, 2H), 4.28-4.20 (m, 5H), 1.45 (s, 9H); ES-MS [M + H]⁺ = 546 |
| 177 | 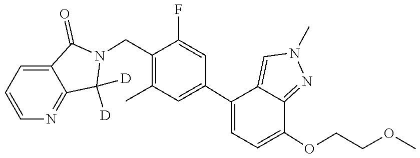 [SMILES: [2H]C1([2H])c2ncccc2C(=O)N1Cc1c(C)cc(-c2ccc(OCCOC)c3nn(C)cc23)cc1F] | 6-(2-fluoro-4-(7-(2- methoxyethoxy)-2- methyl-2H-indazol-4- yl)-6-methylbenzyl)-6,7- dihydro-5H-pyrrolo[3,4- b]pyridin-5-one-7,7-d₂ | 1H NMR (400 MHz, DMSO-d₆) δ 8.74 (dd, J = 4.9, 1.6 Hz, 1H), 8.54 (s, 1H), 8.12 (dd, J = 7.7, 1.6 Hz, 1H), 7.53 (dd, J = 7.7, 4.9 Hz, 1H), 7.39 (d, J = 1.8 Hz, 1H), 7.34 (dd, J = 11.1, 1.8 Hz, 1H), 7.12 (d, J = 7.7 Hz, 1H), 6.70 (d, J = 7.8 Hz, 1H), 4.86 (s, 2H), 4.31-4.27 (m, 2H), 4.17 (s, 3H), 3.78-3.74 (m, 2H), 2.48 (s, 3H); ES-MS [M + H]⁺ = 463.1 |
| 178 | 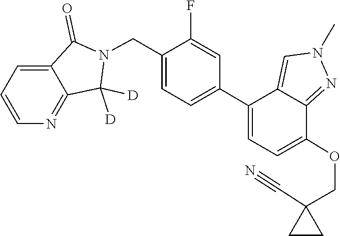 [SMILES: [2H]C1([2H])c2ncccc2C(=O)N1Cc1ccc(-c2ccc(OCC3(C#N)CC3)c3nn(C)cc23)cc1F] | 1-(((4-(3-fluoro-4-((5- oxo-5,7-dihydro-6H- pyrrolo[3,4-b]pyridin-6- yl-7,7- d₂)methyl)phenyl)-2- methyl-2H-indazol-7- yl)oxy)methyl)cyclopro- pane-1-carbonitrile | ¹H NMR (400 MHz, CDCl₃) δ 8.73 (dd, J = 5.0, 1.6 Hz, 1H), 8.19 (dd, J = 7.8, 1.5 Hz, 1H), 8.01 (s, 1H), 7.48-7.41 (m, 2H), 7.40- 7.31 (m, 2H), 7.04 (d, J = 7.6 Hz, 1H), 6.70 (d, J = 7.7 Hz, 1H), 4.95 (s, 2H), 4.34 (s, 2H), 4.25 (s, 3H), 1.47-1.40 (m, 2H), 1.27-1.20 (m, 2H); ES-MS [M + H]⁺ = 470 |
| 179 | 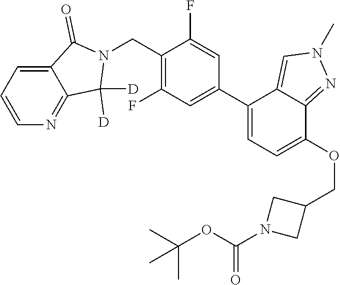 [SMILES: [2H]C1([2H])c2ncccc2C(=O)N1Cc1c(F)cc(-c2ccc(OCC3CN(C(=O)OC(C)(C)C)C3)c3nn(C)cc23)cc1F] | tert-butyl 3-(((4-(3,5- difluoro-4-((5-oxo-5,7- dihydro-6H-pyrrolo[3,4- b]pyridin-6-yl-7,7- d₂)methyl)phenyl)-2- methyl-2H-indazol-7- yl)oxy)methyl)azetidine- 1-carboxylate | ¹H NMR (400 MHz, CDCl₃) δ 8.74-8.68 (m, 1H), 8.16 (dd, J = 7.7, 1.4 Hz, 1H), 8.03 (s, 1H), 7.42 (dd, J = 7.7, 4.9 Hz, 1H), 7.24- 7.17 (m, 2H), 7.07 (d, J = 7.7 Hz, 1H), 6.66 (d, J = 7.8 Hz, 1H), 5.00 (s, 2H), 4.38 (d, J = 7.4 Hz, 2H), 4.25 (s, 3H), 4.17 (t, J = 8.5 Hz, 2H), 3.81 (dd, J = 8.9, 4.9 Hz, 2H), 3.25- 3.12 (m, 1H), 1.44 (s, 9H); ES-MS [M + H]⁺ = 578 |
| 180 | 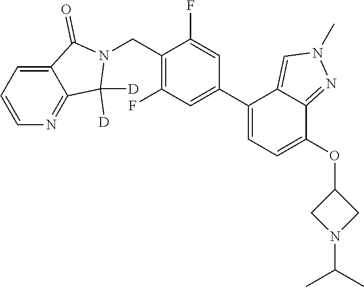 [SMILES: [2H]C1([2H])c2ncccc2C(=O)N1Cc1c(F)cc(-c2ccc(OC3CN(C(C)C)C3)c3nn(C)cc23)cc1F] | 6-(2,6-difluoro-4-(7-((1- isopropylazetidin-3- yl)oxy)-2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J =7.7, 1.6 Hz, 1H), 8.03 (s, 1H), 7.40 (dd, J = 7.7, 4.9 Hz, 1H), 7.23-7.13 (m, 2H), 7.03 (d, J = 7.7 Hz, 1H), 6.45 (d, J = 7.7 Hz, 1H), 5.11 (br s, 1H), 5.00 (s, 2H), 4.26 (s, 3H), 4.15 (br s, 2H), 3.41 (br, 2H), 2.63 (br s, 1H), 1.08 (br s, 6H); ES-MS [M + H]⁺ = 506 |
| 181 | 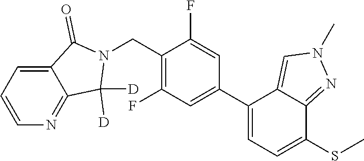 | 6-(2,6-difluoro-4-(2- methyl-7-(methylthio)- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.65 (s, 1H), 8.12 (dd, J = 7.7, 1.6 Hz, 1H), 7.53 (dd, J = 7.7, 4.9 Hz, 1H), 7.49-7.43 (m, 2H), 7.27 (d, J = 7.4 Hz, 1H), 7.07 (d, J = 7.5 Hz, 1H), 4.90 (s, 2H), 4.18 (s, 3H), 2.56 (d, J = 13.1 Hz, 9H); ES-MS [M + H]⁺ = 471.0 |
| 182 | 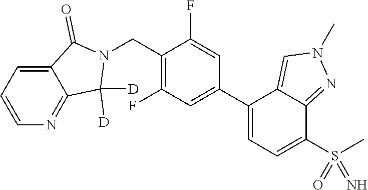 [SMILES: [2H]C1([2H])c2ncccc2C(=O)N1Cc1c(F)cc(-c2ccc(S(C)(=N)=O)c3nn(C)cc23)cc1F] | 6-(2,6-difluoro-4-(2- methyl-7-(S- methylsulfonimidoyl)- 2H-indazol-4-yl)benzyl)- 6,7-dihydro-5H- pyrrolo[3,4-b]pyridin-5- one-7,7-d₂ | ¹H NMR (400 MHz, DMSO-d₆) δ 8.84 (s, 1H), 8.76 (dd, J = 4.9, 1.6 Hz, 1H), 8.12 (dd, J = 7.7, 1.6 Hz, 1H), 7.89 (d, J = 7.4 Hz, 1H), 7.59-7.50 (m, 3H), 7.42 (d, J = 7.4 Hz, 1H), 4.93 (s, 2H), 4.38 (s, 1H), 4.26 (s, 3H), 2.54 (s, 3H); ES-MS [M + H]⁺ = 471.0 |
| 183 | 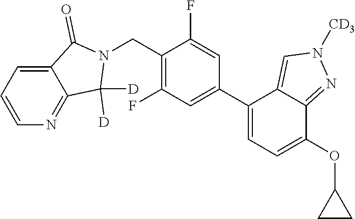 [SMILES: [2H]C1([2H])c2ncccc2C(=O)N1Cc1c(F)cc(-c2ccc(OC3CC3)c3nn(C([2H])([2H])[2H])cc23)cc1F] | 6-(4-(7-cyclopropoxy-2- (methyl-d₃)-2H-indazol- 4-yl)-2,6- difluorobenzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 8.01 (s, 1H), 7.42 (dd, J = 7.7, 5.0 Hz, 1H), 7.24-7.18 (m, 2H), 7.10 (d, J = 7.7 Hz, 1H), 7.01 (d, J = 7.8 Hz, 1H), 5.00 (s, 2H), 3.99-3.92 (m, 1H), 1.03-0.97 (m, 2H), 0.91-0.84 (m, 2H); ES-MS [M + H]⁺ = 452 |
| 184 | 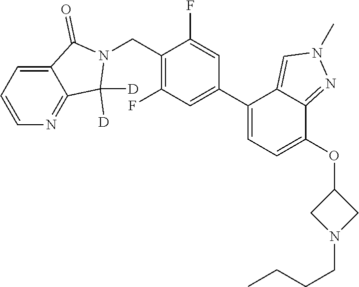 [SMILES: [2H]C1([2H])c2ncccc2C(=O)N1Cc1c(F)cc(-c2ccc(OC3CN(CCCC)C3)c3nn(C)cc23)cc1F] | 6-(4-(7-((1- butylazetidin-3-yl)oxy)- 2-methyl-2H-indazol-4- yl)-2,6-difluorobenzyl)- 6,7-dihydro-5H- pyrrolo[3,4-b]pyridin-5- one-7,7-d₂ | ¹H NMR (400 MHz, CDCl₃) δ 8.71 (dd, J = 4.9, 1.6 Hz, 1H), 8.14 (dd, J = 7.7, 1.6 Hz, 1H), 8.02 (s, 1H), 7.40 (dd, J = 7.7, 5.0 Hz, 1H), 7.23-7.14 (m, 2H), 7.03 (d, J = 7.7 Hz, 1H), 6.42 (d, J = 7.7 Hz, 1H), 5.13-5.04 (m, 1H), 5.00 (s, 2H), 4.26 (s, 3H), 4.10 (br s, 2H), 3.33 (br s, 2H), 2.62 (br s, 2H), 1.46- 1.28 (m, 4H), 0.92 (t, J = 7.1 Hz, 3H); ES- MS [M + H]⁺ = 520 |
| 185 | 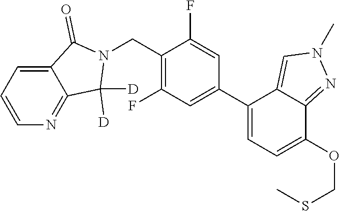 [SMILES: [2H]C1([2H])c2ncccc2C(=O)N1Cc1c(F)cc(-c2ccc(OCSC)c3nn(C)cc23)cc1F] | 6-(2,6-difluoro-4-(2- methyl-7- ((methylthio)methoxy)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.03 (s, 1H), 7.41 (dd, J = 7.7, 5.0 Hz, 1H), 7.24-7.16 (m, 2H), 7.08 (d, J = 7.7 Hz, 1H), 6.75 (d, J = 7.7 Hz, 1H), 5.49 (s, 2H), 5.00 (s, 2H), 4.25 (s, 3H), 2.33 (s, 3H); ES- MS [M + H]⁺ = 469 |
| 186 | 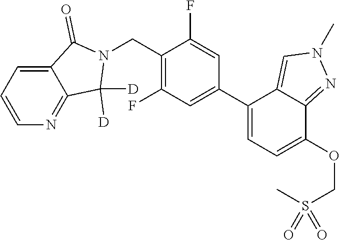 [SMILES: [2H]C1([2H])c2ncccc2C(=O)N1Cc1c(F)cc(-c2ccc(OCS(C)(=O)=O)c3nn(C)cc23)cc1F] | 6-(2,6-difluoro-4-(2- methyl-7-((methyl- sulfonyl)methoxy)- 2H-indazol-4- yl)benzyl)-6,7-dihydro- 5H-pyrrolo[3,4- b]pyridin-5-one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.06 (s, 1H), 7.41 (dd, J = 7.7, 5.0 Hz, 1H), 7.22-7.15 (m, 2H), 7.07 (d, J = 7.7 Hz, 1H, appears as quartet), 7.03 (d, J = 7.8 Hz, 1H, appears as quartet), 5.49 (s, 2H), 5.00 (s, 2H), 4.25 (s, 3H), 3.09 (s, 3H); ES-MS [M + H]⁺ = 501 |
| 187 | 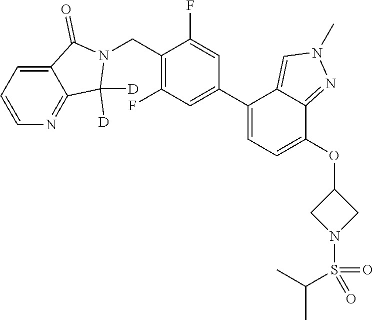 [SMILES: [2H]C1([2H])c2ncccc2C(=O)N1Cc1c(F)cc(-c2ccc(OC3CN(S(=O)(=O)C(C)C)C3)c3nn(C)cc23)cc1F] | 6-(2,6-difluoro-4-(7-((1- (isopropyl- sulfonyl)azetidin- 3-yl)oxy)-2-methyl- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.71 (dd, J = 5.0, 1.6 Hz, 1H), 8.15 (dd, J = 7.7, 1.6 Hz, 1H), 8.04 (s, 1H), 7.41 (dd, J = 7.7, 5.0 Hz, 1H), 7.23-7.14 (m, 2H), 7.02 (d, J = 7.7 Hz, 1H), 6.34 (d, J = 7.6 Hz, 1H), 5.29-5.20 (m, 1H), 5.00 (s, 2H), 4.47-4.40 (m, 2H), 4.31 (dd, J = 9.3, 4.8 Hz, 2H), 4.27 (s, 3H), 3.14 (septet, J = 6.8 Hz, 1H), 1.36 (d, J = 6.8 Hz, 6H); ES-MS [M + H]⁺ = 570 |
| 188 | 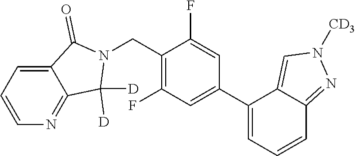 [SMILES: [2H]C1([2H])c2ncccc2C(=O)N1Cc1c(F)cc(-c2cccc3nn(C([2H])([2H])[2H])cc23)cc1F] | 6-(2,6-difluoro-4-(2- (methyl-d₃)-2H- pyrazolo[3,4-b]pyridin- 4-yl)benzyl)-6,7- dihydro-5H-pyrrolo[3,4- b]pyridin-5-one-7,7-d₂ | ¹H NMR (400 MHz, CDCl₃) δ 8.76 (d, J = 4.5 Hz, 1H), 8.72 (dd, J = 5.0, 1.6 Hz, 1H), 8.14 (dd, J = 7.7, 1.6 Hz, 1H), 8.08 (s, 1H), 7.41 (dd, J = 7.7, 4.9 Hz, 1H), 7.36-7.28 (m, 2H), 7.12 (d, J = 4.5 Hz, 1H), 5.02 (s, 2H); ES-MS [M + H]⁺ = 397 |
| 189 | 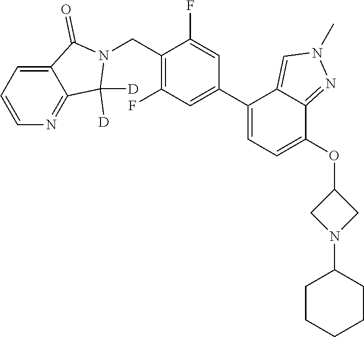 [SMILES: [2H]C1([2H])c2ncccc2C(=O)N1Cc1c(F)cc(-c2ccc(OC3CN(C4CCCCC4)C3)c3nn(C)cc23)cc1F] | 6-(4-(7-((1- cyclohexylazetidin-3- yl)oxy)-2-methyl-2H- indazol-4-yl)-2,6- difluorobenzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 8.02 (s, 1H), 7.40 (dd, J = 7.7, 5.0 Hz, 1H), 7.22-7.15 (m, 2H), 7.03 (d, J = 7.7 Hz, 1H), 6.44 (d, J = 7.7 Hz, 1H), 5.10 (br s, 1H), 5.00 (s, 2H), 4.26 (s, 3H), 4.09 (br s, 2H), 3.36 (br s, 2H), 2.24 (br s, 1H), 1.88-1.70 (m, 4H), 1.32-0.99 (m, 6H); ES-MS [M + H]⁺ = 546 |
| 190 | 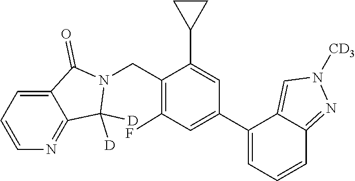 [SMILES: [2H]C1([2H])c2ncccc2C(=O)N1Cc1c(F)cc(-c2cccc3nn(C([2H])([2H])[2H])cc23)cc1C1CC1] | 6-(2-cyclopropyl-6- fluoro-4-(2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.71 (dd, J = 5.0, 1.5 Hz, 1H), 8.13 (dd, J = 7.7, 1.4 Hz, 1H), 7.98 (s, 1H), 7.71 (d, J = 8.6 Hz, 1H), 7.44-7.30 (m, 2H), 7.27-7.24 (m, 1H), 7.15-7.09 (m, 1H), 7.07-7.02 (m, 1H), 5.18 (d, J = 1.9 Hz, 2H), 2.32-2.21 (m, 1H), 1.10-0.99 (m, 2H), 0.75-0.66 (m, 2H); ES-MS [M + H]⁺ = 418.2. |
| 191 | 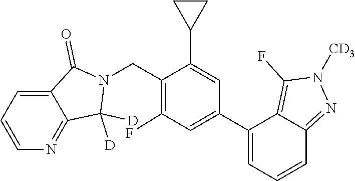 [SMILES: [2H]C1([2H])c2ncccc2C(=O)N1Cc1c(F)cc(-c2cccc3nn(C([2H])([2H])[2H])c(F)c23)cc1C1CC1] | 6-(2-cyclopropyl-6- fluoro-4-(3-fluoro-2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.14 (dd, J = 7.7, 1.6 Hz, 1H), 7.52 (ddd, J = 8.8, 2.1, 0.7 Hz, 1H), 7.41 (dd, J = 7.7, 5.0 Hz, 1H), 7.30 (dd, J = 8.8, 6.9 Hz, 1H), 7.18 (dt, J = 10.7, 2.0 Hz, 1H), 7.03 (d, J = 6.9 Hz, 1H), 6.97 (t, J = 2.4 Hz, 1H), 5.18 (d, J = 1.9 Hz, 2H), 2.25 (ddd, J = 13.5, 8.4, 5.2 Hz, 1H), 1.08-0.98 (m, 2H), 0.76-0.67 (m, 2H); ES-MS [M + H]⁺ = 436.2. |
| 192 | 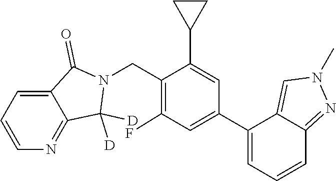 [SMILES: [2H]C1([2H])c2ncccc2C(=O)N1Cc1c(F)cc(-c2cccc3nn(C)cc23)cc1C1CC1] | 6-(2-cyclopropyl-6- fluoro-4-(2-methyl-2H- indazol-4-yl)benzyl)- 6,7-dihydro-5H- pyrrolo[3,4- b]pyridin-5-one-7,7-d₂ | ¹H NMR (400 MHz, CDCl₃) δ 8.70 (dd, J = 5.0, 1.6 Hz, 1H), 8.13 (dd, J = 7.7, 1.6 Hz, 1H), 7.98 (d, J = 1.0 Hz, 1H), 7.70 (dt, J = 8.7, 0.9 Hz, 1H), 7.44-7.29 (m, 2H), 7.26 (dd, J = 10.6, 1.7 Hz, 1H), 7.12 (dd, J = 6.9, 0.8 Hz, 1H), 7.05 (d, J = 1.6 Hz, 1H), 5.17 (d, J = 1.9 Hz, 2H), 4.25 (s, 3H), 2.26 (tt, J = 8.4, 5.3 Hz, 1H), 1.11-0.99 (m, 2H), 0.74-0.66 (m, 2H); ES-MS [M + H]⁺ = 415.1. |
| 193 | 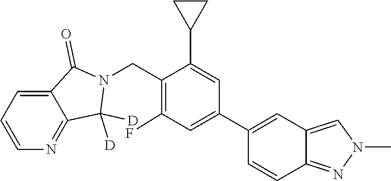 [SMILES: [2H]C1([2H])c2ncccc2C(=O)N1Cc1c(F)cc(-c2ccc3nn(C)cc3c2)cc1C1CC1] | 6-(2-cyclopropyl-6- fluoro-4-(2-methyl-2H- indazol-5-yl)benzyl)- 6,7-dihydro- 5H-pyrrolo[3,4- b]pyridin-5-one-7,7-d₂ | ¹H NMR (400 MHz, CDCl₃) δ 8.70 (dd, J = 5.0, 1.6 Hz, 1H), 8.12 (dd, J = 7.7, 1.6 Hz, 1H), 7.96 (d, J = 0.9 Hz, 1H), 7.81-7.72 (m, 2H), 7.48 (dd, J = 9.0, 1.7 Hz, 1H), 7.39 (dd, J = 7.7, 5.0 Hz, 1H), 7.22 (dd, J = 10.9, 1.8 Hz, 1H), 7.02 (d, J = 1.8 Hz, 1H), 5.15 (d, J = 1.9 Hz, 2H), 4.25 (s, 3H), 2.28- 2.17 (m, 1H), 1.10-0.97 (m, 2H), 0.77- 0.68 (m, 2H); ES-MS [M + H]⁺ = 415.2. |
| 194 | 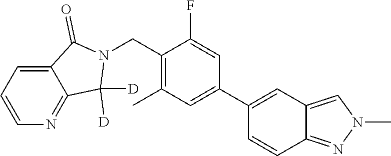 [SMILES: [2H]C1([2H])c2ncccc2C(=O)N1Cc1c(C)cc(-c2ccc3nn(C)cc3c2)cc1F] | 6-(2-fluoro-6-methyl-4- (2-methyl-2H-indazol-5- yl)benzyl)-6,7-dihydro- 5H-pyrrolo[3,4- b]pyridin-5-one-7,7-d₂ | ¹H NMR (400 MHz, DMSO-d₆) δ 8.73 (dd, J = 5.0, 1.6 Hz, 1H), 8.40 (s, 1H), 8.12 (dd, J = 7.7, 1.6 Hz, 1H), 8.06-8.01 (m, 1H), 7.66 (d, J = 9.0 Hz, 1H), 7.59 (dd, J = 9.0, 1.8 Hz, 1H), 7.52 (dd, J = 7.7, 4.9 Hz, 1H), 7.46 (s, 1H), 7.43 (d, J = 11.2 Hz, 1H), 4.85 (d, J = 1.7 Hz, 2H), 4.18 (s, 3H), 2.47 (s, 3H); ES- MS [M + H]⁺ = 389.0. |
| 195 | 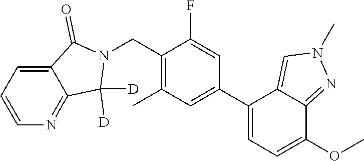 [SMILES: [2H]C1([2H])c2ncccc2C(=O)N1Cc1c(C)cc(-c2ccc(OC)c3nn(C)cc23)cc1F] | 6-(2-fluoro-4-(7- methoxy-2-methyl-2H- indazol-4-yl)-6- methylbenzyl)-6,7- dihydro-5H-pyrrolo[3,4- b]pyridin-5-one-7,7-d₂ | ES-MS [M + H]⁺ = 419.0. |
| 196 | 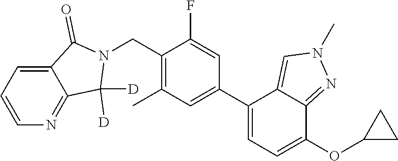 [SMILES: [2H]C1([2H])c2ncccc2C(=O)N1Cc1c(C)cc(-c2ccc(OC3CC3)c3nn(C)cc23)cc1F] | 6-(4-(7-cyclopropoxy-2- methyl-2H-indazol-4- yl)-2-fluoro-6- methylbenzyl)-6,7- dihydro-5H-pyrrolo[3,4- b]pyridin-5-one-7,7-d₂ | ES-MS [M + H]⁺ = 445. |
| 197 | 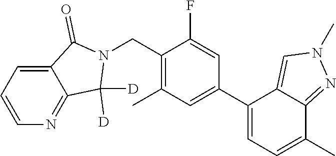 [SMILES: [2H]C1([2H])c2ncccc2C(=O)N1Cc1c(C)cc(-c2ccc(C)c3nn(C)cc23)cc1F] | 6-(4-(2,7-dimethyl-2H- indazol-4-yl)-2-fluoro-6- methylbenzyl)-6,7- dihydro-5H-pyrrolo[3,4- b]pyridin-5-one-7,7-d₂ | ES-MS [M + H]⁺ = 403.0. |
| 198 | 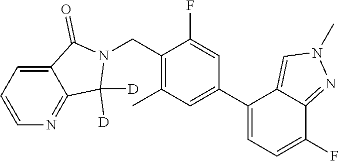 [SMILES: [2H]C1([2H])c2ncccc2C(=O)N1Cc1c(C)cc(-c2ccc(F)c3nn(C)cc23)cc1F] | 6-(2-fluoro-4-(7-fluoro- 2-methyl-2H-indazol-4- yl)-6-methylbenzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.71 (d, J = 3.0 Hz, 1H), 8.13 (dd, J = 7.7, 1.6 Hz, 1H), 7.53 (dd, J = 7.7, 4.9 Hz, 1H), 7.42 (s, 1H), 7.39 (d, J = 10.8 Hz, 1H), 7.19-7.07 (m, 2H), 4.88 (d, J = 1.7 Hz, 2H), 4.21 (s, 3H).; ES-MS [M + H]⁺ = 407.0. |
| 199 | 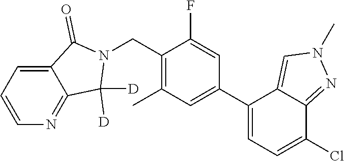 [SMILES: [2H]C1([2H])c2ncccc2C(=O)N1Cc1c(C)cc(-c2ccc(Cl)c3nn(C)cc23)cc1F] | 6-(4-(7-chloro-2-methyl- 2H-indazol-4-yl)-2- fluoro-6-methylbenzyl)- 6,7-dihydro-5H- pyrrolo[3,4-b]pyridin-5- one-7,7-d₂ | ES-MS [M + H]⁺ = 423.0. |
| 200 | 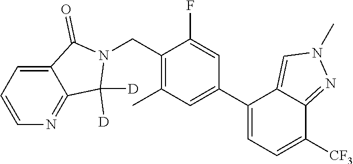 [SMILES: [2H]C1([2H])c2ncccc2C(=O)N1Cc1c(C)cc(-c2ccc(C(F)(F)F)c3nn(C)cc23)cc1F] | 6-(2-fluoro-6-methyl-4- (2-methyl-7- (trifluoromethyl)-2H- indazol-4- yl)benzyl)-6,7-di- hydro-5H-pyrrolo[3,4- b]pyridin-5-one-7,7-d₂ | ¹H NMR (400 MHz, DMSO-d₆) δ 8.79 (s, 1H), 8.75 (dd, J = 5.0, 1.6 Hz, 1H), 8.13 (dd, J = 7.7, 1.6 Hz, 1H), 7.73 (dd, J = 7.4, 1.0 Hz, 1H), 7.53 (dd, J = 7.7, 4.9 Hz, 1H), 7.49 (s, 2H), 7.47 (s, 1H), 7.32 (d, J = 7.3 Hz, 1H), 4.90 (d, J = 1.7 Hz, 2H), 4.25 (s, 3H), 2.52 (s, 3H).; ES-MS [M + H]⁺ = 457.0. |
| 201 | 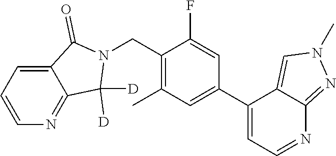 [SMILES: [2H]C1([2H])c2ncccc2C(=O)N1Cc1c(C)cc(-c2ccnc3nn(C)cc23)cc1F] | 6-(2-fluoro-6-methyl-4- (2-methyl-2H- pyrazolo[3,4-b]pyridin- 4-yl)benzyl)-6,7- dihydro-5H-pyrrolo[3,4- b]pyridin-5-one-7,7-d₂ | ES-MS [M + H]⁺ = 390.0. |
| 202 | 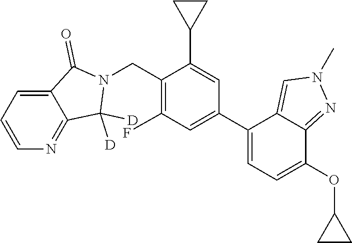 [SMILES: [2H]C1([2H])c2ncccc2C(=O)N1Cc1c(F)cc(-c2ccc(OC3CC3)c3nn(C)cc23)cc1C1CC1] | 6-(4-(7-cyclopropoxy-2- methyl-2H-indazol-4- yl)-2-cyclopropyl-6- fluorobenzyl)-6,7- dihydro-5H-pyrrolo[3,4- b]pyridin-5-one-7,7-d₂ | 1H NMR (400 MHz, CDCl₃) δ 8.70 (dd, J = 5.0, 1.6 Hz, 1H), 8.13 (dd, J = 7.7, 1.6 Hz, 1H), 7.95 (s, 1H), 7.40 (dd, J = 7.7, 4.9 Hz, 1H), 7.22 (dd, J = 10.7, 1.7 Hz, 1H), 7.06 (d, J = 7.7 Hz, 1H), 7.00 (d, J = 7.7 Hz, 2H), 5.16 (d, J = 1.9 Hz, 2H), 4.23 (s, 3H), 3.95 (tt, J = 6.0, 3.0 Hz, 1H), 2.24 (tt, J = 8.4, 5.3 Hz, 1H), 1.10-0.95 (m, 4H), 0.95- 0.81 (m, 2H), 0.74-0.60 (m, 2H); ES-MS [M + H]⁺ = 471.1. |
| 203 | 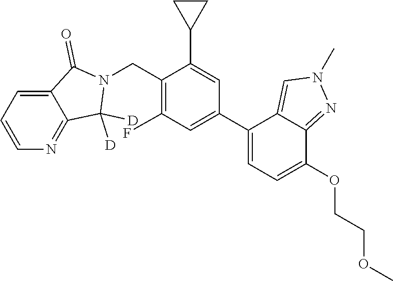 [SMILES: [2H]C1([2H])c2ncccc2C(=O)N1Cc1c(F)cc(-c2ccc(OCCOC)c3nn(C)cc23)cc1C1CC1] | 6-(2-cyclopropyl-6- fluoro-4-(7-(2- methoxyethoxy)-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.69 (dd, J = 5.0, 1.6 Hz, 1H), 8.13 (dd, J = 7.7, 1.6 Hz, 1H), 7.95 (s, 1H), 7.40 (dd, J = 7.7, 5.0 Hz, 1H), 7.20 (dd, J = 10.7, 1.7 Hz, 1H), 7.05- 6.97 (m, 2H), 6.65 (d, J = 7.7 Hz, 1H), 5.15 (d, J = 1.9 Hz, 2H), 4.38 (dd, J = 5.8, 4.6 Hz, 2H), 4.24 (s, 3H), 3.94-3.87 (m, 2H), 3.46 (s, 3H), 2.23 (tt, J = 8.4, 5.3 Hz, 1H), 1.09-0.97 (m, 2H), 0.73-0.65 (m, 2H); ES-MS [M + H]⁺ = 489.1. |
| 204 | 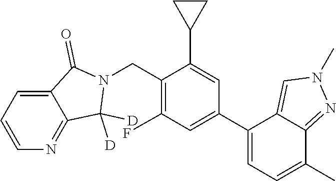 [SMILES: [2H]C1([2H])c2ncccc2C(=O)N1Cc1c(F)cc(-c2ccc(C)c3nn(C)cc23)cc1C1CC1] | 6-(2-cyclopropyl-4-(2,7- dimethyl-2H-indazol-4- yl)-6-fluorobenzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.12 (dd, J = 7.7, 1.6 Hz, 1H), 7.98 (s, 1H), 7.39 (dd, J = 7.7, 5.0 Hz, 1H), 7.24 (dd, J = 10.6, 1.7 Hz, 1H), 7.11 (dd, J = 7.0, 1.2 Hz, 1H), 7.07-7.00 (m, 2H), 5.16 (d, J = 1.9 Hz, 2H), 4.26 (s, 3H), 2.65 (d, J = 1.1 Hz, 3H), 2.25 (tt, J = 8.3, 5.3 Hz, 1H), 1.10-0.98 (m, 2H), 0.74- 0.65 (m, 2H); ES-MS [M + H]⁺ = 429.2. |
| 205 | 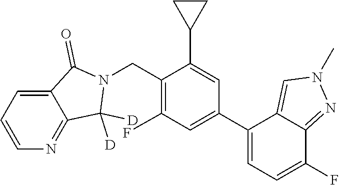 [SMILES: [2H]C1([2H])c2ncccc2C(=O)N1Cc1c(F)cc(-c2ccc(F)c3nn(C)cc23)cc1C1CC1] | 6-(2-cyclopropyl-6- fluoro-4-(7-fluoro-2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.70 (dd, J = 5.0, 1.6 Hz, 1H), 8.13 (dd, J = 7.7, 1.6 Hz, 1H), 8.01 (d, J = 2.7 Hz, 1H), 7.40 (dd, J = 7.7, 4.9 Hz, 1H), 7.20 (dd, J = 10.5, 1.7 Hz, 1H), 7.05-6.97 (m, 3H), 5.16 (d, J = 1.9 Hz, 2H), 4.27 (s, 3H), 2.26 (tt, J = 8.4, 5.3 Hz, 1H), 1.11-0.99 (m, 2H), 0.73-0.65 (m, 2H); ES-MS [M + H]⁺ = 433.1. |
| 206 | 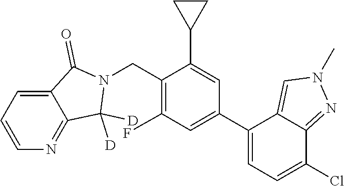 [SMILES: [2H]C1([2H])c2ncccc2C(=O)N1Cc1c(F)cc(-c2ccc(Cl)c3nn(C)cc23)cc1C1CC1] | 6-(4-(7-chloro-2- methyl-2H-indazol-4- yl)-2-cyclopropyl-6- fluorobenzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.13 (dd, J = 7.7, 1.6 Hz, 1H), 8.03 (s, 1H), 7.45-7.32 (m, 2H), 7.22 (dd, J = 10.5, 1.7 Hz, 1H), 7.07-6.98 (m, 2H), 5.17 (d, J = 19 Hz, 2H), 4.29 (s, 3H), 2.26 (tt, J = 8.4, 5.3 Hz, 1H), 1.12-0.99 (m, 2H), 0.74-0.65 (m, 2H)); ES-MS [M + H]⁺ = 449.1 |
| 207 | 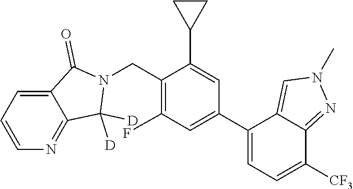 [SMILES: [2H]C1([2H])c2ncccc2C(=O)N1Cc1c(F)cc(-c2ccc(C(F)(F)F)c3nn(C)cc23)cc1C1CC1] | 6-(2-cyclopropyl-6- fluoro-4-(2-methyl-7- (trifluoromethyl)-2H- indazol-4- yl)benzyl)-6,7-di- hydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.13 (dd, J = 7.7, 1.5 Hz, 1H), 8.06 (s, 1H), 7.66 (dd, J = 7.3, 1.1 Hz, 1H), 7.41 (dd, J = 7.7, 4.9 Hz, 1H), 7.28- 7.21 (m, 1H), 7.13 (dd, J = 7.2, 0.9 Hz, 1H), 7.03 (d, J = 1.6 Hz, 1H), 5.18 (d, J = 1.9 Hz, 2H), 4.31 (s, 3H), 2.28 (tt, J = 8.4, 5.3 Hz, 1H), 1.13-1.01 (m, 2H), 0.74-0.66 (m, 2H)); ES-MS [M + H]⁺ = 483.1. |
| 208 | 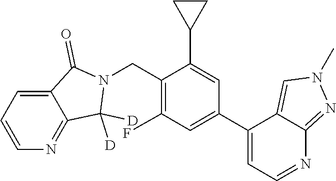 [SMILES: [2H]C1([2H])c2ncccc2C(=O)N1Cc1c(F)cc(-c2ccnc3nn(C)cc23)cc1C1CC1] | 6-(2-cyclopropyl-6- fluoro-4-(2-methyl-2H- pyrazolo[3,4-b]pyridin- 4-yl)benzyl)-6,7- dihydro-5H-pyrrolo[3,4- b]pyridin-5-one-7,7-d₂ | ¹H NMR (400 MHz, CDCl₃) δ 8.75-8.66 (m, 2H), 8.12 (dd, J = 7.7, 1.6 Hz, 1H), 8.02 (s, 1H), 7.40 (dd, J = 7.7, 4.9 Hz, 1H), 7.30 (dd, J = 10.2, 1.7 Hz, 1H), 7.12-7.06 (m, 2H), 5.17 (d, J = 1.9 Hz, 2H), 4.28 (s, 3H), 2.33-2.23 (m, 1H), 1.14-1.01 (m, 2H), 0.75-0.66 (m, 2H); ES-MS [M + H]⁺ = 416.2. |
| 209 | 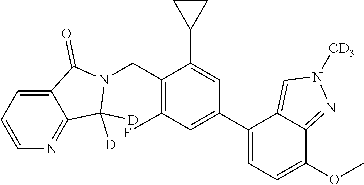 [SMILES: [2H]C1([2H])c2ncccc2C(=O)N1Cc1c(F)cc(-c2ccc(OC)c3nn(C([2H])([2H])[2H])cc23)cc1C1CC1] | 6-(2-cyclopropyl-6- fluoro-4-(7-methoxy-2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.13 (dd, J = 7.7, 1.6 Hz, 1H), 7.96 (s, 1H), 7.40 (dd, J = 7.7, 5.0 Hz, 1H), 7.21 (dd, J = 10.7, 1.7 Hz, 1H), 7.08- 6.98 (m, 2H), 6.62 (d, J = 7.7 Hz, 1H), 5.16 (d, J = 1.9 Hz, 2H), 4.04 (s, 3H), 2.24 (tt, J = 8.4, 5.3 Hz, 1H), 1.10-0.97 (m, 2H), 0.74-0.65 (m, 2H); ES-MS [M + H]⁺ = 448.1. |
| 210 | 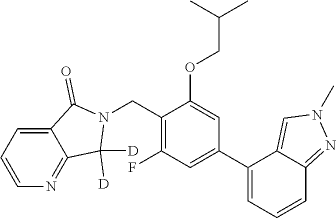 [SMILES: [2H]C1([2H])c2ncccc2C(=O)N1Cc1c(F)cc(-c2cccc3nn(C)cc23)cc1OCC(C)C] | 6-(2-fluoro-6-isobutoxy- 4-(2-methyl-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.73 (dd, J = 4.9, 1.6 Hz, 1H), 8.58 (s, 1H), 8.11 (dd, J = 7.7, 1.6 Hz, 1H), 7.63 (dt, J = 8.5, 0.9 Hz, 1H), 7.52 (dd, J = 7.7, 4.9 Hz, 1H), 7.32 (dd, J = 8.6, 7.0 Hz, 1H), 7.24 (dd, J = 6.9, 0.9 Hz, 1H), 7.19-7.11 (m, 2H), 4.88 (d, J = 1.4 Hz, 2H), 4.18 (s, 3H), 3.95 (d, J = 6.2 Hz, 2H), 2.05 (dq, J = 13.0, 6.6 Hz, 1H), 1.00 (d, J = 6.7 Hz, 6H).; ES-MS [M + H]⁺ = 447.0. |
| 211 | 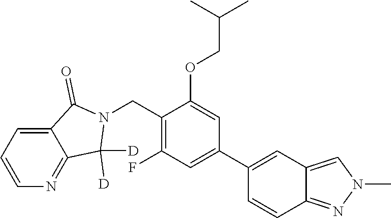 [SMILES: [2H]C1([2H])c2ncccc2C(=O)N1Cc1c(F)cc(-c2ccc3nn(C)cc3c2)cc1OCC(C)C] | 6-(2-fluoro-6-isobutoxy- 4-(2-methyl-2H-indazol- 5-yl)benzyl)-6,7- dihydro-5H-pyrrolo[3,4- b]pyridin-5-one-7,7-d₂ | ¹H NMR (400 MHz, DMSO-d₆) δ 8.72 (dd, J = 4.9, 1.6 Hz, 1H), 8.41 (s, 1H), 8.10 (dd, J = 7.7, 1.6 Hz, 1H), 8.07 (dd, J = 1.8, 1.0 Hz, 1H), 7.67 (dt, J = 9.1, 1.0 Hz, 1H), 7.61 (dd, J = 9.0, 1.7 Hz, 1H), 7.51 (dd, J = 7.7, 5.0 Hz, 1H), 7.21- 7.13 (m, 2H), 4.84 (d, J = 1.4 Hz, 2H), 4.19 (s, 3H), 3.96 (d, J = 6.3 Hz, 2H), 2.05 (hept, J = 6.6 Hz, 1H), 1.00 (d, J = 6.7 Hz, 6H).; ES-MS [M + H]⁺ = 447.0. |
| 212 | 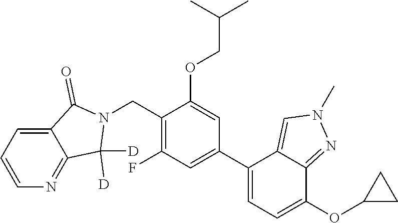 [SMILES: [2H]C1([2H])c2ncccc2C(=O)N1Cc1c(F)cc(-c2ccc(OC3CC3)c3nn(C)cc23)cc1OCC(C)C] | 6-(4-(7-cyclopropoxy-2- methyl-2H-indazol-4- yl)-2-fluoro-6- isobutoxybenzyl)-6,7- dihydro-5H-pyrrolo[3,4- b]pyridin-5-one-7,7-d₂ | ¹H NMR (400 MHz, DMSO-d₆) δ 8.72 (dd, J = 4.9, 1.6 Hz, 1H), 8.54 (s, 1H), 8.10 (dd, J = 7.7, 1.6 Hz, 1H), 7.51 (dd, J = 7.7, 4.9 1Hz, H), 7.19 (d, J = 7.7 Hz, 1H), 7.12-7.04 (m, 2H), 6.97 (d, J = 7.7 Hz, 1H), 4.86 (d, J = 1.3 Hz, 2H), 4.15 (s, 3H), 4.04 (tt, J = 6.1, 3.0 Hz, 1H), 3.94 (d, J = 6.2 Hz, 2H), 2.11-1.99 (m, 1H), 1.00 (d, J = 6.7 Hz, 6H), 0.90-0.80 (m, 2H), 0.80-0.70 (m, 2H).; ES-MS [M + H]⁺ = 503.0. |
| 213 | 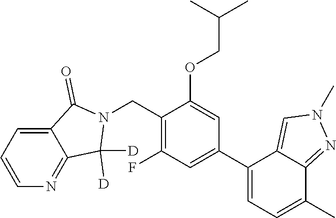 [SMILES: [2H]C1([2H])c2ncccc2C(=O)N1Cc1c(F)cc(-c2ccc(C)c3nn(C)cc23)cc1OCC(C)C] | 6-(4-(2,7-dimethyl-2H- indazol-4-yl)-2-fluoro-6- isobutoxybenzyl)-6,7- dihydro-5H-pyrrolo[3,4- b]pyridin-5-one-7,7-d₂ | ¹H NMR (400 MHz, DMSO-d₆) δ 8.73 (dd, J = 4.9, 1.6 Hz, 1H), 8.56 (s, 1H), 8.11 (dd, J = 7.7, 1.6 Hz, 1H), 7.52 (dd, J = 7.7, 4.9 Hz, 1H), 7.16 (d, J = 7.0 Hz, 1H), 7.14-7.05 (m, 3H), 4.87 (s, 2H), 4.19 (s, 3H), 3.94 (d, J = 6.2 Hz, 2H), 2.53 (d, J = 1.0 Hz, 3H), 2.13-1.98 (m, 1H), 1.00 (d, J = 6.7 Hz, 6H).; ES-MS [M + H]⁺ = 461.0. |
| 214 | 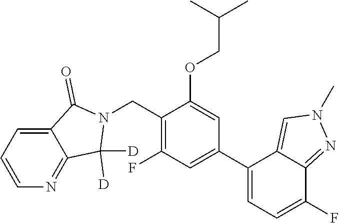 [SMILES: [2H]C1([2H])c2ncccc2C(=O)N1Cc1c(F)cc(-c2ccc(F)c3nn(C)cc23)cc1OCC(C)C] | 6-(2-fluoro-4-(7-fluoro- 2-methyl-2H-indazol-4- yl)-6-isobutoxybenzyl)- 6,7-dihydro-5H- pyrrolo[3,4-b]pyridin-5- one-7,7-d₂ | ¹H NMR (400 MHz, DMSO-d₆) δ 8.76-8.69 (m, 2H), 8.11 (dd, J = 7.7, 1.6 Hz, 1H), 7.52 (dd, J = 7.7, 4.9 Hz, 1H), 7.20 (dd, J = 7.8, 4.2 Hz, 1H), 7.17-7.07 (m, 3H), 4.87 (d, J = 1.3 Hz, 2H), 4.22 (s, 3H), 3.94 (d, J = 6.2 Hz, 2H), 2.11-1.98 (m, 1H), 1.00 (d, J = 6.7 Hz, 6H).; ES-MS [M + H]⁺ = 465.0. |
| 215 | 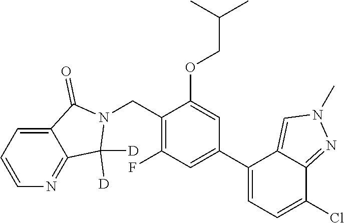 [SMILES: [2H]C1([2H])c2ncccc2C(=O)N1Cc1c(F)cc(-c2ccc(Cl)c3nn(C)cc23)cc1OCC(C)C] | 6-(4-(7-chloro-2-methyl- 2H-indazol-4-yl)-2- fluoro-6- isobutoxybenzyl)-6,7- dihydro-5H-pyrrolo[3,4- b]pyridin-5-one-7,7-d₂ | ¹H NMR (400 MHz, DMSO-d₆) δ 8.76-8.70 (m, 2H), 8.11 (dd, J = 7.7, 1.6 Hz, 1H), 7.52 (dd, J = 7.7, 4.9 Hz, 1H), 7.44 (d, J = 7.5 Hz, 1H), 7.23 (d, J = 7.5 Hz, 1H), 7.19-7.10 (m, 2H), 4.90-4.85 (m, 2H), 4.22 (s, 3H), 3.95 (d, J = 6.2 Hz, 2H), 2.11-2.00 (m, 1H), 1.00 (d, J = 6.7 Hz, 6H).; ES-MS [M + H]⁺ = 481.0. |
| 216 | 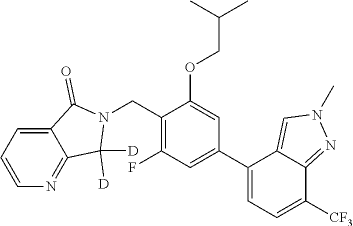 [SMILES: [2H]C1([2H])c2ncccc2C(=O)N1Cc1c(F)cc(-c2ccc(C(F)(F)F)c3nn(C)cc23)cc1OCC(C)C] | 6-(2-fluoro-6-isobutoxy- 4-(2-methyl-7- (trifluoromethyl)-2H- indazol-4-yl) benzyl)-6,7-di- hydro-5H-pyrrolo[3,4- b]pyridin-5-one-7,7-d₂ | ¹H NMR (400 MHz, DMSO-d₆) δ 8.80 (s, 1H), 8.73 (dd, J = 4.9, 1.6 Hz, 1H), 8.11 (dd, J = 7.7, 1.6 Hz, 1H), 7.73 (d, J = 7.0 Hz, 1H), 7.52 (dd, J = 7.7, 4.9 Hz, 1H), 7.36 (d, J = 7.3 Hz, 1H), 7.21 (dd, J = 10.1, 1.4 Hz, 1H), 7.18 (s, 1H), 4.89 (d, J = 1.4 Hz, 2H), 4.25 (s, 3H), 3.96 (d, J = 6.2 Hz, 2H), 2.13-1.99 (m, 1H), 1.00 (d, J = 6.7 Hz, 6H).; ES-MS [M + H]⁺ = 515.0. |
| 217 | 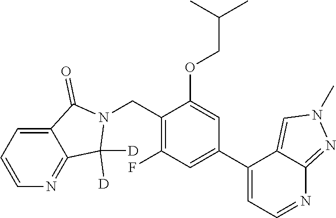 [SMILES: [2H]C1([2H])c2ncccc2C(=O)N1Cc1c(F)cc(-c2ccnc3nn(C)cc23)cc1OCC(C)C] | 6-(2-fluoro-6-isobutoxy- 4-(2-methyl-2H- pyrazolo[3,4-b]pyridin- 4-yl)benzyl)-6,7- dihydro-5H-pyrrolo[3,4- b]pyridin-5-one-7,7-d₂ | ¹H NMR (400 MHz, DMSO-d₆) δ 8.76-8.70 (m, 2H), 8.65 (d, J = 4.5 Hz, 1H), 8.11 (dd, J = 7.7, 1.6 Hz, 1H), 7.52 (dd, J = 7.7, 5.0 Hz, 1H), 7.34 (d, J = 4.5 Hz, 1H), 7.29 (dd, J = 10.1, 1.4 Hz, 1H), 7.24 (d, J = 1.5 Hz, 1H), 4.89 (d, J = 1.5 Hz, 2H), 4.22 (s, 3H), 3.98 (d, J = 6.2 Hz, 2H), 2.06 (hept, J = 6.5 Hz, 1H), 1.01 (d, J = 6.7 Hz, 6H).; ES-MS [M + H]⁺ = 448.0. |
| 218 | 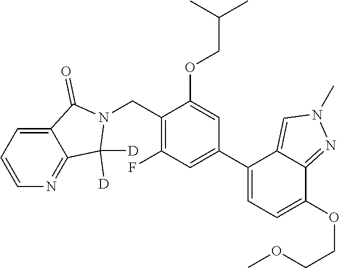 [SMILES: [2H]C1([2H])c2ncccc2C(=O)N1Cc1c(F)cc(-c2ccc(OCCOC)c3nn(C)cc23)cc1OCC(C)C] | 6-(2-fluoro-6-isobutoxy- 4-(7-(2-methoxyethoxy)- 2-methyl-2H-indazol-4- yl)benzyl)-6,7-dihydro- 5H-pyrrolo[3,4- b]pyridin-5-one-7,7-d₂ | ES-MS [M + H]⁺ = 521.0. |
| 219 | 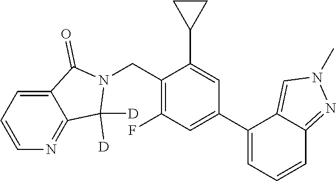 | 6-(2-chloro-6- cyclopropyl-4-(2- methyl-2H-indazol-4- yl)benzyl)-6,7-dihydro- 5H-pyrrolo[3,4-]pyridin- 5-one-7,7-d₂ | ¹H NMR (400 MHz, CDCl₃) δ 8.70 (dd, J = 5.0, 1.6 Hz, 1H), 8.14 (dd, J = 7.7, 1.6 Hz, 1H), 7.98 (s, 1H), 7.71 (d, J = 8.7 Hz, 1H), 7.59 (d, J = 1.7 Hz, 1H), 7.41 (dd, J = 7.7, 5.0 Hz, 1H), 7.35 (dd, J = 8.7, 6.9 Hz, 1H), 7.18 (d, J = 1.7 Hz, 1H), 7.12 (d, J = 6.9 Hz, 1H), 5.33 (s, 2H), 4.26 (s, 3H), 2.18 (tt, J = 8.3, 5.4 Hz, 1H), 1.11-0.98 (m, 2H), 0.73- 0.65 (m, 2H); ES-MS [M + H]⁺ = 431.1. |
| 220 | 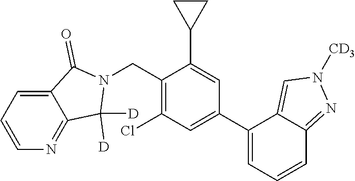 [SMILES: [2H]C1([2H])c2ncccc2C(=O)N1Cc1c(Cl)cc(-c2cccc3nn(C([2H])([2H])[2H])cc23)cc1C1CC1] | 6-(2-chloro-6- cyclopropyl-4-(2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.70 (dd, J = 5.0, 1.6 Hz, 1H), 8.15 (dd, J = 7.7, 1.6 Hz, 1H), 7.99 (d, J = 1.0 Hz, 1H), 7.71 (d, J = 8.7 Hz, 1H), 7.59 (d, J = 1.8 Hz, 1H), 7.42 (dd, J = 7.7, 5.0 Hz, 1H), 7.36 (dd, J = 8.7, 6.9 Hz, 1H), 7.18 (d, J = 1.8 Hz, 1H), 7.12 (d, J = 6.9 Hz, 1H), 5.33 (s, 2H), 2.19 (tt, J = 8.4, 5.4 Hz, 1H), 1.11-0.98 (m, 2H), 0.73-0.65 (m, 2H); ES-MS [M + H]⁺ = 434.1. |
| 221 | 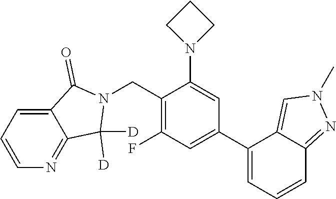 [SMILES: [2H]C1([2H])c2ncccc2C(=O)N1Cc1c(F)cc(-c2cccc3nn(C)cc23)cc1N1CCC1] | 6-(2-(azetidin-1-yl)-6- fluoro-4-(2-methyl-2H- indazol-4-yl)benzyl)-6,7- dihydro-5H-pyrrolo[3,4- b]pyridin-5-one-7,7-d₂ | ¹H NMR (400 MHz, CDCl₃) δ 8.69 (dd, J = 5.0, 1.6 Hz, 1H), 8.13 (dd, J = 7.7, 1.6 Hz, 1H), 8.04 (s, 1H), 7.70 (d, J = 8.6 Hz, 1H), 7.41-7.31 (m, 2H), 7.16 (dd, J = 6.9, 0.8 Hz, 1H), 6.87 (dd, J = 10.8, 1.6 Hz, 1H), 6.52 (s, 1H), 4.93 (d, J = 2.1 Hz, 2H), 4.24 (s, 3H), 4.10 (t, J = 7.3 Hz, 4H), 2.35- 2.24 (m, 2H); ES-MS [M + H]⁺ = 430.1. |
| 222 | 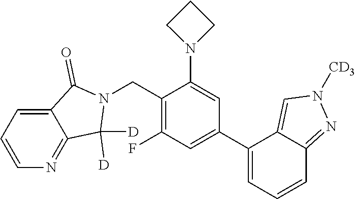 [SMILES: [2H]C1([2H])c2ncccc2C(=O)N1Cc1c(F)cc(-c2cccc3nn(C([2H])([2H])[2H])cc23)cc1N1CCC1] | 6-(2-(azetidin-1-yl)-6- fluoro-4-(2-(methyl-d₃)- 2H-indazol-4-yl)benzyl)- 6,7-dihydro-5H- pyrrolo[3,4-b]pyridin-5- one-7,7-d₂ | ¹H NMR (400 MHz, CDCl₃) δ 8.70 (dd, J = 5.0, 1.6 Hz, 1H), 8.13 (dd, J = 7.7, 1.6 Hz, 1H), 8.04 (d, J = 1.0 Hz, 1H), 7.70 (d, J = 8.7 Hz, 1H), 7.44-7.29 (m, 2H), 7.16 (dd, J = 6.9, 0.8 Hz, 1H), 6.87 (dd, J = 10.8, 1.6 Hz, 1H), 6.53 (s, 1H), 4.93 (d, J = 2.1 Hz, 2H), 4.10 (t, J = 7.3 Hz, 4H), 2.35-2.26 (m, 2H); ES-MS [M + H]⁺ = 433.2. |
| 223 | 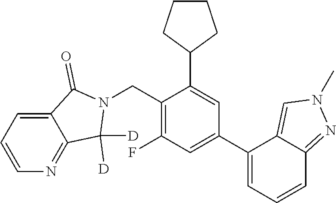 [SMILES: [2H]C1([2H])c2ncccc2C(=O)N1Cc1c(F)cc(-c2cccc3nn(C)cc23)cc1C1CCCC1] | 6-(2-cyclopentyl-6- fluoro-4-(2-methyl-2H- indazol-4-yl)benzyl)- 6,7-dihydro-5H- pyrrolo[3,4- b]pyridin-5-one-7,7-d₂ | ¹H NMR (400 MHz, CDCl₃) δ 8.70 (dd, J = 5.0, 1.6 Hz, 1H), 8.15 (dd, J = 7.7, 1.5 Hz, 1H), 8.00 (s, 1H), 7.72 (d, J = 8.7 Hz, 1H), 7.44-7.34 (m, 3H), 7.26 (dd, J = 10.8, 1.6 Hz, 1H), 7.16 (d, J = 6.9 Hz, 1H), 5.04 (d, J = 2.1 Hz, 2H), 4.26 (s, 3H), 3.62-3.49 (m, 1H), 2.07-1.95 (m, 2H), 1.87-1.67 (m, 4H), 1.66-1.50 (m, 2H); ES-MS [M + H]⁺ = 443.2. |
| 224 | 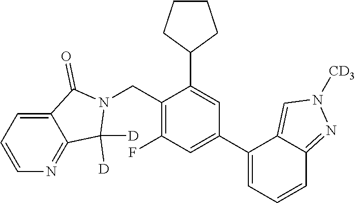 [SMILES: [2H]C1([2H])c2ncccc2C(=O)N1Cc1c(F)cc(-c2cccc3nn(C([2H])([2H])[2H])cc23)cc1C1CCCC1] | 6-(2-cyclopentyl-6- fluoro-4-(2-(methyl-d₃)- 2H-indazol-4- yl)benzyl)- 6,7-dihydro-5H- pyrrolo[3,4-b]pyridin-5- one-7,7-d₂ | 1H NMR (400 MHz, CDCl₃) δ 8.70 (dd, J = 5.0, 1.6 Hz, 1H), 8.15 (dd, J = 7.7, 1.5 Hz, 1H), 8.00 (s, 1H), 7.72 (d, J = 8.7 Hz, 1H), 7.44-7.33 (m, 3H), 7.26 (dd, J = 10.7, 1.6 Hz, 1H), 7.16 (dd, J = 6.9, 0.7 Hz, 1H), 5.04 (d, J = 2.1 Hz, 2H), 3.62-3.49 (m, 1H), 2.07- 1.95 (m, 2H), 1.87-1.66 (m, 4H), 1.66- 1.50 (m, 2H); ES-MS [M + H]⁺ = 446.1. |
| 225 | 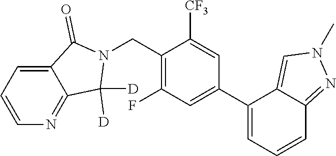 [SMILES: [2H]C1([2H])c2ncccc2C(=O)N1Cc1c(F)cc(-c2cccc3nn(C)cc23)cc1C(F)(F)F] | 6-(2-fluoro-4-(2-methyl- 2H-indazol-4-yl)-6- (trifluoromethyl)benzyl)- 6,7-dihydro-5H- pyrrolo[3,4-b]pyridin-5- one-7,7-d₂ | ¹H NMR (400 MHz, DMSO-d₆) δ: 8.75 (dd, J = 4.9, 1.6 Hz, 1H), 8.62 (s, 1H), 8.15 (dd, J = 7.7, 1.6 Hz, 1H), 7.93 (d, J = 10.8 Hz, 1H), 7.90 (s, 1H), 7.69 (dt, J = 8.2, 1.1 Hz, 1H), 7.54 (dd, J = 7.7, 4.9 Hz, 1H), 7.40-7.30 (m, 2H), 5.04 (s, 2H), 4.20 (s, 3H); ES-MS [M + H]⁺ = 443.0. |
| 226 | 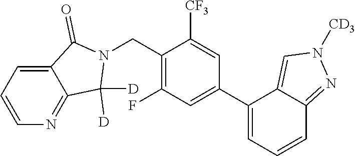 [SMILES: [2H]C1([2H])c2ncccc2C(=O)N1Cc1c(F)cc(-c2cccc3nn(C([2H])([2H])[2H])cc23)cc1C(F)(F)F] | 6-(2-fluoro-4-(2- (methyl-d₃)-2H-indazol- 4-yl)-6- (trifluoromethyl)benzyl)- 6,7-dihydro-5H- pyrrolo[3,4-b]pyridin-5- one-7,7-d₂ | ¹H NMR (400 MHz, DMSO-d₆) δ 8.75 (dd, J = 4.9, 1.6 Hz, 1H), 8.62 (d, J = 1.0 Hz, 1H), 8.15 (dd, J = 7.7, 1.6 Hz, 1H), 7.94 (d, J = 11.1 Hz, 1H), 7.90 (s, 1H), 7.69 (dt, J = 8.2, 1.1 Hz, 1H), 7.54 (dd, J = 7.7, 4.9 Hz, 1H), 7.39-7.29 (m, 2H), 5.04 (s, 2H); ES-MS [M + H]⁺ = 446.0. |
| 227 | 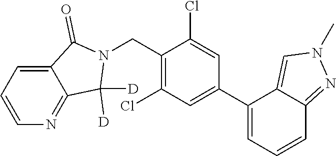 [SMILES: [2H]C1([2H])c2ncccc2C(=O)N1Cc1c(Cl)cc(-c2cccc3nn(C)cc23)cc1Cl] | 6-(2,6-dichloro-4-(2- methyl-2H-indazol-4- yl)benzyl)-6,7-dihydro- 5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.59 (s, 1H), 8.14 (dd, J = 7.7, 1.6 Hz, 1H), 7.84 (s, 2H), 7.67 (d, J = 8.5 Hz, 1H), 7.54 (dd, J = 7.7, 4.9 Hz, 1H), 7.34 (dd, J = 8.5, 6.9 Hz, 1H), 7.28 (dd, J = 7.0, 0.9 Hz, 1H), 5.10 (s, 2H), 4.20 (s, 3H); ES-MS [M + H]⁺ = 425.0. |
| 228 | 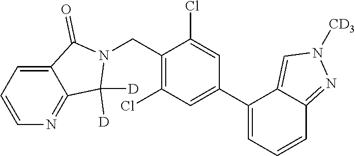 [SMILES: [2H]C1([2H])c2ncccc2C(=O)N1Cc1c(Cl)cc(-c2cccc3nn(C([2H])([2H])[2H])cc23)cc1Cl] | 6-(2,6-dichloro-4-(2- (methyl--d₃)-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.75 (dd, J = 4.9, 1.6 Hz, 1H), 8.59 (d, J = 1.0 Hz, 1H), 8.14 (dd, J = 7.7, 1.6 Hz, 1H), 7.84 (s, 2H), 7.67 (dd, J = 8.6, 1.0 Hz, 1H), 7.54 (dd, J = 7.7, 4.9 Hz, 1H), 7.34 (dd, J = 8.5, 7.0 Hz, 1H), 7.28 (dd, J = 7.0, 0.9 Hz, 1H), 5.10 (s, 2H); ES-MS [M + H]⁺ = 428.0. |
| 229 | 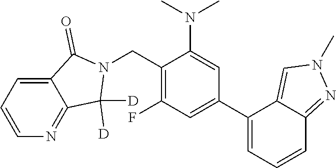 [SMILES: [2H]C1([2H])c2ncccc2C(=O)N1Cc1c(F)cc(-c2cccc3nn(C)cc23)cc1N(C)C] | 6-(2-(dimethylamino)-6- fluoro-4-(2-methyl-2H- indazol-4- yl)benzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.54 (s, 1H), 8.12 (dd, J = 7.7, 1.6 Hz, 1H), 7.62 (d, J = 8.6 Hz, 1H), 7.53 (dd, J = 7.7, 4.9 Hz, 1H), 7.36- 7.27 (m, 2H), 7.26-7.19 (m, 2H), 5.00- 4.91 (m, 2H), 4.19 (s, 2H), 2.77 (s, 6H); ES- MS [M + H]⁺ = 418.0. |
| 230 | 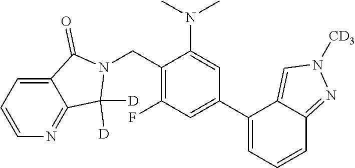 [SMILES: [2H]C1([2H])c2ncccc2C(=O)N1Cc1c(F)cc(-c2cccc3nn(C([2H])([2H])[2H])cc23)cc1N(C)C] | 6-(2-(dimethylamino)-6- fluoro-4-(2-(methyl-d₃)- 2H-indazol-4- yl)benzyl)- 6,7-dihydro-5H- pyrrolo[3,4-b]pyridin-5- one-7,7-d2 | ¹H NMR (400 MHz, DMSO-d₆) δ 8.74 (dd, J = 4.9, 1.6 Hz, 1H), 8.54 (d, J = 1.0 Hz, 1H), 8.12 (dd, J = 7.7, 1.6 Hz, 1H), 7.66- 7.59 (m, 1H), 7.53 (dd, J = 7.7, 4.9 Hz, 1H), 7.36-7.27 (m, 2H), 7.26-7.19 (m, 2H), 4.96 (d, J = 1.5 Hz, 2H), 2.77 (s, 5H); ES- MS [M + H]⁺ = 421.0. |
| 231 | 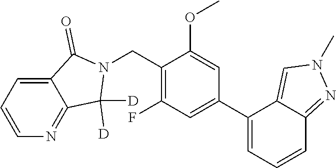 [SMILES: [2H]C1([2H])c2ncccc2C(=O)N1Cc1c(F)cc(-c2cccc3nn(C)cc23)cc1OC] | 6-(2-fluoro-6-methoxy- 4-(2-methyl-2H-indazol- 4-yl)benzyl)-6,7- dihydro-5H-pyrrolo[3,4- b]pyridin-5-one-7,7-d₂ | ¹H NMR (400 MHz, DMSO-d₆) δ 8.74 (dd, J = 4.9, 1.6 Hz, 1H), 8.60 (s, 1H), 8.10 (dd, J = 7.7, 1.6 Hz, 1H), 7.63 (d, J = 8.5 Hz, 1H), 7.52 (dd, J = 7.7, 4.9 Hz, 1H), 7.33 (dd, J = 8.5, 6.9 Hz, 1H), 7.26 (d, J = 6.8 Hz, 1H), 7.21-7.12 (m, 2H), 4.84 (s, 2H), 4.18 (s, 3H), 3.98 (s, 3H); ES-MS [M +H]⁺ = 405.0. |
| 232 | 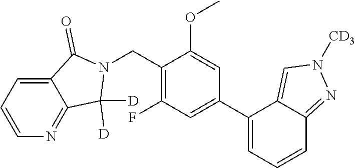 [SMILES: [2H]C1([2H])c2ncccc2C(=O)N1Cc1c(F)cc(-c2cccc3nn(C([2H])([2H])[2H])cc23)cc1OC] | 6-(2-fluoro-6-methoxy- 4-(2-(methyl-d₃)-2H- indazol-4- yl)benzyl)-6,7- dihydro-5H-pyrrolo[3,4- b]pyridin-5-one-7,7-d₂ | ¹H NMR (400 MHz, DMSO-d₆) δ 8.74 (dd, J = 5.0, 1.6 Hz, 1H), 8.60 (d, J = 1.0 Hz, 1H), 8.10 (dd, J = 7.7, 1.6 Hz, 1H), 7.66-7.59 (m, 1H), 7.52 (dd, J = 7.7, 4.9 Hz, 1H), 7.33 (dd, J = 8.5, 7.0 Hz, 1H), 7.26 (dd, J = 6.9, 0.9 Hz, 1H), 7.21-7.12 (m, 2H), 4.84 (d, J = 1.5 Hz, 2H), 3.98 (s, 3H); ES-MS [M + H]⁺ = 408.0. |
| 233 | 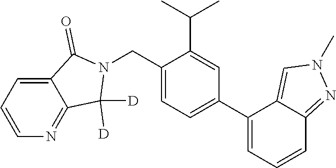 [SMILES: [2H]C1([2H])c2ncccc2C(=O)N1Cc1ccc(-c2cccc3nn(C)cc23)cc1C(C)C] | 6-(2-isopropyl-4-(2- methyl-2H-indazol-4- yl)benzyl)-6,7-dihydro- 5H-pyrrolo[3,4- b]pyridin-5-one-7,7-d₂ | ¹H NMR (400 MHz, CDCl₃) δ 8.72 (dd, J = 54.0, 1.6 Hz, 1H), 8.19 (dd, J = 7.7, 1.6 Hz, 1H), 8.00 (s, 1H), 7.71 (d, J = 8.7 Hz, 1H), 7.62 (d, J = 1.9 Hz, 1H), 7.49-7.37 (m, 3H), 7.34 (d, J = 7.8 Hz, 1H), 7.18 (dd, J = 6.9, 0.8 Hz, 1H), 4.98 (s, 2H), 4.27 (s, 3H), 3.45- 3.31 (m, 1H), 1.27 (d, J = 6.8 Hz, 6H); ES- MS [M + H]⁺ = 399.3. |
| 234 | 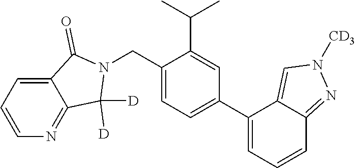 [SMILES: [2H]C1([2H])c2ncccc2C(=O)N1Cc1ccc(-c2cccc3nn(C([2H])([2H])[2H])cc23)cc1C(C)C] | 6-(2-isopropyl-4-(2- (methyl-d₃)-2H-indazol- 4-yl)benzyl)-6,7- dihydro-5H-pyrrolo[3,4- b]pyridin-5-one-7,7-d₂ | ¹H NMR (400 MHz, CDCl₃) δ 8.71 (dd, J = 5.0, 1.6 Hz, 1H), 8.17 (dd, J = 7.7, 1.6 Hz, 1H), 7.98 (d, J = 1.0 Hz, 1H), 7.69 (d, J = 8.7 Hz, 1H), 7.62 (d, J = 1.8 Hz, 1H), 7.47 (dd, J = 7.9, 1.9 Hz, 1H), 7.42 (dd, J = 7.7, 5.0 Hz, 1H), 7.39-7.31 (m, 2H), 7.16 (dd, J = 6.9, 0.8 Hz, 1H), 4.97 (s, 2H), 3.45- 3.31 (m, 1H), 1.26 (d, J = 6.8 Hz, 6H); ES-MS [M + H]⁺ = 402.2. |
| 235 | 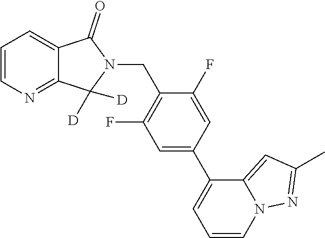 [SMILES: [2H]C1([2H])c2ncccc2C(=O)N1Cc1c(F)cc(-c2cccn3nc(C)cc23)cc1F] | 6-(2,6-difluoro-4-(2- methylpyrazolo[1,5- a]pyridin-4-yl)benzyl)- 6,7-dihydro-5H- pyrrolo[3,4-b]pyridin-5- one-7,7-d₂ | ¹H-NMR). ¹H-NMR (400 MHz, CDCl₃) δ 8.70 (dd, J = 4.9, 1.7 Hz, 1H), 8.37 (d, J = 6.9 Hz, 1H), 8.12 (dd, J = 7.7, 1.6 Hz, 1H), 7.38 (dd, J = 7.7, 4.9 Hz, 1H), 7.27-7.21 (m, 2H), 7.07 (dd, J = 7.0, 1.1 Hz, 1H), 6.74 (t, J = 7.0 Hz, 1H), 6.42 (s, 1H), 5.00 (s, 2H), 2.48 (s, 3H). ES-MS [M + H]⁺ = 393. |
| 236 | 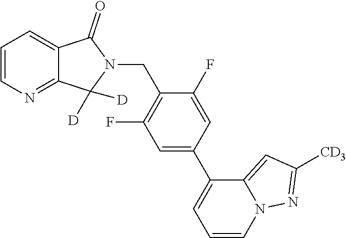 [SMILES: [2H]C([2H])([2H])c1cc2c(-c3cc(F)c(CN4C(=O)c5cccnc5C4([2H])[2H])c(F)c3)cccn2n1] | 6-(2,6-difluoro-4-(2- (methyl-d₃)pyrazolo[1,5- a]pyridin-4-yl)benzyl)- 6,7-dihydro-5H- pyrrolo[3,4-b]pyridin-5- one-7,7-d₂ | ¹H NMR (400 MHz, CDCl₃) δ 8.72 (dd, J = 4.9, 1.6 Hz, 1H), 8.40 (d, J = 7.0 Hz, 1H), 8.14 (dd, J = 7.7, 1.6 Hz, 1H), 7.40 (dd, J = 7.7, 5.0 Hz, 1H), 7.30-7.21 (m, 2H), 7.09 (dd, J = 7.1, 1.0 Hz, 1H), 6.76 (t, J = 7.0 Hz, 1H), 6.44 (d, J = 1.0 Hz, 1H), 5.01 (s, 2H). ES-MS [M + H]⁺ = 396. |
| 237 | 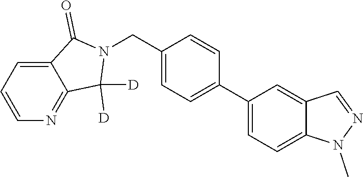 [SMILES: [2H]C1([2H])c2ncccc2C(=O)N1Cc1ccc(-c2ccc3c(cnn3C)c2)cc1] | 6-(4-(1-methyl-1H- indazol-5- yl)benzyl)-6,7- dihydro-5H-pyrrolo[3,4- b]pyridin-5-one-7,7-d₂ | ES-MS [M + H]⁺ = 357.4 |
| 238 | 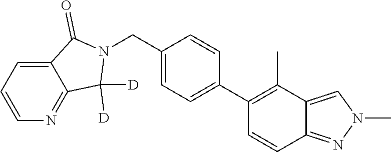 [SMILES: [2H]C1([2H])c2ncccc2C(=O)N1Cc1ccc(-c2ccc3nn(C)cc3c2C)cc1] | 6-(4-(2,4-dimethyl-2H- indazol-5- yl)benzyl)-6,7- dihydro-5H-pyrrolo[3,4- b]pyridin-5-one-7,7-d₂ | ES-MS [M + H]⁺ = 371.3 |
| 239 | 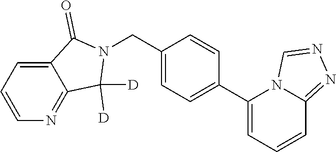 [SMILES: [2H]C1([2H])c2ncccc2C(=O)N1Cc1ccc(-c2cccc3nncn23)cc1] | 6-(4-([1,2,4]triazolo[4,3- a]pyridin-5-yl)benzyl)- 6,7-dihydro-5H- pyrrolo[3,4-b]pyridin-5- one-7,7-d₂ | ¹H NMR (400 MHz, CDCl₃) δ 8.74 (dd, J = 5.0, 1.6 Hz, 1H), 8.22-8.13 (m, 2H), 7.68- 7.60 (m, 2H), 7.53-7.48 (m, 2H), 7.48- 7.40 (m, 2H), 6.80 (dd, J = 9.1, 6.6 Hz, 1H), 6.47 (dd, J = 6.6, 1.1 Hz, 1H), 4.93 (s, 2H); ES-MS [M + H]⁺ = 343.3 |
| 240 | 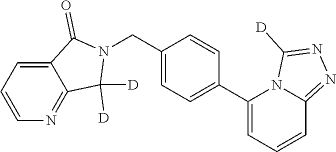 [SMILES: [2H]c1nnc2cccc(-c3ccc(CN4C(=O)c5cccnc5C4([2H])[2H])cc3)n12] | 6-(4-([1,2,4]triazolo[4,3- a]pyridin-5-yl-3- d)benzyl)-6,7-dihydro- 5H-pyrrolo[3,4- b]pyridin-5-one-7,7-d₂ | ES-MS [M + H]⁺ = 345.4 |
| 241 | 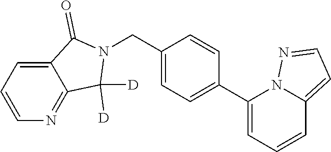 [SMILES: [2H]C1([2H])c2ncccc2C(=O)N1Cc1ccc(-c2cccc3ccnn23)cc1] | 6-(4-(pyrazolo[1,5- a]pyridin-7-yl)benzyl)- 6,7-dihydro-5H- pyrrolo[3,4-b]pyridin-5- one-7,7-d₂ | ES-MS [M + H]⁺ = 343.4 |
| 242 | 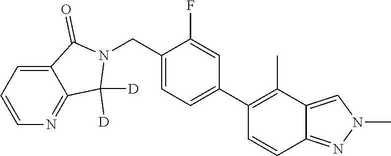 [SMILES: [2H]C1([2H])c2ncccc2C(=O)N1Cc1ccc(-c2ccc3nn(C)cc3c2C)cc1F] | 6-(4-(2,4-dimethyl-2H- indazol-5-yl)-2- fluorobenzyl)-6,7- dihydro-5H-pyrrolo[3,4- b]pyridin-5-one-7,7-d₂ | ¹H NMR (400 MHz, CDCl₃) δ 8.73 (dd, J = 5.0, 1.6 Hz, 1H), 8.16 (dd, J = 7.7, 1.6 Hz, 1H), 7.93 (s, 1H), 7.56 (dt, J = 8.8, 0.8 Hz, 1H), 7.45-7.37 (m, 2H), 7.16 (d, J = 8.9 Hz, 1H), 7.15-7.09 (m, 2H), 4.95 (s, 2H), 4.24 (s, 3H), 2.43 (s, 3H); ES-MS [M + H]⁺ = 389.4 |
| 243 | 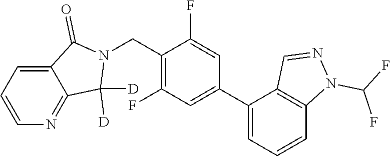 [SMILES: [2H]C1([2H])c2ncccc2C(=O)N1Cc1c(F)cc(-c2cccc3c2cnn3C(F)F)cc1F] | 6-(4-(1-(difluoro- methyl)-1H- indazol-4-yl)-2,6- difluorobenzyl)-6,7- dihydro-5H-pyrrolo[3,4- b]pyridin-5-one-7,7-d₂ | ES-MS [M + H]⁺ = 429.0 |
| 244 | 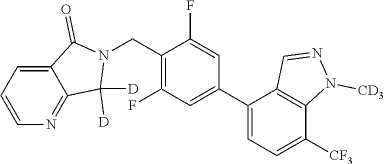 [SMILES: [2H]C1([2H])c2ncccc2C(=O)N1Cc1c(F)cc(-c2ccc(C(F)(F)F)c3c2cnn3C([2H])([2H])[2H])cc1F] | 6-(2,6-difluoro-4-(1- (methyl-d₃)-7- (trifluoromethyl)-1H- indazol-4- yl)benzyl)-6,7-di- hydro-5H-pyrrolo[3,4- b]pyridin-5-one-7,7-d₂ | ES-MS [M + H]⁺ = 464.0 |
| 245 | 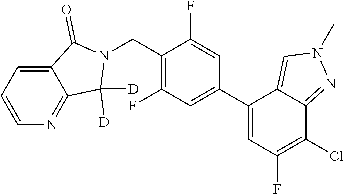 [SMILES: [2H]C1([2H])c2ncccc2C(=O)N1Cc1c(F)cc(-c2cc(F)c(Cl)c3nn(C)cc23)cc1F] | 6-(4-(7-chloro-6-fluoro- 2-methyl-2H-indazol-4- yl)-2,6-difluorobenzyl)- 6,7-dihydro-5H- pyrrolo[3,4-b]pyridin-5- one-7,7-d₂ | ES-MS [M + H]⁺ = 445.0 |
| 246 | 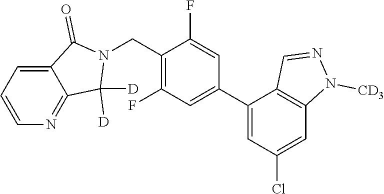 [SMILES: [2H]C1([2H])c2ncccc2C(=O)N1Cc1c(F)cc(-c2cc(Cl)cc3c2cnn3C([2H])([2H])[2H])cc1F] | 6-(4-(6-chloro-1- (methyl-d₃)-1H-indazol- 4-yl)-2,6- difluorobenzyl)-6,7- dihydro-5H-pyrrolo[3,4- b]pyridin-5-one-7,7-d₂ | ¹H NMR (400 MHz, DMSO) δ 8.76 (dd, J = 4.9, 1.6 Hz, 1H), 8.25 (d, J = 1.0 Hz, 1H), 8.12 (dd, J = 7.7, 1.6 Hz, 1H), 7.94 (dd, J = 1.6,1.0 Hz, 1H), 7.58-7.50 (m, 3H), 7.37 (d, J = 1.6 Hz, 1H), 4.92 (s, 2H); ES-MS [M + H]⁺ = 430.1 |
| 247 | 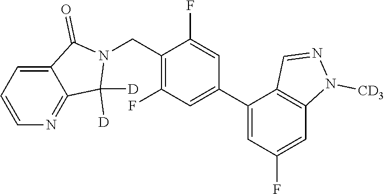 [SMILES: [2H]C1([2H])c2ncccc2C(=O)N1Cc1c(F)cc(-c2cc(F)cc3c2cnn3C([2H])([2H])[2H])cc1F] | 6-(2,6-difluoro-4-(6- fluoro-1-(methyl- d₃)-1H-indazol-4- yl)benzyl)-6,7-di- hydro-5H-pyrrolo[3,4- b]pyridin-5-one-7,7-d₂ | ¹H NMR (400 MHz, DMSO) δ 8.76 (dd, J = 4.9, 1.6 Hz, 1H), 8.24 (d, J = 1.0 Hz, 1H), 8.12 (dd, J = 7.7, 1.6 Hz, 1H), 7.64 (ddd, J = 9.4, 2.1, 1.0 Hz, 1H), 7.54 (td, J = 7.8, 4.8 Hz, 3H), 7.28 (dd, J = 10.2, 2.1 Hz, 1H), 4.92 (s, 2H); ES-MS [M + H]⁺ = 441.1 |
| 248 | 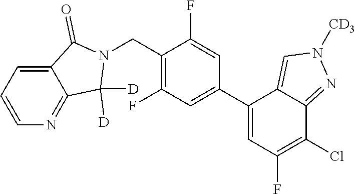 [SMILES: [2H]C1([2H])c2ncccc2C(=O)N1Cc1c(F)cc(-c2cc(F)c(Cl)c3nn(C([2H])([2H])[2H])cc23)cc1F] | 6-(4-(7-chloro-6-fluoro- 2-(methyl-d₃)-2H- indazol-4-yl)-2,6- difluorobenzyl)-6,7- dihydro-5H-pyrrolo[3,4- b]pyridin-5-one-7,7-d₂ | ¹H NMR (400 MHz, DMSO) δ 8.82 (s, 1H), 8.76 (dd, J = 4.9, 1.6 Hz, 1H), 8.12 (dd, J = 7.7, 1.6 Hz, 1H), 7.59-7.50 (m, 3H), 7.41 (d, J = 10.8 Hz, 1H), 4.92 (s, 2H); ES-MS [M + H]⁺ = 448.0 |
| 249 | 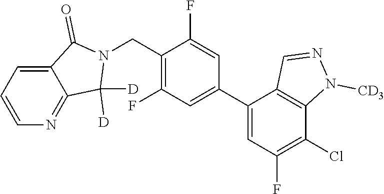 [SMILES: [2H]C1([2H])c2ncccc2C(=O)N1Cc1c(F)cc(-c2cc(F)c(Cl)c3c2cnn3C([2H])([2H])[2H])cc1F] | 6-(4-(7-chloro-6-fluoro- 1-(methyl-d₃)-1H- indazol-4-yl)-2,6- difluorobenzyl)-6,7- dihydro-5H-pyrrolo[3,4- b]pyridin-5-one-7,7-d₂ | ES-MS [M + H]⁺ = 448.0 |
| 250 | 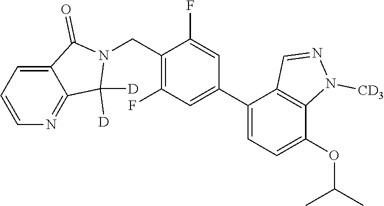 [SMILES: [2H]C1([2H])c2ncccc2C(=O)N1Cc1c(F)cc(-c2ccc(OC(C)C)c3c2cnn3C([2H])([2H])[2H])cc1F] | 6-(2,6-difluoro-4-(7- isopropoxy-1-(methyl- d₃)-1H-indazol-4- yl)benzyl)-6,7-dihydro- 5H-pyrrolo[3,4- b]pyridin-5-one-7,7-d₂ | ES-MS [M + H]⁺ = 454.1 |
| 251 | 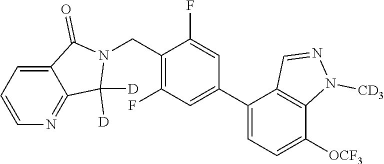 [SMILES: [2H]C1([2H])c2ncccc2C(=O)N1Cc1c(F)cc(-c2ccc(OC(F)(F)F)c3c2cnn3C([2H])([2H])[2H])cc1F] | 6-(2,6-difluoro-4-(1- (methyl-d₃)-7- (trifluoromethoxy)-1H- indazol-4- yl)benzyl)-6,7- dihydro-5H-pyrrolo[3,4- b]pyridin-5-one-7,7-d₂ | ES-MS [M + H]⁺ = 480.1 |
| 252 | 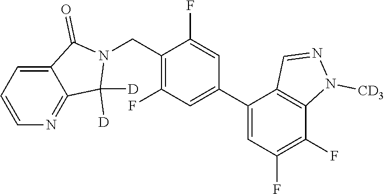 [SMILES: [2H]C1([2H])c2ncccc2C(=O)N1Cc1c(F)cc(-c2cc(F)c(F)c3c2cnn3C([2H])([2H])[2H])cc1F] | 6-(4-(6,7-difluoro-1- (methyl-d₃)-1H-indazol- 4-yl)-2,6- difluorobenzyl)-6,7- dihydro-5H-pyrrolo[3,4- b]pyridin-5-one-7,7-d₂ | ES-MS [M + H]⁺ = 432.2 |
| 253 | 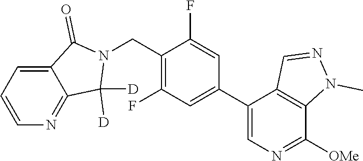 [SMILES: [2H]C1([2H])c2ncccc2C(=O)N1Cc1c(F)cc(-c2cnc(OC)c3c2cnn3C)cc1F] | 6-(2,6-difluoro-4-(7- methoxy-1-methyl-1H- pyrazolo[3,4-c]pyridin- 4-yl)benzyl)-6,7- dihydro-5H-pyrrolo[3,4- b]pyridin-5-one-7,7-d₂ | ES-MS [M + H]⁺ = 424.1. |

### Biological Activity

a. Cell Lines Expressing Muscarinic Acetylcholine Receptors

Chinese hamster ovary (CHO-K1) cells stably expressing rat (r)M₁ were purchased from the American Type Culture Collection and cultured according to their indicated protocol. CHO cells stably expressing human (h)M₂, hM₃, and hM₅ were described previously (Levey, et al., 1991); hM₁ and hM₄ cDNAs were purchased from Missouri S&T cDNA Resource; rM₄ cDNA was provided by T. I. Bonner (National Institutes of Health, Bethesda, MD). rM₂ and rM₃ were cloned from a rat brain cDNA library and sequence verified. hM₁, rM₂, rM₃, hM₄, and rM₄ cDNAs were used to stably transfect CHO-K1 cells purchased from the American Type Culture Collection using Lipofectamine2000. To make stable rM₂, hM₂, rM₃, hM₄, and rM₄ cell lines for use in calcium mobilization assays, these cells also were stably transfected with a chimeric G-protein (G_{qi5}) (provided by B. R. Conklin, University of California, San Francisco) using Lipofectamine 2000. rM₁, hM₁, rM₃, hM₃, rM₅, and hM₅ cells were grown in Ham's F-12 medium containing 10% heat-inactivated fetal bovine serum (FBS), 20 mM HEPES, and 50 μg/mL G418 sulfate. rM₂-G_{qi5}, hM₂-G_{qi5}, and hM₄-G_{qi5} cells were grown in the same medium also containing 500 μg/mL Hygromycin B. Stable rM₄-G_{qi5} cells were grown in DMEM containing 10% heat-inactivated FBS, 20 mM HEPES, 400 μg/mL G418 sulfate, and 500 μg/mL Hygromycin B.

b. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 and hygromycin at 15,000 cells/20 μL/well in Greiner 384-well black-walled, tissue culture (TC)-treated, clear-bottom plates (VWR). Cells were incubated overnight at 37° C. and 5% C₀₂. The next day, cells were washed using an ELX 405 (BioTek) with four washes (80 μL) of assay buffer then aspirated to 20 μL. Next, 20 μL of 16 μM Fluo-4/acetoxymethyl ester (Invitrogen, Carlsbad, CA) prepared as a 2.3 mM stock in DMSO and mixed in a 1:1 ratio with 10% (w/v) Pluronic F-127 and diluted in assay buffer was added to the wells and the cell plates were incubated for 50 min at 37° C. and 5% C₀₂. Dye was removed by washing with the ELX 405 (four 80 μL washes of assay buffer) then aspirated to 20 μL. Compound master plates were formatted in an 11 point CRC format (1:3 dilutions) in 100% DMSO with a starting concentration of 10 mM using the BRAVO liquid handler (Agilent). Test compound CRCs were then transferred to daughter plates (240 nL) using the Echo acoustic plate reformatter (Labcyte, Sunnyvale, CA) and then diluted into assay buffer (40 μL) to a 2× stock using a Thermo Fisher Combi (Thermo Fisher Scientific, Waltham, MA).

Calcium flux was measured using the Functional Drug Screening System (FDSS) 6000 (Hamamatsu Corporation, Tokyo, Japan) as an increase in the fluorescent static ratio. Compounds were applied to cells (20 μL, 2×) using the automated system of the FDSS 6000 at 4 s into the 300 s protocol and the data were collected at 1 Hz. At 144 s into the 300 s protocol, 10 μL of an EC₂₀ concentration of the muscarinic receptor agonist acetylcholine was added (5×), followed by the addition of 12 μL of an EC₈₀ concentration of acetylcholine at the 230 s time point (5×). Agonist activity was analyzed as a concentration-dependent increase in calcium mobilization upon compound addition. Eₘₐₓ values for agonist activity are expressed relative to the maximum for acetylcholine. Positive allosteric modulator activity was analyzed as a concentration-dependent increase in the EC₂₀ acetylcholine response. Antagonist activity was analyzed as a concentration-dependent decrease in the EC₈₀ acetylcholine response. Concentration-response curves were generated using a four-parameter logistical equation in XLfit curve fitting software (IDBS, Bridgewater, NJ) for Excel (Microsoft, Redmond, WA) or Prism (GraphPad Software, Inc., San Diego, CA).

The above described assay was also operated in a second mode where an appropriate fixed concentration of the present compounds were added to the cells after establishment of a fluorescence baseline for about 3 seconds, and the response in cells was measured. 140 s later the appropriate concentration of agonist was added and readings taken for an additional 106 s. Data were reduced as described above and the EC₅₀ values for the agonist in the presence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

c. Results and Discussion of Biological Activity Data

Activity (EC₅₀ and Eₘₐₓ) was determined in the mAChR M₁ cell-based functional assay as described above and the data are shown in Table 5. The compound numbers correspond to the compound numbers used in Table 4. The data in Table 5 demonstrate that the disclosed compounds are positive allosteric modulators of human mAChR M₁ and show high PAM activity for the human mAChR M₁ receptor(s).

TABLE 20
Biological Activity Data

| | |
|---|---|
| PAM Activity | Agonist Activity |

| | Human | | Human | |
|---|---|---|---|---|
| Cpd. | M₁ EC₅₀ | Eₘₐₓ | M₁ EC₅₀ | Eₘₐₓ |
| No. | (nM) | (%) | (nM) | (%) |
| 1 | 538 | 84 | Inactive | 8 |
| 2 | 980 | 57 | 7700 | 34 |
| 3 | 900 | 56 | 6920 | 31 |
| 4 | 1060 | 48 | Inactive | 2 |
| 5 | 977 | 40 | Inactive | 2 |
| 6 | 4090 | 48 | Inactive | 7 |
| 7 | 952 | 53 | Inactive | 4 |
| 8 | 639 | 62 | Inactive | 4 |
| 9 | 686 | 71 | Inactive | 6 |
| 10 | 1460 | 62 | Inactive | 4 |
| 11 | 1300 | 77 | Inactive | 6 |
| 12 | 1230 | 62 | Inactive | 2 |
| 13 | 5190 | 75 | Inactive | 3 |
| 14 | 6800 | 77 | Inactive | 2 |
| 15 | 1770 | 51 | Inactive | 2 |
| 16 | >10,000 | 55 | Inactive | 3 |
| 17 | 853 | 79 | Inactive | 4 |
| 18 | 4170 | 65 | Inactive | 2 |
| 19 | >10,000 | 58 | Inactive | 4 |
| 20 | 3970 | 75 | Inactive | 3 |
| 21 | 2420 | 76 | Inactive | 6 |
| 22 | 1940 | 74 | Inactive | 6 |
| 23 | 1130 | 76 | Inactive | 7 |
| 24 | 3600 | 69 | Inactive | 2 |
| 25 | 1070 | 76 | Inactive | 4 |
| 26 | 7580 | 58 | Inactive | 3 |
| 27 | 542 | 84 | Inactive | 4 |
| 28 | 535 | 80 | 6240 | 28 |
| 29 | 632 | 81 | 7860 | 33 |
| 30 | 1090 | 79 | >10,000 | 22 |
| 31 | 263 | 84 | 8600 | 28 |
| 32 | 594 | 86 | 5460 | 16 |
| 33 | 392 | 72 | >10,000 | 2 |
| 34 | 2000 | 82 | >10,000 | 3 |
| 35 | 2420 | 63 | >10,000 | 2 |
| 36 | 476 | 84 | Inactive | 5 |
| 37 | 2300 | 64 | Inactive | 3 |
| 38 | 436 | 69 | >10,000 | 2 |
| 39 | 112 | 85 | Inactive | 9 |
| 40 | 233 | 66 | Inactive | 1 |
| 41 | 604 | 65 | Inactive | 2 |
| 42 | 371 | 81 | >10,000 | 13 |
| 43 | 1170 | 86 | >10,000 | 2 |
| 44 | 413 | 78 | >10,000 | 28 |
| 45 | 411 | 73 | Inactive | 2 |
| 46 | 681 | 77 | Inactive | 9 |
| 47 | 900 | 76 | Inactive | 5 |
| 48 | 488 | 85 | >10,000 | 9 |
| 49 | 848 | 80 | Inactive | 2 |
| 50 | 332 | 85 | Inactive | 4 |
| 51 | 173 | 89 | >10,000 | 8 |
| 52 | 317 | 85 | Inactive | 2 |
| 53 | 325 | 84 | >10,000 | 9 |
| 54 | 382 | 82 | >10,000 | 5 |
| 55 | 275 | 88 | Inactive | 7 |
| 56 | 317 | 87 | Inactive | 5 |
| 57 | 288 | 87 | Inactive | 4 |
| 58 | 614 | 83 | Inactive | 4 |
| 59 | 222 | 59 | Inactive | 2 |
| 60 | 693 | 34 | Inactive | 4 |
| 61 | 1356 | 68 | Inactive | 8 |
| 62 | 108 | 67 | 4343 | 57 |
| 63 | 125 | 89 | Inactive | 3 |
| 64 | 1840 | 79 | Inactive | 2 |
| 65 | 1300 | 73 | >10,000 | 2 |
| 66 | 2171 | 74 | Inactive | 2 |
| 67 | 823 | 71 | Inactive | 2 |
| 68 | >10,000 | 64 | Inactive | 2 |
| 69 | 2194 | 68 | Inactive | 2 |
| 70 | 1220 | 65 | Inactive | 2 |
| 71 | 698 | 75 | Inactive | 1 |
| 72 | 472 | 75 | Inactive | 4 |
| 73 | 170 | 77 | Inactive | 1 |
| 74 | 592 | 81 | Inactive | 1 |
| 75 | 626 | 55 | Inactive | 1 |
| 76 | 58 | 78 | 3742 | 29 |
| 77 | 1349 | 47 | Inactive | 1 |
| 78 | 100 | 77 | Inactive | 6 |
| 79 | 793 | 79 | 10,000 | 16 |
| 80 | 2230 | 59 | Inactive | 1 |
| 81 | 159 | 48 | Inactive | 2 |
| 82 | 120 | 79 | 4926 | 25 |
| 83 | 244 | 80 | 6231 | 19 |
| 84 | 1240 | 74 | Inactive | 2 |
| 85 | 654 | 51 | Inactive | 2 |
| 86 | 237 | 46 | Inactive | 2 |
| 87 | 819 | 69 | Inactive | 11 |
| 88 | 2702 | 37 | Inactive | 2 |
| 89 | 88 | 46 | Inactive | 1 |
| 90 | 1703 | 72 | Inactive | 2 |
| 91 | 718 | 84 | 7743 | 22 |
| 92 | 2403 | 54 | Inactive | 2 |
| 93 | 131 | 76 | 3296 | 21 |
| 94 | 139 | 76 | 3542 | 19 |
| 95 | 1397 | 72 | Inactive | 3 |
| 96 | 211 | 80 | 7524 | 26 |
| 97 | 164 | 88 | 5937 | 25 |
| 98 | 286 | 76 | 8394 | 21 |
| 99 | 285 | 83 | 5798 | 25 |
| 100 | 214 | 85 | Inactive | 6 |
| 101 | 1412 | 75 | Inactive | 3 |
| 102 | 469 | 77 | Inactive | 9 |
| 103 | 1553 | 73 | Inactive | 2 |
| 104 | 1172 | 69 | Inactive | 2 |
| 105 | 195 | 27 | Inactive | 2 |
| 106 | 140 | 30 | Inactive | 2 |
| 107 | 227 | 74 | 5456 | 16 |
| 108 | 381 | 74 | Inactive | 8 |
| 109 | 167 | 73 | 5433 | 17 |
| 110 | 1443 | 71 | Inactive | 4 |
| 111 | 161 | 65 | Inactive | 2 |
| 112 | 76 | 63 | Inactive | 2 |
| 113 | 188 | 75 | 5866 | 41 |
| 114 | 162 | 77 | 8009 | 43 |
| 115 | 680 | 68 | Inactive | 6 |
| 116 | 287 | 72 | 9434 | 21 |
| 117 | 317 | 70 | 10,000 | 21 |
| 118 | 527 | 66 | 10,000 | 16 |
| 119 | 10,000 | 63 | Inactive | 3 |
| 120 | 2818 | 66 | Inactive | 2 |
| 121 | 2923 | 55 | Inactive | 3 |
| 122 | 789 | 77 | Inactive | 3 |
| 123 | 490 | 80 | 7926 | 11 |
| 124 | 243 | 74 | 8272 | 32 |
| 125 | 382 | 72 | 10,000 | 14 |
| 126 | 423 | 65 | 10,000 | 14 |
| 127 | 486 | 65 | 8398 | 21 |
| 128 | 428 | 63 | 6068 | 18 |
| 129 | 1760 | 65 | Inactive | 1 |
| 130 | 123 | 82 | 4560 | 52 |
| 131 | 381 | 80 | Inactive | 5 |
| 132 | 195 | 77 | 7934 | 47 |
| 133 | 486 | 79 | 10,000 | 35 |
| 134 | 701 | 75 | 10,000 | 23 |
| 135 | 405 | 78 | 7522 | 26 |
| 136 | 245 | 74 | 8424 | 28 |
| 137 | 256 | 74 | 7918 | 53 |
| 138 | 837 | 75 | 10,000 | 11 |
| 139 | 188 | 75 | 5043 | 36 |
| 140 | 288 | 72 | 8202 | 54 |
| 141 | 122 | 73 | 8018 | 50 |
| 142 | 215 | 81 | 6061 | 13 |
| 143 | 1269 | 74 | 10,000 | 15 |
| 144 | 751 | 75 | 10,000 | 18 |
| 145 | 1071 | 73 | 10,000 | 16 |
| 146 | 179 | 79 | 4638 | 28 |
| 147 | 1095 | 79 | 10,000 | 12 |
| 148 | 540 | 81 | 10,000 | 31 |
| 149 | 717 | 77 | 10,000 | 26 |
| 150 | 777 | 80 | 10,000 | 22 |
| 151 | 694 | 76 | 10,000 | 22 |
| 152 | 643 | 80 | 10,000 | 27 |
| 153 | 1340 | 79 | 10,000 | 15 |
| 154 | 1093 | 77 | 10,000 | 17 |
| 155 | 2475 | 82 | 8249 | 34 |
| 156 | 194 | 85 | 8157 | 33 |
| 157 | 456 | 81 | 10,000 | 16 |
| 158 | 382 | 80 | Inactive | 6 |
| 159 | 164 | 83 | 6241 | 31 |
| 160 | 396 | 83 | 10,000 | 13 |
| 161 | 353 | 81 | Inactive | 6 |
| 162 | 435 | 81 | Inactive | 2 |
| 163 | 239 | 81 | Inactive | 3 |
| 164 | 476 | 82 | Inactive | 3 |
| 165 | 643 | 93 | 10,000 | 16 |
| 166 | 318 | 82 | Inactive | 5 |
| 167 | 256 | 85 | Inactive | 2 |
| 168 | 1481 | 86 | Inactive | 6 |
| 169 | 630 | 81 | Inactive | 7 |
| 170 | 322 | 81 | Inactive | 9 |
| 171 | 337 | 81 | 10,000 | 31 |
| 172 | 354 | 82 | 10,000 | 27 |
| 173 | 1337 | 82 | Inactive | 2 |
| 174 | 193 | 86 | 8119 | 26 |
| 175 | 837 | 86 | 10,000 | 12 |
| 176 | 1324 | 60 | Inactive | 2 |
| 177 | 201 | 82 | 6116 | 27 |
| 178 | 277 | 79 | Inactive | 9 |
| 179 | 1117 | 55 | Inactive | 1 |
| 180 | 297 | 85 | Inactive | 9 |
| 181 | 475 | 82 | Inactive | 9 |
| 182 | 594 | 87 | Inactive | 3 |
| 183 | 535 | 86 | 10,000 | 13 |
| 184 | 512 | 87 | Inactive | 5 |
| 185 | 722 | 74 | Inactive | 6 |
| 186 | 391 | 72 | 7226 | 15 |
| 187 | 473 | 67 | Inactive | 1 |
| 188 | 2709 | 71 | Inactive | 9 |
| 189 | 629 | 72 | Inactive | 3 |
| 190 | 119 | 84 | 7516 | 37 |
| 191 | 1050 | 58 | Inactive | 2 |
| 192 | 137 | 84 | 5961 | 44 |
| 193 | 266 | 88 | 9828 | 11 |
| 194 | 437 | 83 | 3546 | 18 |
| 195 | 198 | 79 | 3733 | 43 |
| 196 | 437 | 83 | 8676 | 43 |
| 197 | 433 | 85 | 10,000 | 37 |
| 198 | 347 | 79 | 5597 | 30 |
| 199 | 475 | 81 | Inactive | 4 |
| 200 | 768 | 83 | 10,000 | 15 |
| 201 | 332 | 83 | 5195 | 43 |
| 202 | 429 | 81 | 3238 | 44 |
| 203 | 815 | 75 | 2565 | 63 |
| 204 | 293 | 70 | 7453 | 49 |
| 205 | 248 | 86 | 5979 | 54 |
| 206 | 335 | 81 | 9700 | 38 |
| 207 | 673 | 84 | 8071 | 25 |
| 208 | 140 | 88 | 1195 | 59 |
| 209 | 769 | 83 | 2579 | 62 |
| 210 | 865 | 83 | Inactive | 14 |
| 211 | 1286 | 88 | Inactive | 3 |
| 212 | 1453 | 82 | Inactive | 2 |
| 213 | 1520 | 81 | Inactive | 3 |
| 214 | 1287 | 79 | Inactive | 2 |
| 215 | 2384 | 83 | Inactive | 2 |
| 216 | 3177 | 83 | Inactive | 2 |
| 217 | 207 | 78 | 2858 | 23 |
| 218 | 516 | 79 | 7763 | 25 |
| 219 | 490 | 89 | Inactive | 8 |
| 220 | 472 | 86 | Inactive | 8 |
| 221 | 2694 | 82 | Inactive | 2 |
| 222 | 3780 | 77 | Inactive | 1 |
| 223 | 5242 | 59 | Inactive | 1 |
| 224 | 6456 | 62 | Inactive | 1 |
| 225 | 1366 | 58 | Inactive | 3 |
| 226 | 1364 | 54 | Inactive | 2 |
| 227 | 900 | 63 | Inactive | 1 |
| 228 | 380 | 60 | Inactive | 1 |
| 229 | 10,000 | 42 | Inactive | 4 |
| 230 | 10,000 | 38 | Inactive | 3 |
| 231 | 806 | 73 | Inactive | 7 |
| 232 | 760 | 80 | Inactive | 8 |
| 233 | 10,000 | 43 | Inactive | 1 |
| 234 | 10,000 | 42 | Inactive | 1 |
| 235 | 744 | 73 | >10,000 | 23 |
| 236 | 745 | 76 | 9564 | 22 |
| 237 | >10,000 | 47 | Inactive | 2 |
| 238 | >10,000 | 39 | Inactive | 2 |
| 239 | >10,000 | 44 | Inactive | 3 |
| 240 | >10,000 | 27 | Inactive | 3 |
| 241 | >10,000 | 41 | Inactive | 3 |
| 242 | >10,000 | 43 | Inactive | 3 |
| 243 | >10,000 | 34 | Inactive | 5 |
| 244 | >10,000 | 34 | Inactive | 2 |
| 245 | >10,000 | 47 | Inactive | 1 |
| 246 | >10,000 | 25 | Inactive | 2 |
| 247 | >10,000 | 29 | Inactive | 2 |
| 248 | >10,000 | 34 | Inactive | 2 |
| 249 | >10,000 | 20 | Inactive | 14 |
| 250 | >10,000 | 51 | Inactive | 3 |
| 251 | >10,000 | 12 | Inactive | 2 |
| 252 | >10,000 | 15 | Inactive | 2 |
| 253 | >10,000 | 27 | Inactive | 2 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A compound of formula (I), or a pharmaceutically acceptable salt thereof, wherein 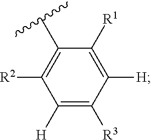
A¹ is 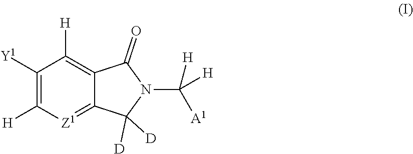
R¹ and R² are independently selected from the group consisting of H, D, C₁₋₄alkyl, CD₃, C₁₋₂fluoroalkyl, —OR^{2a}, OCD₃, halogen, C₃₋₅cycloalkyl, and —N(R^{2a})₂;
R^{2a}, at each occurrence, is independently selected from the group consisting of H, C₁₋₄alkyl, and C₁₋₄fluoroalkyl, wherein alternatively, two R^{2a}, together with a nitrogen to which the R^{2a} attach, form an azetidinyl, pyrrolidinyl, or piperidinyl;
R³ is a ring system selected from an aromatic or partially unsaturated 9-membered fused bicyclic heterocyclic ring system containing 1-3 nitrogen atoms, the heterocyclic ring system being unsubstituted or substituted with 1-4 substituents independently selected from the group consisting of halogen, C₁₋₆alkyl, C₁₋₆deuterioalkyl, C₁₋₆haloalkyl, oxo, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl;
R^{3a} is C₁₋₆alkyl or C₁₋₆deuterioalkyl, each independently of each other unsubstituted or substituted with a substituent selected from the group consisting of —OR^{3b}, —N(R^{3b})₂—SR^{3b}, —SO₂R^{3c}, —NR^{3b}C(O)R^{3b}, and —C(O)N(R^{3b})₂; or
R^{3a} is selected from the group consisting of —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl, wherein each heterocyclyl is a 4 to 7-membered heterocyclyl containing 1-2 heteroatoms independently selected from the group consisting of O, N, and S, wherein the —C₃₋₆cycloalkyl, —C₁₋₄alkylene-C₃₋₆cycloalkyl, —C₁₋₄alkylene-phenyl, -heterocyclyl, and —C₁₋₄alkylene-heterocyclyl are unsubstituted or substituted with 1-4 substituents independently selected from the group consisting of halogen, cyano, C₁₋₄alkyl, C₁₋₄deuterioalkyl, C₁₋₄haloalkyl, C₃₋₆cycloalkyl, oxo, —OR^{3b}, —N(R^{3b})₂, —NR^{3b}C(O)R^{3b}, —C(O)N(R^{3b})₂, —C(O)OR^{3b}, —SO₂R^{3c}, and C(O)R^{3b};
R^{3b}, at each occurrence, is independently selected from the group consisting of hydrogen, C₁₋₄alkyl, C₁₋₄deuterioalkyl, C₁₋₄haloalkyl, and C₃₋₄cycloalkyl;
R^{3c}, at each occurrence, is independently selected from the group consisting of C₁₋₄alkyl, C₁₋₄deuterioalkyl, C₁₋₄haloalkyl, and C₃₋₄cycloalkyl;
Y¹ is selected from the group consisting of H, CH₃, CD₃, CF₃, OH, OCH₃, OCD₃, and halogen; and
Z¹ is N or C—H.

2. The compound of claim 1, or a pharmaceutically acceptable salt thereof wherein A¹ is selected from the group consisting of

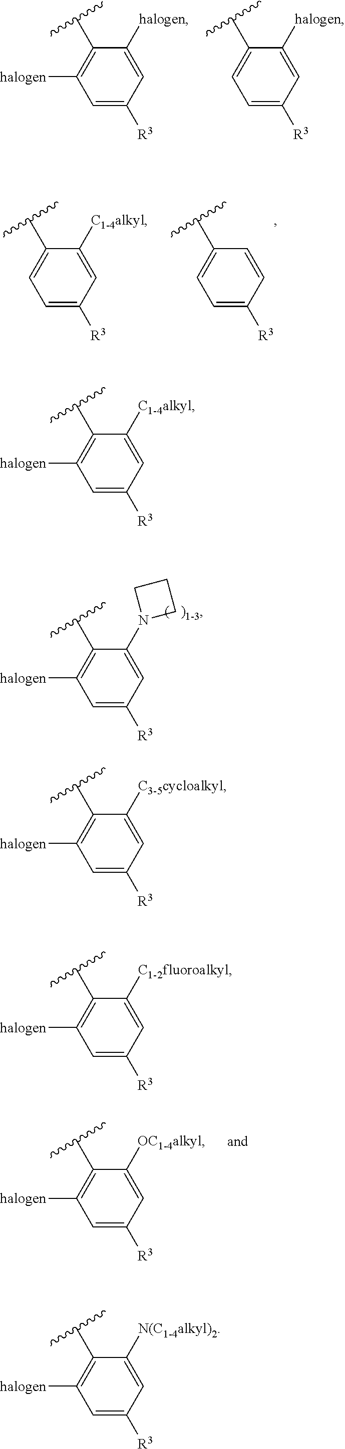

3. The compound of claim 2, or a pharmaceutically acceptable salt thereof, wherein A¹ is selected from the group consisting of

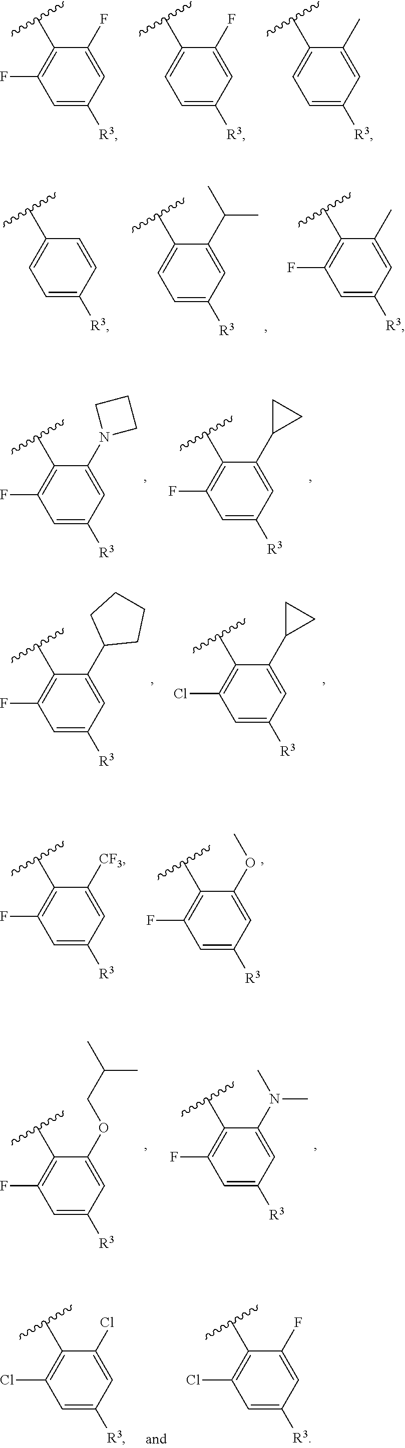

4. The compound of claim 3, or a pharmaceutically acceptable salt thereof, wherein:
A¹ is 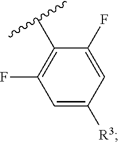
and/or
A¹ is 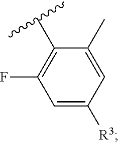
and/or
A¹ is 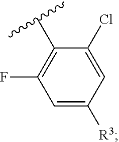
and/or
A¹ is

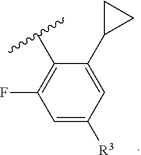

5. The compound of any of claims 1-4, or a pharmaceutically acceptable salt thereof, wherein the ring system of R³ is a 9-membered fused bicyclic heteroaryl containing 1-3 nitrogen atoms, the heteroaryl being unsubstituted or substituted as defined for R³ in claim 1.

6. The compound of claim 5, or a pharmaceutically acceptable salt thereof, wherein the ring system of the unsubstituted or substituted heteroaryl at R³ is selected from the group consisting of indazolyl, pyrazolo[3,4-b]pyridinyl, imidazo[1,2-a]pyridinyl, imidazo[1,5-a]pyridinyl, pyrazolo[3,4-c]pyridinyl, and pyrazolo[1,5-a]pyridinyl.

7. The compound of claim 6, or a pharmaceutically acceptable salt thereof, wherein the ring system of the unsubstituted or substituted heteroaryl at R³ is selected from the group consisting of indazol-4-yl, indazol-5-yl, indazol-6-yl, pyrazolo[3,4-b]pyridin-4-yl, pyrazolo[3,4-b]pyridin-5-yl, imidazo[1,2-a]pyridin-7-yl, imidazo[1,5-a]pyridin-6-yl, 2H-pyrazolo[3,4-c]pyridin-4-yl, and pyrazolo[1,5-a]pyridin-4-yl.

8. The compound of claim 5, or a pharmaceutically acceptable salt thereof, wherein R³ is selected from the group consisting of 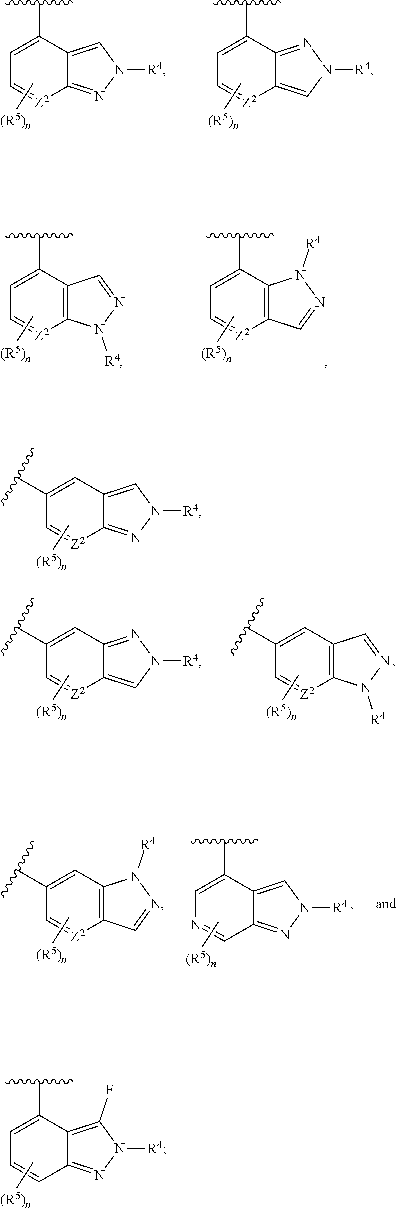
Z² is N or CR^{5a};
R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R⁵, at each occurrence, is independently selected from the group consisting of halogen, C₁₋₄alkyl, C₁₋₄haloalkyl, —OC₁₋₄alkyl, —OC₁₋₄deuterioalkyl, and —OC₁₋₄haloalkyl;
R^{5a} is H or R⁵; and
n is 0, 1, or 2.

9. The compound of claim 8, or a pharmaceutically acceptable salt thereof, wherein n is 0 or 1.

10. The compound of any of claims 1-6, or a pharmaceutically acceptable salt thereof, wherein R³ is selected from the group consisting of 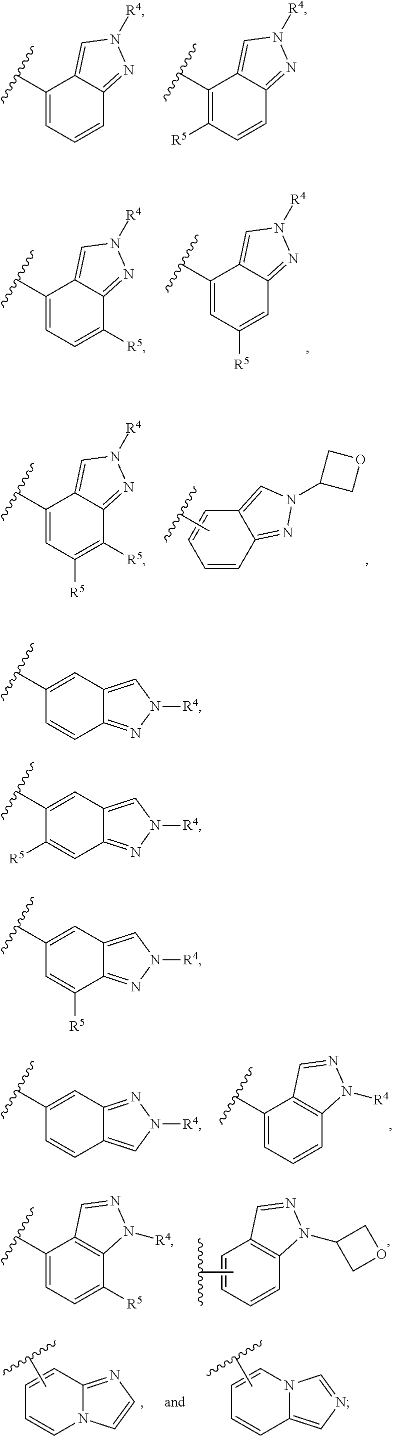
or
R³ is 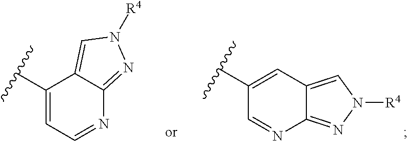
or
R³ is selected from the group consisting of 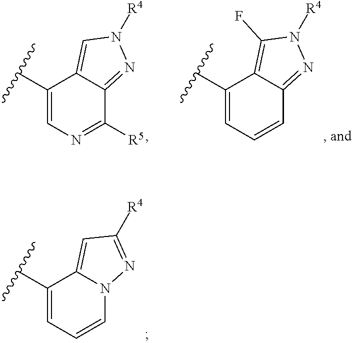
R⁴ is selected from the group consisting of H, methyl, ethyl, isopropyl, CH₂F, CHF₂, CD₃, and CD₂CD₃; and
R⁵, at each occurrence, is independently selected from the group consisting of fluoro, chloro, methyl, CHF₂, CF₃, OCH₃, OCD₃, OCHF₂, and OCF₃.

11. The compound of any of claims 1-7, or a pharmaceutically acceptable salt thereof, wherein:
R³ is 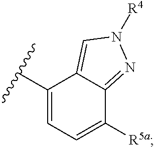
R⁴ is selected from the group consisting of H, C₁₋₄alkyl, C₁₋₄deuterioalkyl, C₁₋₄haloalkyl, C₃₋₄cycloalkyl, —C₁₋₃alkylene-C₃₋₄cycloalkyl, and oxetan-3-yl;
R^{5a} is H or R⁵; and
R⁵ is selected from the group consisting of halogen, C₁₋₆alkyl, C₁₋₆deuterioalkyl, C₁₋₆haloalkyl, —OH, —OR^{3a}, —OC₁₋₆haloalkyl, —OC₁₋₆halodeuterioalkyl, —SC₁₋₆alkyl, —S(O)(NH)C₁₋₆alkyl, C₃₋₅cycloalkyl, —C₁₋₃alkylene-C₃₋₅cycloalkyl, and oxetan-3-yl.

12. The compound of any of claims 8-9 or 11, or a pharmaceutically acceptable salt thereof, wherein R⁴ is selected from the group consisting of C₁₋₄alkyl, C₁₋₄deuterioalkyl, C₁₋₂fluoroalkyl, C₃₋₄cycloalkyl, —C₁₋₃alkylene-C₃₋₄cycloalkyl, and oxetan-3-yl.

13. The compound of any of claims 8-12, or a pharmaceutically acceptable salt thereof, wherein R⁴ is selected from the group consisting of methyl, ethyl, isopropyl, CH₂F, CHF₂, and CD₃.

14. The compound of any of claims 1-7, or a pharmaceutically acceptable salt thereof, wherein R³ is selected from the group consisting of

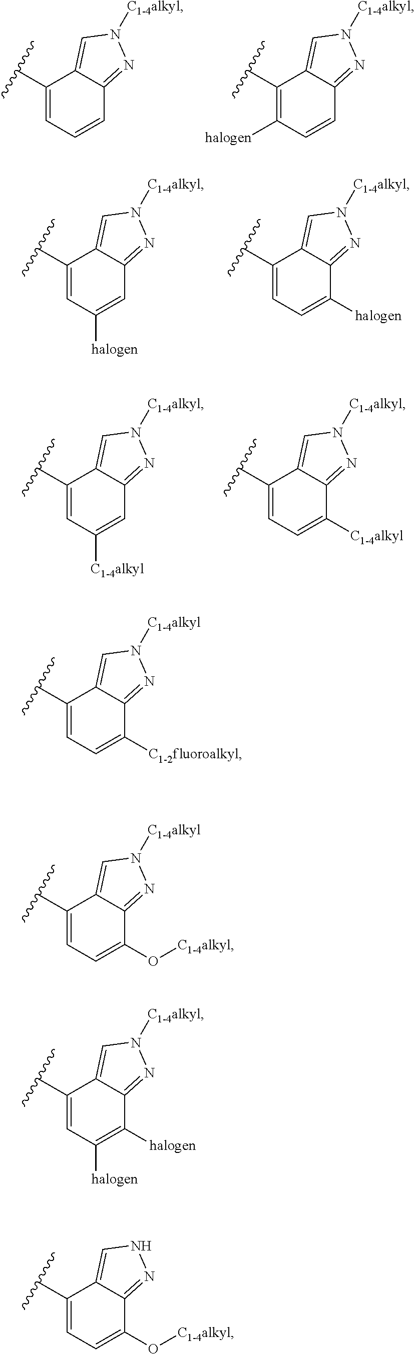

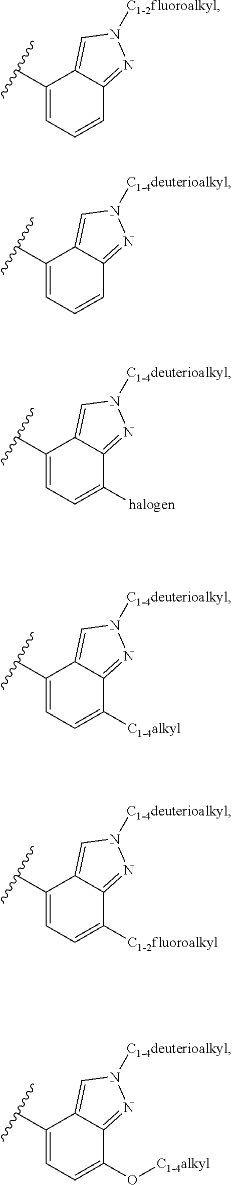

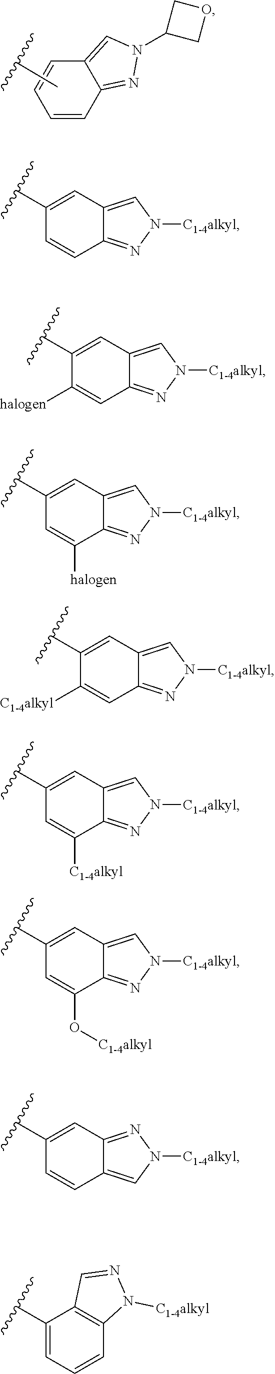

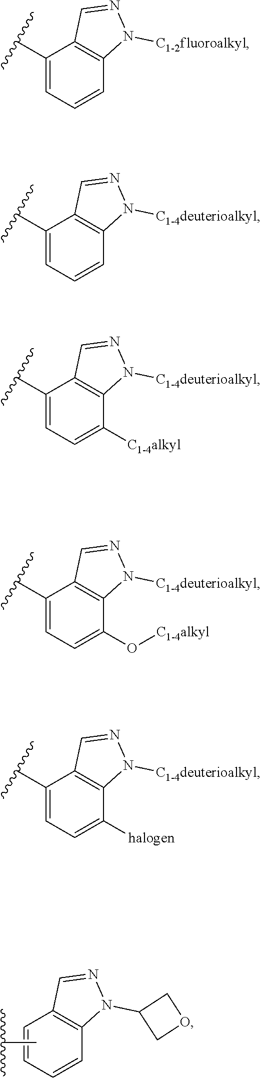

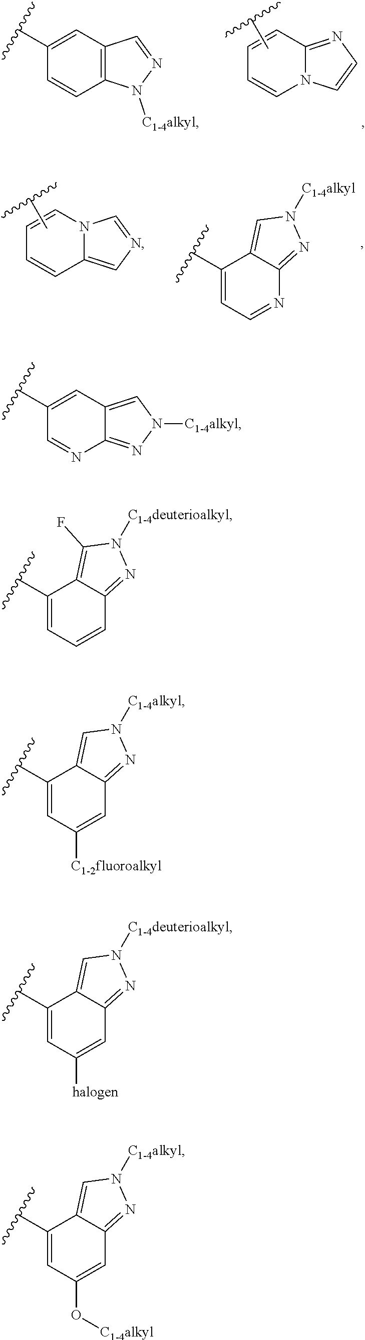

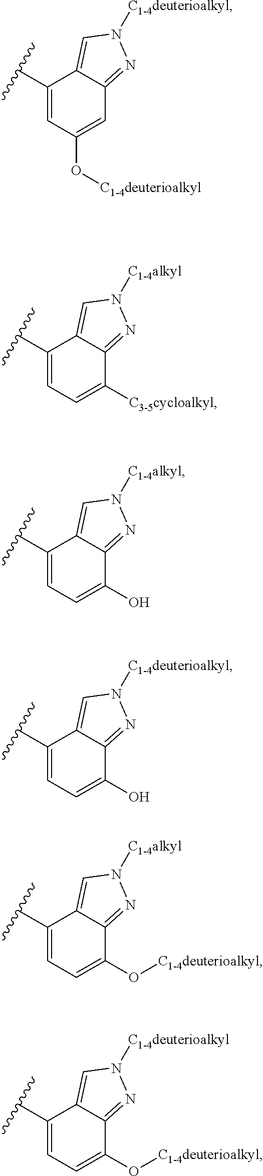

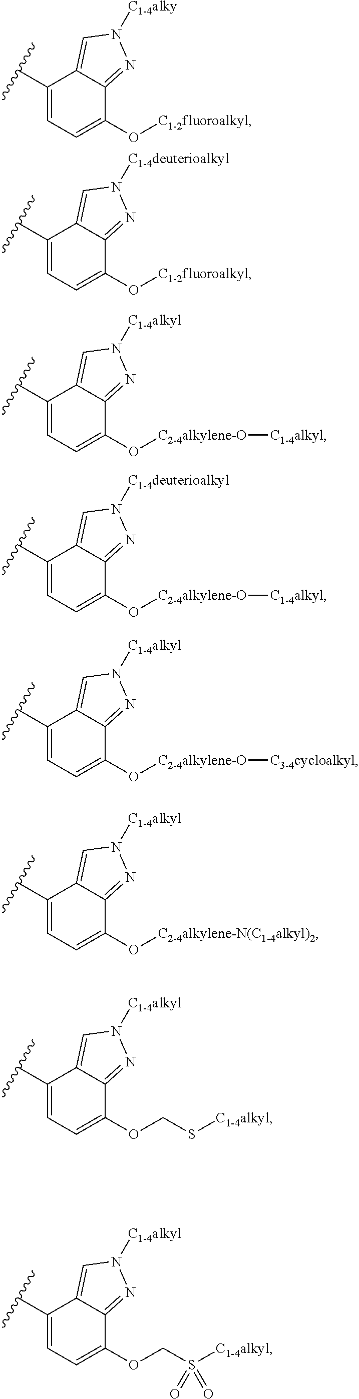

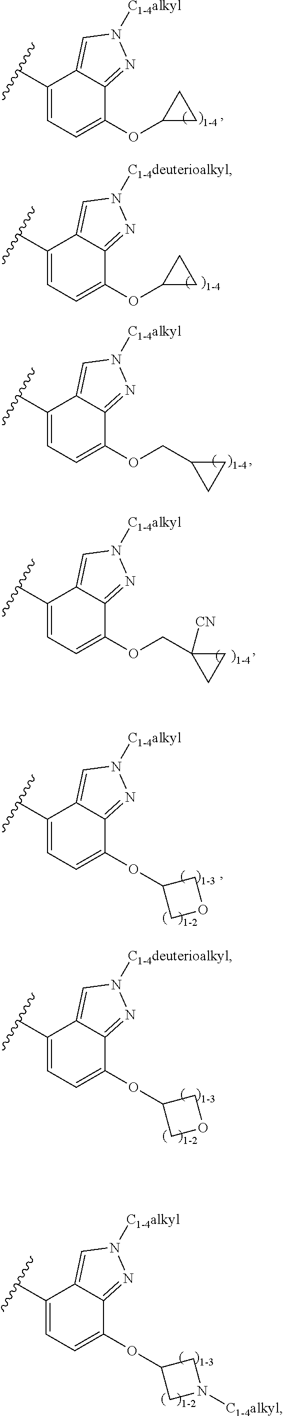

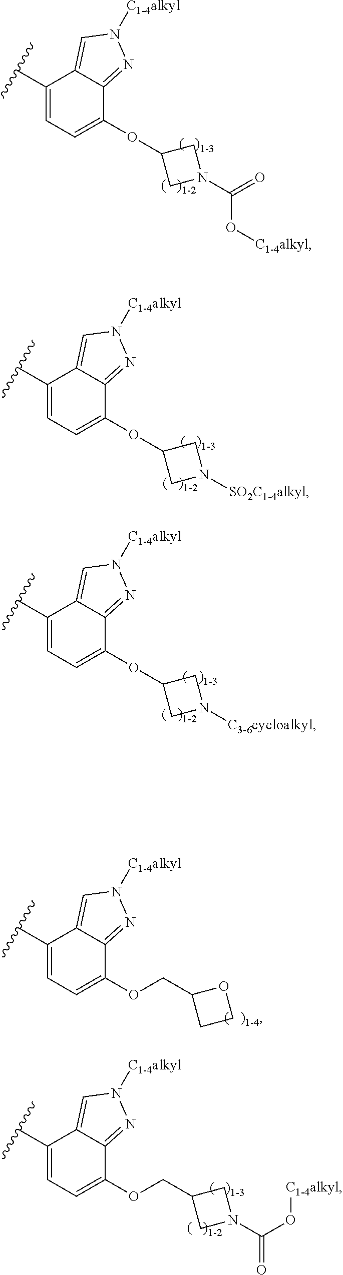

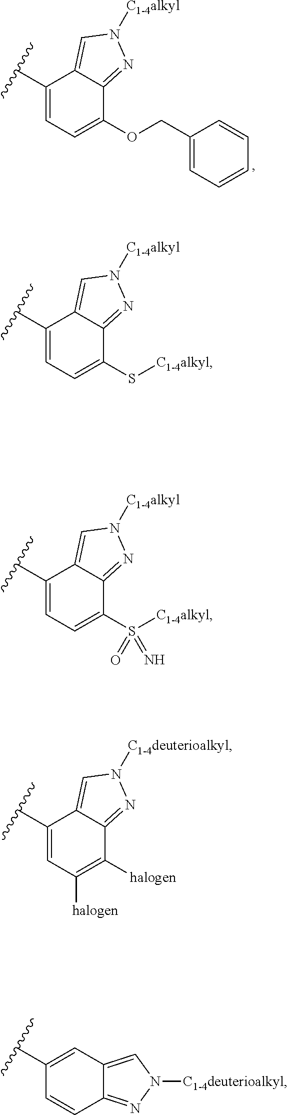

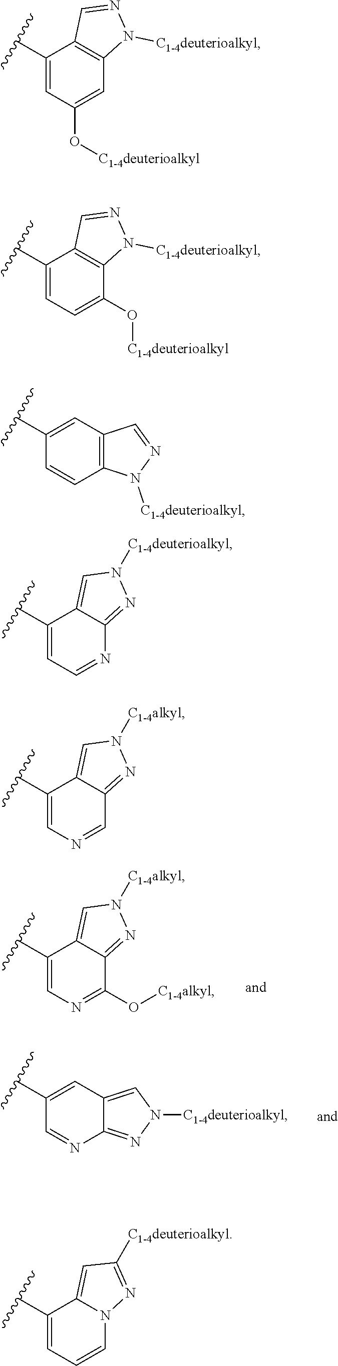

15. The compound of claim 14, or a pharmaceutically acceptable salt thereof, wherein R³ is selected from the group consisting of

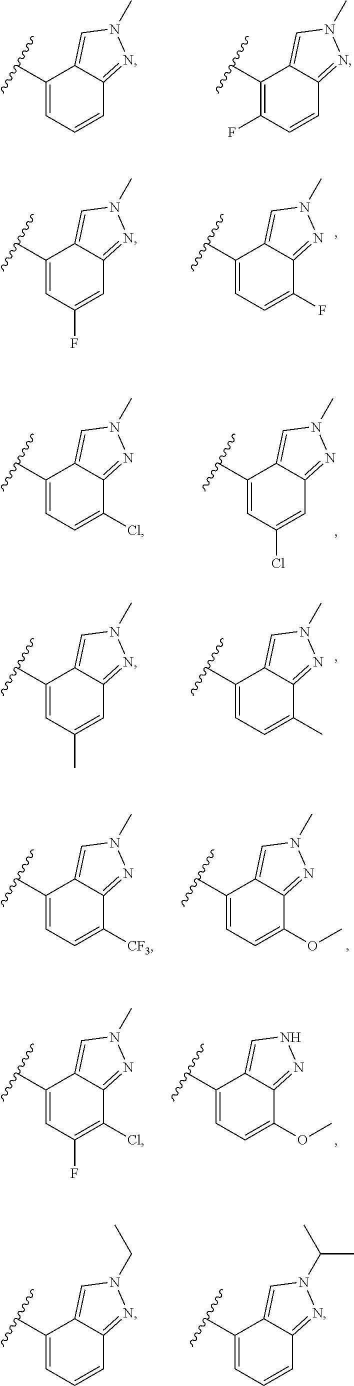

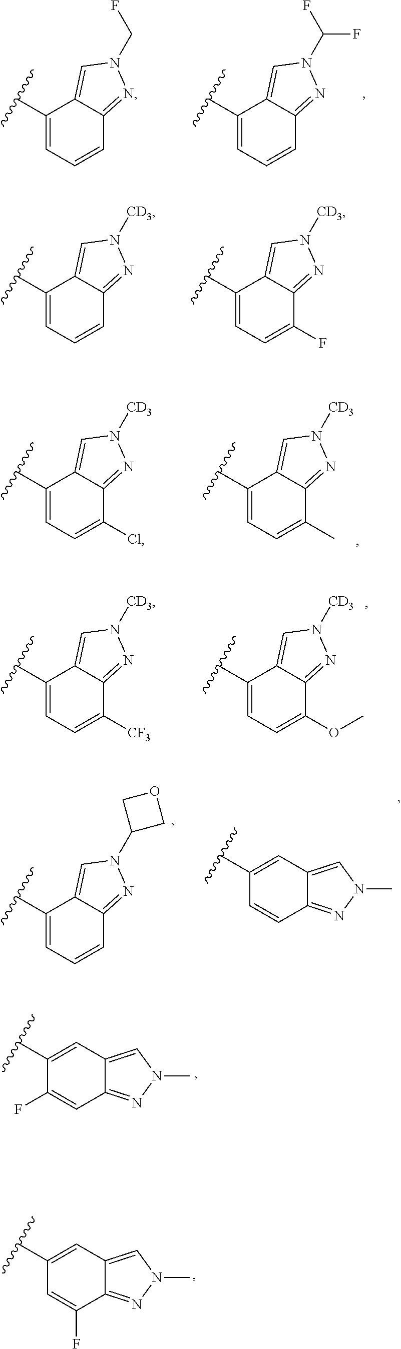

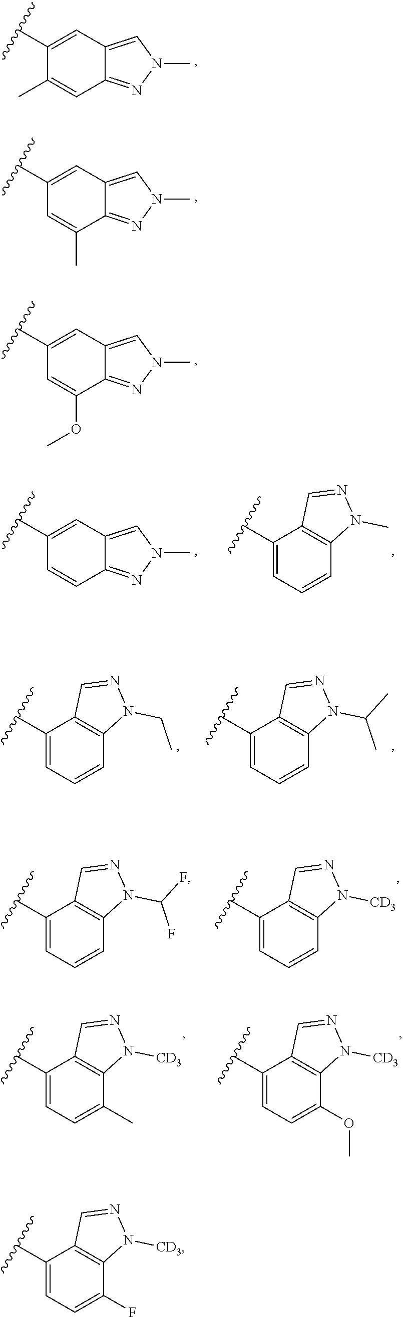

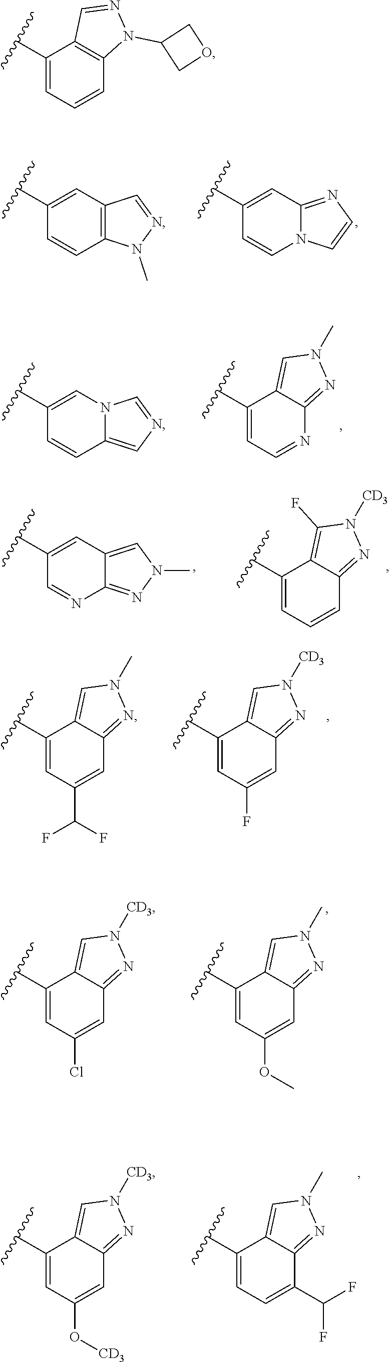

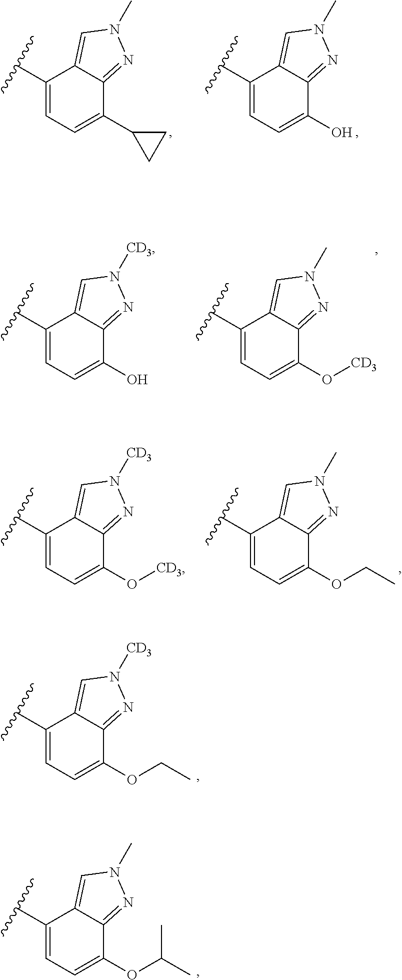

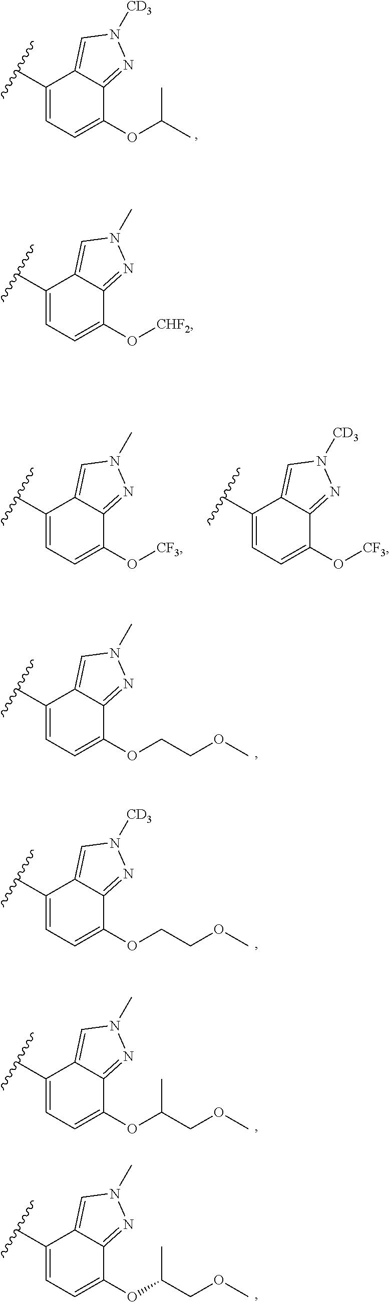

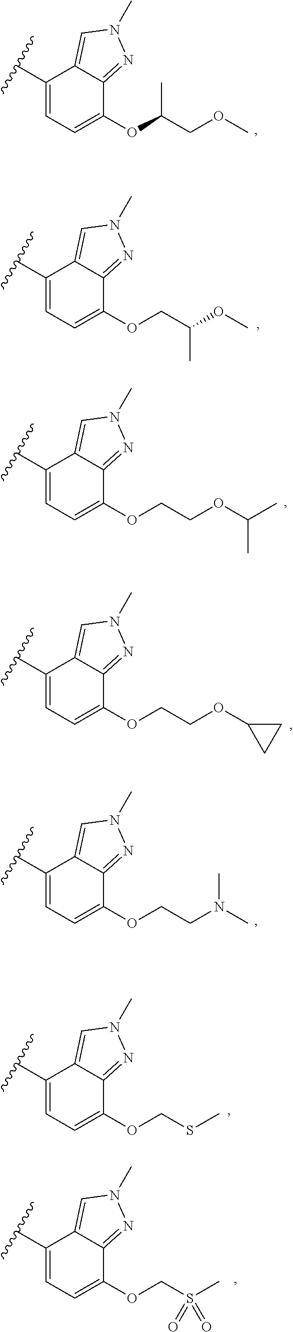

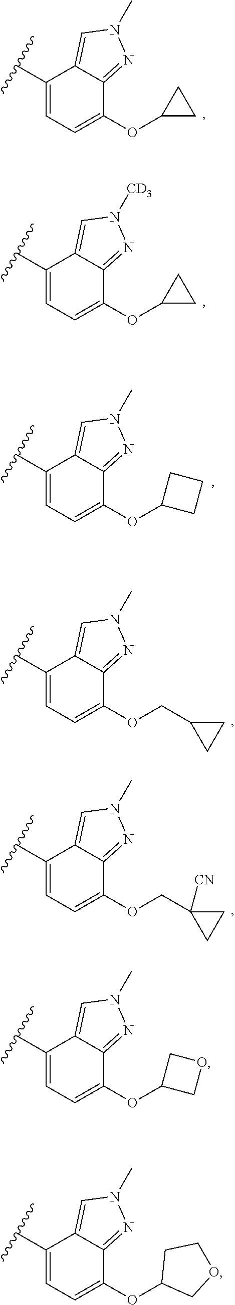

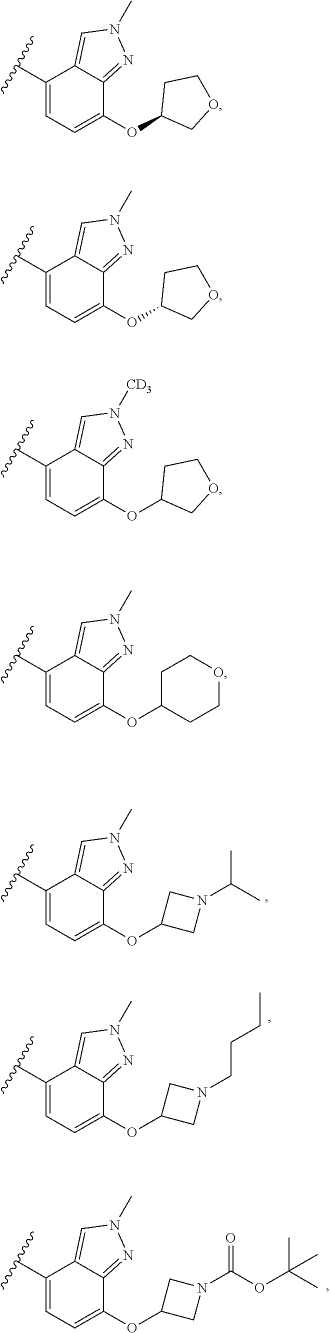

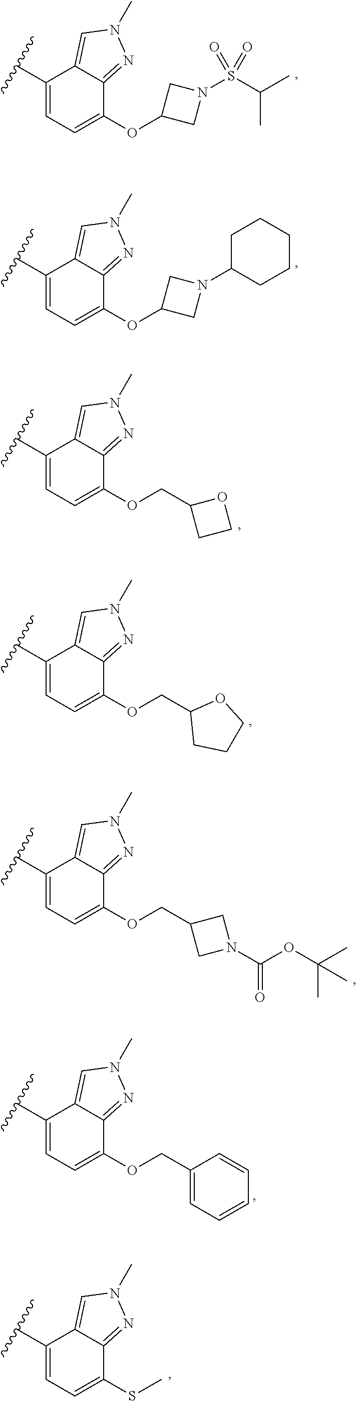

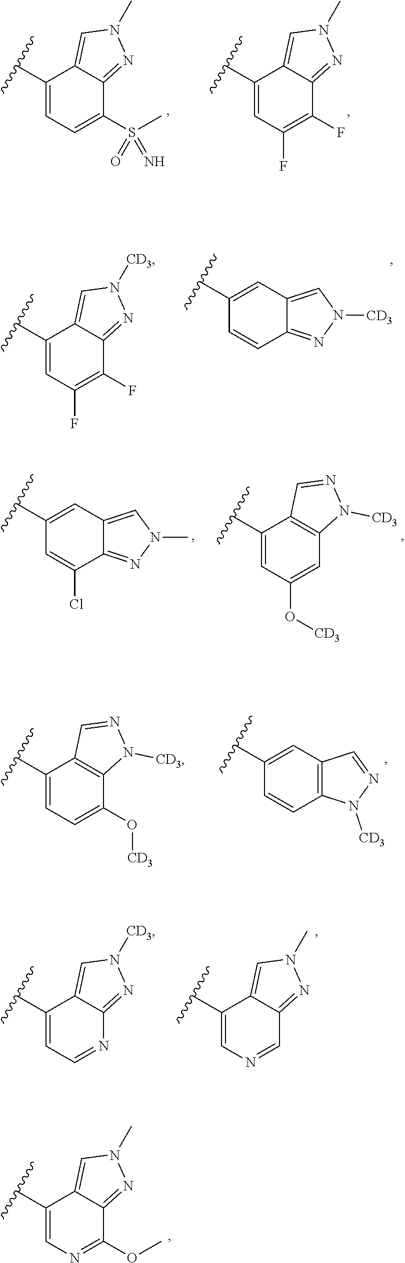

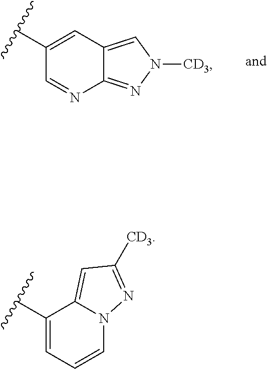 [SMILES: *c1cnc2nn(C([2H])([2H])[2H])cc2c1 ; *c1cccn2nc(C([2H])([2H])[2H])cc12]

16. The compound of any of claims 1-7, or a pharmaceutically acceptable salt thereof, wherein A¹ is selected from the group consisting of 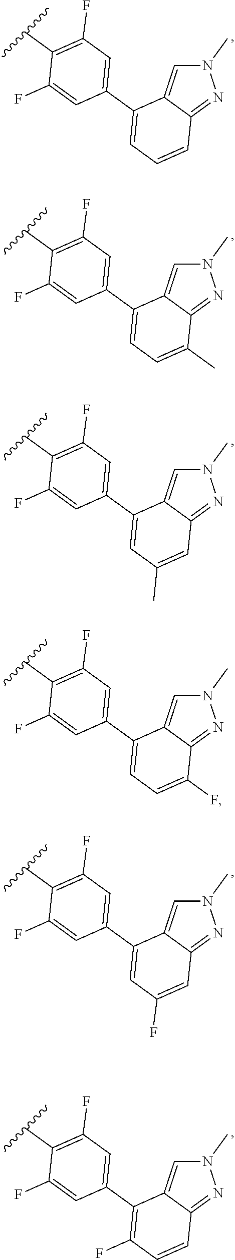 [SMILES: *c1c(F)cc(-c2cccc3nn(C)cc23)cc1F ; *c1c(F)cc(-c2ccc(C)c3nn(C)cc23)cc1F ; *c1c(F)cc(-c2cc(C)cc3nn(C)cc23)cc1F ; *c1c(F)cc(-c2ccc(F)c3nn(C)cc23)cc1F ; *c1c(F)cc(-c2cc(F)cc3nn(C)cc23)cc1F ; *c1c(F)cc(-c2c(F)ccc3nn(C)cc23)cc1F] 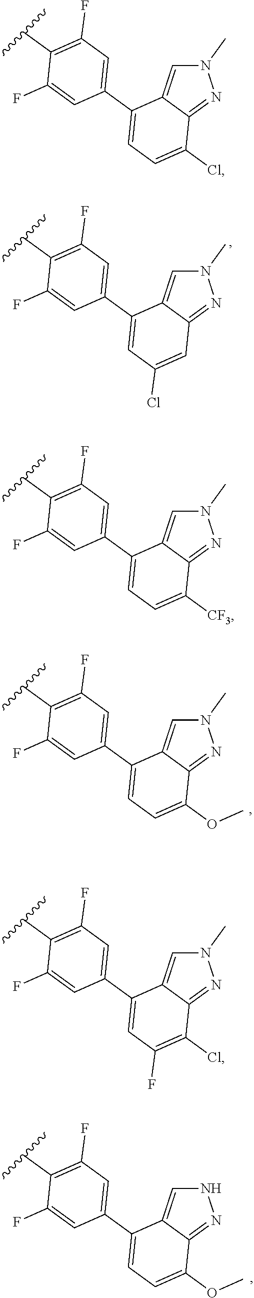 [SMILES: *c1c(F)cc(-c2ccc(Cl)c3nn(C)cc23)cc1F ; *c1c(F)cc(-c2cc(Cl)cc3nn(C)cc23)cc1F ; *c1c(F)cc(-c2ccc(C(F)(F)F)c3nn(C)cc23)cc1F ; *c1c(F)cc(-c2ccc(OC)c3nn(C)cc23)cc1F ; *c1c(F)cc(-c2cc(F)c(Cl)c3nn(C)cc23)cc1F ; *c1c(F)cc(-c2ccc(OC)c3n[nH]cc23)cc1F] 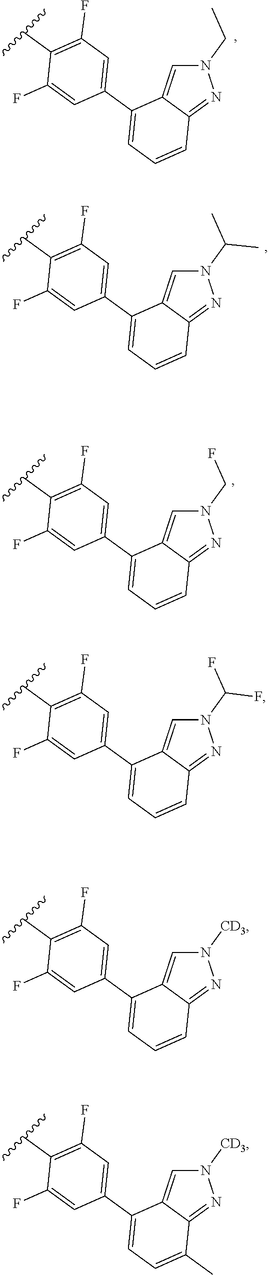 [SMILES: *c1c(F)cc(-c2cccc3nn(CC)cc23)cc1F ; *c1c(F)cc(-c2cccc3nn(C(C)C)cc23)cc1F ; *c1c(F)cc(-c2cccc3nn(CF)cc23)cc1F ; *c1c(F)cc(-c2cccc3nn(C(F)F)cc23)cc1F ; *c1c(F)cc(-c2cccc3nn(C([2H])([2H])[2H])cc23)cc1F ; *c1c(F)cc(-c2ccc(C)c3nn(C([2H])([2H])[2H])cc23)cc1F] 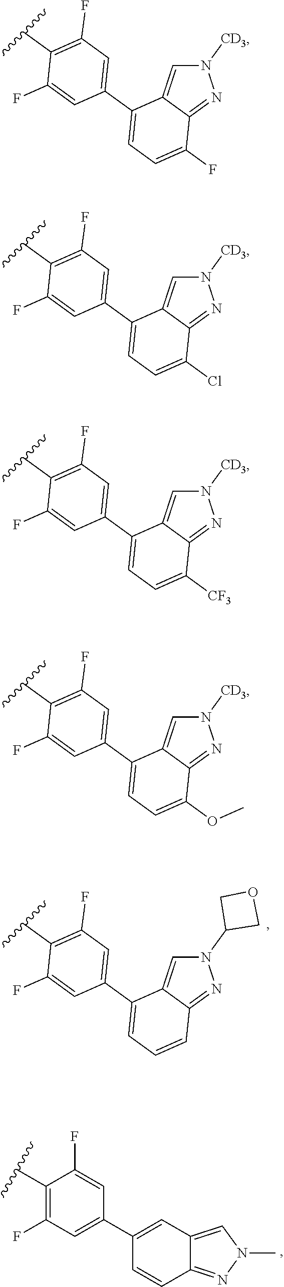 [SMILES: *c1c(F)cc(-c2ccc(F)c3nn(C([2H])([2H])[2H])cc23)cc1F ; *c1c(F)cc(-c2ccc(Cl)c3nn(C([2H])([2H])[2H])cc23)cc1F ; *c1c(F)cc(-c2ccc(C(F)(F)F)c3nn(C([2H])([2H])[2H])cc23)cc1F ; *c1c(F)cc(-c2ccc(OC)c3nn(C([2H])([2H])[2H])cc23)cc1F ; *c1c(F)cc(-c2cccc3nn(C4COC4)cc23)cc1F ; *c1c(F)cc(-c2ccc3nn(C)cc3c2)cc1F] 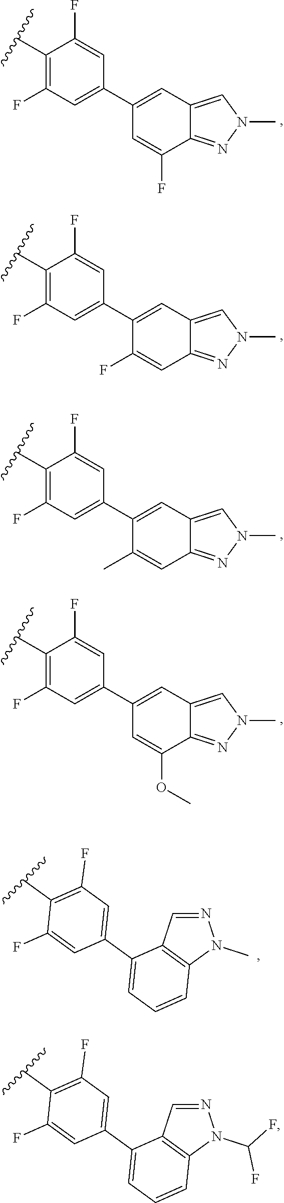 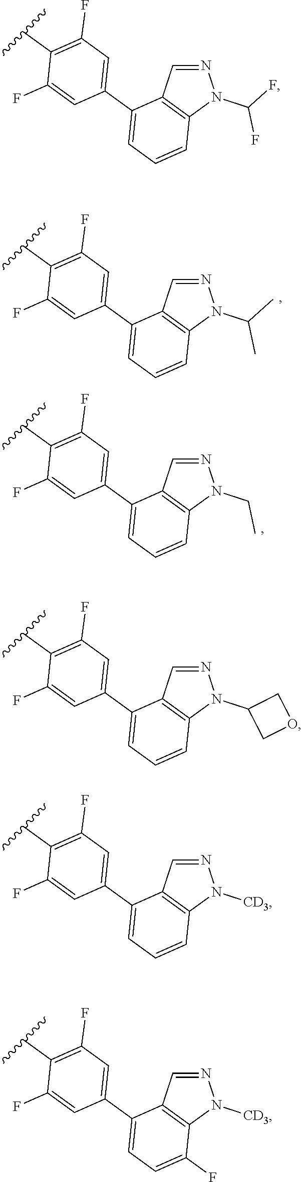 [SMILES: *c1c(F)cc(-c2cccc3c2cnn3C(F)F)cc1F ; *c1c(F)cc(-c2cccc3c2cnn3C(C)C)cc1F ; *c1c(F)cc(-c2cccc3c2cnn3CC)cc1F ; *c1c(F)cc(-c2cccc3c2cnn3C2COC2)cc1F ; *c1c(F)cc(-c2cccc3c2cnn3C([2H])([2H])[2H])cc1F ; *c1c(F)cc(-c2ccc(F)c3c2cnn3C([2H])([2H])[2H])cc1F] 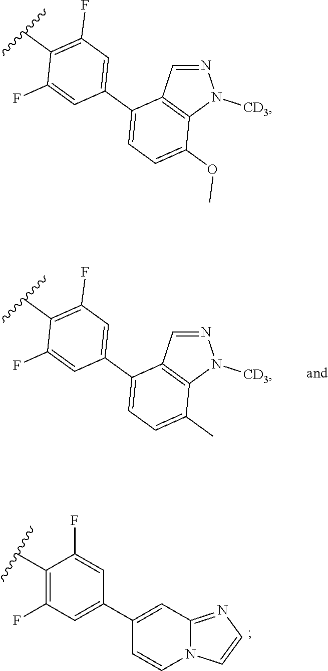 [SMILES: *c1c(F)cc(-c2ccc(OC)c3c2cnn3C([2H])([2H])[2H])cc1F ; *c1c(F)cc(-c2ccc(C)c3c2cnn3C([2H])([2H])[2H])cc1F ; *c1c(F)cc(-c2ccn3ccnc3c2)cc1F]
A¹ is selected from the group consisting of 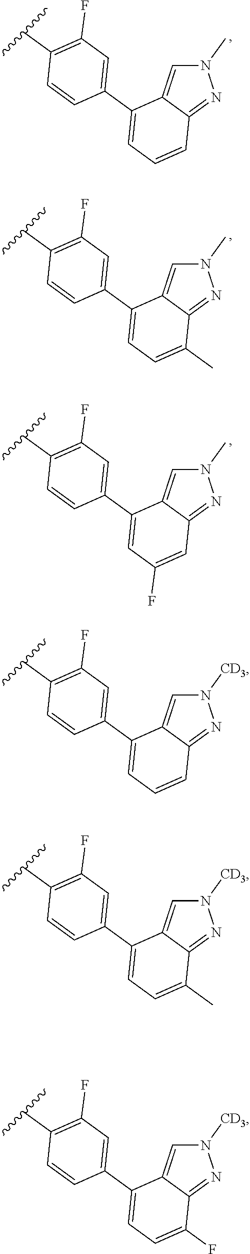 [SMILES: *c1ccc(-c2cccc3nn(C)cc23)cc1F ; *c1ccc(-c2ccc(C)c3nn(C)cc23)cc1F ; *c1ccc(-c2cc(F)cc3nn(C)cc23)cc1F ; *c1ccc(-c2cccc3nn(C([2H])([2H])[2H])cc23)cc1F ; *c1ccc(-c2ccc(C)c3nn(C([2H])([2H])[2H])cc23)cc1F ; *c1ccc(-c2ccc(F)c3nn(C([2H])([2H])[2H])cc23)cc1F] 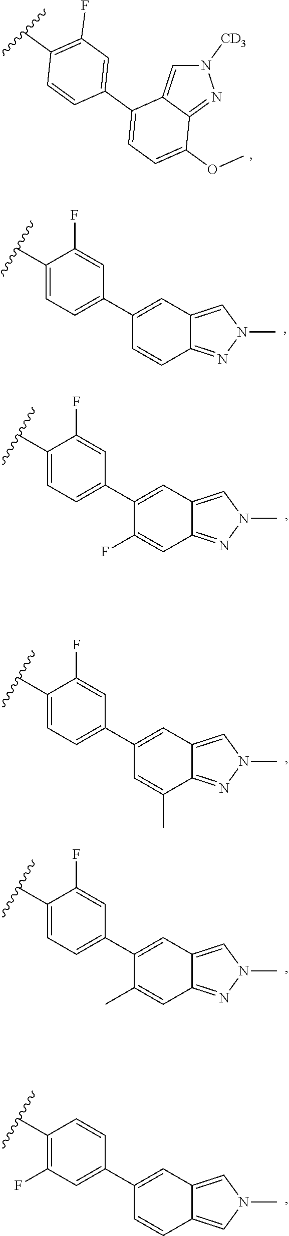 [SMILES: *c1ccc(-c2ccc(OC)c3nn(C([2H])([2H])[2H])cc23)cc1F ; *c1ccc(-c2ccc3nn(C)cc3c2)cc1F ; *c1ccc(-c2cc3cn(C)nc3cc2F)cc1F ; *c1ccc(-c2cc(C)c3nn(C)cc3c2)cc1F ; *c1ccc(-c2cc3cn(C)nc3cc2C)cc1F ; *c1ccc(-c2ccc3cn(C)cc3c2)cc1F] 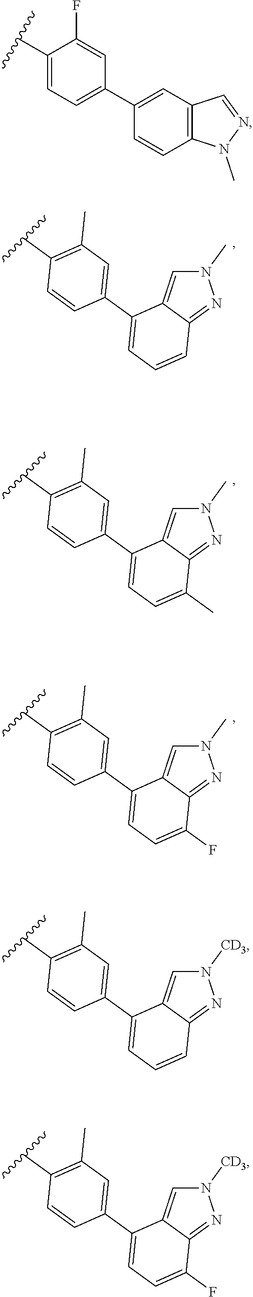 [SMILES: *c1ccc(-c2ccc3c(cnn3C)c2)cc1F ; *c1ccc(-c2cccc3nn(C)cc23)cc1C ; *c1ccc(-c2ccc(C)c3nn(C)cc23)cc1C ; *c1ccc(-c2ccc(F)c3nn(C)cc23)cc1C ; *c1ccc(-c2cccc3nn(C([2H])([2H])[2H])cc23)cc1C ; *c1ccc(-c2ccc(F)c3nn(C([2H])([2H])[2H])cc23)cc1C] 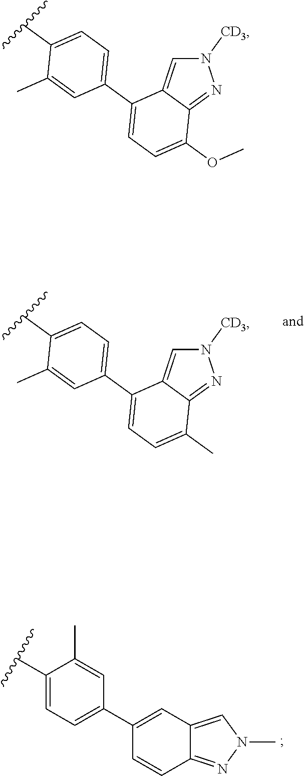
or
A¹ is selected from the group consisting of 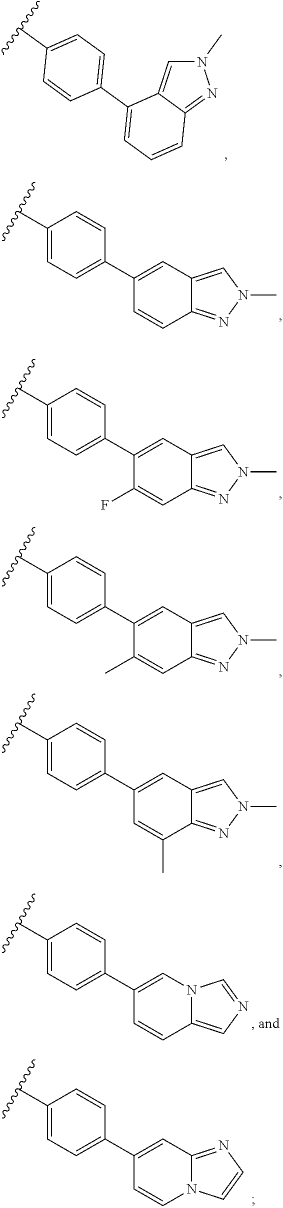
or
A¹ is 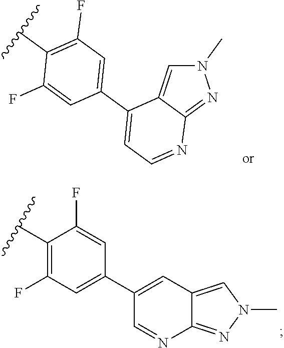 [SMILES: *c1c(F)cc(-c2ccnc3nn(C)cc23)cc1F ; *c1c(F)cc(-c2cnc3nn(C)cc3c2)cc1F]
or
A¹ is selected from the group consisting of 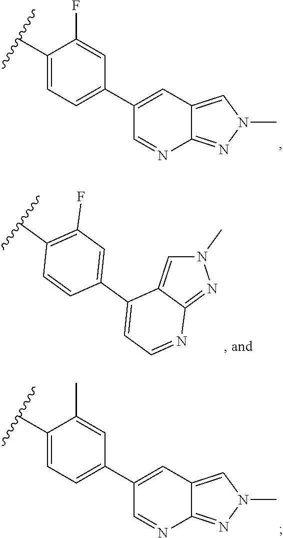 [SMILES: *c1ccc(-c2cnc3nn(C)cc3c2)cc1F ; *c1ccc(-c2ccnc3nn(C)cc23)cc1F ; *c1ccc(-c2cnc3nn(C)cc3c2)cc1C]
or
A¹ is

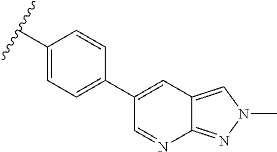 [SMILES: *c1ccc(-c2cnc3nn(C)cc3c2)cc1]

17. The compound of any one of claims 1-16, or a pharmaceutically acceptable salt thereof, wherein A¹ is selected from the group consisting of

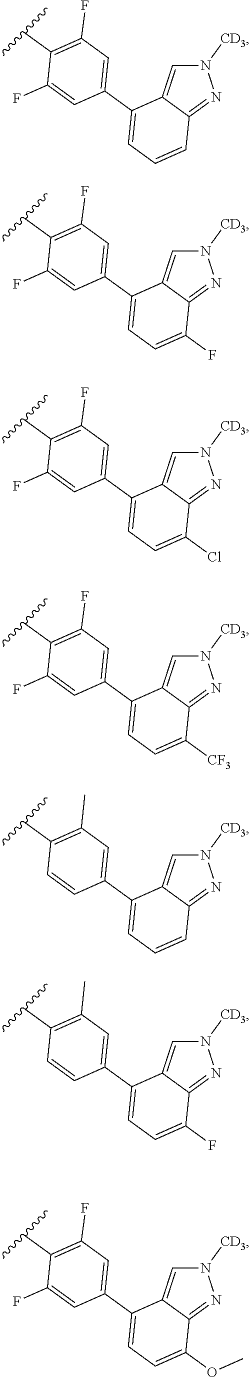

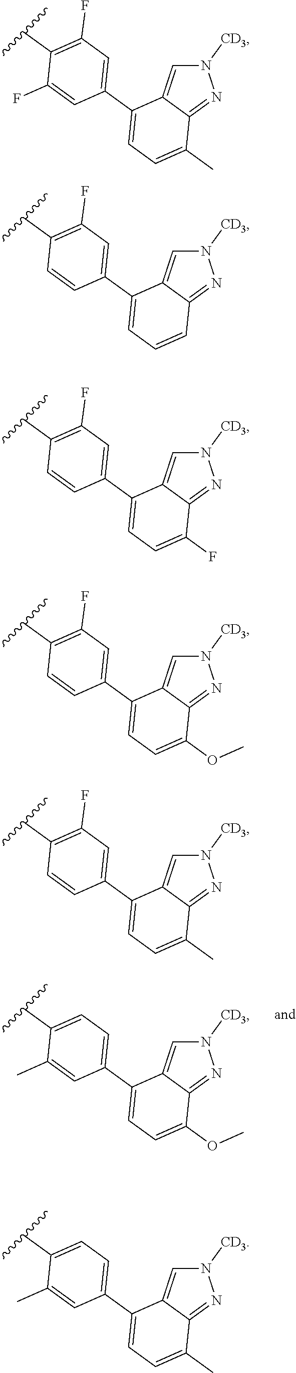

18. The compound of any of claims 1-17, or a pharmaceutically acceptable salt thereof, wherein A¹ is

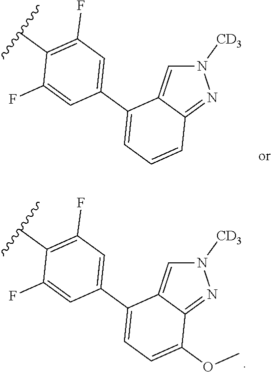 [SMILES: *c1c(F)cc(-c2cccc3nn(C([2H])([2H])[2H])cc23)cc1F ; *c1c(F)cc(-c2ccc(OC)c3nn(C([2H])([2H])[2H])cc23)cc1F]

19. The compound of any one of claims 1-16, or a pharmaceutically acceptable salt thereof, wherein A¹ is selected from the group consisting of

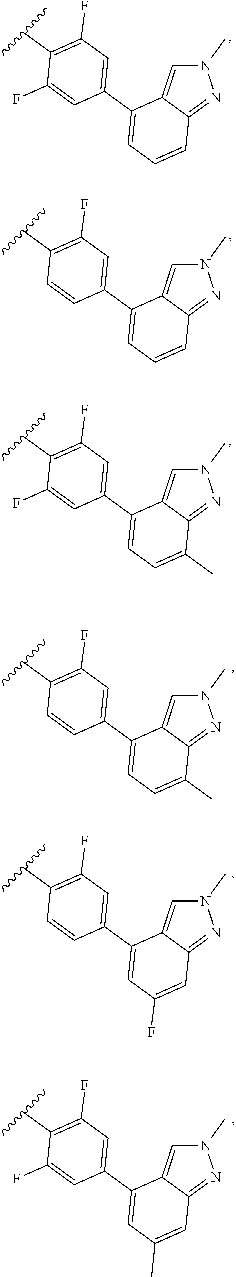 [SMILES: *c1c(F)cc(-c2cccc3nn(C)cc23)cc1F ; *c1ccc(-c2cccc3nn(C)cc23)cc1F ; *c1c(F)cc(-c2ccc(C)c3nn(C)cc23)cc1F ; *c1ccc(-c2ccc(C)c3nn(C)cc23)cc1F ; *c1ccc(-c2cc(F)cc3nn(C)cc23)cc1F ; *c1c(F)cc(-c2cc(C)cc3nn(C)cc23)cc1F]

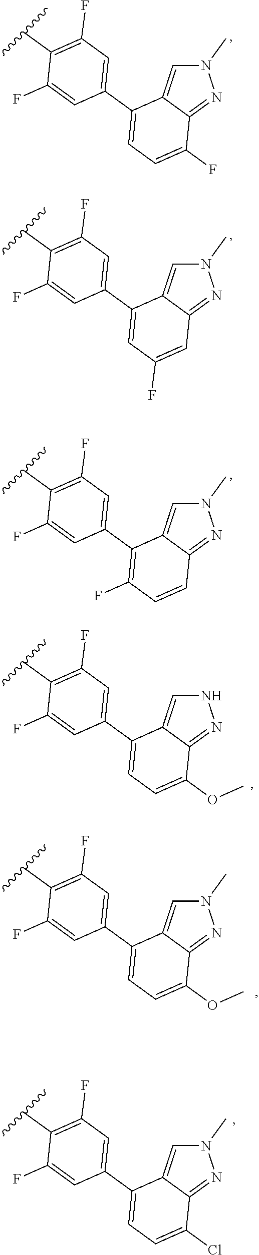

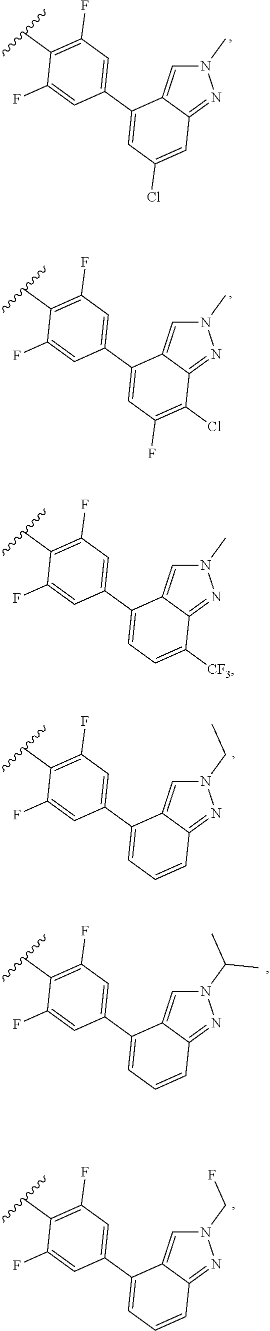 [SMILES: *c1c(F)cc(-c2cc(Cl)cc3nn(C)cc23)cc1F ; *c1c(F)cc(-c2cc(F)c(Cl)c3nn(C)cc23)cc1F ; *c1c(F)cc(-c2ccc(C(F)(F)F)c3nn(C)cc23)cc1F ; *c1c(F)cc(-c2cccc3nn(CC)cc23)cc1F ; *c1c(F)cc(-c2cccc3nn(C(C)C)cc23)cc1F ; *c1c(F)cc(-c2cccc3nn(CF)cc23)cc1F]

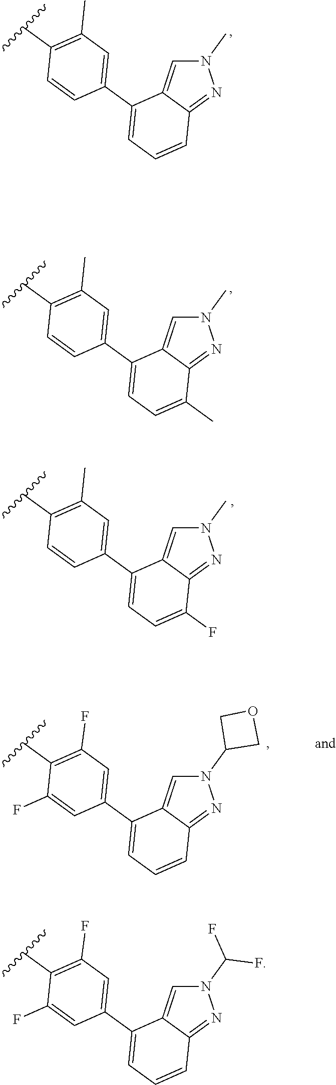 [SMILES: *c1ccc(-c2cccc3nn(C)cc23)cc1C ; *c1ccc(-c2ccc(C)c3nn(C)cc23)cc1C ; *c1ccc(-c2ccc(F)c3nn(C)cc23)cc1C ; *c1c(F)cc(-c2cccc3nn(C4COC4)cc23)cc1F ; *c1c(F)cc(-c2cccc3nn(C(F)F)cc23)cc1F]

20. The compound of any of claims 1-15, or a pharmaceutically acceptable salt thereof, wherein A¹ is selected from the group consisting of

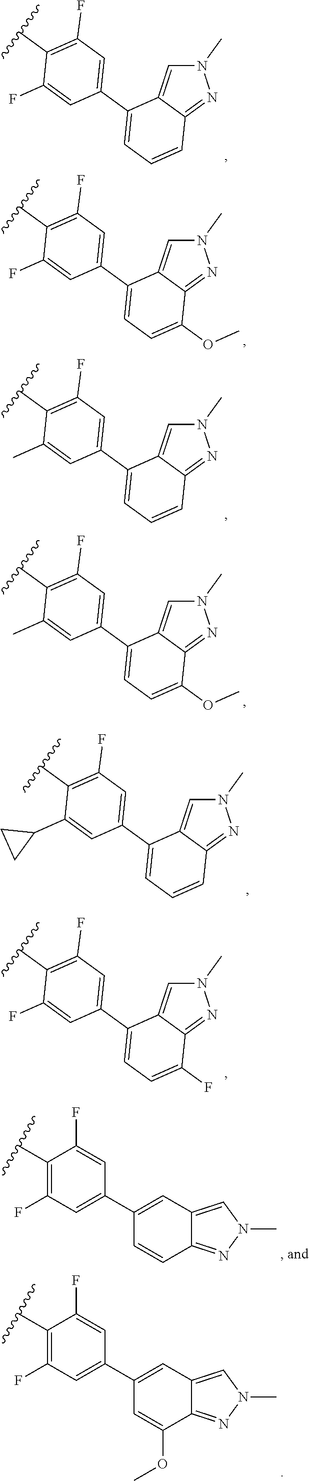

21. The compound of claim 20, or a pharmaceutically acceptable salt thereof, wherein A¹ is

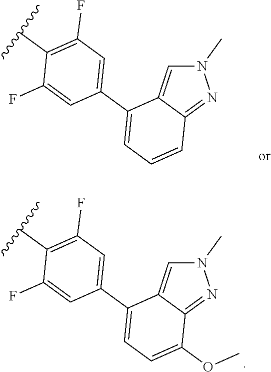 [SMILES: *c1c(F)cc(-c2cccc3nn(C)cc23)cc1F ; *c1c(F)cc(-c2ccc(OC)c3nn(C)cc23)cc1F]

22. The compound of any of claims 1-4, or a pharmaceutically acceptable salt thereof, wherein the ring system of R³ is a partially unsaturated 9-membered fused bicyclic heterocyclyl containing 1-3 nitrogen atoms, the heterocyclyl being unsubstituted or substituted as defined for R³ in claim 1.

23. The compound of any of claims 1-22, or a pharmaceutically acceptable salt thereof, wherein Y¹ is H.

24. The compound of any of claims 1-22, or a pharmaceutically acceptable salt thereof, wherein Y¹ is halogen.

25. The compound of any of claims 1-22, or a pharmaceutically acceptable salt thereof, wherein Y¹ is OH.

26. The compound of any of claims 1-22, or a pharmaceutically acceptable salt thereof, wherein Y¹ is OCD₃.

27. The compound of any of claims 1-26, or a pharmaceutically acceptable salt thereof, wherein Z¹ is N.

28. The compound of any of claims 1-26, or a pharmaceutically acceptable salt thereof, wherein Z¹ is CH.

29. The compound of any of claims 1-28, or a pharmaceutically acceptable salt thereof, wherein at least one of R³ and Y¹ comprises a deuterium label.

30. The compound of claim 1, selected from the group consisting of
6-(2,6-difluoro-4-(2-methyl-2H-indazol-4-yl)benzyl)-6,7-dihydro-5H-pyrrolo[3,4-b]pyridin-5-one-7,7-d₂;
6-(2,6-difluoro-4-(2-methyl-2H-pyrazolo[3,4-b]pyridin-4-yl)benzyl)-6,7-dihydro-5H-pyrrolo[3,4-b]pyridin-5-one-7,7-d₂;
6-(2,6-difluoro-4-(2-methyl-2H-pyrazolo[3,4-b]pyridin-5-yl)benzyl)-6,7-dihydro-5H-pyrrolo[3,4-b]pyridin-5-one-7,7-d₂;
6-(2,6-difluoro-4-(6-fluoro-2-methyl-2H-indazol-5-yl)benzyl)-6,7-dihydro-5H-pyrrolo[3,4-b]pyridin-5-one-7,7-d₂;
6-(2,6-difluoro-4-(imidazo[1,2-a]pyridin-7-yl)benzyl)-6,7-dihydro-5H-pyrrolo[3,4-b]pyridin-5-one-7,7-d₂;
6-(4-(2,6-dimethyl-2H-indazol-5-yl)-2,6-difluorobenzyl)-6,7-dihydro-5H-pyrrolo[3,4-b]pyridin-5-one-7,7-d₂;
6-(2,6-difluoro-4-(7-fluoro-2-methyl-2H-indazol-5-yl)benzyl)-6,7-dihydro-5H-pyrrolo[3,4-b]pyridin-5-one-7,7-d₂;
6-(2,6-difluoro-4-(2-methyl-2H-indazol-5-yl)benzyl)-6,7-dihydro-5H-pyrrolo[3,4-b]pyridin-5-one-7,7-d₂;
6-(2-fluoro-4-(2-methyl-2H-indazol-5-yl)benzyl)-6,7-dihydro-5H-pyrrolo[3,4-b]pyridin-5-one-7,7-d₂;
6-(2-fluoro-4-(1-methyl-1H-indazol-5-yl)benzyl)-6,7-dihydro-5H-pyrrolo[3,4-b]pyridin-5-one-7,7-d₂;
6-(4-(2-methyl-2H-indazol-4-yl)benzyl)-6,7-dihydro-5H-pyrrolo[3,4-b]pyridin-5-one-7,7-d₂;
6-(4-(2-methyl-2H-indazol-5-yl)benzyl)-6,7-dihydro-5H-pyrrolo[3,4-b]pyridin-5-one-7,7-d₂;
6-(4-(2-methyl-2H-pyrazolo[3,4-b]pyridin-5-yl)benzyl)-6,7-dihydro-5H-pyrrolo[3,4-b]pyridin-5-one-7,7-d₂;
6-(4-(2-methyl-2H-indazol-6-yl)benzyl)-6,7-dihydro-5H-pyrrolo[3,4-b]pyridin-5-one-7,7-d₂;
6-(4-(6-fluoro-2-methyl-2H-indazol-5-yl)benzyl)-6,7-dihydro-5H-pyrrolo[3,4-b]pyridin-5-one-7,7-d₂;
6-(4-(2,6-dimethyl-2H-indazol-5-yl)benzyl)-6,7-dihydro-5H-pyrrolo[3,4-b]pyridin-5-one-7,7-d₂;
6-(4-(2,7-dimethyl-2H-indazol-5-yl)benzyl)-6,7-dihydro-5H-pyrrolo[3,4-b]pyridin-5-one-7,7-d₂;
6-(4-(imidazo[1,5-a]pyridin-6-yl)benzyl)-6,7-dihydro-5H-pyrrolo[3,4-b]pyridin-5-one-7,7-d₂;
6-(4-(imidazo[1,2-a]pyridin-7-yl)benzyl)-6,7-dihydro-5H-pyrrolo[3,4-b]pyridin-5-one-7,7-d₂;
6-(2-fluoro-4-(2-methyl-2H-indazol-6-yl)benzyl)-6,7-dihydro-5H-pyrrolo[3,4-b]pyridin-5-one-7,7-d₂;
6-(2-fluoro-4-(2-methyl-2H-pyrazolo[3,4-b]pyridin-5-yl)benzyl)-6,7-dihydro-5H-pyrrolo[3,4-b]pyridin-5-one-7,7-d₂;
6-(2-fluoro-4-(2-methyl-2H-pyrazolo[3,4-b]pyridin-4-yl)benzyl)-6,7-dihydro-5H-pyrrolo[3,4-b]pyridin-5-one-7,7-d₂;
6-(2-fluoro-4-(2-methyl-2H-indazol-4-yl)benzyl)-6,7-dihydro-5H-pyrrolo[3,4-b]pyridin-5-one-7,7-d₂;
6-(2-fluoro-4-(6-fluoro-2-methyl-2H-indazol-5-yl)benzyl)-6,7-dihydro-5H-pyrrolo[3,4-b]pyridin-5-one-7,7-d₂;
6-(4-(2,7-dimethyl-2H-indazol-5-yl)-2-fluorobenzyl)-6,7-dihydro-5H-pyrrolo[3,4-b]pyridin-5-one-7,7-d₂;
6-(4-(2,6-dimethyl-2H-indazol-5-yl)-2-fluorobenzyl)-6,7-dihydro-5H-pyrrolo[3,4-b]pyridin-5-one-7,7-d₂;
6-(4-(2,7-dimethyl-2H-indazol-4-yl)-2,6-difluorobenzyl)-6,7-dihydro-5H-pyrrolo[3,4-b]pyridin-5-one-7,7-d₂;
6-(4-(2,7-dimethyl-2H-indazol-4-yl)-2-fluorobenzyl)-6,7-dihydro-5H-pyrrolo[3,4-b]pyridin-5-one-7,7-d₂;
6-(2-methyl-4-(2-methyl-2H-indazol-4-yl)benzyl)-6,7-dihydro-5H-pyrrolo[3,4-b]pyridin-5-one-7,7-d₂;
6-(4-(2,7-dimethyl-2H-indazol-4-yl)-2-methylbenzyl)-6,7-dihydro-5H-pyrrolo[3,4-b]pyridin-5-one-7,7-d₂;
6-(2,6-difluoro-4-(2-(methyl-d₃)-2H-indazol-4-yl)benzyl)-6,7-dihydro-5H-pyrrolo[3,4-b]pyridin-5-one-7,7-d₂;
6-(4-(2,6-dimethyl-2H-indazol-4-yl)-2,6-difluorobenzyl)-6,7-dihydro-5H-pyrrolo[3,4-b]pyridin-5-one-7,7-d₂;
6-(2-methyl-4-(2-methyl-2H-indazol-5-yl)benzyl)-6,7-dihydro-5H-pyrrolo[3,4-b]pyridin-5-one-7,7-d₂;
6-(2-methyl-4-(2-methyl-2H-pyrazolo[3,4-b]pyridin-5-yl)benzyl)-6,7-dihydro-5H-pyrrolo[3,4-b]pyridin-5-one-7,7-d₂;
6-(2,6-difluoro-4-(1-methyl-1H-indazol-4-yl)benzyl)-6,7-dihydro-5H-pyrrolo[3,4-b]pyridin-5-one-7,7-d₂;
6-(2,6-difluoro-4-(7-fluoro-2-methyl-2H-indazol-4-yl)benzyl)-6,7-dihydro-5H-pyrrolo[3,4-b]pyridin-5-one-7,7-d₂;
6-(2,6-difluoro-4-(5-fluoro-2-methyl-2H-indazol-4-yl)benzyl)-6,7-dihydro-5H-pyrrolo[3,4-b]pyridin-5-one-7,7-d₂;
6-(2,6-difluoro-4-(7-methoxy-2H-indazol-4-yl)benzyl)-6,7-dihydro-5H-pyrrolo[3,4-b]pyridin-5-one-7,7-d₂;
6-(2,6-difluoro-4-(7-methoxy-2-methyl-2H-indazol-4-yl)benzyl)-6,7-dihydro-5H-pyrrolo[3,4-b]pyridin-5-one-7,7-d₂;
6-(4-(7-chloro-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-methyl-7-(trifluoromethyl)-2H-indazol-4-yl)benzyl)-6,7-dihydro-5H-pyrrolo[3,4-b]pyridin-5-one-7,7-d₂;
6-(4-(2-ethyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-isopropyl-2H-indazol-4-yl)benzyl)-6,7-dihydro-5H-pyrrolo[3,4-b]pyridin-5-one-7,7-d₂;
6-(2,6-difluoro-4-(2-(fluoromethyl)-2H-indazol-4-yl)benzyl)-6,7-dihydro-5H-pyrrolo[3,4-b]pyridin-5-one-7,7-d₂;
6-(2,6-difluoro-4-(7-fluoro-2-(methyl-d₃)-2H-indazol-4-yl)benzyl)-6,7-dihydro-5H-pyrrolo[3,4-b]pyridin-5-one-7,7-d₂;
6-(2-methyl-4-(2-(methyl-d₃)-2H-indazol-4-yl)benzyl)-6,7-dihydro-5H-pyrrolo[3,4-b]pyridin-5-one-7,7-d₂;
6-(4-(7-fluoro-2-(methyl-d₃)-2H-indazol-4-yl)-2-methylbenzyl)-6,7-dihydro-5H-pyrrolo[3,4-b]pyridin-5-one-7,7-d₂;
6-(4-(7-fluoro-2-methyl-2H-indazol-4-yl)-2-methylbenzyl)-6,7-dihydro-5H-pyrrolo[3,4-b]pyridin-5-one-7,7-d₂;
6-(2,6-difluoro-4-(2-(oxetan-3-yl)-2H-indazol-4-yl)benzyl)-6,7-dihydro-5H-pyrrolo[3,4-b]pyridin-5-one-7,7-d₂;
6-(4-(2-(difluoromethyl)-2H-indazol-4-yl)-2,6-difluorobenzyl)-6,7-dihydro-5H-pyrrolo[3,4-b]pyridin-5-one-7,7-d₂;
6-(2,6-difluoro-4-(7-methoxy-2-(methyl-d₃)-2H-indazol-4-yl)benzyl)-6,7-dihydro-5H-pyrrolo[3,4-b]pyridin-5-one-7,7-d₂;
6-(2,6-difluoro-4-(7-methyl-2-(methyl-d₃)-2H-indazol-4-yl)benzyl)-6,7-dihydro-5H-pyrrolo[3,4-b]pyridin-5-one-7,7-d₂;
6-(2-fluoro-4-(2-(methyl-d₃)-2H-indazol-4-yl)benzyl)-6,7-dihydro-5H-pyrrolo[3,4-b]pyridin-5-one-7,7-d₂;
6-(2-fluoro-4-(7-fluoro-2-(methyl-d₃)-2H-indazol-4-yl)benzyl)-6,7-dihydro-5H-pyrrolo[3,4-b]pyridin-5-one-7,7-d₂;
6-(2-fluoro-4-(7-methoxy-2-(methyl-d₃)-2H-indazol-4-yl)benzyl)-6,7-dihydro-5H-pyrrolo[3,4-b]pyridin-5-one-7,7-d₂;
6-(2-fluoro-4-(7-methyl-2-(methyl-d₃)-2H-indazol-4-yl)benzyl)-6,7-dihydro-5H-pyrrolo[3,4-b]pyridin-5-one-7,7-d₂;
6-(4-(7-methoxy-2-(methyl-d₃)-2H-indazol-4-yl)-2-methylbenzyl)-6,7-dihydro-5H-pyrrolo[3,4-b]pyridin-5-one-7,7-d₂;
6-(2-methyl-4-(7-methyl-2-(methyl-d₃)-2H-indazol-4-yl)benzyl)-6,7-dihydro-5H-pyrrolo[3,4-b]pyridin-5-one-7,7-d₂;
6-(2,6-difluoro-4-(6-fluoro-2-methyl-2H-indazol-4-yl)benzyl)-6,7-dihydro-5H-pyrrolo[3,4-b]pyridin-5-one-7,7-d₂;
6-(4-(1-(difluoromethyl)-1H-indazol-4-yl)-2,6-difluorobenzyl)-6,7-dihydro-5H-pyrrolo[3,4-b]pyridin-5-one-7,7-d₂;
3-bromo-6-(2,6-difluoro-4-(2-methyl-2H-indazol-4-yl)benzyl)-6,7-dihydro-5H-pyrrolo[3,4-b]pyridin-5-one-7,7-d₂;
6-(2,6-difluoro-4-(2-methyl-2H-indazol-4-yl)benzyl)-3-hydroxy-6,7-dihydro-5H-pyrrolo[3,4-b]pyridin-5-one-7,7-d₂;
6-(2,6-difluoro-4-(2-methyl-2H-indazol-4-yl)benzyl)-3-(methoxy-d₃)-6,7-dihydro-5H-pyrrolo[3,4-b]pyridin-5-one-7,7-d₂;
6-(2,6-difluoro-4-(1-isopropyl-1H-indazol-4-yl)benzyl)-6,7-dihydro-5H-pyrrolo[3,4-b]pyridin-5-one-7,7-d₂;
6-(4-(1-ethyl-1H-indazol-4-yl)-2,6-difluorobenzyl)-6,7-dihydro-5H-pyrrolo[3,4-b]pyridin-5-one-7,7-d₂;
6-(2,6-difluoro-4-(1-(oxetan-3-yl)-1H-indazol-4-yl)benzyl)-6,7-dihydro-5H-pyrrolo[3,4-b]pyridin-5-one-7,7-d₂;
6-(2,6-difluoro-4-(1-(methyl-d₃)-1H-indazol-4-yl)benzyl)-6,7-dihydro-5H-pyrrolo[3,4-b]pyridin-5-one-7,7-d₂;
6-(2,6-difluoro-4-(7-fluoro-1-(methyl-d₃)-1H-indazol-4-yl)benzyl)-6,7-dihydro-5H-pyrrolo[3,4-b]pyridin-5-one-7,7-d₂;
6-(2,6-difluoro-4-(7-methoxy-1-(methyl-d₃)-1H-indazol-4-yl)benzyl)-6,7-dihydro-5H-pyrrolo[3,4-b]pyridin-5-one-7,7-d₂;
6-(2,6-difluoro-4-(7-methyl-1-(methyl-d₃)-1H-indazol-4-yl)benzyl)-6,7-dihydro-5H-pyrrolo[3,4-b]pyridin-5-one-7,7-d₂;
6-(2-fluoro-4-(6-fluoro-2-methyl-2H-indazol-4-yl)benzyl)-6,7-dihydro-5H-pyrrolo[3,4-b]pyridin-5-one-7,7-d₂;
6-(4-(6-fluoro-2-methyl-2H-indazol-4-yl)benzyl)-6,7-dihydro-5H-pyrrolo[3,4-b]pyridin-5-one-7,7-d₂;
6-(4-(7-chloro-2-(methyl-d₃)-2H-indazol-4-yl)-2,6-difluorobenzyl)-6,7-dihydro-5H-pyrrolo[3,4-b]pyridin-5-one-7,7-d₂;
6-(2,6-difluoro-4-(2-(methyl-d₃)-7-(trifluoromethyl)-2H-indazol-4-yl)benzyl)-6,7-dihydro-5H-pyrrolo[3,4-b]pyridin-5-one-7,7-d₂;
6-(4-(6-chloro-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(7-methoxy-2-methyl-2H-indazol-5-yl)benzyl)-6,7-dihydro-5H-pyrrolo[3,4-b]pyridin-5-one-7,7-d₂;
6-(4-(7-chloro-6-fluoro-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-(methyl-d₃)-2H-indazol-4-yl)benzyl)-3-(methoxy-d₃)-6,7-dihydro-5H-pyrrolo[3,4-b]pyridin-5-one-7,7-d₂;
4-(3,5-difluoro-4-((5-oxo-5,7-dihydro-6H-pyrrolo[3,4-b]pyridin-6-yl-7,7-d₂)methyl)phenyl)-2,6-dimethyl-2,6-dihydro-7H-pyrazolo[3,4-c]pyridin-7-one;
4-(3,5-difluoro-4-((5-oxo-5,7-dihydro-6H-pyrrolo[3,4-b]pyridin-6-yl-7,7-d₂)methyl)phenyl)-1,6-dimethyl-1,6-dihydro-7H-pyrazolo[3,4-c]pyridin-7-one;
6-(4-(6-(difluoromethyl)-2-methyl-2H-indazol-4-yl)-2,6-difluorobenzyl)-6,7-dihydro-5H-pyrrolo[3,4-b]pyridin-5-one-7,7-d₂;
6-(2-fluoro-4-(2-methyl-2H-indazol-4-yl)benzyl)-3-(methoxy-d₃)-6,7-dihydro-5H-pyrrolo[3,4-b]pyridin-5-one-7,7-d₂;
3-(methoxy-d₃)-6-(4-(2-methyl-2H-indazol-4-yl)benzyl)-6,7-dihydro-5H-pyrrolo[3,4-b]pyridin-5-one-7,7-d₂;
6-(4-(6-fluoro-2-methyl-2H-indazol-4-yl)-2-methylbenzyl)-6,7-dihydro-5H-pyrrolo[3,4-b]pyridin-5-one-7,7-d₂;
6-(4-(6-chloro-2-(methyl-d₃)-2H-indazol-4-yl)-2,6-difluorobenzyl)-6,7-dihydro-5H-pyrrolo[3,4-b]pyridin-5-one-7,7-d₂;
6-(2,6-difluoro-4-(6-fluoro-2-(methyl-d₃)-2H-indazol-4-yl)benzyl)-6,7-dihydro-5H-pyrrolo[3,4-b]pyridin-5-one-7,7-d₂;
6-(2,6-difluoro-4-(6-(methoxy-d₃)-2-(methyl-d₃)-2H-indazol-4-yl)benzyl)-6,7-dihydro-5H-pyrrolo[3,4-b]pyridin-5-one-7,7-d₂;
6-(2,6-difluoro-4-(6-(methoxy-d₃)-1-(methyl-d₃)-1H-indazol-4-yl)benzyl)-6,7-dihydro-5H-pyrrolo[3,4-b]pyridin-5-one-7,7-d₂;
6-(4-(7-chloro-2-methyl-2H-indazol-5-yl)-2,6-difluorobenzyl)-6,7-dihydro-5H-pyrrolo[3,4-b]pyridin-5-one-7,7-d₂;
6-(2,6-difluoro-4-(6-methoxy-2-methyl-2H-indazol-4-yl)benzyl)-6,7-dihydro-5H-pyrrolo[3,4-b]pyridin-5-one-7,7-d₂;
4-(3,5-difluoro-4-((5-oxo-5,7-dihydro-6H-pyrrolo[3,4-b]pyridin-6-yl-7,7-d₂)methyl)phenyl)-6-methyl-2-(methyl-d₃)-2,6-dihydro-7H-pyrazolo[3,4-c]pyridin-7-one;
4-(3,5-difluoro-4-((5-oxo-5,7-dihydro-6H-pyrrolo[3,4-b]pyridin-6-yl-7,7-d₂)methyl)phenyl)-6-methyl-1-(methyl-d₃)-1,6-dihydro-7H-pyrazolo[3,4-c]pyridin-7-one;
6-(2,6-difluoro-4-(7-(methoxy-d₃)-2-methyl-2H-indazol-4-yl)benzyl)-6,7-dihydro-5H-pyrrolo[3,4-b]pyridin-5-one-7,7-d₂;
6-(2,6-difluoro-4-(7-(methoxy-d₃)-2-(methyl-d₃)-2H-indazol-4-yl)benzyl)-6,7-dihydro-5H-pyrrolo[3,4-b]pyridin-5-one-7,7-d₂;
6-(2,6-difluoro-4-(7-(methoxy-d₃)-1-(methyl-d₃)-1H-indazol-4-yl)benzyl)-6,7-dihydro-5H-pyrrolo[3,4-b]pyridin-5-one-7,7-d₂;
6-(4-(7-ethoxy-2-methyl-2H-indazol-4-yl)-2,6-difluorobenzyl)-6,7-dihydro-5H-pyrrolo[3,4-b]pyridin-5-one-7,7-d₂;
6-(4-(7-ethoxy-2-(methyl-d₃)-2H-indazol-4-yl)-2,6-difluorobenzyl)-6,7-dihydro-5H-pyrrolo[3,4-b]pyridin-5-one-7,7-d₂;
6-(2,6-difluoro-4-(7-isopropoxy-2-methyl-2H-indazol-4-yl)benzyl)-6,7-dihydro-5H-pyrrolo[3,4-b]pyridin-5-one-7,7-d₂;
6-(2,6-difluoro-4-(7-isopropoxy-2-(methyl-d₃)-2H-indazol-4-yl)benzyl)-6,7-dihydro-5H-pyrrolo[3,4-b]pyridin-5-one-7,7-d₂;
6-(2,6-difluoro-4-(2-(methyl-d₃)-2H-indazol-5-yl)benzyl)-6,7-dihydro-5H-pyrrolo[3,4-b]pyridin-5-one-7,7-d₂;
6-(2,6-difluoro-4-(1-(methyl-d₃)-1H-indazol-5-yl)benzyl)-6,7-dihydro-5H-pyrrolo[3,4-b]pyridin-5-one-7,7-d₂;
6-(4-(7-(difluoromethoxy)-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-methyl-7-(trifluoromethoxy)-2H-indazol-4-yl)benzyl)-6,7-dihydro-5H-pyrrolo[3,4-b]pyridin-5-one-7,7-d₂;
6-(2,6-difluoro-4-(2-(methyl-d₃)-7-(trifluoromethoxy)-2H-indazol-4-yl)benzyl)-6,7-dihydro-5H-pyrrolo[3,4-b]pyridin-5-one-7,7-d₂;
6-(4-(6,7-difluoro-2-methyl-2H-indazol-4-yl)-2,6-difluorobenzyl)-6,7-dihydro-5H-pyrrolo[3,4-b]pyridin-5-one-7,7-d₂;
6-(4-(6,7-difluoro-2-(methyl-d₃)-2H-indazol-4-yl)-2,6-difluorobenzyl)-6,7-dihydro-5H-pyrrolo[3,4-b]pyridin-5-one-7,7-d₂;
6-(2-fluoro-4-(7-(methoxy-d₃)-2-methyl-2H-indazol-4-yl)benzyl)-6,7-dihydro-5H-pyrrolo[3,4-b]pyridin-5-one-7,7-d₂;
6-(4-(7-ethoxy-2-methyl-2H-indazol-4-yl)-2-fluorobenzyl)-6,7-dihydro-5H-pyrrolo[3,4-b]pyridin-5-one-7,7-d₂;
6-(2-fluoro-4-(7-isopropoxy-2-methyl-2H-indazol-4-yl)benzyl)-6,7-dihydro-5H-pyrrolo[3,4-b]pyridin-5-one-7,7-d₂;
6-(2-fluoro-4-(2-methyl-7-(trifluoromethoxy)-2H-indazol-4-yl)benzyl)-6,7-dihydro-5H-pyrrolo[3,4-b]pyridin-5-one-7,7-d₂;
6-(2-fluoro-4-(2-methyl-2H-indazol-5-yl)benzyl)-3-(methoxy-d₃)-6,7-dihydro-5H-pyrrolo[3,4-b]pyridin-5-one-7,7-d₂;
6-(2,6-difluoro-4-(2-methyl-2H-indazol-5-yl)benzyl)-3-(methoxy-d₃)-6,7-dihydro-5H-pyrrolo[3,4-b]pyridin-5-one-7,7-d₂;
6-(2,6-difluoro-4-(7-(2-methoxyethoxy)-2-methyl-2H-indazol-4-yl)benzyl)-6,7-dihydro-5H-pyrrolo[3,4-b]pyridin-5-one-7,7-d₂;
6-(2,6-difluoro-4-(7-(2-methoxyethoxy)-2-(methyl-d₃)-2H-indazol-4-yl)benzyl)-6,7-dihydro-5H-pyrrolo[3,4-b]pyridin-5-one-7,7-d₂;
6-(2,6-difluoro-4-(2-(methyl-d₃)-2H-pyrazolo[3,4-b]pyridin-5-yl)benzyl)-6,7-dihydro-5H-pyrrolo[3,4-b]pyridin-5-one-7,7-d₂;
6-(2-fluoro-4-(7-(2-methoxyethoxy)-2-methyl-2H-indazol-4-yl)benzyl)-6,7-dihydro-5H-pyrrolo[3,4-b]pyridin-5-one-7,7-d₂;
6-(2-fluoro-4-(7-(2-methoxyethoxy)-2-(methyl-d₃)-2H-indazol-4-yl)benzyl)-6,7-dihydro-5H-pyrrolo[3,4-b]pyridin-5-one-7,7-d₂;
6-(2-fluoro-4-(2-(methyl-d₃)-2H-pyrazolo[3,4-b]pyridin-5-yl)benzyl)-6,7-dihydro-5H-pyrrolo[3,4-b]pyridin-5-one-7,7-d₂;
6-(2,6-difluoro-4-(2-methyl-2H-pyrazolo[3,4-c]pyridin-4-yl)benzyl)-6,7-dihydro-5H-pyrrolo[3,4-b]pyridin-5-one-7,7-d₂;
6-(2-fluoro-4-(2-methyl-2H-pyrazolo[3,4-c]pyridin-4-yl)benzyl)-6,7-dihydro-5H-pyrrolo[3,4-b]pyridin-5-one-7,7-d₂;
6-(2,6-difluoro-4-(7-methoxy-2-methyl-2H-pyrazolo[3,4-c]pyridin-4-yl)benzyl)-6,7-dihydro-5H-pyrrolo[3,4-b]pyridin-5-one-7,7-d₂;
6-(4-(7-cyclopropyl-2-methyl-2H-indazol-4-yl)-2,6-difluorobenzyl)-6,7-dihydro-5H-pyrrolo[3,4-b]pyridin-5-one-7,7-d₂;
6-(4-(7-(difluoromethyl)-2-methyl-2H-indazol-4-yl)-2,6-difluorobenzyl)-6,7-dihydro-5H-pyrrolo[3,4-b]pyridin-5-one-7,7-d₂;
6-(4-(7-cyclopropoxy-2-methyl-2H-indazol-4-yl)-2,6-difluorobenzyl)-6,7-dihydro-5H-pyrrolo[3,4-b]pyridin-5-one-7,7-d₂;
6-(4-(7-cyclobutoxy-2-methyl-2H-indazol-4-yl)-2,6-difluorobenzyl)-6,7-dihydro-5H-pyrrolo[3,4-b]pyridin-5-one-7,7-d₂
6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂;
6-(2,6-difluoro-4-(2-(methyl-d₃)-7-((tetrahydrofuran-3-yl)oxy)-2H-indazol-4-yl)benzyl)-6,7-dihydro-5H-pyrrolo[3,4-b]pyridin-5-one-7,7-d₂;
6-(4-(7-(benzyloxy)-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(7-hydroxy-2-methyl-2H-indazol-4-yl)benzyl)-6,7-dihydro-5H-pyrrolo[3,4-b]pyridin-5-one-7,7-d₂;
6-(4-(7-(2-(dimethylamino)ethoxy)-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(7-hydroxy-2-(methyl-d₃)-2H-indazol-4-yl)benzyl)-6,7-dihydro-5H-pyrrolo[3,4-b]pyridin-5-one-7,7-d₂;
6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂;
6-(2,6-difluoro-4-(2-methyl-7-((tetrahydrofuran-2-yl)methoxy)-2H-indazol-4-yl)benzyl)-6,7-dihydro-5H-pyrrolo[3,4-b]pyridin-5-one-7,7-d₂;
6-(2,6-difluoro-4-(2-methyl-7-(oxetan-2-ylmethoxy)-2H-indazol-4-yl)benzyl)-6,7-dihydro-5H-pyrrolo[3,4-b]pyridin-5-one-7,7-d₂;
(S)-6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂;
(R)-6-(2,6-difluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂;
6-(4-(7-(cyclopropylmethoxy)-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-methyl-7-(oxetan-3-yloxy)-2H-indazol-4-yl)benzyl)-6,7-dihydro-5H-pyrrolo[3,4-b]pyridin-5-one-7,7-d₂;
6-(2,6-difluoro-4-(2-methyl-7-((tetrahydro-2H-pyran-4-yl)oxy)-2H-indazol-4-yl)benzyl)-6,7-dihydro-5H-pyrrolo[3,4-b]pyridin-5-one-7,7-d₂;
6-(2,6-difluoro-4-(2-(methyl-d₃)-2H-indazol-4-yl)benzyl)-3-hydroxy-6,7-dihydro-5H-pyrrolo[3,4-b]pyridin-5-one-7,7-d₂;
6-(2-fluoro-4-(7-methoxy-2-methyl-2H-indazol-4-yl)benzyl)-3-(methoxy-d₃)-6,7-dihydro-5H-pyrrolo[3,4-b]pyridin-5-one-7,7-d₂;
6-(2-fluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂;
(S)-6-(2-fluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂;
6-(2-fluoro-4-(2-methyl-7-(oxetan-2-ylmethoxy)-2H-indazol-4-yl)benzyl)-6,7-dihydro-5H-pyrrolo[3,4-b]pyridin-5-one-7,7-d₂;
6-(2,6-difluoro-4-(7-methoxy-2-methyl-2H-indazol-4-yl)benzyl)-3-(methoxy-d₃)-6,7-dihydro-5H-pyrrolo[3,4-b]pyridin-5-one-7,7-d₂;
6-(2-fluoro-4-(2-methyl-7-((tetrahydrofuran-2-yl)methoxy)-2H-indazol-4-yl)benzyl)-6,7-dihydro-5H-pyrrolo[3,4-b]pyridin-5-one-7,7-d₂;
(R)-6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂;
6-(2,6-difluoro-4-(7-(2-isopropoxyethoxy)-2-methyl-2H-indazol-4-yl)benzyl)-6,7-dihydro-5H-pyrrolo[3,4-b]pyridin-5-one-7,7-d₂;
6-(4-(7-(2-cyclopropoxyethoxy)-2-methyl-2H-indazol-4-yl)-2,6-difluorobenzyl)-6,7-dihydro-5H-pyrrolo[3,4-b]pyridin-5-one-7,7-d₂;
(R)-6-(2-fluoro-4-(2-methyl-7-((tetrahydrofuran-3-yl)oxy)-2H-indazol-4-yl)benzyl)-6,7-dihydro-5H-pyrrolo[3,4-b]pyridin-5-one-7,7-d₂;
(S)-6-(2-fluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂;
6-(2-fluoro-4-(7-(2-isopropoxyethoxy)-2-methyl-2H-indazol-4-yl)benzyl)-6,7-dihydro-5H-pyrrolo[3,4-b]pyridin-5-one-7,7-d₂;
6-(4-(7-(2-cyclopropoxyethoxy)-2-methyl-2H-indazol-4-yl)-2-fluorobenzyl)-6,7-dihydro-5H-pyrrolo[3,4-b]pyridin-5-one-7,7-d₂;
6-(2-fluoro-6-methyl-4-(2-methyl-2H-indazol-4-yl)benzyl)-6,7-dihydro-5H-pyrrolo[3,4-b]pyridin-5-one-7,7-d₂;
6-(2-fluoro-6-methyl-4-(2-(methyl-d₃)-2H-indazol-4-yl)benzyl)-6,7-dihydro-5H-pyrrolo[3,4-b]pyridin-5-one-7,7-d₂;
6-(2-fluoro-6-methyl-4-(7-methyl-2-(methyl-d₃)-2H-indazol-4-yl)benzyl)-6,7-dihydro-5H-pyrrolo[3,4-b]pyridin-5-one-7,7-d₂;
6-(2-fluoro-4-(7-fluoro-2-(methyl-d₃)-2H-indazol-4-yl)-6-methylbenzyl)-6,7-dihydro-5H-pyrrolo[3,4-b]pyridin-5-one-7,7-d₂;
6-(2-fluoro-4-(7-methoxy-2-(methyl-d₃)-2H-indazol-4-yl)-6-methylbenzyl)-6,7-dihydro-5H-pyrrolo[3,4-b]pyridin-5-one-7,7-d₂;
6-(2-fluoro-6-methyl-4-(2-(methyl-d₃)-2H-indazol-5-yl)benzyl)-6,7-dihydro-5H-pyrrolo[3,4-b]pyridin-5-one-7,7-d₂;
6-(2-chloro-6-fluoro-4-(2-(methyl-d₃)-2H-indazol-4-yl)benzyl)-6,7-dihydro-5H-pyrrolo[3,4-b]pyridin-5-one-7,7-d₂;
6-(2-chloro-4-(2,7-dimethyl-2H-indazol-4-yl)-6-fluorobenzyl)-6,7-dihydro-5H-pyrrolo[3,4-b]pyridin-5-one-7,7-d₂;
6-(2-chloro-6-fluoro-4-(7-methoxy-2-methyl-2H-indazol-4-yl)benzyl)-6,7-dihydro-5H-pyrrolo[3,4-b]pyridin-5-one-7,7-d₂;
6-(2-chloro-6-fluoro-4-(7-fluoro-2-methyl-2H-indazol-4-yl)benzyl)-6,7-dihydro-5H-pyrrolo[3,4-b]pyridin-5-one-7,7-d₂;
6-(2-chloro-6-fluoro-4-(2-methyl-2H-pyrazolo[3,4-b]pyridin-4-yl)benzyl)-6,7-dihydro-5H-pyrrolo[3,4-b]pyridin-5-one-7,7-d₂;
6-(2-chloro-6-fluoro-4-(2-methyl-2H-indazol-4-yl)benzyl)-6,7-dihydro-5H-pyrrolo[3,4-b]pyridin-5-one-7,7-d₂;
6-(2-chloro-6-fluoro-4-(2-methyl-2H-indazol-5-yl)benzyl)-6,7-dihydro-5H-pyrrolo[3,4-b]pyridin-5-one-7,7-d₂;
6-(2-chloro-6-fluoro-4-(2-methyl-2H-pyrazolo[3,4-b]pyridin-5-yl)benzyl)-6,7-dihydro-5H-pyrrolo[3,4-b]pyridin-5-one-7,7-d₂;
6-(2-chloro-4-(7-cyclopropoxy-2-methyl-2H-indazol-4-yl)-6-fluorobenzyl)-6,7-dihydro-5H-pyrrolo[3,4-b]pyridin-5-one-7,7-d₂;
6-(2-chloro-6-fluoro-4-(7-(2-methoxyethoxy)-2-methyl-2H-indazol-4-yl)benzyl)-6,7-dihydro-5H-pyrrolo[3,4-b]pyridin-5-one-7,7-d₂;
(S)-6-(2,6-difluoro-4-(7-((1-methoxypropan-2-yl)oxy)-2-methyl-2H-indazol-4-yl)benzyl)-6,7-dihydro-5H-pyrrolo[3,4-b]pyridin-5-one-7,7-d₂;
(R)-6-(2,6-difluoro-4-(7-(2-methoxypropoxy)-2-methyl-2H-indazol-4-yl)benzyl)-6,7-dihydro-5H-pyrrolo[3,4-b]pyridin-5-one-7,7-d₂;
tert-butyl 3-((4-(3,5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)azetidine-1-carboxylate;
1-(((4-(3, 5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)cyclopropane-1-carbonitrile;
(R)-6-(2-fluoro-4-(7-(2-methoxypropoxy)-2-methyl-2H-indazol-4-yl)benzyl)-6,7-dihydro-5H-pyrrolo[3,4-b]pyridin-5-one-7,7-d₂;
tert-butyl 3-((4-(3-fluoro-4-((5-oxo-5,7-dihydro-6H-pyrrolo[3,4-b]pyridin-6-yl-7,7-d₂)methyl)phenyl)-2-methyl-2H-indazol-7-yl)oxy)azetidine-1-carboxylate;
6-(2-fluoro-4-(7-(2-methoxyethoxy)-2-methyl-2H-indazol-4-yl)-6-methylbenzyl)-6,7-dihydro-5H-pyrrolo[3,4-b]pyridin-5-one-7,7-d₂;
1-(((4-(3-fluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)cyclopropane-1-carbonitrile;
tert-butyl 3-(((4-(3, 5-difluoro-4-((5-oxo-5,7-dihydro-6H-pyrrolo[3,4-b]pyridin-6-yl-7,7-d₂)methyl)phenyl)-2-methyl-2H-indazol-7-yl)oxy)methyl)azetidine-1-carboxylate;
6-(2,6-difluoro-4-(7-((1-isopropylazetidin-3-yl)oxy)-2-methyl-2H-indazol-4-yl)benzyl)-6,7-dihydro-5H-pyrrolo[3,4-b]pyridin-5-one-7,7-d₂;
6-(2,6-difluoro-4-(2-methyl-7-(methylthio)-2H-indazol-4-yl)benzyl)-6,7-dihydro-5H-pyrrolo[3,4-b]pyridin-5-one-7,7-d₂;
6-(2,6-difluoro-4-(2-methyl-7-(S-methylsulfonimidoyl)-2H-indazol-4-yl)benzyl)-6,7-dihydro-5H-pyrrolo[3,4-b]pyridin-5-one-7,7-d₂;
6-(4-(7-cyclopropoxy-2-(methyl-d₃)-2H-indazol-4-yl)-2,6-difluorobenzyl)-6,7-dihydro-5H-pyrrolo[3,4-b]pyridin-5-one-7,7-d₂;
6-(4-(7-((1-butylazetidin-3-yl)oxy)-2-methyl-2H-indazol-4-yl)-2,6-difluorobenzyl)-6,7-dihydro-5H-pyrrolo[3,4-b]pyridin-5-one-7,7-d₂;
6-(2,6-difluoro-4-(2-methyl-7-((methylthio)methoxy)-2H-indazol-4-yl)benzyl)-6,7-dihydro-5H-pyrrolo[3,4-b]pyridin-5-one-7,7-d₂;
6-(2,6-difluoro-4-(2-methyl-7-((methylsulfonyl)methoxy)-2H-indazol-4-yl)benzyl)-6,7-dihydro-5H-pyrrolo[3,4-b]pyridin-5-one-7,7-d₂;
6-(2,6-difluoro-4-(7-((1-(isopropylsulfonyl)azetidin-3-yl)oxy)-2-methyl-2H-indazol-4-yl)benzyl)-6,7-dihydro-5H-pyrrolo[3,4-b]pyridin-5-one-7,7-d₂;
6-(2,6-difluoro-4-(2-(methyl-d₃)-2H-pyrazolo[3,4-b]pyridin-4-yl)benzyl)-6,7-dihydro-5H-pyrrolo[3,4-b]pyridin-5-one-7,7-d₂;
6-(4-(7-((1-cyclohexylazetidin-3-yl)oxy)-2-methyl-2H-indazol-4-yl)-2,6-difluorobenzyl)-6,7-dihydro-5H-pyrrolo[3,4-b]pyridin-5-one-7,7-d₂;
6-(2-cyclopropyl-6-fluoro-4-(2-(methyl-d₃)-2H-indazol-4-yl)benzyl)-6,7-dihydro-5H-pyrrolo[3,4-b]pyridin-5-one-7,7-d₂;
6-(2-cyclopropyl-6-fluoro-4-(3-fluoro-2-(methyl-d₃)-2H-indazol-4-yl)benzyl)-6,7-dihydro-5H-pyrrolo[3,4-b]pyridin-5-one-7,7-d₂;
6-(2-cyclopropyl-6-fluoro-4-(2-methyl-2H-indazol-4-yl)benzyl)-6,7-dihydro-5H-pyrrolo[3,4-b]pyridin-5-one-7,7-d₂;
6-(2-cyclopropyl-6-fluoro-4-(2-methyl-2H-indazol-5-yl)benzyl)-6,7-dihydro-5H-pyrrolo[3,4-b]pyridin-5-one-7,7-d₂;
6-(2-fluoro-6-methyl-4-(2-methyl-2H-indazol-5-yl)benzyl)-6,7-dihydro-5H-pyrrolo[3,4-b]pyridin-5-one-7,7-d₂;
6-(2-fluoro-4-(7-methoxy-2-methyl-2H-indazol-4-yl)-6-methylbenzyl)-6,7-dihydro-5H-pyrrolo[3,4-b]pyridin-5-one-7,7-d₂;
6-(4-(7-cyclopropoxy-2-methyl-2H-indazol-4-yl)-2-fluoro-6-methylbenzyl)-6,7-dihydro-5H-pyrrolo[3,4-b]pyridin-5-one-7,7-d₂;
6-(4-(2,7-dimethyl-2H-indazol-4-yl)-2-fluoro-6-methylbenzyl)-6,7-dihydro-5H-pyrrolo[3,4-b]pyridin-5-one-7,7-d₂;
6-(2-fluoro-4-(7-fluoro-2-methyl-2H-indazol-4-yl)-6-methylbenzyl)-6,7-dihydro-5H-pyrrolo[3,4-b]pyridin-5-one-7,7-d₂;
6-(4-(7-chloro-2-methyl-2H-indazol-4-yl)-2-fluoro-6-methylbenzyl)-6,7-dihydro-5H-pyrrolo[3,4-b]pyridin-5-one-7,7-d₂;
6-(2-fluoro-6-methyl-4-(2-methyl-7-(trifluoromethyl)-2H-indazol-4-yl)benzyl)-6,7-dihydro-5H-pyrrolo[3,4-b]pyridin-5-one-7,7-d₂;
6-(2-fluoro-6-methyl-4-(2-methyl-2H-pyrazolo[3,4-b]pyridin-4-yl)benzyl)-6,7-dihydro-5H-pyrrolo[3,4-b]pyridin-5-one-7,7-d₂;
6-(4-(7-cyclopropoxy-2-methyl-2H-indazol-4-yl)-2-cyclopropyl-6-fluorobenzyl)-6,7-dihydro-5H-pyrrolo[3,4-b]pyridin-5-one-7,7-d₂;
6-(2-cyclopropyl-6-fluoro-4-(7-(2-methoxyethoxy)-2-methyl-2H-indazol-4-yl)benzyl)-6,7-dihydro-5H-pyrrolo[3,4-b]pyridin-5-one-7,7-d₂;
6-(2-cyclopropyl-4-(2,7-dimethyl-2H-indazol-4-yl)-6-fluorobenzyl)-6,7-dihydro-5H-pyrrolo[3,4-b]pyridin-5-one-7,7-d₂;
6-(2-cyclopropyl-6-fluoro-4-(7-fluoro-2-methyl-2H-indazol-4-yl)benzyl)-6,7-dihydro-5H-pyrrolo[3,4-b]pyridin-5-one-7,7-d₂;
6-(4-(7-chloro-2-methyl-2H-indazol-4-yl)-2-cyclopropyl-6-fluorobenzyl)-6,7-dihydro-5H-pyrrolo[3,4-b]pyridin-5-one-7,7-d₂;
6-(2-cyclopropyl-6-fluoro-4-(2-methyl-7-(trifluoromethyl)-2H-indazol-4-yl)benzyl)-6,7-dihydro-5H-pyrrolo[3,4-b]pyridin-5-one-7,7-d₂;
6-(2-cyclopropyl-6-fluoro-4-(2-methyl-2H-pyrazolo[3,4-b]pyridin-4-yl)benzyl)-6,7-dihydro-5H-pyrrolo[3,4-b]pyridin-5-one-7,7-d₂;
6-(2-cyclopropyl-6-fluoro-4-(7-methoxy-2-(methyl-d₃)-2H-indazol-4-yl)benzyl)-6,7-dihydro-5H-pyrrolo[3,4-b]pyridin-5-one-7,7-d₂;
6-(2-fluoro-6-isobutoxy-4-(2-methyl-2H-indazol-4-yl)benzyl)-6,7-dihydro-5H-pyrrolo[3,4-b]pyridin-5-one-7,7-d₂;
6-(2-fluoro-6-isobutoxy-4-(2-methyl-2H-indazol-5-yl)benzyl)-6,7-dihydro-5H-pyrrolo[3,4-b]pyridin-5-one-7,7-d₂;
6-(4-(7-cyclopropoxy-2-methyl-2H-indazol-4-yl)-2-fluoro-6-isobutoxybenzyl)-6,7-dihydro-5H-pyrrolo[3,4-b]pyridin-5-one-7,7-d₂;
6-(4-(2,7-dimethyl-2H-indazol-4-yl)-2-fluoro-6-isobutoxybenzyl)-6,7-dihydro-5H-pyrrolo[3,4-b]pyridin-5-one-7,7-d₂;
6-(2-fluoro-4-(7-fluoro-2-methyl-2H-indazol-4-yl)-6-isobutoxybenzyl)-6,7-dihydro-5H-pyrrolo[3,4-b]pyridin-5-one-7,7-d₂;
6-(4-(7-chloro-2-methyl-2H-indazol-4-yl)-2-fluoro-6-isobutoxybenzyl)-6,7-dihydro-5H-pyrrolo[3,4-b]pyridin-5-one-7,7-d₂;
6-(2-fluoro-6-isobutoxy-4-(2-methyl-7-(trifluoromethyl)-2H-indazol-4-yl)benzyl)-6,7-dihydro-5H-pyrrolo[3,4-b]pyridin-5-one-7,7-d₂;
6-(2-fluoro-6-isobutoxy-4-(2-methyl-2H-pyrazolo[3,4-b]pyridin-4-yl)benzyl)-6,7-dihydro-5H-pyrrolo[3,4-b]pyridin-5-one-7,7-d₂;
6-(2-fluoro-6-isobutoxy-4-(7-(2-methoxyethoxy)-2-methyl-2H-indazol-4-yl)benzyl)-6,7-dihydro-5H-pyrrolo[3,4-b]pyridin-5-one-7,7-d₂;
6-(2-chloro-6-cyclopropyl-4-(2-methyl-2H-indazol-4-yl)benzyl)-6,7-dihydro-5H-pyrrolo[3,4-]pyridin-5-one-7,7-d₂;
6-(2-chloro-6-cyclopropyl-4-(2-(methyl-d₃)-2H-indazol-4-yl)benzyl)-6,7-dihydro-5H-pyrrolo[3,4-b]pyridin-5-one-7,7-d₂;
6-(2-(azetidin-1-yl)-6-fluoro-4-(2-methyl-2H-indazol-4-yl)benzyl)-6,7-dihydro-5H-pyrrolo[3,4-b]pyridin-5-one-7,7-d₂;
6-(2-(azetidin-1-yl)-6-fluoro-4-(2-(methyl-d₃)-2H-indazol-4-yl)benzyl)-6,7-dihydro-5H-pyrrolo[3,4-b]pyridin-5-one-7,7-d₂;
6-(2-cyclopentyl-6-fluoro-4-(2-methyl-2H-indazol-4-yl)benzyl)-6,7-dihydro-5H-pyrrolo[3,4-b]pyridin-5-one-7,7-d₂;
6-(2-cyclopentyl-6-fluoro-4-(2-(methyl-d₃)-2H-indazol-4-yl)benzyl)-6,7-dihydro-5H-pyrrolo[3,4-b]pyridin-5-one-7,7-d₂;
6-(2-fluoro-4-(2-methyl-2H-indazol-4-yl)-6-(trifluoromethyl)benzyl)-6,7-dihydro-5H-pyrrolo[3,4-b]pyridin-5-one-7,7-d₂;
6-(2-fluoro-4-(2-(methyl-d₃)-2H-indazol-4-yl)-6-(trifluoromethyl)benzyl)-6,7-dihydro-5H-pyrrolo[3,4-b]pyridin-5-one-7,7-d₂;
6-(2,6-dichloro-4-(2-methyl-2H-indazol-4-yl)benzyl)-6,7-dihydro-5H-pyrrolo[3,4-b]pyridin-5-one-7,7-d₂;
6-(2,6-dichloro-4-(2-(methyl-d₃)-2H-indazol-4-yl)benzyl)-6,7-dihydro-5H-pyrrolo[3,4-b]pyridin-5-one-7,7-d₂;
6-(2-(dimethylamino)-6-fluoro-4-(2-methyl-2H-indazol-4-yl)benzyl)-6,7-dihydro-5H-pyrrolo[3,4-b]pyridin-5-one-7,7-d₂;
6-(2-(dimethylamino)-6-fluoro-4-(2-(methyl-d₃)-2H-indazol-4-yl)benzyl)-6,7-dihydro-5H-pyrrolo[3,4-b]pyridin-5-one-7,7-d₂;
6-(2-fluoro-6-methoxy-4-(2-methyl-2H-indazol-4-yl)benzyl)-6,7-dihydro-5H-pyrrolo[3,4-b]pyridin-5-one-7,7-d₂;
6-(2-fluoro-6-methoxy-4-(2-(methyl-d₃)-2H-indazol-4-yl)benzyl)-6,7-dihydro-5H-pyrrolo[3,4-b]pyridin-5-one-7,7-d₂;
6-(2-isopropyl-4-(2-methyl-2H-indazol-4-yl)benzyl)-6,7-dihydro-5H-pyrrolo[3,4-b]pyridin-5-one-7,7-d₂;
6-(2-isopropyl-4-(2-(methyl-d₃)-2H-indazol-4-yl)benzyl)-6,7-dihydro-5H-pyrrolo[3,4-b]pyridin-5-one-7,7-d₂;
6-(4-(1-methyl-1H-indazol-5-yl)benzyl)-6,7-dihydro-5H-pyrrolo[3,4-b]pyridin-5-one-7,7-d₂;
6-(4-(2,4-dimethyl-2H-indazol-5-yl)benzyl)-6,7-dihydro-5H-pyrrolo[3,4-b]pyridin-5-one-7,7-d₂;
6-(4-([1,2,4]triazolo[4,3-a]pyridin-5-yl)benzyl)-6,7-dihydro-5H-pyrrolo[3,4-b]pyridin-5-one-7,7-d₂;
6-(4-([1,2,4]triazolo[4,3-a]pyridin-5-yl-3-d)benzyl)-6,7-dihydro-5H-pyrrolo[3,4-b]pyridin-5-one-7,7-d₂;
6-(4-(pyrazolo[1,5-a]pyridin-7-yl)benzyl)-6,7-dihydro-5H-pyrrolo[3,4-b]pyridin-5-one-7,7-d₂;
6-(4-(2,4-dimethyl-2H-indazol-5-yl)-2-fluorobenzyl)-6,7-dihydro-5H-pyrrolo[3,4-b]pyridin-5-one-7,7-d₂;
6-(4-(1-(difluoromethyl)-1H-indazol-4-yl)-2,6-difluorobenzyl)-6,7-dihydro-5H-pyrrolo[3,4-b]pyridin-5-one-7,7-d₂;
6-(2,6-difluoro-4-(1-(methyl-d₃)-7-(trifluoromethyl)-1H-indazol-4-yl)benzyl)-6,7-dihydro-5H-pyrrolo[3,4-b]pyridin-5-one-7,7-d₂;
6-(4-(7-chloro-6-fluoro-2-methyl-2H-indazol-4-yl)-2,6-difluorobenzyl)-6,7-dihydro-5H-pyrrolo[3,4-b]pyridin-5-one-7,7-d₂;
6-(4-(6-chloro-1-(methyl-d₃)-1H-indazol-4-yl)-2,6-difluorobenzyl)-6,7-dihydro-5H-pyrrolo[3,4-b]pyridin-5-one-7,7-d₂;
6-(2,6-difluoro-4-(6-fluoro-1-(methyl-d₃)-1H-indazol-4-yl)benzyl)-6,7-dihydro-5H-pyrrolo[3,4-b]pyridin-5-one-7,7-d₂;
6-(4-(7-chloro-6-fluoro-2-(methyl-d₃)-2H-indazol-4-yl)-2,6-difluorobenzyl)-6,7-dihydro-5H-pyrrolo[3,4-b]pyridin-5-one-7,7-d₂;
6-(4-(7-chloro-6-fluoro-1-(methyl-d₃)-1H-indazol-4-yl)-2,6-difluorobenzyl)-6,7-dihydro-5H-pyrrolo[3,4-b]pyridin-5-one-7,7-d₂;
6-(2,6-difluoro-4-(7-isopropoxy-1-(methyl-d₃)-1H-indazol-4-yl)benzyl)-6,7-dihydro-5H-pyrrolo[3,4-b]pyridin-5-one-7,7-d₂;
6-(2,6-difluoro-4-(1-(methyl-d₃)-7-(trifluoromethoxy)-1H-indazol-4-yl)benzyl)-6,7-dihydro-5H-pyrrolo[3,4-b]pyridin-5-one-7,7-d₂;
6-(4-(6,7-difluoro-1-(methyl-d₃)-1H-indazol-4-yl)-2,6-difluorobenzyl)-6,7-dihydro-5H-pyrrolo[3,4-b]pyridin-5-one-7,7-d₂;
6-(2,6-difluoro-4-(7-methoxy-1-methyl-1H-pyrazolo[3,4-c]pyridin-4-yl)benzyl)-6,7-dihydro-5H-pyrrolo[3,4-b]pyridin-5-one-7,7-d₂;
6-(2,6-difluoro-4-(2-methylpyrazolo[1,5-a]pyridin-4-yl)benzyl)-6,7-dihydro-5H-pyrrolo[3,4-b]pyridin-5-one-7,7-d₂;
6-(2,6-difluoro-4-(2-(methyl-d₃)pyrazolo[1,5-a]pyridin-4-yl)benzyl)-6,7-dihydro-5H-pyrrolo[3,4-b]pyridin-5-one-7,7-d₂;
or a pharmaceutically acceptable salt thereof.

31. The compound of any of claims 1-30, or a pharmaceutically acceptable salt thereof, having a positive allosteric modulation of the mAChR M₁ response to acetylcholine with EC₅₀ of less than or equal to 0.5 μM.

32. A compound of any of claims 1-31, or a pharmaceutically acceptable salt thereof, that has at least 50% deuterium incorporation at each deuterium label.

33. A hydrate, solvate, polymorph, or prodrug of the compound of any of claims 1-32, or a pharmaceutically acceptable salt thereof.

34. A pharmaceutical composition comprising the compound, hydrate, solvate, polymorph, or prodrug of any of claims 1-33, or pharmaceutically acceptable salt thereof, and a pharmaceutically acceptable carrier.

35. A composition comprising:
(a) a compound of any of claims 1-32 according to formula (I), or a pharmaceutically acceptable salt, hydrate, solvate, polymorph, or prodrug thereof, and
(b) a compound according to formula (II), 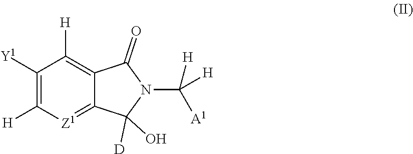
wherein Y¹, Z¹, and A¹ are as defined in any of claims 1-29 or 32, or a pharmaceutically acceptable salt, hydrate, solvate, polymorph, or prodrug thereof.

36. A method for the treatment of a disorder associated with muscarinic acetylcholine receptor activity in a mammal, comprising administering to the mammal an effective amount of the compound, hydrate, solvate, polymorph, or prodrug of any of claims 1-33, or a pharmaceutically acceptable salt thereof, or the composition of any of claims 34-35.

37. The method of claim 36, wherein the mammal is human.

38. The method of claim 36 or 37, wherein the muscarinic acetylcholine receptor is mAChR M₁.

39. The method of any of claims 36-38, wherein the mammal has been diagnosed with a need for treatment of the disorder prior to the administering step.

40. The method of any of claims 36-39, further comprising the step of identifying a mammal in need of treatment of the disorder.

41. The method of any of claims 36-40, wherein the disorder is a neurological disorder or psychiatric disorder, or a combination thereof.

42. The method of any of claims 36-41, wherein the disorder is psychosis, schizophrenia, conduct disorder, disruptive behavior disorder, bipolar disorder, psychotic episodes of anxiety, anxiety associated with psychosis, psychotic mood disorders, severe major depressive disorder, mood disorders associated with psychotic disorders, acute mania, depression associated with bipolar disorder, mood disorders associated with schizophrenia, behavioral manifestations of mental retardation, conduct disorder, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, or memory disorders, or a combination thereof.

43. The method of any of claims 36-41, wherein the disorder is Alzheimer's disease, schizophrenia, a sleep disorder, a pain disorder, or a cognitive disorder, or a combination thereof.

44. The method of claim 43, wherein the pain disorder is neuropathic pain, central pain syndrome, postsurgical pain syndrome, bone and joint pain, repetitive motion pain, dental pain, cancer pain, myofascial pain, perioperative pain, chronic pain, dysmennorhea, inflammatory pain, headache, migraine headache, cluster headache, headache, primary hyperalgesia, secondary hyperalgesis, primary allodynia, secondary allodynia, or a combination thereof.

45. The compound, hydrate, solvate, polymorph, or prodrug of any of claims 1-33, or a pharmaceutically acceptable salt thereof, or the composition of any of claims 34-35, for use in the treatment of a disorder associated with muscarinic acetylcholine receptor activity in a mammal.

46. Use of the compound, hydrate, solvate, polymorph, or prodrug of any of claims 1-33, or a pharmaceutically acceptable salt thereof, or the composition of any of claims 34-35, for the preparation of a medicament for the treatment of a disorder associated with muscarinic acetylcholine receptor activity in a mammal.
